(19) United States
(12) Patent Application Publication
(10) Pub. No.: US 2026/0285995 A1
(43) Pub. Date: Sep. 24, 2026

(54) CD70 CAR-T Compositions and Methods for Cell-based Therapy

(71) Applicant: Intellia Therapeutics, Inc., Cambridge, MA (US)

(72) Inventors: Michael Lam, Cambridge, MA (US); Ian Miller, Brookline, MA (US); Raghavender Siva Vijaya Chivukula, Wesley Chapel, FL (US); Aaron Prodeus, Cambridge, MA (US); Özgün Kiliç, Cambridge, MA (US); Palak Sharma, Watertown, MA (US); Dai Liu, Brookline, MA (US); Nachiket Narendra Shevale, North Billerica, MA (US); Birgit Schultes, Arlington, MA (US); Yong Zhang, Bedford, MA (US); Biao Liu, Cambridge, MA (US); Boning Zhang, Cambridge, MA (US); Ruan Oliveira, Boston, MA (US)

(73) Assignee: Intellia Therapeutics, Inc., Cambridge, MA (US)

(21) Appl. No.: 19/535,530

(22) Filed: Feb. 10, 2026

Related U.S. Application Data
(63) Continuation of application No. PCT/US24/42128, filed on Aug. 13, 2024.
(60) Provisional application No. 63/519,551, filed on Aug. 14, 2023.
(60) Provisional application No. 63/610,582, filed on Dec. 15, 2023.
(60) Provisional application No. 63/659,675, filed on Jun. 13, 2024.

(51) Int. Cl. C07K 16/28; A61K 35/17; A61K 40/11; A61K 40/15; A61K 40/31; A61K 40/42; A61K 40/50; A61P 35/02; C07K 14/705; C12N 5/0783; C12N 9/22; C12N 15/113; C12N 15/62; C12N 15/864; C12N 15/867; C12N 15/88

(52) U.S. Cl. CPC C07K 16/2875; A61K 35/17; A61K 40/11; A61K 40/15; A61K 40/31; A61K 40/4232; A61K 40/50; A61P 35/02; C07K 14/70517; C12N 5/0636; C12N 5/0646; C12N 9/226; C12N 15/113; C12N 15/62; C12N 15/8645; C12N 15/867; C12N 15/88; C07K 2317/53; C07K 2317/565; C07K 2317/622; C07K 2319/03; C12N 2310/20; C12N 2510/00; C12N 2740/15043; C12N 2750/14143

(57) ABSTRACT

Compositions and methods described herein relate to use of CRISPR/Cas9 systems in conjunction with CAR technology for improving the activity of an anti-CD70 CAR-T cell. The engineered cell may comprise a genetic modification in one or more of the HLA-A, HLA-B, TRAC, CIITA, TGFBR2, or CD70 gene.

### CROSS-REFERENCE TO RELATED APPLICATIONS

This application is a continuation application of International Application No. PCT/US2024/042128, filed Aug. 13, 2024, which claims the benefit under 35 USC 119 (e) of U.S. Provisional Application No. 63/519,551, filed Aug. 14, 2023, U.S. Provisional Application No. 63/610,582, filed Dec. 15, 2023, and U.S. Provisional Application No. 63/659,675, filed Jun. 13, 2024, the content of each of which is herein incorporated by reference in its entirety.

### REFERENCE TO ELECTRONIC SEQUENCE LISTING

This application contains a sequence listing, which has been submitted electronically in XML file format and is hereby incorporated by reference in its entirety. Said XML file, created on Aug. 12, 2024, is named “01155-0059-00PCT.xml” and is 3,148,975 bytes in size.

### INTRODUCTION AND SUMMARY

The present disclosure generally describes chimeric antigen receptor (CAR) reagents (e.g., cells and compositions) and methods. The present disclosure relates to use of CAR technology in conjunction with CRISPR systems for providing CAR-transduced immune effector cells (e.g., T-cells, NK cells).

CD70 is a transmembrane protein that is typically transiently expressed on the surface of CD4+ and CD8+ T-cells, regulatory T-cells (Tregs), B cells, antigen-presenting cells such as dendritic cells, and natural killer (NK) cells in response to immune activation.

Although CD70 expression is tightly controlled in normal tissue, many cancer cell types exhibit high levels of CD70. For example, CD70 is overexpressed in solid and liquid cancer types such as renal cell carcinoma (RCC), acute myeloid leukemia (AML), Hodgkin's and non-Hodgkin's lymphoma, multiple myeloma, pancreatic cancer, ovarian cancer, and non-small cell lung cancer (NSCLC). Notably, many of these cancer types have proved difficult-to-treat, creating a significant unmet need for new therapeutic approaches. On account of its differential expression on the surface of healthy and cancerous cells, CD70 represents an attractive target for emerging therapies, including immunotherapy.

Despite its therapeutic promise, anti-CD70 CAR-T therapy presents many logistical and technical hurdles. Current protocols for CAR-T are based on autologous cell transfer. In this approach, T-cells recovered from a patient are genetically modified ex vivo and cultivated in vitro before being infused into the patient. This approach, which uses the patient's own lymphocytes, reduces the risk of rejection. However, so-called autologous therapies are predicated on the availability of functional lymphocytes, which may be compromised by prior lines of treatment. Moreover, each patient's autologous cell preparation is effectively a new product, resulting in substantial variation in safety and efficacy.

“Off the shelf” therapies using donor (allogeneic) cells obviate the need to recover and modify the patient's own lymphocytes. However, allogeneic CAR-T cells may provoke an undesirable immune response or otherwise be short-lived. Often, immune rejection of allogeneic cells results from a mismatching of major histocompatibility complex (MHC) molecules between the donor and recipient. Slight differences, e.g., in MHC alleles between individuals can cause the T-cells in a recipient to become activated. During T-cell development, an individual's T-cell repertoire is tolerized to one's own MHC molecules, but T-cells that recognize another individual's MHC molecules may persist in circulation and are referred to as alloreactive T-cells. Alloreactive T-cells can become activated e.g., by the presence of another individual's cells expressing MHC molecules in the body, causing e.g., graft versus host disease and transplant rejection.

Methods and compositions for reducing the susceptibility of an allogeneic anti-CD70 CAR-T cell to rejection, or for improving the activity of an anti-CD70 CAR-T cell, are of interest.

Thus, there exists a need for improved methods and compositions for modifying anti-CD70 CAR-T cells to overcome the problem of recipient immune rejection, and to improve the activity of anti-CD70 CAR-T cells. The present disclosure provides genomic editing of anti-CD70 CAR-T cells by CRISPR/Cas9 systems.

The engineered cell comprises a genetic modification in the HLA-A, HLA-B, TRAC, CIITA (class II major histocompatibility complex transactivator), TGFBR2, or CD70 gene, which may be useful in cell therapy. The disclosure further provides compositions and methods to reduce or eliminate surface expression of endogenous T-cell receptor, MHC class I or II protein, CD70, or TGFBR2 in a cell by genetically modifying the HLA-A, HLA-B, TRAC, CIITA, TGFBR2, or CD70 locus.

In some embodiments, a method of reducing surface expression of HLA-A protein in an engineered cell relative to an unmodified cell, is provided, the method comprising contacting a cell with a composition of any of embodiments provided herein. In some embodiments, a method of reducing surface expression of HLA-B protein in an engineered cell relative to an unmodified cell, is provided, the method comprising contacting a cell with a composition of any of embodiments provided herein. In some embodiments, a method of reducing surface expression of TRAC protein in an engineered cell relative to an unmodified cell, is provided, the method comprising contacting a cell with a composition of any of embodiments provided herein. In some embodiments, a method of reducing surface expression of MHC class II protein in an engineered cell relative to an unmodified cell, is provided, the method comprising contacting a cell with a composition of any of embodiments provided herein. In some embodiments, a method of reducing surface expression of TGFBR2 protein in an engineered cell relative to an unmodified cell, is provided, the method comprising contacting a cell with a composition of any of embodiments provided herein. In some embodiments, a method of reducing surface expression of CD70 protein in an engineered cell relative to an unmodified cell, is provided, the method comprising contacting a cell with a composition of any of embodiments provided herein.

### BRIEF DESCRIPTION OF THE DRAWINGS

FIG. 1 shows a schematic of the anti-CD70 CAR construct with alternative antigen-binding proteins and co-stimulatory domains.

FIGS. 2A-2D show the in-vitro re-challenge of four anti-CD70 CAR constructs against a 786-O tumor cell line, measured by cancer cell area (mm2) and compared against a benchmark construct. FIG. 2A shows the re-challenge results for constructs 5719 and 5281, which share the same scFv antigen-binding protein domain, but which differ in certain intracellular domains. FIG. 2B shows the re-challenge results for constructs 5284 and 5718, which share the same scFv antigen-binding protein domain, but which differ in certain intracellular domains. FIG. 2C shows the re-challenge results for constructs 5283 and 5717, which share the same scFv antigen-binding protein domain, but which differ in certain intracellular domains. FIG. 2D shows the re-challenge results for construct 5715 with a 41BB costimulatory domain.

FIG. 3 compares the efficacy of five anti-CD70 CAR constructs in a 786-O mouse tumor cell model against a benchmark construct, measured by tumor volume (mm3). Constructs 5715, 5717, 5719, and 5718 each comprising a 41BB costimulatory domain and construct 5281 comprising a CD28 costimulatory domain were tested at a 3e6 dose.

FIG. 4 compares the efficacy of two anti-CD70 CAR constructs in a 786-O mouse tumor cell model against a benchmark construct, measured by tumor volume (mm3). Constructs 5719 and 5715 were tested at a 1e6 dose.

FIGS. 5A-5D show the impact of double (DKO) versus single (SKO) immune enhancing edit (IEE) knockouts with either construct 5719, construct 5718, or construct 4645 against a 786-O tumor cell line measured by the percent of viable tumor cells remaining. Unedited cells were used as a control. Constructs 5719 and 5718 were tested alone, with CD70 SKO, with TGFβR2 SKO, and with CD70+ TGFβR2 DKO. FIG. 5A shows the percent tumor cell viability for construct 5719 without the presence of TGFβ and FIG. 5B shows the results for construct 5719 in the presence of TGFβ. FIG. 5C shows the percent tumor cell viability for construct 5718 without the presence of TGFβ and FIG. 5D shows the results for construct 5718 in the presence of TGFβ.

FIGS. 6A-6D show the in-vitro re-challenge of four anti-CD70 CAR constructs alone, with SKO, or with DKO IEE edits against a 768-O tumor cell line, measured by tumor cell area (mm2). The constructs were compared to benchmark construct 4645 and TRAC KO alone. FIG. 6A shows the results for construct 5719, FIG. 6B shows the results for construct 5281, FIG. 6C shows the results for construct 5715, and FIG. 6D shows the results for construct 6115.

FIGS. 7A-7D show the in-vitro re-challenge of four anti-CD70 CAR constructs alone, with SKO, or with DKO IEE edits against an ACHN tumor cell line, measured by tumor cell area (mm2). The constructs were compared to benchmark construct 4645 and TRAC KO alone. FIG. 7A shows the results for construct 5719, FIG. 7B shows the results for construct 5281, FIG. 7C shows the results for construct 5715, and FIG. 7D shows the results for construct 6115.

FIGS. 8A-8C show the efficacy of three anti-CD70 CAR constructs alone, with SKO, or with DKO IEE edits in a 786-O mouse tumor cell model against benchmark construct 4645, measured by tumor volume (mm3). FIG. 8A shows the results for construct 5719, FIG. 8B shows the results for construct 5715, and FIG. 8C shows the results for construct 5281.

FIG. 9A-9G show the re-challenge results measured by tumor volume (mm3) for the anti-CD70 CAR constructs with either SKO or DKO IEE edits that fully controlled tumor growth in FIGS. 8A-C. Constructs were compared to mice with tumor only. FIG. 9A shows the re-challenge results for construct 5719+CD70 KO. FIG. 9B shows the re-challenge results for construct 5719+ TGFβR2 KO. FIG. 9C shows the re-challenge results for construct 5715+CD70+ TGFβR2 DKO. FIG. 9D shows the re-challenge results for construct 5281+ TGFβR2 KO. FIG. 9E shows the re-challenge results for construct 5281+CD70+ TGFβR2 DKO. FIG. 9F shows the re-challenge results for construct 5719+CD70+TGFβR2 DKO. FIG. 9G shows the re-challenge results for construct 5715+ TGFβR2 KO.

FIG. 10 shows percentage of editing for each edit of the allogeneic edited CD70 CAR-T cells across three donors as assessed by flow cytometry or by genomic sequencing (results from each donor are shown in solid dots).

FIGS. 11A-11C show the percent of CAR T cells that present specified activation markers. FIG. 11A shows the percent of CAR T cells positive for CD69, FIG. 11B shows the percent of CAR T cells positive for CD107a, and FIG. 11C shows the percent of CAR T cells positive for CD25.

FIGS. 12A-12B show the re-challenge results measured by number of tumor cells with three different lots of CAR-T cells against a high CD70 and a medium CD70 expressing tumor cell line. FIG. 12A shows the re-challenge results for T cells challenged against the 786-O tumor cell line and FIG. 12B shows the re-challenge results for T cells challenged against the ACHN tumor cell line.

FIG. 13 shows the efficacy as measured by tumor volume (mm3) of two different lots of T cells at three different doses (10e6, 3e6, 1e6) against 786-O tumor cells over the course of 115 days.

FIG. 14 shows the efficacy as measured by tumor volume (mm3) of engineered T cells against 11 different PDX tumor models over the course of 42 days.

FIG. 15 shows karyotyping data comparing edited cells with donor matched unedited controls. 200 cell spreads were analyzed for each sample (N=3 donors). Statistical analysis was performed for each indicated aberration on a donor wise basis using Fisher's Exact Test. * denotes p<0.05 for any donor set. Bars represent mean±/−SD from three matched donors (dots).

FIGS. 16A-16B show the average percent of engineered donor T cell killing (either all donors of B2M CD70-CAR T cell group or all donors of Allo CD70-CAR T cell group) by host NK cells as normalized to a CAR alone group following treatment with genotypically mismatched or HLA-C-matched host NK cells. FIG. 16A shows the results for a genotypically mismatched system and FIG. 16B shows the results for a HLA-C-matched system.

FIGS. 17A-17C show the average percent proliferation of engineered donor T cells (either all donors of CAR alone group (solid circles) or all donors of allo CD70 CAR T cell group (solid squares)) as compared to a normalized value following treatment with genotypically mismatched or C-matched host PBMC. FIG. 17A shows the results for a genotypically mismatched system and FIG. 17B shows the results for a C-matched system.

FIG. 17C shows the average percent proliferation of engineered donor T cells in the presence of autologous PBMCs.

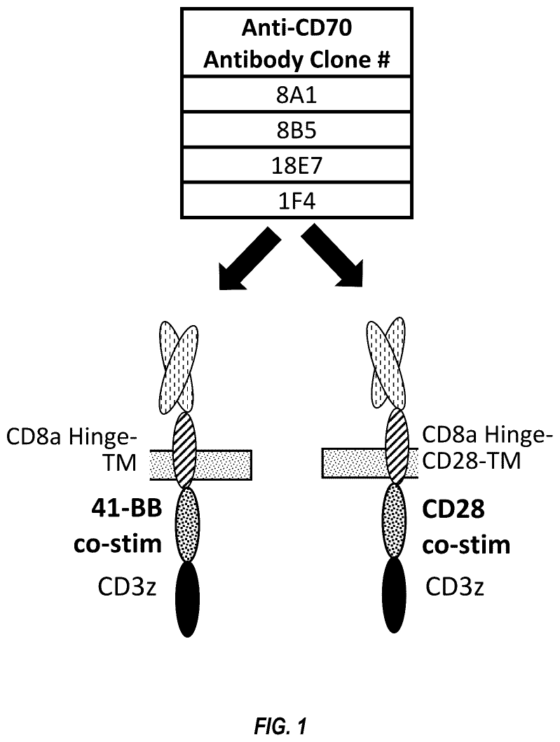

### DETAILED DESCRIPTION

The present disclosure provides for chimeric antigen receptors (CARs), as well as methods and compositions for making immune effector cells (e.g., T-cells and NK cells) comprising CARs. In embodiments the cell is a T-cell which is engineered to express a CAR, e.g., as described herein. In embodiments, the CAR is an anti-CD70 CAR, e.g., as described herein.

In some embodiments, the disclosure provided herein further relates to the use of CAR technology in conjunction with genome editing using a CRISPR/Cas system, e.g., a Cas9 system. In some embodiments, the present disclosure describes genetically modified CAR-transduced cells, as well gRNA molecules, compositions, and methods for genetically modifying CAR-transduced cells. In particular, the gRNA molecules, compositions and methods described herein relate to regulation of expression of (or expression of functional versions of) target molecules that have an effect on the function of a transplanted cell, for example a cell for cancer immunotherapy. In one embodiment, the transplanted cell is an immune effector cell, e.g., an NK cell or T-cell. In one embodiment, the cell is an allogeneic cell. Thus, provided herein are compositions and methods for altering, e.g., inhibiting or reducing, the expression and/or function (e.g., the level of expression of a functional version) of a gene target or a protein encoded by a gene target, thereby improving the efficacy (for example, by reducing or eliminating undesirable immunogenicity (such as a host versus graft response or a graft versus host response)), function, proliferation, stimulation or survival of a transplanted cell, for example a transplanted immune effector cell, e.g., a NK cell or T-cell, e.g., a T-cell engineered to express a CAR, e.g., an allogeneic CAR-expressing T-cell for immunotherapy.

In some embodiments, the gene targets are allogeneic T-cell proteins such as an HLA-A, HLA-B, TRAC, or CIITA. Without being bound by theory it is believed that inhibition or elimination of the level of an allogeneic T-cell target or level of expression of an allogeneic T-cell target gene target (e.g., via alteration of the gene) may improve the function of a cell, e.g., a transplanted cell, e.g., a transplanted immune effector cell, e.g., a CAR-T cell, e.g., an allogeneic CAR-T cell, by reducing or eliminating a graft vs. host response, a host vs. graft response, or will render said transplanted cell resistant to immunosuppressant therapy.

In an aspect, compositions and methods described herein can be used to improve cell, e.g., T-cell, e.g., CAR-engineered T-cell, e.g., allogeneic CAR-engineered T-cell function (for example, by reducing or eliminating undesirable immunogenicity (such as a host versus graft response or a graft versus host response)), survival, proliferation and/or efficacy by altering the gene of a component of the major histocompatibility complex, e.g., an HLA protein, e.g., HLA-A and/or HLA-B. While not wishing to be bound by theory, it is considered that reduced or absent expression of a mismatch (e.g., one that does not match the type of the subject receiving the cell therapy) HLA protein (or component) reduces or eliminates host vs. graft disease by eliminating host T-cell receptor recognition of and response to mismatched (e.g., allogeneic) graft tissue. This approach, therefore, could be used to generate “off the shelf” T-cells (Torikai et al., 2012 Blood 119, 5697-5705).

In an aspect, compositions and methods described herein can be used to improve cell, e.g., T-cell, e.g., CAR-engineered T-cell, e.g., allogeneic CAR-engineered T-cell function (for example, by reducing or eliminating undesirable immunogenicity (such as a host versus graft response or a graft versus host response)), survival, proliferation and/or efficacy by altering the gene of a component of the T-cell receptor (TCR), e.g., TRAC. While not wishing to be bound by theory, it is considered that reduced or absent expression of functional T-cell receptor components reduces or eliminates the presence of TCR on the surface of said cell, thereby reducing or preventing graft vs. host disease by eliminating T-cell receptor recognition of and response to host tissues. This approach, therefore, could be used to generate “off the shelf” T-cells.

In an aspect, compositions and methods described herein can be used to improve cell, e.g., T-cell, e.g., CAR-engineered T-cell, e.g., allogeneic CAR-engineered T-cell function (for example, by reducing or eliminating undesirable immunogenicity (such as a host versus graft response or a graft versus host response)), survival, proliferation and/or efficacy by altering the gene encoding a protein that regulates expression of one or more components of the major histocompatibility complex, e.g., CIITA. While not wishing to be bound by theory, it is believed that reducing or eliminating the expression of a regulator of MHC class II expression, e.g., CIITA, will reduce or eliminate the expression of MHC class II molecules on the allogeneic cell, thereby reducing or eliminating expression of a mismatch (e.g., one that does not match the type of the subject receiving the cell therapy) MHC class II protein (or component), thereby reducing or eliminating host vs. graft disease by, e.g., eliminating host T-cell receptor recognition of and response to mismatched (e.g., allogeneic) graft tissue, e.g., allogeneic T-cell, e.g., allogeneic CAR-T cell, as described herein. This approach, therefore, could be used to generate “off the shelf” T-cells.

In one embodiment, it may be beneficial to reduce or eliminate expression of one or more MHC class I molecules and one or more MHC II molecules, e.g., in a T-cell, e.g., in an allogeneic T-cell, e.g., in an allogeneic CAR-T cell, e.g., as described herein, to further reduce or eliminate the host versus graft disease response upon administration of the cell. Thus, in embodiments of the cells and methods of the present disclosure, cells may be contacted with a composition of the present disclosure (e.g., a composition comprising a gRNA and a Cas9 molecule) comprising a gRNA molecule, e.g., as described herein, to HLA-A or HLA-B (e.g., such that expression of one or more MHC class I molecules is reduced or eliminated in said cell) and a composition of the present disclosure (e.g., a composition comprising a gRNA and a Cas9 molecule) comprising a gRNA molecule, e.g., as described herein, to CIITA (e.g., such that expression of one or more MHC class II molecules is reduced or eliminated). In embodiments of the cells and methods of the present disclosure, the cell may also be contacted with a composition of the present disclosure (e.g., a composition comprising a gRNA and a Cas9 molecule) comprising a gRNA molecule, e.g., as described herein, to a component of the TCR, e.g., to TRAC (e.g., such that expression of the T-cell receptor, e.g., one or more components of the TCR is reduced or eliminated). In an embodiment, a cell of the present disclosure has reduced or eliminated expression of TCR (e.g., as detected by flow cytometry), reduced or eliminated expression of one or more MHC class I molecules (e.g., as detected by flow cytometry), and reduced or eliminated expression of one or more MHC class II molecules (e.g., as detected by flow cytometry).

In embodiments, the reduced or eliminated expression is measured relative to a similar cell that has not been treated with a composition or CRISPR system of the present disclosure. In embodiments, the cell is an immune effector cell, e.g., a T-cell or NK cell, e.g., a T-cell, e.g., as described herein. In embodiments, the cell is a human cell. In embodiments, the cell is allogeneic relative to a subject to be administered said cell. In embodiments, the reduced or eliminated expression of HLA-A, HLA-B, TRAC, and/or CIITA is accomplished by introducing into said cell a composition, CRISPR system, or gRNA of the present disclosure, e.g., as described herein, or by a method as described herein.

In another embodiment, compositions and methods described herein can be used to improve cell, e.g., T-cell, e.g., CAR-transduced T-cell, e.g., allogeneic CAR-transduced T-cell function (for example, by reducing or eliminating undesirable immunogenicity (such as hast versus graft response or a graft versus host response)), survival, proliferation, and/or efficacy by altering the gene of an immune signaling protein, e.g., TGFβR2 or CD70. This approach, therefore, could be used to generate “off the shelf” T-cells.

In embodiments, the reduced or eliminated expression is measured relative to a similar cell that has not been treated with a composition or CRISPR system of the present disclosure. In embodiments, the cell is an immune effector cell, e.g., a T-cell or NK cell, e.g., a T-cell, e.g., as described herein. In embodiments, the cell is a human cell. In embodiments, the cell is allogeneic relative to a subject to be administered said cell. In embodiments, the reduced or eliminated expression of TGFβR2 or CD70 is accomplished by introducing into said cell a composition, CRISPR system, or gRNA of the present disclosure, e.g., as described herein, or by a method as described herein.

In embodiments in which the cells have reduced or eliminated levels or expression levels of HLA-A, the cells comprises a genetic modification within genomic coordinates targeted by a guide RNA comprising a guide sequence of any one of SEQ ID NOs: 403, 404, and 412, or a gRNA molecule comprising a guide sequence consisting of 20, 21, 22, 23, 24 or 25 consecutive nucleotides, preferably 20 consecutive nucleotides, of any one of SEQ ID NOs: 403, 404, and 412.

In embodiments in which the cells have reduced or eliminated levels or expression levels of HLA-B, the cells comprises a genetic modification within genomic coordinates targeted by a guide RNA comprising a guide sequence of any one of SEQ ID NOs: 405-407, or a gRNA molecule comprising a guide sequence consisting of 17, 18, 19, 20, 21, 22, 23, 24 or 25 consecutive nucleotides, preferably 20 consecutive nucleotides, of any one of SEQ ID NOs: 405-407.

In embodiments in which the cells have reduced or eliminated levels or expression levels of TRAC, the cells comprise a genetic modification within genomic coordinates targeted by a guide RNA comprising a guide sequence of SEQ ID NO: 413, or a gRNA molecule comprising a guide sequence consisting of 17, 18, 19, 20, 21, 22, 23, 24 or 25 consecutive nucleotides, preferably 20 consecutive nucleotides, of SEQ ID NO: 413.

In embodiments in which the cells have reduced or eliminated levels or expression levels of CIITA, the cells comprises a genetic modification within genomic coordinates targeted by a guide RNA comprising a guide sequence of any one of SEQ ID NOs: 401, 402, and 411, or a gRNA molecule comprising a guide sequence consisting of 17, 18, 19, 20, 21, 22, 23, 24 or 25 consecutive nucleotides, preferably 20 consecutive nucleotides, of any one of SEQ ID NOs: 401, 402, and 411.

In embodiments in which the cells have reduced or eliminated levels or expression levels of TGFβR2, the cells comprises a genetic modification within genomic coordinates targeted by a guide RNA comprising a guide sequence of any one of SEQ ID NOs: 301-309, 371, or 372, or a gRNA molecule comprising a guide sequence consisting of 17, 18, 19, 20, 21, 22, 23, 24 or 25 consecutive nucleotides, preferably 20 consecutive nucleotides, of any one of SEQ ID NOs: 301-309, 371, or 372.

In embodiments in which the cells have reduced or eliminated levels or expression levels of CD70, the cells comprises a genetic modification within genomic coordinates targeted by a guide RNA comprising a guide sequence of any one of SEQ ID NOs: 310-319, or a gRNA molecule comprising a guide sequence consisting of 17, 18, 19, 20, 21, 22, 23, 24 or 25 consecutive nucleotides, preferably 20 consecutive nucleotides, of any one of SEQ ID NOs: 310-319.

The term “about” or “approximately” means an acceptable error for a particular value as determined by one of ordinary skill in the art, which depends in part on how the value is measured or determined, or a degree of variation that does not substantially affect the properties of the described subject matter, or within the tolerances accepted in the art, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The following numbered embodiments are provided. Additional embodiments are provided throughout the present disclosure.

Embodiment 1 is an anti-CD70 chimeric antigen receptor (CAR) comprising: a. an antigen-binding protein, or a fragment thereof, that specifically binds to CD70, wherein the antigen-binding protein comprises a heavy chain variable (VH) region comprising a complementarity determining region 1 (VH CDR1), VH CDR2, and VH CDR3, and a light chain variable (VL) region comprising a complementarity determining region 1 (VL CDR1), VL CDR2, and VL CDR3, and wherein i. the VH CDR1 comprises an amino acid sequence of any one of SEQ ID NOs: 67, 70, 73, 76, 79, and 82; ii. the VH CDR2 comprises an amino acid sequence of any one of SEQ ID NOs: 68, 71, 74, 77, 80, and 83; iii, the VH CDR3 comprises an amino acid sequence of any one of SEQ ID NOs: 69, 72, 75, 78, 81, and 84; iv. the VL CDR1 comprises an amino acid sequence of any one of SEQ ID NOs: 49, 52, 55, 58, 61, and 64; v. the VL CDR2 comprises an amino acid sequence of any one of SEQ ID NOs: 50, 53, 56, 59, 62, and 65; and vi. the VL CDR3 comprises an amino acid sequence of any one of SEQ ID NOs: 51, 54, 57, 60, 63, and 66; b. a transmembrane domain; and c. an intracellular domain comprising a costimulatory domain comprising an amino acid sequence of SEQ ID NO: 99 or 101.

Embodiment 1.1 is the anti-CD70 CAR of embodiment 1, wherein the VH CDR1, VH CDR2, VH CDR3, VL CDR1, VL CDR2, and VL CDR3 comprise the amino acid sequences of: (a) SEQ ID NOs: 73, 74, 75, 55, 56, and 57, respectively; (b) SEQ ID NOs: 67, 68, 69, 49, 50, and 51, respectively; (c) SEQ ID NOs: 70, 71, 72, 52, 53, and 54, respectively; (d) SEQ ID NOs: 76, 77, 78, 58, 59, and 60, respectively; (e) SEQ ID NOs: 79, 80, 81, 61, 62, and 63, respectively; or (f) SEQ ID NOs: 82, 83, 84, 64, 65, and 66, respectively.

Embodiment 1.2 is the anti-CD70 CAR of embodiment 1 or 2, wherein the VH CDR1, VH CDR2, VH CDR3, VL CDR1, VL CDR2, and VL CDR3 comprise the amino acid sequences of: SEQ ID NOs: 73, 74, 75, 55, 56, and 57, respectively.

Embodiment 2 is the anti-CD70 CAR of any one of embodiments 1, 1.1, and 1.2, wherein the VH region comprises an amino acid sequence having at least 90%, 93%, 95%, 96%, 97%, 98%, or 99% identity to any one of SEQ ID NOs: 43-48.

Embodiment 3 is the anti-CD70 CAR of any one of embodiments 1, 1.1, 1.2, and 2, wherein the VL region comprises an amino acid sequence having at least 90%, 93%, 95%, 96%, 97%, 98%, or 99% identity to any one of SEQ ID NOs: 37-42.

Embodiment 3.1 is the anti-CD70 CAR of any one of embodiments 1, 1.1, 1.2, 2, and 3, wherein: (a) the VH region comprises an amino acid sequence having at least 90%, 93%, 95%, 96%, 97%, 98%, or 99% identity to SEQ ID NO: 45, and the VL region comprises an amino acid sequence having at least 90%, 93%, 95%, 96%, 97%, 98%, or 99% identity to SEQ ID NO: 39; (b) the VH region comprises an amino acid sequence having at least 90%, 93%, 95%, 96%, 97%, 98%, or 99% identity to SEQ ID NO: 43, and the VL region comprises an amino acid sequence having at least 90%, 93%, 95%, 96%, 97%, 98%, or 99% identity to SEQ ID NO: 37; (c) the VH region comprises an amino acid sequence having at least 90%, 93%, 95%, 96%, 97%, 98%, or 99% identity to SEQ ID NO: 44, and the VL region comprises an amino acid sequence having at least 90%, 93%, 95%, 96%, 97%, 98%, or 99% identity to SEQ ID NO: 38; (d) the VH region comprises an amino acid sequence having at least 90%, 93%, 95%, 96%, 97%, 98%, or 99% identity to SEQ ID NO: 46, and the VL region comprises an amino acid sequence having at least 90%, 93%, 95%, 96%, 97%, 98%, or 99% identity to SEQ ID NO: 40; (e) the VH region comprises an amino acid sequence having at least 90%, 93%, 95%, 96%, 97%, 98%, or 99% identity to SEQ ID NO: 47, and the VL region comprises an amino acid sequence having at least 90%, 93%, 95%, 96%, 97%, 98%, or 99% identity to SEQ ID NO: 41; or (f) the VH region comprises an amino acid sequence having at least 90%, 93%, 95%, 96%, 97%, 98%, or 99% identity to SEQ ID NO: 48, and the VL region comprises an amino acid sequence having at least 90%, 93%, 95%, 96%, 97%, 98%, or 99% identity to SEQ ID NO: 42.

Embodiment 3.2 is the anti-CD70 CAR of any one of embodiments 1, 1.1, 1.2, 2, 3, and 3.1, wherein: the VH region comprises an amino acid sequence having at least 90%, 93%, 95%, 96%, 97%, 98%, or 99% identity to SEQ ID NO: 45, and the VL region comprises an amino acid sequence having at least 90%, 93%, 95%, 96%, 97%, 98%, or 99% identity to SEQ ID NO: 39.

Embodiment 3.3 is the anti-CD70 CAR of any one of embodiments 1, 1.1, 1.2, 2, 3, 3.1, and 3.2, wherein: (a) the VH region comprises the amino acid sequence of SEQ ID NO: 45, and the VL region comprises the amino acid sequence of SEQ ID NO: 39; (b) the VH region comprises the amino acid sequence of SEQ ID NO: 43, and the VL region comprises the amino acid sequence of SEQ ID NO: 37; (c) the VH region comprises the amino acid sequence of SEQ ID NO: 44, and the VL region comprises the amino acid sequence of SEQ ID NO: 38; (d) the VH region comprises the amino acid sequence of SEQ ID NO: 46, and the VL region comprises the amino acid sequence of SEQ ID NO: 40; (e) the VH region comprises the amino acid sequence of SEQ ID NO: 47, and the VL region comprises the amino acid sequence of SEQ ID NO: 41; or (f) the VH region comprises the amino acid sequence of SEQ ID NO: 48, and the VL region comprises the amino acid sequence of SEQ ID NO: 42.

Embodiment 3.4 is the anti-CD70 CAR of any one of embodiments 1, 1.1, 1.2, 2, 3, and 3.1-3.3, wherein: the VH region comprises the amino acid sequence of SEQ ID NO: 45, and the VL region comprises the amino acid sequence of SEQ ID NO: 39.

Embodiment 4 is the anti-CD70 CAR of any one of embodiments 1-3, 1.1, 1.2, and 3.1-3.4, wherein the antigen-binding protein comprises an amino acid sequence of any one of SEQ ID NOs: 25, 27, 29, 32, 34, and 36, or a sequence having at least 90%, 93%, 95%, 96%, 97%, 98%, or 99% identity to any one of SEQ ID NOs: 25, 27, 29, 32, 34, and 36.

Embodiment 5 is the anti-CD70 CAR of any one of embodiments 1-4, 1.1, 1.2, and 3.1-3.4, wherein the antigen-binding protein comprises the amino acid sequence of SEQ ID NO: 25.

Embodiment 6 is the anti-CD70 CAR of any one of embodiments 1-4, 1.1, 1.2, and 3.1-3.4, wherein the antigen-binding protein comprises the amino acid sequence of SEQ ID NO: 27.

Embodiment 7 is the anti-CD70 CAR of any one of embodiments 1-4, 1.1, 1.2, and 3.1-3.4, wherein the antigen-binding protein comprises an amino acid sequence of SEQ ID NO: 25 or SEQ ID NO: 27 and the costimulatory domain comprises the amino acid sequence of SEQ ID NO: 99.

Embodiment 8 is the anti-CD70 CAR of any one of embodiments 1-4, 1.1, 1.2, and 3.1-3.4, wherein the antigen-binding protein comprises an amino acid sequence of SEQ ID NO: 25 or SEQ ID NO: 27 and the costimulatory domain comprises the amino acid sequence of SEQ ID NO: 101.

Embodiment 9 is the anti-CD70 CAR of any one of embodiments 1-8, 1.1, 1.2, and 3.1-3.4, wherein the transmembrane domain comprises a CD8a transmembrane domain comprising an amino acid sequence of SEQ ID NO: 95.

Embodiment 10 is the anti-CD70 CAR of any one of embodiments 1-8, 1.1, 1.2, and 3.1-3.4, wherein the transmembrane domain comprises a CD28 transmembrane domain comprising an amino acid sequence of SEQ ID NO: 93.

Embodiment 11 is the anti-CD70 CAR of any one of embodiments 1-10, 1.1, 1.2, and 3.1-3.4, further comprising a hinge domain between the antigen-binding protein and the transmembrane domain.

Embodiment 12 is the anti-CD70 CAR of embodiment 11, wherein the hinge domain is a CD8a hinge domain, or fragment thereof, comprising an amino acid sequence of SEQ ID NO: 89.

Embodiment 13 is the anti-CD70 CAR of any one of embodiments 1-12, 1.1, 1.2, and 3.1-3.4, wherein the intracellular domain further comprises an activation domain.

Embodiment 14 is the anti-CD70 CAR of embodiment 13, wherein the activation domain is a CD3z activation domain comprising an amino acid sequence of SEQ ID NO: 103.

Embodiment 15 is the anti-CD70 CAR of any one of embodiments 1-14, 1.1, 1.2, and 3.1-3.4, wherein the intracellular domain comprises a CD28 costimulatory domain comprising the amino acid sequence of SEQ ID NO: 99 and a CD3z activation domain comprising the amino acid sequence of SEQ ID NO: 103.

Embodiment 16 is the anti-CD70 CAR of any one of embodiments 1-15, 1.1, 1.2, and 3.1-3.4, wherein the intracellular domain comprises a 41BB costimulatory domain comprising the amino acid sequence of SEQ ID NO: 101 and a CD3z activation domain comprising the amino acid sequence of SEQ ID NO: 103.

Embodiment 17 is the anti-CD70 CAR of any one of embodiments 1-16, wherein: a. the antigen-binding protein comprises a sequence of SEQ ID NO: 32 and the costimulatory domain comprises a sequence of SEQ ID NO: 101; b. the antigen-binding protein comprises a sequence of SEQ ID NO: 32 and the costimulatory domain comprises a sequence of SEQ ID NO: 99; c. the antigen-binding protein comprises a sequence of SEQ ID NO: 25 and the costimulatory domain comprises a sequence of SEQ ID NO: 101; d. the antigen-binding protein comprises a sequence of SEQ ID NO: 25 and the costimulatory domain comprises a sequence of SEQ ID NO: 99; e. the antigen-binding protein comprises a sequence of SEQ ID NO: 27 and the costimulatory domain comprises a sequence of SEQ ID NO: 101; f. the antigen-binding protein comprises a sequence of SEQ ID NO: 27 and the costimulatory domain comprises a sequence of SEQ ID NO: 99; g. the antigen-binding protein comprises a sequence of SEQ ID NO: 36 and the costimulatory domain comprises a sequence of SEQ ID NO: 101; or h. the antigen-binding protein comprises a sequence of SEQ ID NO: 36 and the costimulatory domain comprises a sequence of SEQ ID NO: 99.

Embodiment 18 is the anti-CD70 CAR of any one of embodiments 1-17, 1.1, 1.2, and 3.1-3.4, comprising an amino acid sequence that is at least 95% identical to the amino acid sequence of any one of SEQ ID NOs: 2, 4, 6, 8, 14, 16, 20, and 22.

Embodiment 19 is the anti-CD70 CAR of any one of embodiments 1-18, 1.1, 1.2, and 3.1-3.4, comprising an amino acid sequence that is at least 95% identical to the amino acid sequence of SEQ ID NO: 4.

Embodiment 20 is the anti-CD70 CAR of any one of embodiments 1-19, 1.1, 1.2, and 3.1-3.4, comprising an amino acid sequence that is at least 95% identical to the amino acid sequence of SEQ ID NO: 8.

Embodiment 21 is the anti-CD70 CAR of any one of embodiments 1-20, 1.1, 1.2, and 3.1-3.4, comprising an amino acid sequence that is at least 95% identical to the amino acid sequence of SEQ ID NO: 2.

Embodiment 22 is the anti-CD70 CAR of any one of embodiments 1-21, 1.1, 1.2, and 3.1-3.4, wherein the antigen-binding protein is an scFv.

Embodiment 23 is the anti-CD70 CAR of embodiment 22, wherein the scFv comprises a linker between the VH and VL regions.

Embodiment 24 is the anti-CD70 CAR of embodiment 22 or 23, wherein the scFv comprises a glycine-serine linker between the VH and VL regions, optionally the glycine-serine linker comprising the sequence of SEQ ID NO: 940.

Embodiment 25 is a nucleic acid encoding the anti-CD70 CAR of any one of embodiments 1-24, 1.1, 1.2, and 3.1-3.4.

Embodiment 26 is the nucleic acid of embodiment 25, comprising a nucleic acid sequence of any one of SEQ ID NOs: 23, 24, 26, 28, 30, 31, 33, and 35, or a nucleic acid sequence that has at least 90%, 93%, 95%, 96%, 97%, 98%, or 99% identity to any one of SEQ ID NOs: 23, 24, 26, 28, 30, 31, 33, and 35.

Embodiment 27 is the nucleic acid of embodiment 25 or 26, comprising a nucleic acid sequence of any one of SEQ ID NOs: 96-98 and 100, or a nucleic acid sequence that has at least 90%, 93%, 95%, 96%, 97%, 98%, or 99% identity to any one of SEQ ID NOs: 96-98 and 100.

Embodiment 28 is the nucleic acid of any one of embodiments 25-27, wherein the anti-CD70 CAR comprises a hinge domain, wherein the nucleic acid comprises a nucleic acid encoding the hinge domain comprising a nucleic acid sequence of any one of SEQ ID NOs: 85-88 or a nucleic acid sequence that has at least 90%, 93%, 95%, 96%, 97%, 98%, or 99% identity to any one of SEQ ID NOs: 85-88.

Embodiment 29 is the nucleic acid of any one of embodiments 25-28, comprising a nucleic acid sequence encoding the transmembrane domain comprising a sequence of any one of SEQ ID NOs: 90-92 and 94 or a sequence having at least 90%, 93%, 95%, 96%, 97%, 98%, or 99% identity to any one of SEQ ID NOs: 90-92 and 94.

Embodiment 30 is the nucleic acid of any one of embodiments 25-29, wherein the intracellular domain comprises an activation domain, wherein the nucleic acid comprises a nucleic acid encoding the activation domain that comprises a nucleic acid sequence of SEQ ID NO: 102 or 104 or a nucleic acid sequence that has at least 90%, 93%, 95%, 96%, 97%, 98%, or 99% identity to SEQ ID NO: 102 or 104.

Embodiment 31 is the nucleic acid of any one of embodiments 25-30, comprising a nucleic acid sequence of any one of SEQ ID NOs: 1, 3, 5, 7, 9, 11, 13, 15, 17, 19, and 21, or a nucleic acid sequence that has at least 90%, 93%, 95%, 96%, 97%, 98%, or 99% identity to any one of SEQ ID NOs: 1, 3, 5, 7, 9, 11, 13, 15, 17, 19, and 21.

Embodiment 32 is the nucleic acid of any one of embodiments 25-31, comprising a nucleic acid sequence of any one of SEQ ID NOs: 1, 3, and 7.

Embodiment 33 is an mRNA encoded by the nucleic acid of any one of embodiments 25-32.

Embodiment 34 is an expression vector operably linked to or comprising the nucleic acid of any one of embodiments 25-32.

Embodiment 35 is an engineered cell comprising the nucleic acid of any one of embodiments 25-32, the mRNA of embodiment 33, or the expression vector of embodiment 34.

Embodiment 36 is an engineered cell comprising the anti-CD70 CAR of any one of embodiments 1-24, 1.1, 1.2, and 3.1-3.4.

Embodiment 37 is an engineered cell comprising the anti-CD70 CAR of any one of embodiments 1-24, 1.1, 1.2, and 3.1-3.4, wherein the cell is transduced with an expression vector operably linked to or comprising a nucleic acid encoding the anti-CD70 CAR, and wherein the expression vector directs expression of the anti-CD70 CAR in the cell.

Embodiment 38 is the expression vector of embodiment 34, or the engineered cell of embodiment 35 or 37, wherein the expression vector comprises a retroviral or lentiviral expression vector.

Embodiment 39 is the expression vector of embodiment 34, or the engineered cell of embodiment 35 or 37, wherein the expression vector comprises an AAV vector.

Embodiment 40 is the expression vector or engineered cell of embodiment 39, wherein the expression vector comprises SEQ ID NO: 106.

Embodiment 41 is the engineered cell of embodiment 40, wherein the engineered cell comprises SEQ ID NO: 107.

Embodiment 42 is an engineered cell comprising an anti-CD70 chimeric antigen receptor (CAR), wherein the anti-CD70 CAR comprises: a. an antigen-binding protein, or a fragment thereof, that specifically binds to CD70, wherein the antigen-binding protein comprises a heavy chain variable (VH) region comprising a complementarity determining region 1 (VH CDR1), VH CDR2, and VH CDR3, and a light chain variable (VL) region comprising a complementarity determining region 1 (VL CDR1), VL CDR2, and VL CDR3, and wherein i. the VH CDR1 comprises an amino acid sequence of any one of SEQ ID NOs: 67, 70, 73, 76, 79, and 82; ii. the VH CDR2 comprises an amino acid sequence of any one of SEQ ID NOs: 68, 71, 74, 77, 80, and 83; iii, the VH CDR3 comprises an amino acid sequence of any one of SEQ ID NOs: 69, 72, 75, 78, 81, and 84; iv. the VL CDR1 comprises an amino acid sequence of any one of SEQ ID NOs: 49, 52, 55, 58, 61, and 64; v. the VL CDR2 comprises an amino acid sequence of any one of SEQ ID NOs: 50, 53, 56, 59, 62, and 65; vi. the VL CDR3 comprises an amino acid sequence of any one of SEQ ID NOs: 51, 54, 57, 60, 63, and 66; b. a transmembrane domain; and c. an intracellular domain comprising a costimulatory domain comprising an amino acid sequence of SEQ ID NO: 99 or 101.

Embodiment 42.1 is the engineered cell of embodiment 42, wherein the VH CDR1, VH CDR2, VH CDR3, VL CDR1, VL CDR2, and VL CDR3 comprise the amino acid sequences of: (a) SEQ ID NOs: 73, 74, 75, 55, 56, and 57, respectively; (b) SEQ ID NOs: 67, 68, 69, 49, 50, and 51, respectively; (c) SEQ ID NOs: 70, 71, 72, 52, 53, and 54, respectively; (d) SEQ ID NOs: 76, 77, 78, 58, 59, and 60, respectively; (e) SEQ ID NOs: 79, 80, 81, 61, 62, and 63, respectively; or (f) SEQ ID NOs: 82, 83, 84, 64, 65, and 66, respectively.

Embodiment 42.2 is the engineered cell of embodiment 42 or 42.1, wherein the VH CDR1, VH CDR2, VH CDR3, VL CDR1, VL CDR2, and VL CDR3 comprise the amino acid sequences of: SEQ ID NOs: 73, 74, 75, 55, 56, and 57, respectively.

Embodiment 43 is the engineered cell of any one of embodiments 42, 42.1, and 42.2, wherein the VH region comprises an amino acid sequence having at least 90%, 93%, 95%, 96%, 97%, 98%, or 99% identity to any one of SEQ ID NOs: 43-48.

Embodiment 44 is the engineered cell of any one of embodiments 42, 42.1, 42.2, and 43, wherein the VL region comprises an amino acid sequence having at least 90%, 93%, 95%, 96%, 97%, 98%, or 99% identity to any one of SEQ ID NOs: 37-42.

Embodiment 44.1 is the engineered cell of any one of embodiments 42, 42.1, 42.2, 43, and 44, wherein: (a) the VH region comprises an amino acid sequence having at least 90%, 93%, 95%, 96%, 97%, 98%, or 99% identity to SEQ ID NO: 45, and the VL region comprises an amino acid sequence having at least 90%, 93%, 95%, 96%, 97%, 98%, or 99% identity to SEQ ID NO: 39; (b) the VH region comprises an amino acid sequence having at least 90%, 93%, 95%, 96%, 97%, 98%, or 99% identity to SEQ ID NO: 43, and the VL region comprises an amino acid sequence having at least 90%, 93%, 95%, 96%, 97%, 98%, or 99% identity to SEQ ID NO: 37; (c) the VH region comprises an amino acid sequence having at least 90%, 93%, 95%, 96%, 97%, 98%, or 99% identity to SEQ ID NO: 44, and the VL region comprises an amino acid sequence having at least 90%, 93%, 95%, 96%, 97%, 98%, or 99% identity to SEQ ID NO: 38; (d) the VH region comprises an amino acid sequence having at least 90%, 93%, 95%, 96%, 97%, 98%, or 99% identity to SEQ ID NO: 46, and the VL region comprises an amino acid sequence having at least 90%, 93%, 95%, 96%, 97%, 98%, or 99% identity to SEQ ID NO: 40; (e) the VH region comprises an amino acid sequence having at least 90%, 93%, 95%, 96%, 97%, 98%, or 99% identity to SEQ ID NO: 47, and the VL region comprises an amino acid sequence having at least 90%, 93%, 95%, 96%, 97%, 98%, or 99% identity to SEQ ID NO: 41; or (f) the VH region comprises an amino acid sequence having at least 90%, 93%, 95%, 96%, 97%, 98%, or 99% identity to SEQ ID NO: 48, and the VL region comprises an amino acid sequence having at least 90%, 93%, 95%, 96%, 97%, 98%, or 99% identity to SEQ ID NO: 42.

Embodiment 44.2 is the engineered cell of any one of embodiments 42, 42.1, 42.2, 43, 44, and 44.1, wherein: the VH region comprises an amino acid sequence having at least 90%, 93%, 95%, 96%, 97%, 98%, or 99% identity to SEQ ID NO: 45, and the VL region comprises an amino acid sequence having at least 90%, 93%, 95%, 96%, 97%, 98%, or 99% identity to SEQ ID NO: 39.

Embodiment 44.3 is the engineered cell of any one of embodiments 42, 42.1, 42.2, 43, 44, 44.1, and 44.2, wherein: (a) the VH region comprises the amino acid sequence of SEQ ID NO: 45, and the VL region comprises the amino acid sequence of SEQ ID NO: 39; (b) the VH region comprises the amino acid sequence of SEQ ID NO: 43, and the VL region comprises the amino acid sequence of SEQ ID NO: 37; (c) the VH region comprises the amino acid sequence of SEQ ID NO: 44, and the VL region comprises the amino acid sequence of SEQ ID NO: 38; (d) the VH region comprises the amino acid sequence of SEQ ID NO: 46, and the VL region comprises the amino acid sequence of SEQ ID NO: 40; (e) the VH region comprises the amino acid sequence of SEQ ID NO: 47, and the VL region comprises the amino acid sequence of SEQ ID NO: 41; or (f) the VH region comprises the amino acid sequence of SEQ ID NO: 48, and the VL region comprises the amino acid sequence of SEQ ID NO: 42.

Embodiment 44.4 is the engineered cell of any one of embodiments 42, 42.1, 42.2, 43, 44, and 44.1-44.3, wherein: the VH region comprises the amino acid sequence of SEQ ID NO: 45, and the VL region comprises the amino acid sequence of SEQ ID NO: 39.

Embodiment 45 is the engineered cell of any one of embodiments 42-44, 42.1, 42.2, and 44.1-44.4, wherein the antigen-binding protein comprises an amino acid sequence of any one of SEQ ID NOs: 25, 27, 29, 32, 34, and 36, or a sequence having at least 90%, 93%, 95%, 96%, 97%, 98%, or 99% identity to any one of SEQ ID NOs: 25, 27, 29, 32, 34, and 36.

Embodiment 46 is the engineered cell of any one of embodiments 42-45, 42.1, 42.2, and 44.1-44.4, wherein the antigen-binding protein comprises the amino acid sequence of SEQ ID NO: 25.

Embodiment 47 is the engineered cell of any one of embodiments 42-45, 42.1, 42.2, and 44.1-44.4, wherein the antigen-binding protein comprises the amino acid sequence of SEQ ID NO: 27.

Embodiment 48 is the engineered cell of any one of embodiments 42-47, 42.1, 42.2, and 44.1-44.4, wherein the antigen-binding protein comprises an amino acid sequence of SEQ ID NO: 25 or SEQ ID NO:27 and the costimulatory domain comprises the amino acid sequence of SEQ ID NO: 99.

Embodiment 49 is the engineered cell of any one of embodiments 42-47, 42.1, 42.2, and 44.1-44.4, wherein the antigen-binding protein comprises an amino acid sequence of SEQ ID NO: 25 or SEQ ID NO: 27 and the costimulatory domain comprises the amino acid sequence of SEQ ID NO: 101.

Embodiment 50 is the engineered cell of any one of embodiments 42-49, 42.1, 42.2, and 44.1-44.4, wherein the transmembrane domain comprises a CD8a transmembrane domain comprising an amino acid sequence of SEQ ID NO: 95.

Embodiment 51 is the engineered cell of any one of embodiments 42-49, 42.1, 42.2, and 44.1-44.4, wherein the transmembrane domain comprises a CD28 transmembrane domain comprising an amino acid sequence of SEQ ID NO: 93.

Embodiment 52 is the engineered cell of any one of embodiments 42-51, 42.1, 42.2, and 44.1-44.4, further comprising a hinge domain between the antigen binding protein and the transmembrane domain.

Embodiment 53 is the engineered cell of any one of embodiments 42-52, 42.1, 42.2, and 44.1-44.4, wherein the hinge domain is a CD8a hinge domain, or fragment thereof, comprising an amino acid sequence of SEQ ID NO: 89.

Embodiment 54 is the engineered cell of any one of embodiments 42-53, 42.1, 42.2, and 44.1-44.4, wherein the intracellular domain further comprises an activation domain.

Embodiment 55 is the engineered cell of any one of embodiments 42-54, 42.1, 42.2, and 44.1-44.4, wherein the activation domain is a CD3z activation domain comprising an amino acid sequence of SEQ ID NO: 103.

Embodiment 56 is the engineered cell of any one of embodiments 42-55, 42.1, 42.2, and 44.1-44.4, wherein the intracellular domain comprises a CD28 co-stimulatory domain comprising the amino acid sequence of SEQ ID NO: 99 and a CD3z activation domain comprising the amino acid sequence of SEQ ID NO: 103.

Embodiment 57 is the engineered cell of any one of embodiments 42-55, 42.1, 42.2, and 44.1-44.4, wherein the intracellular domain comprises a 41BB co-stimulatory domain comprising the amino acid sequence of SEQ ID NO: 101 and a CD3z activation domain comprising the amino acid sequence of SEQ ID NO: 103.

Embodiment 58 is the engineered cell of any one of embodiments 42-57, 42.1, 42.2, and 44.1-44.4, wherein: a. the antigen-binding protein comprises a sequence of SEQ ID NO: 32 and the costimulatory domain comprises a sequence of SEQ ID NO: 101; b. the antigen-binding protein comprises a sequence of SEQ ID NO: 32 and the costimulatory domain comprises a sequence of SEQ ID NO: 99; c. the antigen-binding protein comprises a sequence of SEQ ID NO: 25 and the costimulatory domain comprises a sequence of SEQ ID NO: 101; d. the antigen-binding protein comprises a sequence of SEQ ID NO: 25 and the costimulatory domain comprises a sequence of SEQ ID NO: 99; e. the antigen-binding protein comprises a sequence of SEQ ID NO: 27 and the costimulatory domain comprises a sequence of SEQ ID NO: 101; f. the antigen-binding protein comprises a sequence of SEQ ID NO: 27 and the costimulatory domain comprises a sequence of SEQ ID NO: 99; g. the antigen-binding protein comprises a sequence of SEQ ID NO: 36 and the costimulatory domain comprises a sequence of SEQ ID NO: 101; or h. the antigen-binding protein comprises a sequence of SEQ ID NO: 36 and the costimulatory domain comprises a sequence of SEQ ID NO: 99.

Embodiment 59 is the engineered cell of any one of embodiments 42-58, 42.1, 42.2, and 44.1-44.4, wherein the anti-CD70 CAR comprises an amino acid sequence that is at least 95% identical to the amino acid sequence of SEQ ID NO: 2, 4, 6, 8, 14, 16, 20, and 22.

Embodiment 60 is the engineered cell of any one of embodiments 42-59, 42.1, 42.2, and 44.1-44.4, wherein the anti-CD70 CAR comprises an amino acid sequence that is at least 95% identical to the amino acid sequence of SEQ ID NO: 4.

Embodiment 61 is the engineered cell of any one of embodiments 42-59, 42.1, 42.2, and 44.1-44.4, wherein the anti-CD70 CAR comprises an amino acid sequence that is at least 95% identical to the amino acid sequence of SEQ ID NO: 8.

Embodiment 62 is the engineered cell of any one of embodiments 42-59, 42.1, 42.2, and 44.1-44.4, wherein the anti-CD70 CAR comprises an amino acid sequence that is at least 95% identical to the amino acid sequence of SEQ ID NO: 2.

Embodiment 63 is the engineered cell of any one of embodiments 42-62, 42.1, 42.2, and 44.1-44.4, wherein the antigen binding protein is an scFv.

Embodiment 64 is the engineered cell of embodiment 63, wherein the scFv comprises a linker between the VH and VL regions.

Embodiment 65 is the engineered cell of embodiment 63 or 64, wherein the scFv comprises a glycine-serine linker between the VH and VL regions, optionally the linker comprising the sequence of SEQ ID NO: 940.

Embodiment 66 is a population of cells, wherein the population of cells comprises the engineered cell of any one of embodiments 42-65, 42.1, 42.2, and 44.1-44.4.

Embodiment 67 is the engineered cell or population of cells of any one of embodiments 42-66, 42.1, 42.2, and 44.1-44.4, further comprising reduced or eliminated surface expression of TGFBR2 relative to an unmodified cell.

Embodiment 68 is the engineered cell or population of cells of any one of embodiments 42-67, 42.1, 42.2, and 44.1-44.4, further comprising a genetic modification in the TGFBR2 gene

Embodiment 69 is the engineered cell or population of cells of embodiment 68, wherein the genetic modification comprises at least one nucleotide within the genomic coordinates: chr3: 30606864-30691614.

Embodiment 70 is the engineered cell or population of cells of embodiment 68 or 69, wherein the genetic modification is within the genomic coordinates chr3: 30674205-30674229.

Embodiment 71 is the engineered cell or population of cells of any one of embodiments 68-70, wherein the genetic modification comprises at least one nucleotide within the genomic coordinates targeted by a TGFBR2 guide RNA comprising a guide sequence of SEQ ID NO: 301.

Embodiment 72 is the engineered cell or population of cells of embodiment 68, wherein the genetic modification within the genomic coordinates chr3: 30671941-30671961.

Embodiment 73 is the engineered cell or population of cells of embodiment 68 or 72, wherein the genetic modification comprises at least one nucleotide within the genomic coordinates targeted by a TGFBR2 guide RNA comprising a guide sequence of SEQ ID NO: 302.

Embodiment 74 is the engineered cell or population of cells of any one of embodiments 42-73, 42.1, 42.2, and 44.1-44.4, further comprising reduced or eliminated surface expression of CD70 relative to an unmodified cell.

Embodiment 75 is the engineered cell or population of cells of any one of embodiments 42-74, 42.1, 42.2, and 44.1-44.4, further comprising a genetic modification in the CD70 gene.

Embodiment 76 is the engineered cell or population of cells of embodiment 75, wherein the genetic modification comprises at least one nucleotide within the genomic coordinates chr19: 6586002-6591018.

Embodiment 77 is the engineered cell or population of cells of embodiment 76, wherein the genetic modification is within the genomic coordinates: chr19: 6590121-6590145 or chr19: 6586268-6586292.

Embodiment 78 is the engineered cell or population of cells of embodiment 76 or 77, wherein the genetic modification comprises at least one nucleotide within the genomic coordinates targeted by a CD70 guide RNA comprising a guide sequence of SEQ ID NO: 310 or 311.

Embodiment 79 is the engineered cell or population of cells of embodiment 75, wherein the genetic modification is within the genomic coordinates: chr19: 6586028-6591018.

Embodiment 80 is the engineered cell or population of cells of embodiment 79, wherein the genetic modification comprises at least one nucleotide within the genomic coordinates: chr19: 6590998-6591018 or chr19: 6590991-6591011.

Embodiment 81 is the engineered cell or population of cells of embodiment 79 or 80, wherein the genetic modification comprises at least one nucleotide within the genomic coordinates targeted by a CD70 guide RNA comprising a guide sequence of SEQ ID NO: 312 or 313.

Embodiment 82 is the engineered cell or population of cells of any one of embodiments 42-81, 42.1, 42.2, and 44.1-44.4, further comprising reduced or eliminated surface expression of HLA-A relative to an unmodified cell.

Embodiment 83 is the engineered cell or population of cells of any one of embodiments 42-82, 42.1, 42.2, and 44.1-44.4, further comprising a genetic modification in the HLA-A gene.

Embodiment 84 is the engineered cell or population of cells of embodiment 83, wherein the genetic modification comprises at least one nucleotide within the genomic coordinates: (a) chr6: 29942854-29942913 and chr6: 29943518-29943619; and (b) chr6: 29942540-29945459.

Embodiment 85 is the engineered cell or population of cells of embodiment 83 or 84, wherein the genetic modification is within the genomic coordinates chosen from chr6: 29942891-29942915; and chr6: 29942609-29942633.

Embodiment 86 is the engineered cell or population of cells of any one of embodiments 83-85, wherein the genetic modification comprises at least one nucleotide within the genomic coordinates targeted by an HLA-A guide RNA comprising a guide sequence of SEQ ID NO: 403 or 404.

Embodiment 87 is the engineered cell or population of cells of any one of embodiments 42-86, 42.1, 42.2, and 44.1-44.4, which has reduced or eliminated surface expression of HLA-B relative to an unmodified cell.

Embodiment 88 is the engineered cell or population of cells of any one of embodiments 42-87, 42.1, 42.2, and 44.1-44.4, further comprising a genetic modification in the HLA-B gene.

Embodiment 89 is the engineered cell or population of cells of embodiment 88, wherein the genetic modification is within the genomic coordinates chosen from (a) chr6: 31354480-31357174 and (b) chr6: 31357084-31354647.

Embodiment 90 is the engineered cell of embodiment 88 or 89, wherein the genetic modification is within the genomic coordinates chosen from chr6: 31355222-31355246, chr6: 31355221-31355245, and chr6: 31355205-31355229.

Embodiment 91 is the engineered cell or population of cells of any one of embodiments 88-90, wherein the genetic modification comprises at least one nucleotide within the genomic coordinates targeted by an HLA-B guide RNA comprising a guide sequence of SEQ ID NO: 406, 405, or 407.

Embodiment 92 is the engineered cell or population of cells of any one of embodiments 42-91, 42.1, 42.2, and 44.1-44.4, comprising reduced or eliminated surface expression of TRAC relative to an unmodified cell.

Embodiment 93 is the engineered cell or population of cells of embodiment 42-92, 42.1, 42.2, and 44.1-44.4, comprising a genetic modification in the TRAC gene.

Embodiment 94 is the engineered cell or population of cells of embodiment 93, wherein the genetic modification comprises at least one nucleotide within the genomic coordinates chr14: 22547524-22547544.

Embodiment 95 is the engineered cell or population of cells of embodiment 93 or 94, wherein the genetic modification comprises at least one nucleotide within the genomic coordinates targeted by a TRAC guide RNA comprising a guide sequence of SEQ ID NO: 413.

Embodiment 96 is the engineered cell or population of cells of any one of embodiments 42-95, 42.1, 42.2, and 44.1-44.4, which has reduced or eliminated surface expression of MHC class II relative to an unmodified cell.

Embodiment 97 is the engineered cell or population of cells of any one of embodiments 42-96, 42.1, 42.2, and 44.1-44.4, further comprising a genetic modification in the CIITA gene.

Embodiment 98 is the engineered cell or population of cells of embodiment 97, wherein the genetic modification is within the genetic coordinates chosen from: (a) chr16: 10877363-10907788 and (b) chr16: 10906515-10908136.

Embodiment 99 is the engineered cell or population of cells of embodiment 97 or 98, wherein the genetic modification comprises at least one nucleotide within the genomic coordinates is chr16: 10906643-10906667 or chr16: 10907504-10907528.

Embodiment 100 is the engineered cell or population of cells of any one of embodiments 97-99, wherein the genetic modification comprises at least one nucleotide within the genomic coordinates targeted by a CIITA guide RNA comprising a guide sequence of SEQ ID NO: 402 or 401.

Embodiment 101 is the engineered cell or population of cells of any one of embodiments 68-100, wherein the genetic modification comprises an indel, a C to T substitution, or an A to G substitution within the genomic coordinates.

Embodiment 102 is the engineered cell or population of cells of any one of embodiments 68-101, wherein the genetic modification comprises a C to T substitution within the genomic coordinates.

Embodiment 103 is an engineered cell comprising a genetic modification in the HLA-A gene, a modified TRAC gene, a genetic modification in the CIITA gene, a genetic modification in the TGFBR2 gene, and/or a genetic modification in the CD70 gene, wherein the engineered cell expresses an anti-CD70 chimeric antigen receptor (CAR) or comprises a nucleic acid encoding an anti-CD70 CAR.

Embodiment 104 is an engineered cell comprising a genetic modification in the HLA-A gene, a genetic modification in the HLA-B gene, a genetic modification in the TRAC gene, a genetic modification in the CIITA gene, a genetic modification in the TGFBR2 gene, and/or a genetic modification in the CD70 gene, wherein the engineered cell expresses an anti-CD70 chimeric antigen receptor (CAR) or comprises a nucleic acid encoding an anti-CD70 CAR.

Embodiment 105 is the engineered cell of embodiment 103 or 104, wherein the genetic modification in TGFBR2 comprises at least one nucleotide within the genomic coordinates: chr3: 30606864-30691614.

Embodiment 106 is the engineered cell of embodiment 103 or 104, wherein the genetic modification in TGFBR2 is within the genomic coordinates: chr3: 30606891-30691605.

Embodiment 107 is the engineered cell of any one of embodiments 103-106, wherein the genetic modification in TGFBR2 is within the genomic coordinates chr3: 30674205-30674229.

Embodiment 108 is the engineered cell of any one of embodiments 103-107, wherein the genetic modification in TGFBR2 comprises at least one nucleotide within the genomic coordinates targeted by a TGFBR2 guide RNA comprising a guide sequence of SEQ ID NO: 301.

Embodiment 109 is the engineered cell of any one of embodiments 103-106, wherein the genetic modification in TGFBR2 is within the genomic coordinates: chr3: 30671941-30671961.

Embodiment 110 is the engineered cell of any one of embodiments 103-106 and 109, wherein the genetic modification in TGFBR2 comprises at least one nucleotide within the genomic coordinates targeted by a TGFBR2 guide RNA comprising a guide sequence of SEQ ID NO: 302.

Embodiment 111 is the engineered cell of any one of embodiments 103-110, wherein the genetic modification in CD70 is within the genetic coordinates chr19: 6586002-6591015.

Embodiment 112 is the engineered cell of any one of embodiments 103-111, wherein the genetic modification in CD70 is within the genetic coordinates chr19: 6590121-6590145 or chr19: 6586268-6586292.

Embodiment 113 is the engineered cell of any one of embodiments 103-112, wherein the genetic modification in CD70 comprises at least one nucleotide within the genomic coordinates targeted by a CD70 guide RNA comprising a guide sequence of SEQ ID NO: 310 or 311.

Embodiment 114 is the engineered cell of any one of embodiments 103-110, wherein the genetic modification in CD70 is within the genomic coordinates: chr19: 6586028-6591018.

Embodiment 115 is the engineered cell of any one of embodiments 103-110 and 114, wherein the genetic modification in CD70 comprises at least one nucleotide within the genomic coordinates chr19: 6590998-6591018 or chr19: 6590991-6591011, optionally within the genomic coordinates chr19: 6590998-6591018.

Embodiment 116 is the engineered cell of any one of embodiments 103-110, 114, and 115, wherein the genetic modification in CD70 comprises at least one nucleotide within the genomic coordinates targeted by a CD70 guide RNA comprising a guide sequence of SEQ ID NO: 312 or 313, optionally SEQ ID NO: 312.

Embodiment 117 is the engineered cell of any one of embodiments 103-116, wherein the genetic modification in HLA-A is within the genetic coordinates chr6: 29942540-29945459.

Embodiment 118 is the engineered cell of any one of embodiments 103-117, wherein the genetic modification in HLA-A is within the genomic coordinates chosen from chr6: 29942891-29942915; and chr6: 29942609-29942633.

Embodiment 119 is the engineered cell of any one of embodiments 103-118, wherein the genetic modification in HLA-A comprises at least one nucleotide within the genomic coordinates targeted by an HLA-A guide RNA comprising a guide sequence of SEQ ID NO: 403 or 404.

Embodiment 120 is the engineered cell of any one of embodiments 104-119, wherein the genetic modification in HLA-B comprises at least one nucleotide within the genetic coordinates chr6: 31354480-31357174.

Embodiment 121 is the engineered cell of any one of embodiments 104-120, wherein the genetic modification in HLA-B is within the genomic coordinates: chr6: 31355222-31355246, chr6: 31355221-31355245, or chr6: 31355205-31355229.

Embodiment 122 is the engineered cell of any one of embodiments 104-121, wherein the genetic modification in HLA-B comprises at least one nucleotide within the genomic coordinates targeted by an HLA-B guide RNA comprising a guide sequence of SEQ ID NO: 405-407.

Embodiment 123 is the engineered cell of any one of embodiments 104-122, wherein the genetic modification in CIITA is within the genetic coordinates chosen from: (a) chr16: 10877363-10907788 and (b) chr16: 10906515-10908136.

Embodiment 124 is the engineered cell of any one of embodiments 104-123, wherein the genetic modification in CIITA is within the genetic coordinates: chr16: 10906643-10906667 or chr16: 10907504-10907528.

Embodiment 125 is the engineered cell of any one of embodiments 103-124, wherein the genetic modification in CIITA comprises at least one nucleotide within the genomic coordinates targeted by a CIITA guide RNA comprising a guide sequence of SEQ ID NO: 402 or 401.

Embodiment 126 is the engineered cell of any one of embodiments 103-125, wherein the genetic modification in TRAC is within the genomic coordinates chr14: 22547524-22547544.

Embodiment 127 is the engineered cell of any one of embodiments 103-126, wherein the genetic modification in TRAC comprises at least one nucleotide within the genomic coordinates targeted by a TRAC guide RNA comprising a guide sequence of SEQ ID NO: 413.

Embodiment 128 is the engineered cell of any one of embodiments 103-127, wherein the genetic modification comprises an indel, a C to T substitution, or an A to G substitution within the genomic coordinates.

Embodiment 129 is the engineered cell of any one of embodiments 103-128, wherein the genetic modification comprises a C to T substitution within the genomic coordinates.

Embodiment 130 is the engineered cell or population of cells of any one of embodiments 42-127, 42.1, 42.2, and 44.1-44.4, wherein the cell is homozygous for HLA-C.

Embodiment 131 is the engineered cell or population of cells of any one of embodiments 42-128, 42.1, 42.2, and 44.1-44.4, wherein the cell is homozygous for HLA-B and HLA-C.

Embodiment 132 is a population of cells comprising the engineered cells of any one of embodiments 103-131.

Embodiment 133 is a pharmaceutical composition comprising the engineered cell or population of cells of any one of embodiments 42-132, 42.1, 42.2, and 44.1-44.4.

Embodiment 134 is the engineered cell, population of cells, or pharmaceutical composition of any one of embodiments 42-133, 42.1, 42.2, and 44.1-44.4, wherein the genetic modification comprises at least 2, at least 3, at least 4, at least 5, at least 6, at least 7, at least 8, at least 9, or at least 10 contiguous nucleotides within the genomic coordinates.

Embodiment 135 is the engineered cell, population of cells, or pharmaceutical composition of any one of embodiments 42-134, 42.1, 42.2, and 44.1-44.4, wherein the genetic modification comprises an indel.

Embodiment 136 is the engineered cell, population of cells, or pharmaceutical composition of any one of embodiments 42-135, 42.1, 42.2, and 44.1-44.4, wherein the genetic modification comprises an insertion of a heterologous coding sequence.

Embodiment 137 is the engineered cell, population of cells, or pharmaceutical composition of any one of embodiments 42-136, 42.1, 42.2, and 44.1-44.4, wherein the genetic modification comprises at least one A to G substitution within the genomic coordinates.

Embodiment 138 is the engineered cell, population of cells, or pharmaceutical composition of any one of embodiments 42-137, 42.1, 42.2, and 44.1-44.4, wherein the genetic modification comprises at least one C to T substitution within the genomic coordinates.

Embodiment 139 is the engineered cell, population of cells, or pharmaceutical composition of any one of embodiments 42-138, 42.1, 42.2, and 44.1-44.4, wherein the cell has reduced expression of TRAC protein on the surface of the cell.

Embodiment 140 is the engineered cell, population of cells, or pharmaceutical composition of any one of embodiments 42-139, 42.1, 42.2, and 44.1-44.4, wherein the cell has a genetic modification in the CIITA gene.

Embodiment 141 is the engineered cell, population of cells, or pharmaceutical composition of any one of embodiments 42-140, 42.1, 42.2, and 44.1-44.4, wherein the cell has reduced expression of MHC class II molecules on the surface of the cell.

Embodiment 142 is the engineered cell, population of cells, or pharmaceutical composition of any one of embodiments 42-141, 42.1, 42.2, and 44.1-44.4, wherein the engineered cell is an immune cell.

Embodiment 143 is the engineered cell, population of cells, or pharmaceutical composition of any one of embodiments 42-142, 42.1, 42.2, and 44.1-44.4, wherein the cell is an NK cell.

Embodiment 144 is the engineered cell, population of cells, or pharmaceutical composition of any one of embodiments 42-142, 42.1, 42.2, and 44.1-44.4, wherein the cell is a T-cell.

Embodiment 145 is the engineered cell, population of cells, or pharmaceutical composition of embodiment 144, wherein the T-cell is a CD4+ T-cell.

Embodiment 146 is the engineered cell, population of cells, or pharmaceutical composition of embodiment 144, wherein the T-cell is a CD8+ T-cell.

Embodiment 147 is the engineered cell, population of cells, or pharmaceutical compositions of embodiment 144, wherein the T-cell has a T memory stem cell (Tscm) phenotype.

Embodiment 148 is the engineered cell, population of cells, or pharmaceutical composition of any one of embodiments 42-141, 42.1, 42.2, and 44.1-44.4, wherein the engineered cell is a stem cell.

Embodiment 149 is the engineered cell, population of cells, or pharmaceutical composition of any one of embodiments 42-148, 42.1, 42.2, and 44.1-44.4, wherein the engineered cell is a primary cell.

Embodiment 150 is the engineered cell, population of cells, or pharmaceutical composition of any one of embodiments 42-149, 42.1, 42.2, and 44.1-44.4, wherein the cells are engineered with a genomic editing system.

Embodiment 151 is the engineered cell, population of cells, or pharmaceutical composition of any one of embodiments 42-150, 42.1, 42.2, and 44.1-44.4, wherein the genomic editing system comprises an RNA-guided DNA-binding agent or a nucleic acid encoding an RNA-guided DNA-binding agent.

Embodiment 152 is the engineered cell, population of cells, or pharmaceutical composition of embodiment 151, wherein the RNA-guided DNA-binding agent or the RNA-guided DNA-binding agent encoded by the nucleic acid is S. pyogenes Cas9 (SpyCas9).

Embodiment 153 is the engineered cell, population of cells, or pharmaceutical composition of embodiment 151, wherein the RNA-guided DNA-binding agent or the RNA-guided DNA-binding agent encoded by the nucleic acid is N. meningitidis Cas9 (NmeCas9).

Embodiment 154 is the engineered cell, population of cells, or pharmaceutical composition of any one of embodiments 151-153, wherein the RNA-guided DNA-binding agent or the RNA-guided DNA-binding agent encoded by the nucleic acid has double-stranded endonuclease activity.

Embodiment 155 is the engineered cell, population of cells, or pharmaceutical composition of any one of embodiments 151-153, wherein the RNA-guided DNA-binding agent or the RNA-guided DNA-binding agent encoded by the nucleic acid has nickase activity.

Embodiment 156 is the engineered cell, population of cells, or pharmaceutical composition of any one of embodiments 151-153, wherein the RNA-guided DNA-binding agent or the RNA-guided DNA-binding agent encoded by the nucleic acid comprises a dCas9 DNA-binding domain.

Embodiment 157 is the engineered cell, population of cells, or pharmaceutical composition of any one of embodiments 151-156, wherein the RNA-guided DNA-binding agent or nucleic acid encoding the RNA-guided DNA-binding agent is a A to G base editor.

Embodiment 158 is the engineered cell, population of cells, or pharmaceutical composition of any one of embodiments 151-156, wherein the RNA-guided DNA-binding agent or nucleic acid encoding the RNA-guided DNA-binding agent is a C to T base editor.

Embodiment 159 is the engineered cell, population of cells, or pharmaceutical composition of any one of embodiments 71-158, wherein the guide RNA is provided to the cell in a vector.

Embodiment 160 is the engineered cell, population of cells, or pharmaceutical composition of any one of embodiments 71-159, wherein the RNA-guided DNA-binding agent is provided to the cell in a vector, optionally in the same vector as the guide RNA.

Embodiment 161 is the engineered cell, population of cells, or pharmaceutical composition of any one of embodiments 42-160, 42.1, 42.2, and 44.1-44.4, wherein the nucleic acid encoding the anti-CD70 CAR is provided to the cell in an expression vector.

Embodiment 162 is the engineered cell, population of cells, or pharmaceutical composition of embodiment 161, wherein the expression vector is a viral vector.

Embodiment 163 is the engineered cell, population of cells, or pharmaceutical composition of embodiment 162, wherein the expression vector comprises an AAV vector.

Embodiment 164 is the engineered cell, population of cells, or pharmaceutical composition of embodiment 163, wherein the expression vector comprises SEQ ID NO: 106.

Embodiment 165 is the engineered cell, population of cells, or pharmaceutical composition of embodiment 163, wherein the engineered cell comprises SEQ ID NO: 107.

Embodiment 166 is the engineered cell, population of cells, or pharmaceutical composition of embodiment 161, wherein the expression vector is a non-viral vector.

Embodiment 167 is the engineered cell, population of cells, or pharmaceutical composition of any one of embodiments 71-166, wherein the guide RNA is provided to the cell in a lipid nanoparticle (LNP), optionally in the same LNP an RNA-guided DNA-binding agent is provided.

Embodiment 168 is the engineered cell, population of cells, or pharmaceutical composition of any one of embodiments 42-167, 42.1, 42.2, and 44.1-44.4, wherein the nucleic acid encoding the anti-CD70 CAR is provided to the cell in a lipid nanoparticle (LNP).

Embodiment 169 is the engineered cell, population of cells, or pharmaceutical composition of any one of embodiments 71-168, wherein the guide RNA is a single guide RNA.

Embodiment 170 is the engineered cell, population of cells, or pharmaceutical composition of any one of embodiments 71-169, wherein the guide RNA comprises a 5′ end modification or a 3′ end modification.

Embodiment 171 is the engineered cell, population of cells, or pharmaceutical composition of any one of embodiments 71-170, wherein the guide RNA comprises a sequence of SEQ ID NO: 712 or 722, or a sequence that is at least 90%, 95%, 98%, 99%, identical to SEQ ID NO: 712 or 731.

Embodiment 172 is the engineered cell, population of cells, or pharmaceutical composition of any one of embodiments 71-170, wherein the guide RNA comprises a sequence of SEQ ID NO: 713 or 723, or a sequence that is at least 90%, 95%, 98%, 99%, identical to SEQ ID NO: 713 or 723.

Embodiment 173 is the engineered cell, population of cells, or pharmaceutical composition of any one of embodiments 71-170, wherein the guide RNA comprises a sequence of SEQ ID NO: 620 or 658, or a sequence that is at least 90%, 95%, 98%, 99%, identical to SEQ ID NO: 620 or 658.

Embodiment 174 is the engineered cell, population of cells, or pharmaceutical composition of any one of embodiments 71-170, wherein the guide RNA comprises a sequence of SEQ ID NO: 641 or 669, or a sequence that is at least 90%, 95%, 98%, 99%, identical to SEQ ID NO: 641 or 669.

Embodiment 175 is a method of making an engineered cell comprising contacting a cell with: a. the nucleic acid of any one of embodiments 25-32, or the mRNA of embodiment 33, or the expression vector of embodiment 34; and b. at least one genomic editing tool comprising a genomic editor and at least one guide RNA, wherein the at least one guide RNA targets a genomic locus chosen from the HLA-A, HLA-B, TRAC, CIITA, TGFBR2, and CD70 locus.

Embodiment 176 is a method of making an engineered cell comprising an anti-CD70 CAR, comprising a. providing an engineered cell which has reduced or eliminated surface expression of one or both of TGFBR2 and CD70 relative to an unmodified cell; and b. contacting the cell with the nucleic acid of any one of embodiments 25-32, or the mRNA of embodiment 33, or the expression vector of embodiment 34.

Embodiment 177 is a method of making an engineered cell comprising an anti-CD70 CAR, comprising a. providing an engineered cell which has reduced or eliminated surface expression of one or more of HLA-A, HLA-B, MHC Class II, TRAC, TGFBR2, and CD70, relative to an unmodified cell; and b. contacting the cell with the nucleic acid of any one of embodiments 25-32, or the mRNA of embodiment 33, or the expression vector of embodiment 34.

Embodiment 178 is a method of making an engineered cell comprising an anti-CD70 CAR, comprising: (a) contacting the cell with a first group of lipid nanoparticles (LNPs) comprising a LNP comprising a UGI and at least one LNP comprising a base editor and at least one guide RNA that is cognate to the base editor and targets a genomic locus chosen from the HLA-A, HLA-B, TRAC, CIITA, TGFBR2, and CD70 locus; and (b) contacting a cell with i. a second group of LNPs comprising a LNP comprising a UGI and at least one LNP comprising a base editor and at least one guide RNA that is cognate to the base editor and targets a genomic locus chosen from the HLA-A, HLA-B, TRAC, CIITA, TGFBR2, and CD70 locus that are different from the at least one guide RNA contained in the first group of LNPs of the step (a); and ii. at least one LNP comprising an RNA-guided cleavase and at least one gRNA that is cognate to the RNA-guided cleavase and targets the TRAC locus; and iii. a nucleic acid encoding an anti-CD70 CAR for insertion into an editing site (e.g., a double strand break) at the TRAC locus.

Embodiment 179 is a method of making an engineered cell comprising an anti-CD70 CAR, comprising: (a) contacting a cell with a first group of lipid nanoparticles (LNPs) comprising a first LNP comprising a base editor and a gRNA that targets the HLA-A locus; a second LNP comprising a base editor and comprising a gRNA that targets the HLA-B locus, a third LNP comprising a base editor and comprising a gRNA that targets the CIITA locus, and a fourth LNP comprising a uracil glycosylase inhibitor (UGI); (b) contacting a cell with (i) a second group of LNPs comprising a fifth LNP comprising a base editor and comprising a gRNA that targets the TGFBR2 locus; a sixth LNP comprising a base editor and comprising a gRNA that targets the CD70 locus; (ii) a seventh LNP comprising an RNA-guided DNA cleavase and a gRNA that is cognate to the RNA-guide DNA cleavase and targets the TRAC locus, and an eight lipid LNP comprising a UGI; and (iii) a nucleic acid encoding an anti-CD70 CAR for insertion into an editing site (e.g., a double strand break) at the TRAC locus.

Embodiment 180 is the method of embodiment 178 or 179, wherein the RNA-guided cleavase comprises an S. pyogenes (Spy) Cas9 cleavase, and the base editor comprises an N. meningitidis (Nme) Cas9 nickase.

Embodiment 181 is a method of administering the engineered cell, population of cells, or pharmaceutical composition of any one of embodiments 42-174, 42.1, 42.2, and 44.1-44.4 to a subject in need thereof.

Embodiment 182 is a method of administering the engineered cell, population of cells, or pharmaceutical composition of any one of embodiments 42-174, 42.1, 42.2, and 44.1-44.4 to a subject as an adoptive cell transfer (ACT) therapy.

Embodiment 183 is a method of treating a disease or disorder, comprising administering the engineered cell, population of cells, or pharmaceutical composition of any one of embodiments 42-174, 42.1, 42.2, and 44.1-44.4 to a subject in need thereof.

Embodiment 183.1 is the method of any one of embodiments 181-183, wherein the engineered cell is allogeneic to the subject.

Embodiment 184 is the engineered cell, population of cells, composition, or method of any one of embodiments 42-174, 42.1, 42.2, and 44.1-44.4, for use in administration to a subject as an adoptive cell transfer (ACT) therapy.

Embodiment 185 is the engineered cell, population of cells, pharmaceutical composition, or method of any one of embodiments 42-174, 42.1, 42.2, and 44.1-44.4, for use in treating a subject having a cancer.

Embodiment 186 is the engineered cell, population of cells, or pharmaceutical composition of any one of embodiments 42-174, 42.1, 42.2, and 44.1-44.4, for use in treating a subject having an infectious disease.

Embodiment 187 is the engineered cell, population of cells, or pharmaceutical composition of any one of embodiments 42-174, 42.1, 42.2, and 44.1-44.4, for use in treating a subject having an autoimmune disease.

Embodiment 188 is use of the engineered cell, population of cells, pharmaceutical composition of any one of embodiments 42-174, 42.1, 42.2, and 44.1-44.4, for the manufacture of a medicament for the treatment of a subject having a cancer, an infectious disease, or an autoimmune disease.

Embodiment 189 is an engineered cell comprising a genetic modification in the HLA-A gene, a genetic modification in the HLA-B gene, a genetic modification in the TRAC gene, a genetic modification in the CIITA gene, a genetic modification in the TGFBR2 gene, and/or a genetic modification in the CD70 gene, wherein the genetic modification in the HLA-A gene is within the genomic coordinates chr6: 29942891-29942915; wherein the genetic modification in the HLA-B gene is within the genomic coordinates chr6: 31355222-31355246, chr6: 31355221-31355245, or chr6: 31355205-31355229; wherein the genetic modification in the TRAC gene is within the genomic coordinates chr14: 22547524-22547544; wherein the genetic modification in the CIITA gene is within the genomic coordinates chr16: 10907504-10907528; wherein the genetic modification in the TGFBR2 gene is within the genomic coordinates chr3: 30674205-30674229 or chr3: 30671941-30671961; and wherein the genetic modification in the CD70 gene is within the genomic coordinates chr19: 6590121-6590145 or chr19: 6590998-6591018, wherein the engineered cell comprises an anti-CD70 chimeric antigen receptor (CAR) or comprises a nucleic acid encoding an anti-CD70 CAR.

Embodiment 190 is an engineered cell comprising a genetic modification in the HLA-A gene, a genetic modification in the HLA-B gene, a genetic modification in the TRAC gene, a genetic modification in the CIITA gene, a genetic modification in the TGFBR2 gene, and/or a genetic modification in the CD70 gene, wherein the genetic modification in the HLA-A gene is within the genomic coordinates chr6: 29942891-29942915; wherein the genetic modification in the HLA-B gene is within the genomic coordinates chr6: 31355222-31355246; wherein the genetic modification in the TRAC gene is within the genomic coordinates chr14: 22547524-22547544; wherein the genetic modification in the CIITA gene is within the genomic coordinates chr16: 10906643-10906667; wherein the genetic modification in the TGFBR2 gene is within the genomic coordinates chr3: 30674205-30674229; and wherein the genetic modification in the CD70 gene is within the genomic coordinates chr19: 6590121-6590145, wherein the engineered cell comprises an anti-CD70 chimeric antigen receptor (CAR) or comprises a nucleic acid encoding an anti-CD70 CAR.

Embodiment 191 is the engineered cell of embodiment 189 or 190, wherein the CAR comprises the amino acid sequence of SEQ ID NO: 4, or wherein the nucleic acid encoding the CAR comprises the nucleic acid sequence of SEQ ID NO: 3 or 106.

Embodiment 192 is an engineered human T cell comprising multiple genetic modifications and an anti-CD70 chimeric antigen receptor (CAR), wherein (a) the engineered human T cell comprises a genetic modification in the HLA-A gene and reduced or eliminated surface expression of HLA-A relative to an unmodified cell, a genetic modification in the HLA-B gene and reduced or eliminated surface expression of HLA-B relative to an unmodified cell, a genetic modification in the CIITA gene and reduced or eliminated surface expression of MHC class II relative to an unmodified cell, a genetic modification in the TGFBR2 gene and reduced or eliminated surface expression of TGFBR2 relative to an unmodified cell, and a genetic modification in the CD70 gene and reduced or eliminated surface expression of CD70 relative to an unmodified cell, and (b) the anti-CD70 CAR comprises an antigen-binding protein, or a fragment thereof, that binds to CD70, wherein the antigen-binding protein comprises a heavy chain variable (VH) region comprising a complementarity determining region 1 (VH CDR1) comprising the amino acid sequence of SEQ ID NO: 73, a VH CDR2 comprising the amino acid sequence of SEQ ID NO: 74, and a VH CDR3 comprising the amino acid sequence of SEQ ID NO: 75, and comprises a light chain variable (VL) region comprising a complementarity determining region 1 (VL CDR1) comprising the amino acid sequence of SEQ ID NO: 55, a VL CDR2 comprising the amino acid sequence of SEQ ID NO: 56, and a VL CDR3 comprising the amino acid sequence of SEQ ID NO: 57, wherein the anti-CD70 CAR is inserted into the TRAC locus.

Embodiment 193 is an engineered human T cell comprising multiple genetic modifications and an anti-CD70 chimeric antigen receptor (CAR), wherein (a) the engineered human T cell comprises a genetic modification in the HLA-A gene within the genomic coordinates chr6: 29942891-29942915, a genetic modification in the HLA-B gene within the genomic coordinates chr6: 31355222-31355246, a genetic modification in the CIITA gene within the genomic coordinates chr16: 10906643-10906667, a genetic modification in the TGFBR2 gene within the genomic coordinates chr3: 30674205-30674229, and a genetic modification in the CD70 gene within the genomic coordinates chr19: 6590121-6590145, and (b) the anti-CD70 CAR comprises an antigen-binding protein, or a fragment thereof, that binds to CD70, wherein the antigen-binding protein comprises a heavy chain variable (VH) region comprising a complementarity determining region 1 (VH CDR1) comprising the amino acid sequence of SEQ ID NO: 73, a VH CDR2 comprising the amino acid sequence of SEQ ID NO: 74, and a VH CDR3 comprising the amino acid sequence of SEQ ID NO: 75, and comprises a light chain variable (VL) region comprising a complementarity determining region 1 (VL CDR1) comprising the amino acid sequence of SEQ ID NO: 55, a VL CDR2 comprising the amino acid sequence of SEQ ID NO: 56, and a VL CDR3 comprising the amino acid sequence of SEQ ID NO: 57, wherein the anti-CD70 CAR is inserted into the TRAC locus.

Embodiment 194 is an engineered human T cell comprising multiple genetic modifications and an anti-CD70 chimeric antigen receptor (CAR), wherein (a) the engineered human T cell comprises a genetic modification in the HLA-A gene within the genomic coordinates chr6: 29942891-29942915, a genetic modification in the HLA-B gene within the genomic coordinates chr6: 31355222-31355246, a genetic modification in the CIITA gene within the genomic coordinates chr16: 10906643-10906667, a genetic modification in the TGFBR2 gene within the genomic coordinates chr3: 30674205-30674229, and a genetic modification in the CD70 gene within the genomic coordinates chr19: 6590121-6590145, and (b) the anti-CD70 CAR comprises an antigen-binding protein, or a fragment thereof, that binds to CD70, wherein the antigen-binding protein comprises a heavy chain variable (VH) region comprising a complementarity determining region 1 (VH CDR1) comprising the amino acid sequence of SEQ ID NO: 73, a VH CDR2 comprising the amino acid sequence of SEQ ID NO: 74, and a VH CDR3 comprising the amino acid sequence of SEQ ID NO: 75, and comprises a light chain variable (VL) region comprising a complementarity determining region 1 (VL CDR1) comprising the amino acid sequence of SEQ ID NO: 55, a VL CDR2 comprising the amino acid sequence of SEQ ID NO: 56, and a VL CDR3 comprising the amino acid sequence of SEQ ID NO: 57, wherein the anti-CD70 CAR is inserted into the TRAC locus within the genomic coordinates chr14: 22547524-22547544.

Embodiment 195 is the engineered human T cell of any one of embodiments 192-194, wherein the VH region comprises the amino acid sequence of SEQ ID NO: 45, and the VL region comprises the amino acid sequence of SEQ ID NO: 39.

Embodiment 196 is the engineered human T cell of any one of embodiments 192-195, wherein the anti-CD70 CAR comprises the amino acid sequence of SEQ ID NO: 4.

Embodiment 197 is the engineered human T cell of any one of embodiments 192-196, wherein the engineered human T cell is a CD4+ or CD8+ T cell.

Embodiment 198 is the engineered human T cell of any one of embodiments 192-197, wherein the engineered human T cell is homozygous for HLA-C, optionally wherein the engineered human T cell is homozygous for HLA-B and for HLA-C.

Embodiment 199 is a pharmaceutical composition comprising a population of T cells, wherein the population of T cells comprises CD4+ T cells and/or CD8+ T cells, which comprise an engineered human T cell comprising multiple genetic modifications and an anti-CD70 chimeric antigen receptor (CAR), wherein (a) the engineered human T cell comprises a genetic modification in the HLA-A gene and reduced or eliminated surface expression of HLA-A relative to an unmodified cell, a genetic modification in the HLA-B gene and reduced or eliminated surface expression of HLA-B relative to an unmodified cell, a genetic modification in the CIITA gene and reduced or eliminated surface expression of MHC class II relative to an unmodified cell, a genetic modification in the TGFBR2 gene and reduced or eliminated surface expression of TGFBR2 relative to an unmodified cell, and a genetic modification in the CD70 gene and reduced or eliminated surface expression of CD70 relative to an unmodified cell, and (b) the anti-CD70 CAR comprises an antigen-binding protein, or a fragment thereof, that binds to CD70, wherein the antigen-binding protein comprises a heavy chain variable (VH) region comprising a complementarity determining region 1 (VH CDR1) comprising the amino acid sequence of SEQ ID NO: 73, a VH CDR2 comprising the amino acid sequence of SEQ ID NO: 74, and a VH CDR3 comprising the amino acid sequence of SEQ ID NO: 75, and comprises a light chain variable (VL) region comprising a complementarity determining region 1 (VL CDR1) comprising the amino acid sequence of SEQ ID NO: 55, a VL CDR2 comprising the amino acid sequence of SEQ ID NO: 56, and a VL CDR3 comprising the amino acid sequence of SEQ ID NO: 57, wherein the anti-CD70 CAR is inserted into the TRAC locus.

Embodiment 200 is a pharmaceutical composition comprising a population of T cells, wherein the population of T cells comprises CD4+ T cells and/or CD8+ T cells, which comprise an engineered human T cell comprising multiple genetic modifications and an anti-CD70 chimeric antigen receptor (CAR), wherein (a) the engineered human T cell comprises a genetic modification in the HLA-A gene within the genomic coordinates chr6: 29942891-29942915, a genetic modification in the HLA-B gene within the genomic coordinates chr6: 31355222-31355246, a genetic modification in the CIITA gene within the genomic coordinates chr16: 10906643-10906667, a genetic modification in the TGFBR2 gene within the genomic coordinates chr3: 30674205-30674229, and a genetic modification in the CD70 gene within the genomic coordinates chr19: 6590121-6590145, and (b) the anti-CD70 CAR comprises an antigen-binding protein, or a fragment thereof, that binds to CD70, wherein the antigen-binding protein comprises a heavy chain variable (VH) region comprising a complementarity determining region 1 (VH CDR1) comprising the amino acid sequence of SEQ ID NO: 73, a VH CDR2 comprising the amino acid sequence of SEQ ID NO: 74, and a VH CDR3 comprising the amino acid sequence of SEQ ID NO: 75, and comprises a light chain variable (VL) region comprising a complementarity determining region 1 (VL CDR1) comprising the amino acid sequence of SEQ ID NO: 55, a VL CDR2 comprising the amino acid sequence of SEQ ID NO: 56, and a VL CDR3 comprising the amino acid sequence of SEQ ID NO: 57, wherein the anti-CD70 CAR is inserted into the TRAC locus.

Embodiment 201 is a pharmaceutical composition comprising a population of T cells, wherein the population of T cells comprises CD4+ T cells and/or CD8+ T cells, which comprise an engineered human T cell comprising multiple genetic modifications and an anti-CD70 chimeric antigen receptor (CAR), wherein (a) the engineered human T cell comprises a genetic modification in the HLA-A gene within the genomic coordinates chr6: 29942891-29942915, a genetic modification in the HLA-B gene within the genomic coordinates chr6: 31355222-31355246, a genetic modification in the CIITA gene within the genomic coordinates chr16: 10906643-10906667, a genetic modification in the TGFBR2 gene within the genomic coordinates chr3: 30674205-30674229, and a genetic modification in the CD70 gene within the genomic coordinates chr19: 6590121-6590145, and (b) the anti-CD70 CAR comprises an antigen-binding protein, or a fragment thereof, that binds to CD70, wherein the antigen-binding protein comprises a heavy chain variable (VH) region comprising a complementarity determining region 1 (VH CDR1) comprising the amino acid sequence of SEQ ID NO: 73, a VH CDR2 comprising the amino acid sequence of SEQ ID NO: 74, and a VH CDR3 comprising the amino acid sequence of SEQ ID NO: 75, and comprises a light chain variable (VL) region comprising a complementarity determining region 1 (VL CDR1) comprising the amino acid sequence of SEQ ID NO: 55, a VL CDR2 comprising the amino acid sequence of SEQ ID NO: 56, and a VL CDR3 comprising the amino acid sequence of SEQ ID NO: 57, wherein the anti-CD70 CAR is inserted into the TRAC locus within the genomic coordinates chr14: 22547524-22547544.

Embodiment 202 is the pharmaceutical composition of any one of embodiments 199-201, wherein the VH region comprises the amino acid sequence of SEQ ID NO: 45, and the VL region comprises the amino acid sequence of SEQ ID NO: 39.

Embodiment 203 is the pharmaceutical composition of any one of embodiments 199-202, wherein the anti-CD70 CAR comprises the amino acid sequence of SEQ ID NO: 4.

Embodiment 204 is the pharmaceutical composition of any one of embodiments 199-203, wherein the engineered human T cell is homozygous for HLA-C, optionally wherein the engineered human T cell is homozygous for HLA-B and for HLA-C.

Embodiment 205 is a method of administering the engineered human T cell or pharmaceutical composition of any one of embodiments 192-204 to a subject in need thereof, or to a subject as an adoptive cell transfer (ACT) therapy.

Embodiment 206 is a method of treating a disease or disorder, comprising administering the engineered human T cell or pharmaceutical composition of any one of embodiments 192-204 to a subject in need thereof.

Embodiment 207 is the engineered human T cell or pharmaceutical composition of any one of embodiments 192-204, for use in administration to a subject as an adoptive cell transfer (ACT) therapy, for use in treating a subject having a cancer, for use in treating a subject having an infectious disease, or for use in treating a subject having an autoimmune disease.

Embodiment 208 is the method of embodiment 206, wherein the disease or disorder is a cancer.

Embodiment 209 is the engineered human T cell or pharmaceutical composition for use of embodiment 207 or the method of embodiment 208, wherein the cancer is a solid tumor or a hematological malignancy.

Embodiment 210 is the engineered human T cell or pharmaceutical composition for use or the method of embodiment 209, wherein the solid tumor is renal cell carcinoma, or wherein the hematological malignancy is acute myeloid leukemia or multiple myeloma.

### I. Definitions

Unless stated otherwise, the following terms and phrases as used herein are intended to have the following meanings:

The term “or combinations thereof” as used herein refers to all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, CBBA, CABA, and so forth. The skilled artisan will understand that typically there is no limit on the number of items or terms in any combination, unless otherwise apparent from the context.

As used herein, the term “kit” refers to a packaged set of related components, such as one or more polynucleotides or compositions and one or more related materials such as delivery devices (e.g., syringes), solvents, solutions, buffers, instructions, or desiccants.

An “allogeneic” cell, as used herein, refers to a cell originating from a donor subject of the same species as a recipient subject, wherein the donor subject and recipient subject have genetic dissimilarity, e.g., genes at one or more loci that are not identical. Thus, e.g., a cell is allogeneic with respect to the subject to be administered the cell. As used herein, a cell that is removed or isolated from a donor, that will not be re-introduced into the original donor, is considered an allogeneic cell.

The term “allogeneic T-cell target” is used interchangeably herein, and refers to a protein that mediates or contributes to a host versus graft response, mediates or contributes to a graft versus host response; and the gene encoding said molecule and its associated regulatory elements, e.g., promoters. It will be understood that the term allogeneic T-cell target refers to the gene (and its associated regulatory elements) encoding an allogeneic T-cell target protein when it is used in connection with a target sequence or gRNA molecule. Without being bound by theory, inhibition or elimination of one or more allogeneic T-cell targets, e.g., by the methods and compositions disclosed herein, may improve the efficacy, survival, function and/or viability of an allogeneic cell, e.g., an allogeneic T-cell, for example, by reducing or eliminating undesirable immunogenicity (such as a host versus graft response or a graft versus host response).

In a non-limiting example, the protein that mediates or contributes to a graft versus host response or host versus graft response is one or more components of the T-cell receptor. In an embodiment, the component of the T-cell receptor is the T-cell receptor alpha, for example the constant domain of the TCR alpha. In an embodiment, the component of the T-cell receptor is the T-cell receptor beta chain, for example the constant domain 1 or constant domain 2 of the TCR beta. Thus, in embodiments where the protein encoded by the allogeneic T-cell target is a component of the TCR, the gene encoding the allogeneic T-cell target may be, for example, TRAC and combinations thereof.

In a non-limiting example, the protein that mediates or contributes to a graft versus host response or host versus graft response is an HLA protein. Examples of HLA proteins include HLA-A and HLA-B. Thus, in embodiments where the allogeneic T-cell target protein is an HLA protein, the gene encoding the allogeneic T-cell target may be, for example, HLA-A, HLA-B, and a combination thereof.

In a non-limiting example, the protein that mediates or contributes to a graft versus host response or host versus graft response is a major histocompatibility complex class II (MHC II) molecule (e.g., HLA-Dx (where x refers to a letter of a MHC II protein, e.g., HLA-DM, HLA-DO, HLA-DR, HLA-DQ and/or HLA-DP)), or a regulatory factor for expression of a MHC II, and combinations thereof. A non-limiting example is CIITA (also referred to herein as C2TA). Thus, in embodiments where the allogeneic T-cell target protein is a CIITA, the gene encoding the allogeneic T-cell target may be, for example, CIITA. An “autologous” cell, as used herein, refers to a cell derived from the same subject to whom the material will later be re-introduced. Thus, e.g., a cell is considered autologous if it is removed from a subject and it will then be re-introduced into the same subject.

The term “CD70,” as used herein in the context of CD70 protein, refers to the cytokine belonging to the tumor necrosis factor (TNF) family of ligands. “CD70” as used herein in the context of nucleic acids refers to the gene encoding the CD70 protein molecule. The human gene has accession number NC_000019.10 (6581648 . . . 6591150).

“CIITA” or “C2TA,” as used herein, refers to the nucleic acid sequence or protein sequence of “class II major histocompatibility complex transactivator;” the human gene has accession number NC_000016.10 (range 10866208 . . . 10941562), reference GRCh38.p13. The CIITA protein in the nucleus acts as a positive regulator of MHC class II gene transcription and is required for MHC class II protein expression.

As used herein, “MHC” or “MHC molecule(s)” or “MHC protein” or “MHC complex(es),” refers to a major histocompatibility complex molecule (or plural), and includes e.g., MHC class I and MHC class II molecules. In humans, MHC molecules are referred to as “human leukocyte antigen” complexes or “HLA molecules” or “HLA protein.” The use of terms “MHC” and “HLA” are not meant to be limiting; as used herein, the term “MHC” may be used to refer to human MHC molecules, i.e., HLA molecules. Therefore, the terms “MHC” and “HLA” are used interchangeably herein.

The term “HLA-A,” as used herein in the context of HLA-A protein, refers to the MHC class I protein molecule, which is a heterodimer consisting of a heavy chain (encoded by the HLA-A gene) and a light chain (i.e., beta-2 microglobulin). The term “HLA-A” or “HLA-A gene,” as used herein in the context of nucleic acids refers to the gene encoding the heavy chain of the HLA-A protein molecule. The HLA-A gene is also referred to as “HLA class I histocompatibility, A alpha chain;” the human gene has accession number NC_000006.12 (29942532 . . . 29945870). The HLA-A gene is known to have thousands of different versions (also referred to as “alleles”) across the population (and an individual may receive two different alleles of the HLA-A gene). A public database for HLA-A alleles, including sequence information, may be accessed at IPD-IMGT/HLA: https://www.ebi.ac.uk/ipd/imgt/hla/. All alleles of HLA-A are encompassed by the terms “HLA-A” and “HLA-A gene.”

“HLA-B” as used herein in the context of nucleic acids refers to the gene encoding the heavy chain of the HLA-B protein molecule. The HLA-B is also referred to as “HLA class I histocompatibility, B alpha chain;” the human gene has accession number NC_000006.12 (31353875 . . . 31357179).

“HLA-C” as used herein in the context of nucleic acids refers to the gene encoding the heavy chain of the HLA-C protein molecule. The HLA-C is also referred to as “HLA class I histocompatibility, C alpha chain;” the human gene has accession number NC_000006.12 (31268749 . . . 31272092).

The term “TGFβR2” or “TGFBR2” as used herein in the context of protein, refers to a transmembrane protein that has a protein kinase domain, forms a heterodimeric complex with Transforming Growth Factor Beta (TGF-β) receptor type-1, and binds TGF-beta. The term “TGFβR2” or “TGFBR2” as used herein in the context of nucleic acids refers to the gene encoding the Transforming Growth Factor Beta (TGF-β) receptor type-2 protein molecule. The human gene has accession number NC_000003.12 (30606356 . . . 30694142).

The term “TRAC,” as used herein in the context of TRAC protein, refers to the T-cell receptor α-chain. “TRAC” as used herein in the context of nucleic acids refers to the gene encoding the T-cell receptor α-chain. A human wild-type TRAC sequence is available at NCBI Gene ID: 28755; Ensembl: ENSG00000277734. T Cell Receptor Alpha Constant, TCRA, IMD7, TRCA and TRA are gene synonyms for TRAC.

As used herein, the term “within the genomic coordinates” includes the boundaries of the genomic coordinate range given. For example, if chr6: 29942854-chr6: 29942913 is given, the coordinates chr6: 29942854-chr6: 29942913 are encompassed. Throughout this application, the referenced genomic coordinates are based on genomic annotations in the GRCh38 (also referred to as hg38) assembly of the human genome from the Genome Reference Consortium, available at the National Center for Biotechnology Information website. Tools and methods for converting genomic coordinates between one assembly and another are known in the art and can be used to convert the genomic coordinates provided herein to the corresponding coordinates in another assembly of the human genome, including conversion to an earlier assembly generated by the same institution or using the same algorithm (e.g., from GRCh38 to GRCh37), and conversion of an assembly generated by a different institution or algorithm (e.g., from GRCh38 to NCBI33, generated by the International Human Genome Sequencing Consortium). Available methods and tools known in the art include, but are not limited to, NCBI Genome Remapping Service, available at the National Center for Biotechnology Information website, UCSC LiftOver, available at the UCSC Genome Brower website, and Assembly Converter, available at the Ensembl.org website.

As used herein, the term “homozygous” refers to having two identical alleles of a particular gene.

As used herein, the term “subject” is intended to include living organisms in which an immune response can be elicited, including e.g., mammals, primates, humans.

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and 04-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43 (42): 13233-41). RNA and DNA have different sugar moieties and can differ by the presence of uracil or analogs thereof in RNA and thymine or analogs thereof in DNA.

“Guide RNA”, “gRNA”, and simply “guide” are used herein interchangeably to refer to, for example, the guide that directs an RNA-guided DNA binding agent to a target DNA and can be a single guide RNA, or the combination of a crRNA and a trRNA (also known as tracrRNA). Exemplary gRNAs include Class II Cas nuclease guide RNAs, in modified or unmodified forms. The crRNA and trRNA may be associated as a single RNA molecule (single guide RNA, sgRNA) or in two separate RNA strands (dual guide RNA, dgRNA). “Guide RNA” or “gRNA” refers to each type. The trRNA may be a naturally occurring sequence, or a trRNA sequence with modifications or variations compared to naturally-occurring sequences.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 19, 20, 21, 22, 23, or 24, or 25 nucleotides in length, e.g., in the case of Neisseria meningitides. In some embodiments, the Nme Cas9 guide sequence comprises at least 22, 23, or 24 contiguous nucleotides of a sequence selected from SEQ ID NOs: 2-80, 101-120, 201, 265, 301, 302, 304-576, and 601-774. In some embodiments, the target sequence is in a gene or on a chromosome, for example, and is complementary to the guide sequence. In some embodiments, the degree of complementarity or identity between a guide sequence and its corresponding target sequence is at least 80%, 85%, 90%, or 95%. For example, in some embodiments, the guide sequence comprises a sequence 24 contiguous nucleotides of a sequence selected from SEQ ID NO: 2-80, 101-120, 201, 265, 301, 302, 304-576, and 601-774. In some embodiments, the guide sequence and the target region may be 100% complementary or identical. In other embodiments, the guide sequence and the target region may contain at least one mismatch, i.e., one nucleotide that is not identical or not complementary, depending on the reference sequence. For example, the guide sequence and the target sequence may contain 1-2, preferably no more than 1 mismatch, where the total length of the target sequence is 19, 20, 21, 22, 23, or 24, nucleotides, or more. In some embodiments, the guide sequence and the target region may contain 1-2 mismatches where the guide sequence comprises at least 24 nucleotides, or more. In some embodiments, the guide sequence and the target region may contain 1-2 mismatches where the guide sequence comprises 24 nucleotides. That is, the guide sequence and the target region may form a duplex region having base pairs, or more. In certain embodiments, the duplex region may include 1-2 mismatches such that guide strand and target sequence are not fully complementary. Mismatch positions are known in the art as provided in, for example, PAM distal mismatches tend to be better tolerated than PAM proximal matches. Mismatch tolerances at other positions are known in the art (see, e.g., Edraki et al., 2019. Mol. Cell, 73:1-13).

Target sequences for RNA-guided DNA binding agents include both the positive and negative strands of genomic DNA (i.e., the sequence given and the sequence's reverse compliment), as a nucleic acid substrate for an RNA-guided DNA binding agent is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the presence of a PAM and the sequence of the guide RNA. Exemplary RNA-guided DNA binding agents include Cas cleavases/nickases and inactivated forms thereof (“dCas DNA binding agents”). “Cas nuclease”, also called “Cas protein” as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases.

As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants of Spy Cas9 and D16A and H588A of Nme Cas9, e.g., Nme2 Cas9), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9 (1.0) (e.g., K810A, K1003A, R1060A variants), and eSPCas9 (1.1) (e.g., K848A, K1003A, R1060A variants) proteins and modifications thereof. Cpf1 protein, Zetsche et al., Cell, 163:1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13 (11): 722-36 (2015); Shmakov et al., Molecular Cell, 60:385-397 (2015).

Several Cas9 orthologs have been obtained from N. meningitidis (Esvelt et al., NAT. METHODS, vol. 10, 2013, 1116-1121; Hou et al., PNAS, vol. 110, 2013, pages 15644-15649) (NmelCas9, Nme2Cas9, and Nme3Cas9). The Nme2Cas9 ortholog functions efficiently in mammalian cells, recognizes an N4CC PAM, and can be used for in vivo editing with cognate gRNAs (Ran et al., NATURE, vol. 520, 2015, pages 186-191; Kim et al., NAT. COMMUN., vol. 8, 2017, pages 14500). Nme2Cas9 can be specific and selective, e.g., capable of low off-target editing (Lee et al., MOL. THER., vol. 24, 2016, pages 645-654; Kim et al., 2017). See also e.g., WO/2020081568 (e.g., pages 28 and 42), describing an Nme2Cas9 D16A nickase, the contents of which are hereby incorporated by reference in its entirety. Throughout, “NmeCas9” or “Nme Cas9” is generic and encompasses any type of NmeCas9, including, Nmel Cas9, Nme2Cas9, and Nme3Cas9.

Exemplary nucleotide and polypeptide sequences of Cas9 molecules are provided in Table 10. Methods for identifying alternate nucleotide sequences encoding Cas9 polypeptide sequences, including alternate naturally occurring variants, are known in the art. Sequences with at least 75%, 80%, 85%, 90%, 95%, 96%, 97%, 98%, or 99% identity to any of the Cas9 nucleic acid sequences, or nucleic acid sequences encoding the amino acid sequences provided herein are also contemplated.

As used herein, the term “editor” refers to an agent comprising a polypeptide that is capable of making a modification within a DNA sequence. In some embodiments, the editor is a cleavase, such as a Cas9 cleavase. In some embodiments, the editor is capable of deaminating a base within a DNA molecule, and it may be called a base editor. In some embodiments, the editor is capable of deaminating a cytosine (C) in DNA. In some embodiments, the editor is a fusion protein comprising an RNA-guided nickase fused to a cytidine deaminase. In some embodiments, the editor is a fusion protein comprising an RNA-guided nickase fused to an APOBEC3A deaminase (A3A). In some embodiments, the editor comprises a Cas9 nickase fused to an APOBEC3A deaminase (A3A). In some embodiments, the editor is a fusion protein comprising an RNA-guided nickase fused to a cytidine deaminase and a UGI. In some embodiments, the editor lacks a UGI.

As used herein, a “cytidine deaminase” means a polypeptide or complex of polypeptides that is capable of cytidine deaminase activity, that is catalyzing the hydrolytic deamination of cytidine or deoxycytidine, typically resulting in uridine or deoxyuridine. Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, 2005; Conticello, Genome Biol. 9:229, 2008; Muramatsu et al., J. Biol. Chem. 274:18470-6, 1999); Carrington et al., Cells 9:1690 (2020)). In some embodiments, variants of any known cytidine deaminase or APOBEC protein are encompassed. Variants include proteins having a sequence that differs from wild-type protein by one or several mutations (i.e., substitutions, deletions, insertions), such as one or several single point substitutions. For instance, a shortened sequence could be used, e.g., by deleting N-terminal, C-terminal, or internal amino acids, preferably one to four amino acids at the C-terminus of the sequence. As used herein, the term “variant” refers to allelic variants, splicing variants, and natural or artificial mutants, which are homologous to a reference sequence. The variant is “functional” in that it shows a catalytic activity of DNA editing.

As used herein, the term “APOBEC3A” refers to a cytidine deaminase such as the protein expressed by the human A3A gene. The APOBEC3A may have catalytic DNA editing activity. An amino acid sequence of APOBEC3A has been described (UniPROT accession ID: p31941) and is included herein as SEQ ID NO: 850. In some embodiments, the APOBEC3A protein is a human APOBEC3A protein or a wild-type protein. Variants include proteins having a sequence that differs from wild-type APOBEC3A protein by one or several mutations (i.e., substitutions, deletions, insertions), such as one or several single point substitutions. For instance, a shortened APOBEC3A sequence could be used, e.g., by deleting N-terminal, C-terminal, or internal amino acids, preferably one to four amino acids at the C-terminus of the sequence. As used herein, the term “variant” refers to allelic variants, splicing variants, and natural or artificial mutants, which are homologous to an APOBEC3A reference sequence. The variant is “functional” in that it shows a catalytic activity of DNA editing. In some embodiments, an APOBEC3A (such as a human APOBEC3A) has a wild-type amino acid position 57 (as numbered in the wild-type sequence). In some embodiments, an APOBEC3A (such as a human APOBEC3A) has an asparagine at amino acid position 57 (as numbered in the wild-type sequence).

As used herein, a “nickase” is an enzyme that creates a single-strand break (also known as a “nick”) in double strand DNA, i.e., cuts one strand but not the other of the DNA double helix. As used herein, an “RNA-guided DNA nickase” means a polypeptide or complex of polypeptides having DNA nickase activity, wherein the DNA nickase activity is sequence-specific and depends on the sequence of the RNA. Exemplary RNA-guided DNA nickases include Cas nickases. Class 2 Cas nickases include, polypeptides in which either the HNH or RuvC catalytic domain is inactivated, for example, Cas9 (e.g., H840A, D10A, or N863A variants of SpyCas9 or D16A variant of NmeCas9). Exemplary amino acid substitutions in the HNH or HNH-like nuclease domain or RuvC or RuvC-like domains for N. meningitidis include Nme2Cas9 D16A (HNH nickase) and Nme2Cas9 H588A (RuvC nickase). Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9 (1.0) (e.g, K810A, K1003A, R1060A variants), and eSPCas9 (1.1) (e.g., K848A, K1003A, R1060A variants) proteins and modifications thereof. Cpf1 protein, Zetsche et al., Cell, 163:1-13 (2015), is homologous to Cas9, and contains a RuvC-like protein domain. Cpf1 sequences of Zetsche are incorporated by reference in their entirety. See, e.g., Zetsche, Tables S1 and S3. “Cas9” encompasses S. pyogenes (Spy) Cas9, the variants of Cas9 listed herein, and equivalents thereof. See, e.g., Makarova et al., Nat Rev Microbiol, 13 (11): 722-36 (2015); Shmakov et al., Molecular Cell, 60:385-397 (2015).

As used herein, the term “fusion protein” refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “chimeric antigen receptor,” or alternatively “CAR,” refers to a set of polypeptides, typically two in the simplest embodiments, which, when in an immune effector cell, provides the cell with specificity for a target cell, typically a cancer cell, and with intracellular signal generation. In some embodiments, a CAR comprises at least an extracellular antigen binding domain, a transmembrane domain and an intracellular domain (also referred to herein as “an intracellular domain”) comprising a functional signaling domain derived from a stimulatory molecule (e.g., an activation domain) and/or costimulatory molecule (e.g., a costimulatory domain) as defined below. In some aspects, the set of polypeptides are contiguous with each other. In some embodiments, the set of polypeptides includes a dimerization switch that, upon the presence of a dimerization molecule, can couple the polypeptides to one another, e.g., can couple an antigen binding domain to an intracellular domain. In one aspect, the stimulatory molecule is the zeta chain associated with the T-cell receptor complex. In one aspect, the cytoplasmic signaling domain further comprises one or more functional signaling domains derived from at least one costimulatory molecule as defined below. In one aspect, the costimulatory molecule is chosen from the costimulatory molecules described herein, e.g., 41BB (i.e., CD137), CD27 and/or CD28. In one aspect, the CAR comprises a chimeric fusion protein comprising an extracellular antigen binding domain, a transmembrane domain and an intracellular domain comprising a functional signaling domain derived from a stimulatory molecule. In one aspect, the CAR comprises a chimeric fusion protein comprising an extracellular antigen binding domain, a transmembrane domain and an intracellular domain comprising a functional signaling domain derived from a costimulatory molecule and a functional signaling domain derived from a stimulatory molecule. In one aspect, the CAR comprises a chimeric fusion protein comprising an extracellular antigen binding domain, a transmembrane domain and an intracellular domain comprising two functional signaling domains derived from one or more costimulatory molecule(s) and a functional signaling domain derived from a stimulatory molecule. In one aspect, the CAR comprises a chimeric fusion protein comprising an extracellular antigen binding domain, a transmembrane domain and an intracellular domain comprising at least two functional signaling domains derived from one or more costimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen binding domain, wherein the leader sequence is optionally cleaved from the antigen binding domain (e.g., an scFv) during cellular processing and localization of the CAR to the cellular membrane.

A CAR that comprises an antigen binding domain (e.g., an scFv, or TCR) that targets a specific tumor marker X, such as those described herein, is also referred to as an anti-X CAR. For example, a CAR that comprises an antigen binding domain that targets CD19 is referred to as an anti-CD19 CAR. As another example, a CAR that comprises an antigen binding domain that targets BCMA is referred to as an anti-BCMA CAR. The term “signaling domain” refers to the functional portion of a protein which acts by transmitting information within the cell to regulate cellular activity via defined signaling pathways by generating second messengers or functioning as effectors by responding to such messengers.

The term “linker,” as used herein, refers to a chemical group or a molecule linking two adjacent molecules or moieties. Typically, the linker is positioned between, or flanked by, two groups, molecules, or other moieties and connected to each one via a covalent bond. In some embodiments, the linker is an amino acid or a plurality of amino acids (e.g., a peptide or protein) such as a 16-amino acid residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 901), SGSETPGTSESA (SEQ ID NO: 902), or SGSETPGTSESATPEGGSGGS (SEQ ID NO: 903). In some embodiments, the linker is a peptide linker comprising one or more sequences selected from SEQ ID NOs: 901-991.

As used herein, the term “uracil glycosylase inhibitor” or “UGI” refers to a protein that is capable of inhibiting a uracil-DNA glycosylase (UDG) base-excision repair enzyme.

As used herein, “open reading frame” or “ORF” of a gene refers to a sequence consisting of a series of codons that specify the amino acid sequence of the protein that the gene codes for. The ORF begins with a start codon (e.g., ATG in DNA or AUG in RNA) and ends with a stop codon, e.g., TAA, TAG or TGA in DNA or UAA, UAG, or UGA in RNA.

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with the target sequence and the agent binds to the target sequence; in cases where the agent is a cleavase or nickase, binding can be followed by cleaving or nicking.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“Messenger RNA” or “mRNA” is used herein to refer to a polynucleotide and comprises an open reading frame that can be translated into a polypeptide (i.e., can serve as a substrate for translation by a ribosome and amino-acylated tRNAs). mRNA can comprise one or more modifications as provided below.

As used herein, a “genetic modification” is a change at the DNA level, e.g., induced by a CRISPR/Cas9 gRNA and Cas9 system. A genetic modification may comprise an insertion, deletion, or substitution (i.e., base sequence substitution, i.e., mutation), typically within a defined sequence or genomic locus. A genetic modification changes the nucleic acid sequence of the DNA. A genetic modification may be at a single nucleotide position. A genetic modification may be at multiple nucleotides, e.g., 2, 3, 4, 5 or more nucleotides, typically in close proximity to each other, e.g, contiguous nucleotides. A genetic modification can be in a coding sequence, e.g., an exon sequence. A genetic modification can be at a splice site, i.e., sufficiently close to a splice acceptor site or a splice donor site to disrupt splicing. A genetic modification can include insertion of a nucleotide sequence not endogenous to the genomic locus, e.g., insertion of a coding sequence of a heterologous open reading frame or gene. As used herein, a genetic modification can be used to prevent translation of an endogenous full-length protein having an amino acid sequence of the full-length protein prior to genetic modification of the genomic locus. Prevention of translation of an endogenous full-length protein or gene product includes prevention of translation of a protein or gene product of any length. Translation of an endogenous full-length protein can be prevented, for example, by a frameshift mutation that results in the generation of a premature stop codon or by generation of a nonsense mutation. Translation of an endogenous full-length protein can be prevented by disruption of splicing. Translation of an endogenous full-length protein can be prevented by the insertion of a heterologous coding sequence. Translation of an endogenous full-length protein, e.g., when the endogenous full-length protein contains an unwanted mutation, can be prevented by making a change at one or more positions to change an endogenous full-length protein coding sequence to provide a modified full-length coding sequence different from the endogenous sequence present in the cell, e.g., correction of a point mutation. Translation of an endogenous full-length protein can be prevented by altering the splicing of the endogenous full-length protein to produce a different protein by alternative splicing.

As used herein, “indel” refers to an insertion or deletion mutation consisting of a number of nucleotides that are either inserted, deleted, or inserted and deleted, e.g., at the site of double-stranded breaks (DSBs), in a target nucleic acid. As used herein, when indel formation results in an insertion, the insertion is a random insertion at the site of a DSB and may or may not be directed by or based on a template sequence.

As used herein, a “heterologous coding sequence” refers to a coding sequence that has been introduced as an exogenous source within a cell (e.g., inserted at a genomic locus such as a safe harbor locus including a TCR gene locus). That is, the introduced coding sequence is heterologous with respect to at least its insertion site. A polypeptide expressed from such heterologous coding sequence gene is referred to as a “heterologous polypeptide.” The heterologous coding sequence can be naturally occurring or engineered, and can be wild-type or a variant. The heterologous coding sequence may include nucleotide sequences other than the sequence that encodes the heterologous polypeptide (e.g., an internal ribosomal entry site). The heterologous coding sequence can be a coding sequence that occurs naturally in the genome, as a wild-type or a variant (e.g., mutant). For example, although the cell contains the coding sequence of interest (as a wild-type or as a variant), the same coding sequence or variant thereof can be introduced as an exogenous source for, e.g., expression at a locus that is highly expressed. The heterologous coding sequence can also be a coding sequence that is not naturally occurring in the genome, or that expresses a heterologous polypeptide that does not naturally occur in the genome. “Heterologous coding sequence”, “exogenous coding sequence”, and “transgene” are used interchangeably. In some embodiments, the heterologous coding sequence or transgene includes an exogenous nucleic acid sequence, e.g., a nucleic acid sequence is not endogenous to the recipient cell. In some embodiments, the heterologous coding sequence or transgene includes an exogenous nucleic acid sequence, e.g., a nucleic acid sequence that does not naturally occur in the recipient cell. For example, a heterologous coding sequence may be heterologous with respect to its insertion site and with respect to its recipient cell.

As used herein, “reduced or eliminated” expression of a protein on a cell refers to a partial or complete loss of expression of the protein relative to an unmodified cell. In some embodiments, the surface expression of a protein on a cell is measured by flow cytometry and has “reduced” or “eliminated” surface expression relative to an unmodified cell as evidenced by a reduction in fluorescence signal upon staining with the same antibody against the protein. A cell that has “reduced” or “eliminated” surface expression of a protein by flow cytometry relative to an unmodified cell may be referred to as “negative” for expression of that protein as evidenced by a fluorescence signal similar to a cell stained with an isotype control antibody. The “reduction” or “elimination” of protein expression can be measured by other known techniques in the field with appropriate controls known to those skilled in the art.

As used herein, “knockdown” refers to a decrease in expression of a particular gene target or protein encoded by a gene target (e.g., protein, mRNA, or both), e.g., as compared to expression of an unedited target sequence. Knockdown of a protein can be measured by detecting total cellular amount of the protein from a sample, such as a tissue, fluid, or cell population of interest. It can also be measured by measuring a surrogate, marker, or activity for the protein. Methods for measuring knockdown of mRNA are known and include analyzing mRNA isolated from a sample of interest. In some embodiments, “knockdown” may refer to some loss of expression of a particular gene target, for example a decrease in the amount of mRNA transcribed or a decrease in the amount of protein expressed by a cell or population of cells (including in vivo populations such as those found in tissues).

As used herein, “knockout” refers to a loss of expression from a particular gene or of a particular protein in a cell. Knockout can result in a decrease in expression below the level of detection of the assay. Knockout can be measured either by detecting total cellular amount of a protein in a cell, a tissue or a population of cells.

As used herein, a “target sequence” or “genomic target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

As used herein, “treatment” refers to any administration or application of a therapeutic for disease or disorder in a subject, and includes inhibiting the disease, arresting its development, relieving one or more symptoms of the disease, curing the disease, or preventing one or more symptoms of the disease, including recurrence of the symptom.

Reference will now be made in detail to certain embodiments of the present disclosure, examples of which are illustrated in the accompanying drawings. While the present disclosure is described in conjunction with the illustrated embodiments, it will be understood that they are not intended to limit the present disclosure to those embodiments. On the contrary, the present disclosure is intended to cover all alternatives, modifications, and equivalents, which may be included within the present disclosure as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### II. Anti-CD70 Chimeric Antigen Receptors (CARs)

The present disclosure describes new CARs comprising an anti-CD70 antibody fused to a CAR scaffold and generally with different component transmembrane domains and intracellular domains. The resulting CARs provide for enhanced cytotoxicity against tumor cells, both in vitro and in vivo.

In general, aspects of the present disclosure pertain to or include an isolated nucleic acid molecule encoding an anti-CD70 CAR, wherein the anti-CD70 CAR comprises an antigen binding domain (e.g., antibody or antibody fragment) that binds to a tumor antigen as described herein, i.e., CD70, a transmembrane domain (e.g., a transmembrane domain described herein), and an intracellular domain (e.g., an intracellular domain described herein) (e.g., an intracellular domain comprising a costimulatory domain (e.g., a costimulatory domain described herein) and/or a primary signaling domain (e.g., an activation domain described herein). In other aspects, the present disclosure includes: host cells containing the above nucleic acids and isolated proteins encoded by such nucleic acid molecules. CAR nucleic acid constructs, encoded proteins, containing vectors, host cells, pharmaceutical compositions, and methods of administration and treatment related to the present disclosure are disclosed in detail in International Patent Application Publication No. WO2015142675, which is incorporated by reference in its entirety.

In one aspect, the present disclosure pertains to an isolated nucleic acid molecule encoding an anti-CD70 CAR, wherein the anti-CD70 CAR comprises an antigen binding domain (e.g., antibody or antibody fragment) that binds to a tumor-supporting antigen (e.g., a tumor-supporting antigen, e.g., CD70, as described herein), a transmembrane domain (e.g., a transmembrane domain described herein), and an intracellular domain (e.g., an intracellular domain described herein) (e.g., an intracellular domain comprising a costimulatory domain (e.g., a costimulatory domain described herein) and/or a primary signaling domain (e.g., an activation domain described herein). In other aspects, the present disclosure features polypeptides encoded by such nucleic acids and host cells containing such nucleic acids and/or polypeptides.

The present disclosure provides cells, e.g., immune effector cells (e.g., T-cells, NK cells), comprising a gRNA molecule or CRISPR system as described herein, or comprising a genetic modification within genomic coordinates targeted by a guide RNA or CRISPR system as described herein, that are further engineered to contain one or more anti-CD70 CARs that direct the immune effector cells to undesired cells (e.g., cancer cells). This is achieved through an antigen binding domain on the anti-CD70 CAR that is specific for a cancer associated antigen, e.g., CD70.

The disclosed anti-CD70 CARs further comprise a transmembrane domain, or a functional fragment thereof. In some embodiments, the transmembrane domain is a CD8a or CD28 transmembrane domain. The disclosed anti-CD70 CARs further comprise a hinge domain, or a functional fragment thereof, between the antigen binding protein and the transmembrane domain.

The disclosed anti-CD70 CARs may further comprise an intracellular domain comprising one or more of an activation domain and/or a costimulatory signaling domain (or a costimulatory domain). In some embodiments, the intracellular domain comprises a sequence encoding an activation domain. In some embodiments, the intracellular domain comprises a costimulatory signaling domain. In some embodiments, the intracellular domain comprises an activation domain and a costimulatory signaling domain. In some embodiments, the intracellular domain comprises a costimulatory domain, or a functional fragment thereof, the costimulatory domain is a 4-1BB or CD28 costimulatory domain.

The present disclosure also encompasses isolated nucleic acid molecules comprising sequences encoding the disclosed amino acid sequences and CARs. It should be noted that where an amino acid sequence is described, the nucleic acid sequence that encodes the amino acid sequence is also included.

The disclosed anti-CD70 CARs further comprise a transmembrane domain, or a functional fragment thereof. In some embodiments, the transmembrane domain is a CD8a or CD28 transmembrane domain.

In some embodiments, the present disclosure provides for an engineered cell comprising the nucleic acid, the mRNA, or the expression vector. In some embodiments, the present disclosure provides for an engineered cell comprising the anti-CD70 CAR. In some embodiments, the present disclosure provides for an engineered cell comprising the anti-CD70 CAR, wherein the cell is transduced with an expression vector operably linked to or comprising a nucleic acid encoding the anti-CD70 CAR, and wherein the expression vector directs expression of the anti-CD70 CAR in the cell. In some embodiments, the present disclosure provides for the expression vector or the engineered cell, wherein the expression vector comprises a retroviral or lentiviral expression vector.

Exemplary anti-CD70 CAR polypeptide and nucleic acid sequences are shown in Tables 1A and 1B below.

TABLE 1A
Exemplary Anti-CD70 CARs

| | Antigen | | Trans- | | |
|---|---|---|---|---|---|
| | Binding | Hinge | membrane | Costimulatory | Activation |
| Construct | Protein | Domain | Domain | Domain | Domain |
| 5280 | 10B4 | CD8a | CD28 | CD28 | CD3z |
| 5716 | 10B4 | CD8a | CD8a | 41BB | CD3z |
| 5284 | 18E7 | CD8a | CD28 | CD28 | CD3z |
| 5718 | 18E7 | CD8a | CD8a | 41BB | CD3z |
| 5281 | 1F4 | CD8a | CD28 | CD28 | CD3z |
| 5719 | 1F4 | CD8a | CD8a | 41BB | CD3z |
| 5714 | 7F2 | CD8a | CD8a | 41BB | CD3z |
| 6115 | 8A1 | CD8a | CD8a | CD28 | CD3z |
| 5715 | 8A1 | CD8a | CD8a | 41BB | CD3z |
| 5283 | 8B5 | CD8a | CD28 | CD28 | CD3z |
| 5717 | 8B5 | CD8a | CD8a | 41BB | CD3z |
| 4645 | | CD8a | CD8a | 41BB | CD3z |
| (Benchmark) | | | | | |

TABLE 1B
Exemplary Anti-CD70 CARs

| SEQ ID | Construct | |
|---|---|---|
| NO. | Name | Sequence |
| 1 | Construct | ATGGCCCTGCCTGTCACCGCGTTGCTTCTTCCCCTGGCGCTGCTGCTCCACGCCGC |
| | 5281 CAR | TCGCCCCGAGGTGCAGCTGCTAGAAAGCGGGGGGGGGCTGGTGCAGCCAGGCGGGA |
| | ORF | GCCTCCGTCTCTCCTGCGCTGCTTCCGGCTTTACCTTTTCGATTTACGCCATGAGT |
| | | TGGGTGCGCCAGGCCCCGGGTAAGGGCCTGGAGTGGGTCTCGGCTATCAGCGATTC |
| | | TGGCGGTCGCACGTACTTCGCGGACAGCGTGCGTGGCCGGTTCACCATCTCCCGCG |
| | | ACAATTCTAAGAACACGTTGTCCCTGCAGATGAACTCCCTGAGAGCCGAGGACACT |
| | | GCCGTGTACTACTGTGCCAAGGTGGATTATAGCAACTACCTCTTTTTCGACTATTG |
| | | GGGCCAGGGCACCCTGGTGACCGTGTCGAGCGGAGGTGGAGGTTCCGGCGGGGGAG |
| | | GCTCCGGAGGCGGCGGATCTGAGATCGTGCTGACCCAGTCTCCGGGCACCCTGTCT |
| | | CTGTCTCCTGGAGAGCGCGCCACCCTGAGTTGCCGAGCTTCTCAGAGCATCAGCTC |
| | | TAGCTACCTGGCCTGGTACCAGCAGAAGCCGGGACAGGCCCCTCGCCTGCTCATCT |
| | | ACGGCGCTTCCTCGAGAGCCACTGGCATCCCTGACCGCTTCTCTGGATCGGGCTCC |
| | | GGAACAGATTTCACTCTGACTATTTCTCGGTTGGAGCCCGAAGACTTCGCTGTCTA |
| | | CTATTGTCAACAGTATGGATCCTCCCCGTACACCTTTGGTCAGGGCACCAAGCTGG |
| | | AGATCAAAGGGTCCACTACGACGCCCGCCCCGAGGCCCCCTACCCCCGCACCAACC |
| | | ATTGCGTCCCAGCCGTTGAGCCTGCGGCCTGAAGCTTGCCGACCGGCAGCGGGTGG |
| | | CGCCGTCCACACTCGCGGTTTGGATTTCGCTTGTGACAAAGACCCCAAGTTCTGGG |
| | | TACTTGTTGTGGTGGGGGGCGTATTAGCATGCTACTCCCTTCTGGTTACCGTCGCG |
| | | TTCATCATCTTCTGGGTCAGGTCGAAGCGCAGCCGCCTGCTCCATAGCGACTACAT |
| | | GAATATGACCCCCCGTCGGCCTGGCCCCACACGCAAGCACTACCAGCCCTACGCCC |
| | | CCCCAAGAGACTTCGCGGCCTACCGCTCACGCGTGAAGTTTTCACGCTCTGCGGAC |
| | | GCTCCCGCTTATCAGCAGGGCCAGAACCAGCTTTACAACGAGCTTAACCTGGGCCG |
| | | CCGAGAGGAGTACGATGTGCTGGACAAGCGCAGGGGCCGTGACCCGGAGATGGGCG |
| | | GGAAGCCTCAGCGCCGCAAAAACCCACAGGAGGGCCTGTACAACGAGCTGCAGAAG |
| | | GACAAAATGGCCGAGGCCTACTCCGAGATAGGTATGAAGGGCGAGCGCCGGCGTGG |
| | | TAAAGGCCACGATGGCCTCTATCAGGGTCTGTCCACCGCCACCAAGGACACCTACG |
| | | ACGCACTGCATATGCAAGCGTTACCACCCCGCTAA |
| 2 | Construct | MALPVTALLLPLALLLHAARPEVQLLESGGGLVQPGGSLRLSCAASGFTFSIYAMS |
| | 5281 CAR | WVRQAPGKGLEWVSAISDSGGRTYFADSVRGRFTISRDNSKNTLSLQMNSLRAEDT |
| | AA | AVYYCAKVDYSNYLFFDYWGQGTLVTVSSGGGGSGGGGSGGGGSEIVLTQSPGTLS |
| | sequence | LSPGERATLSCRASQSISSSYLAWYQQKPGQAPRLLIYGASSRATGIPDRFSGSGS |
| | | GTDFTLTISRLEPEDFAVYYCQQYGSSPYTFGQGTKLEIKGSTTTPAPRPPTPAPT |
| | | IASQPLSLRPEACRPAAGGAVHTRGLDFACDKDPKFWVLVVVGGVLACYSLLVTVA |
| | | FIIFWVRSKRSRLLHSDYMNMTPRRPGPTRKHYQPYAPPRDFAAYRSRVKFSRSAD |
| | | APAYQQGQNQLYNELNLGRREEYDVLDKRRGRDPEMGGKPQRRKNPQEGLYNELQK |
| | | DKMAEAYSEIGMKGERRRGKGHDGLYQGLSTATKDTYDALHMQALPPR* |
| 3 | Construct | atggccctgcctgtgacagctctgctcctccctctggccctgctgctccatgccgc |
| | 5719 CAR | cagacccGAGGTGCAGCTCCTGGAGAGTGGTGGAGGCCTGGTCCAGCCAGGGGGCT |
| | ORF | CCTTGCGTCTGTCTTGCGCCGCTAGCGGCTTCACTTTTTCTATCTACGCCATGAGT |
| | | TGGGTGCGCCAGGCTCCTGGTAAAGGCCTCGAGTGGGTCTCTGCCATCAGCGATTC |
| | | TGGTGGACGCACCTACTTCGCGGACAGCGTACGTGGCCGCTTCACCATCAGCCGGG |
| | | ACAACTCCAAGAACACGCTTAGCCTGCAGATGAATTCCCTCCGCGCGGAGGACACC |
| | | GCAGTCTATTACTGTGCCAAGGTGGATTACAGCAACTACCTGTTCTTCGACTATTG |
| | | GGGCCAGGGAACATTGGTGACCGTTTCCTCTGGTGGCGGGGGTTCCGGCGGCGGCG |
| | | GCTCGGGGGGTGGGGGGTCCGAGATCGTGCTGACCCAGAGTCCTGGCACTCTTTCG |
| | | CTGTCGCCGGGGGAGCGCGCCACTCTGTCATGCCGAGCTTCTCAGTCGATCTCGTC |
| | | CTCTTACCTGGCCTGGTACCAGCAGAAGCCCGGCCAGGCGCCGAGGCTGTTGATTT |
| | | ACGGCGCATCCTCCCGCGCTACCGGCATCCCCGACAGATTTTCAGGCAGCGGCTCC |
| | | GGAACCGACTTCACACTAACCATTTCTCGCCTGGAACCCGAGGACTTTGCCGTGTA |
| | | CTACTGTCAACAGTACGGTTCCAGCCCCTATACCTTCGGCCAGGGCACCAAGCTGG |
| | | AGATCAAGGGCTCCaccaccacccctgcccctagacctcccaccccagccccaaca |
| | | atcgccagccagcctctgtctctgcggcccgaagcctgtagacctgctgccggcgg |
| | | agccgtgcacaccagaggcctggacttcgcctgcgacatctacatctgggcccctc |
| | | tggccggcacctgtggcgtgctgctgctgagcctggtgatcaccctgtactgcaaa |
| | | cggggcagaaagaaactcctgtatatattcaaacaaccatttatgagaccagtaca |
| | | aactactcaagaggaagatggctgtagctgccgatttccagaagaagaagaaggag |
| | | gatgtgaactgagagtgaagttcagcagatccgccgacgcccctgcctaccagcag |
| | | ggacagaaccagctgtacaacgagctgaacctgggcagacgggaagagtacgacgt |
| | | gctggacaagcggagaggccgggaccccgagatgggcggaaagcccagacggaaga |
| | | acccccaggaaggcctgtataacgaactgcagaaagacaagatggccgaggcctac |
| | | agcgagatcggcatgaagggcgagcggaggcgcggcaagggccacgatggcctgta |
| | | ccagggcctgagcaccgccaccaaggacacctacgacgccctgcacatgcaggccc |
| | | tgccccccagaTAA |
| 4 | Construct | MALPVTALLLPLALLLHAARPEVQLLESGGGLVQPGGSLRLSCAASGFTFSIYAMS |
| | 5719 CAR | WVRQAPGKGLEWVSAISDSGGRTYFADSVRGRFTISRDNSKNTLSLQMNSLRAEDT |
| | AA | AVYYCAKVDYSNYLFFDYWGQGTLVTVSSGGGGSGGGGSGGGGSEIVLTQSPGTLS |
| | sequence | LSPGERATLSCRASQSISSSYLAWYQQKPGQAPRLLIYGASSRATGIPDRFSGSGS |
| | | GTDFTLTISRLEPEDFAVYYCQQYGSSPYTFGQGTKLEIKGSTTTPAPRPPTPAPT |
| | | IASQPLSLRPEACRPAAGGAVHTRGLDFACDIYIWAPLAGTCGVLLLSLVITLYCK |
| | | RGRKKLLYIFKQPFMRPVQTTQEEDGCSCRFPEEEEGGCELRVKFSRSADAPAYQQ |
| | | GQNQLYNELNLGRREEYDVLDKRRGRDPEMGGKPRRKNPQEGLYNELQKDKMAEAY |
| | | SEIGMKGERRRGKGHDGLYQGLSTATKDTYDALHMQALPPR* |
| 5 | Construct | ATGGCCCTGCCTGTCACTGCGCTGCTGCTGCCGCTCGCTCTGCTGCTTCACGCCGC |
| | 6115 CAR | CCGACCGCAGGTGCAGTTGGTGCAGTCTGGCGCCGAGGTGAAGAAGCCGGGGTCGT |
| | ORF | CCGTGAAAGTAAGCTGTAAGGCATCCGGCTACTCCTTCAGCGATTATTACATGAAT |
| | | TGGGTGCGCCAGGCTCCTGGCCAGGGGCTGGAGTGGATGGGAGAGATTAATCCAAC |
| | | CACAGGCGGCGCTACCTATAACCAGAAGTTTAAGGCTCGGGTCACTATTACTGCTG |
| | | ACGAGAGCACCTCCACGGCCTACATGGAACTGTCCTCGCTGCGCTCCGAGGACACC |
| | | GCGGTGTACTACTGTGCTCGTTCCCTGTTCCCCGCCAACTACGCTGGTTTCGTCTA |
| | | TTGGGGCCAGGGCACCCTGGTCACCGTGTCGAGTGGCGGAGGCGGCAGTGGCGGCG |
| | | GCGGCTCGGGAGGCGGTGGCAGCGACATTCAGATGACCCAGAGCCCCTCTTCACTG |
| | | TCCGCCTCCGTTGGGGACCGCGTCACCATCACCTGCTCTGCGTCCTCCTCAGTGAG |
| | | CTACATGTATTGGTACCAGCAAAAGCCCGGCAAGGCCCCCAAGTTGTTGATCTACG |
| | | ACACCAGCAAGCTGGCTAGCGGAGTACCTAGCCGCTTCTCTGGTTCCGGAAGCGGG |
| | | ACCGACTACACGTTCACCATCTCCTCTCTCCAGCCGGAAGACATCGCCACATACTA |
| | | CTGCCAGCAGTGGTCTAGTTATCCGTGGACCTTCGGTCAGGGTACTAAGGTGGAGA |
| | | TCAAGGGCTCCACTACGACCCCCGCCCCTAGGCCCCCAACCCCCGCTCCCACGATC |
| | | GCCTCGCAGCCACTTTCTCTCCGCCCAGAGGCCTGCCGACCTGCCGCGGGCGGTGC |
| | | AGTGCACACACGGGGACTAGACTTTGCTTGTGATAAGGACCCCAAATTTTGGGTCT |
| | | TGGTGGTGGTGGGTGGGGTGCTGGCCTGCTACAGCCTGCTGGTGACCGTGGCGTTC |
| | | ATCATCTTCTGGGTCCGTTCCAAGCGCAGCCGCCTGCTGCACTCTGACTACATGAA |
| | | CATGACTCCGCGGCGCCCCGGCCCTACCCGCAAGCACTACCAGCCGTACGCGCCCC |
| | | CCCGCGATTTCGCCGCCTACCGGTCGCGCGTTAAATTTTCTCGGTCTGCGGACGCC |
| | | CCCGCCTACCAACAGGGGCAGAACCAGCTGTACAACGAGCTCAACCTGGGCAGGAG |
| | | GGAGGAGTACGATGTGCTGGACAAGCGCAGGGGCCGCGATCCAGAGATGGGCGGTA |
| | | AGCCTCAGAGACGTAAAAACCCTCAGGAGGGCCTTTACAACGAACTCCAGAAGGAC |
| | | AAAATGGCGGAGGCATATTCGGAGATCGGTATGAAAGGGGAGCGCCGCCGCGGAAA |
| | | GGGCCATGACGGCCTTTACCAAGGGTTATCCACTGCCACCAAGGATACCTACGACG |
| | | CTCTGCATATGCAGGCGCTGCCCCCGCGT |
| 6 | Construct | MALPVTALLLPLALLLHAARPQVQLVQSGAEVKKPGSSVKVSCKASGYSFSDYYMN |
| | 6115 CAR | WVRQAPGQGLEWMGEINPTTGGATYNQKFKARVTITADESTSTAYMELSSLRSEDT |
| | AA | AVYYCARSLFPANYAGFVYWGQGTLVTVSSGGGGSGGGGSGGGGSDIQMTQSPSSL |
| | sequence | SASVGDRVTITCSASSSVSYMYWYQQKPGKAPKLLIYDTSKLASGVPSRFSGSGSG |
| | | TDYTFTISSLQPEDIATYYCQQWSSYPWTFGQGTKVEIKGSTTTPAPRPPTPAPTI |
| | | ASQPLSLRPEACRPAAGGAVHTRGLDFACDKDPKFWVLVVVGGVLACYSLLVTVAF |
| | | IIFWVRSKRSRLLHSDYMNMTPRRPGPTRKHYQPYAPPRDFAAYRSRVKFSRSADA |
| | | PAYQQGQNQLYNELNLGRREEYDVLDKRRGRDPEMGGKPQRRKNPQEGLYNELQKD |
| | | KMAEAYSEIGMKGERRRGKGHDGLYQGLSTATKDTYDALHMQALPPR |
| 7 | Construct | atggccctgcctgtgacagctctgctcctccctctggccctgctgctccatgccgc |
| | 5715 CAR | cagacccCAGGTGCAGCTGGTGCAGAGCGGCGCCGAGGTGAAGAAGCCCGGCTCCT |
| | ORF | CTGTGAAAGTGTCATGCAAGGCATCTGGCTACTCCTTTTCAGATTATTACATGAAC |
| | | TGGGTCCGCCAGGCCCCTGGTCAGGGACTGGAGTGGATGGGCGAGATTAATCCAAC |
| | | CACCGGCGGAGCAACTTACAACCAGAAATTTAAAGCTCGGGTCACCATCACTGCGG |
| | | ACGAGAGCACAAGCACAGCCTACATGGAGCTGAGCAGCTTGCGCTCGGAGGACACG |
| | | GCCGTGTACTACTGTGCTCGTAGCCTGTTCCCCGCCAACTACGCGGGTTTCGTCTA |
| | | TTGGGGCCAGGGCACTCTCGTAACCGTGTCCTCCGGAGGGGGTGGCTCCGGAGGCG |
| | | GGGGGAGTGGTGGTGGCGGCTCCGACATCCAGATGACCCAGAGCCCATCTTCCCTT |
| | | TCGGCTTCCGTGGGGGACAGGGTGACCATCACCTGCTCTGCTTCGAGTTCCGTTAG |
| | | CTACATGTATTGGTACCAGCAGAAGCCTGGCAAGGCGCCCAAGCTACTCATCTACG |
| | | ACACCAGCAAGCTGGCGTCCGGTGTCCCCTCTCGCTTCTCGGGCTCCGGCTCTGGG |
| | | ACTGACTACACGTTCACCATTTCTTCCCTGCAGCCGGAAGATATCGCCACCTACTA |
| | | CTGTCAACAGTGGTCTTCCTATCCGTGGACCTTCGGACAGGGCACCAAGGTGGAGA |
| | | TCAAGGGCTCGaccaccacccctgcccctagacctcccaccccagccccaacaatc |
| | | gccagccagcctctgtctctgcggcccgaagcctgtagacctgctgccggcggagc |
| | | cgtgcacaccagaggcctggacttcgcctgcgacatctacatctgggcccctctgg |
| | | ccggcacctgtggcgtgctgctgctgagcctggtgatcaccctgtactgcaaacgg |
| | | ggcagaaagaaactcctgtatatattcaaacaaccatttatgagaccagtacaaac |
| | | tactcaagaggaagatggctgtagctgccgatttccagaagaagaagaaggaggat |
| | | gtgaactgagagtgaagttcagcagatccgccgacgcccctgcctaccagcaggga |
| | | cagaaccagctgtacaacgagctgaacctgggcagacgggaagagtacgacgtgct |
| | | ggacaagcggagaggccgggaccccgagatgggcggaaagcccagacggaagaacc |
| | | cccaggaaggcctgtataacgaactgcagaaagacaagatggccgaggcctacagc |
| | | gagatcggcatgaagggcgagcggaggcgcggcaagggccacgatggcctgtacca |
| | | gggcctgagcaccgccaccaaggacacctacgacgccctgcacatgcaggccctgc |
| | | cccccagaTAA |
| 8 | Construct | MALPVTALLLPLALLLHAARPQVQLVQSGAEVKKPGSSVKVSCKASGYSFSDYYMN |
| | 5715 CAR | WVRQAPGQGLEWMGEINPTTGGATYNQKFKARVTITADESTSTAYMELSSLRSEDT |
| | AA | AVYYCARSLFPANYAGFVYWGQGTLVTVSSGGGGSGGGGSGGGGSDIQMTQSPSSL |
| | sequence | SASVGDRVTITCSASSSVSYMYWYQQKPGKAPKLLIYDTSKLASGVPSRFSGSGSG |
| | | TDYTFTISSLQPEDIATYYCQQWSSYPWTFGQGTKVEIKGSTTTPAPRPPTPAPTI |
| | | ASQPLSLRPEACRPAAGGAVHTRGLDFACDIYIWAPLAGTCGVLLLSLVITLYCKR |
| | | GRKKLLYIFKQPFMRPVQTTQEEDGCSCRFPEEEEGGCELRVKFSRSADAPAYQQG |
| | | QNQLYNELNLGRREEYDVLDKRRGRDPEMGGKPRRKNPQEGLYNELQKDKMAEAYS |
| | | EIGMKGERRRGKGHDGLYQGLSTATKDTYDALHMQALPPR* |
| 9 | Construct | ATGGCGCTGCCCGTGACTGCCCTACTCCTGCCCCTTGCTCTGCTTTTGCACGCCGC |
| | 5280 CAR | CCGCCCTCAAATTCAGCTGGTGGAGTCCGGAGGGGGCGTGGTGCAGCCGGGACGCA |
| | ORF | GTCTCCGCCTGTCCTGTGCTGCGAGCGGCTTCACCTTCGGCTACTACGCCATGCAT |
| | | TGGGTGCGCCAGGCCCCCGGCAAAGGCCTGGAGTGGGTCGCCGTGATCAGCTACGA |
| | | TGGCTCGATCAAGTACTACGCGGACTCCGTGAAGGGCCGCTTCACCATTTCTAGGG |
| | | ACAACTCTAAGAACACGCTCTACCTACAGATGAATTCCCTGCGCGCAGAAGACACT |
| | | GCGGTGTACTACTGTGCTCGCGAGGGCCCTTATAGCAACTACCTGGATTATTGGGG |
| | | CCAGGGGACCCTGGTGACCGTGAGCTCTGGGGGGGGGGGCTCCGGTGGAGGCGGTT |
| | | CGGGTGGCGGAGGCTCTGCTATCCAGCTGACCCAGAGCCCGTCTAGCCTTTCTGCG |
| | | TCGGTTGGTGATAGAGTCACCATCACCTGCCGAGCGTCGCAGGGCATCTCATCCGC |
| | | CCTGGCGTGGTACCAGCAGAAGCCGGGAAAGGCTCCCAAGTTCCTGATCTACGACG |
| | | CCTCGTCCCTGGAGAGCGGTGTCCCCTCGCGCTTCTCTGGTAGCGGTTCCGGGACG |
| | | GACTTCACACTGACCATTTCGTCTTTGCAGCCAGAGGACTTTGCCACTTACTATTG |
| | | CCAGCAGTTCAACAGTTATCCCTTCACCTTCGGCCCCGGCACTAAGGTGGACATCA |
| | | AGGGATCCACCACAACTCCCGCGCCGCGTCCCCCGACCCCTGCCCCCACCATCGCC |
| | | TCCCAGCCCCTGAGCCTGCGCCCCGAAGCATGTCGGCCAGCTGCCGGCGGCGCCGT |
| | | GCACACACGCGGATTGGATTTCGCGTGCGACAAAGACCCTAAATTTTGGGTCCTGG |
| | | TTGTAGTGGGCGGTGTGCTGGCCTGCTACTCCCTATTGGTCACCGTGGCGTTCATC |
| | | ATATTCTGGGTCAGGTCCAAGCGCAGCCGTCTGCTGCACAGTGACTACATGAACAT |
| | | GACTCCGCGCCGGCCCGGGCCAACCCGCAAGCACTACCAGCCATACGCACCTCCTC |
| | | GTGACTTTGCCGCTTACCGGTCCAGGGTAAAATTTTCACGCTCCGCTGACGCTCCG |
| | | GCCTATCAGCAGGGTCAGAACCAGCTGTACAACGAGCTCAACCTGGGCCGCCGTGA |
| | | GGAGTACGACGTGTTGGACAAGCGACGCGGCAGAGACCCGGAGATGGGCGGCAAGC |
| | | CACAGCGCCGGAAGAATCCTCAGGAGGGCCTGTACAACGAGCTGCAAAAGGATAAG |
| | | ATGGCTGAAGCTTACTCCGAGATCGGCATGAAAGGGGAGCGCCGCCGTGGTAAGGG |
| | | CCATGATGGCCTTTATCAGGGCCTCAGCACCGCCACCAAGGACACCTACGACGCAT |
| | | TACACATGCAGGCTCTGCCTCCTCGAtga |
| 10 | Construct | MALPVTALLLPLALLLHAARPQIQLVESGGGVVQPGRSLRLSCAASGFTFGYYAMH |
| | 5280 CAR | WVRQAPGKGLEWVAVISYDGSIKYYADSVKGRFTISRDNSKNTLYLQMNSLRAEDT |
| | AA | AVYYCAREGPYSNYLDYWGQGTLVTVSSGGGGSGGGGSGGGGSAIQLTQSPSSLSA |
| | sequence | SVGDRVTITCRASQGISSALAWYQQKPGKAPKFLIYDASSLESGVPSRFSGSGSGT |
| | | DFTLTISSLQPEDFATYYCQQFNSYPFTFGPGTKVDIKGSTTTPAPRPPTPAPTIA |
| | | SQPLSLRPEACRPAAGGAVHTRGLDFACDKDPKFWVLVVVGGVLACYSLLVTVAFI |
| | | IFWVRSKRSRLLHSDYMNMTPRRPGPTRKHYQPYAPPRDFAAYRSRVKFSRSADAP |
| | | AYQQGQNQLYNELNLGRREEYDVLDKRRGRDPEMGGKPQRRKNPQEGLYNELQKDK |
| | | MAEAYSEIGMKGERRRGKGHDGLYQGLSTATKDTYDALHMQALPPR* |
| 11 | Construct | atggccctgcctgtgacagctctgctcctccctctggccctgctgctccatgccgc |
| | 5716 CAR | cagacccCAAATTCAGCTGGTGGAGAGTGGCGGGGGCGTGGTCCAGCCGGGACGCA |
| | ORF | GCTTGCGTCTGTCATGCGCCGCGTCTGGCTTCACTTTCGGCTACTACGCCATGCAC |
| | | TGGGTGCGCCAGGCCCCCGGCAAGGGTCTTGAGTGGGTCGCCGTAATTAGCTACGA |
| | | CGGCTCCATCAAGTACTACGCGGACTCCGTCAAGGGGCGCTTCACCATATCCCGGG |
| | | ACAACTCCAAGAACACGCTGTACTTGCAGATGAACTCCCTGCGCGCCGAAGATACT |
| | | GCCGTGTACTACTGTGCTCGTGAGGGCCCCTATTCGAATTACCTGGACTATTGGGG |
| | | CCAGGGGACCCTGGTTACCGTGTCGTCCGGGGGTGGGGGCTCCGGCGGCGGCGGCT |
| | | CTGGCGGAGGCGGTAGCGCAATCCAGCTGACCCAGAGCCCTTCGAGCCTTTCTGCT |
| | | TCCGTGGGCGACAGGGTCACCATCACCTGCCGAGCGTCGCAGGGTATCTCTAGTGC |
| | | GCTGGCTTGGTACCAGCAGAAGCCTGGAAAGGCCCCCAAATTTCTCATCTACGATG |
| | | CATCTTCTCTAGAGAGCGGGGTGCCATCCCGCTTTTCAGGTTCTGGTTCCGGAACT |
| | | GATTTCACACTCACCATCAGCTCCCTGCAGCCAGAGGACTTTGCTACCTACTATTG |
| | | TCAGCAGTTCAACAGCTACCCCTTCACCTTCGGCCCGGGCACCAAGGTGGACATCA |
| | | AAGGCTCTaccaccacccctgcccctagacctcccaccccagccccaacaatcgcc |
| | | agccagcctctgtctctgcggcccgaagcctgtagacctgctgccggcggagccgt |
| | | gcacaccagaggcctggacttcgcctgcgacatctacatctgggcccctctggccg |
| | | gcacctgtggcgtgctgctgctgagcctggtgatcaccctgtactgcaaacggggc |
| | | agaaagaaactcctgtatatattcaaacaaccatttatgagaccagtacaaactac |
| | | tcaagaggaagatggctgtagctgccgatttccagaagaagaagaaggaggatgtg |
| | | aactgagagtgaagttcagcagatccgccgacgcccctgcctaccagcagggacag |
| | | aaccagctgtacaacgagctgaacctgggcagacgggaagagtacgacgtgctgga |
| | | caagcggagaggccgggaccccgagatgggcggaaagcccagacggaagaaccccc |
| | | aggaaggcctgtataacgaactgcagaaagacaagatggccgaggcctacagcgag |
| | | atcggcatgaagggcgagcggaggcgcggcaagggccacgatggcctgtaccaggg |
| | | cctgagcaccgccaccaaggacacctacgacgccctgcacatgcaggccctgcccc |
| | | ccaga TAA |
| 12 | Construct | MALPVTALLLPLALLLHAARPQIQLVESGGGVVQPGRSLRLSCAASGFTFGYYAMH |
| | 5716 CAR | WVRQAPGKGLEWVAVISYDGSIKYYADSVKGRFTISRDNSKNTLYLQMNSLRAEDT |
| | AA | AVYYCAREGPYSNYLDYWGQGTLVTVSSGGGGSGGGGSGGGGSAIQLTQSPSSLSA |
| | sequence | SVGDRVTITCRASQGISSALAWYQQKPGKAPKFLIYDASSLESGVPSRFSGSGSGT |
| | | DFTLTISSLQPEDFATYYCQQFNSYPFTFGPGTKVDIKGSTTTPAPRPPTPAPTIA |
| | | SQPLSLRPEACRPAAGGAVHTRGLDFACDIYIWAPLAGTCGVLLLSLVITLYCKRG |
| | | RKKLLYIFKQPFMRPVQTTQEEDGCSCRFPEEEEGGCELRVKFSRSADAPAYQQGQ |
| | | NQLYNELNLGRREEYDVLDKRRGRDPEMGGKPRRKNPQEGLYNELQKDKMAEAYSE |
| | | IGMKGERRRGKGHDGLYQGLSTATKDTYDALHMQALPPR* |
| 13 | Construct | ATGGCGTTGCCTGTTACCGCTCTGCTGCTGCCCCTGGCCCTGCTCTTACACGCCGC |
| | 5284 CAR | ACGTCCCCAGGTGCAACTCGTCGAAAGCGGGGGAGGGGTGGTCCAGCCGGGGCGGT |
| | ORF | CCCTACGCCTGTCATGCGCCGCTTCCGGCTTCACCTTCTCCGACCATGGAATGCAC |
| | | TGGGTCCGCCAGGCCCCGGGTAAAGGACTGGAGTGGGTGGCCGTGATTTGGTACGA |
| | | TGGCAGCAACAAGTACTACGCGGACTCAGTGAAGGGCCGCTTCACCATTTCGCGTG |
| | | ACAACTCCAAGAACACGCTTTACCTGCAGATGAACAGCCTAAGAGCTGAGGACACA |
| | | GCCGTGTACTACTGTGCGCGGGATTCTATCATGGTGCGGGGGGACTATTGGGGCCA |
| | | GGGTACACTGGTCACCGTGTCCAGTGGTGGGGGCGGTTCTGGAGGCGGAGGCTCCG |
| | | GTGGCGGCGGGAGCGACATCCAGATGACCCAGAGCCCATCTTCTCTGTCCGCTTCC |
| | | GTGGGTGATCGTGTGACGATCACCTGCCGAGCGTCGCAGGGCATCTCTAGCTGGCT |
| | | CGCCTGGTACCAACAGAAGCCGGAGAAGGCACCGAAGTCCCTGATCTACGCTGCCT |
| | | CCAGCCTGCAGAGCGGGGTACCCTCTCGCTTCTCTGGCTCTGGATCGGGCACCGAC |
| | | TTTACCCTGACTATTTCCTCCTTGCAACCCGAAGATTTCGCCACTTATTACTGTCA |
| | | GCAGTATAACAGCTACCCCCTGACTTTCGGTGGCGGCACCAAGGTGGAGATCAAGG |
| | | GCTCCACGACCACACCGGCTCCTAGGCCACCCACCCCGGCGCCAACCATCGCATCC |
| | | CAGCCTTTGAGTCTGCGTCCCGAGGCCTGTCGTCCCGCGGCTGGGGGCGCCGTCCA |
| | | CACCCGTGGTTTGGATTTCGCCTGCGACAAGGACCCGAAATTTTGGGTGCTCGTTG |
| | | TGGTGGGCGGTGTGCTTGCGTGCTACTCACTTCTGGTGACTGTTGCGTTCATCATC |
| | | TTCTGGGTCCGCTCTAAGCGCAGCCGCCTGCTGCACAGCGATTACATGAATATGAC |
| | | TCCGCGCCGCCCTGGCCCTACCCGCAAGCACTACCAGCCCTATGCTCCCCCCCGGG |
| | | ACTTTGCAGCCTACAGGTCACGCGTCAAATTTTCTCGCTCGGCGGACGCCCCTGCC |
| | | TACCAGCAGGGACAGAACCAGCTGTACAATGAACTCAACCTGGGCCGCAGAGAGGA |
| | | GTACGATGTGCTTGACAAGCGGCGCGGGAGGGACCCAGAGATGGGCGGAAAGCCTC |
| | | AGCGCCGAAAAAACCCTCAGGAGGGCCTGTACAACGAGCTGCAGAAGGACAAAATG |
| | | GCCGAGGCTTACTCCGAGATAGGTATGAAGGGCGAGCGCCGCCGCGGCAAGGGCCA |
| | | TGACGGCCTGTATCAGGGTCTCTCCACCGCTACTAAGGACACCTACGACGCTTTGC |
| | | ACATGCAGGCCCTCCCCCCACGCTAA |
| 14 | Construct | MALPVTALLLPLALLLHAARPQVQLVESGGGVVQPGRSLRLSCAASGFTFSDHGMH |
| | 5284 CAR | WVRQAPGKGLEWVAVIWYDGSNKYYADSVKGRFTISRDNSKNTLYLQMNSLRAEDT |
| | AA | AVYYCARDSIMVRGDYWGQGTLVTVSSGGGGSGGGGSGGGGSDIQMTQSPSSLSAS |
| | sequence | VGDRVTITCRASQGISSWLAWYQQKPEKAPKSLIYAASSLQSGVPSRFSGSGSGTD |
| | | FTLTISSLQPEDFATYYCQQYNSYPLTFGGGTKVEIKGSTTTPAPRPPTPAPTIAS |
| | | QPLSLRPEACRPAAGGAVHTRGLDFACDKDPKFWVLVVVGGVLACYSLLVTVAFII |
| | | FWVRSKRSRLLHSDYMNMTPRRPGPTRKHYQPYAPPRDFAAYRSRVKFSRSADAPA |
| | | YQQGQNQLYNELNLGRREEYDVLDKRRGRDPEMGGKPQRRKNPQEGLYNELQKDKM |
| | | AEAYSEIGMKGERRRGKGHDGLYQGLSTATKDTYDALHMQALPPR* |
| 15 | Construct | atggccctgcctgtgacagctctgctcctccctctggccctgctgctccatgccgc |
| | 5718 CAR | cagacccCAGGTGCAGCTGGTTGAGAGCGGTGGAGGGGTGGTGCAGCCGGGCCGCA |
| | ORF | GCCTTCGTCTGTCGTGCGCCGCATCCGGCTTCACCTTCTCTGATCATGGAATGCAC |
| | | TGGGTGCGCCAGGCCCCCGGTAAAGGCCTGGAGTGGGTCGCCGTGATCTGGTACGA |
| | | TGGCAGCAACAAGTACTACGCGGACTCCGTGAAGGGTCGTTTCACTATTAGCCGCG |
| | | ACAACTCCAAAAACACGCTGTACCTGCAGATGAATTCCCTCCGCGCCGAAGACACC |
| | | GCAGTATATTATTGTGCTCGGGACAGTATCATGGTGCGCGGCGACTATTGGGGCCA |
| | | GGGAACCCTGGTGACCGTCTCTTCTGGCGGCGGAGGCTCCGGGGGCGGCGGCTCCG |
| | | GCGGTGGGGGCTCCGACATCCAGATGACCCAGTCCCCATCGTCGCTTAGCGCCAGC |
| | | GTGGGGGACCGCGTCACCATCACCTGCCGAGCTTCTCAGGGCATCTCTAGTTGGCT |
| | | CGCGTGGTACCAGCAGAAGCCTGAGAAGGCTCCGAAGTCCTTGATCTACGCGGCTT |
| | | CCAGCCTGCAGAGCGGTGTCCCCTCTAGGTTTTCAGGCTCCGGCTCAGGTACAGAT |
| | | TTCACTCTAACTATTTCTTCCCTGCAGCCTGAGGACTTTGCCACCTACTACTGTCA |
| | | ACAGTACAACTCTTACCCCCTGACCTTCGGTGGAGGCACCAAGGTGGAGATCAAGG |
| | | GGTCGaccaccacccctgcccctagacctcccaccccagccccaacaatcgccagc |
| | | cagcctctgtctctgcggcccgaagcctgtagacctgctgccggcggagccgtgca |
| | | caccagaggcctggacttcgcctgcgacatctacatctgggcccctctggccggca |
| | | cctgtggcgtgctgctgctgagcctggtgatcaccctgtactgcaaacggggcaga |
| | | aagaaactcctgtatatattcaaacaaccatttatgagaccagtacaaactactca |
| | | agaggaagatggctgtagctgccgatttccagaagaagaagaaggaggatgtgaac |
| | | tgagagtgaagttcagcagatccgccgacgcccctgcctaccagcagggacagaac |
| | | cagctgtacaacgagctgaacctgggcagacgggaagagtacgacgtgctggacaa |
| | | gcggagaggccgggaccccgagatgggcggaaagcccagacggaagaacccccagg |
| | | aaggcctgtataacgaactgcagaaagacaagatggccgaggcctacagcgagatc |
| | | ggcatgaagggcgagcggaggcgcggcaagggccacgatggcctgtaccagggcct |
| | | gagcaccgccaccaaggacacctacgacgccctgcacatgcaggccctgcccccca |
| | | gaTAA |
| 16 | Construct | MALPVTALLLPLALLLHAARPQVQLVESGGGVVQPGRSLRLSCAASGFTFSDHGMH |
| | 5718 CAR | WVRQAPGKGLEWVAVIWYDGSNKYYADSVKGRFTISRDNSKNTLYLQMNSLRAEDT |
| | AA | AVYYCARDSIMVRGDYWGQGTLVTVSSGGGGSGGGGSGGGGSDIQMTQSPSSLSAS |
| | sequence | VGDRVTITCRASQGISSWLAWYQQKPEKAPKSLIYAASSLQSGVPSRFSGSGSGTD |
| | | FTLTISSLQPEDFATYYCQQYNSYPLTFGGGTKVEIKGSTTTPAPRPPTPAPTIAS |
| | | QPLSLRPEACRPAAGGAVHTRGLDFACDIYIWAPLAGTCGVLLLSLVITLYCKRGR |
| | | KKLLYIFKQPFMRPVQTTQEEDGCSCRFPEEEEGGCELRVKFSRSADAPAYQQGQN |
| | | QLYNELNLGRREEYDVLDKRRGRDPEMGGKPRRKNPQEGLYNELQKDKMAEAYSEI |
| | | GMKGERRRGKGHDGLYQGLSTATKDTYDALHMQALPPR* |
| 17 | Construct | atggccctgcctgtgacagctctgctcctccctctggccctgctgctccatgccgc |
| | 5714 CAR | cagacccGAGGTGCAGCTGGTGCAGAGCGGCGCCGAGGTTAAGAAGCCCGGTGCTA |
| | ORF | CAGTCAAGATCTCATGCAAGGTTTCTGGCTACACGTTCACCGACTACTACATGAAC |
| | | TGGGTCCAGCAGGCTCCGGGCAAAGGGCTGGAGTGGATGGGCATTATCAATCCATA |
| | | CAACGGTGGTACCCACTACAACCAGAAATTTAAAGGCCGTGTCACCATCACTGCGG |
| | | ACACCAGCACGGACACCGCCTACATGGAGCTGTCATCTTTGCGGTCTGAAGATACC |
| | | GCTGTATATTACTGTGCCACCTCCGGATATGACCTGTATTTCGACTATTGGGGCCA |
| | | GGGAACTCTTGTCACCGTGTCGTCCGGTGGGGGGGGCAGCGGAGGCGGTGGGAGCG |
| | | GCGGCGGTGGCTCCGAGATTGTGATGACCCAGAGCCCAGCAACCCTCTCCGTGTCT |
| | | CCCGGAGAGAGGGCCACTCTATCCTGCAAGGCATCCCAGAACGTGGGCACTGCGGT |
| | | GGCGTGGTACCAGCAGAAGCCTGGGCAGGCCCCTCGCCTCCTGATCTACTCTGCGT |
| | | TCAACCGCTACAACGGCATCCCGGCTCGCTTCTCGGGTTCGGGCCCCGGCACAGAT |
| | | TTCACCCTGACCATCTCCAGTCTGCAGAGCGAGGACTTTGCCGTGTACTACTGTCA |
| | | ACAGTACTCCACCTACCCCCTGACCTTCGGCGGAGGGACTAAGGTGGAGATCAAGG |
| | | GCTCGaccaccacccctgcccctagacctcccaccccagccccaacaatcgccagc |
| | | cagcctctgtctctgcggcccgaagcctgtagacctgctgccggcggagccgtgca |
| | | caccagaggcctggacttcgcctgcgacatctacatctgggcccctctggccggca |
| | | cctgtggcgtgctgctgctgagcctggtgatcaccctgtactgcaaacggggcaga |
| | | aagaaactcctgtatatattcaaacaaccatttatgagaccagtacaaactactca |
| | | agaggaagatggctgtagctgccgatttccagaagaagaagaaggaggatgtgaac |
| | | tgagagtgaagttcagcagatccgccgacgcccctgcctaccagcagggacagaac |
| | | cagctgtacaacgagctgaacctgggcagacgggaagagtacgacgtgctggacaa |
| | | gcggagaggccgggaccccgagatgggcggaaagcccagacggaagaacccccagg |
| | | aaggcctgtataacgaactgcagaaagacaagatggccgaggcctacagcgagatc |
| | | ggcatgaagggcgagcggaggcgcggcaagggccacgatggcctgtaccagggcct |
| | | gagcaccgccaccaaggacacctacgacgccctgcacatgcaggccctgcccccca |
| | | gaTAA |
| 18 | Construct | MALPVTALLLPLALLLHAARPEVQLVQSGAEVKKPGATVKISCKVSGYTFTDYYMN |
| | 5714 CAR | WVQQAPGKGLEWMGIINPYNGGTHYNQKFKGRVTITADTSTDTAYMELSSLRSEDT |
| | AA | AVYYCATSGYDLYFDYWGQGTLVTVSSGGGGSGGGGSGGGGSEIVMTQSPATLSVS |
| | sequence | PGERATLSCKASQNVGTAVAWYQQKPGQAPRLLIYSAFNRYNGIPARFSGSGPGTD |
| | | FTLTISSLQSEDFAVYYCQQYSTYPLTFGGGTKVEIKGSTTTPAPRPPTPAPTIAS |
| | | QPLSLRPEACRPAAGGAVHTRGLDFACDIYIWAPLAGTCGVLLLSLVITLYCKRGR |
| | | KKLLYIFKQPFMRPVQTTQEEDGCSCRFPEEEEGGCELRVKFSRSADAPAYQQGQN |
| | | QLYNELNLGRREEYDVLDKRRGRDPEMGGKPRRKNPQEGLYNELQKDKMAEAYSEI |
| | | GMKGERRRGKGHDGLYQGLSTATKDTYDALHMQALPPR* |
| 19 | Construct | ATGGCGTTGCCCGTCACCGCCCTGCTCCTGCCCCTGGCTCTGCTGCTGCACGCCGC |
| | 5283 CAR | TCGTCCTCAGGTGCAGCTTGTGGAGAGCGGAGGCGGCGTTGTGCAGCCTGGCAGAT |
| | ORF | CACTGCGCCTGTCATGCGCCACTTCCGGCTTCACCTTCTCAGACTACGGCATGCAC |
| | | TGGGTACGTCAGGCCCCCGGCAAGGGTCTGGAGTGGGTGGCGGTCATATGGTATGA |
| | | TGGGAGCAACAAGTACTACGCGGACTCCGTTAAAGGGCGTTTCACCATCTCTCGCG |
| | | ACAACTCGAAAAAGACCCTGTCCCTGCAAATGAATTCCCTGCGGGCCGAGGACACG |
| | | GCCGTGTACTACTGTGCTCGCGATTCTATCATGGTGCGGGGCGATTATTGGGGCCA |
| | | GGGGACCCTGGTGACCGTATCCTCGGGAGGTGGCGGCAGCGGAGGTGGCGGGAGTG |
| | | GCGGAGGCGGCAGTGACATCCAGATGACCCAGTCGCCTTCCTCCCTGTCCGCCTCC |
| | | GTGGGGGACAGGGTGACCATCACCTGCCGAGCATCCCAGGGCATTTCGAGTTGGCT |
| | | CGCCTGGTACCAGCAGAAGCCAGAGAAGGCCCCAAAATCCTTGATCTACGCCGCAT |
| | | CTTCCCTTCAGAGCGGAGTGCCGTCTCGCTTCAGCGGGTCTGGCTCCGGCACCGAC |
| | | TTCACTCTGACTATTAGCTCTTTACAGCCTGAAGACTTTGCTACTTATTACTGTCA |
| | | GCAGTACAACAGCTACCCCCTGACCTTCGGGGGCGGTACCAAGGTGGAGATCAAGG |
| | | GTTCCACCACAACCCCGGCTCCACGCCCCCCCACGCCCGCGCCTACCATCGCCAGC |
| | | CAGCCACTCTCTCTGCGCCCTGAGGCTTGCCGACCGGCCGCTGGGGGGGCGGTGCA |
| | | CACGCGCGGTTTGGATTTCGCGTGCGACAAGGACCCTAAGTTTTGGGTCCTGGTCG |
| | | TGGTGGGCGGCGTCCTTGCTTGTTACTCCCTACTCGTCACCGTGGCGTTCATCATC |
| | | TTCTGGGTCCGTAGCAAGCGCAGCAGGCTGCTGCACTCCGATTACATGAACATGAC |
| | | TCCGCGCCGCCCCGGTCCGACCCGCAAGCACTACCAGCCCTATGCACCGCCCAGGG |
| | | ACTTTGCTGCCTACCGATCTCGGGTCAAGTTTTCGCGCTCCGCCGACGCCCCCGCC |
| | | TACCAACAGGGACAGAACCAGCTGTACAACGAACTTAACCTGGGCCGCCGCGAGGA |
| | | GTACGATGTGCTGGACAAACGTCGCGGTCGGGACCCGGAGATGGGCGGCAAGCCTC |
| | | AGCGCCGTAAGAATCCACAGGAGGGCCTCTACAACGAGCTGCAGAAGGATAAAATG |
| | | GCCGAAGCGTATTCCGAGATTGGTATGAAGGGAGAGCGCCGCAGAGGAAAGGGCCA |
| | | TGACGGCCTGTACCAGGGTCTTAGTACAGCAACTAAGGACACCTACGACGCGCTCC |
| | | ATATGCAAGCCCTACCGCCCCGCTAA |
| 20 | Construct | MALPVTALLLPLALLLHAARPQVQLVESGGGVVQPGRSLRLSCATSGFTFSDYGMH |
| | 5283 CAR | WVRQAPGKGLEWVAVIWYDGSNKYYADSVKGRFTISRDNSKKTLSLQMNSLRAEDT |
| | AA | AVYYCARDSIMVRGDYWGQGTLVTVSSGGGGSGGGGSGGGGSDIQMTQSPSSLSAS |
| | sequence | VGDRVTITCRASQGISSWLAWYQQKPEKAPKSLIYAASSLQSGVPSRFSGSGSGTD |
| | | FTLTISSLQPEDFATYYCQQYNSYPLTFGGGTKVEIKGSTTTPAPRPPTPAPTIAS |
| | | QPLSLRPEACRPAAGGAVHTRGLDFACDKDPKFWVLVVVGGVLACYSLLVTVAFII |
| | | FWVRSKRSRLLHSDYMNMTPRRPGPTRKHYQPYAPPRDFAAYRSRVKFSRSADAPA |
| | | YQQGQNQLYNELNLGRREEYDVLDKRRGRDPEMGGKPQRRKNPQEGLYNELQKDKM |
| | | AEAYSEIGMKGERRRGKGHDGLYQGLSTATKDTYDALHMQALPPR* |
| 21 | Construct | atggccctgcctgtgacagctctgctcctccctctggccctgctgctccatgccgc |
| | 5717 CAR | cagacccCAGGTCCAGCTGGTGGAGAGTGGGGGAGGGGTGGTGCAGCCAGGGCGGT |
| | ORF | CTTTGCGCCTGAGCTGCGCCACTTCTGGCTTCACGTTCAGCGATTATGGTATGCAC |
| | | TGGGTGCGCCAGGCCCCGGGCAAAGGCCTCGAATGGGTCGCTGTGATTTGGTACGA |
| | | CGGCAGCAACAAGTACTACGCGGACAGCGTGAAGGGCCGCTTCACAATTTCTCGCG |
| | | ACAACTCCAAGAAGACTCTTTCTCTCCAGATGAATTCCCTGCGCGCCGAGGACACC |
| | | GCCGTATACTATTGTGCTCGTGATTCTATCATGGTGCGTGGTGATTATTGGGGCCA |
| | | GGGTACTCTGGTTACAGTGTCCTCCGGTGGTGGAGGCTCGGGCGGCGGAGGCTCCG |
| | | GTGGCGGCGGCTCCGACATCCAGATGACCCAGAGCCCTTCGTCGCTGTCCGCTTCC |
| | | GTGGGGGACAGGGTCACCATCACCTGCCGAGCGTCTCAGGGCATCTCGAGTTGGCT |
| | | AGCATGGTACCAGCAGAAGCCCGAGAAGGCCCCCAAAAGCCTTATCTACGCGGCTA |
| | | GCTCGTTGCAGAGCGGTGTCCCTTCCCGCTTTTCAGGCTCCGGCTCCGGCACCGAC |
| | | TTCACCCTGACCATCTCCTCTCTGCAGCCGGAGGACTTTGCCACCTACTACTGTCA |
| | | ACAGTACAACAGCTACCCCCTGACCTTCGGCGGAGGGACCAAGGTGGAGATCAAGG |
| | | GTTCCaccaccacccctgcccctagacctcccaccccagccccaacaatcgccagc |
| | | cagcctctgtctctgcggcccgaagcctgtagacctgctgccggcggagccgtgca |
| | | caccagaggcctggacttcgcctgcgacatctacatctgggcccctctggccggca |
| | | cctgtggcgtgctgctgctgagcctggtgatcaccctgtactgcaaacggggcaga |
| | | aagaaactcctgtatatattcaaacaaccatttatgagaccagtacaaactactca |
| | | agaggaagatggctgtagctgccgatttccagaagaagaagaaggaggatgtgaac |
| | | tgagagtgaagttcagcagatccgccgacgcccctgcctaccagcagggacagaac |
| | | cagctgtacaacgagctgaacctgggcagacgggaagagtacgacgtgctggacaa |
| | | gcggagaggccgggaccccgagatgggcggaaagcccagacggaagaacccccagg |
| | | aaggcctgtataacgaactgcagaaagacaagatggccgaggcctacagcgagatc |
| | | ggcatgaagggcgagcggaggcgcggcaagggccacgatggcctgtaccagggcct |
| | | gagcaccgccaccaaggacacctacgacgccctgcacatgcaggccctgcccccca |
| | | gaTAA |
| 22 | Construct | MALPVTALLLPLALLLHAARPQVQLVESGGGVVQPGRSLRLSCATSGFTFSDYGMH |
| | 5717 CAR | WVRQAPGKGLEWVAVIWYDGSNKYYADSVKGRFTISRDNSKKTLSLQMNSLRAEDT |
| | AA | AVYYCARDSIMVRGDYWGQGTLVTVSSGGGGSGGGGSGGGGSDIQMTQSPSSLSAS |
| | sequence | VGDRVTITCRASQGISSWLAWYQQKPEKAPKSLIYAASSLQSGVPSRFSGSGSGTD |
| | | FTLTISSLQPEDFATYYCQQYNSYPLTFGGGTKVEIKGSTTTPAPRPPTPAPTIAS |
| | | QPLSLRPEACRPAAGGAVHTRGLDFACDIYIWAPLAGTCGVLLLSLVITLYCKRGR |
| | | KKLLYIFKQPFMRPVQTTQEEDGCSCRFPEEEEGGCELRVKFSRSADAPAYQQGQN |
| | | QLYNELNLGRREEYDVLDKRRGRDPEMGGKPRRKNPQEGLYNELQKDKMAEAYSEI |
| | | GMKGERRRGKGHDGLYQGLSTATKDTYDALHMQALPPR* |
| 23 | Exemplary | GAGGTGCAGCTGCTAGAAAGCGGGGGGGGGCTGGTGCAGCCAGGCGGGAGCCTCCG |
| | 1F4 ORF | TCTCTCCTGCGCTGCTTCCGGCTTTACCTTTTCGATTTACGCCATGAGTTGGGTGC |
| | | GCCAGGCCCCGGGTAAGGGCCTGGAGTGGGTCTCGGCTATCAGCGATTCTGGCGGT |
| | | CGCACGTACTTCGCGGACAGCGTGCGTGGCCGGTTCACCATCTCCCGCGACAATTC |
| | | TAAGAACACGTTGTCCCTGCAGATGAACTCCCTGAGAGCCGAGGACACTGCCGTGT |
| | | ACTACTGTGCCAAGGTGGATTATAGCAACTACCTCTTTTTCGACTATTGGGGCCAG |
| | | GGCACCCTGGTGACCGTGTCGAGCGGAGGTGGAGGTTCCGGCGGGGGAGGCTCCGG |
| | | AGGCGGCGGATCTGAGATCGTGCTGACCCAGTCTCCGGGCACCCTGTCTCTGTCTC |
| | | CTGGAGAGCGCGCCACCCTGAGTTGCCGAGCTTCTCAGAGCATCAGCTCTAGCTAC |
| | | CTGGCCTGGTACCAGCAGAAGCCGGGACAGGCCCCTCGCCTGCTCATCTACGGCGC |
| | | TTCCTCGAGAGCCACTGGCATCCCTGACCGCTTCTCTGGATCGGGCTCCGGAACAG |
| | | ATTTCACTCTGACTATTTCTCGGTTGGAGCCCGAAGACTTCGCTGTCTACTATTGT |
| | | CAACAGTATGGATCCTCCCCGTACACCTTTGGTCAGGGCACCAAGCTGGAGATCAA |
| | | A |
| 24 | Exemplary | GAGGTGCAGCTCCTGGAGAGTGGTGGAGGCCTGGTCCAGCCAGGGGGCTCCTTGCG |
| | 1F4 ORF | TCTGTCTTGCGCCGCTAGCGGCTTCACTTTTTCTATCTACGCCATGAGTTGGGTGC |
| | | GCCAGGCTCCTGGTAAAGGCCTCGAGTGGGTCTCTGCCATCAGCGATTCTGGTGGA |
| | | CGCACCTACTTCGCGGACAGCGTACGTGGCCGCTTCACCATCAGCCGGGACAACTC |
| | | CAAGAACACGCTTAGCCTGCAGATGAATTCCCTCCGCGCGGAGGACACCGCAGTCT |
| | | ATTACTGTGCCAAGGTGGATTACAGCAACTACCTGTTCTTCGACTATTGGGGCCAG |
| | | GGAACATTGGTGACCGTTTCCTCTGGTGGCGGGGGTTCCGGCGGCGGCGGCTCGGG |
| | | GGGTGGGGGGTCCGAGATCGTGCTGACCCAGAGTCCTGGCACTCTTTCGCTGTCGC |
| | | CGGGGGAGCGCGCCACTCTGTCATGCCGAGCTTCTCAGTCGATCTCGTCCTCTTAC |
| | | CTGGCCTGGTACCAGCAGAAGCCCGGCCAGGCGCCGAGGCTGTTGATTTACGGCGC |
| | | ATCCTCCCGCGCTACCGGCATCCCCGACAGATTTTCAGGCAGCGGCTCCGGAACCG |
| | | ACTTCACACTAACCATTTCTCGCCTGGAACCCGAGGACTTTGCCGTGTACTACTGT |
| | | CAACAGTACGGTTCCAGCCCCTATACCTTCGGCCAGGGCACCAAGCTGGAGATCAA |
| | | G |
| 25 | 1F4 AA | EVQLLESGGGLVQPGGSLRLSCAASGFTFSIYAMSWVRQAPGKGLEWVSAISDSGG |
| | sequence | RTYFADSVRGRFTISRDNSKNTLSLQMNSLRAEDTAVYYCAKVDYSNYLFFDYWGQ |
| | | GTLVTVSSGGGGGGGGSGGGGSEIVLTQSPGTLSLSPGERATLSCRASQSISSSY |
| | | LAWYQQKPGQAPRLLIYGASSRATGIPDRFSGSGSGTDFTLTISRLEPEDFAVYYC |
| | | QQYGSSPYTFGQGTKLEIK |
| 26 | Exemplary | CAGGTGCAGCTGGTGCAGAGCGGCGCCGAGGTGAAGAAGCCCGGCTCCTCTGTGAA |
| | 8A1 ORF | AGTGTCATGCAAGGCATCTGGCTACTCCTTTTCAGATTATTACATGAACTGGGTCC |
| | | GCCAGGCCCCTGGTCAGGGACTGGAGTGGATGGGCGAGATTAATCCAACCACCGGC |
| | | GGAGCAACTTACAACCAGAAATTTAAAGCTCGGGTCACCATCACTGCGGACGAGAG |
| | | CACAAGCACAGCCTACATGGAGCTGAGCAGCTTGCGCTCGGAGGACACGGCCGTGT |
| | | ACTACTGTGCTCGTAGCCTGTTCCCCGCCAACTACGCGGGTTTCGTCTATTGGGGC |
| | | CAGGGCACTCTCGTAACCGTGTCCTCCGGAGGGGGTGGCTCCGGAGGCGGGGGGAG |
| | | TGGTGGTGGCGGCTCCGACATCCAGATGACCCAGAGCCCATCTTCCCTTTCGGCTT |
| | | CCGTGGGGGACAGGGTGACCATCACCTGCTCTGCTTCGAGTTCCGTTAGCTACATG |
| | | TATTGGTACCAGCAGAAGCCTGGCAAGGCGCCCAAGCTACTCATCTACGACACCAG |
| | | CAAGCTGGCGTCCGGTGTCCCCTCTCGCTTCTCGGGCTCCGGCTCTGGGACTGACT |
| | | ACACGTTCACCATTTCTTCCCTGCAGCCGGAAGATATCGCCACCTACTACTGTCAA |
| | | CAGTGGTCTTCCTATCCGTGGACCTTCGGACAGGGCACCAAGGTGGAGATCAAG |
| 27 | 8A1 AA | QVQLVQSGAEVKKPGSSVKVSCKASGYSFSDYYMNWVRQAPGQGLEWMGEINPTTG |
| | sequence | GATYNQKFKARVTITADESTSTAYMELSSLRSEDTAVYYCARSLFPANYAGFVYWG |
| | | QGTLVTVSSGGGGSGGGGSGGGGSDIQMTQSPSSLSASVGDRVTITCSASSSVSYM |
| | | YWYQQKPGKAPKLLIYDTSKLASGVPSRFSGSGSGTDYTFTISSLQPEDIATYYCQ |
| | | QWSSYPWTFGQGTKVEIK |
| 28 | Exemplary | CAAATTCAGCTGGTGGAGAGTGGCGGGGGCGTGGTCCAGCCGGGACGCAGCTTGCG |
| | 10B4 ORF | TCTGTCATGCGCCGCGTCTGGCTTCACTTTCGGCTACTACGCCATGCACTGGGTGC |
| | | GCCAGGCCCCCGGCAAGGGTCTTGAGTGGGTCGCCGTAATTAGCTACGACGGCTCC |
| | | ATCAAGTACTACGCGGACTCCGTCAAGGGGCGCTTCACCATATCCCGGGACAACTC |
| | | CAAGAACACGCTGTACTTGCAGATGAACTCCCTGCGCGCCGAAGATACTGCCGTGT |
| | | ACTACTGTGCTCGTGAGGGCCCCTATTCGAATTACCTGGACTATTGGGGCCAGGGG |
| | | ACCCTGGTTACCGTGTCGTCCGGGGGTGGGGGCTCCGGCGGCGGCGGCTCTGGCGG |
| | | AGGCGGTAGCGCAATCCAGCTGACCCAGAGCCCTTCGAGCCTTTCTGCTTCCGTGG |
| | | GCGACAGGGTCACCATCACCTGCCGAGCGTCGCAGGGTATCTCTAGTGCGCTGGCT |
| | | TGGTACCAGCAGAAGCCTGGAAAGGCCCCCAAATTTCTCATCTACGATGCATCTTC |
| | | TCTAGAGAGCGGGGTGCCATCCCGCTTTTCAGGTTCTGGTTCCGGAACTGATTTCA |
| | | CACTCACCATCAGCTCCCTGCAGCCAGAGGACTTTGCTACCTACTATTGTCAGCAG |
| | | TTCAACAGCTACCCCTTCACCTTCGGCCCGGGCACCAAGGTGGACATCAAAGGCTC |
| | | T |
| 29 | 10B4 AA | QIQLVESGGGVVQPGRSLRLSCAASGFTFGYYAMHWVRQAPGKGLEWVAVISYDGS |
| | sequence | IKYYADSVKGRFTISRDNSKNTLYLQMNSLRAEDTAVYYCAREGPYSNYLDYWGQG |
| | | TLVTVSSGGGGSGGGGSGGGGSAIQLTQSPSSLSASVGDRVTITCRASQGISSALA |
| | | WYQQKPGKAPKFLIYDASSLESGVPSRFSGSGSGTDFTLTISSLQPEDFATYYCQQ |
| | | FNSYPFTFGPGTKVDIKGS |
| 30 | Exemplary | CAGGTGCAACTCGTCGAAAGCGGGGGAGGGGTGGTCCAGCCGGGGCGGTCCCTACG |
| | 18E7 ORF | CCTGTCATGCGCCGCTTCCGGCTTCACCTTCTCCGACCATGGAATGCACTGGGTCC |
| | | GCCAGGCCCCGGGTAAAGGACTGGAGTGGGTGGCCGTGATTTGGTACGATGGCAGC |
| | | AACAAGTACTACGCGGACTCAGTGAAGGGCCGCTTCACCATTTCGCGTGACAACTC |
| | | CAAGAACACGCTTTACCTGCAGATGAACAGCCTAAGAGCTGAGGACACAGCCGTGT |
| | | ACTACTGTGCGCGGGATTCTATCATGGTGCGGGGGGACTATTGGGGCCAGGGTACA |
| | | CTGGTCACCGTGTCCAGTGGTGGGGGGGGTTCTGGAGGCGGAGGCTCCGGTGGCGG |
| | | CGGGAGCGACATCCAGATGACCCAGAGCCCATCTTCTCTGTCCGCTTCCGTGGGTG |
| | | ATCGTGTGACGATCACCTGCCGAGCGTCGCAGGGCATCTCTAGCTGGCTCGCCTGG |
| | | TACCAACAGAAGCCGGAGAAGGCACCGAAGTCCCTGATCTACGCTGCCTCCAGCCT |
| | | GCAGAGCGGGGTACCCTCTCGCTTCTCTGGCTCTGGATCGGGCACCGACTTTACCC |
| | | TGACTATTTCCTCCTTGCAACCCGAAGATTTCGCCACTTATTACTGTCAGCAGTAT |
| | | AACAGCTACCCCCTGACTTTCGGTGGCGGCACCAAGGTGGAGATCAAG |
| 31 | Exemplary | CAGGTGCAGCTGGTTGAGAGCGGTGGAGGGGTGGTGCAGCCGGGCCGCAGCCTTCG |
| | 18E7 ORF | TCTGTCGTGCGCCGCATCCGGCTTCACCTTCTCTGATCATGGAATGCACTGGGTGC |
| | | GCCAGGCCCCCGGTAAAGGCCTGGAGTGGGTCGCCGTGATCTGGTACGATGGCAGC |
| | | AACAAGTACTACGCGGACTCCGTGAAGGGTCGTTTCACTATTAGCCGCGACAACTC |
| | | CAAAAACACGCTGTACCTGCAGATGAATTCCCTCCGCGCCGAAGACACCGCAGTAT |
| | | ATTATTGTGCTCGGGACAGTATCATGGTGCGCGGCGACTATTGGGGCCAGGGAACC |
| | | CTGGTGACCGTCTCTTCTGGCGGCGGAGGCTCCGGGGGCGGCGGCTCCGGCGGTGG |
| | | GGGCTCCGACATCCAGATGACCCAGTCCCCATCGTCGCTTAGCGCCAGCGTGGGGG |
| | | ACCGCGTCACCATCACCTGCCGAGCTTCTCAGGGCATCTCTAGTTGGCTCGCGTGG |
| | | TACCAGCAGAAGCCTGAGAAGGCTCCGAAGTCCTTGATCTACGCGGCTTCCAGCCT |
| | | GCAGAGCGGTGTCCCCTCTAGGTTTTCAGGCTCCGGCTCAGGTACAGATTTCACTC |
| | | TAACTATTTCTTCCCTGCAGCCTGAGGACTTTGCCACCTACTACTGTCAACAGTAC |
| | | AACTCTTACCCCCTGACCTTCGGTGGAGGCACCAAGGTGGAGATCAAG |
| 32 | 18E7 AA | QVQLVESGGGVVQPGRSLRLSCAASGFTFSDHGMHWVRQAPGKGLEWVAVIWYDGS |
| | sequence | NKYYADSVKGRFTISRDNSKNTLYLQMNSLRAEDTAVYYCARDSIMVRGDYWGQGT |
| | | LVTVSSGGGGSGGGGSGGGGSDIQMTQSPSSLSASVGDRVTITCRASQGISSWLAW |
| | | YQQKPEKAPKSLIYAASSLQSGVPSRFSGSGSGTDFTLTISSLQPEDFATYYCQQY |
| | | NSYPLTFGGGTKVEIK |
| 33 | Exemplary | GAGGTGCAGCTGGTGCAGAGCGGCGCCGAGGTTAAGAAGCCCGGTGCTACAGTCAA |
| | 7F2 ORF | GATCTCATGCAAGGTTTCTGGCTACACGTTCACCGACTACTACATGAACTGGGTCC |
| | | AGCAGGCTCCGGGCAAAGGGCTGGAGTGGATGGGCATTATCAATCCATACAACGGT |
| | | GGTACCCACTACAACCAGAAATTTAAAGGCCGTGTCACCATCACTGCGGACACCAG |
| | | CACGGACACCGCCTACATGGAGCTGTCATCTTTGCGGTCTGAAGATACCGCTGTAT |
| | | ATTACTGTGCCACCTCCGGATATGACCTGTATTTCGACTATTGGGGCCAGGGAACT |
| | | CTTGTCACCGTGTCGTCCGGTGGGGGCGGCAGCGGAGGCGGTGGGAGCGGCGGCGG |
| | | TGGCTCCGAGATTGTGATGACCCAGAGCCCAGCAACCCTCTCCGTGTCTCCCGGAG |
| | | AGAGGGCCACTCTATCCTGCAAGGCATCCCAGAACGTGGGCACTGCGGTGGCGTGG |
| | | TACCAGCAGAAGCCTGGGCAGGCCCCTCGCCTCCTGATCTACTCTGCGTTCAACCG |
| | | CTACAACGGCATCCCGGCTCGCTTCTCGGGTTCGGGCCCCGGCACAGATTTCACCC |
| | | TGACCATCTCCAGTCTGCAGAGCGAGGACTTTGCCGTGTACTACTGTCAACAGTAC |
| | | TCCACCTACCCCCTGACCTTCGGCGGAGGGACTAAGGTGGAGATCAAG |
| 34 | 7F2 AA | EVQLVQSGAEVKKPGATVKISCKVSGYTFTDYYMNWVQQAPGKGLEWMGIINPYNG |
| | sequence | GTHYNQKFKGRVTITADTSTDTAYMELSSLRSEDTAVYYCATSGYDLYFDYWGQGT |
| | | LVTVSSGGGGSGGGGSGGGGSEIVMTQSPATLSVSPGERATLSCKASQNVGTAVAW |
| | | YQQKPGQAPRLLIYSAFNRYNGIPARFSGSGPGTDFTLTISSLQSEDFAVYYCQQY |
| | | STYPLTFGGGTKVEIK |
| 35 | Exemplary | CAGGTGCAGCTTGTGGAGAGCGGAGGCGGCGTTGTGCAGCCTGGCAGATCACTGCG |
| | 8B5 ORF | CCTGTCATGCGCCACTTCCGGCTTCACCTTCTCAGACTACGGCATGCACTGGGTAC |
| | | GTCAGGCCCCCGGCAAGGGTCTGGAGTGGGTGGCGGTCATATGGTATGATGGGAGC |
| | | AACAAGTACTACGCGGACTCCGTTAAAGGGCGTTTCACCATCTCTCGCGACAACTC |
| | | GAAAAAGACCCTGTCCCTGCAAATGAATTCCCTGCGGGCCGAGGACACGGCCGTGT |
| | | ACTACTGTGCTCGCGATTCTATCATGGTGCGGGGCGATTATTGGGGCCAGGGGACC |
| | | CTGGTGACCGTATCCTCGGGAGGTGGCGGCAGCGGAGGTGGCGGGAGTGGCGGAGG |
| | | CGGCAGTGACATCCAGATGACCCAGTCGCCTTCCTCCCTGTCCGCCTCCGTGGGGG |
| | | ACAGGGTGACCATCACCTGCCGAGCATCCCAGGGCATTTCGAGTTGGCTCGCCTGG |
| | | TACCAGCAGAAGCCAGAGAAGGCCCCAAAATCCTTGATCTACGCCGCATCTTCCCT |
| | | TCAGAGCGGAGTGCCGTCTCGCTTCAGCGGGTCTGGCTCCGGCACCGACTTCACTC |
| | | TGACTATTAGCTCTTTACAGCCTGAAGACTTTGCTACTTATTACTGTCAGCAGTAC |
| | | AACAGCTACCCCCTGACCTTCGGGGGCGGTACCAAGGTGGAGATCAAG |
| 36 | 8B5 AA | QVQLVESGGGVVQPGRSLRLSCATSGFTFSDYGMHWVRQAPGKGLEWVAVIWYDGS |
| | sequence | NKYYADSVKGRFTISRDNSKKTLSLQMNSLRAEDTAVYYCARDSIMVRGDYWGQGT |
| | | LVTVSSGGGGSGGGGSGGGGSDIQMTQSPSSLSASVGDRVTITCRASQGISSWLAW |
| | | YQQKPEKAPKSLIYAASSLQSGVPSRFSGSGSGTDFTLTISSLQPEDFATYYCQQY |
| | | NSYPLTFGGGTKVEIK |
| 37 | 10B4 VL | AIQLTQSPSSLSASVGDRVTITCRASQGISSALAWYQQKPGKAPKFLIYDASSLES |
| | AA | GVPSRFSGSGSGTDFTLTISSLQPEDFATYYCQQFNSYPFTFGPGTKVDIK |
| | sequence | |
| 38 | 18E7 VL | DIQMTQSPSSLSASVGDRVTITCRASQGISSWLAWYQQKPEKAPKSLIYAASSLQS |
| | AA | GVPSRFSGSGSGTDFTLTISSLQPEDFATYYCQQYNSYPLTFGGGTKVEIK |
| | sequence | |
| 39 | 1F4 VL AA | EIVLTQSPGTLSLSPGERATLSCRASQSISSSYLAWYQQKPGQAPRLLIYGASSRA |
| | sequence | TGIPDRFSGSGSGTDFTLTISRLEPEDFAVYYCQQYGSSPYTFGQGTKLEIK |
| 40 | 7F2 VL AA | EIVMTQSPATLSVSPGERATLSCKASQNVGTAVAWYQQKPGQAPRLLIYSAFNRYN |
| | sequence | GIPARFSGSGPGTDFTLTISSLQSEDFAVYYCQQYSTYPLTFGGGTKVEIK |
| 41 | 8A1 VL AA | DIQMTQSPSSLSASVGDRVTITCSASSSVSYMYWYQQKPGKAPKLLIYDTSKLASG |
| | sequence | VPSRFSGSGSGTDYTFTISSLQPEDIATYYCQQWSSYPWTFGQGTKVEIK |
| 42 | 8B5 VL AA | DIQMTQSPSSLSASVGDRVTITCRASQGISSWLAWYQQKPEKAPKSLIYAASSLQS |
| | sequence | GVPSRFSGSGSGTDFTLTISSLQPEDFATYYCQQYNSYPLTFGGGTKVEIK |
| 43 | 10B4 VH | QIQLVESGGGVVQPGRSLRLSCAASGFTFGYYAMHWVRQAPGKGLEWVAVISYDGS |
| | AA | IKYYADSVKGRFTISRDNSKNTLYLQMNSLRAEDTAVYYCAREGPYSNYLDYWGQG |
| | sequence | TLVTVSS |
| 44 | 18E7 VH | QVQLVESGGGVVQPGRSLRLSCAASGFTFSDHGMHWVRQAPGKGLEWVAVIWYDGS |
| | AA | NKYYADSVKGRFTISRDNSKNTLYLQMNSLRAEDTAVYYCARDSIMVRGDYWGQGT |
| | sequence | LVTVSS |
| 45 | 1F4 VH AA | EVQLLESGGGLVQPGGSLRLSCAASGFTFSIYAMSWVRQAPGKGLEWVSAISDSGG |
| | sequence | RTYFADSVRGRFTISRDNSKNTLSLQMNSLRAEDTAVYYCAKVDYSNYLFFDYWGQ |
| | | GTLVTVSS |
| 46 | 7F2 VH AA | EVQLVQSGAEVKKPGATVKISCKVSGYTFTDYYMNWVQQAPGKGLEWMGIINPYNG |
| | sequence | GTHYNQKFKGRVTITADTSTDTAYMELSSLRSEDTAVYYCATSGYDLYFDYWGQGT |
| | | LVTVSS |
| 47 | 8A1 VH AA | QVQLVQSGAEVKKPGSSVKVSCKASGYSFSDYYMNWVRQAPGQGLEWMGEINPTTG |
| | sequence | GATYNQKFKARVTITADESTSTAYMELSSLRSEDTAVYYCARSLFPANYAGFVYWG |
| | | QGTLVTVSS |
| 48 | 8B5 VH AA | QVQLVESGGGVVQPGRSLRLSCATSGFTFSDYGMHWVRQAPGKGLEWVAVIWYDGS |
| | sequence | NKYYADSVKGRFTISRDNSKKTLSLQMNSLRAEDTAVYYCARDSIMVRGDYWGQGT |
| | | LVTVSS |
| 49 | 10B4 VL | RASQGISSALA |
| | CDR 1 | |
| 50 | 10B4 VL | DASSLES |
| | CDR 2 | |
| 51 | 10B4 VL | QQFNSYPFT |
| | CDR 3 | |
| 52 | 18E7 VL | RASQGISSWLA |
| | CDR 1 | |
| 53 | 18E7 VL | AASSLQS |
| | CDR 2 | |
| 54 | 18E7 VL | QQYNSYPLT |
| | CDR 3 | |
| 55 | 1F4 VL | RASQSISSSYLA |
| | CDR 1 | |
| 56 | 1F4 VL | GASSRAT |
| | CDR 2 | |
| 57 | 1F4 VL | QQYGSSPYT |
| | CDR 3 | |
| 58 | 7F2 VL | SASSSVSYMY |
| | CDR 1 | |
| 59 | 7F2 VL | DTSKLAS |
| | CDR 2 | |
| 60 | 7F2 VL | QQWSSYPWT |
| | CDR 3 | |
| 61 | 8A1 VL | SASSSVSYMY |
| | CDR 1 | |
| 62 | 8A1 VL | DTSKLAS |
| | CDR 2 | |
| 63 | 8A1 VL | QQWSSYPWT |
| | CDR 3 | |
| 64 | 8B5 VL | RASQGISSWLA |
| | CDR 1 | |
| 65 | 8B5 VL | AASSLQS |
| | CDR 2 | |
| 66 | 8B5 VL | QQYNSYPLT |
| | CDR 3 | |
| 67 | 10B4 VH | YYAMH |
| | CDR 1 | |
| 68 | 10B4 VH | VISYDGSIKYYADSVKG |
| | CDR 2 | |
| 69 | 10B4 VH | EGPYSNYLDY |
| | CDR 3 | |
| 70 | 18E7 VH | DHGMH |
| | CDR 1 | |
| 71 | 18E7 VH | VIWYDGSNKYYADSVKG |
| | CDR 2 | |
| 72 | 18E7 VH | DSIMVRGDY |
| | DR: | |
| 73 | 1F4 VH | IYAMS |
| | CDR 1 | |
| 74 | 1F4 VH | AISDSGGRTYFADSVRG |
| | CDR 2 | |
| 75 | 1F4 VH | VDYSNYLFFDY |
| | CDR 3 | |
| 76 | 7F2 VH | DYYMN |
| | CDR 1 | |
| 77 | 7F2 VH | EINPTTGGATYNQKFKA |
| | CDR 2 | |
| 78 | 7F2 VH | SLFPANYAGFVY |
| | CDR 3 | |
| 79 | 8A1 VH | DYYMN |
| | CDR 1 | |
| 80 | 8A1 VH | EINPTTGGATYNQKFKA |
| | CDR 2 | |
| 81 | 8A1 VH | SLFPANYAGFVY |
| | CDR 3 | |
| 82 | 8B5 VH | DYGMH |
| | CDR 1 | |
| 83 | 8B5 VH | VIWYDGSNKYYADSVKG |
| | CDR | |
| 84 | 8B5 VH | DSIMVRGDY |
| | CDR 3 | |
| 85 | Exemplary | ACTACGACGCCCGCCCCGAGGCCCCCTACCCCCGCACCAACCATTGCGTCCCAGCC |
| | CD8a | GTTGAGCCTGCGGCCTGAAGCTTGCCGACCGGCAGCGGGTGGCGCCGTCCACACTC |
| | Hinge ORF | GCGGTTTGGATTTCGCTTGTGAC |
| 86 | Exemplary | accaccacccctgcccctagacctcccaccccagccccaacaatcgccagccagcc |
| | CD8a | tctgtctctgcggcccgaagcctgtagacctgctgccggcggagccgtgcacacca |
| | Hinge ORF | gaggcctggacttcgcctgcgac |
| 87 | Exemplary | ACGACCACACCGGCTCCTAGGCCACCCACCCCGGCGCCAACCATCGCATCCCAGCC |
| | CD8a | TTTGAGTCTGCGTCCCGAGGCCTGTCGTCCCGCGGCTGGGGGCGCCGTCCACACCC |
| | Hinge ORF | GTGGTTTGGATTTCGCCTGCGAC |
| 88 | Exemplary | ACCACAACCCCGGCTCCACGCCCCCCCACGCCCGCGCCTACCATCGCCAGCCAGCC |
| | CD8a | ACTCTCTCTGCGCCCTGAGGCTTGCCGACCGGCCGCTGGGGGGGCGGTGCACACGC |
| | Hinge ORF | GCGGTTTGGATTTCGCGTGCGAC |
| 89 | CD8a | TTTPAPRPPTPAPTIASQPLSLRPEACRPAAGGAVHTRGLDFACD |
| | Hinge AA | |
| | sequence | |
| 90 | Exemplary | TTCTGGGTACTTGTTGTGGTGGGGGGCGTATTAGCATGCTACTCCCTTCTGGTTAC |
| | CD28 TM | CGTCGCGTTCATCATCTTCTGGGTC |
| | ORF | |
| 91 | Exemplary | TTTTGGGTCCTGGTCGTGGTGGGCGGCGTCCTTGCTTGTTACTCCCTACTCGTCAC |
| | CD28 TM | CGTGGCGTTCATCATCTTCTGGGTC |
| | ORF | |
| 92 | Exemplary | TTTTGGGTGCTCGTTGTGGTGGGCGGTGTGCTTGCGTGCTACTCACTTCTGGTGAC |
| | CD28 TM | TGTTGCGTTCATCATCTTCTGGGTC |
| | ORF | |
| 93 | CD28 TM | FWVLVVVGGVLACYSLLVTVAFIIFWV |
| | AA | |
| | sequence | |
| 94 | Exemplary | atctacatctgggcccctctggccggcacctgtggcgtgctgctgctgagcctggt |
| | CD8a TM | gatcaccctgtactgc |
| | ORF | |
| 95 | CD8a TM | IYIWAPLAGTCGVLLLSLVITLYC |
| | AA | |
| | sequence | |
| 96 | Exemplary | AGGTCGAAGCGCAGCCGCCTGCTCCATAGCGACTACATGAATATGACCCCCCGTCG |
| | CD28 | GCCTGGCCCCACACGCAAGCACTACCAGCCCTACGCCCCCCCAAGAGACTTCGCGG |
| | Costim | CCTAC |
| | ORF | |
| 97 | Exemplary | CGTAGCAAGCGCAGCAGGCTGCTGCACTCCGATTACATGAACATGACTCCGCGCCG |
| | CD28 | CCCCGGTCCGACCCGCAAGCACTACCAGCCCTATGCACCGCCCAGGGACTTTGCTG |
| | Costim | CCTAC |
| | ORF | |
| 98 | Exemplary | CGCTCTAAGCGCAGCCGCCTGCTGCACAGCGATTACATGAATATGACTCCGCGCCG |
| | CD28 | CCCTGGCCCTACCCGCAAGCACTACCAGCCCTATGCTCCCCCCCGGGACTTTGCAG |
| | Costim | CCTAC |
| | ORF | |
| 99 | CD28 | RSKRSRLLHSDYMNMTPRRPGPTRKHYQPYAPPRDFAAY |
| | Costim AA | |
| | sequence | |
| 100 | Exemplary | aaacggggcagaaagaaactcctgtatatattcaaacaaccatttatgagaccagt |
| | 41BB | acaaactactcaagaggaagatggctgtagctgccgatttccagaagaagaagaag |
| | Costim | gaggatgtgaactg |
| | ORF | |
| 101 | 41BB | KRGRKKLLYIFKQPFMRPVQTTQEEDGCSCRFPEEEEGGCEL |
| | Costim AA | |
| | sequence | |
| 102 | Exemplary | CGCGTGAAGTTTTCACGCTCTGCGGACGCTCCCGCTTATCAGCAGGGCCAGAACCA |
| | CD3 Zeta | GCTTTACAACGAGCTTAACCTGGGCCGCCGAGAGGAGTACGATGTGCTGGACAAGC |
| | ORF | GCAGGGGCCGTGACCCGGAGATGGGCGGGAAGCCTCAGCGCCGCAAAAACCCACAG |
| | | GAGGGCCTGTACAACGAGCTGCAGAAGGACAAAATGGCCGAGGCCTACTCCGAGAT |
| | | AGGTATGAAGGGCGAGCGCCGGCGTGGTAAAGGCCACGATGGCCTCTATCAGGGTC |
| | | TGTCCACCGCCACCAAGGACACCTACGACGCACTGCATATGCAAGCGTTACCACCC |
| | | CGC |
| 103 | CD3 Zeta | RVKFSRSADAPAYQQGQNQLYNELNLGRREEYDVLDKRRGRDPEMGGKPRRKNPQE |
| | AA | GLYNELQKDKMAEAYSEIGMKGERRRGKGHDGLYQGLSTATKDTYDALHMQALPPR |
| | sequence | |
| 104 | Exemplary | AGAGTGAAGTTCAGCAGATCCGCCGACGCCCCTGCCTACCAGCAGGGACAGAACCA |
| | CD3 Zeta | GCTGTACAACGAGCTGAACCTGGGCAGACGGGAAGAGTACGACGTGCTGGACAAGC |
| | ORF | GGAGAGGCCGGGACCCCGAGATGGGCGGAAAGCCCAGACGGAAGAACCCCCAGGAA |
| | | GGCCTGTATAACGAACTGCAGAAAGACAAGATGGCCGAGGCCTACAGCGAGATCGG |
| | | CATGAAGGGCGAGCGGAGGCGCGGCAAGGGCCACGATGGCCTGTACCAGGGCCTGA |
| | | GCACCGCCACCAAGGACACCTACGACGCCCTGCACATGCAGGCCCTGCCCCCCAGA |
| 105 | Exemplary | GAGATCGTGCTGACCCAGAGTCCTGGCACTCTTTCGCTGTCGCCGGGGGAGCGCGC |
| | 1F4 | CACTCTGTCATGCCGAGCTTCTCAGTCGATCTCGTCCTCTTACCTGGCCTGGTACC |
| | Binder | AGCAGAAGCCCGGCCAGGCGCCGAGGCTGTTGATTTACGGCGCATCCTCCCGCGCT |
| | ORF | ACCGGCATCCCCGACAGATTTTCAGGCAGCGGCTCCGGAACCGACTTCACACTAAC |
| | | CATTTCTCGCCTGGAACCCGAGGACTTTGCCGTGTACTACTGTCAACAGTACGGTT |
| | | CCAGCCCCTATACCTTCGGCCAGGGCACCAAGCTGGAGATCAAG |
| 106 | (AAV2- | TTGGCCACTCCCTCTCTGCGCGCTCGCTCGCTCACTGAGGCCGGGCGACCAAAGGT |
| | 5ITR\| | CGCCCGACGCCCGGGCTTTGCCCGGGCGGCCTCAGTGAGCGAGCGAGCGCGCAGAG |
| | LHA\|EF1a\| | AGGGAGTGGCCAACTCCATCACTAGGGGTTCCTAGATCTTGCCAACATACCATAAA |
| | CD8a | CCTCCCATTCTGCTAATGCCCAGCCTAAGTTGGGGAGACCACTCCAGATTCCAAGA |
| | leader | TGTACAGTTTGCTTTGCTGGGCCTTTTTCCCATGCCTGCCTTTACTCTGCCAGAGT |
| | sequence\| | TATATTGCTGGGGTTTTGAAGAAGATCCTATTAAATAAAAGAATAAGCAGTATTAT |
| | 1F4-VH- | TAAGTAGCCCTGCATTTCAGGTTTCCTTGAGTGGCAGGCCAGGCCTGGCCGTGAAC |
| | VL-CD8a | GTTCACTGAAATCATGGCCTCTTGGCCAAGATTGATAGCTTGTGCCTGTCCCTGAG |
| | hingeTM- | TCCCAGTCCATCACGAGCAGCTGGTTTCTAAGATGCTATTTCCCGTATAAAGCATG |
| | 41BB-CD3z\| | AGACCGTGACTTGCCAGCCCCACAGAGCCCCGCCCTTGTCCATCACTGGCATCTGG |
| | bGH PA | ACTCCAGCCTGGGTTGGGGCAAAGAGGGAAATGAGATCATGTCCTAACCCTGATCC |
| | terminator\| | TCTTGTCCCACAGATATCCAGAACCCTGACCCTGCGGCTCCGGTGCCCGTCAGTGG |
| | RHA\| | GCAGAGCGCACATCGCCCACAGTCCCCGAGAAGTTGGGGGGAGGGGTCGGCAATTG |
| | AAV2- | AACCGGTGCCTAGAGAAGGTGGCGCGGGGTAAACTGGGAAAGTGATGTCGTGTACT |
| | 5ITR) | GGCTCCGCCTTTTTCCCGAGGGTGGGGGAGAACCGTATATAAGTGCAGTAGTCGCC |
| | | GTGAACGTTCTTTTTCGCAACGGGTTTGCCGCCAGAACACAGGTAAGTGCCGTGTG |
| | | TGGTTCCCGCGGGCCTGGCCTCTTTACGGGTTATGGCCCTTGCGTGCCTTGAATTA |
| | | CTTCCACGCCCCTGGCTGCAGTACGTGATTCTTGATCCCGAGCTTCGGGTTGGAAG |
| | | TGGGTGGGAGAGTTCGAGGCCTTGCGCTTAAGGAGCCCCTTCGCCTCGTGCTTGAG |
| | | TTGAGGCCTGGCTTGGGCGCTGGGGCCGCCGCGTGCGAATCTGGTGGCACCTTCGC |
| | | GCCTGTCTCGCTGCTTTCGATAAGTCTCTAGCCATTTAAAATTTTTGATGACCTGC |
| | | TGCGACGCTTTTTTTCTGGCAAGATAGTCTTGTAAATGCGGGCCAAGATGTGCACA |
| | | CTGGTATTTCGGTTTTTGGGGCCGCGGGCGGCGACGGGGCCCGTGCGTCCCAGCGC |
| | | ACATGTTCGGCGAGGCGGGGCCTGCGAGCGCGGCCACCGAGAATCGGACGGGGGTA |
| | | GTCTCAAGCTGGCCGGCCTGCTCTGGTGCCTGGCCTCGCGCCGCCGTGTATCGCCC |
| | | CGCCCTGGGCGGCAAGGCTGGCCCGGTCGGCACCAGTTGCGTGAGCGGAAAGATGG |
| | | CCGCTTCCCGGCCCTGCTGCAGGGAGCTCAAAATGGAGGACGCGGCGCTCGGGAGA |
| | | GCGGGCGGGTGAGTCACCCACACAAAGGAAAAGGGCCTTTCCGTCCTCAGCCGTCG |
| | | CTTCATGTGACTCCACGGAGTACCGGGCGCCGTCCAGGCACCTCGATTAGTTCTCG |
| | | AGCTTTTGGAGTACGTCGTCTTTAGGTTGGGGGGAGGGGTTTTATGCGATGGAGTT |
| | | TCCCCACACTGAGTGGGTGGAGACTGAAGTTAGGCCAGCTTGGCACTTGATGTAAT |
| | | TCTCCTTGGAATTTGCCCTTTTTGAGTTTGGATCTTGGTTCATTCTCAAGCCTCAG |
| | | ACAGTGGTTCAAAGTTTTTTTCTTCCATTTCAGGTGTCGTGATGCGGCCGCCACCA |
| | | TGGCCCTGCCTGTGACAGCTCTGCTCCTCCCTCTGGCCCTGCTGCTCCATGCCGCC |
| | | AGACCCGAGGTGCAGCTCCTGGAGAGTGGTGGAGGCCTGGTCCAGCCAGGGGGCTC |
| | | CTTGCGTCTGTCTTGCGCCGCTAGCGGCTTCACTTTTTCTATCTACGCCATGAGTT |
| | | GGGTGCGCCAGGCTCCTGGTAAAGGCCTCGAGTGGGTCTCTGCCATCAGCGATTCT |
| | | GGTGGACGCACCTACTTCGCGGACAGCGTACGTGGCCGCTTCACCATCAGCCGGGA |
| | | CAACTCCAAGAACACGCTTAGCCTGCAGATGAATTCCCTCCGCGCGGAGGACACCG |
| | | CAGTCTATTACTGTGCCAAGGTGGATTACAGCAACTACCTGTTCTTCGACTATTGG |
| | | GGCCAGGGAACATTGGTGACCGTTTCCTCTGGTGGCGGGGGTTCCGGCGGCGGCGG |
| | | CTCGGGGGGTGGGGGGTCCGAGATCGTGCTGACCCAGAGTCCTGGCACTCTTTCGC |
| | | TGTCGCCGGGGGAGCGCGCCACTCTGTCATGCCGAGCTTCTCAGTCGATCTCGTCC |
| | | TCTTACCTGGCCTGGTACCAGCAGAAGCCCGGCCAGGCGCCGAGGCTGTTGATTTA |
| | | CGGCGCATCCTCCCGCGCTACCGGCATCCCCGACAGATTTTCAGGCAGCGGCTCCG |
| | | GAACCGACTTCACACTAACCATTTCTCGCCTGGAACCCGAGGACTTTGCCGTGTAC |
| | | TACTGTCAACAGTACGGTTCCAGCCCCTATACCTTCGGCCAGGGCACCAAGCTGGA |
| | | GATCAAGGGCTCCACCACCACCCCTGCCCCTAGACCTCCCACCCCAGCCCCAACAA |
| | | TCGCCAGCCAGCCTCTGTCTCTGCGGCCCGAAGCCTGTAGACCTGCTGCCGGCGGA |
| | | GCCGTGCACACCAGAGGCCTGGACTTCGCCTGCGACATCTACATCTGGGCCCCTCT |
| | | GGCCGGCACCTGTGGCGTGCTGCTGCTGAGCCTGGTGATCACCCTGTACTGCAAAC |
| | | GGGGCAGAAAGAAACTCCTGTATATATTCAAACAACCATTTATGAGACCAGTACAA |
| | | ACTACTCAAGAGGAAGATGGCTGTAGCTGCCGATTTCCAGAAGAAGAAGAAGGAGG |
| | | ATGTGAACTGAGAGTGAAGTTCAGCAGATCCGCCGACGCCCCTGCCTACCAGCAGG |
| | | GACAGAACCAGCTGTACAACGAGCTGAACCTGGGCAGACGGGAAGAGTACGACGTG |
| | | CTGGACAAGCGGAGAGGCCGGGACCCCGAGATGGGCGGAAAGCCCAGACGGAAGAA |
| | | CCCCCAGGAAGGCCTGTATAACGAACTGCAGAAAGACAAGATGGCCGAGGCCTACA |
| | | GCGAGATCGGCATGAAGGGCGAGCGGAGGCGCGGCAAGGGCCACGATGGCCTGTAC |
| | | CAGGGCCTGAGCACCGCCACCAAGGACACCTACGACGCCCTGCACATGCAGGCCCT |
| | | GCCCCCCAGATAACCTCGACTGTGCCTTCTAGTTGCCAGCCATCTGTTGTTTGCCC |
| | | CTCCCCCGTGCCTTCCTTGACCCTGGAAGGTGCCACTCCCACTGTCCTTTCCTAAT |
| | | AAAATGAGGAAATTGCATCGCATTGTCTGAGTAGGTGTCATTCTATTCTGGGGGGT |
| | | GGGGTGGGGCAGGACAGCAAGGGGGAGGATTGGGAAGACAATAGCAGGCATGCTGG |
| | | GGATGCGGTGGGCTCTATGGCTTCTGAGGCGGAAAGAACCAGCTGGGGCTCTAGGG |
| | | GGTATCCCCACTAGTCGTGTACCAGCTGAGAGACTCTAAATCCAGTGACAAGTCTG |
| | | TCTGCCTATTCACCGATTTTGATTCTCAAACAAATGTGTCACAAAGTAAGGATTCT |
| | | GATGTGTATATCACAGACAAAACTGTGCTAGACATGAGGTCTATGGACTTCAAGAG |
| | | CAACAGTGCTGTGGCCTGGAGCAACAAATCTGACTTTGCATGTGCAAACGCCTTCA |
| | | ACAACAGCATTATTCCAGAAGACACCTTCTTCCCCAGCCCAGGTAAGGGCAGCTTT |
| | | GGTGCCTTCGCAGGCTGTTTCCTTGCTTCAGGAATGGCCAGGTTCTGCCCAGAGCT |
| | | CTGGTCAATGATGTCTAAAACTCCTCTGATTGGTGGTCTCGGCCTTATCCATTGCC |
| | | ACCAAAACCCTCTTTTTACTAAGAAACAGTGAGCCTTGTTCTGGCAGTCCAGAGAA |
| | | TGACACGGGAAAAAAGCAGATGAAGAGAAGGTGGCAGGAGAGGGCACGTGGCCCAG |
| | | CCTCAGTCTCTAGATCTAGGAACCCCTAGTGATGGAGTTGGCCACTCCCTCTCTGC |
| | | GCGCTCGCTCGCTCACTGAGGCCGCCCGGGCAAAGCCCGGGCGTCGGGCGACCTTT |
| | | GGTCGCCCGGCCTCAGTGAGCGAGCGAGCGCGCAGAGAGGGAGTGGCCAA |
| 107 | PINT005719 | GGCTCCGGTGCCCGTCAGTGGGCAGAGCGCACATCGCCCACAGTCCCCGAGAAGTT |
| | (genome | GGGGGGAGGGGTCGGCAATTGAACCGGTGCCTAGAGAAGGTGGCGCGGGGTAAACT |
| | integrated | GGGAAAGTGATGTCGTGTACTGGCTCCGCCTTTTTCCCGAGGGTGGGGGAGAACCG |
| | portion) | TATATAAGTGCAGTAGTCGCCGTGAACGTTCTTTTTCGCAACGGGTTTGCCGCCAG |
| | | AACACAGGTAAGTGCCGTGTGTGGTTCCCGCGGGCCTGGCCTCTTTACGGGTTATG |
| | | GCCCTTGCGTGCCTTGAATTACTTCCACGCCCCTGGCTGCAGTACGTGATTCTTGA |
| | | TCCCGAGCTTCGGGTTGGAAGTGGGTGGGAGAGTTCGAGGCCTTGCGCTTAAGGAG |
| | | CCCCTTCGCCTCGTGCTTGAGTTGAGGCCTGGCTTGGGCGCTGGGGCCGCCGCGTG |
| | | CGAATCTGGTGGCACCTTCGCGCCTGTCTCGCTGCTTTCGATAAGTCTCTAGCCAT |
| | | TTAAAATTTTTGATGACCTGCTGCGACGCTTTTTTTCTGGCAAGATAGTCTTGTAA |
| | | ATGCGGGCCAAGATGTGCACACTGGTATTTCGGTTTTTGGGGCCGCGGGCGGCGAC |
| | | GGGGCCCGTGCGTCCCAGCGCACATGTTCGGCGAGGCGGGGCCTGCGAGCGCGGCC |
| | | ACCGAGAATCGGACGGGGGTAGTCTCAAGCTGGCCGGCCTGCTCTGGTGCCTGGCC |
| | | TCGCGCCGCCGTGTATCGCCCCGCCCTGGGCGGCAAGGCTGGCCCGGTCGGCACCA |
| | | GTTGCGTGAGCGGAAAGATGGCCGCTTCCCGGCCCTGCTGCAGGGAGCTCAAAATG |
| | | GAGGACGCGGCGCTCGGGAGAGCGGGCGGGTGAGTCACCCACACAAAGGAAAAGGG |
| | | CCTTTCCGTCCTCAGCCGTCGCTTCATGTGACTCCACGGAGTACCGGGCGCCGTCC |
| | | AGGCACCTCGATTAGTTCTCGAGCTTTTGGAGTACGTCGTCTTTAGGTTGGGGGGA |
| | | GGGGTTTTATGCGATGGAGTTTCCCCACACTGAGTGGGTGGAGACTGAAGTTAGGC |
| | | CAGCTTGGCACTTGATGTAATTCTCCTTGGAATTTGCCCTTTTTGAGTTTGGATCT |
| | | TGGTTCATTCTCAAGCCTCAGACAGTGGTTCAAAGTTTTTTTCTTCCATTTCAGGT |
| | | GTCGTGATGCGGCCGCCACCATGGCCCTGCCTGTGACAGCTCTGCTCCTCCCTCTG |
| | | GCCCTGCTGCTCCATGCCGCCAGACCCGAGGTGCAGCTCCTGGAGAGTGGTGGAGG |
| | | CCTGGTCCAGCCAGGGGGCTCCTTGCGTCTGTCTTGCGCCGCTAGCGGCTTCACTT |
| | | TTTCTATCTACGCCATGAGTTGGGTGCGCCAGGCTCCTGGTAAAGGCCTCGAGTGG |
| | | GTCTCTGCCATCAGCGATTCTGGTGGACGCACCTACTTCGCGGACAGCGTACGTGG |
| | | CCGCTTCACCATCAGCCGGGACAACTCCAAGAACACGCTTAGCCTGCAGATGAATT |
| | | CCCTCCGCGCGGAGGACACCGCAGTCTATTACTGTGCCAAGGTGGATTACAGCAAC |
| | | TACCTGTTCTTCGACTATTGGGGCCAGGGAACATTGGTGACCGTTTCCTCTGGTGG |
| | | CGGGGGTTCCGGCGGCGGCGGCTCGGGGGGTGGGGGGTCCGAGATCGTGCTGACCC |
| | | AGAGTCCTGGCACTCTTTCGCTGTCGCCGGGGGAGCGCGCCACTCTGTCATGCCGA |
| | | GCTTCTCAGTCGATCTCGTCCTCTTACCTGGCCTGGTACCAGCAGAAGCCCGGCCA |
| | | GGCGCCGAGGCTGTTGATTTACGGCGCATCCTCCCGCGCTACCGGCATCCCCGACA |
| | | GATTTTCAGGCAGCGGCTCCGGAACCGACTTCACACTAACCATTTCTCGCCTGGAA |
| | | CCCGAGGACTTTGCCGTGTACTACTGTCAACAGTACGGTTCCAGCCCCTATACCTT |
| | | CGGCCAGGGCACCAAGCTGGAGATCAAGGGCTCCACCACCACCCCTGCCCCTAGAC |
| | | CTCCCACCCCAGCCCCAACAATCGCCAGCCAGCCTCTGTCTCTGCGGCCCGAAGCC |
| | | TGTAGACCTGCTGCCGGCGGAGCCGTGCACACCAGAGGCCTGGACTTCGCCTGCGA |
| | | CATCTACATCTGGGCCCCTCTGGCCGGCACCTGTGGCGTGCTGCTGCTGAGCCTGG |
| | | TGATCACCCTGTACTGCAAACGGGGCAGAAAGAAACTCCTGTATATATTCAAACAA |
| | | CCATTTATGAGACCAGTACAAACTACTCAAGAGGAAGATGGCTGTAGCTGCCGATT |
| | | TCCAGAAGAAGAAGAAGGAGGATGTGAACTGAGAGTGAAGTTCAGCAGATCCGCCG |
| | | ACGCCCCTGCCTACCAGCAGGGACAGAACCAGCTGTACAACGAGCTGAACCTGGGC |
| | | AGACGGGAAGAGTACGACGTGCTGGACAAGCGGAGAGGCCGGGACCCCGAGATGGG |
| | | CGGAAAGCCCAGACGGAAGAACCCCCAGGAAGGCCTGTATAACGAACTGCAGAAAG |
| | | ACAAGATGGCCGAGGCCTACAGCGAGATCGGCATGAAGGGCGAGCGGAGGCGCGGC |
| | | AAGGGCCACGATGGCCTGTACCAGGGCCTGAGCACCGCCACCAAGGACACCTACGA |
| | | CGCCCTGCACATGCAGGCCCTGCCCCCCAGATAACCTCGACTGTGCCTTCTAGTTG |
| | | CCAGCCATCTGTTGTTTGCCCCTCCCCCGTGCCTTCCTTGACCCTGGAAGGTGCCA |
| | | CTCCCACTGTCCTTTCCTAATAAAATGAGGAAATTGCATCGCATTGTCTGAGTAGG |
| | | TGTCATTCTATTCTGGGGGGTGGGGTGGGGCAGGACAGCAAGGGGGAGGATTGGGA |
| | | AGACAATAGCAGGCATGCTGGGGATGCGGTGGGCTCTATGGCTTCTGAGGCGGAAA |
| | | GAACCAGCTGGGGCTCTAGGGGGTATCCCCACTAGT |
| 108 | 5′ AAV2- | TTGGCCACTCCCTCTCTGCGCGCTCGCTCGCTCACTGAGGCCGGGCGACCAAAGGT |
| | 5ITR | CGCCCGACGCCCGGGCTTTGCCCGGGCGGCCTCAGTGAGCGAGCGAGCGCGCAGAG |
| | | AGGGAGTGGCCAACTCCATCACTAGGGGTTCCT |
| 109 | 3′ AAV2- | AGGAACCCCTAGTGATGGAGTTGGCCACTCCCTCTCTGCGCGCTCGCTCGCTCACT |
| | 5ITR: | GAGGCCGCCCGGGCAAAGCCCGGGCGTCGGGCGACCTTTGGTCGCCCGGCCTCAGT |
| | | GAGCGAGCGAGCGCGCAGAGAGGGAGTGGCCAA |
| 110 | EF1a | GGCTCCGGTGCCCGTCAGTGGGCAGAGCGCACATCGCCCACAGTCCCCGAGAAGTT |
| | | GGGGGGAGGGGTCGGCAATTGAACCGGTGCCTAGAGAAGGTGGCGCGGGGTAAACT |
| | | GGGAAAGTGATGTCGTGTACTGGCTCCGCCTTTTTCCCGAGGGTGGGGGAGAACCG |
| | | TATATAAGTGCAGTAGTCGCCGTGAACGTTCTTTTTCGCAACGGGTTTGCCGCCAG |
| | | AACACAGGTAAGTGCCGTGTGTGGTTCCCGCGGGCCTGGCCTCTTTACGGGTTATG |
| | | GCCCTTGCGTGCCTTGAATTACTTCCACGCCCCTGGCTGCAGTACGTGATTCTTGA |
| | | TCCCGAGCTTCGGGTTGGAAGTGGGTGGGAGAGTTCGAGGCCTTGCGCTTAAGGAG |
| | | CCCCTTCGCCTCGTGCTTGAGTTGAGGCCTGGCTTGGGCGCTGGGGCCGCCGCGTG |
| | | CGAATCTGGTGGCACCTTCGCGCCTGTCTCGCTGCTTTCGATAAGTCTCTAGCCAT |
| | | TTAAAATTTTTGATGACCTGCTGCGACGCTTTTTTTCTGGCAAGATAGTCTTGTAA |
| | | ATGCGGGCCAAGATGTGCACACTGGTATTTCGGTTTTTGGGGCCGCGGGCGGCGAC |
| | | GGGGCCCGTGCGTCCCAGCGCACATGTTCGGCGAGGCGGGGCCTGCGAGCGCGGCC |
| | | ACCGAGAATCGGACGGGGGTAGTCTCAAGCTGGCCGGCCTGCTCTGGTGCCTGGCC |
| | | TCGCGCCGCCGTGTATCGCCCCGCCCTGGGCGGCAAGGCTGGCCCGGTCGGCACCA |
| | | GTTGCGTGAGCGGAAAGATGGCCGCTTCCCGGCCCTGCTGCAGGGAGCTCAAAATG |
| | | GAGGACGCGGCGCTCGGGAGAGCGGGCGGGTGAGTCACCCACACAAAGGAAAAGGG |
| | | CCTTTCCGTCCTCAGCCGTCGCTTCATGTGACTCCACGGAGTACCGGGCGCCGTCC |
| | | AGGCACCTCGATTAGTTCTCGAGCTTTTGGAGTACGTCGTCTTTAGGTTGGGGGGA |
| | | GGGGTTTTATGCGATGGAGTTTCCCCACACTGAGTGGGTGGAGACTGAAGTTAGGC |
| | | CAGCTTGGCACTTGATGTAATTCTCCTTGGAATTTGCCCTTTTTGAGTTTGGATCT |
| | | TGGTTCATTCTCAAGCCTCAGACAGTGGTTCAAAGTTTTTTTCTTCCATTTCAGGT |
| | | GTCGTGA |

In some embodiments, the present disclosure provides an anti-CD70 chimeric antigen receptor (CAR) comprising: (a) an antigen-binding protein, or a fragment thereof, that binds to CD70, wherein the antigen-binding protein comprises: a heavy chain variable (VH) region comprising a complementarity determining region 1 (VH CDR1), VH CDR2, and VH CDR3, and a light chain variable (VL) region comprising a complementarity determining region 1 (VL CDR1), VL CDR2, and VL CDR3, and wherein: (i) the VH CDR1 comprises an amino acid sequence of any one of SEQ ID NOs: 67, 70, 73, 76, 79, and 82; (ii) the VH CDR2 comprises an amino acid sequence of any one of SEQ ID NOs: 68, 71, 74, 77, 80, and 83; (iii) the VH CDR3 comprises an amino acid sequence of any one of SEQ ID NOs: 69, 72, 75, 78, 81, and 84; (iv) the VL CDR1 comprises an amino acid sequence of any one of SEQ ID NOs: 49, 52, 55, 58, 61, and 64; (v) the VL CDR2 comprises an amino acid sequence of any one of SEQ ID NOs: 50, 53, 56, 59, 62, and 65; and (vi) the VL CDR3 comprises an amino acid sequence of any one of SEQ ID NOs: 51, 54, 57, 60, 63, and 66; (b) a transmembrane domain; and (c) an intracellular domain comprising a costimulatory domain comprising an amino acid sequence of SEQ ID NO: 99 or 101. In some embodiments, the present disclosure provides for a nucleic acid encoding the anti-CD70 CAR. In some embodiments, the VH CDR1, VH CDR2, VH CDR3, VL CDR1, VL CDR2, and VL CDR3 comprise the amino acid sequences of: (a) SEQ ID NOs: 73, 74, 75, 55, 56, and 57, respectively; (b) SEQ ID NOs: 67, 68, 69, 49, 50, and 51, respectively; (c) SEQ ID NOs: 70, 71, 72, 52, 53, and 54, respectively; (d) SEQ ID NOs: 76, 77, 78, 58, 59, and 60, respectively; (e) SEQ ID NOs: 79, 80, 81, 61, 62, and 63, respectively; or (f) SEQ ID NOs: 82, 83, 84, 64, 65, and 66, respectively. In some embodiments, the VH CDR1, VH CDR2, VH CDR3, VL CDR1, VL CDR2, and VL CDR3 comprise the amino acid sequences of: SEQ ID NOs: 73, 74, 75, 55, 56, and 57, respectively. In some embodiments, the VH CDR1, VH CDR2, VH CDR3, VL CDR1, VL CDR2, and VL CDR3 comprise the amino acid sequences of: SEQ ID NOs: 67, 68, 69, 49, 50, and 51, respectively. In some embodiments, the VH CDR1, VH CDR2, VH CDR3, VL CDR1, VL CDR2, and VL CDR3 comprise the amino acid sequences of: SEQ ID NOs: 70, 71, 72, 52, 53, and 54, respectively. In some embodiments, the VH CDR1, VH CDR2, VH CDR3, VL CDR1, VL CDR2, and VL CDR3 comprise the amino acid sequences of: SEQ ID NOs: 76, 77, 78, 58, 59, and 60, respectively. In some embodiments, the VH CDR1, VH CDR2, VH CDR3, VL CDR1, VL CDR2, and VL CDR3 comprise the amino acid sequences of: SEQ ID NOs: 79, 80, 81, 61, 62, and 63, respectively. In some embodiments, the VH CDR1, VH CDR2, VH CDR3, VL CDR1, VL CDR2, and VL CDR3 comprise the amino acid sequences of: SEQ ID NOs: 82, 83, 84, 64, 65, and 66, respectively.

In some embodiments, the nucleic acid encoding the anti-CD70 CAR comprises a nucleic acid sequence of any one of SEQ ID NOs: 23, 24, 26, 28, 30, 31, 33, and 35, or a nucleic acid sequence that has at least 90%, 93%, 95%, 96%, 97%, 98%, or 99% identity to any one of SEQ ID NOs: 23, 24, 26, 28, 30, 31, 33, and 35.

In some embodiments, the nucleic acid encoding the anti-CD70 CAR comprises a nucleic acid sequence of any one of SEQ ID NOs: 96-98 and 100, or a nucleic acid sequence that has at least 90%, 93%, 95%, 96%, 97%, 98%, or 99% identity to any one of SEQ ID NOs: 96-98 and 100.

In some embodiments, the nucleic acid encoding the anti-CD70 CAR comprises a hinge domain, wherein the hinge domain is encoded by a nucleic acid sequence of any one of SEQ ID NOs: 85-88 or a nucleic acid sequence that has at least 90%, 93%, 95%, 96%, 97%, 98%, or 99% identity to any one of SEQ ID NOs: 85-88.

In some embodiments, the nucleic acid encoding the anti-CD70 CAR comprises a nucleic acid sequence encoding the transmembrane domain comprising a sequence of any one of SEQ ID NOs: 90-92 and 94, or a sequence having at least 90%, 93%, 95%, 96%, 97%, 98%, or 99% identity to any one of SEQ ID NOs: 90-92 and 94.

In some embodiments, the nucleic acid encoding the anti-CD70 CAR comprises an activation domain, wherein the nucleic acid encoding the activation domain comprises a nucleic acid sequence of SEQ ID NO: 102 or 104 or a nucleic acid sequence that has at least 90%, 93%, 95%, 96%, 97%, 98%, or 99% identity to SEQ ID NO: 102 or 104.

In some embodiments, the nucleic acid encoding the anti-CD70 CAR comprises a nucleic acid sequence of any one of SEQ ID NOs: 1, 3, 5, 7, 9, 11, 13, 15, 17, 19, and 21, or a nucleic acid sequence that has at least 90%, 93%, 95%, 96%, 97%, 98%, or 99% identity to any one of SEQ ID NOs: 1, 3, 5, 7, 9, 11, 13, 15, 17, 19, and 21. In some embodiments, the nucleic acid comprises a nucleic acid sequence of SEQ ID NO: 1. In some embodiments, the nucleic acid comprises a nucleic acid sequence of SEQ ID NO: 3. In some embodiments, the nucleic acid comprises a nucleic acid sequence of SEQ ID NO: 7. In some embodiments, the disclosure provides for an mRNA encoded by the nucleic acid disclosed herein. In some embodiments, the disclosure provides for an expression vector operably linked to or comprising the nucleic acid disclosed herein.

### CD70 Binding Protein

In some embodiments, the antigen binding domain of the encoded anti-CD70 CAR comprises an antibody, an antibody fragment, an scFv, a Fv, a Fab, a (Fab′) 2, a single domain antibody (SDAB), a VH or VL domain, a camelid VHH domain or a bi-functional (e.g., bi-specific) hybrid antibody (e.g., Lanzavecchia et al., Eur. J. Immunol. 17, 105 (1987)).

In some instances, scFvs can be prepared according to method known in the art (see, for example, Bird et al, (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly4Ser) n, where n is a positive integer equal to or greater than 1. In one embodiment, the linker can be (Gly4Ser) 3 (SEQ ID NO: 940). Variation in the linker length may retain or enhance activity, giving rise to superior efficacy in activity studies.

In certain embodiments, the encoded antigen binding domain has a binding affinity KD of 10-4 M to 10-9 M. In one embodiment, the encoded CAR molecule comprises an antigen binding domain that has a binding affinity KD of 10-4 M to 10-9 M, e.g., 10-5 M to 10-7 M, e.g., 10-6 M or 10-7 M, e.g., 10-7 M to 10-8 M, e.g., 10-8 M to 10-9 M, for the target antigen.

In one aspect, the antigen binding domain of an anti-CD70 CAR of the present disclosure (e.g., an scFv) is encoded by a nucleic acid molecule whose sequence has been codon optimized for expression in a mammalian cell. In one aspect, the entire anti-CD70 CAR construct of the present disclosure is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences.

In some embodiments, the present disclosure provides an anti-CD70 chimeric antigen receptor (CAR) comprising: (a) an antigen-binding protein, or a fragment thereof, that binds to CD70, wherein the antigen-binding protein comprises: a heavy chain variable (VH) region comprising a complementarity determining region 1 (VH CDR1), VH CDR2, and VH CDR3, and a light chain variable (VL) region comprising a complementarity determining region 1 (VL CDR1), VL CDR2, and VL CDR3, and wherein: (i) the VH CDR1 comprises an amino acid sequence of any one of SEQ ID NOs: 67, 70, 73, 76, 79, and 82; (ii) the VH CDR2 comprises an amino acid sequence of any one of SEQ ID NOs: 68, 71, 74, 77, 80, and 83; (iii) the VH CDR3 comprises an amino acid sequence of any one of SEQ ID NOs: 69, 72, 75, 78, 81, and 84; (iv) the VL CDR1 comprises an amino acid sequence of any one of SEQ ID NOs: 49, 52, 55, 58, 61, and 64; (v) the VL CDR2 comprises an amino acid sequence of any one of SEQ ID NOs: 50, 53, 56, 59, 62, and 65; and (vi) the VL CDR3 comprises an amino acid sequence of any one of SEQ ID NOs: 51, 54, 57, 60, 63, and 66; (b) a transmembrane domain; and (c) an intracellular domain comprising a costimulatory domain comprising an amino acid sequence of SEQ ID NO: 99 or 101.

In some embodiments, the VH CDR1, VH CDR2, VH CDR3, VL CDR1, VL CDR2, and VL CDR3 comprise the amino acid sequences of: (a) SEQ ID NOs: 73, 74, 75, 55, 56, and 57, respectively; (b) SEQ ID NOs: 67, 68, 69, 49, 50, and 51, respectively; (c) SEQ ID NOs: 70, 71, 72, 52, 53, and 54, respectively; (d) SEQ ID NOs: 76, 77, 78, 58, 59, and 60, respectively; (e) SEQ ID NOs: 79, 80, 81, 61, 62, and 63, respectively; or (f) SEQ ID NOs: 82, 83, 84, 64, 65, and 66, respectively.

In some embodiments, the VH CDR1, VH CDR2, VH CDR3, VL CDR1, VL CDR2, and VL CDR3 comprise the amino acid sequences of: SEQ ID NOs: 73, 74, 75, 55, 56, and 57, respectively. In some embodiments, the VH CDR1, VH CDR2, VH CDR3, VL CDR1, VL CDR2, and VL CDR3 comprise the amino acid sequences of: SEQ ID NOs: 67, 68, 69, 49, 50, and 51, respectively. In some embodiments, the VH CDR1, VH CDR2, VH CDR3, VL CDR1, VL CDR2, and VL CDR3 comprise the amino acid sequences of: SEQ ID NOs: 70, 71, 72, 52, 53, and 54, respectively. In some embodiments, the VH CDR1, VH CDR2, VH CDR3, VL CDR1, VL CDR2, and VL CDR3 comprise the amino acid sequences of: SEQ ID NOs: 76, 77, 78, 58, 59, and 60, respectively. In some embodiments, the VH CDR1, VH CDR2, VH CDR3, VL CDR1, VL CDR2, and VL CDR3 comprise the amino acid sequences of: SEQ ID NOs: 79, 80, 81, 61, 62, and 63, respectively. In some embodiments, the VH CDR1, VH CDR2, VH CDR3, VL CDR1, VL CDR2, and VL CDR3 comprise the amino acid sequences of: SEQ ID NOs: 82, 83, 84, 64, 65, and 66, respectively.

In some embodiments, the VH region comprises an amino acid sequence having at least 90%, 93%, 95%, 96%, 97%, 98%, or 99% identity to any one of SEQ ID NOs: 43-48. In some embodiments, the VH region comprises the amino acid sequence of any one of SEQ ID NOs: 43-48, with 0, 1, 2, 3, 4, 5, 6, 7, 8, 9, or 10 substitutions in each of the VH CDR1, VH CDR2, and VH CDR3, independently. In some embodiments, the VL region comprises an amino acid sequence having at least 90%, 93%, 95%, 96%, 97%, 98%, or 99% identity to any one of SEQ ID NOs: 37-42. In some embodiments, the VL region comprises the amino acid sequence of any one of SEQ ID NOs: 37-42, with 0, 1, 2, 3, 4, 5, 6, 7, 8, 9, or 10 substitutions in each of the VL CDR1, VL CDR2, and VL CDR3, independently.

In some embodiments, (a) the VH region comprises an amino acid sequence having at least 90%, 93%, 95%, 96%, 97%, 98%, or 99% identity to SEQ ID NO: 45, and the VL region comprises an amino acid sequence having at least 90%, 93%, 95%, 96%, 97%, 98%, or 99% identity to SEQ ID NO: 39; (b) the VH region comprises an amino acid sequence having at least 90%, 93%, 95%, 96%, 97%, 98%, or 99% identity to SEQ ID NO: 43, and the VL region comprises an amino acid sequence having at least 90%, 93%, 95%, 96%, 97%, 98%, or 99% identity to SEQ ID NO: 37; (c) the VH region comprises an amino acid sequence having at least 90%, 93%, 95%, 96%, 97%, 98%, or 99% identity to SEQ ID NO: 44, and the VL region comprises an amino acid sequence having at least 90%, 93%, 95%, 96%, 97%, 98%, or 99% identity to SEQ ID NO: 38; (d) the VH region comprises an amino acid sequence having at least 90%, 93%, 95%, 96%, 97%, 98%, or 99% identity to SEQ ID NO: 46, and the VL region comprises an amino acid sequence having at least 90%, 93%, 95%, 96%, 97%, 98%, or 99% identity to SEQ ID NO: 40; (e) the VH region comprises an amino acid sequence having at least 90%, 93%, 95%, 96%, 97%, 98%, or 99% identity to SEQ ID NO: 47, and the VL region comprises an amino acid sequence having at least 90%, 93%, 95%, 96%, 97%, 98%, or 99% identity to SEQ ID NO: 41; or (f) the VH region comprises an amino acid sequence having at least 90%, 93%, 95%, 96%, 97%, 98%, or 99% identity to SEQ ID NO: 48, and the VL region comprises an amino acid sequence having at least 90%, 93%, 95%, 96%, 97%, 98%, or 99% identity to SEQ ID NO: 42.

In some embodiments, the VH region comprises an amino acid sequence having at least 90%, 93%, 95%, 96%, 97%, 98%, or 99% identity to SEQ ID NO: 45, and the VL region comprises an amino acid sequence having at least 90%, 93%, 95%, 96%, 97%, 98%, or 99% identity to SEQ ID NO: 39. In some embodiments, the VH region comprises an amino acid sequence having at least 90%, 93%, 95%, 96%, 97%, 98%, or 99% identity to SEQ ID NO: 43, and the VL region comprises an amino acid sequence having at least 90%, 93%, 95%, 96%, 97%, 98%, or 99% identity to SEQ ID NO: 37. In some embodiments, the VH region comprises an amino acid sequence having at least 90%, 93%, 95%, 96%, 97%, 98%, or 99% identity to SEQ ID NO: 44, and the VL region comprises an amino acid sequence having at least 90%, 93%, 95%, 96%, 97%, 98%, or 99% identity to SEQ ID NO: 38. In some embodiments, the VH region comprises an amino acid sequence having at least 90%, 93%, 95%, 96%, 97%, 98%, or 99% identity to SEQ ID NO: 46, and the VL region comprises an amino acid sequence having at least 90%, 93%, 95%, 96%, 97%, 98%, or 99% identity to SEQ ID NO: 40. In some embodiments, the VH region comprises an amino acid sequence having at least 90%, 93%, 95%, 96%, 97%, 98%, or 99% identity to SEQ ID NO: 47, and the VL region comprises an amino acid sequence having at least 90%, 93%, 95%, 96%, 97%, 98%, or 99% identity to SEQ ID NO: 41. In some embodiments, the VH region comprises an amino acid sequence having at least 90%, 93%, 95%, 96%, 97%, 98%, or 99% identity to SEQ ID NO: 48, and the VL region comprises an amino acid sequence having at least 90%, 93%, 95%, 96%, 97%, 98%, or 99% identity to SEQ ID NO: 42.

In some embodiments, (a) the VH region comprises the amino acid sequence of SEQ ID NO: 45, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 39, with 0, 1, 2, 3, 4, 5, 6, 7, 8, 9, or 10 substitutions in each of the VL CDR1, VL CDR2, and VL CDR3, independently; (b) the VH region comprises the amino acid sequence of SEQ ID NO: 43, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 37, with 0, 1, 2, 3, 4, 5, 6, 7, 8, 9, or 10 substitutions in each of the VL CDR1, VL CDR2, and VL CDR3, independently; (c) the VH region comprises the amino acid sequence of SEQ ID NO: 44, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 38, with 0, 1, 2, 3, 4, 5, 6, 7, 8, 9, or 10 substitutions in each of the VL CDR1, VL CDR2, and VL CDR3, independently; (d) the VH region comprises the amino acid sequence of SEQ ID NO: 46, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 40, with 0, 1, 2, 3, 4, 5, 6, 7, 8, 9, or 10 substitutions in each of the VL CDR1, VL CDR2, and VL CDR3, independently; (e) the VH region comprises the amino acid sequence of SEQ ID NO: 47, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 41, with 0, 1, 2, 3, 4, 5, 6, 7, 8, 9, or 10 substitutions in each of the VL CDR1, VL CDR2, and VL CDR3, independently; or (f) the VH region comprises the amino acid sequence of SEQ ID NO: 48, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 42, with 0, 1, 2, 3, 4, 5, 6, 7, 8, 9, or 10 substitutions in each of the VL CDR1, VL CDR2, and VL CDR3, independently.

In some embodiments, the VH region comprises the amino acid sequence of SEQ ID NO: 45, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 39, with 0, 1, 2, 3, 4, 5, 6, 7, 8, 9, or 10 substitutions in each of the VL CDR1, VL CDR2, and VL CDR3, independently. In some embodiments, the VH region comprises the amino acid sequence of SEQ ID NO: 43, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 37, with 0, 1, 2, 3, 4, 5, 6, 7, 8, 9, or 10 substitutions in each of the VL CDR1, VL CDR2, and VL CDR3, independently. In some embodiments, the VH region comprises the amino acid sequence of SEQ ID NO: 44, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 38, with 0, 1, 2, 3, 4, 5, 6, 7, 8, 9, or 10 substitutions in each of the VL CDR1, VL CDR2, and VL CDR3, independently. In some embodiments, the VH region comprises the amino acid sequence of SEQ ID NO: 46, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 40, with 0, 1, 2, 3, 4, 5, 6, 7, 8, 9, or 10 substitutions in each of the VL CDR1, VL CDR2, and VL CDR3, independently. In some embodiments, the VH region comprises the amino acid sequence of SEQ ID NO: 47, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 41, with 0, 1, 2, 3, 4, 5, 6, 7, 8, 9, or 10 substitutions in each of the VL CDR1, VL CDR2, and VL CDR3, independently. In some embodiments, the VH region comprises the amino acid sequence of SEQ ID NO: 48, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 42, with 0, 1, 2, 3, 4, 5, 6, 7, 8, 9, or 10 substitutions in each of the VL CDR1, VL CDR2, and VL CDR3, independently.

In some embodiments, (a) the VH region comprises the amino acid sequence of SEQ ID NO: 45, and the VL region comprises the amino acid sequence of SEQ ID NO: 39; (b) the VH region comprises the amino acid sequence of SEQ ID NO: 43, and the VL region comprises the amino acid sequence of SEQ ID NO: 37; (c) the VH region comprises the amino acid sequence of SEQ ID NO: 44, and the VL region comprises the amino acid sequence of SEQ ID NO: 38; (d) the VH region comprises the amino acid sequence of SEQ ID NO: 46, and the VL region comprises the amino acid sequence of SEQ ID NO: 40; (e) the VH region comprises the amino acid sequence of SEQ ID NO: 47, and the VL region comprises the amino acid sequence of SEQ ID NO: 41; or (f) the VH region comprises the amino acid sequence of SEQ ID NO: 48, and the VL region comprises the amino acid sequence of SEQ ID NO: 42.

In some embodiments, the VH region comprises the amino acid sequence of SEQ ID NO: 45, and the VL region comprises the amino acid sequence of SEQ ID NO: 39. In some embodiments, the VH region comprises the amino acid sequence of SEQ ID NO: 43, and the VL region comprises the amino acid sequence of SEQ ID NO: 37. In some embodiments, the VH region comprises the amino acid sequence of SEQ ID NO: 44, and the VL region comprises the amino acid sequence of SEQ ID NO: 38. In some embodiments, the VH region comprises the amino acid sequence of SEQ ID NO: 46, and the VL region comprises the amino acid sequence of SEQ ID NO: 40. In some embodiments, the VH region comprises the amino acid sequence of SEQ ID NO: 47, and the VL region comprises the amino acid sequence of SEQ ID NO: 41. In some embodiments, the VH region comprises the amino acid sequence of SEQ ID NO: 48, and the VL region comprises the amino acid sequence of SEQ ID NO: 42.

In some embodiments, the antigen-binding protein comprises an amino acid sequence of any one of SEQ ID NOs: 25, 27, 29, 32, 34, and 36, or a sequence having at least 90%, 93%, 95%, 96%, 97%, 98%, or 99% identity to any one of SEQ ID NOs: 25, 27, 29, 32, 34, and 36. In some embodiments, the antigen-binding protein comprises an amino acid sequence of SEQ ID NO: 25 or SEQ ID NO: 27. In some embodiments, the antigen-binding protein comprises an amino acid sequence having at least 90%, 93%, 95%, 96%, 97%, 98%, or 99% identity to SEQ ID NO: 25 or SEQ ID NO: 27. In some embodiments, the antigen-binding protein comprises the amino acid sequence of SEQ ID NO: 25. In some embodiments, the antigen-binding protein comprises an amino acid sequence having at least 90%, 93%, 95%, 96%, 97%, 98%, or 99% identity to SEQ ID NO: 25. In some embodiments, the antigen-binding protein comprises the amino acid sequence of SEQ ID NO: 27. In some embodiments, the antigen-binding protein comprises an amino acid sequence having at least 90%, 93%, 95%, 96%, 97%, 98%, or 99% identity to SEQ ID NO: 27.

In some embodiments, the antigen-binding protein comprises an amino acid sequence of SEQ ID NO: 25 or SEQ ID NO: 27 and the costimulatory domain comprises the amino acid sequence of SEQ ID NO: 99. In some embodiments, the antigen-binding protein comprises an amino acid sequence of SEQ ID NO: 25 and the costimulatory domain comprises the amino acid sequence of SEQ ID NO: 99. In some embodiments, the antigen-binding protein comprises an amino acid sequence of SEQ ID NO: 27 and the costimulatory domain comprises the amino acid sequence of SEQ ID NO: 99.

In some embodiments, the antigen-binding protein comprises an amino acid sequence of SEQ ID NO: 25 or SEQ ID NO: 27 and the costimulatory domain comprises the amino acid sequence of SEQ ID NO: 101. In some embodiments, the antigen-binding protein comprises an amino acid sequence of SEQ ID NO: 25 and the costimulatory domain comprises the amino acid sequence of SEQ ID NO: 101. In some embodiments, the antigen-binding protein comprises an amino acid sequence of SEQ ID NO: 27 and the costimulatory domain comprises the amino acid sequence of SEQ ID NO: 101.

In some embodiments, the transmembrane domain comprises a CD8a or CD28 transmembrane domain. In some embodiments, the transmembrane domain comprises a CD8a transmembrane domain comprising an amino acid sequence of SEQ ID NO: 95. In some embodiments, the transmembrane domain comprises a CD8a transmembrane region comprising an amino acid sequence having at least 90%, 93%, 95%, 96%, 97%, 98%, or 99% identity to SEQ ID NO: 95. In some embodiments, the transmembrane domain comprises a CD28 transmembrane domain comprising an amino acid sequence of SEQ ID NO: 93. In some embodiments, the transmembrane domain comprises a CD28 transmembrane domain having at least 90%, 93%, 95%, 96%, 97%, 98%, or 99% identity to SEQ ID NO: 93.

In some embodiments, the anti-CD70 CAR further comprises a hinge domain between the antigen binding protein and the transmembrane domain. In some embodiments, the anti-CD70 CAR comprises a linker between the anti-CD70 binding protein (e.g., scFv) and the hinge domain. In some embodiments, the linker sequence is GlySer. In some embodiments, the hinge domain is a CD8a hinge domain. In some embodiments, the hinge domain is a CD8a hinge domain, or fragment thereof, comprising an amino acid sequence of SEQ ID NO: 89. In some embodiments, the hinge domain is a CD8a hinge domain, or fragment thereof, comprising an amino acid sequence having at least 90%, 93%, 95%, 96%, 97%, 98%, or 99% identity to SEQ ID NO: 89.

In some embodiments, the intracellular domain further comprises an activation domain. In some embodiments, the activation domain is a CD3z activation domain comprising an amino acid sequence of SEQ ID NO: 103. In some embodiments, the hinge domain is a CD3z hinge domain comprising an amino acid sequence having at least 90%, 93%, 95%, 96%, 97%, 98%, or 99% identity to SEQ ID NO: 103.

In some embodiments, the intracellular domain comprises a CD28 costimulatory domain comprising the amino acid sequence of SEQ ID NO: 99 and a CD3z activation domain comprising the amino acid sequence of SEQ ID NO: 103. In some embodiments, the intracellular domain comprises a 41BB costimulatory domain comprising the amino acid sequence of SEQ ID NO: 101 and a CD3z activation domain comprising the amino acid sequence of SEQ ID NO: 103.

In some embodiments, the antigen-binding protein comprises a sequence of SEQ ID NO: 32 and the costimulatory domain comprises a sequence of SEQ ID NO: 101; the antigen-binding protein comprises a sequence of SEQ ID NO: 32 and the costimulatory domain comprises a sequence of SEQ ID NO: 99; the antigen-binding protein comprises a sequence of SEQ ID NO: 25 and the costimulatory domain comprises a sequence of SEQ ID NO: 101; the antigen-binding protein comprises a sequence of SEQ ID NO: 25 and the costimulatory domain comprises a sequence of SEQ ID NO: 99; the antigen-binding protein comprises a sequence of SEQ ID NO: 27 and the costimulatory domain comprises a sequence of SEQ ID NO: 101; the antigen-binding protein comprises a sequence of SEQ ID NO: 27 and the costimulatory domain comprises a sequence of SEQ ID NO: 99; the antigen-binding protein comprises a sequence of SEQ ID NO: 36 and the costimulatory domain comprises a sequence of SEQ ID NO: 101; or the antigen-binding protein comprises a sequence of SEQ ID NO: 36 and the costimulatory domain comprises a sequence of SEQ ID NO: 99.

In some embodiments, the anti-CD70 CAR comprises an amino acid sequence that is at least 95% identical to the amino acid sequence of any one of SEQ ID NOs: 2, 4, 6, 8, 14, 16, 20, and 22. In one embodiment, the anti-CD70 CAR comprises an amino acid sequence that is at least 95% identical to the amino acid sequence of SEQ ID NO: 2. In one embodiment, the anti-CD70 CAR comprises an amino acid sequence that is at least 95% identical to the amino acid sequence of SEQ ID NO: 4. In one embodiment, the anti-CD70 CAR comprises an amino acid sequence that is at least 95% identical to the amino acid sequence of SEQ ID NO: 6. In one embodiment, the anti-CD70 CAR comprises an amino acid sequence that is at least 95% identical to the amino acid sequence of SEQ ID NO: 8. In one embodiment, the anti-CD70 CAR comprises an amino acid sequence that is at least 95% identical to the amino acid sequence of SEQ ID NO:14. In one embodiment, the anti-CD70 CAR comprises an amino acid sequence that is at least 95% identical to the amino acid sequence of SEQ ID NO: 16. In one embodiment, the anti-CD70 CAR comprises an amino acid sequence that is at least 95% identical to the amino acid sequence of SEQ ID NO: 20. In one embodiment, the anti-CD70 CAR comprises an amino acid sequence that is at least 95% identical to the amino acid sequence of SEQ ID NO: 22.

In some embodiments, the antigen-binding protein is an scFv.

Preferably, the disclosed anti-CD70 CARs comprise an scFv comprising the CDR sequences corresponding to the light and heavy chain variable regions of an anti-CD70 antibody. Single chain antibodies may be formed by linking heavy and light chain variable domain (Fv region) fragments via an amino acid bridge (short peptide linker), resulting in a single polypeptide chain. Such single-chain Fvs (scFvs) have been prepared by fusing DNA encoding a peptide linker between DNAs encoding the two variable domain polypeptides (VL and VH). The resulting polypeptides can fold back on themselves to form antigen-binding monomers, or they can form multimers (e.g., dimers, trimers, or tetramers), depending on the length of a flexible linker between the two variable domains (Kortt et al., 1997, Prot. Eng. 10:423; Kortt et al., 2001, Biomol. Eng. 18:95-108). By combining different VL- and VH-comprising polypeptides, one can form multimeric scFvs that bind to different epitopes (Kriangkum et al., 2001, Biomol. Eng. 18:31-40). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, and de Graaf et al., 2002, Methods Mol. Biol. 178:379-87.

Preferably, the disclosed anti-CD70 CARs comprise an antigen-binding protein comprising an amino acid sequence that is at least at least 90%, 93%, 95%, 96%, 97%, 98%, or 99% identical to an amino acid sequence selected from any one of SEQ ID NOs: 25, 27, 29, 32, 34, and 36. In some embodiments, the antigen-binding protein comprises an amino acid sequence selected from any one of SEQ ID NOs: 25, 27, 29, 32, 34, and 36.

### Hinge Domains

The anti-CD70 CARs described herein further comprise a hinge domain. The hinge domain is located between the antigen-binding region and the transmembrane domain. A hinge domain is an amino acid segment that is generally found between two domains of a protein, e.g., a human protein, and, in the context of anti-CD70 CARs, permits the movement of one or both of the antigen-binding region and transmembrane domain relative to each other. Preferably, the hinge domain comprises from about 10 to about 100 amino acids, e.g., from about 15 to about 75 amino acids, from about 20 to about 50 amino acids, or from about 30 to about 60 amino acids. In some embodiments, the hinge domain is a hinge domain of a naturally occurring protein.

Preferably, the hinge domain used in a CAR is derived from CD8a. Preferably, the hinge domain of an anti-CD70 CAR comprises an amino acid sequence that is at least 90%, 93%, 95%, 96%, 97%, 98%, or 99% identical to an amino acid sequence of SEQ ID NO: 89. In some embodiments, the hinge domain comprises an amino acid sequence of SEQ ID NO: 89.

Preferably, the hinge domain is positioned between the C-terminus of the scFv and the N-terminus of the transmembrane domain of the anti-CD70 CAR.

### Transmembrane Domains

With respect to the transmembrane domain, in various embodiments, a CAR can be designed to comprise a transmembrane domain that is attached to the extracellular domain of the CAR. A transmembrane domain can include one or more additional amino acids adjacent to the transmembrane domain, e.g., one or more amino acid associated with the extracellular region of the protein from which the transmembrane domain was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR e.g., in one embodiment, the transmembrane domain may be from the same protein that the intracellular domain, costimulatory domain or the hinge domain is derived from. In another aspect, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target.

In some embodiments, the transmembrane domain may be recombinant, in which case it will comprise predominantly hydrophobic residues such as leucine and valine. In one aspect a triplet of phenylalanine, tryptophan and valine can be found at each end of a recombinant transmembrane domain. Optionally, a short oligo- or polypeptide linker, between 2 and 10 amino acids in length may form the linkage between the transmembrane domain and the cytoplasmic region of the CAR. A glycine-serine doublet provides a particularly suitable linker.

Preferably, the transmembrane domain used in a CAR is derived from a membrane protein selected from CD8a and CD28. Preferably, the transmembrane domain of an anti-CD70 CAR comprises an amino acid sequence that is at least 90%, 93%, 95%, 96%, 97%, 98%, or 99% identical to an amino acid sequence selected from SEQ ID NO: 93 and 95. In some embodiments, the transmembrane domain comprises an amino acid sequence of SEQ ID NO: 93 or 95.

In some embodiments, the transmembrane domain is a CD8a transmembrane domain comprising an amino acid sequence of SEQ ID NO: 95. In some embodiments, the transmembrane domain is a CD28 transmembrane domain comprising an amino acid sequence of SEQ ID NO: 93.

### Intracellular Domains

In some embodiments of the present disclosure having an intracellular domain, such a domain can contain, e.g., one or more of an activation domain and/or a costimulatory domain. In some embodiments, the intracellular domain comprises a sequence encoding an activation domain. In some embodiments, the intracellular domain comprises a costimulatory domain. In some embodiments, the intracellular domain comprises an activation domain and a costimulatory domain.

The intracellular domain sequences within the cytoplasmic portion of the CAR of the present disclosure may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences. In one embodiment, a glycine-serine doublet can be used as a suitable linker. In one embodiment, a single amino acid, e.g., an alanine, a glycine, can be used as a suitable linker.

In one aspect, the intracellular domain is designed to comprise two or more, e.g., 2, 3, 4, 5, or more, costimulatory domains. In an embodiment, the two or more, e.g., 2, 3, 4, 5, or more, costimulatory domains, are separated by a linker molecule, e.g., a linker molecule described herein. In one embodiment, the intracellular domain comprises two costimulatory domains. In some embodiments, the linker molecule is a glycine residue. In some embodiments, the linker is an alanine residue.

### 1. Activation Domain

In some embodiments, the disclosed anti-CD70 CARs comprise an intracellular activation domain. The activation domain is generally responsible for activation of at least one of the normal effector functions of a cell. The term “effector function” describes a specialized function of a cell. For example, the effector function of a T-cell or an NK cell includes a cytolytic activity or helper activity. “Activation domain” describes the portion of a protein which transduces the effector function signal and directs the cell to perform its specialized function. While the entire activation domain can be employed, in many cases it is not necessary to use an entire chain or domain. To the extent that a truncated portion of the activation domain is used, such truncated portion may be used in place of the intact domain as long as it transduces the effector function signal.

An activation domain promotes the activation of the TCR complex. Activation domains may contain signaling motifs which are known as immunoreceptor tyrosine-based activation motifs (ITAMs). Activation domains containing ITAMs for use in the anti-CD70 CARs include the intracellular domains of TCR zeta, FcR gamma, FcR beta, CD3 gamma, CD3 delta, CD3 epsilon, CD5, CD22, CD79a, CD79b, and CD66d. Preferably, an activation domain is CD33 or CD28.

Preferably, the activation domain used in a CAR is derived from a membrane protein selected from CD3z. Preferably, the activation domain of an anti-CD70 CAR comprises an amino acid sequence that is at least 90%, 93%, 95%, 96%, 97%, 98%, or 99% identical to SEQ ID NO: 103. In some embodiments, the activation domain comprises an amino acid sequence of SEQ ID NO: 103.

In some embodiments, the intracellular domain comprises an activation domain, or a functional fragment thereof, the activation domain is a CD3z activation domain comprising an amino acid sequence of SEQ ID NO: 103.

### 2. Costimulatory Domain

In some embodiments, the disclosed anti-CD70 CARs comprise a costimulatory domain. Examples of costimulatory domains for use in the chimeric receptors are cytoplasmic signaling domain of costimulatory proteins selected from the group consisting of members of the B7/CD28 family (B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (4-1BB/TNFSF9/CD137, 4-1BB ligand/TNFSF9, BAFF/BLyS/TNFSF13B, BAFF R/TNFRSF13C, CD27/TNFRSF7, CD27 ligand/TNFSF7, CD30/TNFRSF8, CD30 ligand/TNFSF8, CD40/TNFRSF5, CD40/TNFSF5, CD40 ligand/TNFSF5, DR3/TNFRSF25, GITR/TNFRSF18, GITR ligand/TNFSF18, HVEM/TNFRSF14, LIGHT/TNFSF14, lymphotoxin-alpha/TNF-beta, OX40/TNFRSF4, OX40 ligand/TNFSF4, RELT/TNFRSF19L, TACI/TNFRSF13B, TL1A/TNFSF15, TNF-α, and TNF RII/TNFRSF1B); members of the interleukin-1 receptor/toll-like receptor (TLR) superfamily (TLR1, TLR2, TLR3, TLR4, TLR5, TLR6, TLR7, TLR8, TLR9, and TLR10); members of the SLAM family (2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); CD2, CD7, CD53, CD82/Kai-1, CD90/Thyl, CD96, CD160, CD200, CD300a/LMIR1, HLA Class I, HLA-DR, ikaros, integrin alpha 4/CD49d, integrin alpha 4 beta 1, integrin alpha 4 beta 7/LPAM-1, LAG-3, TCL1A, TCL1B, CRTAM, DAP10, DAP12, MYD88, TRIF, TIRAP, TRAF, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. Preferably, the costimulatory domain comprises an intracellular domain of an activating receptor protein selected from the group consisting of a4β1 integrin, β2 integrins (CD11a-CD18, CD11b-CD18, CD11b-CD18), CD226, CRTAM, CD27, NKp46, CD16, NKp30, NKp44, NKp80, NKG2D, KIR-S, CD100, CD94/NKG2C, CD94/NKG2E, NKG2D, PEN5, CEACAM1, BY55, CRACC, Ly9, CD84, NTBA, 2B4, SAP, DAP10, DAP12, EAT2, FcRγ, CD3ζ, and ERT. Preferably, the costimulatory domain comprises an intracellular domain of an inhibitory receptor protein selected from the group consisting of KIR-L, LILRB1, CD94/NKG2A, KLRG-1, NKR-PIA, TIGIT, CEACAM, SIGLEC 3, SIGLEC 7, SIGLEC9, and LAIR-1. Preferably, the costimulatory domain comprises an intracellular domain of a protein selected from the group consisting of CD27, CD28, 4-1BB (CD137), OX40, CD30, CD40, PD1, ICOS, lymphocyte function-associated antigen-1 (LFA-1), CD2, CD7, LIGHT, NKG2C, B7-H3, and a ligand that specifically binds with CD83.

Preferably, the costimulatory domain of the anti-CD70 CAR disclosed herein is derived from a membrane protein selected from 4-1BB and CD28. Preferably, the costimulatory domain of an anti-CD70 CAR comprises an amino acid sequence that is at least 90%, 93%, 95%, 96%, 97%, 98%, or 99% identical to an amino acid sequence of SEQ ID NO: 99 or 101. In some embodiments, the costimulatory domain comprises an amino acid sequence of SEQ ID NO: 99 or 101. In some embodiments, the costimulatory domain of the CAR disclosed herein comprises a costimulatory domain of 4-1BB. In some embodiments, the costimulatory domain of the CAR disclosed herein comprises a costimulatory domain of CD28.

In some embodiments, the intracellular domain comprises a costimulatory domain, or a functional fragment thereof, the costimulatory domain is a 4-1BB or CD28 costimulatory domain. In some embodiments, the costimulatory domain is a 4-1BB costimulatory domain comprising an amino acid sequence of SEQ ID NO: 101. In some embodiments, the costimulatory domain is a CD28 costimulatory domain comprising an amino acid sequence of SEQ ID NO: 99.

### Vectors Comprising Anti-CD70 CARs

In another aspect, the present disclosure pertains to a vector comprising a nucleic acid sequence encoding an anti-CD70 CAR described herein. In one embodiment, the vector is chosen from a DNA vector, an RNA vector, a plasmid, a lentivirus vector, adenoviral vector, and a retrovirus vector. In one embodiment, the vector is a lentivirus vector. These vectors or portions thereof may, among other things, be used to create template nucleic acids, as described herein for use with the CRISPR systems as described herein. Alternatively, the vectors may be used to deliver nucleic acid directly to the cell, e.g., the immune effector cell, e.g., the T-cell, e.g., the allogeneic T-cell, independent of the CRISPR system.

The present disclosure also provides vectors in which a DNA of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (ψ), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3 (6): 677-713.

In another embodiment, the vector comprising the nucleic acid encoding the desired CAR of the present disclosure is an adenoviral vector (A5/35). In another embodiment, the expression of nucleic acids encoding CARs can be accomplished using of transposons such as sleeping beauty, crisper, CAS9, and zinc finger nucleases. See below June et al. 2009Nature Reviews Immunology 9.10:704-716, is incorporated herein by reference.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Disclosed herein are methods for producing an in vitro transcribed RNA encoding an anti-CD70 CAR. The present disclosure also includes a CAR encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a poly-A tail, typically 50-2000 bases in length. RNA so produced can efficiently transfect different kinds of cells. In one aspect, the template includes sequences for the anti-CD70 CAR.

### III. Genetically Modified Cells Comprising Anti-CD70 Chimeric Antigen Receptors (CARs) and Reduced or Eliminated Surface Expression of One or More of HLA-A, HLA-B, TRAC, MHC Class II, TGFBR2, and CD70

### Engineered Cell Compositions

In another aspect, the present disclosure provides for engineered cells or cell populations that comprising a CAR, e.g., a CAR as described in Section II. In some embodiments, the cell is engineered to express an anti-CD70 CAR, e.g., as described herein. In some embodiments, the CAR-engineered cell is allogeneic. In embodiments, the CAR-engineered cell is autologous.

In some embodiments, the present disclosure provides an anti-CD70 chimeric antigen receptor (CAR) comprising: (a) an antigen-binding protein, or a fragment thereof, that binds to CD70, wherein the antigen-binding protein comprises: a heavy chain variable (VH) region comprising a complementarity determining region 1 (VH CDR1), VH CDR2, and VH CDR3, and a light chain variable (VL) region comprising a complementarity determining region 1 (VL CDR1), VL CDR2, and VL CDR3, and wherein: (i) the VH CDR1 comprises an amino acid sequence of any one of SEQ ID NOs: 67, 70, 73, 76, 79, and 82; (ii) the VH CDR2 comprises an amino acid sequence of any one of SEQ ID NOs: 68, 71, 74, 77, 80, and 83; (iii) the VH CDR3 comprises an amino acid sequence of any one of SEQ ID NOs: 69, 72, 75, 78, 81, and 84; (iv) the VL CDR1 comprises an amino acid sequence of any one of SEQ ID NOs: 49, 52, 55, 58, 61, and 64; (v) the VL CDR2 comprises an amino acid sequence of any one of SEQ ID NOs: 50, 53, 56, 59, 62, and 65; and (vi) the VL CDR3 comprises an amino acid sequence of any one of SEQ ID NOs: 51, 54, 57, 60, 63, and 66; (b) a transmembrane domain; and (c) an intracellular domain comprising a costimulatory domain comprising an amino acid sequence of SEQ ID NO: 99 or 101.

In some embodiments, the VH CDR1, VH CDR2, VH CDR3, VL CDR1, VL CDR2, and VL CDR3 comprise the amino acid sequences of: (a) SEQ ID NOs: 73, 74, 75, 55, 56, and 57, respectively; (b) SEQ ID NOs: 67, 68, 69, 49, 50, and 51, respectively; (c) SEQ ID NOs: 70, 71, 72, 52, 53, and 54, respectively; (d) SEQ ID NOs: 76, 77, 78, 58, 59, and 60, respectively; (e) SEQ ID NOs: 79, 80, 81, 61, 62, and 63, respectively; or (f) SEQ ID NOs: 82, 83, 84, 64, 65, and 66, respectively.

In some embodiments, the VH CDR1, VH CDR2, VH CDR3, VL CDR1, VL CDR2, and VL CDR3 comprise the amino acid sequences of: SEQ ID NOs: 73, 74, 75, 55, 56, and 57, respectively. In some embodiments, the VH CDR1, VH CDR2, VH CDR3, VL CDR1, VL CDR2, and VL CDR3 comprise the amino acid sequences of: SEQ ID NOs: 67, 68, 69, 49, 50, and 51, respectively. In some embodiments, the VH CDR1, VH CDR2, VH CDR3, VL CDR1, VL CDR2, and VL CDR3 comprise the amino acid sequences of: SEQ ID NOs: 70, 71, 72, 52, 53, and 54, respectively. In some embodiments, the VH CDR1, VH CDR2, VH CDR3, VL CDR1, VL CDR2, and VL CDR3 comprise the amino acid sequences of: SEQ ID NOs: 76, 77, 78, 58, 59, and 60, respectively. In some embodiments, the VH CDR1, VH CDR2, VH CDR3, VL CDR1, VL CDR2, and VL CDR3 comprise the amino acid sequences of: SEQ ID NOs: 79, 80, 81, 61, 62, and 63, respectively. In some embodiments, the VH CDR1, VH CDR2, VH CDR3, VL CDR1, VL CDR2, and VL CDR3 comprise the amino acid sequences of: SEQ ID NOs: 82, 83, 84, 64, 65, and 66, respectively.

In some embodiments, the VH region comprises an amino acid sequence having at least 90%, 93%, 95%, 96%, 97%, 98%, or 99% identity to any one of SEQ ID NOs: 43-48. In some embodiments, the VH region comprises the amino acid sequence of any one of SEQ ID NOs: 43-48, with 0, 1, 2, 3, 4, 5, 6, 7, 8, 9, or 10 substitutions in each of the VH CDR1, VH CDR2, and VH CDR3, independently. In some embodiments, the VL region comprises an amino acid sequence having at least 90%, 93%, 95%, 96%, 97%, 98%, or 99% identity to any one of SEQ ID NOs: 37-42. In some embodiments, the VL region comprises the amino acid sequence of any one of SEQ ID NOs: 37-42, with 0, 1, 2, 3, 4, 5, 6, 7, 8, 9, or 10 substitutions in each of the VL CDR1, VL CDR2, and VL CDR3, independently.

In some embodiments, (a) the VH region comprises an amino acid sequence having at least 90%, 93%, 95%, 96%, 97%, 98%, or 99% identity to SEQ ID NO: 45, and the VL region comprises an amino acid sequence having at least 90%, 93%, 95%, 96%, 97%, 98%, or 99% identity to SEQ ID NO: 39; (b) the VH region comprises an amino acid sequence having at least 90%, 93%, 95%, 96%, 97%, 98%, or 99% identity to SEQ ID NO: 43, and the VL region comprises an amino acid sequence having at least 90%, 93%, 95%, 96%, 97%, 98%, or 99% identity to SEQ ID NO: 37; (c) the VH region comprises an amino acid sequence having at least 90%, 93%, 95%, 96%, 97%, 98%, or 99% identity to SEQ ID NO: 44, and the VL region comprises an amino acid sequence having at least 90%, 93%, 95%, 96%, 97%, 98%, or 99% identity to SEQ ID NO: 38; (d) the VH region comprises an amino acid sequence having at least 90%, 93%, 95%, 96%, 97%, 98%, or 99% identity to SEQ ID NO: 46, and the VL region comprises an amino acid sequence having at least 90%, 93%, 95%, 96%, 97%, 98%, or 99% identity to SEQ ID NO: 40; (e) the VH region comprises an amino acid sequence having at least 90%, 93%, 95%, 96%, 97%, 98%, or 99% identity to SEQ ID NO: 47, and the VL region comprises an amino acid sequence having at least 90%, 93%, 95%, 96%, 97%, 98%, or 99% identity to SEQ ID NO: 41; or (f) the VH region comprises an amino acid sequence having at least 90%, 93%, 95%, 96%, 97%, 98%, or 99% identity to SEQ ID NO: 48, and the VL region comprises an amino acid sequence having at least 90%, 93%, 95%, 96%, 97%, 98%, or 99% identity to SEQ ID NO: 42.

In some embodiments, the VH region comprises an amino acid sequence having at least 90%, 93%, 95%, 96%, 97%, 98%, or 99% identity to SEQ ID NO: 45, and the VL region comprises an amino acid sequence having at least 90%, 93%, 95%, 96%, 97%, 98%, or 99% identity to SEQ ID NO: 39. In some embodiments, the VH region comprises an amino acid sequence having at least 90%, 93%, 95%, 96%, 97%, 98%, or 99% identity to SEQ ID NO: 43, and the VL region comprises an amino acid sequence having at least 90%, 93%, 95%, 96%, 97%, 98%, or 99% identity to SEQ ID NO: 37. In some embodiments, the VH region comprises an amino acid sequence having at least 90%, 93%, 95%, 96%, 97%, 98%, or 99% identity to SEQ ID NO: 44, and the VL region comprises an amino acid sequence having at least 90%, 93%, 95%, 96%, 97%, 98%, or 99% identity to SEQ ID NO: 38. In some embodiments, the VH region comprises an amino acid sequence having at least 90%, 93%, 95%, 96%, 97%, 98%, or 99% identity to SEQ ID NO: 46, and the VL region comprises an amino acid sequence having at least 90%, 93%, 95%, 96%, 97%, 98%, or 99% identity to SEQ ID NO: 40. In some embodiments, the VH region comprises an amino acid sequence having at least 90%, 93%, 95%, 96%, 97%, 98%, or 99% identity to SEQ ID NO: 47, and the VL region comprises an amino acid sequence having at least 90%, 93%, 95%, 96%, 97%, 98%, or 99% identity to SEQ ID NO: 41. In some embodiments, the VH region comprises an amino acid sequence having at least 90%, 93%, 95%, 96%, 97%, 98%, or 99% identity to SEQ ID NO: 48, and the VL region comprises an amino acid sequence having at least 90%, 93%, 95%, 96%, 97%, 98%, or 99% identity to SEQ ID NO: 42.

In some embodiments, (a) the VH region comprises the amino acid sequence of SEQ ID NO: 45, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 39, with 0, 1, 2, 3, 4, 5, 6, 7, 8, 9, or 10 substitutions in each of the VL CDR1, VL CDR2, and VL CDR3, independently; (b) the VH region comprises the amino acid sequence of SEQ ID NO: 43, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 37, with 0, 1, 2, 3, 4, 5, 6, 7, 8, 9, or 10 substitutions in each of the VL CDR1, VL CDR2, and VL CDR3, independently; (c) the VH region comprises the amino acid sequence of SEQ ID NO: 44, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 38, with 0, 1, 2, 3, 4, 5, 6, 7, 8, 9, or 10 substitutions in each of the VL CDR1, VL CDR2, and VL CDR3, independently; (d) the VH region comprises the amino acid sequence of SEQ ID NO: 46, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 40, with 0, 1, 2, 3, 4, 5, 6, 7, 8, 9, or 10 substitutions in each of the VL CDR1, VL CDR2, and VL CDR3, independently; (e) the VH region comprises the amino acid sequence of SEQ ID NO: 47, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 41, with 0, 1, 2, 3, 4, 5, 6, 7, 8, 9, or 10 substitutions in each of the VL CDR1, VL CDR2, and VL CDR3, independently; or (f) the VH region comprises the amino acid sequence of SEQ ID NO: 48, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 42, with 0, 1, 2, 3, 4, 5, 6, 7, 8, 9, or 10 substitutions in each of the VL CDR1, VL CDR2, and VL CDR3, independently.

In some embodiments, the VH region comprises the amino acid sequence of SEQ ID NO: 45, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 39, with 0, 1, 2, 3, 4, 5, 6, 7, 8, 9, or 10 substitutions in each of the VL CDR1, VL CDR2, and VL CDR3, independently. In some embodiments, the VH region comprises the amino acid sequence of SEQ ID NO: 43, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 37, with 0, 1, 2, 3, 4, 5, 6, 7, 8, 9, or 10 substitutions in each of the VL CDR1, VL CDR2, and VL CDR3, independently. In some embodiments, the VH region comprises the amino acid sequence of SEQ ID NO: 44, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 38, with 0, 1, 2, 3, 4, 5, 6, 7, 8, 9, or 10 substitutions in each of the VL CDR1, VL CDR2, and VL CDR3, independently. In some embodiments, the VH region comprises the amino acid sequence of SEQ ID NO: 46, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 40, with 0, 1, 2, 3, 4, 5, 6, 7, 8, 9, or 10 substitutions in each of the VL CDR1, VL CDR2, and VL CDR3, independently. In some embodiments, the VH region comprises the amino acid sequence of SEQ ID NO: 47, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 41, with 0, 1, 2, 3, 4, 5, 6, 7, 8, 9, or 10 substitutions in each of the VL CDR1, VL CDR2, and VL CDR3, independently. In some embodiments, the VH region comprises the amino acid sequence of SEQ ID NO: 48, with 0, 1, 2, 3, 4, 5, 6, 7, 8, 9, or 10 substitutions in each of the VH CDR1, VH CDR2, and VH CDR3, independently, and the VL region comprises the amino acid sequence of SEQ ID NO: 42, with 0, 1, 2, 3, 4, 5, 6, 7, 8, 9, or 10 substitutions in each of the VL CDR1, VL CDR2, and VL CDR3, independently.

In some embodiments, (a) the VH region comprises the amino acid sequence of SEQ ID NO: 45, and the VL region comprises the amino acid sequence of SEQ ID NO: 39; (b) the VH region comprises the amino acid sequence of SEQ ID NO: 43, and the VL region comprises the amino acid sequence of SEQ ID NO: 37; (c) the VH region comprises the amino acid sequence of SEQ ID NO: 44, and the VL region comprises the amino acid sequence of SEQ ID NO: 38; (d) the VH region comprises the amino acid sequence of SEQ ID NO: 46, and the VL region comprises the amino acid sequence of SEQ ID NO: 40; (e) the VH region comprises the amino acid sequence of SEQ ID NO: 47, and the VL region comprises the amino acid sequence of SEQ ID NO: 41; or (f) the VH region comprises the amino acid sequence of SEQ ID NO: 48, and the VL region comprises the amino acid sequence of SEQ ID NO: 42.

In some embodiments, the VH region comprises the amino acid sequence of SEQ ID NO: 45, and the VL region comprises the amino acid sequence of SEQ ID NO: 39. In some embodiments, the VH region comprises the amino acid sequence of SEQ ID NO: 43, and the VL region comprises the amino acid sequence of SEQ ID NO: 37. In some embodiments, the VH region comprises the amino acid sequence of SEQ ID NO: 44, and the VL region comprises the amino acid sequence of SEQ ID NO: 38. In some embodiments, the VH region comprises the amino acid sequence of SEQ ID NO: 46, and the VL region comprises the amino acid sequence of SEQ ID NO: 40. In some embodiments, the VH region comprises the amino acid sequence of SEQ ID NO: 47, and the VL region comprises the amino acid sequence of SEQ ID NO: 41. In some embodiments, the VH region comprises the amino acid sequence of SEQ ID NO: 48, and the VL region comprises the amino acid sequence of SEQ ID NO: 42.

In some embodiments, the antigen-binding protein comprises the amino acid sequence of any one of SEQ ID NOs: 25, 27, 29, 32, 34, and 36, or a sequence having at least 90%, 93%, 95%, 96%, 97%, 98%, or 99% identity to any one of SEQ ID NOs: 25, 27, 29, 32, 34, and 36. In some embodiments, the antigen-binding protein comprises the amino acid sequence of SEQ ID NO: 25. In some embodiments, the antigen-binding protein comprises the amino acid sequence of SEQ ID NO: 27.

In some embodiments, the antigen-binding protein comprises an amino acid sequence of SEQ ID NO: 25 or SEQ ID NO: 27 and the costimulatory domain comprises the amino acid sequence of SEQ ID NO: 99. In some embodiments, the antigen-binding protein comprises an amino acid sequence of SEQ ID NO: 25 and the costimulatory domain comprises the amino acid sequence of SEQ ID NO: 99. In some embodiments, the antigen-binding protein comprises an amino acid sequence of SEQ ID NO: 27 and the costimulatory domain comprises the amino acid sequence of SEQ ID NO: 99.

In some embodiments, the antigen-binding protein comprises an amino acid sequence of SEQ ID NO: 25 or SEQ ID NO: 27 and the costimulatory domain comprises the amino acid sequence of SEQ ID NO: 101. In some embodiments, the antigen-binding protein comprises an amino acid sequence of SEQ ID NO: 25 and the costimulatory domain comprises the amino acid sequence of SEQ ID NO: 101. In some embodiments, the antigen-binding protein comprises an amino acid sequence of SEQ ID NO: 27 and the costimulatory domain comprises the amino acid sequence of SEQ ID NO: 101.

In some embodiments, the transmembrane domain comprises a CD8a or CD28 transmembrane domain. In some embodiments, the transmembrane domain comprises a CD8a transmembrane domain comprising an amino acid sequence of SEQ ID NO: 95. In some embodiments, the transmembrane domain comprises a CD8a transmembrane domain having at least 90%, 93%, 95%, 96%, 97%, 98%, or 99% identity to SEQ ID NO: 95. In some embodiments, the transmembrane domain comprises a CD28 transmembrane region comprising an amino acid sequence of SEQ ID NO: 93. In some embodiments, the transmembrane domain comprises a CD28 domain having at least 90%, 93%, 95%, 96%, 97%, 98%, or 99% identity to SEQ ID NO: 93.

In some embodiments, the anti-CD70 CAR further comprises a hinge domain between the antigen binding protein and the transmembrane domain. In some embodiments, the hinge domain is a CD8a hinge domain. In some embodiments, the hinge domain is a CD8a hinge domain comprising an amino acid sequence of SEQ ID NO: 89. In some embodiments, the hinge domain is a CD8a hinge domain comprising an amino acid sequence having at least 90%, 93%, 95%, 96%, 97%, 98%, or 99% identity to SEQ ID NO: 89.

In some embodiments, the intracellular domain further comprises an activation domain. In some embodiments, the activation domain is a CD3z activation domain comprising an amino acid sequence of SEQ ID NO: 103. In some embodiments, the hinge domain is a CD3z hinge domain comprising an amino acid sequence having at least 90%, 93%, 95%, 96%, 97%, 98%, or 99% identity to SEQ ID NO: 103.

In some embodiments, the intracellular domain comprises a CD28 costimulatory domain comprising the amino acid sequence of SEQ ID NO: 99 and a CD3z activation domain comprising the amino acid sequence of SEQ ID NO: 103. In some embodiments, the intracellular domain comprises a 41BB costimulatory domain comprising the amino acid sequence of SEQ ID NO: 101 and a CD3z activation domain comprising the amino acid sequence of SEQ ID NO: 103.

In some embodiments, the antigen-binding protein comprises a sequence of SEQ ID NO: 32 and the costimulatory domain comprises a sequence of SEQ ID NO: 101; the antigen-binding protein comprises a sequence of SEQ ID NO: 32 and the costimulatory domain comprises a sequence of SEQ ID NO: 99; the antigen-binding protein comprises a sequence of SEQ ID NO: 25 and the costimulatory domain comprises a sequence of SEQ ID NO: 101; the antigen-binding protein comprises a sequence of SEQ ID NO: 25 and the costimulatory domain comprises a sequence of SEQ ID NO: 99; the antigen-binding protein comprises a sequence of SEQ ID NO: 27 and the costimulatory domain comprises a sequence of SEQ ID NO: 101; the antigen-binding protein comprises a sequence of SEQ ID NO: 27 and the costimulatory domain comprises a sequence of SEQ ID NO: 99; the antigen-binding protein comprises a sequence of SEQ ID NO: 36 and the costimulatory domain comprises a sequence of SEQ ID NO: 101; or the antigen-binding protein comprises a sequence of SEQ ID NO: 36 and the costimulatory domain comprises a sequence of SEQ ID NO: 99.

In some embodiments, the anti-CD70 CAR comprises an amino acid sequence that is at least 90% identical to the amino acid sequence of any one of SEQ ID NOs: 2, 4, 6, 8, 14, 16, 20, and 22. In some embodiments, the anti-CD70 CAR comprises an amino acid sequence that is at least 91% identical to the amino acid sequence of any one of SEQ ID NOs: 2, 4, 6, 8, 14, 16, 20, and 22. In some embodiments, the anti-CD70 CAR comprises an amino acid sequence that is at least 92% identical to the amino acid sequence of any one of SEQ ID NOs: 2, 4, 6, 8, 14, 16, 20, and 22. In some embodiments, the anti-CD70 CAR comprises an amino acid sequence that is at least 93% identical to the amino acid sequence of any one of SEQ ID NOs: 2, 4, 6, 8, 14, 16, 20, and 22. In some embodiments, the anti-CD70 CAR comprises an amino acid sequence that is at least 94% identical to the amino acid sequence of any one of SEQ ID NOs: 2, 4, 6, 8, 14, 16, 20, and 22. In some embodiments, the anti-CD70 CAR comprises an amino acid sequence that is at least 95% identical to the amino acid sequence of any one of SEQ ID NOs: 2, 4, 6, 8, 14, 16, 20, and 22. In some embodiments, the anti-CD70 CAR comprises an amino acid sequence that is at least 96% identical to the amino acid sequence of any one of SEQ ID NOs: 2, 4, 6, 8, 14, 16, 20, and 22. In some embodiments, the anti-CD70 CAR comprises an amino acid sequence that is at least 97% identical to the amino acid sequence of any one of SEQ ID NOs: 2, 4, 6, 8, 14, 16, 20, and 22. In some embodiments, the anti-CD70 CAR comprises an amino acid sequence that is at least 98% identical to the amino acid sequence of any one of SEQ ID NOs: 2, 4, 6, 8, 14, 16, 20, and 22. In some embodiments, the anti-CD70 CAR comprises an amino acid sequence that is at least 99% identical to the amino acid sequence of any one of SEQ ID NOs: 2, 4, 6, 8, 14, 16, 20, and 22. In some embodiments, the anti-CD70 CAR comprises the amino acid sequence of any one of SEQ ID NOs: 2, 4, 6, 8, 14, 16, 20, and 22.

In one embodiment, the anti-CD70 CAR comprises an amino acid sequence that is at least 90% identical to the amino acid sequence of SEQ ID NO: 2. In one embodiment, the anti-CD70 CAR comprises an amino acid sequence that is at least 91% identical to the amino acid sequence of SEQ ID NO: 2. In one embodiment, the anti-CD70 CAR comprises an amino acid sequence that is at least 92% identical to the amino acid sequence of SEQ ID NO: 2. In one embodiment, the anti-CD70 CAR comprises an amino acid sequence that is at least 93% identical to the amino acid sequence of SEQ ID NO: 2. In one embodiment, the anti-CD70 CAR comprises an amino acid sequence that is at least 94% identical to the amino acid sequence of SEQ ID NO: 2. In one embodiment, the anti-CD70 CAR comprises an amino acid sequence that is at least 95% identical to the amino acid sequence of SEQ ID NO: 2. In one embodiment, the anti-CD70 CAR comprises an amino acid sequence that is at least 96% identical to the amino acid sequence of SEQ ID NO: 2. In one embodiment, the anti-CD70 CAR comprises an amino acid sequence that is at least 97% identical to the amino acid sequence of SEQ ID NO: 2. In one embodiment, the anti-CD70 CAR comprises an amino acid sequence that is at least 98% identical to the amino acid sequence of SEQ ID NO: 2. In one embodiment, the anti-CD70 CAR comprises an amino acid sequence that is at least 99% identical to the amino acid sequence of SEQ ID NO: 2. In one embodiment, the anti-CD70 CAR comprises the amino acid sequence of SEQ ID NO: 2.

In one embodiment, the anti-CD70 CAR comprises an amino acid sequence that is at least 90% identical to the amino acid sequence of SEQ ID NO: 4. In one embodiment, the anti-CD70 CAR comprises an amino acid sequence that is at least 91% identical to the amino acid sequence of SEQ ID NO: 4. In one embodiment, the anti-CD70 CAR comprises an amino acid sequence that is at least 92% identical to the amino acid sequence of SEQ ID NO: 4. In one embodiment, the anti-CD70 CAR comprises an amino acid sequence that is at least 93% identical to the amino acid sequence of SEQ ID NO: 4. In one embodiment, the anti-CD70 CAR comprises an amino acid sequence that is at least 94% identical to the amino acid sequence of SEQ ID NO: 4. In one embodiment, the anti-CD70 CAR comprises an amino acid sequence that is at least 95% identical to the amino acid sequence of SEQ ID NO: 4. In one embodiment, the anti-CD70 CAR comprises an amino acid sequence that is at least 96% identical to the amino acid sequence of SEQ ID NO: 4. In one embodiment, the anti-CD70 CAR comprises an amino acid sequence that is at least 97% identical to the amino acid sequence of SEQ ID NO: 4. In one embodiment, the anti-CD70 CAR comprises an amino acid sequence that is at least 98% identical to the amino acid sequence of SEQ ID NO: 4. In one embodiment, the anti-CD70 CAR comprises an amino acid sequence that is at least 99% identical to the amino acid sequence of SEQ ID NO: 4. In one embodiment, the anti-CD70 CAR comprises the amino acid sequence of SEQ ID NO: 4.

In one embodiment, the anti-CD70 CAR comprises an amino acid sequence that is at least 90% identical to the amino acid sequence of SEQ ID NO: 6. In one embodiment, the anti-CD70 CAR comprises an amino acid sequence that is at least 91% identical to the amino acid sequence of SEQ ID NO: 6. In one embodiment, the anti-CD70 CAR comprises an amino acid sequence that is at least 92% identical to the amino acid sequence of SEQ ID NO: 6. In one embodiment, the anti-CD70 CAR comprises an amino acid sequence that is at least 93% identical to the amino acid sequence of SEQ ID NO: 6. In one embodiment, the anti-CD70 CAR comprises an amino acid sequence that is at least 94% identical to the amino acid sequence of SEQ ID NO: 6. In one embodiment, the anti-CD70 CAR comprises an amino acid sequence that is at least 95% identical to the amino acid sequence of SEQ ID NO: 6. In one embodiment, the anti-CD70 CAR comprises an amino acid sequence that is at least 96% identical to the amino acid sequence of SEQ ID NO: 6. In one embodiment, the anti-CD70 CAR comprises an amino acid sequence that is at least 97% identical to the amino acid sequence of SEQ ID NO: 6. In one embodiment, the anti-CD70 CAR comprises an amino acid sequence that is at least 98% identical to the amino acid sequence of SEQ ID NO: 6. In one embodiment, the anti-CD70 CAR comprises an amino acid sequence that is at least 99% identical to the amino acid sequence of SEQ ID NO: 6. In one embodiment, the anti-CD70 CAR comprises the amino acid sequence of SEQ ID NO: 6.

In one embodiment, the anti-CD70 CAR comprises an amino acid sequence that is at least 90% identical to the amino acid sequence of SEQ ID NO: 8. In one embodiment, the anti-CD70 CAR comprises an amino acid sequence that is at least 91% identical to the amino acid sequence of SEQ ID NO: 8. In one embodiment, the anti-CD70 CAR comprises an amino acid sequence that is at least 92% identical to the amino acid sequence of SEQ ID NO: 8. In one embodiment, the anti-CD70 CAR comprises an amino acid sequence that is at least 93% identical to the amino acid sequence of SEQ ID NO: 8. In one embodiment, the anti-CD70 CAR comprises an amino acid sequence that is at least 94% identical to the amino acid sequence of SEQ ID NO: 8. In one embodiment, the anti-CD70 CAR comprises an amino acid sequence that is at least 95% identical to the amino acid sequence of SEQ ID NO: 8. In one embodiment, the anti-CD70 CAR comprises an amino acid sequence that is at least 96% identical to the amino acid sequence of SEQ ID NO: 8. In one embodiment, the anti-CD70 CAR comprises an amino acid sequence that is at least 97% identical to the amino acid sequence of SEQ ID NO: 8. In one embodiment, the anti-CD70 CAR comprises an amino acid sequence that is at least 98% identical to the amino acid sequence of SEQ ID NO: 8. In one embodiment, the anti-CD70 CAR comprises an amino acid sequence that is at least 99% identical to the amino acid sequence of SEQ ID NO: 8. In one embodiment, the anti-CD70 CAR comprises the amino acid sequence of SEQ ID NO: 8.

In one embodiment, the anti-CD70 CAR comprises an amino acid sequence that is at least 90% identical to the amino acid sequence of SEQ ID NO: 14. In one embodiment, the anti-CD70 CAR comprises an amino acid sequence that is at least 91% identical to the amino acid sequence of SEQ ID NO: 14. In one embodiment, the anti-CD70 CAR comprises an amino acid sequence that is at least 92% identical to the amino acid sequence of SEQ ID NO: 14. In one embodiment, the anti-CD70 CAR comprises an amino acid sequence that is at least 93% identical to the amino acid sequence of SEQ ID NO: 14. In one embodiment, the anti-CD70 CAR comprises an amino acid sequence that is at least 94% identical to the amino acid sequence of SEQ ID NO: 14. In one embodiment, the anti-CD70 CAR comprises an amino acid sequence that is at least 95% identical to the amino acid sequence of SEQ ID NO: 14. In one embodiment, the anti-CD70 CAR comprises an amino acid sequence that is at least 96% identical to the amino acid sequence of SEQ ID NO: 14. In one embodiment, the anti-CD70 CAR comprises an amino acid sequence that is at least 97% identical to the amino acid sequence of SEQ ID NO: 14. In one embodiment, the anti-CD70 CAR comprises an amino acid sequence that is at least 98% identical to the amino acid sequence of SEQ ID NO: 14. In one embodiment, the anti-CD70 CAR comprises an amino acid sequence that is at least 99% identical to the amino acid sequence of SEQ ID NO: 14. In one embodiment, the anti-CD70 CAR comprises the amino acid sequence of SEQ ID NO: 14.

In one embodiment, the anti-CD70 CAR comprises an amino acid sequence that is at least 90% identical to the amino acid sequence of SEQ ID NO: 16. In one embodiment, the anti-CD70 CAR comprises an amino acid sequence that is at least 91% identical to the amino acid sequence of SEQ ID NO: 16. In one embodiment, the anti-CD70 CAR comprises an amino acid sequence that is at least 92% identical to the amino acid sequence of SEQ ID NO: 16. In one embodiment, the anti-CD70 CAR comprises an amino acid sequence that is at least 93% identical to the amino acid sequence of SEQ ID NO: 16. In one embodiment, the anti-CD70 CAR comprises an amino acid sequence that is at least 94% identical to the amino acid sequence of SEQ ID NO: 16. In one embodiment, the anti-CD70 CAR comprises an amino acid sequence that is at least 95% identical to the amino acid sequence of SEQ ID NO: 16. In one embodiment, the anti-CD70 CAR comprises an amino acid sequence that is at least 96% identical to the amino acid sequence of SEQ ID NO: 16. In one embodiment, the anti-CD70 CAR comprises an amino acid sequence that is at least 97% identical to the amino acid sequence of SEQ ID NO: 16. In one embodiment, the anti-CD70 CAR comprises an amino acid sequence that is at least 98% identical to the amino acid sequence of SEQ ID NO: 16. In one embodiment, the anti-CD70 CAR comprises an amino acid sequence that is at least 99% identical to the amino acid sequence of SEQ ID NO: 16. In one embodiment, the anti-CD70 CAR comprises the amino acid sequence of SEQ ID NO: 16.

In one embodiment, the anti-CD70 CAR comprises an amino acid sequence that is at least 90% identical to the amino acid sequence of SEQ ID NO: 20. In one embodiment, the anti-CD70 CAR comprises an amino acid sequence that is at least 91% identical to the amino acid sequence of SEQ ID NO: 20. In one embodiment, the anti-CD70 CAR comprises an amino acid sequence that is at least 92% identical to the amino acid sequence of SEQ ID NO: 20. In one embodiment, the anti-CD70 CAR comprises an amino acid sequence that is at least 93% identical to the amino acid sequence of SEQ ID NO: 20. In one embodiment, the anti-CD70 CAR comprises an amino acid sequence that is at least 94% identical to the amino acid sequence of SEQ ID NO: 20. In one embodiment, the anti-CD70 CAR comprises an amino acid sequence that is at least 95% identical to the amino acid sequence of SEQ ID NO: 20. In one embodiment, the anti-CD70 CAR comprises an amino acid sequence that is at least 96% identical to the amino acid sequence of SEQ ID NO: 20. In one embodiment, the anti-CD70 CAR comprises an amino acid sequence that is at least 97% identical to the amino acid sequence of SEQ ID NO: 20. In one embodiment, the anti-CD70 CAR comprises an amino acid sequence that is at least 98% identical to the amino acid sequence of SEQ ID NO: 20. In one embodiment, the anti-CD70 CAR comprises an amino acid sequence that is at least 99% identical to the amino acid sequence of SEQ ID NO: 20. In one embodiment, the anti-CD70 CAR comprises the amino acid sequence of SEQ ID NO: 20.

In one embodiment, the anti-CD70 CAR comprises an amino acid sequence that is at least 90% identical to the amino acid sequence of SEQ ID NO: 22. In one embodiment, the anti-CD70 CAR comprises an amino acid sequence that is at least 91% identical to the amino acid sequence of SEQ ID NO: 22. In one embodiment, the anti-CD70 CAR comprises an amino acid sequence that is at least 92% identical to the amino acid sequence of SEQ ID NO: 22. In one embodiment, the anti-CD70 CAR comprises an amino acid sequence that is at least 93% identical to the amino acid sequence of SEQ ID NO: 22. In one embodiment, the anti-CD70 CAR comprises an amino acid sequence that is at least 94% identical to the amino acid sequence of SEQ ID NO: 22. In one embodiment, the anti-CD70 CAR comprises an amino acid sequence that is at least 95% identical to the amino acid sequence of SEQ ID NO: 22. In one embodiment, the anti-CD70 CAR comprises an amino acid sequence that is at least 96% identical to the amino acid sequence of SEQ ID NO: 22. In one embodiment, the anti-CD70 CAR comprises an amino acid sequence that is at least 97% identical to the amino acid sequence of SEQ ID NO: 22. In one embodiment, the anti-CD70 CAR comprises an amino acid sequence that is at least 98% identical to the amino acid sequence of SEQ ID NO: 22. In one embodiment, the anti-CD70 CAR comprises an amino acid sequence that is at least 99% identical to the amino acid sequence of SEQ ID NO: 22. In one embodiment, the anti-CD70 CAR comprises the amino acid sequence of SEQ ID NO: 22.

In some embodiments, the antigen-binding protein is an scFv.

In some embodiments, the present disclosure provides for a population of cells, wherein the cell is the engineered cell of the present disclosure.

In some aspects, the cells or cell population of the current disclosure further comprise, a gRNA molecule, e.g., one or more gRNA molecules, as described herein, or a CRISPR system as described herein or comprise a genetic modification within genomic coordinates targeted by a guide RNA or CRISPR system as described herein. In an embodiment, the cell is further altered, e.g., the target sequence targeted by the gRNA molecule is further altered, e.g., to create an indel, by introduction of a gRNA molecule as described herein (or nucleic acid encoding said gRNA molecule), or a CRISPR system (or nucleic acid encoding one or more components of said CRISPR system) as described herein, e.g., altered by a method described herein. In an embodiment, the alteration results in reduced or no expression of the functional (e.g., wild type) gene product of the gene comprising the target site.

In one aspect, the cell is an animal cell. In some embodiments, the cell is a mammalian, primate, or human cell. In some embodiments, the cell is a human cell. In some embodiments, the cell is an immune effector cell (e.g., a population of immune effector cells), for example a T cell or NK cell. In embodiments, the T cell (e.g., population of T cells) is or comprises a CD4+ T cell, a CD8+ T cell, or a combination thereof. In some embodiments, the cell is a human T cell, e.g., a human T cell or a population of human T cells. In some embodiments, the cell is a population of human T cells that comprises cells that express CD4 and/or CD8. In embodiments, the cell or population of cells is autologous. In embodiments, the cell or population of cells is allogeneic.

In another aspect, the present disclosure further provides cells, such as those described above, including a second gRNA molecule as described herein, e.g., a second gRNA molecule with a guide sequence different from that of the first gRNA molecule. In other embodiments, the two or more gRNA molecules are complementary to target sites within two different genes whose gene products associate to form a molecular complex. It will be understood that in any of the aspects and embodiments of the present disclosure in which two or more target sites of different genes (or different molecular complexes, e.g., when targeting TCR and HLA-A) are targeted, that for any or all of the different gene (or molecular complex) targets, two or more gRNAs may be employed with respect to one or more of said different genes or different molecular complexes. For example, in embodiments and aspects in which expression of TCR and expression of HLA-A is reduced or eliminated, the reduced or eliminated expression of TCR may be accomplished by, for example, one gRNA targeting TRAC, or by more than one gRNA molecule targeting TRAC; while at the same time, or alternatively, targeting of HLA-A may be accomplished by, for example, one gRNA molecule targeting HLA-A or by two or more gRNA molecules targeting HLA-A. In other embodiments, the two or more, e.g. two gRNA molecules are complementary to target sites within different genes. Such cells may comprise alterations, e.g., indels, at or near each target site such that expression of the functional gene product of more than one gene is reduced or eliminated. As discussed above, in such embodiments, more than one gRNA molecule targeted to each of the different genes may be employed.

In embodiments, the cell comprises one or more gRNA molecules comprising a guide sequence complementary with a target sequence of HLA-A, HLA-B, TRAC, CIITA, TGFBR2, or CD70.

In embodiments, the present disclosure provides a cell, e.g., a cell comprising a CAR, e.g., as described herein, that comprises one or more modifications (e.g., nucleotide insertions or deletions) to an endogenous gene encoding an HLA-A, HLA-B, TRAC, CIITA, TGFBR2, and/or CD70.

In some embodiments, the engineered cell further comprises reduced or eliminated surface expression of TGFBR2 relative to an unmodified cell, the engineered cell comprising a genetic modification in the TGFBR2 gene, wherein the genetic modification comprises at least one nucleotide within the genomic coordinates: chr3: 30606864-30691614. In some embodiments, the engineered cell has reduced or eliminated surface expression of TGFBR2 relative to an unmodified cell, the engineered cell comprising genetic modification in a TGFBR2 gene, wherein the genetic modification is within the genomic coordinates: 30606891-30691605. In some embodiments, the engineered cell has reduced or eliminated surface expression of TGFBR2 relative to an unmodified cell, the engineered cell comprising a genetic modification in the TGFBR2 gene, wherein the gene modification is within the genomic coordinates chr3: 30674205-30674229. In some embodiments, the engineered cell has reduced or eliminated surface expression of TGFBR2 relative to an unmodified cell, the engineered cell comprising a genetic modification in the TGFBR2 gene, wherein the gene modification comprises at least one nucleotide within the genomic coordinates targeted by a TGFBR2 guide RNA comprising a guide sequence of SEQ ID NO: SEQ ID NO: 301]. In some embodiments, the engineered cell has reduced or eliminated surface expression of TGFBR2 relative to an unmodified cell, the engineered cell comprising genetic modification in a TGFBR2 gene, wherein the genetic modification is within the genomic coordinates: chr3: 30606864-30691614. In some embodiments, the engineered cell has reduced or eliminated surface expression of CD70 relative to an unmodified cell, the engineered cell comprising a genetic modification in the CD70 gene, wherein the genetic modification is within the genetic coordinates chr19: 6586002-6591015. In some embodiments, the genetic modification comprises an indel, a C to T substitution, or an A to G substitution within the genomic coordinates. In some embodiments, the genetic modification comprises a C to T substitution within the genomic coordinates. In some embodiments, the cell is homozygous for HLA-C. In some embodiments, the cell is homozygous for HLA-B and HLA-C.

In some embodiments, the engineered cell has reduced or eliminated surface expression of CD70 relative to an unmodified cell, the engineered cell comprising a genetic modification in the CD70 gene, wherein the gene modification comprises at least one nucleotide within the genomic coordinates targeted by a CD70 guide RNA comprising a guide sequence of SEQ ID NO: 310. In some embodiments, the engineered cell has reduced or eliminated surface expression of CD70 relative to an unmodified cell, the engineered cell comprising genetic modification in a CD70 gene, wherein the genetic modification is within the genomic coordinates: chr19: 6586028-6591018. In some embodiments, the engineered cell has reduced or eliminated surface expression of CD70 relative to an unmodified cell, the engineered cell comprising a genetic modification in the CD70 gene, wherein the gene modification comprises at least one nucleotide within the genomic coordinates chr19: 6590998-6591018. In some embodiments, the engineered cell has reduced or eliminated surface expression of CD70 relative to an unmodified cell, the engineered cell comprising a genetic modification in the CD70 gene, wherein the gene modification comprises at least one nucleotide within the genomic coordinates targeted by a CD70 guide RNA comprising a guide sequence of SEQ ID NO: 312. In some embodiments, the genetic modification comprises an indel, a C to T substitution, or an A to G substitution within the genomic coordinates. In some embodiments, the genetic modification comprises a C to T substitution within the genomic coordinates. In some embodiments, the cell is homozygous for HLA-C. In some embodiments, the cell is homozygous for HLA-B and HLA-C.

In some embodiments, the engineered cell has reduced or eliminated surface expression of TGFBR2 and CD70 relative to an unmodified cell, the engineered cell comprising a genetic modification in the TGFB2 and CD70 gene. In some embodiments, the cell is homozygous for HLA-C. In some embodiments, the cell is homozygous for HLA-B and HLA-C.

In some embodiments of the present disclosure, the engineered cell further comprises reduced or eliminated surface expression of HLA-A relative to an unmodified cell, the engineered cell comprising a genetic modification in the HLA-A gene, wherein the genetic modification is within the genetic coordinates chr6: 29942540-29945459. In some embodiments, the engineered cell has reduced or eliminated surface expression of HLA-A relative to an unmodified cell, the engineered cell comprising a genetic modification in the HLA-A gene, wherein the genetic modification is within the genomic coordinates chosen from chr6: 29942891-29942915; chr6: 29942609-29942633; chr6: 29942889-29942913; chr6: 29944471-29944495; chr6: 29944266-29944290; and chr6: 29942785-29942809. In some embodiments, the engineered cell has reduced or eliminated surface expression of HLA-A relative to an unmodified cell, the engineered cell comprising a genetic modification in the HLA-A gene, wherein the gene modification comprises at least one nucleotide within the genomic coordinates targeted by an HLA-A guide RNA comprising a guide sequence of SEQ ID NO: 403. In some embodiments, the genetic modification comprises an indel, a C to T substitution, or an A to G substitution within the genomic coordinates. In some embodiments, the genetic modification comprises a C to T substitution within the genomic coordinates. In some embodiments, the cell is homozygous for HLA-C. In some embodiments, the cell is homozygous for HLA-B and HLA-C.

In some embodiments, the engineered cell further has reduced or eliminated surface expression of HLA-B relative to an unmodified cell, the engineered cell comprising a genetic modification in the HLA-B gene, wherein the genetic modification comprises at least one nucleotide within the genetic coordinates chr6: 31354480-31357174. In some embodiments, the engineered cell has reduced or eliminated surface expression of HLA-B relative to an unmodified cell, the engineered cell comprising a genetic modification in the HLA-B gene, wherein the genetic modification is within the genomic coordinates: chr6: 31355222-31355246, chr6: 31355221-31355245, or chr6: 31355205-31355229. In some embodiments, the engineered cell has reduced or eliminated surface expression of HLA-B relative to an unmodified cell, the engineered cell comprising a genetic modification in the HLA-B gene, wherein the gene modification comprises at least one nucleotide within the genomic coordinates targeted by an HLA-B guide RNA comprising a guide sequence of SEQ ID NO: 405-407. In some embodiments, the genetic modification comprises an indel, a C to T substitution, or an A to G substitution within the genomic coordinates. In some embodiments, the genetic modification comprises a C to T substitution within the genomic coordinates. In some embodiments, the cell is homozygous for HLA-C.

In some embodiments, the engineered cell further has reduced or eliminated surface expression of TRAC relative to an unmodified cell, the engineered cell comprising a genetic modification in the TRAC gene, wherein the genetic modification comprises at least one nucleotide within the genetic coordinates chr14: 22547462-22551621. In some embodiments, the engineered cell has reduced or eliminated surface expression of TRAC relative to an unmodified cell, the engineered cell comprising a genetic modification in the TRAC gene, wherein the genetic modification is within the genomic coordinates chr14: 22547524-22547544. In some embodiments, the engineered cell has reduced or eliminated surface expression of TRAC relative to an unmodified cell, the engineered cell comprising a genetic modification in the TRAC gene, wherein the gene modification comprises at least one nucleotide within the genomic coordinates targeted by a TRAC guide RNA comprising a guide sequence of SEQ ID NO: 413. In some embodiments, the genetic modification comprises an indel, a C to T substitution, or an A to G substitution within the genomic coordinates. In some embodiments, the genetic modification comprises a C to T substitution within the genomic coordinates. In some embodiments, the cell is homozygous for HLA-C. In some embodiments, the cell is homozygous for HLA-B and HLA-C.

In some embodiments, the engineered cell further has reduced or eliminated surface expression of MHC class II relative to an unmodified cell, the engineered cell comprising a genetic modification in the CIITA gene, wherein the genetic modification is within the genetic coordinates chosen from: (a) chr16: 10877363-10907788 and (b) chr16: 10906515-10908136. In some embodiments, the engineered cell has reduced or eliminated surface expression of MHC class II relative to an unmodified cell, the engineered cell comprising a genetic modification in the CIITA gene, wherein the gene modification comprises at least one nucleotide within the genomic coordinates targeted by a CIITA guide RNA comprising a guide sequence of SEQ ID NO: 401. In some embodiments, the genetic modification comprises an indel, a C to T substitution, or an A to G substitution within the genomic coordinates. In some embodiments, the genetic modification comprises a C to T substitution within the genomic coordinates. In some embodiments, the cell is homozygous for HLA-C. In some embodiments, the cell is homozygous for HLA-B and HLA-C.

In some embodiments, the engineered cell has reduced or eliminated surface expression of HLA-A, TRAC, and MHC class II relative to an unmodified cell, the engineered cell comprising a genetic modification in the HLA-A, TRAC, and CIITA gene. In some embodiments, the cell is homozygous for HLA-C. In some embodiments, the cell is homozygous for HLA-B and HLA-C.

In some embodiments, the engineered cell has reduced or eliminated surface expression of HLA-A, HLA-B, TRAC, and MHC class II relative to an unmodified cell, the engineered cell comprising a genetic modification in the HLA-A, HLA-B, TRAC, and CIITA gene. In some embodiments, the cell is homozygous for HLA-C.

In some embodiments, the engineered cell has reduced or eliminated surface expression of HLA-A, TRAC, MHC class II, and TGFBR2 relative to an unmodified cell, the engineered cell comprising a genetic modification in the HLA-A, TRAC, CIITA, and TGFBR2 genes. In some embodiments, the cell is homozygous for HLA-C. In some embodiments, the cell is homozygous for HLA-B and HLA-C.

In some embodiments, the engineered cell has reduced or eliminated surface expression of HLA-A, HLA-B, TRAC, MHC class II, and TGFBR2 relative to an unmodified cell, the engineered cell comprising a genetic modification in the HLA-A, HLA-B, TRAC, CIITA, and TGFBR2 genes.

In some embodiments, the cell is homozygous for HLA-C.

In some embodiments, the engineered cell has reduced or eliminated surface expression of HLA-A, TRAC, MHC class II, and CD70 relative to an unmodified cell, the engineered cell comprising a genetic modification in the HLA-A, TRAC, CIITA, and CD70 genes. In some embodiments, the cell is homozygous for HLA-C. In some embodiments, the cell is homozygous for HLA-B and HLA-C.

In some embodiments, the engineered cell has reduced or eliminated surface expression of HLA-A, HLA-B, TRAC, MHC class II, and CD70 relative to an unmodified cell, the engineered cell comprising a genetic modification in the HLA-A, HLA-B, TRAC, CIITA, and CD70 genes. In some embodiments, the cell is homozygous for HLA-C.

In some embodiments, the engineered cell has reduced or eliminated surface expression of HLA-A, TRAC, MHC class II, TGFBR2, and CD70 relative to an unmodified cell, the engineered cell comprising a genetic modification in the HLA-A, TRAC, CIITA, TGFBR2, and CD70 genes. In some embodiments, the cell is homozygous for HLA-C. In some embodiments, the cell is homozygous for HLA-B and HLA-C.

In some embodiments, the engineered cell has reduced or eliminated surface expression of HLA-A, HLA-B, TRAC, MHC class II, TGFBR2, and CD70 relative to an unmodified cell, the engineered cell comprising a genetic modification in the HLA-A, TRAC, CIITA, and CD70 genes. In some embodiments, the cell is homozygous for HLA-C.

In some embodiments, the present disclosure provides for an engineered cell comprising a modified HLA-A gene, a modified TRAC gene, a modified CIITA gene, a modified TGFBR2 gene, and/or a modified CD70 gene, wherein the engineered cell expresses an anti-CD70 chimeric antigen receptor (CAR) or comprises a nucleic acid encoding an anti-CD70 CAR. In some embodiments, the cell is homozygous for HLA-C. In some embodiments, the cell is homozygous for HLA-B and HLA-C.

In some embodiments, the genetic modification in TGFBR2 comprises at least one nucleotide within the genomic coordinates: chr3: 30606864-30691614. In some embodiments, the genetic modification in TGFBR2 is within the genomic coordinates: 30606891-30691605. In some embodiments, the genetic modification in TGFBR2 is within the genomic coordinates chr3: 30674205-30674229. In some embodiments, the genetic modification in TGFBR2 comprises at least one nucleotide within the genomic coordinates targeted by a TGFBR2 guide RNA comprising a guide sequence of SEQ ID NO: 301. In some embodiments, the genetic modification in TGFBR2 is within the genomic coordinates: chr3: 30606864-30691614. In some embodiments, the genetic modification in TGFBR2 comprises at least one nucleotide within the genomic coordinates targeted by a TGFBR2 guide RNA comprising a guide sequence of SEQ ID NO: 302. In some embodiments, the genetic modification comprises an indel, a C to T substitution, or an A to G substitution within the genomic coordinates. In some embodiments, the genetic modification comprises a C to T substitution within the genomic coordinates.

In some embodiments, the genetic modification in CD70 is within the genetic coordinates chr19: 6586002-6591015. In some embodiments, the genetic modification in CD70 comprises at least one nucleotide within the genomic coordinates targeted by a CD70 guide RNA comprising a guide sequence of SEQ ID NO: 310. In some embodiments, the genetic modification in CD70 is within the genomic coordinates: chr19: 6586028-6591018. In some embodiments, the genetic modification in CD70 comprises at least one nucleotide within the genomic coordinates chr19: 6590998-6591018. In some embodiments, the genetic modification in CD70 comprises at least one nucleotide within the genomic coordinates targeted by a CD70 guide RNA comprising a guide sequence of SEQ ID NO: 312. In some embodiments, the genetic modification comprises an indel, a C to T substitution, or an A to G substitution within the genomic coordinates. In some embodiments, the genetic modification comprises a C to T substitution within the genomic coordinates.

In some embodiments, the genetic modification in HLA-A is within the genetic coordinates chr6: 29942540-29945459. In some embodiments, the genetic modification in HLA-A is within the genomic coordinates chosen from chr6: 29942891-29942915; chr6: 29942609-29942633; chr6: 29942889-29942913; chr6: 29944471-29944495; chr6: 29944266-29944290; and chr6: 29942785-29942809. In some embodiments, the genetic modification in HLA-A comprises at least one nucleotide within the genomic coordinates targeted by an HLA-A guide RNA comprising a guide sequence of SEQ ID NO: 403. In some embodiments, the genetic modification comprises an indel, a C to T substitution, or an A to G substitution within the genomic coordinates. In some embodiments, the genetic modification comprises a C to T substitution within the genomic coordinates.

In some embodiments, the genetic modification in TRAC comprises at least one nucleotide within the genetic coordinates chr14: 22547462-22551621. In some embodiments, the engineered cell has reduced or eliminated surface expression of TRAC relative to an unmodified cell, the engineered cell comprising a genetic modification in the TRAC gene, wherein the genetic modification is within the genomic coordinates chr14: 22547524-22547544. In some embodiments, the genetic modification in TRAC comprises at least one nucleotide within the genomic coordinates targeted by a TRAC guide RNA comprising a guide sequence of SEQ ID NO: 413. In some embodiments, the genetic modification comprises an indel, a C to T substitution, or an A to G substitution within the genomic coordinates. In some embodiments, the genetic modification comprises a C to T substitution within the genomic coordinates.

In some embodiments, the genetic modification in CIITA is within the genetic coordinates chosen from: (a) chr16: 10877363-10907788 and (b) chr16: 10906515-10908136. In some embodiments, the engineered cell has reduced or eliminated surface expression of CIITA relative to an unmodified cell, the engineered cell comprising a genetic modification in the CIITA gene, wherein the genetic modification is within the genomic coordinates chosen from chr16: 10906643-10906667 and chr16: 10907504-10907528. In some embodiments, the genetic modification in CIITA comprises at least one nucleotide within the genomic coordinates targeted by a CIITA guide RNA comprising a guide sequence of SEQ ID NO: 402 or 401. In some embodiments, the genetic modification comprises an indel, a C to T substitution, or an A to G substitution within the genomic coordinates. In some embodiments, the genetic modification comprises a C to T substitution within the genomic coordinates.

In some embodiments, the present disclosure provides for an engineered cell comprising a modified HLA-A gene, a modified HLA-B gene, a modified TRAC gene, a modified CIITA gene, a modified TGFBR2 gene, and/or a modified CD70 gene, wherein the engineered cell expresses an anti-CD70 chimeric antigen receptor (CAR) or comprises a nucleic acid encoding an anti-CD70 CAR. In some embodiments, the cell is homozygous for HLA-C.

In some embodiments, the genetic modification in TGFBR2 comprises at least one nucleotide within the genomic coordinates: chr3: 30606864-30691614. In some embodiments, the genetic modification in TGFBR2 is within the genomic coordinates: 30606891-30691605. In some embodiments, the genetic modification in TGFBR2 is within the genomic coordinates chr3: 30674205-30674229. In some embodiments, the genetic modification in TGFBR2 comprises at least one nucleotide within the genomic coordinates targeted by a TGFBR2 guide RNA comprising a guide sequence of SEQ ID NO: 301]. In some embodiments, the genetic modification in TGFBR2 is within the genomic coordinates: chr3: 30606864-30691614. In some embodiments, the genetic modification in TGFBR2 comprises at least one nucleotide within the genomic coordinates targeted by a TGFBR2 guide RNA comprising a guide sequence of SEQ ID NO: 302. In some embodiments, the genetic modification comprises an indel, a C to T substitution, or an A to G substitution within the genomic coordinates. In some embodiments, the genetic modification comprises a C to T substitution within the genomic coordinates.

In some embodiments, the genetic modification in CD70 is within the genetic coordinates chr19: 6586002-6591015. In some embodiments, the genetic modification in CD70 comprises at least one nucleotide within the genomic coordinates targeted by a CD70 guide RNA comprising a guide sequence of SEQ ID NO: 310. In some embodiments, the genetic modification in CD70 is within the genomic coordinates: chr19: 6586028-6591018. In some embodiments, the genetic modification in CD70 comprises at least one nucleotide within the genomic coordinates chr19: 6590998-6591018. In some embodiments, the genetic modification in CD70 comprises at least one nucleotide within the genomic coordinates targeted by a CD70 guide RNA comprising a guide sequence of SEQ ID NO: 312. In some embodiments, the genetic modification comprises an indel, a C to T substitution, or an A to G substitution within the genomic coordinates. In some embodiments, the genetic modification comprises a C to T substitution within the genomic coordinates.

In some embodiments, the genetic modification in HLA-A is within the genetic coordinates chr6: 29942540-29945459. In some embodiments, the genetic modification in HLA-A is within the genomic coordinates chosen from chr6: 29942891-29942915; chr6: 29942609-29942633; chr6: 29942889-29942913; chr6: 29944471-29944495; chr6: 29944266-29944290; and chr6: 29942785-29942809. In some embodiments, the genetic modification in HLA-A comprises at least one nucleotide within the genomic coordinates targeted by an HLA-A guide RNA comprising a guide sequence of SEQ ID NO: 403. In some embodiments, the genetic modification comprises an indel, a C to T substitution, or an A to G substitution within the genomic coordinates. In some embodiments, the genetic modification comprises a C to T substitution within the genomic coordinates.

In some embodiments, the genetic modification in HLA-B comprises at least one nucleotide within the genetic coordinates chr6: 31354480-31357174. In some embodiments, the genetic modification in HLA-B is within the genomic coordinates: chr6: 31355222-31355246, chr6: 31355221-31355245, or chr6: 31355205-31355229. In some embodiments, the genetic modification in HLA-B comprises at least one nucleotide within the genomic coordinates targeted by an HLA-B guide RNA comprising a guide sequence of SEQ ID NO: 405-407. In some embodiments, the genetic modification comprises an indel, a C to T substitution, or an A to G substitution within the genomic coordinates. In some embodiments, the genetic modification comprises a C to T substitution within the genomic coordinates.

In some embodiments, the genetic modification in TRAC comprises at least one nucleotide within the genetic coordinates chr14: 22547462-22551621. In some embodiments, the engineered cell has reduced or eliminated surface expression of TRAC relative to an unmodified cell, the engineered cell comprising a genetic modification in the TRAC gene, wherein the genetic modification is within the genomic coordinates chr14: 22547524-22547544. In some embodiments, the genetic modification in TRAC comprises at least one nucleotide within the genomic coordinates targeted by a TRAC guide RNA comprising a guide sequence of SEQ ID NO: 413. In some embodiments, the genetic modification comprises an indel, a C to T substitution, or an A to G substitution within the genomic coordinates. In some embodiments, the genetic modification comprises a C to T substitution within the genomic coordinates.

In some embodiments, the genetic modification in CIITA is within the genetic coordinates chosen from: (a) chr16: 10877363-10907788 and (b) chr16: 10906515-10908136. In some embodiments, the engineered cell has reduced or eliminated surface expression of CIITA relative to an unmodified cell, the engineered cell comprising a genetic modification in the CIITA gene, wherein the genetic modification is within the genomic coordinates chosen from chr16: 10906643-10906667 and chr16: 10907504-10907528. In some embodiments, the genetic modification in CIITA comprises at least one nucleotide within the genomic coordinates targeted by a CIITA guide RNA comprising a guide sequence of SEQ ID NO: 402. In some embodiments, the genetic modification in CIITA comprises at least one nucleotide within the genomic coordinates targeted by a CIITA guide RNA comprising a guide sequence of SEQ ID NO: 401. In some embodiments, the genetic modification comprises an indel, a C to T substitution, or an A to G substitution within the genomic coordinates. In some embodiments, the genetic modification comprises a C to T substitution within the genomic coordinates.

In some embodiments, of the present disclosure, the genetic modification comprises at least 2, at least 3, at least 4, at least 5, at least 6, at least 7, at least 8, at least 9, or at least 10 contiguous nucleotides within the genomic coordinates. In some embodiments, the genetic modification comprises an indel. In some embodiments, the genetic modification comprises an insertion of a heterologous coding sequence. In some embodiments, the genetic modification comprises at least one A to G substitution within the genomic coordinates. In some embodiments, the genetic modification comprises at least one C to T substitution within the genomic coordinates.

In some embodiments, the cell has reduced expression of TRAC protein on the surface of the cell. In some embodiments, the cell has a genetic modification in the CIITA gene. In some embodiments, the cell has reduced expression of MHC class II molecules on the surface of the cell.

In some embodiments, the present disclosure provides for a population of cells, comprising the engineered cells of the present disclosure.

In some embodiments, the present disclosure provides a pharmaceutical composition comprising the engineered cell of the disclosure.

In some aspects, the present disclosure provides for an engineered cell, population of cells, pharmaceutical composition, or method, wherein the engineered cell is an immune cell.

In some aspects, the present disclosure provides for an engineered cell, population of cells, pharmaceutical composition, or method, wherein the engineered cell is a stem cell.

In some aspects, the present disclosure provides for an engineered cell, population of cells, pharmaceutical composition, or method, wherein the engineered cell is a primary cell.

In some aspects, the present disclosure provides for an engineered cell, population of cells, pharmaceutical composition, or method, wherein the engineered cell is engineered with a genomic editing system. In some embodiments, the genomic editing system comprises an RNA-guided DNA-binding agent or a nucleic acid encoding an RNA-guided DNA-binding agent. In some embodiments, wherein the RNA-guided DNA-binding agent or the RNA-guided DNA-binding agent encoded by the nucleic acid is S. pyogenes Cas9 (SpyCas9). In some embodiments, wherein the RNA-guided DNA-binding agent or the RNA-guided DNA-binding agent encoded by the nucleic acid is N. meningitidis Cas9 (NmeCas9). In some embodiments, the RNA-guided DNA-binding agent or the RNA-guided DNA-binding agent encoded by the nucleic acid has double-stranded endonuclease activity. The RNA-guided DNA-binding agent or the RNA-guided DNA-binding agent encoded by the nucleic acid has nickase activity. In some embodiments, the RNA-guided DNA-binding agent or the RNA-guided DNA-binding agent encoded by the nucleic acid comprises a dCas9 DNA-binding domain. In some embodiments, the RNA-guided DNA-binding agent or nucleic acid encoding the RNA-guided DNA-binding agent is a A to G base editor. In some embodiments, the RNA-guided DNA-binding agent or nucleic acid encoding the RNA-guided DNA-binding agent is a C to T base editor.

In some embodiments, the guide RNA is provided to the cell in a vector. In some embodiments, the RNA-guided DNA-binding agent is provided to the cell in a vector, optionally in the same vector as the guide RNA. In some embodiments, the exogenous nucleic acid is provided to the cell in a vector. In some embodiments, the vector is a viral vector. In some embodiments, the vector is a non-viral vector.

In some embodiments, the guide RNA is provided to the cell in a lipid nanoparticle (LNP), optionally in the same LNP an RNA-guided DNA-binding agent is provided. In some embodiments, the exogenous nucleic acid is provided to the cell in a lipid nanoparticle (LNP). In some embodiments, the guide RNA is a single guide RNA. In some embodiments, the guide RNA comprises a 5′ end modification or a 3′ end modification.

In embodiments, said modifications reduce or eliminate expression of said gene. In embodiments, the present disclosure provides a cell, e.g., a cell comprising a CAR, e.g., as described herein, that is HLA-A-(e.g., has a level of expression of HLA-A greater than 50%, 60%, 70%, 80%, 90%, 95%, 96%, 97%, 98%, or 99% lower than that of an unmodified cell of the same type, as detected by FACS, e.g., FACS using an anti-HLA-A antibody), HLA-B-(e.g., has a level of expression of HLA-B greater than 50%, 60%, 70%, 80%, 90%, 95%, 96%, 97%, 98%, or 99% lower than that of an unmodified cell of the same type, as detected by FACS, e.g., FACS using an anti-HLA-B antibody), TCR-(e.g., has a level of expression of TCR greater than 50%, 60%, 70%, 80%, 90%, 95%, 96%, 97%, 98%, or 99% lower than that of an unmodified cell of the same type, as detected by FACS, e.g., FACS using an anti-CD3 antibody), CIITA-(e.g., has a level of expression of CIITA and/or a MHC class II protein greater than 50%, 60%, 70%, 80%, 90%, 95%, 96%, 97%, 98%, or 99% lower than that of an unmodified cell of the same type, as detected by FACS, e.g., FACS using an anti-CIITA antibody), TGFBR2-(e.g., has a level of expression of TGFBR2 greater than 50%, 60%, 70%, 80%, 90%, 95%, 96%, 97%, 98%, or 99% lower than that of an unmodified cell of the same type, as detected by FACS, e.g., FACS using an anti-TGFBR2 antibody), and/or CD70-(e.g., has a level of expression of CD70 greater than 50%, 60%, 70%, 80%, 90%, 95%, 96%, 97%, 98%, or 99% lower than that of an unmodified cell of the same type, as detected by FACS, e.g., FACS using an anti-CD70 antibody).

In some embodiments of the present disclosure, a cell or population of cells of the disclosure comprises: (a) a nucleic acid sequence encoding a CAR, e.g., as disclosed herein; (b) an indel at or near a sequence of a gene encoding one or more of HLA-A, HLA-B, TRAC, CIITA, TGFBR2, and CD70 or its regulatory elements, e.g., an indel at or near a target sequence of a gRNA comprising a guide sequence to one or more of HLA-A, HLA-B, TRAC, CIITA, TGFBR2, and CD70, e.g., comprising a guide sequence listed in Tables 2A-2B.

In any of the embodiments and aspects, the cell may further comprise one or more CRISPR systems, e.g., as described herein, comprising the gRNA molecule(s) indicated. In some embodiments, the cell comprises one or more ribonuclear protein (RNP) complexes each comprising a Cas9 molecule, e.g., as described herein, and a gRNA molecule comprising the indicated guide sequence, e.g., as described herein. In some embodiments, including in any of the methods described herein, where gRNAs to more than one target sequence are employed, the gRNAs (and CRISPR systems comprising said gRNAs) may be introduced into the cell simultaneously. In other embodiments, including in any of the methods described herein, where gRNAs to more than one target sequence are employed, the gRNAs (and CRISPR systems comprising said gRNAs) may be introduced into the cell sequentially.

In an aspect involving any of the aforementioned embodiments or aspects, the population of cells comprises at least 20%, e.g., at least 30%, at least 40%, at least 50%, at least 60%, at least 70%, at least 80%, at least 90%, at least 95%, or at least 99%, of cells which include an indel at or near each of the target sequences targeted by each of the gRNA molecules. Said population may be obtained, for example, by utilizing high efficiency gRNA molecules (e.g., gRNA molecules which cause an indel in >85% of said cells which are exposed to said gRNA molecule), or by enriching the population for the desired cell, e.g., by selecting for the desired cell population, e.g., by affinity chromatography or cell sorting.

### Exemplary Engineered Cell Compositions

In addition to providing for the engineered cells or cell populations as described in the above subsection, the present disclosure also provides for engineered cells, cell populations, pharmaceutical compositions, and uses thereof as follows.

In some embodiments, provided herein is an engineered human T cell comprising multiple genetic modifications and an anti-CD70 chimeric antigen receptor (CAR), wherein (a) the engineered human T cell comprises a genetic modification in the HLA-A gene and reduced or eliminated surface expression of HLA-A relative to an unmodified cell, a genetic modification in the HLA-B gene and reduced or eliminated surface expression of HLA-B relative to an unmodified cell, a genetic modification in the CIITA gene and reduced or eliminated surface expression of MHC class II relative to an unmodified cell, a genetic modification in the TGFBR2 gene and reduced or eliminated surface expression of TGFBR2 relative to an unmodified cell, and a genetic modification in the CD70 gene and reduced or eliminated surface expression of CD70 relative to an unmodified cell, and (b) the anti-CD70 CAR comprises an antigen-binding protein, or a fragment thereof, that binds to CD70, wherein the antigen-binding protein comprises a heavy chain variable (VH) region comprising a complementarity determining region 1 (VH CDR1) comprising the amino acid sequence of SEQ ID NO: 73, a VH CDR2 comprising the amino acid sequence of SEQ ID NO: 74, and a VH CDR3 comprising the amino acid sequence of SEQ ID NO: 75, and comprises a light chain variable (VL) region comprising a complementarity determining region 1 (VL CDR1) comprising the amino acid sequence of SEQ ID NO: 55, a VL CDR2 comprising the amino acid sequence of SEQ ID NO: 56, and a VL CDR3 comprising the amino acid sequence of SEQ ID NO: 57, wherein the anti-CD70 CAR is inserted into the TRAC locus.

In some embodiments, provided herein is an engineered human T cell comprising multiple genetic modifications and an anti-CD70 chimeric antigen receptor (CAR), wherein (a) the engineered human T cell comprises a genetic modification in the HLA-A gene within the genomic coordinates chr6: 29942891-29942915, a genetic modification in the HLA-B gene within the genomic coordinates chr6: 31355222-31355246, a genetic modification in the CIITA gene within the genomic coordinates chr16: 10906643-10906667, a genetic modification in the TGFBR2 gene within the genomic coordinates chr3: 30674205-30674229, and a genetic modification in the CD70 gene within the genomic coordinates chr19: 6590121-6590145, and (b) the anti-CD70 CAR comprises an antigen-binding protein, or a fragment thereof, that binds to CD70, wherein the antigen-binding protein comprises a heavy chain variable (VH) region comprising a complementarity determining region 1 (VH CDR1) comprising the amino acid sequence of SEQ ID NO: 73, a VH CDR2 comprising the amino acid sequence of SEQ ID NO: 74, and a VH CDR3 comprising the amino acid sequence of SEQ ID NO: 75, and comprises a light chain variable (VL) region comprising a complementarity determining region 1 (VL CDR1) comprising the amino acid sequence of SEQ ID NO: 55, a VL CDR2 comprising the amino acid sequence of SEQ ID NO: 56, and a VL CDR3 comprising the amino acid sequence of SEQ ID NO: 57, wherein the anti-CD70 CAR is inserted into the TRAC locus.

In some embodiments, provided herein is an engineered human T cell comprising multiple genetic modifications and an anti-CD70 chimeric antigen receptor (CAR), wherein (a) the engineered human T cell comprises a genetic modification in the HLA-A gene within the genomic coordinates chr6: 29942891-29942915, a genetic modification in the HLA-B gene within the genomic coordinates chr6: 31355222-31355246, a genetic modification in the CIITA gene within the genomic coordinates chr16: 10906643-10906667, a genetic modification in the TGFBR2 gene within the genomic coordinates chr3: 30674205-30674229, and a genetic modification in the CD70 gene within the genomic coordinates chr19: 6590121-6590145, and (b) the anti-CD70 CAR comprises an antigen-binding protein, or a fragment thereof, that binds to CD70, wherein the antigen-binding protein comprises a heavy chain variable (VH) region comprising a complementarity determining region 1 (VH CDR1) comprising the amino acid sequence of SEQ ID NO: 73, a VH CDR2 comprising the amino acid sequence of SEQ ID NO: 74, and a VH CDR3 comprising the amino acid sequence of SEQ ID NO: 75, and comprises a light chain variable (VL) region comprising a complementarity determining region 1 (VL CDR1) comprising the amino acid sequence of SEQ ID NO: 55, a VL CDR2 comprising the amino acid sequence of SEQ ID NO: 56, and a VL CDR3 comprising the amino acid sequence of SEQ ID NO: 57, wherein the anti-CD70 CAR is inserted into the TRAC locus within the genomic coordinates chr14: 22547524-22547544.

In some embodiments, provided herein is a pharmaceutical composition comprising a population of T cells, wherein the population of T cells comprises CD4+ T cells and/or CD8+ T cells, which comprise an engineered human T cell comprising multiple genetic modifications and an anti-CD70 chimeric antigen receptor (CAR), wherein (a) the engineered human T cell comprises a genetic modification in the HLA-A gene and reduced or eliminated surface expression of HLA-A relative to an unmodified cell, a genetic modification in the HLA-B gene and reduced or eliminated surface expression of HLA-B relative to an unmodified cell, a genetic modification in the CIITA gene and reduced or eliminated surface expression of MHC class II relative to an unmodified cell, a genetic modification in the TGFBR2 gene and reduced or eliminated surface expression of TGFBR2 relative to an unmodified cell, and a genetic modification in the CD70 gene and reduced or eliminated surface expression of CD70 relative to an unmodified cell, and (b) the anti-CD70 CAR comprises an antigen-binding protein, or a fragment thereof, that binds to CD70, wherein the antigen-binding protein comprises a heavy chain variable (VH) region comprising a complementarity determining region 1 (VH CDR1) comprising the amino acid sequence of SEQ ID NO: 73, a VH CDR2 comprising the amino acid sequence of SEQ ID NO: 74, and a VH CDR3 comprising the amino acid sequence of SEQ ID NO: 75, and comprises a light chain variable (VL) region comprising a complementarity determining region 1 (VL CDR1) comprising the amino acid sequence of SEQ ID NO: 55, a VL CDR2 comprising the amino acid sequence of SEQ ID NO: 56, and a VL CDR3 comprising the amino acid sequence of SEQ ID NO: 57, wherein the anti-CD70 CAR is inserted into the TRAC locus.

In some embodiments, provided herein is a population of T cells, wherein the population of T cells comprises CD4+ T cells and/or CD8+ T cells, which comprise an engineered human T cell comprising multiple genetic modifications and an anti-CD70 chimeric antigen receptor (CAR), wherein (a) the engineered human T cell comprises a genetic modification in the HLA-A gene within the genomic coordinates chr6: 29942891-29942915, a genetic modification in the HLA-B gene within the genomic coordinates chr6: 31355222-31355246, a genetic modification in the CIITA gene within the genomic coordinates chr16: 10906643-10906667, a genetic modification in the TGFBR2 gene within the genomic coordinates chr3: 30674205-30674229, and a genetic modification in the CD70 gene within the genomic coordinates chr19: 6590121-6590145, and (b) the anti-CD70 CAR comprises an antigen-binding protein, or a fragment thereof, that binds to CD70, wherein the antigen-binding protein comprises a heavy chain variable (VH) region comprising a complementarity determining region 1 (VH CDR1) comprising the amino acid sequence of SEQ ID NO: 73, a VH CDR2 comprising the amino acid sequence of SEQ ID NO: 74, and a VH CDR3 comprising the amino acid sequence of SEQ ID NO: 75, and comprises a light chain variable (VL) region comprising a complementarity determining region 1 (VL CDR1) comprising the amino acid sequence of SEQ ID NO: 55, a VL CDR2 comprising the amino acid sequence of SEQ ID NO: 56, and a VL CDR3 comprising the amino acid sequence of SEQ ID NO: 57, wherein the anti-CD70 CAR is inserted into the TRAC locus.

In some embodiments, provided herein is a population of T cells, wherein the population of T cells comprises CD4+ T cells and/or CD8+ T cells, which comprise an engineered human T cell comprising multiple genetic modifications and an anti-CD70 chimeric antigen receptor (CAR), wherein (a) the engineered human T cell comprises a genetic modification in the HLA-A gene within the genomic coordinates chr6: 29942891-29942915, a genetic modification in the HLA-B gene within the genomic coordinates chr6: 31355222-31355246, a genetic modification in the CIITA gene within the genomic coordinates chr16: 10906643-10906667, a genetic modification in the TGFBR2 gene within the genomic coordinates chr3: 30674205-30674229, and a genetic modification in the CD70 gene within the genomic coordinates chr19: 6590121-6590145, and (b) the anti-CD70 CAR comprises an antigen-binding protein, or a fragment thereof, that binds to CD70, wherein the antigen-binding protein comprises a heavy chain variable (VH) region comprising a complementarity determining region 1 (VH CDR1) comprising the amino acid sequence of SEQ ID NO: 73, a VH CDR2 comprising the amino acid sequence of SEQ ID NO: 74, and a VH CDR3 comprising the amino acid sequence of SEQ ID NO: 75, and comprises a light chain variable (VL) region comprising a complementarity determining region 1 (VL CDR1) comprising the amino acid sequence of SEQ ID NO: 55, a VL CDR2 comprising the amino acid sequence of SEQ ID NO: 56, and a VL CDR3 comprising the amino acid sequence of SEQ ID NO: 57, wherein the anti-CD70 CAR is inserted into the TRAC locus within the genomic coordinates chr14: 22547524-22547544.

In some embodiments, in the engineered human T cell, the VH region comprises the amino acid sequence of SEQ ID NO: 45, and the VL region comprises the amino acid sequence of SEQ ID NO: 39. In some embodiments, in the engineered human T cell, the anti-CD70 CAR comprises the amino acid sequence of SEQ ID NO: 4.

In some embodiments, the engineered human T cell is a CD4+ or CD8+ T cell. In some embodiments, the engineered human T cell is a CD4+ T cell. In some embodiments, the engineered human T cell is a CD8+ T cell.

In some embodiments, the engineered human T cell is homozygous for HLA-C. In some embodiments, the engineered human T cell is homozygous for HLA-B and for HLA-C.

In some embodiments, provided herein is a method of administering the above engineered human T cell or pharmaceutical composition to a subject in need thereof. In some embodiments, provided herein is a method of administering the above engineered human T cell or pharmaceutical composition to a subject as an adoptive cell transfer (ACT) therapy.

In some embodiments, provided herein is a method of treating a disease or disorder, comprising administering the above engineered human T cell or pharmaceutical composition to a subject in need thereof.

In some embodiments, provided herein is the above engineered human T cell or pharmaceutical composition, for use in administration to a subject as an adoptive cell transfer (ACT) therapy. In some embodiments, provided herein is the above engineered human T cell or pharmaceutical composition, for use in treating a subject having a cancer. In some embodiments, provided herein is the above engineered human T cell or pharmaceutical composition, for use in treating a subject having an infectious disease. In some embodiments, provided herein is the above engineered human T cell or pharmaceutical composition, for use in treating a subject having an autoimmune disease.

In some embodiments, the disease or disorder is a cancer. In some embodiments, the cancer is a solid tumor or a hematological malignancy. In some embodiments, the solid tumor is renal cell carcinoma. In some embodiments, the hematological malignancy is acute myeloid leukemia. In some embodiments, the hematological malignancy is multiple myeloma.

### Methods and Compositions for Generating Cells Comprising Anti-CD70 Chimeric Antigen Receptors (CARs) and Reduced or Eliminated Surface Expression of One or More of HLA-A, HLA-B, TRAC, MHC Class II, TGFBR2, and CD70

In some embodiments, a method of making an engineered cell is provided, the method comprising contacting a cell with (a) a nucleic acid or mRNA encoding an anti-CD70 CAR, or an expression vector encoding a nucleic acid or mRNA encoding an anti-CD70 CAR; and (b) at least one genomic editing tool comprising a genomic editor and at least one guide RNA, wherein the at least one guide RNA targets a genomic locus chosen from the HLA-A, HLA-B, TRAC, CIITA, TGFBR2, and CD70 locus.

In some embodiments, a method of making an engineered cell is provided, the method comprising (a) providing an engineered cell which has reduced or eliminated surface expression of one or both of TGFBR2 and CD70 relative to an unmodified cell; and (b) contacting the cell with a nucleic acid or mRNA encoding an anti-CD70 CAR, or an expression vector encoding a nucleic acid or mRNA encoding an anti-CD70 CAR.

In some embodiments, a method of making an engineered cell is provided, the method comprising (a) providing an engineered cell which has reduced or eliminated surface expression of one or more of HLA-A, HLA-B, MHC class II, TRAC, TGFBR2, and CD70, relative to an unmodified cell; and (b) contacting the cell with the nucleic acid or mRNA encoding an anti-CD70 CAR, or an expression vector encoding a nucleic acid or mRNA encoding an anti-CD70 CAR.

### 1. Anti-CD70 CAR Knock-In

The present disclosure provides methods and compositions for generating cells comprising an anti-CD70 CAR encoded by an exogenous nucleic acid. In some embodiments, the anti-CD70 CAR is an anti-CD70 CAR disclosed herein.

In some embodiments, the methods comprise contacting a cell with an exogenous nucleic acid encoding an anti-CD70 CAR. In some embodiments, the anti-CD70 CAR is an anti-CD70 CAR disclosed herein.

In some embodiments, an exogenous nucleic acid encoding an anti-CD70 CAR is inserted into the genome of the target cell. In some embodiments, the exogenous nucleic acid is integrated into the genome of the target cell. In some embodiments, the exogenous nucleic acid is integrated into the genome of the target cell by homologous recombination (HR). In some embodiments, the exogenous nucleic acid is integrated into the genome of the target cell by blunt end insertion. In some embodiments, the exogenous nucleic acid is integrated into the genome of the target cell by non-homologous end joining. In some embodiments, the exogenous nucleic acid is integrated into a safe harbor locus in the genome of the cell. In some embodiments, the exogenous nucleic acid is integrated into one of the TRAC locus or CIITA locus. In some embodiments, the lipid nucleic acid assembly composition is a lipid nanoparticle (LNP).

In some embodiments, the methods produce a composition comprising an engineered cell comprising an exogenous nucleic acid encoding an anti-CD70 CAR.

In some embodiments, an allogeneic cell is provided wherein the cell comprises an exogenous nucleic acid encoding an anti-CD70 CAR.

The term “exogenous nucleic acid,” “template nucleic acid,” or “donor template,” as used herein, refers to a nucleic acid to be inserted at or near a target sequence that has been modified, e.g., cleaved, by a CRISPR system of the present disclosure. In an embodiment, an endogenous nucleic acid sequence at or near the target site is modified to have some or all of the sequence of the exogenous nucleic acid, typically at or near cleavage site(s). In an embodiment, the exogenous nucleic acid is single stranded. In an alternate embodiment, the exogenous nucleic acid is double stranded. In an embodiment, the template nucleic acid is DNA, e.g., double stranded DNA. In an alternate embodiment, the template nucleic acid is single stranded DNA.

In certain embodiments, the exogenous nucleic acid comprises sequence encoding an anti-CD70 CAR, e.g., an anti-CD70 CAR as described herein.

In an embodiment, the template nucleic acid alters the structure of the target position by participating in a homology directed repair event. In an embodiment, the template nucleic acid alters the sequence of the target position. In an embodiment, the template nucleic acid results in the incorporation of a modified, or non-naturally occurring base into the target nucleic acid.

In an embodiment, a single nick can be used to induce HDR. It is contemplated herein that a single nick can be used to increase the ratio of HDR, HR or NHEJ at a given cleavage site.

The double strand break or single strand break in one of the strands should be sufficiently close to target position such that correction occurs. In an embodiment, the distance is not more than 50, 100, 200, 300, 350 or 400 nucleotides. While not wishing to be bound by theory, it is believed that the break should be sufficiently close to target position such that the break is within the region that is subject to exonuclease-mediated removal during end resection. If the distance between the target position and a break is too great, the mutation may not be included in the end resection and, therefore, may not be corrected, as donor sequence may only be used to correct sequence within the end resection region.

The homology arm should extend at least as far as the region in which end resection may occur, e.g., in order to allow the resected single stranded overhang to find a complementary region within the donor template. The overall length could be limited by parameters such as plasmid size or viral packaging limits. In an embodiment, a homology arm does not extend into repeated elements, e.g., ALU repeats, LINE repeats. A template may have two homology arms of the same or different lengths.

Exemplary homology arm lengths include at least 25, 50, 100, 250, 500, 750 or 1000 nucleotides.

Target position, as used herein, refers to a site on a target nucleic acid (e.g., the chromosome) that is modified by a Cas9 molecule-dependent process. For example, the target position can be a modified Cas9 molecule cleavage of the target nucleic acid and template nucleic acid directed modification, e.g., correction, of the target position. In an embodiment, a target position can be a site between two nucleotides, e.g., adjacent nucleotides, on the target nucleic acid into which one or more nucleotides is added. The target position may comprise one or more nucleotides that are altered, e.g., corrected, by a template nucleic acid. In an embodiment, the target position is within a target sequence (e.g., the sequence to which the gRNA binds). In an embodiment, a target position is upstream or downstream of a target sequence (e.g., the sequence to which the gRNA binds).

Typically, the template sequence undergoes a breakage mediated or catalyzed recombination with the target sequence. In an embodiment, the template nucleic acid includes a sequence that corresponds to a site on the target sequence that is cleaved by a Cas9 mediated cleavage event. In an embodiment, the template nucleic acid includes sequence that corresponds to both, a first site on the target sequence that is cleaved in a first Cas9 mediated event, and a second site on the target sequence that is cleaved in a second Cas9 mediated event.

A template nucleic acid comprises the following components: [5′ homology arm]-[insertion sequence]-[3′ homology arm]. The homology arms provide for recombination into the chromosome, which can replace the undesired element, e.g., a mutation or signature, with the replacement sequence. In an embodiment, the homology arms flank the most distal cleavage sites.

In an embodiment, the 3′ end of the 5′ homology arm is the position next to the 5′ end of the replacement sequence. In an embodiment, the 5′ homology arm can extend at least 10, 20, 30, 40, 50, 60, 70, 80, 90, 100, 120, 150, 180, 200, 300, 400, 500, 600, 700, 800, 900, 1000, 1500, or 2000 nucleotides 5′ from the 5′ end of the replacement sequence.

In an embodiment, the 5′ end of the 3′ homology arm is the position next to the 3′ end of the replacement sequence. In an embodiment, the 3′ homology arm can extend at least 10, 20, 30, 40, 50, 60, 70, 80, 90, 100, 120, 150, 180, 200, 300, 400, 500, 600, 700, 800, 900, 1000, 1500, or 2000 nucleotides 3′ from the 3′ end of the replacement sequence.

It is contemplated herein that one or both homology arms may be shortened to avoid including certain sequence repeat elements, e.g., Alu repeats, LINE elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

It is contemplated herein that template nucleic acids for correcting a mutation may designed for use as a single-stranded oligonucleotide (ssODN). When using a ssODN, 5′ and 3′ homology arms may range up to about 200 base pairs (bp) in length, e.g., at least 25, 50, 75, 100, 125, 150, 175, or 200 bp in length. Longer homology arms are also contemplated for ssODNs as improvements in oligonucleotide synthesis continue to be made.

In one aspect, the insertion sequence comprises nucleic acid sequence that encodes an anti-CD70 chimeric antigen receptor, e.g., as described herein. In one embodiment the insertion sequence further comprises a promotor operably linked to the nucleic acid sequence encoding a chimeric antigen receptor, e.g., an EF-1 alpha promoter. In one aspect, the insertion sequence comprises a vector encoding a chimeric antigen receptor, e.g., as described herein, or a portion thereof.

### 2. HLA-A, HLA-B, TRAC, MHC Class II, TGFBR2, and CD70 Knock Out

In some embodiments, the anti-CD70 CAR may be transduced into a cell. In some embodiments, the methods comprise contacting a cell with a donor nucleic acid encoding the anti-CD70 CAR for insertion into the genome of the cell.

In some embodiments, multiplex gene editing may further be performed on a cell comprising an anti-CD70 CAR. In some embodiments, the methods comprise reducing or eliminating surface expression of one or more of HLA-A, HLA-B, TRAC, or MHC class II proteins comprising genetically modifying one or more of the HLA-A, HLA-B, TRAC, or CIITA genes comprising contacting the cell with a composition comprising one or more HLA-A, HLA-B, TRAC, or CIITA guide RNAs disclosed herein; and optionally an RNA-guided DNA binding agent or a nucleic acid encoding an RNA-guided DNA binding agent.

In some embodiments, the methods comprise reducing or eliminating surface expression of one or more of TGFBR2 or CD70 proteins comprising genetically modifying one or more of the TGFBR2 or CD70 genes comprising contacting the cell with a composition comprising one or more TGFBR2 or CD70 guide RNAs disclosed herein; and optionally an RNA-guided DNA binding agent or a nucleic acid encoding an RNA-guided DNA binding agent.

In some embodiments, multiplex gene editing may be performed in a cell. In some embodiments, the methods comprise reducing or eliminating surface expression of HLA-A, HLA-B, TRAC, MHC class II, TGFBR2, and CD70 protein and reducing or eliminating expression of CIITA protein, comprising genetically modifying the HLA-A, HLA-B, TRAC, CIITA, TGFBR2, and CD70 genes, comprising simultaneously contacting the cell with a first composition comprising an HLA-A guide RNA disclosed herein; and optionally a first RNA-guided DNA binding agent or a nucleic acid encoding an RNA-guided DNA binding agent; a second composition comprising an HLA-B guide RNA disclosed herein; and optionally a second RNA-guided DNA binding agent or a nucleic acid encoding an RNA-guided DNA binding agent; a third composition comprising a TRAC guide RNA disclosed herein; and optionally a third RNA-guided DNA binding agent or a nucleic acid encoding an RNA-guided DNA binding agent; a fourth composition comprising a CIITA guide RNA disclosed herein; and optionally a fourth RNA-guided DNA binding agent or a nucleic acid encoding an RNA-guided DNA binding agent; a fifth composition comprising a TGFBR2 guide RNA disclosed herein; and optionally a fifth RNA-guided DNA binding agent or a nucleic acid encoding an RNA-guided DNA binding agent; a sixth composition comprising a CD70 guide RNA disclosed herein; and optionally a sixth RNA-guided DNA binding agent or a nucleic acid encoding an RNA-guided DNA binding agent.

In some embodiments, multiplex gene editing may be performed sequentially. In some embodiments, multiplex gene editing may be performed serially.

In some embodiments, Multiplex gene editing may be performed simultaneously.

Multiplex gene editing may be performed using genome editors or genome editing components orthogonal to each other. As used herein, the term “orthogonal” refers to any two genomic editors (e.g., base editors, nucleases, nickases, or cleavases) where each is capable of recognizing its own target(s) via its cognate guide RNA(s) but is not compatible with the guide RNA(s) cognate to the other genomic editor, e.g., each is not capable of recognizing the target(s) of the other genomic editor via the guide RNA(s) cognate to the other genomic editor. For example, an N. meningitidis Cas9 (NmeCas9) nickase may be capable of recognizing a genomic locus via a guide RNA cognate to the NmeCas9 nickase, and an S. pyogenes Cas9 (SpyCas9) cleavase may be capable of recognizing another genomic locus via a guide RNA cognate to the SpyCas9 cleavase. In this example, the NmeCas9 nickase and the SpyCas9 cleavase are orthogonal to each other. Although in this example, the NmeCas9 nickase and the SpyCas9 cleavase are derived from different organisms, two genomic editors need not be derived from different organisms to be orthogonal to each other.

In some embodiments, a method of genetically modifying a cell is provided, the method comprising: (a) contacting the cell with a first genomic editing tool comprising a base editor and at least one guide RNA (gRNA) that targets at least one genomic locus and that is cognate to the base editor; and (b) contacting the cell with a second genomic editing tool comprising an RNA-guided cleavase and at least one gRNA that targets at least one genomic locus and that is cognate to the RNA-guided cleavase, wherein the base editor is orthogonal to the RNA-guided cleavase, thereby producing at least two genome edits in the cell. In some embodiments, the method disclosed herein further comprises culturing the cell, thereby producing the population of cells comprising edited cells comprising at least two edits per cell. In some embodiments, the base editor is a C to T base editor, optionally comprising a cytidine deaminase. In some embodiments, the RNA-guided DNA cleavase comprises an S. pyogenes (Spy) Cas9 cleavase and the base editor comprises an N. meningitidis (Nme) Cas9 nickase.

In further embodiments, the method disclosed herein further comprises contacting the cell with a nucleic acid encoding an anti-CD70 CAR for insertion into an editing site (e.g., a double strand break) at TRAC locus. In further embodiments, the nucleic acid encoding an anti-CD70 CAR is within an expression vector. In further embodiments, the expression vector is an AAV vector.

In some embodiments, the at least one gRNA that is cognate to the base editor targets one or more genes chosen from the TGFBR2 locus, the CD70 locus, the HLA-A locus, the HLA-B locus, and the CIITA locus. In some embodiments, the at least one gRNA that is cognate to the RNA-guided cleavase targets the TRAC locus.

In some embodiments, the at least one gRNA that is cognate to the base editor comprises one or more gRNA selected from a gRNA that targets the HLA-A locus, a gRNA that targets a gRNA that targets the HLA-B locus, a gRNA that targets a gRNA that targets the CIITA locus, a gRNA that targets the TGFBR2 locus, and a gRNA that targets the CD70 locus, and the at least one gRNA that is cognate to the RNA-guided cleavase comprises a gRNA that targets the TRAC locus.

In some embodiments, the at least one gRNA that is cognate to the base editor comprises a gRNA that targets the HLA-A locus and a gRNA that targets the CIITA locus, and the at least one gRNA that is cognate to the RNA-guided cleavase comprises a gRNA that targets the TRAC locus.

In some embodiments, the at least one gRNA that is cognate to the base editor comprises one or more gRNA selected from a gRNA that targets the HLA-A locus, a gRNA that targets a gRNA that targets the HLA-B locus, and a gRNA that targets a gRNA that targets the CIITA locus, and the at least one gRNA that is cognate to the RNA-guided cleavase comprises a gRNA that targets the TRAC locus.

In some embodiments, the at least one gRNA that is cognate to the base editor comprises a gRNA that targets the TGFBR2 locus one or more gRNA selected from a gRNA that targets the HLA-A locus, a gRNA that targets a gRNA that targets the HLA-B locus, a gRNA that targets a gRNA that targets the CIITA locus, and the at least one gRNA that is cognate to the RNA-guided cleavase comprises a gRNA that targets the TRAC locus.

In some embodiments, the at least one gRNA that is cognate to the base editor comprises a gRNA that targets the CD70 locus and one or more gRNA selected from a gRNA that targets the HLA-A locus, a gRNA that targets a gRNA that targets the HLA-B locus, a gRNA that targets a gRNA that targets the CIITA locus; and the at least one gRNA that is cognate to the RNA-guided cleavase comprises a gRNA that targets the TRAC locus.

In some embodiments, the at least one gRNA that is cognate to the base editor comprises a gRNA that targets the TGFBR2 locus, a gRNA that targets the CD70 locus one or more gRNA selected from a gRNA that targets the HLA-A locus, a gRNA that targets a gRNA that targets the HLA-B locus, a gRNA that targets a gRNA that targets the CIITA locus, and the at least one gRNA that is cognate to the RNA-guided cleavase comprises a gRNA that targets the TRAC locus.

In some embodiments, the at least one gRNA that is cognate to the base editor comprises a gRNA that targets the TGFBR2 locus and a gRNA that targets the CD70 locus, and the at least one gRNA that is cognate to the RNA-guided cleavase comprises a gRNA that targets the TRAC locus.

In some embodiments, the at least one gRNA that is cognate to the base editor comprises one or more gRNA selected from a gRNA that targets the HLA-A locus comprising a guide sequence of SEQ ID NO: 403, a gRNA that targets the HLA-B locus comprising a guide sequence of SEQ ID NO: 406, a gRNA that targets the CIITA locus comprising a guide sequence of SEQ ID NO: 402, a gRNA that targets the TGFBR2 locus comprising a guide sequence of SEQ ID NO: 301, and a gRNA that targets the CD70 locus comprising a guide sequence of SEQ ID NO: 310, and the at least one gRNA that is cognate to the RNA-guided cleavase comprises a gRNA that targets the TRAC locus comprising a guide sequence of SEQ ID NO: 413.

In some embodiments, the at least one gRNA that is cognate to the base editor comprises one or more gRNA selected from a gRNA that targets the HLA-A locus comprising a sequence at least 90%, 93%, 95%, 96%, 97%, 98%, 99% or 100% identity to SEQ ID NO: 446, a gRNA that targets the HLA-B locus comprising a sequence at least 90%, 93%, 95%, 96%, 97%, 98%, 99% or 100% identity to SEQ ID NO: 452, a gRNA that targets the CIITA locus comprising a sequence at least 90%, 93%, 95%, 96%, 97%, 98%, 99% or 100% identity to SEQ ID NO: 444, a gRNA that targets the TGFBR2 locus comprising a sequence at least 90%, 93%, 95%, 96%, 97%, 98%, 99% or 100% identity to SEQ ID NO: 342, and a gRNA that targets the CD70 locus comprising a sequence at least 90%, 93%, 95%, 96%, 97%, 98%, 99% or 100% identity to SEQ ID NO: 354, and the at least one gRNA that is cognate to the RNA-guided cleavase comprises a gRNA that targets the TRAC locus a sequence at least 90%, 93%, 95%, 96%, 97%, 98%, 99% or 100% identity to SEQ ID NO: 464.

In some embodiments, the at least one gRNA that is cognate to the base editor comprises one or more gRNA that comprises a gRNA that targets the HLA-A locus comprising a guide sequence of SEQ ID NO: 403, a gRNA that targets the HLA-B locus comprising a guide sequence of SEQ ID NO: 406, a gRNA that targets the CIITA locus comprising a guide sequence of SEQ ID NO: 402, a gRNA that targets the TGFBR2 locus comprising a guide sequence of SEQ ID NO: 301, and a gRNA that targets the CD70 locus comprising a guide sequence of SEQ ID NO: 310, and the at least one gRNA that is cognate to the RNA-guided cleavase comprises a gRNA that targets the TRAC locus comprising a guide sequence of SEQ ID NO: 413.

In some embodiments, the at least one gRNA that is cognate to the base editor comprises one or more gRNA that comprises a guide RNA comprising the sequence of SEQ ID NO: 446, a gRNA comprising the sequence of SEQ ID NO: 452, a gRNA comprising the sequence of SEQ ID NO: 444, a gRNA comprising the sequence of f SEQ ID NO: 342, and a gRNA comprising the sequence of SEQ ID NO: 354, and the at least one gRNA that is cognate to the RNA-guided cleavase comprises a gRNA comprising the sequence of SEQ ID NO: 464.

In some embodiments, the base editor, the RNA-guided cleavase, and at least one gRNA that are cognate to the RNA-guided cleavase or the base editor and target different genomic loci are contained in a same lipid nanoparticle (LNP) or different LNPs.

In some embodiments, the LNPs comprise a first group of distinct LNPs, and a second group of distinct LNPs, and optionally, a third group of distinct LNPs. In some embodiments, the first group of distinct LNPs comprises 2, 3, 4, or 5 LNPs, the second group of distinct LNPs comprises 2, 3, 4, or 5 LNPs, and the third group of distinct LNPs, when present, comprises 2, 3, 4, or 5 LNPs. In some embodiments, the first group of distinct LNPs comprises 2, 3 or 4 LNPs and the second group of distinct LNPs comprises 2, 3 or 4 LNPs.

In some embodiments, the first group of distinct LNPs, the second group of distinct LNPs, and the third group of distinct LNPs, when present, are delivered to the cell sequentially. In some embodiments, the second group of distinct LNPs is delivered to the cell 1, 2, or 3 days after the first group of distinct LNPs is delivered to the cell, and wherein the third group of distinct LNPs, when present, is delivered to the cell 1, 2, or 3 days after the second group of distinct LNPs is delivered to the cell.

In some embodiments, the method disclosed herein comprises: (a) contacting a cell with (a) a first group of distinct LNPs comprising a first LNP comprising the base editor and comprising a first gRNA that targets the HLA-A locus; a second LNP comprising the base editor and comprising a second gRNA that targets the HLA-B locus, a third LNP comprising the base editor and comprising a third gRNA that targets the CIITA locus, and a fourth lipid nanoparticle (LNP) comprising a uracil glycosylase inhibitor (UGI); (b) contacting a cell with (i) a second group of LNPs comprising a fifth LNP comprising the base editor and comprising a gRNA that targets the TGFBR2 locus; a sixth LNP comprising the base editor and comprising a gRNA that targets the CD70 locus; a seventh LNP comprising the RNA-guided DNA cleavase and a gRNA that targets the TRAC locus, and an eighth LNP comprising a UGI; and (ii) a nucleic acid encoding an anti-CD70 CAR for insertion into an editing site (e.g., a double strand break) at the TRAC locus. In some embodiments, the nucleic acid encoding an anti-CD70 CAR is provided in an LNP. In some embodiments, the nucleic acid encoding an anti-CD70 CAR is provided in an expression vector, optionally an AAV vector. In some embodiments, the step (b) is performed, 1, 2, or 3 days after the step (a).

In some embodiments, the method disclosed herein comprises: (a) contacting a cell with (a) a first group of distinct LNPs comprising a first LNP comprising the base editor and comprising a first gRNA that targets the HLA-A locus; a second LNP comprising the base editor and comprising a third gRNA that targets the CIITA locus, and a third LNP comprising a uracil glycosylase inhibitor (UGI); (b) contacting a cell with (i) a second group of LNPs comprising a fourth LNP comprising the base editor and comprising a gRNA that targets the TGFBR2 locus; a fifth LNP comprising the base editor and comprising a gRNA that targets the CD70 locus; a sixth LNP comprising the RNA-guided DNA cleavase and a gRNA that targets the TRAC locus, and a seventh LNP comprising a UGI; and (ii) a nucleic acid encoding an anti-CD70 CAR for insertion into an editing site (e.g., a double strand break) at the TRAC locus. In some embodiments, the nucleic acid encoding an anti-CD70 CAR is provided in an LNP. In some embodiments, the nucleic acid encoding an anti-CD70 CAR is provided in an expression vector, optionally an AAV vector. In some embodiments, the step (b) is performed, 1, 2, or 3 days after the step (a).

In some embodiments, the method disclosed herein comprises: (a) contacting a cell with (a) a first group of distinct LNPs comprising a first LNP comprising the base editor and comprising a first gRNA that targets the TGFBR2 locus; a second LNP comprising the base editor and comprising a second gRNA that targets the CD70 locus; a third LNP comprising the base editor and comprising a third gRNA that targets the HLA-A locus; a fourth LNP comprising the base editor and comprising a fourth gRNA that targets the HLA-B locus, a fifth LNP comprising the base editor and comprising a fifth gRNA that targets the CIITA locus, and a sixth lipid nanoparticle (LNP) comprising a uracil glycosylase inhibitor (UGI); (b) contacting a cell with (i) a seventh LNP comprising the RNA-guided DNA cleavase and a sixth gRNA that targets the TRAC locus and (ii) a nucleic acid encoding an anti-CD70 CAR for insertion into an editing site (e.g., a double strand break) at the TRAC locus. In some embodiments, the nucleic acid encoding an anti-CD70 CAR is provided in an LNP. In some embodiments, the nucleic acid encoding an anti-CD70 CAR is provided in an expression vector, optionally an AAV vector. In some embodiments, the step (b) is performed 1, 2, or 3 days after the step (a).

In some embodiments, the method disclosed herein comprises: (a) contacting a cell with (a) a first group of distinct LNPs comprising a first LNP comprising a nucleic acid encoding a uracil glycosylase inhibitor (UGI); a second LNP comprising the base editor and comprising a gRNA that targets the TGFBR2 locus; a third LNP comprising the base editor and comprising a gRNA that targets the HLA-A locus; and a fourth LNP comprising the base editor and comprising a gRNA that targets the HLA-B locus; (b) contacting a cell with (i) a second group of distinct LNPs comprising a fifth LNP comprising the base editor and comprising a gRNA that targets the CD70 locus, a six LNP comprising the base editor and comprising a gRNA that targets the CIITA locus, a seventh LNP comprising a nucleic acid encoding a UGI, an eighth LNP comprising the RNA-guided DNA cleavase and a gRNA that targets the TRAC locus and (ii) a nucleic acid encoding an anti-CD70 CAR for insertion into an editing site (e.g., a double strand break) at the TRAC locus. In some embodiments, the nucleic acid encoding an anti-CD70 CAR is provided in an LNP. In some embodiments, the nucleic acid encoding an anti-CD70 CAR is provided in an expression vector, optionally an AAV vector. In some embodiments, the step (b) is performed 1, 2, or 3 days after the step (a).

In some embodiments, the method disclosed herein comprises: (a) contacting a cell with a first group of distinct LNPs comprising a first LNP comprising a nucleic acid encoding a uracil glycosylase inhibitor (UGI); a second LNP comprising the base editor and comprising a gRNA that targets the TGFBR2 locus; a third LNP comprising the base editor and comprising a gRNA that targets the HLA-A locus; a fourth LNP comprising the base editor and comprising a gRNA that targets the HLA-B locus; and a fifth LNP comprising the base editor and comprising a gRNA that targets the CIITA locus; (b) contacting a cell with (i) a second group of distinct LNPs comprising a sixth LNP comprising the base editor and comprising a gRNA that targets the CD70 locus, a seventh LNP comprising a nucleic acid encoding a UGI; and an eighth LNP comprising the RNA-guided DNA cleavase and a gRNA that targets the TRAC locus and (ii) a nucleic acid encoding an anti-CD70 CAR for insertion into an editing site (e.g., a double strand break) at the TRAC locus. In some embodiments, the nucleic acid encoding an anti-CD70 CAR is provided in an LNP. In some embodiments, the nucleic acid encoding an anti-CD70 CAR is provided in an expression vector, optionally an AAV vector. In some embodiments, the step (b) is performed 1, 2, or 3 days after the step (a).

In some embodiments, one or more compositions for multiplex gene editing in a cell comprising an anti-CD70 CAR are provided. In some embodiments, the one or more compositions comprise an HLA-A, HLA-B, TRAC, CIITA, TGFBR2, or CD70 guide RNA disclosed herein.

In some embodiments, in any of the methods and compositions disclosed herein, the HLA-A guide RNA is an HLA-A guide RNA that comprises a guide sequence disclosed herein, such as a guide sequence selected from SEQ ID NOs: 403, 404, and 412. In some embodiments, in any of the methods and compositions disclosed herein, the HLA-B guide RNA is an HLA-B guide RNA that comprises a guide sequence disclosed herein, such as a guide sequence selected from SEQ ID NOs: 405-407. In some embodiments, in any of the methods and compositions disclosed herein, the TRAC guide RNA is a TRAC guide RNA that comprises a guide sequence disclosed herein, such as a guide sequence of SEQ ID NO: 413. In some embodiments, in any of the methods and compositions disclosed herein, the TRAC guide RNA is a CIITA guide RNA that comprises a guide sequence disclosed herein, such as a guide sequence selected from SEQ ID NOs: 401, 402, and 411. In some embodiments, in any of the methods and compositions disclosed herein, the TGFBR2 guide RNA is a TGFBR2 guide RNA that comprises a guide sequence disclosed herein, such as a guide sequence selected from SEQ ID NOs: 301, 371, and 372. In some embodiments, in any of the methods and compositions disclosed herein, the TGFBR2 guide RNA is a TGFBR2 guide RNA that comprises a guide sequence disclosed herein, such as a guide sequence selected from SEQ ID NOs: 302-309. In some embodiments, in any of the methods and compositions disclosed herein, the CD70 guide RNA is a CD70 guide RNA that comprises a guide sequence disclosed herein, such as a guide sequence selected from SEQ ID NOs: 310-319.

In some embodiments, edited anti-CD70 CAR-T cells obtained by the multiplex gene editing methods or compositions are provided. In some embodiments, the edited cells comprise a genetic modification in one or more of the HLA-A, HLA-B, TRAC, CIITA, TGFBR2, or CD70 genes. In some embodiments, the genetic modification in the HLA-A gene comprises at least one nucleotide within the genomic coordinates chosen from chr6: 29942891-29942915, chr6: 29942609-29942633, and chr6: 29942864-29942884. In some embodiments, the genetic modification in the HLA-B gene comprises at least one nucleotide within the genomic coordinates chosen from chr6: 31355222-31355246, chr6: 31355221-31355245, and chr6: 31355205-31355229. In some embodiments, the genetic modification in the TRAC gene comprises at least one nucleotide within the genomic coordinates chr14: 22547524-22547544. In some embodiments, the genetic modification in the CIITA gene comprises at least one nucleotide within the genomic coordinates chosen from chr16: 10907504-10907528, chr16: 10906643-10906667, and chr16: 10906853-10906873. In some embodiments, the genetic modification in the TGFBR2 gene comprises at least one nucleotide within the genomic coordinates chosen from chr3: 30674205-30674229, chr3: 30671674-30671698, chr3: 30674167-30674191, chr3: 30671941-30671961, chr3: 30671739-30671759, chr3: 30644885-30644905, chr3: 30671618-30671638, chr3: 30671983-30672003, chr3: 30672094-30672114, chr3: 30672177-30672197, and chr3: 30674198-30674218. In some embodiments, the genetic modification in the CD70 gene comprises at least one nucleotide within the genomic coordinates chosen from chr19: 6590121-6590145, chr19: 6586268-6586292, chr19: 6590998-6591018, chr19: 6590991-6591011, chr19: 6590875-6590895, chr19: 6586396-6586416, chr19: 6586388-6586408, chr19: 6586379-6586399, chr19: 6586369-6586389, and chr19: 6586120-6586140.

### Exemplary Guide RNAs

In some embodiments, the methods and compositions provided herein further disclose HLA-A, HLA-B, TRAC, CIITA, TGFBR2, and CD70 guide RNAs useful for reducing the expression of HLA-A, HLA-B, TRAC, MHC class II, TGFBR2, and CD70 proteins on the surface of a cell comprising an anti-CD70 CAR.

In some embodiments, the guide RNA is an HLA-A guide RNA that directs an RNA-guided DNA binding agent to an HLA-A genomic target sequence and may be referred to herein as an “HLA-A guide RNA.” In some embodiments, the HLA-A guide RNA directs an RNA-guided DNA binding agent to a human HLA-A genomic target sequence. In some embodiments, the HLA-A guide RNA comprises a guide sequence selected from SEQ ID NOs: 403, 404, and 412. Further detailed description of the guide RNAs for reducing or eliminating the surface expression of HLA-A protein and for genetic modifications of HLA-A are provided in PCT/US2021/064930, PCT/US2023/068498, and PCT/US2023/068499, the entire contents of each of which is incorporated herein by reference.

In some embodiments, the methods and compositions disclosed herein comprise an HLA-A guide RNA comprising a guide sequence that targets an HLA-A genomic target sequence comprising at least 10 nucleotides within the genomic coordinates chosen from: (a) chr6: 29942864-29942884; and (b) chr6: 29942891-29942915 and chr6: 29942609-29942633. In some embodiments, the method and composition disclosed herein comprise an HLA-A guide RNA comprising a guide sequence that targets an HLA-A genomic target sequence comprising at least one nucleotide within the genomic coordinates chosen from: (a) chr6: 29942864-29942884; and (b) chr6: 29942891-29942915 and chr6: 29942609-29942633.

In some embodiments, the methods and compositions disclosed herein comprise an HLA-A guide RNA comprising a guide sequence that directs an RNA-guided DNA binding agent to induce a double stranded break (DSB) or a single-stranded break (SSB) in an HLA-A gene, wherein the HLA-A guide RNA targets and HLA-A genomic target sequence comprising at least 10 contiguous nucleotides within the genomic coordinates chosen from: (a) chr6: 29942864-29942884; and (b) chr6: 29942891-29942915 and chr6: 29942609-29942633. In some embodiments, the methods and compositions disclosed herein comprise an HLA-A guide RNA comprising a guide sequence that directs an RNA-guided DNA binding agent to induce a double stranded break (DSB) or a single-stranded break (SSB) in an HLA-A gene, wherein the HLA-A guide RNA targets an HLA-A genomic target sequence comprising at least one nucleotide within the genomic coordinates chosen from: (a) chr6: 29942864-29942884; and (b) chr6: 29942891-29942915 and chr6: 29942609-29942633.

In some embodiments, the methods and compositions disclose an HLA-A guide RNA that directs an RNA-guided DNA binding agent to induce a double stranded break (DSB) or a single-stranded break (SSB) in an HLA-A genomic target sequence. In some embodiments, the methods and compositions disclose an HLA-A guide RNA that directs an RNA-guided DNA binding agent to make a cut in an HLA-A genomic target sequence. In embodiments wherein the RNA-guided DNA cutting agent is Cas9, the cut occurs at the third base from the protospacer adjacent motif (PAM) sequence.

In some embodiments, a composition is provided comprising an HLA-A guide RNA described herein and an RNA-guided DNA binding agent or a nucleic acid encoding an RNA-guided DNA binding agent.

In some embodiments, a composition is provided comprising an HLA-A single-guide RNA (sgRNA) comprising a guide sequence that targets a genomic target comprising at least 10 contiguous nucleotides within the genomic coordinates chosen from: (a) chr6: 29942864-29942884; and (b) chr6: 29942891-29942915 and chr6: 29942609-29942633. In some embodiments, a composition is provided comprising an HLA-A sgRNA described herein and an RNA-guided DNA binding agent or a nucleic acid encoding an RNA-guided DNA binding agent.

In some embodiments, a composition is provided comprising an HLA-A dual-guide RNA (dgRNA) comprising a guide sequence that targets a genomic target comprising at least 10 contiguous nucleotides within the genomic coordinates chosen from: (a) chr6: 29942864-29942884; and (b) chr6: 29942891-29942915 and chr6: 29942609-29942633. In some embodiments, a composition is provided comprising an HLA-A dgRNA described herein and an RNA-guided DNA binding agent or a nucleic acid encoding an RNA-guided DNA binding agent.

In some embodiments, the HLA-A gRNA comprises a guide sequence of SEQ ID NOs: 412. Exemplary HLA-A guide sequences are shown below in Table 3B. In some embodiments, the HLA-A gRNA comprises a guide sequence selected from any one of SEQ ID NOs: 403 and 404. Exemplary HLA-A guide sequences are shown below in Tables 2B.

In some embodiments, the guide RNA is an HLA-B guide RNA that directs an RNA-guided DNA binding agent to an HLA-B genomic target sequence and may be referred to herein as an “HLA-B guide RNA.” In some embodiments, the HLA-B guide RNA directs an RNA-guided DNA binding agent to a human HLA-B genomic target sequence. In some embodiments, the HLA-B guide RNA comprises a guide sequence selected from SEQ ID NO: 405-407. Further detailed description of the guide RNAs for reducing or eliminating the surface expression of HLA-B protein and for genetic modifications of HLA-B are provided in PCT/US2021/064930, the entire contents of which is incorporated herein by reference.

In some embodiments, the methods and compositions disclosed herein comprise an HLA-B guide RNA comprising a guide sequence that targets an HLA-B genomic target sequence comprising at least 10 nucleotides within the genomic coordinates chosen from: chr6: 31355222-31355246, chr6: 31355221-31355245, and chr6: 31355205-31355229. In some embodiments, the method and composition disclosed herein comprise an HLA-B guide RNA comprising a guide sequence that targets an HLA-B genomic target sequence comprising at least one nucleotide within the genomic coordinates chosen from: chr6: 31355222-31355246, chr6: 31355221-31355245, and chr6: 31355205-31355229.

In some embodiments, the methods and compositions disclosed herein comprise an HLA-B guide RNA comprising a guide sequence that directs an RNA-guided DNA binding agent to induce a double stranded break (DSB) or a single-stranded break (SSB) in an HLA-B gene, wherein the HLA-B guide RNA targets and HLA-B genomic target sequence comprising at least 10 contiguous nucleotides within the genomic coordinates chosen from: chr6: 31355222-31355246, chr6: 31355221-31355245, and chr6: 31355205-31355229. In some embodiments, the methods and compositions disclosed herein comprise an HLA-B guide RNA comprising a guide sequence that directs an RNA-guided DNA binding agent to induce a double stranded break (DSB) or a single-stranded break (SSB) in an HLA-B gene, wherein the HLA-B guide RNA targets an HLA-B genomic target sequence comprising at least one nucleotide within the genomic coordinates chosen from: chr6: 31355222-31355246, chr6: 31355221-31355245, and chr6: 31355205-31355229.

In some embodiments, the methods and compositions disclose an HLA-B guide RNA that directs an RNA-guided DNA binding agent to induce a double stranded break (DSB) or a single-stranded break (SSB) in an HLA-B genomic target sequence. In some embodiments, the methods and compositions disclose an HLA-B guide RNA that directs an RNA-guided DNA binding agent to make a cut in an HLA-B genomic target sequence. In embodiments wherein the RNA-guided DNA cutting agent is Cas9, the cut occurs at the third base from the protospacer adjacent motif (PAM) sequence.

In some embodiments, a composition is provided comprising an HLA-B guide RNA described herein and an RNA-guided DNA binding agent or a nucleic acid encoding an RNA-guided DNA binding agent.

In some embodiments, a composition is provided comprising an HLA-B single-guide RNA (sgRNA) comprising a guide sequence that targets a genomic target comprising at least 10 contiguous nucleotides within the genomic coordinates chosen from: chr6: 31355222-31355246, chr6: 31355221-31355245, and chr6: 31355205-31355229. In some embodiments, a composition is provided comprising an HLA-B sgRNA described herein and an RNA-guided DNA binding agent or a nucleic acid encoding an RNA-guided DNA binding agent.

In some embodiments, a composition is provided comprising an HLA-B dual-guide RNA (dgRNA) comprising a guide sequence that targets a genomic target comprising at least 10 contiguous nucleotides within the genomic coordinates chosen from: chr6: 31355222-31355246, chr6: 31355221-31355245, and chr6: 31355205-31355229. In some embodiments, a composition is provided comprising an HLA-B dgRNA described herein and an RNA-guided DNA binding agent or a nucleic acid encoding an RNA-guided DNA binding agent.

In some embodiments, the HLA-B gRNA comprises a guide sequence selected from any one of SEQ ID NOs: 405-407. Exemplary HLA-B guide sequences are shown below in Table 2B.

In some embodiments, the guide RNA is a TRAC guide RNA that directs an RNA-guided DNA binding agent to a TRAC genomic target sequence and may be referred to herein as a “TRAC guide RNA.” In some embodiments, the TRAC guide RNA directs an RNA-guided DNA binding agent to a human TRAC genomic target sequence. In some embodiments, the TRAC guide RNA comprises a guide sequence selected from SEQ ID NO: 413. Further detailed description of the guide RNAs for reducing or eliminating the surface expression of TRAC protein and for genetic modifications of TRAC are provided in PCT/US2019/056399 and PCT/US2021/064930, the entire contents of each of which is incorporated herein by reference.

In some embodiments, the methods and compositions disclosed herein comprise a TRAC guide RNA comprising a guide sequence that targets a TRAC genomic target sequence comprising at least 10 nucleotides within the genomic coordinates: chr14: 22547524-22547544. In some embodiments, the method and composition disclosed herein comprise a TRAC guide RNA comprising a guide sequence that targets a TRAC genomic target sequence comprising at least one nucleotide within the genomic coordinates: chr14: 22547524-22547544.

In some embodiments, the methods and compositions disclosed herein comprise a TRAC guide RNA comprising a guide sequence that directs an RNA-guided DNA binding agent to induce a double stranded break (DSB) or a single-stranded break (SSB) in a TRAC gene, wherein the TRAC guide RNA targets and TRAC genomic target sequence comprising at least 10 contiguous nucleotides within the genomic coordinates: chr14: 22547524-22547544. In some embodiments, the methods and compositions disclosed herein comprise a TRAC guide RNA comprising a guide sequence that directs an RNA-guided DNA binding agent to induce a double stranded break (DSB) or a single-stranded break (SSB) in a TRAC gene, wherein the TRAC guide RNA targets a TRAC genomic target sequence comprising at least one nucleotide within the genomic coordinates: chr14: 22547524-22547544.

In some embodiments, the methods and compositions disclose a TRAC guide RNA that directs an RNA-guided DNA binding agent to induce a double stranded break (DSB) or a single-stranded break (SSB) in a TRAC genomic target sequence. In some embodiments, the methods and compositions disclose a TRAC guide RNA that directs an RNA-guided DNA binding agent to make a cut in a TRAC genomic target sequence. In embodiments wherein the RNA-guided DNA cutting agent is Cas9, the cut occurs at the third base from the protospacer adjacent motif (PAM) sequence.

In some embodiments, a composition is provided comprising a TRAC guide RNA described herein and an RNA-guided DNA binding agent or a nucleic acid encoding an RNA-guided DNA binding agent.

In some embodiments, a composition is provided comprising a TRAC single-guide RNA (sgRNA) comprising a guide sequence that targets a genomic target comprising at least 10 contiguous nucleotides within the genomic coordinates: chr14: 22547524-22547544. In some embodiments, a composition is provided comprising a TRAC sgRNA described herein and an RNA-guided DNA binding agent or a nucleic acid encoding an RNA-guided DNA binding agent.

In some embodiments, a composition is provided comprising a TRAC dual-guide RNA (dgRNA) comprising a guide sequence that targets a genomic target comprising at least 10 contiguous nucleotides within the genomic coordinates: chr14: 22547524-22547544. In some embodiments, a composition is provided comprising a TRAC dgRNA described herein and an RNA-guided DNA binding agent or a nucleic acid encoding an RNA-guided DNA binding agent.

In some embodiments, the TRAC gRNA comprises a guide sequence of SEQ ID NOs: 413. Exemplary TRAC guide sequences are shown below in Table 3B.

In some embodiments, the guide RNA is a CIITA guide RNA that directs an RNA-guided DNA binding agent to a CIITA genomic target sequence and may be referred to herein as a “CIITA guide RNA.” In some embodiments, the CIITA guide RNA directs an RNA-guided DNA binding agent to a human CIITA genomic target sequence. In some embodiments, the CIITA guide RNA comprises a guide sequence selected from SEQ ID NO: 401, 402, and 411. Further detailed description of the guide RNAs for reducing or eliminating the surface expression of MHC class II protein and for genetic modifications of CIITA are provided in PCT/US2021/064930 and PCT/US2023/068499, the entire contents of each of which is incorporated herein by reference.

In some embodiments, the methods and compositions disclosed herein comprise a CIITA guide RNA comprising a guide sequence that targets a CIITA genomic target sequence comprising at least 10 nucleotides within the genomic coordinates chosen from: (a) chr16: 10906853-10906873; and (b) chr16: 10907504-10907528 and chr16: 10906643-10906667. In some embodiments, the method and composition disclosed herein comprise a CIITA guide RNA comprising a guide sequence that targets a CIITA genomic target sequence comprising at least one nucleotide within the genomic coordinates chosen from: (a) chr16: 10906853-10906873; and (b) chr16: 10907504-10907528 and chr16: 10906643-10906667.

In some embodiments, the methods and compositions disclosed herein comprise a CIITA guide RNA comprising a guide sequence that directs an RNA-guided DNA binding agent to induce a double stranded break (DSB) or a single-stranded break (SSB) in a CIITA gene, wherein the CIITA guide RNA targets and CIITA genomic target sequence comprising at least 10 contiguous nucleotides within the genomic coordinates chosen from: (a) chr16: 10906853-10906873; and (b) chr16: 10907504-10907528 and chr16: 10906643-10906667. In some embodiments, the methods and compositions disclosed herein comprise a CIITA guide RNA comprising a guide sequence that directs an RNA-guided DNA binding agent to induce a double stranded break (DSB) or a single-stranded break (SSB) in a CIITA gene, wherein the CIITA guide RNA targets a CIITA genomic target sequence comprising at least one nucleotide within the genomic coordinates chosen from: (a) chr16: 10906853-10906873; and (b) chr16: 10907504-10907528 and chr16: 10906643-10906667.

In some embodiments, the methods and compositions disclose a CIITA guide RNA that directs an RNA-guided DNA binding agent to induce a double stranded break (DSB) or a single-stranded break (SSB) in a CIITA genomic target sequence. In some embodiments, the methods and compositions disclose a CIITA guide RNA that directs an RNA-guided DNA binding agent to make a cut in a CIITA genomic target sequence. In embodiments wherein the RNA-guided DNA cutting agent is Cas9, the cut occurs at the third base from the protospacer adjacent motif (PAM) sequence.

In some embodiments, a composition is provided comprising a CIITA guide RNA described herein and an RNA-guided DNA binding agent or a nucleic acid encoding an RNA-guided DNA binding agent.

In some embodiments, a composition is provided comprising a CIITA single-guide RNA (sgRNA) comprising a guide sequence that targets a genomic target comprising at least 10 contiguous nucleotides within the genomic coordinates chosen from: (a) chr16: 10906853-10906873; and (b) chr16: 10907504-10907528 and chr16: 10906643-10906667. In some embodiments, a composition is provided comprising a CIITA sgRNA described herein and an RNA-guided DNA binding agent or a nucleic acid encoding an RNA-guided DNA binding agent.

In some embodiments, a composition is provided comprising a CIITA dual-guide RNA (dgRNA) comprising a guide sequence that targets a genomic target comprising at least 10 contiguous nucleotides within the genomic coordinates chosen from: (a) chr16: 10906853-10906873; and (b) chr16: 10907504-10907528 and chr16: 10906643-10906667. In some embodiments, a composition is provided comprising a CIITA dgRNA described herein and an RNA-guided DNA binding agent or a nucleic acid encoding an RNA-guided DNA binding agent.

In some embodiments, the CIITA gRNA comprises a guide sequence of SEQ ID NOs: 411. Exemplary CIITA guide sequences are shown below in Table 3B. In some embodiments, the CIITA gRNA comprises a guide sequence selected from any one of SEQ ID NOs: 401 and 402. Exemplary CIITA guide sequences are shown below in Table 2B.

In some embodiments, the guide RNA is a TGFBR2 guide RNA that directs an RNA-guided DNA binding agent to a TGFBR2 genomic target sequence and may be referred to herein as a “TGFBR2 guide RNA.” In some embodiments, the TGFBR2 guide RNA directs an RNA-guided DNA binding agent to a human TGFBR2 genomic target sequence. In some embodiments, the TGFBR2 guide RNA comprises a guide sequence selected from SEQ ID NO: 301-309, 371, and 372. Further detailed description of the guide RNAs for reducing or eliminating the surface expression of TGFBR2 protein and for genetic modifications of TGFBR2 are provided in PCT/US2021/064930 and U.S. Provisional Application Nos. 63/519,733 and 63/610,602, the entire contents of each of which is incorporated herein by reference.

In some embodiments, the methods and compositions disclosed herein comprise a TGFBR2 guide RNA comprising a guide sequence that targets a TGFBR2 genomic target sequence comprising at least 10 nucleotides within the genomic coordinates chosen from: (a) chr3: 30671941-30671961, chr3: 30671739-30671759, chr3: 30644885-30644905, chr3: 30671618-30671638, chr3: 30671983-30672003, chr3: 30672094-30672114, chr3: 30672177-30672197, and chr3: 30674198-30674218; and (b) chr3: 30674205-30674229, chr3: 30671674-30671698, and chr3: 30674167-30674191. In some embodiments, the method and composition disclosed herein comprise a TGFBR2 guide RNA comprising a guide sequence that targets a TGFBR2 genomic target sequence comprising at least one nucleotide within the genomic coordinates chosen from: (a) chr3: 30671941-30671961, chr3: 30671739-30671759, chr3: 30644885-30644905, chr3: 30671618-30671638, chr3: 30671983-30672003, chr3: 30672094-30672114, chr3: 30672177-30672197, and chr3: 30674198-30674218; and (b) chr3: 30674205-30674229, chr3: 30671674-30671698, and chr3: 30674167-30674191.

In some embodiments, the methods and compositions disclosed herein comprise a TGFBR2 guide RNA comprising a guide sequence that directs an RNA-guided DNA binding agent to induce a double stranded break (DSB) or a single-stranded break (SSB) in a TGFBR2 gene, wherein the TGFBR2 guide RNA targets and TGFBR2 genomic target sequence comprising at least 10 contiguous nucleotides within the genomic coordinates chosen from: (a) chr3: 30671941-30671961, chr3: 30671739-30671759, chr3: 30644885-30644905, chr3: 30671618-30671638, chr3: 30671983-30672003, chr3: 30672094-30672114, chr3: 30672177-30672197, and chr3: 30674198-30674218; and (b) chr3: 30674205-30674229, chr3: 30671674-30671698, and chr3: 30674167-30674191. In some embodiments, the methods and compositions disclosed herein comprise a TGFBR2 guide RNA comprising a guide sequence that directs an RNA-guided DNA binding agent to induce a double stranded break (DSB) or a single-stranded break (SSB) in a TGFBR2 gene, wherein the TGFBR2 guide RNA targets a TGFBR2 genomic target sequence comprising at least one nucleotide within the genomic coordinates chosen from: (a) chr3: 30671941-30671961, chr3: 30671739-30671759, chr3: 30644885-30644905, chr3: 30671618-30671638, chr3: 30671983-30672003, chr3: 30672094-30672114, chr3: 30672177-30672197, and chr3: 30674198-30674218; and (b) chr3: 30674205-30674229, chr3: 30671674-30671698, and chr3: 30674167-30674191.

In some embodiments, the methods and compositions disclose a TGFBR2 guide RNA that directs an RNA-guided DNA binding agent to induce a double stranded break (DSB) or a single-stranded break (SSB) in a TGFBR2 genomic target sequence. In some embodiments, the methods and compositions disclose a TGFBR2 guide RNA that directs an RNA-guided DNA binding agent to make a cut in a TGFBR2 genomic target sequence. In embodiments wherein the RNA-guided DNA cutting agent is Cas9, the cut occurs at the third base from the protospacer adjacent motif (PAM) sequence.

In some embodiments, a composition is provided comprising a TGFBR2 guide RNA described herein and an RNA-guided DNA binding agent or a nucleic acid encoding an RNA-guided DNA binding agnet.

In some embodiments, a composition is provided comprising a TGFBR2 single-guide RNA (sgRNA) comprising a guide sequence that targets a genomic target comprising at least 10 contiguous nucleotides within the genomic coordinates chosen from: (a) chr3: 30671941-30671961, chr3: 30671739-30671759, chr3: 30644885-30644905, chr3: 30671618-30671638, chr3: 30671983-30672003, chr3: 30672094-30672114, chr3: 30672177-30672197, and chr3: 30674198-30674218; and (b) chr3: 30674205-30674229, chr3: 30671674-30671698, and chr3: 30674167-30674191. In some embodiments, a composition is provided comprising a TGFBR2 sgRNA described herein and an RNA-guided DNA binding agent or a nucleic acid encoding an RNA-guided DNA binding agent.

In some embodiments, a composition is provided comprising a TGFBR2 dual-guide RNA (dgRNA) comprising a guide sequence that targets a genomic target comprising at least 10 contiguous nucleotides within the genomic coordinates chosen from: (a) chr3: 30671941-30671961, chr3: 30671739-30671759, chr3: 30644885-30644905, chr3: 30671618-30671638, chr3: 30671983-30672003, chr3: 30672094-30672114, chr3: 30672177-30672197, and chr3: 30674198-30674218; and (b) chr3: 30674205-30674229, chr3: 30671674-30671698, and chr3: 30674167-30674191. In some embodiments, a composition is provided comprising a TGFBR2 dgRNA described herein and an RNA-guided DNA binding agent or a nucleic acid encoding an RNA-guided DNA binding agent.

In some embodiments, the TGFBR2 gRNA comprises a guide sequence selected from any one of SEQ ID NOs: 302-309. Exemplary TGFBR2 guide sequences are shown below in Table 3A. In some embodiments, the TGFBR2 gRNA comprises a guide sequence of SEQ ID NO: 301. Exemplary TGFBR2 guide sequences are shown below in Table 2A.

In some embodiments, the guide RNA is a CD70 guide RNA that directs an RNA-guided DNA binding agent to a CD70 genomic target sequence and may be referred to herein as a “CD70 guide RNA.” In some embodiments, the CD70 guide RNA directs an RNA-guided DNA binding agent to a human CD70 genomic target sequence. In some embodiments, the CD70 guide RNA comprises a guide sequence selected from SEQ ID NO: 310-319. Further detailed description of the guide RNAs for reducing or eliminating the surface expression of CD70 protein and for genetic modifications of CD70 are provided in PCT/US2021/064930, the entire contents of which is incorporated herein by reference.

In some embodiments, the methods and compositions disclosed herein comprise a CD70 guide RNA comprising a guide sequence that targets a CD70 genomic target sequence comprising at least 10 nucleotides within the genomic coordinates chosen from: (a) chr19: 6590998-6591018, chr19: 6590991-6591011, chr19: 6590875-6590895, 6586389, and chr19: 6586120-6586140; and (b) chr19: 6590121-6590145 and chr19: 6586268-6586292. In some embodiments, the method and composition disclosed herein comprise a CD70 guide RNA comprising a guide sequence that targets a CD70 genomic target sequence comprising at least one nucleotide within the genomic coordinates chosen from: (a) chr19: 6590998-6591018, chr19: 6590991-6591011, chr19: 6590875-6590895, chr19: 6586396-6586416, chr19: 6586388-6586408, chr19: 6586379-6586399, chr19: 6586369-6586389, and chr19: 6586120-6586140; and (b) chr19: 6590121-6590145 and chr19: 6586268-6586292.

In some embodiments, the methods and compositions disclosed herein comprise a CD70 guide RNA comprising a guide sequence that directs an RNA-guided DNA binding agent to induce a double stranded break (DSB) or a single-stranded break (SSB) in a CD70 gene, wherein the CD70 guide RNA targets and CD70 genomic target sequence comprising at least 10 contiguous nucleotides within the genomic coordinates chosen from: (a) chr19: 6590998-6591018, chr19: 6590991-6591011, chr19: 6590875-6590895, chr19: 6586396-6586416, chr19: 6586388-6586408, chr19: 6586379-6586399, chr19: 6586369-6586389, and chr19: 6586120-6586140; and (b) chr19: 6590121-6590145 and chr19: 6586268-6586292. In some embodiments, the methods and compositions disclosed herein comprise a CD70 guide RNA comprising a guide sequence that directs an RNA-guided DNA binding agent to induce a double stranded break (DSB) or a single-stranded break (SSB) in a CD70 gene, wherein the CD70 guide RNA targets a CD70 genomic target sequence comprising at least one nucleotide within the genomic coordinates chosen from: (a) chr19: 6590998-6591018, chr19: 6590991-6591011, chr19: 6590875-6590895, chr19: 6586396-6586416, chr19: 6586388-6586408, chr19: 6586379-6586399, chr19: 6586369-6586389, and chr19: 6586120-6586140; and (b) chr19: 6590121-6590145 and chr19: 6586268-6586292.

In some embodiments, the methods and compositions disclose a CD70 guide RNA that directs an RNA-guided DNA binding agent to induce a double stranded break (DSB) or a single-stranded break (SSB) in a CD70 genomic target sequence. In some embodiments, the methods and compositions disclose a CD70 guide RNA that directs an RNA-guided DNA binding agent to make a cut in a CD70 genomic target sequence. In embodiments wherein the RNA-guided DNA cutting agent is Cas9, the cut occurs at the third base from the protospacer adjacent motif (PAM) sequence.

In some embodiments, a composition is provided comprising a CD70 guide RNA described herein and an RNA-guided DNA binding agent or a nucleic acid encoding an RNA-guided DNA binding agent.

In some embodiments, a composition is provided comprising a CD70 single-guide RNA (sgRNA) comprising a guide sequence that targets a genomic target comprising at least 10 contiguous nucleotides within the genomic coordinates chosen from: (a) (a) chr19: 6590998-6591018, chr19: 6590991-6591011, chr19: 6590875-6590895, chr19: 6586396-6586416, chr19: 6586388-6586408, chr19: 6586379-6586399, chr19: 6586369-6586389, and chr19: 6586120-6586140; and (b) chr19: 6590121-6590145 and chr19: 6586268-6586292. In some embodiments, a composition is provided comprising a CD70 sgRNA described herein and an RNA-guided DNA binding agent or a nucleic acid encoding an RNA-guided DNA binding agent.

In some embodiments, a composition is provided comprising a CD70 dual-guide RNA (dgRNA) comprising a guide sequence that targets a genomic target comprising at least 10 contiguous nucleotides within the genomic coordinates chosen from: (a) chr19: 6590998-6591018, chr19: 6590991-6591011, chr19: 6590875-6590895, chr19: 6586396-6586416, chr19: 6586388-6586408, chr19: 6586379-6586399, chr19: 6586369-6586389, and chr19: 6586120-6586140; and (b) chr19: 6590121-6590145 and chr19: 6586268-6586292. In some embodiments, a composition is provided comprising a CD70 dgRNA described herein and an RNA-guided DNA binding agent or a nucleic acid encoding an RNA-guided DNA binding agent.

In some embodiments, the CD70 gRNA comprises a guide sequence selected from any one of SEQ ID NOs: 312-319. Exemplary CD70 guide sequences are shown below in Table 3A. In some embodiments, the CD70 gRNA comprises a guide sequence selected from any one of SEQ ID NOs: 310 and 311. Exemplary CD70 guide sequences are shown below in Table 2A.

TABLE 2A
Exemplary Nme guide RNAs

| Guide | | Genomic | Guide | full guide RNA | |
|---|---|---|---|---|---|
| ID | Target | Coordinates | sequence | sequence | modified guide RNA sequence |
| G029855 | TGFBR2 | chr3: | CGAGAUG | CGAGAUGUCAUUUCCCAG | mC*mG*mA*mGmAUGmUmCAmUUmUCCCAmG |
| | | 30674205- | UCAUUUC | AGCACCGUUGUAGCUCCC | AGCmACCmGUUGmUmAmGmCUCCCmUmGmAm |
| | | 30674229 | CCAGAGC | UGAAACCGUUGCUACAAU | AmAmCmCGUUmGmCUAmCAAU*AAGmGmCCm |
| | | | ACC | AAGGCCGUCGAAAGAUGU | GmUmCmGmAmAmAmGmAmUGUGCmCGmCAAm |
| | | | (SEQ ID | GCCGCAACGCUCUGCCUU | CGCUCUmGmCCmUmUmCmUGGCAUCG*mU*m |
| | | | NO: | CUGGCAUCGUU (SEQ | U (SEQ ID NO: 341) |
| | | | 301) | ID NO: 321) | |
| G034983 | TGFBR2 | chr3: | CGAGAUG | CGAGAUGUCAUUUCCCAG | mC*mG*mA *mGmAUGmUmCAmUUmUCCCAmG |
| | | 30674205- | UCAUUUC | AGCACCGUUGUAGCUCCC | AGCmACCmGUUGmUmAmGmCUCCCmUmGmAm |
| | | 30674229 | CCAGAGC | UGAAACCGUUGCUACAAU | AmAmCmCGUUmGmCUAmCAAUAAGmGmCCmG |
| | | | ACC | AAGGCCGUCGAAAGAUGU | mUmCmGmAmAmAmGmAmUGUGCmCGmCAAmC |
| | | | (SEQ ID | GCCGCAACGCUCUGCCUU | GCUCUmGmCCmUmUmCmUGGCAUCG*mU*mU |
| | | | NO: | CUGGCAUCGUU (SEQ | (SEQ ID NO: 342) |
| | | | 301) | ID NO: 321) | |
| G033267 | CD70 | chr19: | CUCAGCU | CUCAGCUACGUCCCACUG | mC*mU*mC*mAmGCUmAmCGmUCmCCACUmG |
| | | 6590121- | ACGUCCC | GAAGAAGUUGUAGCUCCC | GAAmGAAmGUUGmUmAmGmCUCCCmUmGmAm |
| | | 6590145 | ACUGGAA | UGAAACCGUUGCUACAAU | AmAmCmCGUUmGmCUAmCAAU*AAGmGmCCm |
| | | | GAA | AAGGCCGUCGAAAGAUGU | GmUmCmGmAmAmAmGmAmUGUGCmCGmCAAm |
| | | | (SEQ ID | GCCGCAACGCUCUGCCUU | CGCUCUmGmCCmUmUmCmUGGCAUCG*mU*m |
| | | | NO: | CUGGCAUCGUU (SEQ | U (SEQ ID NO: 353) |
| | | | 310) | ID NO: 330) | |
| G034199 | CD70 | chr19: | CUCAGCU | CUCAGCUACGUCCCACUG | mC *mU*mC*mAmGCUmAmCGmUCmCCACUmG |
| | | 6590121- | ACGUCCC | GAAGAAGUUGUAGCUCCC | GAAmGAAmGUUGmUmAmGmCUCCCmUmGmAm |
| | | 6590145 | ACUGGAA | UGAAACCGUUGCUACAAU | AmAmCmCGUUmGmCUAmCAAUAAGmGmCCmG |
| | | | GAA | AAGGCCGUCGAAAGAUGU | mUmCmGmAmAmAmGmAmUGUGCmCGmCAAmC |
| | | | (SEQ ID | GCCGCAACGCUCUGCCUU | GCUCUmGmCCmUmUmCmUGGCAUCG*mU*mU |
| | | | NO: | CUGGCAUCGUU (SEQ | (SEQ ID NO: 354) |
| | | | 310) | ID NO: 330) | |
| G033276 | CD70 | chr19: | UACAUGG | UACAUGGUACACAUCCAG | mU*mA*mC*mAmUGGmUmACmACmAUCCAmG |
| | | 6586268- | UACACAU | GUGACGGUUGUAGCUCCC | GUGmACGmGUUGmUmAmGmCUCCCmUmGmAm |
| | | 6586292 | CCAGGUG | UGAAACCGUUGCUACAAU | AmAmCmCGUUmGmCUAmCAAU*AAGmGmCCm |
| | | | ACG | AAGGCCGUCGAAAGAUGU | GmUmCmGmAmAmAmGmAmUGUGCmCGmCAAm |
| | | | (SEQ ID | GCCGCAACGCUCUGCCUU | CGCUCUmGmCCmUmUmCmUGGCAUCG*mU*m |
| | | | NO: | CUGGCAUCGUU (SEQ | U (SEQ ID NO: 355) |
| | | | 311) | ID NO: 331) | |
| G034200 | CD70 | chr19: | UACAUGG | UACAUGGUACACAUCCAG | mU*mA*mC*mAmUGGmUmACmACmAUCCAmG |
| | | 6586268- | UACACAU | GUGACGGUUGUAGCUCCC | GUGmACGmGUUGmUmAmGmCUCCCmUmGmAm |
| | | 6586292 | CCAGGUG | UGAAACCGUUGCUACAAU | AmAmCmCGUUmGmCUAmCAAUAAGmGmCCmG |
| | | | ACG | AAGGCCGUCGAAAGAUGU | mUmCmGmAmAmAmGmAmUGUGCmCGmCAAmC |
| | | | (SEQ ID | GCCGCAACGCUCUGCCUU | GCUCUmGmCCmUmUmCmUGGCAUCG*mU*mU |
| | | | NO: | CUGGCAUCGUU (SEQ | (SEQ ID NO: 356) |
| | | | 311) | ID NO: 331) | |

TABLE 2B
Exemplary Nme guide RNA

| Guide | | Genomic | Guide | full guide | modified guide RNA |
|---|---|---|---|---|---|
| ID | Target | Coordinates | sequence | RNA sequence | sequence |
| G026584 | CIITA | chr16: | UCAAAGU | UCAAAGUACCCU | mU*mC*mA *mAmAGUmAmC |
| | | 10907504- | ACCCUAC | ACAGGAGGACCA | CmCUmACAGGmAGGAmCCA |
| | | 10907528 | AGGAGGA | GUUGUAGCUCCC | mGUUGmUmAmGmCUCCCmU |
| | | | CCA | UGAAACCGUUGC | mGmAmAmAmCmCGUUmGmC |
| | | | (SEQ ID | UACAAUAAGGCC | UAmCAAU*AAGmGmCCmGm |
| | | | NO: | GUCGAAAGAUGU | UmCmGmAmAmAmGmAmUGU |
| | | | 401) | GCCGCAACGCUC | GCmCGmCAAmCGCUCUmGm |
| | | | | UGCCUUCUGGCA | CCmUmUmCmUGGCAUCG*m |
| | | | | UCGUU (SEQ | U*mU (SEQ ID NO: |
| | | | | ID NO: 421) | 441) |
| G034201 | CIITA | chr16: | UCAAAGU | UCAAAGUACCCU | mU*mC*mA *mAmAGUmAmC |
| | | 10907504- | ACCCUAC | ACAGGAGGACCA | CmCUmACAGGmAGGAmCCA |
| | | 10907528 | AGGAGGA | GUUGUAGCUCCC | mGUUGmUmAmGmCUCCCmU |
| | | | CCA | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 401) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 421) | 442) |
| G029131 | CIITA | chr16: | AGCUGCC | AGCUGCCGUUCU | mA *mG*mC *mUmGCCmGmU |
| | | 10906643- | GUUCUGC | GCCCAGUCCGGG | UmCUmGCCCAmGUCCmGGG |
| | | 10906667 | CCAGUCC | GUUGUAGCUCCC | mGUUGmUmAmGmCUCCCmU |
| | | | GGG | UGAAACCGUUGC | mGmAmAmAmCmCGUUmGmC |
| | | | (SEQ ID | UACAAUAAGGCC | UAmCAAU*AAGmGmCCmGm |
| | | | NO: | GUCGAAAGAUGU | UmCmGmAmAmAmGmAmUGU |
| | | | 402) | GCCGCAACGCUC | GCmCGmCAAmCGCUCUmGm |
| | | | | UGCCUUCUGGCA | CCmUmUmCmUGGCAUCG*m |
| | | | | UCGUU (SEQ | U*mU (SEQ ID NO: |
| | | | | ID NO: 422) | 443) |
| G034619 | CIITA | chr16: | AGCUGCC | AGCUGCCGUUCU | mA*mG*mC *mUmGCCmGmU |
| | | 10906643- | GUUCUGC | GCCCAGUCCGGG | UmCUmGCCCAmGUCCmGGG |
| | | 10906667 | CCAGUCC | GUUGUAGCUCCC | mGUUGmUmAmGmCUCCCmU |
| | | | GGG | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 402) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 422) | 444) |
| G028918 | HLA-A | chr6: | GCUCUAU | GCUCUAUCCACG | mG*mC*mU*mCmUAUmCmC |
| | | 29942891- | CCACGGC | GCGCCCGCGGCU | AmCGmGCGCCmCGCGmGCU |
| | | 29942915 | GCCCGCG | GUUGUAGCUCCC | mGUUGmUmAmGmCUCCCmU |
| | | | GCU | UGAAACCGUUGC | mGmAmAmAmCmCGUUmGmC |
| | | | (SEQ ID | UACAAUAAGGCC | UAmCAAU*AAGmGmCCmGm |
| | | | NO: | GUCGAAAGAUGU | UmCmGmAmAmAmGmAmUGU |
| | | | 403) | GCCGCAACGCUC | GCmCGmCAAmCGCUCUmGm |
| | | | | UGCCUUCUGGCA | CCmUmUmCmUGGCAUCG*m |
| | | | | UCGUU (SEQ | U*mU (SEQ ID NO: |
| | | | | ID NO: 423) | 445) |
| G034202 | HLA-A | chr6: | GCUCUAU | GCUCUAUCCACG | mG*mC*mU*mCmUAUmCmC |
| | | 29942891- | CCACGGC | GCGCCCGCGGCU | AmCGmGCGCCmCGCGmGCU |
| | | 29942915 | GCCCGCG | GUUGUAGCUCCC | mGUUGmUmAmGmCUCCCmU |
| | | | GCU | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 403) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 423) | 446) |
| G028913 | HLA-A | chr6: | CACUCAC | CACUCACCCGCC | mC *mA*mC *mUmCACmCmC |
| | | 29942609- | CCGCCCA | CAGGUCUGGGUC | GmCCmCAGGUmCUGGmGUC |
| | | 29942633 | GGUCUGG | GUUGUAGCUCCC | mGUUGmUmAmGmCUCCCmU |
| | | | GUC | UGAAACCGUUGC | mGmAmAmAmCmCGUUmGmC |
| | | | (SEQ ID | UACAAUAAGGCC | UAmCAAU*AAGmGmCCmGm |
| | | | NO: | GUCGAAAGAUGU | UmCmGmAmAmAmGmAmUGU |
| | | | 404) | GCCGCAACGCUC | GCmCGmCAAmCGCUCUmGm |
| | | | | UGCCUUCUGGCA | CCmUmUmCmUGGCAUCG*m |
| | | | | UCGUU (SEQ | U*mU (SEQ ID NO: |
| | | | | ID NO: 424) | 447) |
| G034617 | HLA-A | chr6: | CACUCAC | CACUCACCCGCC | mC *mA*mC *mUmCACmCmC |
| | | 29942609- | CCGCCCA | CAGGUCUGGGUC | GmCCmCAGGUmCUGGmGUC |
| | | 29942633 | GGUCUGG | GUUGUAGCUCCC | mGUUGmUmAmGmCUCCCmU |
| | | | GUC | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 404) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 424) | 448) |
| G032794 | HLA-B | chr6: | UCUGGGA | UCUGGGAAAGGA | mU*mC*mU*mGmGGAmAmA |
| | | 31355222- | AAGGAGG | GGGGAAGAUGAG | GmGAmGGGGAmAGAUmGAG |
| | | 31355246 | GGAAGAU | GUUGUAGCUCCC | mGUUGmUmAmGmCUCCCmU |
| | | | GAG | UGAAACCGUUGC | mGmAmAmAmCmCGUUmGmC |
| | | | (SEQ ID | UACAAUAAGGCC | UAmCAAU*AAGmGmCCmGm |
| | | | NO: | GUCGAAAGAUGU | UmCmGmAmAmAmGmAmUGU |
| | | | 406) | GCCGCAACGCUC | GCmCGmCAAmCGCUCUmGm |
| | | | | UGCCUUCUGGCA | CCmUmUmCmUGGCAUCG*m |
| | | | | UCGUU (SEQ | U*mU (SEQ ID NO: |
| | | | | ID NO: 426) | 451) |
| G034208 | HLA-B | chr6: | UCUGGGA | UCUGGGAAAGGA | mU*mC*mU*mGmGGAmAmA |
| | | 31355222- | AAGGAGG | GGGGAAGAUGAG | GmGAmGGGGAmAGAUmGAG |
| | | 31355246 | GGAAGAU | GUUGUAGCUCCC | mGUUGmUmAmGmCUCCCmU |
| | | | GAG | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 406) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 426) | 452) |
| G032795 | HLA-B | chr6: | CUCUGGG | CUCUGGGAAAGG | mC *mU*mC *mUmGGGmAmA |
| | | 31355221- | AAAGGAG | AGGGGAAGAUGA | AmGGmAGGGGmAAGAmUGA |
| | | 31355245 | GGGAAGA | GUUGUAGCUCCC | mGUUGmUmAmGmCUCCCmU |
| | | | UGA | UGAAACCGUUGC | mGmAmAmAmCmCGUUmGmC |
| | | | (SEQ ID | UACAAUAAGGCC | UAmCAAU*AAGmGmCCmGm |
| | | | NO: | GUCGAAAGAUGU | UmCmGmAmAmAmGmAmUGU |
| | | | 405) | GCCGCAACGCUC | GCmCGmCAAmCGCUCUmGm |
| | | | | UGCCUUCUGGCA | CCmUmUmCmUGGCAUCG*m |
| | | | | UCGUU (SEQ | U*mU (SEQ ID NO: |
| | | | | ID NO: 425) | 449) |
| G034209 | HLA-B | chr6: | CUCUGGG | CUCUGGGAAAGG | mC *mU*mC *mUmGGGmAmA |
| | | 31355221- | AAAGGAG | AGGGGAAGAUGA | AmGGmAGGGGmAAGAmUGA |
| | | 31355245 | GGGAAGA | GUUGUAGCUCCC | mGUUGmUmAmGmCUCCCmU |
| | | | UGA | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 405) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 425) | 450) |
| G032806 | HLA-B | chr6: | UCCCAGA | UCCCAGAGCCGU | mU*mC*mC*mCmAGAmGmC |
| | | 31355205- | GCCGUCU | CUUCCCAGUCCA | CmGUmCUUCCmCAGUmCCA |
| | | 31355229 | UCCCAGU | GUUGUAGCUCCC | mGUUGmUmAmGmCUCCCmU |
| | | | CCA | UGAAACCGUUGC | mGmAmAmAmCmCGUUmGmC |
| | | | (SEQ ID | UACAAUAAGGCC | UAmCAAU*AAGmGmCCmGm |
| | | | NO: | GUCGAAAGAUGU | UmCmGmAmAmAmGmAmUGU |
| | | | 407) | GCCGCAACGCUC | GCmCGmCAAmCGCUCUmGm |
| | | | | UGCCUUCUGGCA | CCmUmUmCmUGGCAUCG*m |
| | | | | UCGUU (SEQ | U*mU (SEQ ID NO: |
| | | | | ID NO: 427) | 453) |
| G034211 | HLA-B | chr6: | UCCCAGA | UCCCAGAGCCGU | mU*mC*mC*mCmAGAmGmC |
| | | 31355205- | GCCGUCU | CUUCCCAGUCCA | CmGUmCUUCCmCAGUmCCA |
| | | 31355229 | UCCCAGU | GUUGUAGCUCCC | mGUUGmUmAmGmCUCCCmU |
| | | | CCA | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 407) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 427) | 454) |

TABLE 3A
Exemplary Spy guide RNAs

| Guide | | Genomic | Guide | full guide | modified guide RNA |
|---|---|---|---|---|---|
| ID | Target | Coordinates | sequence | RNA sequence | sequence |
| G021083 | TGFBR2 | chr3: | UCGCUUU | UCGCUUUGCUGA | mU*mC*mG*CUUUGCUGAGG |
| | | 30671941- | GCUGAGG | GGUCUAUAGUUU | UCUAUAGUUUUAGAmGmCmU |
| | | 30671961 | UCUAUA | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCAmAmCmUmUmGmAmA |
| | | | 302) | CGUUAUCAACUU | mAmAmAmGmUmGmGmCmAmC |
| | | | | GAAAAAGUGGCA | mCmGmAmGmUmCmGmGmUmG |
| | | | | CCGAGUCGGUGC | mCmU*mU*mU*mU (SEQ |
| | | | | UUUU (SEQ ID | ID NO: 343) |
| | | | | NO: 322) | |
| G029528 | TGFBR2 | chr3: | UCGCUUU | UGGGAGGACCUG | mU*mG*mG*GAGGACCUGCG |
| | | 30671941- | GCUGAGG | CGCAAGCUGUUU | CAAGCUGUUUUAGAmGmCmU |
| | | 30671961 | UCUAUA | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCACGAAAGGGCACCGA |
| | | | 302) | CGUUAUCACGAA | GUCGGmU*mG*mC*mU |
| | | | | AGGGCACCGAGU | (SEQ ID NO: 344) |
| | | | | CGGUGCU (SEQ | |
| | | | | ID NO: 992) | |
| G021108 | TGFBR2 | chr3: | UCUACUG | UCUACUGCUACC | mU*mC*mU*ACUGCUACCGC |
| | | 30671739- | CUACCGC | GCGUUAACGUUU | GUUAACGUUUUAGAmGmCmU |
| | | 30671759 | GUUAAC | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCAmAmCmUmUmGmAmA |
| | | | 303) | CGUUAUCAACUU | mAmAmAmGmUmGmGmCmAmC |
| | | | | GAAAAAGUGGCA | mCmGmAmGmUmCmGmGmUmG |
| | | | | CCGAGUCGGUGC | mCmU*mU*mU*mU (SEQ |
| | | | | UUUU (SEQ ID | ID NO: 345) |
| | | | | NO: 323) | |
| G029526 | TGFBR2 | chr3: | UCUACUG | UCUACUGCUACC | mU*mC*mU*ACUGCUACCGC |
| | | 30671739- | CUACCGC | GCGUUAACGUUU | GUUAACGUUUUAGAmGmCmU |
| | | 30671759 | GUUAAC | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCACGAAAGGGCACCGA |
| | | | 303) | CGUUAUCACGAA | GUCGGmU*mG*mC*mU |
| | | | | AGGGCACCGAGU | (SEQ ID NO: 346) |
| | | | | CGGUGCU (SEQ | |
| | | | | ID NO: 993) | |
| G028024 | TGFBR2 | chr3: | GAAGCCA | GAAGCCACAGGA | mG*mA*mA*GCCACAGGAAG |
| | | 30644885- | CAGGAAG | AGUCUGUGGUUU | UCUGUGGUUUUAGAmGmCmU |
| | | 30644905 | UCUGUG | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCACGAAAGGGCACCGA |
| | | | 304) | CGUUAUCACGAA | GUCGGmU*mG*mC*mU |
| | | | | AGGGCACCGAGU | (SEQ ID NO: 347) |
| | | | | CGGUGCU (SEQ | |
| | | | | ID NO: 324) | |
| G028027 | TGFBR2 | chr3: | UCUGAUG | UCUGAUGGGGAA | mU*mC*mU*GAUGGGGAAAC |
| | | 30671618- | GGGAAAC | ACAAAACAGUUU | AAAACAGUUUUAGAmGmCmU |
| | | 30671638 | AAAACA | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCACGAAAGGGCACCGA |
| | | | 305) | CGUUAUCACGAA | GUCGGmU*mG*mC*mU |
| | | | | AGGGCACCGAGU | (SEQ ID NO: 348) |
| | | | | CGGUGCU (SEQ | |
| | | | | ID NO: 325) | |
| G028030 | TGFBR2 | chr3: | UUCAGAG | UUCAGAGCAGUU | mU*mU*mC*AGAGCAGUUUG |
| | | 30671983- | CAGUUUG | UGAGACAGGUUU | AGACAGGUUUUAGAmGmCmU |
| | | 30672003 | AGACAG | UAGAGCUAGAAA | mAmGmAmAmAmUmAmGmCAA |
| | | | (SID | UAGCAAGUUAAA | GUUAAAAUAAGGCUAGUCCG |
| | | | NO: | AUAAGGCUAGUC | UUAUCACGAAAGGGCACCGA |
| | | | 306) | CGUUAUCACGAA | GUCGGmU*mG*mC*mU |
| | | | | AGGGCACCGAGU | (SEQ ID NO: 349) |
| | | | | CGGUGCU (SEQ | |
| | | | | ID NO: 326) | |
| G028034 | TGFBR2 | chr3: | ACUCCAG | ACUCCAGUUCCU | mA*mC*mU*CCAGUUCCUGA |
| | | 30672094- | UUCCUGA | GACGGCUGGUUU | CGGCUGGUUUUAGAmGmCmU |
| | | 30672114 | CGGCUG | UAGAGCUAGAAA | mAmGmAmAmAmUmAmGmCAA |
| | | | (SID | UAGCAAGUUAAA | GUUAAAAUAAGGCUAGUCCG |
| | | | NO: | AUAAGGCUAGUC | UUAUCACGAAAGGGCACCGA |
| | | | 307) | CGUUAUCACGAA | GUCGGmU*mG*mC*mU |
| | | | | AGGGCACCGAGU | (SEQ ID NO: 350) |
| | | | | CGGUGCU (SEQ | |
| | | | | ID NO: 327) | |
| G028036 | TGFBR2 | chr3: | ACCUACA | ACCUACAGGAGU | mA*mC*mC*UACAGGAGUAC |
| | | 30672177- | GGAGUAC | ACCUGACGGUUU | CUGACGGUUUUAGAmGmCmU |
| | | 30672197 | CUGACG | UAGAGCUAGAAA | mAmGmAmAmAmUmAmGmCAA |
| | | | (SID | UAGCAAGUUAAA | GUUAAAAUAAGGCUAGUCCG |
| | | | NO: | AUAAGGCUAGUC | UUAUCACGAAAGGGCACCGA |
| | | | 308) | CGUUAUCACGAA | GUCGGmU*mG*mC*mU |
| | | | | AGGGCACCGAGU | (SEQ ID NO: 351) |
| | | | | CGGUGCU (SEQ | |
| | | | | ID NO: 328) | |
| G028041 | TGFBR2 | chr3: | UUCCCAG | UUCCCAGAGCAC | mU*mU*mC*CCAGAGCACCA |
| | | 30674198- | AGCACCA | CAGAGCCAGUUU | GAGCCAGUUUUAGAmGmCmU |
| | | 30674218 | GAGCCA | UAGAGCUAGAAA | mAmGmAmAmAmUmAmGmCAA |
| | | | (SID | UAGCAAGUUAAA | GUUAAAAUAAGGCUAGUCCG |
| | | | NO: | AUAAGGCUAGUC | UUAUCACGAAAGGGCACCGA |
| | | | 309) | CGUUAUCACGAA | GUCGGmU*mG*mC*mU |
| | | | | AGGGCACCGAGU | (SEQ ID NO: 352) |
| | | | | CGGUGCU (SEQ | |
| | | | | ID NO: 329) | |
| G026733 | CD70 | chr19: | GGUGAUC | GGUGAUCGCCGC | mG*mG*mU*GAUCGCCGCGG |
| | | 6590998- | GCCGCGG | GGCGAUGCGUUU | CGAUGCGUUUUAGAmGmCmU |
| | | 6591018 | CGAUGC | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCAmAmCmUmUmGmAmA |
| | | | 312) | CGUUAUCAACUU | mAmAmAmGmUmGmGmCmAmC |
| | | | | GAAAAAGUGGCA | mCmGmAmGmUmCmGmGmUmG |
| | | | | CCGAGUCGGUGC | mCmU*mU*mU*mU (SEQ |
| | | | | UUUU (SEQ ID | ID NO: 357) |
| | | | | NO: 332) | |
| G024836 | CD70 | chr19: | GGUGAUC | GGUGAUCGCCGC | mG*mG*mU*GAUCGCCGCGG |
| | | 6590998- | GCCGCGG | GGCGAUGCGUUU | CGAUGCGUUUUAGAmGmCmU |
| | | 6591018 | CGAUGC | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCACGAAAGGGCACCGA |
| | | | 312) | CGUUAUCACGAA | GUCGGmU*mG*mC*mU |
| | | | | AGGGCACCGAGU | (SEQ ID NO: 358) |
| | | | | CGGUGCU (SEQ | |
| | | | | ID NO: 994) | |
| G026736 | CD70 | chr19: | GCCGCGG | GCCGCGGCGAUG | mG*mC*mC*GCGGCGAUGCC |
| | | 6590991- | CGAUGCC | CCGGAGGAGUUU | GGAGGAGUUUUAGAmGmCmU |
| | | 6591011 | GGAGGA | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCAmAmCmUmUmGmAmA |
| | | | 313) | CGUUAUCAACUU | mAmAmAmGmUmGmGmCmAmC |
| | | | | GAAAAAGUGGCA | mCmGmAmGmUmCmGmGmUmG |
| | | | | CCGAGUCGGUGC | mCmU*mU*mU*mU (SEQ |
| | | | | UUUU (SEQ ID | ID NO: 359) |
| | | | | NO: 333) | |
| G024839 | CD70 | chr19: | GCCGCGG | GCCGCGGCGAUG | mG*mC*mC*GCGGCGAUGCC |
| | | 6590991- | CGAUGCC | CCGGAGGAGUUU | GGAGGAGUUUUAGAmGmCmU |
| | | 6591011 | GGAGGA | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCACGAAAGGGCACCGA |
| | | | 313) | CGUUAUCACGAA | GUCGGmU*mG*mC*mU |
| | | | | AGGGCACCGAGU | (SEQ ID NO: 360) |
| | | | | CGGUGCU (SEQ | |
| | | | | ID NO: 995) | |
| G026760 | CD70 | chr19: | GUGCAUC | GUGCAUCCAGCG | mG*mU*G*CAUCCAGCGCU |
| | | 6590875- | CAGCGCU | CUUCGCACGUUU | UCGCACGUUUUAGAmGmCmU |
| | | 6590895 | UCGCAC | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCACGAAAGGGCACCGA |
| | | | 314) | CGUUAUCACGAA | GUCGGmU*mG*mC*mU |
| | | | | AGGGCACCGAGU | (SEQ ID NO: 361) |
| | | | | CGGUGCU (SEQ | |
| | | | | ID NO: 334) | |
| G028064 | CD70 | chr19: | GCUGAGG | GCUGAGGUCCUG | mG*mC*mU*GAGGUCCUGGG |
| | | 6586396- | UCCUGGG | GGGGCACAGUUU | GGCACAGUUUUAGAmGmCmU |
| | | 6586416 | GGCACA | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCACGAAAGGGCACCGA |
| | | | 315) | CGUUAUCACGAA | GUCGGmU*mG*mC*mU |
| | | | | AGGGCACCGAGU | (SEQ ID NO: 362) |
| | | | | CGGUGCU (SEQ | |
| | | | | ID NO: 335) | |
| G028067 | CD70 | chr19: | CAGGACC | CAGGACCUCAGC | mC*mA*mG*GACCUCAGCAG |
| | | 6586388- | UCAGCAG | AGGACCCCGUUU | GACCCCGUUUUAGAmGmCmU |
| | | 6586408 | GACCCC | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCACGAAAGGGCACCGA |
| | | | 316) | CGUUAUCACGAA | GUCGGmU*mG*mC*mU |
| | | | | AGGGCACCGAGU | (SEQ ID NO: 363) |
| | | | | CGGUGCU (SEQ | |
| | | | | ID NO: 336) | |
| G028069 | CD70 | chr19: | AGCAGGA | AGCAGGACCCCA | mA*mG*mC*AGGACCCCAGG |
| | | 6586379- | CCCCAGG | GGCUAUACGUUU | CUAUACGUUUUAGAmGmCmU |
| | | 6586399 | CUAUAC | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCACGAAAGGGCACCGA |
| | | | 317) | CGUUAUCACGAA | GUCGGmU*mG*mC*mU |
| | | | | AGGGCACCGAGU | (SEQ ID NO: 364) |
| | | | | CGGUGCU (SEQ | |
| | | | | ID NO: 337) | |
| G028071 | CD70 | chr19: | CCCCCUG | CCCCCUGCCAGU | mC*mC*mC*CCUGCCAGUAU |
| | | 6586369- | CCAGUAU | AUAGCCUGGUUU | AGCCUGGUUUUAGAmGmCmU |
| | | 6586389 | AGCCUG | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCACGAAAGGGCACCGA |
| | | | 318) | CGUUAUCACGAA | GUCGGmU*mG*mC*mU |
| | | | | AGGGCACCGAGU | (SEQ ID NO: 365) |
| | | | | CGGUGCU (SEQ | |
| | | | | ID NO: 338) | |
| G028075 | CD70 | chr19: | CUCCCAG | CUCCCAGCGCCU | mC*mU*mC*CCAGCGCCUGA |
| | | 6586120- | CGCCUGA | GACGCCCCGUUU | CGCCCCGUUUUAGAmGmCmU |
| | | 6586140 | CGCCCC | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCACGAAAGGGCACCGA |
| | | | 319) | CGUUAUCACGAA | GUCGGmU*mG*mC*mU |
| | | | | AGGGCACCGAGU | (SEQ ID NO: 366) |
| | | | | CGGUGCU (SEQ | |
| | | | | ID NO: 339) | |

TABLE 3B
Exemplary Spy guide RNAs

| Guide | | Genomic | Guide | full guide | modified guide RNA |
|---|---|---|---|---|---|
| ID | Target | Coordinates | sequence | RNA sequence | sequence |
| G013675 | CIITA | Chr16: | CCCCCGG | CCCCCGGACGGU | mC*mC*mC*CCGGACGGUUC |
| | | 10906853- | ACGGUUC | UCAAGCAAGUUU | AAGCAAGUUUUAGAmGmCmU |
| | | 10906873 | AAGCAA | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCAmAmCmUmUmGmAmA |
| | | | 411) | CGUUAUCAACUU | mAmAmAmGmUmGmGmCmAmC |
| | | | | GAAAAAGUGGCA | mCmGmAmGmUmCmGmGmUmG |
| | | | | CCGAGUCGGUGC | mCmU*mU*mU*mU (SEQ |
| | | | | UUUU (SEQ ID | ID NO: 461) |
| | | | | NO: 431) | |
| G018995 | HLA-A | Chr6: | ACAGCGA | ACAGCGACGCCG | mA*mC*AmG*CGACGCCGCG |
| | | 29942864- | CGCCGCG | CGAGCCAGGUUU | AGCCAGGUUUUAGAmGmCmU |
| | | 29942884 | AGCCAG | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCAmAmCmUmUmGmAmA |
| | | | 412) | CGUUAUCAACUU | mAmAmAmGmUmGmGmCmAmC |
| | | | | GAAAAAGUGGCA | mCmGmAmGmUmCmGmGmUmG |
| | | | | CCGAGUCGGUGC | mCmU*mU*mU*mU (SEQ |
| | | | | UUUU (SEQ ID | ID NO: 462) |
| | | | | NO: 432) | |
| G013006 | TRAC | Chr14: | CUCUCAG | CUCUCAGCUGGU | mC*mU*mC*UCAGCUGGUAC |
| | | 22547524- | CUGGUAC | ACACGGCAGUUU | ACGGCAGUUUUAGAmGmCmU |
| | | 22547544 | ACGGCA | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCAmAmCmUmUmGmAmA |
| | | | 413) | CGUUAUCAACUU | mAmAmAmGmUmGmGmCmAmC |
| | | | | GAAAAAGUGGCA | mCmGmAmGmUmCmGmGmUmG |
| | | | | CCGAGUCGGUGC | mCmU*mU*mU*mU (SEQ |
| | | | | UUUU (SEQ ID | ID NO: 463) |
| | | | | NO: 433) | |
| G025420 | TRAC | Chr14: | CUCUCAG | CUCUCAGCUGGU | mC*mU*mC*UCAGCUGGUAC |
| | | 22547524- | CUGGUAC | ACACGGCAGUUU | ACGGCAGUUUUAGAmGmCmU |
| | | 22547544 | ACGGCA | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCACGAAAGGGCACCGA |
| | | | 413) | CGUUAUCACGAA | GUCGGmU*mG*mC*mU |
| | | | | AGGGCACCGAGU | (SEQ ID NO: 464) |
| | | | | CGGUGCU (SEQ | |
| | | | | ID NO: 434) | |

*The guide sequence disclosed in this Table may be unmodified, modified with the exemplary modification pattern shown in the Table, or modified with a different modification pattern disclosed herein or available in the art.

Throughout this application, the terms “mA,” “mC,” “mU,” or “mG” may be used to denote a nucleotide that has been modified with 2′-O-Me. Throughout this application, the terms A*, C*, U*, or G* may be used to denote a nucleotide that is linked to the next (e.g., 3′) nucleotide with a phosphorothioate (PS) bond.

In some embodiments, the guide RNA comprises a guide sequence selected from any one of SEQ ID NOs: 301-319, 371, 372, 401-407, and 411-413. In some embodiments, the guide RNA comprises a guide sequence that is at least 17, 18, 19, 20, 21, 22, 23, or 24 contiguous nucleotides of a sequence selected from SEQ ID NOs: 301-319, 371, 372, 401-407, and 411-413. In some embodiments, the guide RNA comprises a guide sequence that is at least 95%, 90%, 85%, 80%, 75%, or 70% identical to a sequence selected from SEQ ID NOs: 301-319, 371, 372, 401-407, and 411-413. In some embodiments, the guide RNA comprises a guide sequence that is at least 95% identical to a sequence selected from SEQ ID NOs: 301-319, 371, 372, 401-407, and 411-413.

In some embodiments, the guide RNA comprises a guide sequence selected from any one of SEQ ID NOs: 301-319, 371, 372, 401-407, and 411-413.

In some embodiments, the guide RNA comprises a guide sequence that comprises at least 10 contiguous nucleotides±10 nucleotides of a genomic coordinate listed in Tables 2A, 2B, 3A, and 3B. As used herein, at least 10 contiguous nucleotides±10 nucleotides of a genomic coordinate means, for example, at least 10 contiguous nucleotides within the genomic coordinates wherein the genomic coordinates include 10 nucleotides in the 5′ direction and 10 nucleotides in the 3′ direction from the ranges listed in Tables 2A, 2B, 3A, and 3B. For example, a guide RNA may comprise 10 contiguous nucleotides within the genomic coordinates chr6: 29942891-29942915, chr6: 29942609-29942633, chr6: 29942864-29942884, chr6: 31355222-31355246, chr6: 31355221-31355245, chr6: 31355205-31355229, chr14: 22547524-22547544, chr16: 10907504-10907528, chr16: 10906643-10906667, chr16: 10906853-10906873, chr3: 30674205-30674229, chr3: 30671674-30671698, chr3: 30674167-30674191, chr3: 30671941-30671961, chr3: 30671739-30671759, chr3: 30644885-30644905, chr3: 30671618-30671638, chr3: 30671983-30672003, chr3: 30672094-30672114, chr3: 30672177-30672197, chr3: 30674198-30674218, chr19: 6590121-6590145, chr19: 6586268-6586292, chr19: 6590998-6591018, chr19: 6590991-6591011, chr19: 6590875-6590895, chr19: 6586396-6586416, chr19: 6586388-6586408, chr19: 6586379-6586399, chr19: 6586369-6586389, or chr19: 6586120-6586140, including the boundary nucleotides of these ranges. In some embodiments, the guide RNA comprises a guide sequence that is at least 17, 18, 19, or 20 contiguous nucleotides of a sequence that comprises 10 contiguous nucleotides±10 nucleotides within the genomic coordinates chr6: 29942891-29942915, chr6: 29942609-29942633, chr6: 29942864-29942884, chr6: 31355222-31355246, chr6: 31355221-31355245, chr6: 31355205-31355229, chr14: 22547524-22547544, chr16: 10907504-10907528, chr16: 10906643-10906667, chr16: 10906853-10906873, chr3: 30674205-30674229, chr3: 30671674-30671698, chr3: 30674167-30674191, chr3: 30671941-30671961, chr3: 30671739-30671759, chr3: 30644885-30644905, chr3: 30671618-30671638, chr3: 30671983-30672003, chr3: 30672094-30672114, chr3: 30672177-30672197, chr3: 30674198-30674218, chr19: 6590121-6590145, chr19: 6586268-6586292, chr19: 6590998-6591018, chr19: 6590991-6591011, chr19: 6590875-6590895, chr19: 6586396-6586416, chr19: 6586388-6586408, chr19: 6586379-6586399, chr19: 6586369-6586389, or chr19: 6586120-6586140, or a guide sequence that is complementary to at least 17, 18, 19, 20, 21, 22, 23, or 24 contiguous nucleotides of a sequence that comprises 10 contiguous nucleotides+10 nucleotides within the genomic coordinates chr6: 29942891-29942915, chr6: 29942609-29942633, chr6: 29942864-29942884, chr6: 31355222-31355246, chr6: 31355221-31355245, chr6: 31355205-31355229, chr14: 22547524-22547544, chr16: 10907504-10907528, chr16: 10906643-10906667, chr16: 10906853-10906873, chr3: 30674205-30674229, chr3: 30671674-30671698, chr3: 30674167-30674191, chr3: 30671941-30671961, chr3: 30671739-30671759, chr3: 30644885-30644905, chr3: 30671618-30671638, chr3: 30671983-30672003, chr3: 30672094-30672114, chr3: 30672177-30672197, chr3: 30674198-30674218, chr19: 6590121-6590145, chr19: 6586268-6586292, chr19: 6590998-6591018, chr19: 6590991-6591011, chr19: 6590875-6590895, chr19: 6586396-6586416, chr19: 6586388-6586408, chr19: 6586379-6586399, chr19: 6586369-6586389, or chr19: 6586120-6586140. In some embodiments, the guide RNA comprises a guide sequence that is at least 95%, 90%, or 85% identical to a sequence that is 17, 18, 19, or 20 contiguous nucleotides of a sequence that comprises 10 contiguous nucleotides+10 nucleotides within the genomic coordinates chr6: 29942891-29942915, chr6: 29942609-29942633, chr6: 29942864-29942884, chr6: 31355222-31355246, chr6: 31355221-31355245, chr6: 31355205-31355229, chr14: 22547524-22547544, chr16: 10907504-10907528, chr16: 10906643-10906667, chr16: 10906853-10906873, chr3: 30674205-30674229, chr3: 30671674-30671698, chr3: 30674167-30674191, chr3: 30671941-30671961, chr3: 30671739-30671759, chr3: 30644885-30644905, chr3: 30671618-30671638, chr3: 30671983-30672003, chr3: 30672094-30672114, chr3: 30672177-30672197, chr3: 30674198-30674218, chr19: 6590121-6590145, chr19: 6586268-6586292, chr19: 6590998-6591018, chr19: 6590991-6591011, chr19: 6590875-6590895, chr19: 6586396-6586416, chr19: 6586388-6586408, chr19: 6586379-6586399, chr19: 6586369-6586389, or chr19: 6586120-6586140, or a guide sequence that is complementary to at least 17, 18, 19, 20, 21, 22, 23, or 24 contiguous nucleotides of a sequence that comprises 10 contiguous nucleotides±10 nucleotides within the genomic coordinates chr6: 29942891-29942915, chr6: 29942609-29942633, chr6: 29942864-29942884, chr6: 31355222-31355246, chr6: 31355221-31355245, chr6: 31355205-31355229, chr14: 22547524-22547544, chr16: 10907504-10907528, chr16: 10906643-10906667, chr16: 10906853-10906873, chr3: 30674205-30674229, chr3: 30671674-30671698, chr3: 30674167-30674191, chr3: 30671941-30671961, chr3: 30671739-30671759, chr3: 30644885-30644905, chr3: 30671618-30671638, chr3: 30671983-30672003, chr3: 30672094-30672114, chr3: 30672177-30672197, chr3: 30674198-30674218, chr19: 6590121-6590145, chr19: 6586268-6586292, chr19: 6590998-6591018, chr19: 6590991-6591011, chr19: 6590875-6590895, chr19: 6586396-6586416, chr19: 6586388-6586408, chr19: 6586379-6586399, chr19: 6586369-6586389, or chr19: 6586120-6586140.

In some embodiments, the guide RNA comprises a guide sequence that comprises at least 15 contiguous nucleotides±10 nucleotides within the genomic coordinates chr6: 29942891-29942915, chr6: 29942609-29942633, chr6: 29942864-29942884, chr6: 31355222-31355246, chr6: 31355221-31355245, chr6: 31355205-31355229, chr14: 22547524-22547544, chr16: 10907504-10907528, chr16: 10906643-10906667, chr16: 10906853-10906873, chr3: 30674205-30674229, chr3: 30671674-30671698, chr3: 30674167-30674191, chr3: 30671941-30671961, chr3: 30671739-30671759, chr3: 30644885-30644905, chr3: 30671618-30671638, chr3: 30671983-30672003, chr3: 30672094-30672114, chr3: 30672177-30672197, chr3: 30674198-30674218, chr19: 6590121-6590145, chr19: 6586268-6586292, chr19: 6590998-6591018, chr19: 6590991-6591011, chr19: 6590875-6590895, chr19: 6586396-6586416, chr19: 6586388-6586408, chr19: 6586379-6586399, chr19: 6586369-6586389, or chr19: 6586120-6586140. In some embodiments, the guide RNA comprises a guide sequence that comprises at least 20 contiguous nucleotides±10 nucleotides within the genomic coordinates chr6: 29942891-29942915, chr6: 29942609-29942633, chr6: 29942864-29942884, chr6: 31355222-31355246, chr6: 31355221-31355245, chr6: 31355205-31355229, chr14: 22547524-22547544, chr16: 10907504-10907528, chr16: 10906643-10906667, chr16: 10906853-10906873, chr3: 30674205-30674229, chr3: 30671674-30671698, chr3: 30674167-30674191, chr3: 30671941-30671961, chr3: 30671739-30671759, chr3: 30644885-30644905, chr3: 30671618-30671638, chr3: 30671983-30672003, chr3: 30672094-30672114, chr3: 30672177-30672197, chr3: 30674198-30674218, chr19: 6590121-6590145, chr19: 6586268-6586292, chr19: 6590998-6591018, chr19: 6590991-6591011, chr19: 6590875-6590895, chr19: 6586396-6586416, chr19: 6586388-6586408, chr19: 6586379-6586399, chr19: 6586369-6586389, or chr19: 6586120-6586140.

In some embodiments, wherein the guide RNA is an HLA-A guide RNA, the HLA-A guide RNA comprises SEQ ID NO: 445-448 or 462 or a sequence having at least 90%, 93%, 95%, 96%, 97%, 98%, or 99% identity to SEQ ID NO: 445-448 or 462.

In some embodiments, wherein the guide RNA is an HLA-B guide RNA, the HLA-B guide RNA comprises SEQ ID NO: 451-454 or a sequence having at least 90%, 93%, 95%, 96%, 97%, 98%, or 99% identity to SEQ ID NO: 451-454.

In some embodiments, wherein the guide RNA is a TRAC guide RNA, the TRAC guide RNA comprises SEQ ID NO: 463 or a sequence having at least 90%, 93%, 95%, 96%, 97%, 98%, or 99% identity to SEQ ID NO: 463.

In some embodiments, wherein the guide RNA is a CIITA guide RNA, the CIITA guide RNA comprises SEQ ID NO: 441-444 or 461 or a sequence having at least 90%, 93%, 95%, 96%, 97%, 98%, or 99% identity to SEQ ID NO: 441-444 or 461.

In some embodiments, wherein the guide RNA is a TGFBR2 guide RNA, the TGFBR2 guide RNA comprises SEQ ID NO: 341-352, 391, or 392 or a sequence having at least 90%, 93%, 95%, 96%, 97%, 98%, or 99% identity to SEQ ID NO: 341-352, 391, or 392.

In some embodiments, wherein the guide RNA is a CD70 guide RNA, the CD70 guide RNA comprises SEQ ID NO: 353-366 or a sequence having at least 90%, 93%, 95%, 96%, 97%, 98%, or 99% identity to SEQ ID NO: 353-366.

In some embodiments, the guide RNA is a single guide RNA (sgRNA) comprising a sequence of any one of the sgRNA sequences listed in Tables 2A, 2B, 3A, and 3B, or a sequence having at least 90%, 93%, 95%, 96%, 97%, 98%, or 99% identity to any one of the sgRNA sequences listed in Tables 2A, 2B, 3A, and 3B.

In some embodiments, the compositions provided herein further comprise a HLA-A guide RNA comprising a guide sequence having at least 90%, 93%, 95%, 96%, 97%, 98%, 99% or 100% identity to SEQ ID NO: 403, a HLA-B guide RNA comprising a guide sequence having at least 90%, 93%, 95%, 96%, 97%, 98%, 99% or 100% identity to SEQ ID NO: 406, a CIITA guide RNA comprising a guide sequence having at least 90%, 93%, 95%, 96%, 97%, 98%, 99% or 100% identity to SEQ ID NO: 402, a TGFBR2 guide RNA comprising a guide sequence having at least 90%, 93%, 95%, 96%, 97%, 98%, 99% or 100% identity to SEQ ID NO: 301, a CD70 guide RNA comprising a guide sequence having at least 90%, 93%, 95%, 96%, 97%, 98%, 99% or 100% identity to SEQ ID NO: 310, and a TRAC guide RNA comprising a guide sequence having at least 90%, 93%, 95%, 96%, 97%, 98%, 99% or 100% identity to SEQ ID NO: 413.

In some embodiments, the compositions provided herein further comprise a HLA-A guide RNA comprising a sequence at least 90%, 93%, 95%, 96%, 97%, 98%, 99% or 100% identity to SEQ ID NO: 446, a HLA-B guide RNA comprising a sequence at least 90%, 93%, 95%, 96%, 97%, 98%, 99% or 100% identity to SEQ ID NO: 452, a CIITA guide RNA comprising a sequence at least 90%, 93%, 95%, 96%, 97%, 98%, 99% or 100% identity to SEQ ID NO: 444, a TGFBR2 guide RNA comprising a sequence at least 90%, 93%, 95%, 96%, 97%, 98%, 99% or 100% identity to SEQ ID NO: 342, and a CD70 guide RNA comprising a sequence at least 90%, 93%, 95%, 96%, 97%, 98%, 99% or 100% identity to SEQ ID NO: 354, and a TRAC guide RNA comprising a sequence at least 90%, 93%, 95%, 96%, 97%, 98%, 99% or 100% identity to SEQ ID NO: 464.

Additional embodiments of guide RNAs are provided herein, including e.g., exemplary modifications to the guide RNA.

### 3. Exemplary Genetic Modifications

In some embodiments, the methods and compositions disclosed herein further comprise genetically modifying at least one nucleotide of the HLA-A, HLA-B, TRAC, CIITA, TGFBR2, or CD70 gene in a cell comprising an anti-CD70 CAR. In some embodiments, the genetic modification to HLA-A, HLA-B, TRAC, CIITA, TGFBR2, or CD70 reduces or eliminates the expression of HLA-A, HLA-B, TRAC, MHC class II, TGFBR2, or CD70 protein on the surface of the genetically modified cell (or engineered cell). Genetic modifications encompass the population of modifications that results from contact with a genomic editing system (e.g., the population of edits that result from Cas9 and an HLA-A, HLA-B, TRAC, CIITA, TGFBR2, or CD70 guide RNA, or the population of edits that result from BC22 and an HLA-A, HLA-B, TRAC, CIITA, TGFBR2, or CD70 guide RNA). Methods and compositions for genetic modification of the HLA-A gene are provided in WO2022140586A2, PCT/US2023/068498, and PCT/US2023/068499, the entire contents of which is incorporated herein by reference. Such methods and compositions for genetic modification of HLA-A, HLA-B, CIITA, or TRAC gene, or for reduced or eliminated surface expression of one or more of MHC class II protein, MHC-I protein, TRAC are further described in e.g., International Publication Nos. WO 2020/081613, WO 2022/125982, WO 2022/140586, and WO 2022/140587, and International Application Nos. PCT/US2023/068498 and PCT/US2023/068499, the contents of each of which are hereby incorporated in their entireties. For example, further detailed description of the guide RNAs for reducing or eliminating the expression of TRBC and/or TRAC proteins and for genetic modifications of TRBC and/or TRAC are provided in International Publication No. WO 2020/081613, the entire contents of which are incorporated herein by reference. For example, further detailed description of the guide RNAs for reducing or eliminating the expression of HLA-A and/or CIITA proteins and for genetic modifications of HLA-A and/or CIITA are provided in International Publication No. WO 2022/125982, the entire contents of which are incorporated herein by reference. For example, further detailed description of the guide RNAs for reducing or eliminating the expression of HLA-A protein and for genetic modifications of HLA-A are provided in International Publication No. WO 2022/140586, the entire contents of which are incorporated herein by reference. For example, further detailed description of the guide RNAs for reducing or eliminating the expression of HLA-A and/or CIITA proteins and for genetic modifications of HLA-A and/or CIITA are provided in International Publication No. WO 2022/140587, the entire contents of which are incorporated herein by reference. For example, further detailed description of the guide RNAs for reducing or eliminating the expression of HLA-A and/or HLA-B proteins and for genetic modifications of HLA-A and/or HLA-B are provided in International Application No. PCT/US2023/068498, the entire contents of which are incorporated herein by reference. For example, further detailed description of the guide RNAs for reducing or eliminating the expression of HLA-A, TRAC, TRBC, and/or CIITA proteins and for genetic modifications of HLA-A, TRAC, TRBC, and/or CIITA are provided in International Application No. PCT/US2023/068499, the entire contents of which are incorporated herein by reference.

The following embodiments are directed to the genetic modification in the HLA-A gene:

In some embodiments, the genetic modification comprises at least one nucleotide within the genomic coordinates (a) chr6: 29942864-29942884; and (b) chr6: 29942891-29942915 and chr6: 29942609-29942633.

In some embodiments, the genetic modification comprises an indel, a C to T substitution, or an A to G substitution within the genomic coordinates chosen from: (a) chr6: 29942864-29942884; and (b) chr6: 29942891-29942915 and chr6: 29942609-29942633.

In some embodiments, the modification to HLA-A comprises any one or more of an insertion, deletion, substitution, or deamination of at least one nucleotide in a target sequence.

In some embodiments, the modification to HLA-A comprises an insertion of 1, 2, 3, 4 or 5 or more nucleotides in a target sequence. In some embodiments, the modification to HLA-A comprises a deletion of 1, 2, 3, 4 or 5 or more nucleotides in a target sequence. In other embodiments, the modification to HLA-A comprises an insertion of 1, 2, 3, 4, 5, 6, 7, 8, 9, 10, 15, 20 or 25 or more nucleotides in a target sequence. In other embodiments, the modification to HLA-A comprises a deletion of 1, 2, 3, 4, 5, 6, 7, 8, 9, 10, 15, 20 or 25 or more nucleotides in a target sequence. In some embodiments, the modification to HLA-A comprises an indel, which is generally defined in the art as an insertion or deletion of less than 1000 base pairs (bp). In some embodiments, the modification to HLA-A comprises an indel which results in a frameshift mutation in a target sequence. In some embodiments, the modification to HLA-A comprises a substitution of 1, 2, 3, 4, 5, 6, 7, 8, 9, 10, 15, 20 or 25 or more nucleotides in a target sequence. In some embodiments, the modification to HLA-A comprises one or more of an insertion, deletion, or substitution of nucleotides resulting from the incorporation of a template nucleic acid. In some embodiments, the modification to HLA-A comprises an insertion of a donor nucleic acid in a target sequence. In some embodiments, the modification to HLA-A is not transient.

In some embodiments, the methods and compositions disclosed herein modify the HLA-A gene in a cell using an RNA-guided DNA binding agent (e.g., a Cas enzyme). In some embodiments, the RNA-guided DNA binding agent is Cas9. In some embodiments, the RNA-guided DNA binding agent cuts within the HLA-A gene, wherein the HLA-A guide RNA targets an HLA-A genomic target sequence comprising at least 10 contiguous nucleotides within the genomic coordinates chosen from: (a) chr6: 29942864-29942884; and (b) chr6: 29942891-29942915 and chr6: 29942609-29942633.

In some embodiments, the genetic modification to HLA-A results in utilization of an out-of-frame stop codon. In some embodiments, the genetic modification to HLA-A results in exon skipping during splicing. In some embodiments, the genetic modification to HLA-A results in reduced HLA-A protein expression by the cell. In some embodiments, the modification to the HLA-A results in reduced or eliminated HLA-A protein expression on the surface of the cell.

In some embodiments, HLA-A expression on the surface of a cell is reduced as a result of the genetic modification to HLA-A. In some embodiments, HLA-A expression on the surface of a cell is absent as a result of the genetic modification to HLA-A.

The following embodiments are directed to the genetic modification in the HLA-B gene:

In some embodiments, the genetic modification comprises at least one nucleotide within the genomic coordinateschr6: 31355222-31355246, chr6: 31355221-31355245, or chr6: 31355205-31355229.

In some embodiments, the genetic modification comprises an indel, a C to T substitution, or an A to G substitution within the genomic coordinates chosen from: chr6: 31355222-31355246, chr6: 31355221-31355245, and chr6: 31355205-31355229.

In some embodiments, the modification to HLA-B comprises any one or more of an insertion, deletion, substitution, or deamination of at least one nucleotide in a target sequence. In some embodiments, the modification to HLA-B comprises an insertion of 1, 2, 3, 4 or 5 or more nucleotides in a target sequence. In some embodiments, the modification to HLA-B comprises a deletion of 1, 2, 3, 4 or 5 or more nucleotides in a target sequence. In other embodiments, the modification to HLA-B comprises an insertion of 1, 2, 3, 4, 5, 6, 7, 8, 9, 10, 15, 20 or 25 or more nucleotides in a target sequence. In other embodiments, the modification to HLA-B comprises a deletion of 1, 2, 3, 4, 5, 6, 7, 8, 9, 10, 15, 20 or 25 or more nucleotides in a target sequence. In some embodiments, the modification to HLA-B comprises an indel, which is generally defined in the art as an insertion or deletion of less than 1000 base pairs (bp). In some embodiments, the modification to HLA-B comprises an indel which results in a frameshift mutation in a target sequence. In some embodiments, the modification to HLA-B comprises a substitution of 1, 2, 3, 4, 5, 6, 7, 8, 9, 10, 15, 20 or 25 or more nucleotides in a target sequence. In some embodiments, the modification to HLA-B comprises one or more of an insertion, deletion, or substitution of nucleotides resulting from the incorporation of a template nucleic acid. In some embodiments, the modification to HLA-B comprises an insertion of a donor nucleic acid in a target sequence. In some embodiments, the modification to HLA-B is not transient.

In some embodiments, the methods and compositions disclosed herein modify the HLA-B gene in a cell using an RNA-guided DNA binding agent (e.g., a Cas enzyme). In some embodiments, the RNA-guided DNA binding agent is Cas9. In some embodiments, the RNA-guided DNA binding agent cuts within the HLA-B gene, wherein the HLA-B guide RNA targets an HLA-B genomic target sequence comprising at least 10 contiguous nucleotides within the genomic coordinates chosen from: chr6: 31355222-31355246, chr6: 31355221-31355245, and chr6: 31355205-31355229.

In some embodiments, the genetic modification to HLA-B results in utilization of an out-of-frame stop codon. In some embodiments, the genetic modification to HLA-B results in exon skipping during splicing. In some embodiments, the genetic modification to HLA-B results in reduced HLA-B protein expression by the cell. In some embodiments, the modification to the HLA-B results in reduced or eliminated HLA-B protein expression on the surface of the cell.

In some embodiments, HLA-B expression on the surface of a cell is reduced as a result of the genetic modification to HLA-B. In some embodiments, HLA-B expression on the surface of a cell is absent as a result of the genetic modification to HLA-B.

The following embodiments are directed to the genetic modification in the TRAC gene:

In some embodiments, the genetic modification comprises at least one nucleotide within the genomic coordinates chr14: 22547524-22547544.

In some embodiments, the genetic modification comprises an indel, a C to T substitution, or an A to G substitution within the genomic coordinates chr14: 22547524-22547544.

In some embodiments, the modification to TRAC comprises any one or more of an insertion, deletion, substitution, or deamination of at least one nucleotide in a target sequence. In some embodiments, the modification to TRAC comprises an insertion of 1, 2, 3, 4 or 5 or more nucleotides in a target sequence. In some embodiments, the modification to TRAC comprises a deletion of 1, 2, 3, 4 or 5 or more nucleotides in a target sequence. In other embodiments, the modification to TRAC comprises an insertion of 1, 2, 3, 4, 5, 6, 7, 8, 9, 10, 15, 20 or 25 or more nucleotides in a target sequence. In other embodiments, the modification to TRAC comprises a deletion of 1, 2, 3, 4, 5, 6, 7, 8, 9, 10, 15, 20 or 25 or more nucleotides in a target sequence. In some embodiments, the modification to TRAC comprises an indel, which is generally defined in the art as an insertion or deletion of less than 1000 base pairs (bp). In some embodiments, the modification to TRAC comprises an indel which results in a frameshift mutation in a target sequence. In some embodiments, the modification to TRAC comprises a substitution of 1, 2, 3, 4, 5, 6, 7, 8, 9, 10, 15, 20 or 25 or more nucleotides in a target sequence. In some embodiments, the modification to TRAC comprises one or more of an insertion, deletion, or substitution of nucleotides resulting from the incorporation of a template nucleic acid. In some embodiments, the modification to TRAC comprises an insertion of a donor nucleic acid in a target sequence. In some embodiments, the modification to TRAC is not transient.

In some embodiments, the methods and compositions disclosed herein modify the TRAC gene in a cell using an RNA-guided DNA binding agent (e.g., a Cas enzyme). In some embodiments, the RNA-guided DNA binding agent is Cas9. In some embodiments, the RNA-guided DNA binding agent cuts within the TRAC gene, wherein the TRAC guide RNA targets a TRAC genomic target sequence comprising at least 10 contiguous nucleotides within the genomic coordinates chr14: 22547524-22547544.

In some embodiments, the genetic modification to TRAC results in utilization of an out-of-frame stop codon. In some embodiments, the genetic modification to TRAC results in exon skipping during splicing. In some embodiments, the genetic modification to TRAC results in reduced TRAC protein expression by the cell. In some embodiments, the modification to the TRAC results in reduced or eliminated TRAC protein expression on the surface of the cell.

In some embodiments, TRAC expression on the surface of a cell is reduced as a result of the genetic modification to TRAC. In some embodiments, TRAC expression on the surface of a cell is absent as a result of the genetic modification to TRAC.

The following embodiments are directed to the genetic modification in the CIITA gene:

In some embodiments, the genetic modification comprises at least one nucleotide within the genomic coordinates (a) chr16: 10906853-10906873; and (b) chr16: 10907504-10907528 or chr16: 10906643-10906667.

In some embodiments, the genetic modification comprises an indel, a C to T substitution, or an A to G substitution within the genomic coordinates chosen from: (a) (a) chr16: 10906853-10906873; and (b) chr16: 10907504-10907528 and chr16: 10906643-10906667.

In some embodiments, the modification to CIITA comprises any one or more of an insertion, deletion, substitution, or deamination of at least one nucleotide in a target sequence. In some embodiments, the modification to CIITA comprises an insertion of 1, 2, 3, 4 or 5 or more nucleotides in a target sequence. In some embodiments, the modification to CIITA comprises a deletion of 1, 2, 3, 4 or 5 or more nucleotides in a target sequence. In other embodiments, the modification to CIITA comprises an insertion of 1, 2, 3, 4, 5, 6, 7, 8, 9, 10, 15, 20 or 25 or more nucleotides in a target sequence. In other embodiments, the modification to CIITA comprises a deletion of 1, 2, 3, 4, 5, 6, 7, 8, 9, 10, 15, 20 or 25 or more nucleotides in a target sequence. In some embodiments, the modification to CIITA comprises an indel, which is generally defined in the art as an insertion or deletion of less than 1000 base pairs (bp). In some embodiments, the modification to CIITA comprises an indel which results in a frameshift mutation in a target sequence. In some embodiments, the modification to CIITA comprises a substitution of 1, 2, 3, 4, 5, 6, 7, 8, 9, 10, 15, 20 or 25 or more nucleotides in a target sequence. In some embodiments, the modification to CIITA comprises one or more of an insertion, deletion, or substitution of nucleotides resulting from the incorporation of a template nucleic acid. In some embodiments, the modification to CIITA comprises an insertion of a donor nucleic acid in a target sequence. In some embodiments, the modification to CIITA is not transient.

In some embodiments, the methods and compositions disclosed herein modify the CIITA gene in a cell using an RNA-guided DNA binding agent (e.g., a Cas enzyme). In some embodiments, the RNA-guided DNA binding agent is Cas9. In some embodiments, the RNA-guided DNA binding agent cuts within the CIITA gene, wherein the CIITA guide RNA targets a CIITA genomic target sequence comprising at least 10 contiguous nucleotides within the genomic coordinates chosen from: (a) chr16: 10906853-10906873; and (b) chr16: 10907504-10907528 and chr16: 10906643-10906667.

In some embodiments, the genetic modification to CIITA results in utilization of an out-of-frame stop codon. In some embodiments, the genetic modification to CIITA results in exon skipping during splicing. In some embodiments, the genetic modification to CIITA results in reduced MHC class II protein expression by the cell. In some embodiments, the modification to the CIITA results in reduced or eliminated MHC class II protein expression on the surface of the cell.

In some embodiments, MHC class II expression on the surface of a cell is reduced as a result of the genetic modification to CIITA. In some embodiments, MHC class II expression on the surface of a cell is absent as a result of the genetic modification to CIITA.

The following embodiments are directed to the genetic modification in the TGFBR2 gene:

In some embodiments, the genetic modification comprises at least one nucleotide within the genomic coordinates (a) chr3: 30671941-30671961, chr3: 30671739-30671759, chr3: 30644885-30644905, chr3: 30671618-30671638, chr3: 30671983-30672003, chr3: 30672094-30672114, chr3: 30672177-30672197, or chr3: 30674198-30674218; and (b) chr3: 30674205-30674229, chr3: 30671674-30671698, or chr3: 30674167-30674191.

In some embodiments, the genetic modification comprises an indel, a C to T substitution, or an A to G substitution within the genomic coordinates chosen from: (a) chr3: 30671941-30671961, chr3: 30671739-30671759, chr3: 30644885-30644905, chr3: 30671618-30671638, chr3: 30671983-30672003, chr3: 30672094-30672114, chr3: 30672177-30672197, and chr3: 30674198-30674218; and (b) chr3: 30674205-30674229, chr3: 30671674-30671698, and chr3: 30674167-30674191.

In some embodiments, the modification to TGFBR2 comprises any one or more of an insertion, deletion, substitution, or deamination of at least one nucleotide in a target sequence. In some embodiments, the modification to TGFBR2 comprises an insertion of 1, 2, 3, 4 or 5 or more nucleotides in a target sequence. In some embodiments, the modification to TGFBR2 comprises a deletion of 1, 2, 3, 4 or 5 or more nucleotides in a target sequence. In other embodiments, the modification to TGFBR2 comprises an insertion of 1, 2, 3, 4, 5, 6, 7, 8, 9, 10, 15, 20 or 25 or more nucleotides in a target sequence. In other embodiments, the modification to TGFBR2 comprises a deletion of 1, 2, 3, 4, 5, 6, 7, 8, 9, 10, 15, 20 or 25 or more nucleotides in a target sequence. In some embodiments, the modification to TGFBR2 comprises an indel, which is generally defined in the art as an insertion or deletion of less than 1000 base pairs (bp). In some embodiments, the modification to TGFBR2 comprises an indel which results in a frameshift mutation in a target sequence. In some embodiments, the modification to TGFBR2 comprises a substitution of 1, 2, 3, 4, 5, 6, 7, 8, 9, 10, 15, 20 or 25 or more nucleotides in a target sequence. In some embodiments, the modification to TGFBR2 comprises one or more of an insertion, deletion, or substitution of nucleotides resulting from the incorporation of a template nucleic acid. In some embodiments, the modification to TGFBR2 comprises an insertion of a donor nucleic acid in a target sequence. In some embodiments, the modification to TGFBR2 is not transient.

In some embodiments, the methods and compositions disclosed herein modify the TGFBR2 gene in a cell using an RNA-guided DNA binding agent (e.g., a Cas enzyme). In some embodiments, the RNA-guided DNA binding agent is Cas9. In some embodiments, the RNA-guided DNA binding agent cuts within the TGFBR2 gene, wherein the TGFBR2 guide RNA targets a TGFBR2 genomic target sequence comprising at least 10 contiguous nucleotides within the genomic coordinates chosen from: (a) chr3: 30671941-30671961, chr3: 30671739-30671759, chr3: 30644885-30644905, chr3: 30671618-30671638, chr3: 30671983-30672003, chr3: 30672094-30672114, chr3: 30672177-30672197, and chr3: 30674198-30674218; and (b) chr3: 30674205-30674229, chr3: 30671674-30671698, and chr3: 30674167-30674191.

In some embodiments, the genetic modification to TGFBR2 results in utilization of an out-of-frame stop codon. In some embodiments, the genetic modification to TGFBR2 results in exon skipping during splicing. In some embodiments, the genetic modification to TGFBR2 results in reduced TGFBR2 protein expression by the cell. In some embodiments, the modification to the TGFBR2 results in reduced or eliminated TGFBR2 protein expression on the surface of the cell.

In some embodiments, TGFBR2 expression on the surface of a cell is reduced as a result of the genetic modification to TGFBR2. In some embodiments, TGFBR2 expression on the surface of a cell is absent as a result of the genetic modification to TGFBR2.

The following embodiments are directed to the genetic modification in the CD70 gene:

In some embodiments, the genetic modification comprises at least one nucleotide within the genomic coordinates chosen from: (a) chr19: 6590998-6591018, chr19: 6590991-6591011, chr19: 6590875-6590895, chr19: 6586396-6586416, chr19: 6586388-6586408, chr19: 6586379-6586399, chr19: 6586369-6586389, and chr19: 6586120-6586140; and (b) chr19: 6590121-6590145 and chr19: 6586268-6586292.

In some embodiments, the genetic modification comprises an indel, a C to T substitution, or an A to G substitution within the genomic coordinates chosen from: (a) chr19: 6590998-6591018, chr19: 6590991-6591011, chr19: 6590875-6590895, 6586389, and chr19: 6586120-6586140; and (b) chr19: 6590121-6590145 and chr19: 6586268-6586292.

In some embodiments, the modification to CD70 comprises any one or more of an insertion, deletion, substitution, or deamination of at least one nucleotide in a target sequence. In some embodiments, the modification to CD70 comprises an insertion of 1, 2, 3, 4 or 5 or more nucleotides in a target sequence. In some embodiments, the modification to CD70 comprises a deletion of 1, 2, 3, 4 or 5 or more nucleotides in a target sequence. In other embodiments, the modification to CD70 comprises an insertion of 1, 2, 3, 4, 5, 6, 7, 8, 9, 10, 15, 20 or 25 or more nucleotides in a target sequence. In other embodiments, the modification to CD70 comprises a deletion of 1, 2, 3, 4, 5, 6, 7, 8, 9, 10, 15, 20 or 25 or more nucleotides in a target sequence. In some embodiments, the modification to CD70 comprises an indel, which is generally defined in the art as an insertion or deletion of less than 1000 base pairs (bp). In some embodiments, the modification to CD70 comprises an indel which results in a frameshift mutation in a target sequence. In some embodiments, the modification to CD70 comprises a substitution of 1, 2, 3, 4, 5, 6, 7, 8, 9, 10, 15, 20 or 25 or more nucleotides in a target sequence. In some embodiments, the modification to CD70 comprises one or more of an insertion, deletion, or substitution of nucleotides resulting from the incorporation of a template nucleic acid. In some embodiments, the modification to CD70 comprises an insertion of a donor nucleic acid in a target sequence. In some embodiments, the modification to CD70 is not transient.

In some embodiments, the methods and compositions disclosed herein modify the CD70 gene in a cell using an RNA-guided DNA binding agent (e.g., a Cas enzyme). In some embodiments, the RNA-guided DNA binding agent is Cas9. In some embodiments, the RNA-guided DNA binding agent cuts within the CD70 gene, wherein the CD70 guide RNA targets a CD70 genomic target sequence comprising at least 10 contiguous nucleotides within the genomic coordinates chosen from: (a) chr19: 6590998-6591018, chr19: 6590991-6591011, chr19: 6590875-6590895, chr19: 6586396-6586416, chr19: 6586388-6586408, chr19: 6586379-6586399, chr19: 6586369-6586389, and chr19: 6586120-6586140; and (b) chr19: 6590121-6590145 and chr19: 6586268-6586292.

In some embodiments, the genetic modification to CD70 results in utilization of an out-of-frame stop codon. In some embodiments, the genetic modification to CD70 results in exon skipping during splicing. In some embodiments, the genetic modification to CD70 results in reduced CD70 protein expression by the cell. In some embodiments, the modification to the CD70 results in reduced or eliminated CD70 protein expression on the surface of the cell.

In some embodiments, CD70 expression on the surface of a cell is reduced as a result of the genetic modification to CD70. In some embodiments, CD70 expression on the surface of a cell is absent as a result of the genetic modification to CD70.

### IV. Exemplary Genome Editing Systems

Various suitable gene editing systems may be used to make the engineered cells disclosed herein, including but not limited to the CRISPR/Cas system; zinc finger nuclease (ZFN) system; and the transcription activator-like effector nuclease (TALEN) system. Generally, the gene editing systems involve the use of engineered cleavage systems to induce a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA to guide specific cleavage or nicking of a target DNA sequence. Further, targeted nucleases are being developed based on the Argonaute system (e.g., from T. thermophilus, known as ‘TtAgo’, see Swarts et al (2014) Nature 507 (7491): 258-261), which also may have the potential for uses in gene editing and gene therapy.

In some embodiments, the gene editing system is a TALEN system. Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to a desired DNA sequence, to promote DNA cleavage at specific locations (see, e.g., Boch, 2011, Nature Biotech). The restriction enzymes can be introduced into cells, for use in gene editing or for gene editing in situ, a technique known as gene editing with engineered nucleases. Such methods and compositions for use therein are known in the art. See, e.g., WO2019147805, WO2014040370, WO2018073393, the contents of which are hereby incorporated in their entireties.

In some embodiments, the gene editing system is a zinc-finger system. Zinc-finger nucleases (ZFNs) are artificial restriction enzymes generated by fusing a zinc finger DNA-binding domain to a DNA-cleavage domain. Zinc finger domains can be engineered to target specific desired DNA sequences to enables zinc-finger nucleases to target unique sequences within complex genomes. The non-specific cleavage domain from the type IIs restriction endonuclease FokI is typically used as the cleavage domain in ZFNs. Cleavage is repaired by endogenous DNA repair machinery, allowing ZFN to precisely alter the genomes of higher organisms. Such methods and compositions for use therein are known in the art. See, e.g., WO2011091324, the contents of which are hereby incorporated in their entireties.

In some embodiments, the gene editing system is a CRISPR/Cas system, including e.g., a CRISPR guide RNA comprising a guide sequence and a RNA-guided DNA binding agent, and described further herein. In some embodiments the gene editing system comprises a base editor comprising a deaminase and an RNA-guided nickase. In some embodiments the gene editing system comprises a base editor comprising a cytidine deaminase and an RNA-guided nickase. In some embodiments, the gene editing system comprises a DNA polymerase. Further description of the gene editing system methods and compositions for use therein are known in the art. See e.g., WO2019/067910, WO2021/188840A1, WO2019/051097, and PCT/US2021/062922 filed Dec. 10, 2021, and U.S. Provisional Application No. 63/275,425 filed Nov. 3, 2021, the contents of each of which are hereby incorporated in their entireties. Exemplary nucleotide and polypeptide sequences for the gene editing system disclosed herein are provided below in Table 10. Methods for identifying alternate nucleotide sequences encoding polypeptide sequences provided herein, including alternate naturally occurring variants, are known in the art. Sequences with at least 75%, 80%, 85%, 90%, 95%, 96%, 97%, 98%, or 99% identity to any of the nucleic acid sequences, or nucleic acid sequences encoding the amino acid sequences provided herein are also contemplated.

### V. CRISPR Guide RNA

Provided herein are guide sequences useful for modifying a target sequence, e.g., using a guide RNA comprising a disclosed guide sequence with an RNA-guided DNA binding agent (e.g., a CRISPR/Cas system).

In some aspects, provided herein is a guide RNA comprising: A. a guide sequence comprising a sequence at least 80%, 85%, preferably 90%, or 95% identical to or complementary to at least 20 contiguous nucleotides of any one of the guide sequences of Tables 3A-3B.

In some embodiments, the guide RNAs provided herein further comprise one or more of: A. a shortened hairpin 1 region, or a substituted and optionally shortened hairpin 1 region, wherein 1, at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks a. any one or two of H1-5 through H1-8, b. one, two, or three of the following pairs of nucleotides: H1-1 and H1-12, H1-2 and H1-11, H1-3 and H1-10, and H1-4 and H1-9, or c. 1-8 nucleotides of hairpin 1 region; or 2. the shortened hairpin 1 region lacks 4-8 nucleotides, preferably 4-6 nucleotides; and a. one or more of positions H1-1, H1-2, or H1-3 is deleted or substituted relative to Exemplary SpyCas9 sgRNA-1 (SEQ ID NO: 601) or b. one or more of positions H1-6 through H1-10 is substituted relative to Exemplary SpyCas9 sgRNA-1 (SEQ ID NO: 601); or 3. the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 601); or B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 601); or C. a substitution relative to Exemplary SpyCas9 sgRNA-1 (SEQ ID NO: 601) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or D. an Exemplary SpyCas9 sgRNA-1 (SEQ ID NO: 601) with an upper stem region, wherein the upper stem modification comprises a modification to any one or more of US1-US12 in the upper stem region.

In some embodiments, the guide RNA lacks 6 nucleotides in shortened hairpin 1.

In some embodiments, the guide RNA lacks 8 nucleotides in shortened hairpin 1.

In some embodiments, H-1 and H-3 are deleted.

In some embodiments, the guide RNA further comprises a 3′ tail.

In some embodiments, the 3′ tail is 1-4 nucleotides in length, optionally 1 nucleotide in length.

In some embodiments, the guide RNA comprises an upper stem region comprising a modification to any one or more of US1-US12 in the upper stem region.

In some embodiments, the guide RNAs described herein comprise a nucleotide sequence selected from the sequences in Table 3A.

In some embodiments, the guide RNA comprises a modified nucleotide sequence selected from the modified Spy guide scaffold sequences in Table 5A, wherein the modified nucleotide sequence is 3′ of the guide sequence.

In some embodiments, the guide RNAs described herein are modified according to the pattern of a nucleotide sequence selected from the modified Spy guide RNA sequences in Table 5A-5B.

In some embodiments, the guide comprises a nucleotide sequence selected from the unmodified Spy guide RNA Sequences in Table 4B, wherein the N20's are collectively a guide sequence described herein.

In some embodiments, each nucleotide of the unmodified Spy guide RNA Sequences in Table 4B is any natural or non-natural nucleotide.

In some embodiments, the guide RNA is modified according to a pattern selected from the modification patterns in Table 5B, wherein the (mN*) 3N17 refers to the guide sequence described herein in which the first three nucleotides comprises a 2′-O-Me modification and a phosphorothioate linkage.

In some embodiments, the guide RNAs described herein comprise a sequence or modification pattern set forth in Table 4A-5B.

Guide sequences targeted to sites adjacent to an appropriate PAM, e.g., a Spy Cas9 PAM, e.g., may further comprise additional nucleotides, which can be referred to as a scaffold sequence or a conserved portion, to form a crRNA or a crRNA joined to a trRNA to form a sgRNA e.g., with the following exemplary nucleotide sequence following the guide sequence at its 3′ end (Table 4A). The #mer refers to the length of the crRNA or the sgRNA when a 20 nucleotide guide sequence is included 5′ to the scaffold sequence provided in Table 4A.

In some aspects, provided is a guide RNA (gRNA) comprising a guide region and a conserved region, wherein: A. the guide region comprises a nucleic acid sequence comprising a sequence at least 80%, 85%, preferably 90%, or 95% identical to or complementary to 24 contiguous nucleotides of any one of the guide sequences of Tables 2A-2B.

In some embodiments, the conserved region comprises one or more of: (a) a shortened repeat/anti-repeat region, wherein the shortened repeat/anti-repeat region lacks 2-24 nucleotides relative to SEQ ID NO: 700, wherein (i) one or more of nucleotides 37-48 and 53-64 is deleted relative to SEQ ID NO: 700 and optionally one or more of nucleotides 37-64 is substituted relative to SEQ ID NO: 700; and (ii) nucleotide 36 is linked to nucleotide 65 by at least 2 nucleotides; or (b) a shortened hairpin 1 region, wherein the shortened hairpin 1 lacks 2-10 nucleotides, optionally 2-8 nucleotides relative to SEQ ID NO: 700 wherein (i) one or more of nucleotides 82-86 and 91-95 is deleted relative to SEQ ID NO: 700 and optionally one or more of positions 82-96 is substituted relative to SEQ ID NO: 700; and (ii) nucleotide 81 is linked to nucleotide 96 by at least 4 nucleotides; or (c) a shortened hairpin 2 region, wherein the shortened hairpin 2 lacks 2-18 nucleotides, optionally 2-16 nucleotides relative to SEQ ID NO: 700, wherein (i) one or more of nucleotides 113-121 and 126-134 is deleted relative to SEQ ID NO: 700 and optionally one or more of nucleotides 113-134 is substituted relative to SEQ ID NO: 700; and (ii) nucleotide 112 is linked to nucleotide 135 by at least 4 nucleotides; wherein one or both nucleotides 144-145 are optionally deleted relative to SEQ ID NO: 700; optionally, wherein at least 10 nucleotides are modified nucleotides.

In some embodiments, the conserved region comprises a nucleotide sequence selected from Table 6A-7B.

In some embodiments, the guide RNA comprises at least one end modification.

In some embodiments, the modification comprises a 5′ end modification.

In some embodiments, the modification comprises a 3′ end modification.

In some embodiments, the guide RNA comprises a modification in a hairpin region.

In some embodiments, the modification in a hairpin region is also an end modification.

In some embodiments, the modification comprises a 2′-O-methyl(2′-O-Me) modified nucleotide.

In some embodiments, the modification comprises a phosphorothioate (PS) bond between nucleotides.

In some embodiments, the modification comprises a 2′-O-methyl(2′-O-Me) modified nucleotide with a phosphorothioate (PS) bond to a 3′ adjacent nucleotide.

In some embodiments, the modification comprises a 2′-fluor (2′F) modified nucleotide.

In some embodiments, the 5′ end modification comprises a 2′-O-methyl(2′-O-Me) modified nucleotide with a phosphorothioate (PS) bond to a 3′ adjacent nucleotide at nucleotides 1-3 of the 5′ end of the guide sequence.

In some embodiments, the conserved region comprises a modified nucleotide sequence selected from the modified conserved region Nme guide RNA motifs in Table 7A, and wherein the conserved region is 3′ of the guide region.

In some embodiments, the guide RNA comprises a nucleotide sequence selected from any one of the guide sequences of Tables 2A-2B.

In some embodiments, each nucleotide is any natural or non-natural nucleotide.

In some embodiments, the guide RNA is modified according to a pattern selected from SEQ ID NOs: 710-732, wherein the N's are collectively the guide sequence described herein, wherein N, A, C, G, and U are ribonucleotides (2′-OH), wherein “m” indicates a 2′-O-Me modification, “f” indicates a 2′-fluoro modification, and a “*” indicates a phosphorothioate linkage between nucleotides.

In some aspects, provided herein is a composition comprising a guide RNA described herein.

TABLE 4A
Exemplary Unmodified Spy Scaffold Sequences

| #mer | Unmodified nucleotide sequence | SEQ ID NO |
|---|---|---|
| 42 | GUUUUAGAGCUAUGCUGUUUUG | 599 |
| 100 | GUUUUAGAGCUAGAAAUAGCAAGUUAAAAUAAG | 600 |
| | GCUAGUCCGUUAUCAACUUGAAAAAGUGGCACCG | |
| | AGUCGGUGCUUUU | |
| 96 | GUUUUAGAGCUAGAAAUAGCAAGUUAAAAUAAG | 601 |
| | GCUAGUCCGUUAUCAACUUGAAAAAGUGGCACCG | |
| | AGUCGGUGC | |
| 97 | GUUUUAGAGCUAGAAAUAGCAAGUUAAAAUAAG | 602 |
| | GCUAGUCCGUUAUCAACUUGAAAAAGUGGCACCG | |
| | AGUCGGUGCU | |
| 88 | GUUUUAGAGCUAGAAAUAGCAAGUUAAAAUAAG | 603 |
| | GCUAGUCCGUUAUCAACUUGGCACCGAGUCGGUG | |
| | C | |
| 88 | GUUUUAGAGCUAGAAAUAGCAAGUUAAAAUAAG | 604 |
| | GCUAGUCCGUUAUCAAAAUGGCACCGAGUCGGUG | |
| | C | |
| 88 | GGUUUUAGAGCUAGAAAUAGCAAGUUAAAAUAA | 605 |
| | GGCUAGUCCGUUAUCAAAAUGGCACCGAGUCGGU | |
| | GC | |
| 90 | GUUUUAGAGCUAGAAAUAGCAAGUUAAAAUAAG | 606 |
| | GCUAGUCCGUUAUCACGAAAGGGCACCGAGUCGG | |
| | UGC | |
| 91 | GUUUUAGAGCUAGAAAUAGCAAGUUAAAAUAAG | 607 |
| | GCUAGUCCGUUAUCACGAAAGGGCACCGAGUCGG | |
| | UGCU | |

In some embodiments, the guide RNA comprises a nucleotide sequence selected from the unmodified Spy guide RNA Sequences in Table 4B, wherein the N₂₀'s are collectively any one of the guide sequences of Tables 3A-3B. In some embodiments, each nucleotide of the unmodified Spy guide RNA Sequences in Table 4B is any natural or non-natural nucleotide.

TABLE 4B
Exemplary Unmodified Spy Guide RNA Sequences

| #mer | Unmodified nucleotide sequence | SEQ ID NO |
|---|---|---|
| 100 | (N)20GUUUUAGAGCUAGAAAUAGCAAGUUAAAAU | 610 |
| | AAGGCUAGUCCGUUAUCAACUUGAAAAAGUGGC | |
| | ACCGAGUCGGUGCUUUU | |
| 96 | (N)20GUUUUAGAGCUAGAAAUAGCAAGUUAAAAU | 611 |
| | AAGGCUAGUCCGUUAUCAACUUGAAAAAGUGGC | |
| | ACCGAGUCGGUGC | |
| 97 | (N)20GUUUUAGAGCUAGAAAUAGCAAGUUAAAAU | 612 |
| | AAGGCUAGUCCGUUAUCAACUUGAAAAAGUGGC | |
| | ACCGAGUCGGUGCU | |
| 88 | (N)20GUUUUAGAGCUAGAAAUAGCAAGUUAAAAU | 613 |
| | AAGGCUAGUCCGUUAUCAACUUGGCACCGAGUCG | |
| | GUGC | |
| 88 | (N)20GUUUUAGAGCUAGAAAUAGCAAGUUAAAAU | 614 |
| | AAGGCUAGUCCGUUAUCAAAAUGGCACCGAGUCG | |
| | GUGC | |
| 88 | (N)20GGUUUUAGAGCUAGAAAUAGCAAGUUAAAA | 615 |
| | UAAGGCUAGUCCGUUAUCAAAAUGGCACCGAGUC | |
| | GGUGC | |
| 90 | (N)20GUUUUAGAGCUAGAAAUAGCAAGUUAAAAU | 616 |
| | AAGGCUAGUCCGUUAUCACGAAAGGGCACCGAGU | |
| | CGGUGC | |
| 91 | (N)20GUUUUAGAGCUAGAAAUAGCAAGUUAAAAU | 617 |
| | AAGGCUAGUCCGUUAUCACGAAAGGGCACCGAGU | |
| | CGGUGCU | |

Wherein the Ns collectively are a guide sequence provided herein.

In the case of a sgRNA, the guide sequences may be integrated into the following modified guide scaffold motifs (Table 5A). The #mer refers to the length of the sgRNA when a 20 nucleotide guide sequence, either a modified or unmodified sequence, is included 5′ to the scaffold sequence provided in Table 5A:

TABLE 5A
Exemplary Modified Spy Guide Scaffold Sequences

| #mer | Modified sequence | SEQ ID NO |
|---|---|---|
| 100 | GUUUUAGAmGmCmUmAmGmAmAmAmUmAmGm | 620 |
| | CAAGUUAAAAUAAGGCUAGUCCGUUAUCAmAm | |
| | CmUmUmGmAmAmAmAmAmGmUmGmGmCmAmC | |
| | mCmGmAmGmUmCmGmGmUmGmCmU*mU*mU*m | |
| | U | |
| 96 | GUUUUAGAmGmCmUmAmGmAmAmAmUmAmGm | 630 |
| | CAAGUUAAAAUAAGGCUAGUCCGUUAUCAmAm | |
| | CmUmUmGmAmAmAmAmAmGmUmGmGmCmAmC | |
| | mCmGmAmGmUmCmGmGmUmGmC | |
| 97 | GUUUUAGAmGmCmUmAmGmAmAmAmUmAmGm | 631 |
| | CAAGUUAAAAUAAGGCUAGUCCGUUAUCAmAm | |
| | CmUmUmGmAmAmAmAmAmGmUmGmGmCmAmC | |
| | mCmGmAmGmUmCmGmGmUmGmCmU | |
| 88 | GUUUUAGAmGmCmUmAmGmAmAmAmUmAmGm | 632 |
| | CAAGUUAAAAUAAGGCUAGUCCGUUAUCAACU | |
| | UGGCACCGAGUCGG*mU*mG*mC | |
| 88 | GUUUUAGAmGmCmUmAmGmAmAmAmUmAmGm | 634 |
| | CAAGUUAAAAUAAGGCUAGUCCGUUAUCAAAA | |
| | UGGCACCGAGUCGG*mU*mG*mC | |
| 88 | GUUUUAGAmGmCmUmAmGmAmAmAmUmAmGm | 635 |
| | CAAGUUAAAAUAAGGCUAGUCCGUUAUCAmAm | |
| | AmAmUmGmGmCmAmCmCmGmAmGmUmCmGmG | |
| | *mU*mG*mC | |
| 90 | GUUUUAGAmGmCmUmAmGmAmAmAmUmAmGm | 636 |
| | CAAGUUAAAAUAAGGCUAGUCCGUUAUCACGA | |
| | AAGGGCACCGAGUCGG*mU*mG*mC | |
| 90 | GUUUUAGAmGmCmUmAmGmAmAmAmUmAmGm | 637 |
| | CAAGUUAAAAUAAGGCUAGUCCGUUAUCAmCm | |
| | GmAmAmAmGmGmGmCmAmCmCmGmAmGmUmC | |
| | mGmG*mU*mG*mC | |
| 91 | GUUUUAGAmGmCmUmAmGmAmAmAmUmAmGm | 638 |
| | CAAGUUAAAAUAAGGCUAGUCCGUUAUCACGA | |
| | AAGGGCACCGAGUCGGU*mG*mC*mU | |
| 91 | GUUUUAGAmGmCmUmAmGmAmAmAmUmAmGm | 639 |
| | CAAGUUAAAAUAAGGCUAGUCCGUUAUCACGA | |
| | AAGGGCACCGAGUCGGmUmGmC*mU | |
| 91 | GUUUfUAGmAmGmCmUmAmGmAmAmAmUmAmG | 640 |
| | mCmAmAGUfUmAfAmAfAmUAmAmGmGmCmUmA | |
| | GUmCmCGUfUAmUmCAmCmGmAmAmAmGmGmG | |
| | mCmAmCmCmGmAmGmUmCmGmGmU*mG*mC*m | |
| | U | |
| 91 | GUUUUAGAmGmCmUmAmGmAmAmAmUmAmGm | 641 |
| | CAAGUUAAAAUAAGGCUAGUCCGUUAUCACGA | |
| | AAGGGCACCGAGUCGGmU*mG*mC*mU | |

wherein “m” indicates a 2′-O-Me modification, “f” indicates a 2′-fluoro modification, a indicates a phosphorothioate linkage between nucleotides, and no modification in the context of a modified sequence indicates an RNA (2′-OH) and phosphodiesterase linkage.

A guide sequence is present on the 5′ end of the conserved portion of the guide RNA. In certain embodiments, the guide sequence is 20-25, preferably 22-24 nucleotides in length. In certain embodiments, the guide sequence comprises one or more chemical modifications, for example modifications at one or more of nucleotides 1, 2, and 3, optionally all of nucleotides 1, 2, and 3 at the 5′ end of the guide RNA. In certain embodiments, the modification comprises a 2′-O-Me modification.

In certain embodiments, the guide sequence is a chemically modified sequence. In certain embodiments, the chemically modified guide sequence is (mN*)₃(N)₁₃₋₁₇. In certain embodiments, the guide sequence is (mN*)₃(N)₁₇, i.e., mN*mN*mN*NNNNNNNNNNNNNNNNN. In certain embodiments, each N of the (N)₁₃₋₁₇ or the (N)₁₇ is unmodified. In certain embodiments, the each N in the (N)₁₃₋₁₇ or the (N)₁₇ is independently modified, e.g., independently modified with a 2′-O-methyl modification.

In some embodiments, the sgRNA comprises any of the modification patterns shown herein, where N is any natural or non-natural nucleotide, and wherein the totality of the N's comprise any one of the guide sequences disclosed in Tables 3A-3B. In some embodiments, the modified sgRNA comprises a sequence shown in Table 5B.

TABLE 5B
Exemplary Modified Spy Guide RNA Sequences

| | SEQ ID | |
|---|---|---|
| #mer | NO | Modified sequence |
| 100 | 658 | (mN*)3N17GUUUUAGAmGmCmUmAmGmAmAmAmUmAmGmCAA |
| | | GUUAAAAUAAGGCUAGUCCGUUAUCAmAmCmUmUmGmAmA |
| | | mAmAmAmGmUmGmGmCmAmCmCmGmAmGmUmCmGmGmUm |
| | | GmCmU*mU*mU*mU |
| 96 | 659 | (mN*)3N17GUUUUAGAmGmCmUmAmGmAmAmAmUmAmGmCA |
| | | AGUUAAAAUAAGGCUAGUCCGUUAUCAmAmCmUmUmGmAm |
| | | AmAmAmAmGmUmGmGmCmAmCmCmGmAmGmUmCmGmGmU |
| | | mGmC |
| 97 | 660 | (mN*)3N17GUUUUAGAmGmCmUmAmGmAmAmAmUmAmGmCA |
| | | AGUUAAAAUAAGGCUAGUCCGUUAUCAmAmCmUmUmGmAm |
| | | AmAmAmAmGmUmGmGmCmAmCmCmGmAmGmUmCmGmGmU |
| | | mGmCmU |
| 88 | 661 | (mN*)3N17GUUUUAGAmGmCmUmAmGmAmAmAmUmAmGmCA |
| | | AGUUAAAAUAAGGCUAGUCCGUUAUCAACUUGGCACCGAGU |
| | | CGG*mU*mG*mC |
| 88 | 662 | (mN*)3N17GUUUUAGAmGmCmUmAmGmAmAmAmUmAmGmCA |
| | | AGUUAAAAUAAGGCUAGUCCGUUAUCAAAAUGGCACCGAGU |
| | | CGG*mU*mG*mC |
| 88 | 663 | (mN*)3N17GUUUUAGAmGmCmUmAmGmAmAmAmUmAmGmCA |
| | | AGUUAAAAUAAGGCUAGUCCGUUAUCAmAmAmAmUmGmGm |
| | | CmAmCmCmGmAmGmUmCmGmG*mU*mG*mC |
| 90 | 664 | (mN*)3N17GUUUUAGAmGmCmUmAmGmAmAmAmUmAmGmCA |
| | | AGUUAAAAUAAGGCUAGUCCGUUAUCACGAAAGGGCACCGA |
| | | GUCGG*mU*mG*mC |
| 90 | 665 | (mN*)3N17GUUUUAGAmGmCmUmAmGmAmAmAmUmAmGmCA |
| | | AGUUAAAAUAAGGCUAGUCCGUUAUCAmCmGmAmAmAmGm |
| | | GmGmCmAmCmCmGmAmGmUmCmGmG*mU*mG*mC |
| 91 | 666 | (mN*)3N17GUUUUAGAmGmCmUmAmGmAmAmAmUmAmGmCA |
| | | AGUUAAAAUAAGGCUAGUCCGUUAUCACGAAAGGGCACCGA |
| | | GUCGGU*mG*mC*mU |
| 91 | 667 | (mN*)3N17GUUUUAGAmGmCmUmAmGmAmAmAmUmAmGmCA |
| | | AGUUAAAAUAAGGCUAGUCCGUUAUCACGAAAGGGCACCGA |
| | | GUCGGmUmGmC*mU |
| 91 | 668 | (mN*)3N17GUUUfUAGmAmGmCmUmAmGmAmAmAmUmAmGm |
| | | CmAmAGUfUmAfAmAfAmUAmAmGmGmCmUmAGUmCmCGUfU |
| | | AmUmCAmCmGmAmAmAmGmGmGmCmAmCmCmGmAmGmUm |
| | | CmGmGmU*mG*mC*mU |
| 91 | 669 | (mN*)3N17GUUUUAGAmGmCmUmAmGmAmAmAmUmAmGmCA |
| | | AGUUAAAAUAAGGCUAGUCCGUUAUCACGAAAGGGCACCGA |
| | | GUCGGmU*mG*mC*mU |

wherein “m” indicates a 2′-O-Me modification, “f” indicates a 2′-fluoro modification, and a ‘indicates a phosphorothioate linkage between nucleotides, and no modification in the context of a modified sequence indicates an RNA (2’-OH), wherein the totality of N's comprise a guide sequence comprising a sequence at least 85%, preferably 90% or 95% identical to or complementary to at least 17, 18, 19, or 20 contiguous nucleotides of any of the guide sequences disclosed herein in Tables 3A-3B, where the N's are replaced with any of the guide sequences disclosed herein in Tables 3A-3B. In certain embodiments, when the totality of N's comprise a guide sequence, within N17, each N of the N17 may be independently modified, e.g., modified with a 2′-OMe modification.

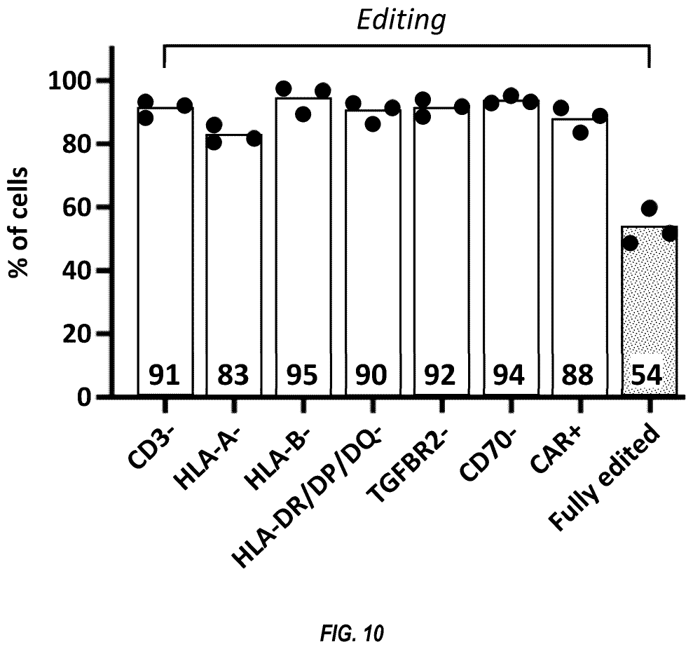

In the case of a sgRNA, the guide sequences may further comprise a SpyCas9 sgRNA scaffold sequence. An example of a SpyCas9 sgRNA scaffold sequence is shown in the Table 8A below (SEQ ID NO: 601: GUUUUAGAGCUAGAAAUAGCAAGUUAAAAU AAGGCUAGUC CGUUAUCAACUUGAAAAAGU GGCACCGAGUCGGUGC-“Exemplary SpyCas9 sgRNA-1”), included at the 3′ end of the guide sequence, and provided with the domains as shown in the table below. LS is lower stem. B is bulge. US is upper stem. H1 and H2 are hairpin 1 and hairpin 2, respectively. Collectively H1 and H2 are referred to as the hairpin region. A model of the structure containing both a guide sequence and a scaffold sequence is provided in FIG. 10A of WO2019237069, which is incorporated herein by reference.

The nucleotide sequence of Exemplary SpyCas9 sgRNA-1 may serve as a template sequence for specific chemical modifications, sequence substitutions and truncations.

In certain embodiments, the gRNA is an sgRNA or a dgRNA, for example, and it optionally comprises a chemical modification. In some embodiments, the modified sgRNA comprises a guide sequence and a SpyCas9 sgRNA sequence, e.g., Exemplary SpyCas9 sgRNA-1. A gRNA, such as an sgRNA, may include modifications on the 5′ end of the guide sequence or on the 3′ end of the SpyCas9 sgRNA sequence, such as, e.g., Exemplary SpyCas9 sgRNA-1 at one or more of the terminal nucleotides, e.g., at 1, 2, 3, or 4 of the nucleotides at the 3′ end or at the 5′ end. In certain embodiments, the modified nucleotide is selected from a 2′-O-methyl(2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, or an inverted abasic modified nucleotide; or a combination thereof. In certain embodiments, the modified nucleotide includes a 2′-OMe modified nucleotide. In certain embodiments, the modified nucleotide includes a PS linkage. In certain embodiments, the modified nucleotide includes a 2′-OMe modified nucleotide and a PS linkage.

In certain embodiments, using SEQ ID NO: 601 GUUUUAGAGCUAGAAAUAGCAAGUUAAAAUAAGGCUAGUCCGUUAUCAAC UUGAAAAAGUGGCACCGAGUCGGUGC “Exemplary SpyCas9 sgRNA-1,” see WO2019237069, the contents of which are incorporated herein by reference). The portions of the Exemplary SpyCas9 sgRNA-1 and position numbering scheme are set forth in Table 8A below.

As an example, the Exemplary SpyCas9 sgRNA-1 further includes one or more of:
A. a shortened hairpin 1 region, or a substituted and optionally shortened hairpin 1 region, wherein
   1. at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks
      a. any one or two of H1-5 through H1-8,
      b. one, two, or three of the following pairs of nucleotides: H1-1 and H1-12, H1-2 and H1-11, H1-3 and H1-10, and H1-4 and H1-9, or
      c. 1-8 nucleotides of hairpin 1 region; or
   2. the shortened hairpin 1 region lacks 4-8 nucleotides, preferably 4-6 nucleotides; and
      a. one or more of positions H1-1, H1-2, or H1-3 is deleted or substituted relative to Exemplary SpyCas9 sgRNA-1 (SEQ ID NO: 601) or
      b. one or more of positions H1-6 through H1-10 is substituted relative to Exemplary SpyCas9 sgRNA-1 (SEQ ID NO: 601); or
      3. the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 601); or
B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 601); or
C. a substitution relative to Exemplary SpyCas9 sgRNA-1 (SEQ ID NO: 601) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
D. an Exemplary SpyCas9 sgRNA-1 (SEQ ID NO: 601) with an upper stem region, wherein the upper stem modification comprises a modification to any one or more of US1-US12 in the upper stem region, wherein
   1. the modified nucleotide is optionally selected from a 2′-O-methyl(2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, and a combination thereof; or
   2. the modified nucleotide optionally includes a 2′-OMe modified nucleotide.

Guide sequences targeted to sites adjacent to an appropriate PAM, e.g., an NmeCas9 PAM, may further comprise additional nucleotides to form a crRNA or a crRNA joined to a trRNA to form a sgRNA e.g., with the exemplary nucleotide sequence following the guide sequence at its 3′ end as provided in Tables 6A-7B. The portions of the Exemplary NmeCas9 sgRNA and position numbering scheme, including both a guide sequence and a scaffold sequence, are set forth in Table 8B below.

In certain embodiments, using SEQ ID NO: 700 (“Exemplary NmeCas9 sgRNA-1”), as an example, the Exemplary NmeCas9 sgRNA-1 includes:
A. A guide RNA (gRNA) comprising a guide region and a conserved region, the conserved region comprising one or more of:
   (a) a shortened repeat/anti-repeat region, wherein the shortened repeat/anti-repeat region lacks 2-24 nucleotides, wherein
(i) one or more of nucleotides 37-48 and 53-64 is deleted and optionally one or more of nucleotides 37-64 is substituted relative to SEQ ID NO: 700; and
(ii) nucleotide 36 is linked to nucleotide 65 by at least 2 nucleotides; or
   (b) a shortened hairpin 1 region, wherein the shortened hairpin 1 lacks 2-10, optionally 2-8 nucleotides, wherein
(i) one or more of nucleotides 82-86 and 91-95 is deleted and optionally one or more of positions 82-96 is substituted relative to SEQ ID NO: 700; and
(ii) nucleotide 81 is linked to nucleotide 96 by at least 4 nucleotides; or
   (c) a shortened hairpin 2 region, wherein the shortened hairpin 2 lacks 2-18, optionally 2-16 nucleotides, wherein
(i) one or more of nucleotides 113-121 and 126-134 is deleted and optionally one or more of nucleotides 113-134 is substituted relative to SEQ ID NO: 700; and
(ii) nucleotide 112 is linked to nucleotide 135 by at least 4 nucleotides;
   wherein one or both nucleotides 144-145 are optionally deleted relative to SEQ ID NO: 700;
wherein, optionally, at least 10 nucleotides are modified nucleotides.

Exemplary unmodified conserved nucleotide sequences, also referred to as scaffold sequences, are shown in Table 6A. The #mer refers to the length of the sgRNA when a 24 nucleotide guide sequence is included 5′ to the scaffold sequence provided in Table 6A.

TABLE 6A
Exemplary Unmodified Nme Guide RNA Conserved Region Nucleotide
Sequences

| #mer | Unmodified nucleotide sequence of conserved portion | SEQ ID NO |
|---|---|---|
| 145 | GUUGUAGCUCCCUUUCUCAUUUCGGAAACGAAAUGAGAACCG | 705 |
| | UUGCUACAAUAAGGCCGUCUGAAAAGAUGUGCCGCAACGCUC | |
| | UGCCCCUUAAAGCUUCUGCUUUAAGGGGCAUCGUUUA | |
| 101 | GUUGUAGCUCCCUGAAACCGUUGCUACAAUAAGGCCGUCGAA | 706 |
| | AGAUGUGCCGCAACGCUCUGCCUUCUGGCAUCGUU | |
| 105 | GUUGUAGCUCCCUUCGAAAGACCGUUGCUACAAUAAGGCCGUC | 707 |
| | GAAAGAUGUGCCGCAACGCUCUGCCUUCUGGCAUCGUU | |
| 107 | GUUGUAGCUCCCUGGAAACCCGUUGCUACAAUAAGGCCGUCGA | 708 |
| | AAGAUGUGCCGCAACGCUCUGCCUUCUGGCAUCGUUUAUU | |

In some embodiments, the guide RNA comprises a nucleotide sequence selected from the unmodified Nme guide RNA Sequences in Tables 2A-2B, wherein the N₂₀₋₂₅'s are collectively any one of the guide sequences disclosed in Tables 2A-2B. In some embodiments, each nucleotide of the unmodified Spy guide RNA Sequences in Table 6B is any natural or non-natural nucleotide.

TABLE 6B
Exemplary Unmodified Nme Guide RNA Nucleotide Sequences

| | Unmodified nucleotide sequence | |
|---|---|---|
| #mer | of conserved portion | SEQ ID NO |
| 145 | NNNNNNNNNNNNNNNNNNNNNNNNNGUUGUAGCUC | 700 |
| | CCUUUCUCAUUUCGGAAACGAAAUGAGAACCGUUG | |
| | CUACAAUAAGGCCGUCUGAAAAGAUGUGCCGCAAC | |
| | GCUCUGCCCCUUAAAGCUUCUGCUUUAAGGGGCAU | |
| | CGUUUA | |
| 145 | (N)₂₀₋₂₅ | 701 |
| | GUUGUAGCUCCCUUUCUCAUUUCGGAAACGAAAUG | |
| | AGAACCGUUGCUACAAUAAGGCCGUCUGAAAAGAU | |
| | GUGCCGCAACGCUCUGCCCCUUAAAGCUUCUGCUUU | |
| | AAGGGGCAUCGUUUA | |
| 101 | (N)₂₀₋₂₅ | 702 |
| | GUUGUAGCUCCCUGAAACCGUUGCUACAAUAAGGC | |
| | CGUCGAAAGAUGUGCCGCAACGCUCUGCCUUCUGG | |
| | CAUCGUU | |
| 105 | (N)₂₀₋₂₅ | 703 |
| | GUUGUAGCUCCCUUCGAAAGACCGUUGCUACAAUA | |
| | AGGCCGUCGAAAGAUGUGCCGCAACGCUCUGCCUU | |
| | CUGGCAUCGUU | |
| 107 | (N)₂₀₋₂₅ | 704 |
| | GUUGUAGCUCCCUGGAAACCCGUUGCUACAAUAAG | |
| | GCCGUCGAAAGAUGUGCCGCAACGCUCUGCCUUCU | |
| | GGCAUCGUUUAUU | |
| 105 | (N)₂₀₋₂₅ | 1000 |
| | GUUGUAGCUCCCUGAAACCGUUGCUACAAUAAGGC | |
| | CGUCGAAAGAUGUGCCGCAACGCUCUGCCUUCUGG | |
| | CAUCGUUUAUU | |
| 101 | NNNNNNNNNNNNNNNNNNNNNNNNNGUUGUAGCUC | 1001 |
| | CCUGAAACCGUUGCUACAAUAAGGCCGUCGAAAGA | |
| | UGUGCCGCAACGCUCUGCCUUCUGGCAUCGUU | |

In the case of a sgRNA, modified guide sequences may be integrated into one of the following exemplary modified conserved portion motifs (Table 7A). The #mer refers to the length of the sgRNA when a 24 nucleotide guide sequence, either a modified or unmodified sequence, is included 5′ to the scaffold sequence provided in Table 6A or 7A:

TABLE 7A
Exemplary Modified Nme Guide RNA Conserved Regions

| | | SEQ |
|---|---|---|
| | | ID |
| #mer | Modified nucleotide sequence of conserved portion | NO |
| 101 | mGUUGmUmAmGmCUCCCmUmGmAmAmAmCmCGUUmGmCUAmCAA | 710 |
| | U*AAGmGmCCmGmUmCmGmAmAmAmGmAmUGUGCmCGCmAmAmC | |
| | mGCUCUmGmCCmUmUmCmUGmGCmAmUC*mG*mU*mU | |
| 101 | mGUUGmUmAmGmCUCCCmUmGmAmAmAmCmCGUUmGmCUAmCAA | 712 |
| | U*AAGmGmCCmGmUmCmGmAmAmAmGmAmUGUGCmCGmCAAmCG | |
| | CUCUmGmCCmUmUmCmUGGCAUCG*mU*mU | |
| 101 | mGUUGmUmAmGmCUCCCmUmGmAmAmAmCmCGUUmGmCUAmCAA | 713 |
| | UAAGmGmCCmGmUmCmGmAmAmAmGmAmUGUGCmCGmCAAmCGC | |
| | UCUmGmCCmUmUmCmUGGCAUCG*mU*mU | |
| 101 | mGUUGmUmAmGmCUCCCmUmGmAmAmAmCmCGUUmGmCUAmCAA | 714 |
| | U*AAGmGmCCmGmUmCmGmAmAmAmGmAmUGUGCmCGmCAAmCG | |
| | mCmUmCmUmGmCCmUmUmCmUGGCAUCG*mU*mU | |
| 101 | mGUUGmUmAmGmCUCCCmUmGmAmAmAmCmCGUUmGmCUAmCAA | 715 |
| | UAAGmGmCCmGmUmCmGmAmAmAmGmAmUGUGCmCGmCAAmCGm | |
| | CmUmCmUmGmCCmUmUmCmUGGCAUCG*mU*mU | |
| 105 | mGUUGmUmAmGmCUCCCmUmUmCmGmAmAmAmGmAmCmCGUUmG | 716 |
| | mCUAmCAAU*AAGmGmCCmGmUmCmGmAmAmAmGmAmUGUGCmC | |
| | GmCAAmCGCUCUmGmCCmUmUmCmUGGCAUCG*mU*mU | |
| 105 | mGUUGmUmAmGmCUCCCmUmUmCmGmAmAmAmGmAmCmCGUUmG | 717 |
| | mCUAmCAAUAAGmGmCCmGmUmCmGmAmAmAmGmAmUGUGCmCG | |
| | mCAAmCGCUCUmGmCCmUmUmCmUGGCAUCG*mU*mU | |
| 105 | mGUUGmUmAmGmCUCCCmUmUmCmGmAmAmAmGmAmCmCGUUmG | 718 |
| | mCUAmCAAU*AAGmGmCCmGmUmCmGmAmAmAmGmAmUGUGCmC | |
| | GmCAAmCGmCmUmCmUmGmCCmUmUmCmUGGCAUCG*mU*mU | |
| 105 | mGUUGmUmAmGmCUCCCmUmUmCmGmAmAmAmGmAmCmCGUUmG | 719 |
| | mCUAmCAAUAAGmGmCCmGmUmCmGmAmAmAmGmAmUGUGCmCG | |
| | mCAAmCGmCmUmCmUmGmCCmUmUmCmUGGCAUCG*mU*mU | |

wherein “m” indicates a 2′-O-Me modification, and a “*” indicates a phosphorothioate linkage between nucleotides, and no modification in the context of a modified sequence indicates an RNA (2′-OH) and a phosphorothioate linkage.

A guide sequence is present on the 5′ end of the conserved portion of the guide RNA. In certain embodiments, the guide sequence is 20-25, preferably 22-24 nucleotides in length. In certain embodiments, the guide sequence comprises one or more chemical modifications, for example modifications at one or more of nucleotides 1, 2, and 3, optionally all of nucleotides 1, 2, and 3 at the 5′ end of the guide RNA. In certain embodiments, the modification comprises a 2′-O-Me modification.

In certain embodiments, the modification comprises a 2′-O-Me modification and a phosphorothioate linkage to the 3′ nucleotide, e.g., (mN*)₃(N)₁₇₋₂₂, preferably (mN*)₃(N)₂₁, wherein each of the nucleotides in the (N) 21 portion is independently modified or unmodified.

In certain embodiment, the totality of N's comprise a GUIDE sequence comprising: (A) a sequence at least 80%, 85%, preferably at least 90%, or 95% identical, or 100% identical to or complementary to 24 contiguous nucleotides of . . . a target site provided in Table 2A. For example, where the N's are replaced with any of the guide sequences disclosed herein in Table 2A. In certain embodiments, when the totality of N's comprise a guide sequence, within (N) 20-25, each N of the (N) 20-25 may be independently modified, e.g., modified with a 2′-OMe modification, optionally further with a PS modification, particularly at 1, 2, or 3 terminal nucleotides. In certain embodiments, the (N) 20-25 has the following sequence and modification pattern
mN*mN*mN*mNmNNNmNmNNmNNmNNNNNmNNNNmNNN.

In some embodiments, the sgRNA comprises any of the modification patterns shown herein, where N is any natural or non-natural nucleotide, and wherein the totality of the N's comprise a guide sequence disclosed in Tables 2A-2B. In some embodiments, the modified sgRNA comprises a sequence shown in Table 7B.

TABLE 7B
Exemplary Modified Nme Guide RNA sequences

| | | SEQ |
|---|---|---|
| | | ID |
| #mer | Modified nucleotide sequence | NO |
| 101 | mN*mNNNNNNNNmNNNmNNNNNNNNNNNNmGUUGmUmAmGmCUCCCmUm | 720 |
| | GmAmAmAmCmCGUUmGmCUAmCAAU*AAGmGmCCmGmUmCmGmAmAmAm | |
| | GmAmUGUGCmCGCmAmAmCmGCUCUmGmCCmUmUmCmUGmGCmAmUC*m | |
| | G*mU*mU | |
| 101 | (N) 20-25 | 721 |
| | mGUUGmUmAmGmCUCCCmUmGmAmAmAmCmCGUUmGmCUAmCAAU*AAG | |
| | mGmCCmGmUmCmGmAmAmAmGmAmUGUGCmCGmCAAmCGCUCUmGmCCm | |
| | UmUmCmUGGCAUCG*mU*mU | |
| 101 | (N) 20-25 | 722 |
| | mGUUGmUmAmGmCUCCCmUmGmAmAmAmCmCGUUmGmCUAmCAAUAAGm | |
| | GmCCmGmUmCmGmAmAmAmGmAmUGUGCmCGmCAAmCGCUCUmGmCCmU | |
| | mUmCmUGGCAUCG*mU*mU | |
| 101 | (N) 20-25 | 723 |
| | mGUUGmUmAmGmCUCCCmUmGmAmAmAmCmCGUUmGmCUAmCAAU*AAG | |
| | mGmCCmGmUmCmGmAmAmAmGmAmUGUGCmCGmCAAmCGmCmUmCmUmG | |
| | mCCmUmUmCmUGGCAUCG*mU*mU | |
| 101 | (N) 20-25 | 724 |
| | mGUUGmUmAmGmCUCCCmUmGmAmAmAmCmCGUUmGmCUAmCAAUAAGm | |
| | GmCCmGmUmCmGmAmAmAmGmAmUGUGCmCGmCAAmCGmCmUmCmUmGm | |
| | CCmUmUmCmUGGCAUCG*mU*mU | |
| 105 | (N) 20-25 | 725 |
| | mGUUGmUmAmGmCUCCCmUmUmCmGmAmAmAmGmAmCmCGUUmGmCUAm | |
| | CAAU*AAGmGmCCmGmUmCmGmAmAmAmGmAmUGUGCmCGmCAAmCGCU | |
| | CUmGmCCmUmUmCmUGGCAUCG*mU*mU | |
| 105 | (N) 20-25 | 726 |
| | mGUUGmUmAmGmCUCCCmUmUmCmGmAmAmAmGmAmCmCGUUmGmCUAm | |
| | CAAUAAGmGmCCmGmUmCmGmAmAmAmGmAmUGUGCmCGmCAAmCGCUC | |
| | UmGmCCmUmUmCmUGGCAUCG*mU*mU | |
| 105 | (N) 20-25 | 727 |
| | mGUUGmUmAmGmCUCCCmUmUmCmGmAmAmAmGmAmCmCGUUmGmCUAm | |
| | CAAU*AAGmGmCCmGmUmCmGmAmAmAmGmAmUGUGCmCGmCAAmCGmC | |
| | mUmCmUmGmCCmUmUmCmUGGCAUCG*mU*mU | |
| 105 | (N) 20-25 | 728 |
| | mGUUGmUmAmGmCUCCCmUmUmCmGmAmAmAmGmAmCmCGUUmGmCUAm | |
| | CAAUAAGmGmCCmGmUmCmGmAmAmAmGmAmUGUGCmCGmCAAmCGmCm | |
| | UmCmUmGmCCmUmUmCmUGGCAUCG*mU*mU | |
| 105 | mN*mN*mN*mNmNNNmNmNNmNNmNNNNNmNNNNmNNNmGUUGmUmAmG | 729 |
| | mCUCCCmUmUmCmGmAmAmAmGmAmCmCGUUmGmCUAmCAAU*AAGmGm | |
| | CCmGmUmCmGmAmAmAmGmAmUGUGCmCGmCAAmCGCUCUmGmCCmUmU | |
| | mCmUGGCAUCG*mU*mU | |
| 105 | mN*mN*mN*mNmNNNmNmNNmNNmNNNNNmNNNNmNNNmGUUGmUmAmG | 730 |
| | mCUCCCmUmUmCmGmAmAmAmGmAmCmCGUUmGmCUAmCAAUAAGmGmC | |
| | CmGmUmCmGmAmAmAmGmAmUGUGCmCGmCAAmCGCUCUmGmCCmUmUm | |
| | CmUGGCAUCG*mU*mU | |
| 101 | mN*mN*mN*mNmNNNmNmNNmNNmNNNNNmNNNNmNNNmGUUGmUmAmG | 731 |
| | mCUCCCmUmGmAmAmAmCmCGUUmGmCUAmCAAU*AAGmGmCCmGmUmC | |
| | mGmAmAmAmGmAmUGUGCmCGmCAAmCGCUCUmGmCCmUmUmCmUGGCA | |
| | UCG*mU*mU | |
| 101 | mN*mN*mN*mNmNNNmNmNNmNNmNNNNNmNNNNmNNNmGUUGmUmAmG | 732 |
| | mCUCCCmUmGmAmAmAmCmCGUUmGmCUAmCAAUAAGmGmCCmGmUmCm | |
| | GmAmAmAmGmAmUGUGCmCGmCAAmCGCUCUmGmCCmUmUmCmUGGCAU | |
| | CG*mU*mU | |

wherein “m” indicates a 2′-O-Me modification, and a “*” indicates a phosphorothioate linkage between nucleotides, and no modification in the context of a modified sequence indicates an RNA (2′-OH) and a phosphorothioate linkage.

In certain embodiments, Exemplary SpyCas9 sgRNA-1, Exemplary NmeCas9 sgRNA-1, or an sgRNA, such as an sgRNA comprising an Exemplary SpyCas9 sgRNA-1, further includes a 3′ tail, e.g., a 3′ tail of 1, 2, 3, 4, or more nucleotides. In certain embodiments, the tail includes one or more modified nucleotides. In certain embodiments, the modified nucleotide is selected from a 2′-O-methyl(2′-OMe) modified nucleotide, a 2′-O-(2-methoxyethyl) (2′-O-moe) modified nucleotide, a 2′-fluoro (2′-F) modified nucleotide, a 2′ deoxy (2′H-) modified nucleotide, an abasic nucleotide, a locked nucleic acid (LNA) nucleotide, an unlocked nucleic acid (UNA) nucleotide, a phosphorothioate (PS) linkage between nucleotides, and a terminal inverted abasic modified nucleotide; or is a combination thereof. In certain embodiments, the modified nucleotide includes a 2′-OMe modified nucleotide. In certain embodiments, the modified nucleotide includes a PS linkage between nucleotides. In certain embodiments, the modified nucleotide includes a 2′-OMe modified nucleotide and a PS linkage between nucleotides.

In certain embodiments, the hairpin region includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, and an inverted abasic modified nucleotide; or is a combination thereof. In certain embodiments, the modified nucleotide includes a 2′-OMe modified nucleotide.

In certain embodiments, the upper stem region includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, and an inverted abasic modified nucleotide; or is a combination thereof. In certain embodiments, the modified nucleotide includes a 2′-OMe modified nucleotide.

In certain embodiments, the Exemplary SpyCas9 sgRNA-1 or the Exemplary NmeCas9 sgRNA-1 comprises one or more YA dinucleotides, wherein Y is a pyrimidine, wherein the YA dinucleotide includes a modified nucleotide. In certain embodiments, the modified nucleotide selected from a 2′-O-methyl(2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, and an inverted abasic modified nucleotide; or is a combination thereof. In certain embodiments, the modified nucleotide includes a 2′-OMe modified nucleotide.

In certain embodiments, the Exemplary SpyCas9 sgRNA-1 or the Exemplary NmeCas9 sgRNA-1 comprises one or more YA dinucleotides, wherein Y is a pyrimidine, wherein the YA dinucleotide includes a sequence substituted nucleotide, wherein the pyrimidine is substituted for a purine. In certain embodiments, when the pyrimidine forms a Watson-Crick base pair in the single guide, the Watson-Crick based nucleotide of the sequence substituted pyrimidine nucleotide is substituted to maintain Watson-Crick base pairing.

TABLE 8A
Exemplary spyCas9 sgRNA sgRNA-1(SEQ ID NO: 601)

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|---|---|---|---|---|---|---|---|---|---|---|---|---|---|
| G | U | U | U | U | A | G | A | G | C | U | A | G | A | A | A | U | A | G | C | A | A | G | U | U | A | A | A | A | U |
| LS1-LS6 | | | | | | B1-B2 | | US1-US12 | | | | | | | | | | | | B2-B6 | | | | LS7-LS12 | | | | | |
| 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 | 57 | 58 | 59 | 60 |
| A | A | G | G | C | U | A | G | U | C | C | G | U | U | A | U | C | A | A | C | U | U | G | A | A | A | A | A | G | U |

| Nexus | | | | | | | | | H1-1 through H1-12 | | | | | | |
|---|---|---|---|---|---|---|---|---|---|---|---|---|---|---|---|
| 61 | 62 | 63 | 64 | 65 | 66 | 67 | 68 | 69 | 70 | 71 | 72 | 73 | 74 | 75 | 76 |
| G | G | C | A | C | C | G | A | G | U | C | G | G | U | G | C |

| | |
|---|---|
| N | H2-1 through H2-15 |

TABLE 8B
Exemplary NmeCas9 sgRNA(SEQ ID NO: 700)

| 1-24 | 25 | 26 | 27 | 28 | 29 | 30 | 31 | 32 | 33 |
|---|---|---|---|---|---|---|---|---|---|
| NNNNNNNNNNNNNNNNNNNNNNNN | G | U | U | G | U | A | G | C | U |

| | |
|---|---|
| | Lower stem |
| Guide region | Repeat/Anti-Repeat region |

| 34 | 35 | 36 | 37 | 38 | 39 | 40 | 41 | 42 | 43 | 44 | 45 | 46 | 47 | 48 |
|---|---|---|---|---|---|---|---|---|---|---|---|---|---|---|
| C | C | C | U | U | U | C | U | C | A | U | U | U | C | G |

| | |
|---|---|
| Lower stem | Upper stem |

Repeat/Anti-Repeat region

| 49 | 50 | 51 | 52 | 53 | 54 | 55 | 56 | 57 | 58 | 59 | 60 | 61 | 62 | 63 | 64 | 65 | 66 | 67 | 68 | 69 | 70 | 71 | 72 | 73 | 74 | 75 | 76 | 77 |
|---|---|---|---|---|---|---|---|---|---|---|---|---|---|---|---|---|---|---|---|---|---|---|---|---|---|---|---|---|
| G | A | A | A | C | G | A | A | A | U | G | A | G | A | A | C | C | G | U | U | G | C | U | A | C | A | A | U | A |

| | | |
|---|---|---|
| Loop | Upper stem | Lower stem |

Repeat/Anti-Repeat region

| 78 | 79 | 80 | 81 | 82 | 83 | 84 | 85 | 86 | 87 | 88 | 89 | 90 | 91 | 92 | 93 | 94 |
|---|---|---|---|---|---|---|---|---|---|---|---|---|---|---|---|---|
| A | G | G | C | C | G | U | C | U | G | A | A | A | A | G | A | U |

| | | |
|---|---|---|
| Stem | Loop | Stem (96: unpaired) |

Hairpin 1

| 95 | 96 | 97 | 98 | 99 | 100 | 101 | 102 | 103 | 104 | 105 | 106 | 107 | 108 |
|---|---|---|---|---|---|---|---|---|---|---|---|---|---|
| G | U | G | C | C | G | C | A | A | C | G | C | U | C |

| | | |
|---|---|---|
| Stem (96: unpaired) | Lower stem | Bulge |

| | |
|---|---|
| Hairpin 1 | Hairpin 2 |

| 109 | 110 | 111 | 112 | 113 | 114 | 115 | 116 | 117 | 118 | 119 | 120 | 121 | 122 |
|---|---|---|---|---|---|---|---|---|---|---|---|---|---|
| U | G | C | C | C | C | U | U | A | A | A | G | C | U |

| | |
|---|---|
| Upper Stem | Loop |

Hairpin 2

| 123 | 124 | 125 | 126 | 127 | 128 | 129 | 130 | 131 | 132 | 133 | 134 |
|---|---|---|---|---|---|---|---|---|---|---|---|
| U | C | U | G | C | U | U | U | A | A | G | G |

| | |
|---|---|
| Loop | Upper Stem |

Hairpin 2

| 135 | 136 | 137 | 138 | 139 | 140 | 141 | 142 | 143 | 144 | 145 |
|---|---|---|---|---|---|---|---|---|---|---|
| G | G | C | A | U | C | G | U | U | U | A |

| | | |
|---|---|---|
| Upper Stem | Bulge | Lower Stem |

| | |
|---|---|
| Hairpin 2 | Tail |

Linker Containing gRNAs

In certain embodiments, the gRNA comprises one or more internal linkers. As used herein, “internal linker” describes a non-nucleotide segment joining two nucleotides within a guide RNA. If the gRNA contains a spacer region, the internal linker is located outside of the spacer region (e.g., in the scaffold or conserved region of the gRNA). For Type V guides, it is understood that the last hairpin is the only hairpin in the structure, i.e., the repeat-anti-repeat region. The length of an internal linker may be dependent on, for example, the number of nucleotides replaced by the linker and the position of the linker in the gRNA. Internal linkers and their use in the context of gRNA are provided in WO2022261292.

gRNAs disclosed herein may comprise an internal linker. In general, any internal linker compatible with the function of the gRNA may be used. It may be desirable for the linker to have a degree of flexibility. In some embodiments, the internal linker comprises at least two, three, four, five, six, or more on-pathway single bonds. A bond is on-pathway if it is part of the shortest path of bonds between the two nucleotides whose 5′ and 3′ positions are connected to the linker.

As used herein the length of the internal linker can be defined by its bridging length. The “bridging length” of an internal linker as used herein refers to the distance or number of atoms in the shortest chain of atoms on the pathway from the first atom of the linker (bound to a 3′ substituent, such as an oxygen or phosphate, of the preceding nucleotide to the last atom of the linker (bound to a 5′ substituent, such as an oxygen or phosphate) of the following nucleotide) (e.g., from ~ to # in the structure of Formula (I) described below). Approximate predicted bridging lengths for various linkers are provided in a table below.

Exemplary predicted linker lengths by number of atoms, number of ethylene glycol units, approximate linker length in Angstroms on the assumption that an ethylene glycol monomer is about 3.7 Angstroms, and suitable location for substitution of at least the entire loop portion of a hairpin structure are provided in the table 9A. Substitution of two nucleotides requires a linker length of at least about 11 Angstroms. Substitution of at least 3 nucleotides requires a linker length of at least about 16 Angstroms.

TABLE 9A

| | Number of | | |
|---|---|---|---|
| | Ethylene | Approximate | |
| Number of | Glycol | length in | Suitable location for complete |
| atoms | units | Angstroms | loop substitution |
| 3 | 1 | 3.7 | Repeat-anti-repeat (for both loop |
| | | | and stem when no stem present) |
| 6 | 2 | 7.4 | Repeat-anti-repeat (for both loop |
| | | | and stem when no stem present) |
| 9 | 3 | 11.1 | Repeat-anti-repeat (for both loop |
| | | | and stem when no stem present), |
| | | | Nexus |
| 12 | 4 | 14.8 | Nexus |
| 15 | 5 | 18.5 | Repeat-anti-repeat, hairpin 1, |
| | | | hairpin 2 |
| 18 | 6 | 22.2 | Repeat-anti-repeat, hairpin 1, |
| | | | hairpin 2 |
| 21 | 7 | 25.9 | Repeat-anti-repeat, hairpin 1, |
| | | | hairpin 2 |
| 24 | 8 | 29.6 | Repeat-anti-repeat, hairpin 1, |
| | | | hairpin 2 |
| 27 | 9 | 33.3 | Repeat-anti-repeat, hairpin 1, |
| | | | hairpin 2 |
| 30 | 10 | 37 | Repeat-anti-repeat, hairpin 1, |
| | | | hairpin 2 |

In some embodiments, the internal linker comprises a structure of formula (I):

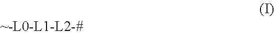

wherein:
~ indicates a bond to a 3′ substituent of the preceding nucleotide;
# indicates a bond to a 5′ substituent of the following nucleotide;
LO is null or C₁₋₃ aliphatic;
L1 is -[E¹-(R¹)]ₘ-, where
each R¹ is independently a C₁₋₅ aliphatic group, optionally substituted with 1 or 2 E²,
each E¹ and E² are independently a hydrogen bond acceptor, or are each independently chosen from cyclic hydrocarbons, and heterocyclic hydrocarbons, and
each m is 1, 2, 3, 4, 5, 6, 7, 8, 9, or 10; and
L2 is null, C₁₋₃ aliphatic, or is a hydrogen bond acceptor.

In some embodiments, L1 comprises one or more —CH₂CH₂O—,
CH₂OCH₂—, or —OCH₂CH₂— units (“ethylene glycol subunits”). In some embodiments, the number of —CH₂CH₂O—, —CH₂OCH₂—, or —OCH₂CH₂— units is in the range of 1, 2, 3, 4, 5, 6, 7, 8, 9 or 10.

In some embodiments, m is 1, 2, 3, 4 or 5. In some embodiments, m is 1, 2, or 3. In some embodiments, m is 6, 7, 8, 9, or 10.

In some embodiments, L0 is null. In some embodiments, L0 is —CH₂— or —CH₂CH₂—.

In some embodiments, L2 is null. In some embodiments, L2 is —O—, —S—, or C₁₋₃ aliphatic. In some embodiments, L2 is —O—. In some embodiments, L2 is —S—. In some embodiments, L2 is —CH₂— or —CH₂CH₂—.

In the tables herein, L1 and L2, are optionally, C9 and C18, respectively as follows:

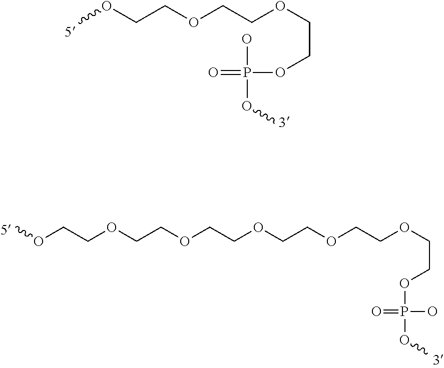

In certain embodiments, the internal linker has a bridging length of about 3-30 atoms, optionally 12-21 atoms, and the linker substitutes for at least 2 nucleotides of the gRNA. In certain embodiments, the internal linker has a bridging length of about 6-18 atoms, optionally about 6-12 atoms, and the linker substitutes for at least 2 nucleotides of the gRNA. In certain embodiments, the internal linker substitutes for 2-12 nucleotides.

In some embodiments, the guide RNA comprises a nucleic acid sequence of SEQ ID NO: 601 or SEQ ID NO: 700, including modifications disclosed elsewhere herein. Table 9B shows various embodiments of the gRNA structures and species with possible number of internal linkers and positions.

TABLE 9B

| gRNA | | # internal | |
|---|---|---|---|
| structures | Type | linkers | Positions of internal linkers |
| Repeat/anti- | Spy | 3 | Repeat/Anti-Repeat region; nexus |
| R; nexus; | | | (within hairpin or replace hairpin), |
| Hp1; Hp2 | | | Hairpin 1 (Hp1) |
| Repeat/anti- | Spy | 2 | Any two of Repeat/Anti-R; nexus |
| R; nexus; | | | (within hairpin or replace hairpin), |
| Hp1; Hp2 | | | Hp1 |
| Repeat/anti- | Spy | 1 | Any of Repeat/Anti-R; nexus |
| R; nexus; | | | (within hairpin or replace hairpin), |
| Hp1; Hp2 | | | Hp1 |
| Repeat/anti- | Nme | 3 | All of repeat/anti-R; Hp1; Hp2 |
| R; Hp1; | | | |
| Hp2 | | | |
| Repeat/anti- | Nme | 2 | Any two of repeat/anti-R; Hp1; |
| R; Hp1; | | | Hp2 |
| Hp2 | | | |
| Repeat/anti- | Nme | 1 | Any one of repeat/anti-R; Hp1; |
| R; Hp1; | | | Hp2 |
| Hp2 | | | |

In certain embodiments, the internal linker is in a repeat-anti-repeat region of the gRNA. In certain embodiments, the internal linker substitutes for at least 4 nucleotides of the repeat-anti-repeat region of the gRNA. In certain embodiments, the internal linker substitutes for the loop in the repeat-anti-repeat region of a Spy Cas9 gRNA, corresponding to nucleotides 13-16 in SEQ ID NO: 601. In certain embodiments, the internal linker substitutes for the loop in the repeat-anti-repeat region of an Nme Cas9 gRNA, corresponding to nucleotides 49-52 in SEQ ID NO: 700.

In certain embodiments, the internal linker substitutes for 2, 3, or 4 nucleotides of the nexus region of the gRNA. In certain embodiments, the internal linker substitutes for the loop in the nexus region of a Spy Cas9 gRNA corresponding to nucleotides 33-36 of SEQ ID NO: 601.

In certain embodiments, the internal linker is in a hairpin region of the gRNA. In certain embodiments, the internal linker substitutes for at least 4 nucleotides of the hairpin region of the gRNA. In certain embodiments, the internal linker substitutes for the loop in the hairpin 1 region of a Spy Cas9 gRNA, corresponding to nucleotides 53-56 in SEQ ID NO: 601. In certain embodiments, the internal linker substitutes for the loop in the hairpin 1 region of an Nme Cas9 gRNA, corresponding to nucleotides 87-90 in SEQ ID NO: 700. In certain embodiments, the internal linker substitutes for at least 4 nucleotides the loop in the hairpin 2 region of an Nme Cas9 gRNA, corresponding to nucleotides 122-125 in SEQ ID NO: 700. In certain embodiments, the internal linker substitutes for the loop in the hairpin 1 region of an Nme Cas9 gRNA, corresponding to nucleotides 87-90 in SEQ ID NO: 700 and for at least 4 nucleotides the loop in the hairpin 2 region of an Nme Cas9 gRNA, corresponding to nucleotides 122-125 in SEQ ID NO: 700.

TABLE 9C
Exemplary SpyCas9 guide RNAs comprising linkers

| SEQ | |
|---|---|
| ID | |
| NO: | gRNA sequence |
| 670 | NNNNNNNNNNNNNNNNNNNNGUUUUAGAGCUA(L1)UAGCAAGUUAAAAUAAGGC(L2)GU |
| | CCGUUAUCAACUU(L1)AAGUGGCACCGAGUCGGUGCUUUU |
| 671 | NNNNNNNNNNNNNNNNNNNNGUUUUAGAGCUA(L1)UAGCAAGUUAAAAUAAGGC(L2)GU |
| | CCGUUAUCAC(L1)GGGCACCGAGUCGGUGC |
| 672 | mN*mN*mN*NNNNNNNNNNNNNNNNNGUUUUAGAmGmCmUmA(L1)mUmAmGmCAAGUU |
| | AAAAUAAGGC(L2)GUCCGUUAUCAC(L1)GGGCACCGAGUCGG*mU*mG*mC |
| 673 | mN*mN*mN*NNNNNNNNNNNNNNNNNGUUUUAGAmGmCmUmA(L1)mUmAmGmCAAGUU |
| | AAAAUAAGGC(L2)GUCCGUUAUCA(L1)GGCACCGAGUCGG*mU*mG*mC |
| 674 | mN*mN*mN*NNNNNNNNNNNNNNNNNGUUUUAGAmGmCmU(L1)mAmGmCAAGUUAAAA |
| | UAAGGC(L2)GUCCGUUAUCAC(L1)GGGCACCGAGUCGG*mU*mG*mC |
| 675 | mN*mN*mN*NNNNNNNNNNNNNNNNNGUUUUAGAmGmCmU(L1)mAmGmCAAGUUAAAA |
| | UAAGGC(L2)GUCCGUUAUCA(L1)GGCACCGAGUCGG*mU*mG*mC |
| 676 | mN*mN*mN*NNNNNNNNNNNNNNNNNGUUUUAGAmGmCmU(L1)mAmGmCAAGUUAAAA |
| | UAAGGC(L2)GUCCGUUAUCA(L1)GGCACCGAGUCGG*mU*mG*mC |
| 677 | mN*mN*mN*NNNNNNNNNNNNNNNNNGUUUUAGAmGmCmU(L1)mAmGmCAAGUUAAAA |
| | UAAGGC(L2)GUCCGUUAUCA(L1)GGCACCGAGUCGG*mU*mG*mC |
| 678 | mN*mN*mN*NNNNNNNNNNNNNNNNNGUUUUAGAmGmCmUmA(L1)mUmAmGmCAAGUU |
| | AAAAUAAGGC(L2)GUCCGUUAUCAC(L1)GGGCACCGAGUCGG*mU*mG*mC |
| 679 | mN*mN*mN*NNNNNNNNNNNNNNNNNGUUUUAGAmGmCmUmA(L1)mUmAmGmCAAGUU |
| | AAAAUAAGGC(L2)GUCCGUUAUCA(L1)GGCACCGAGUCGG*mU*mG*mC |
| 680 | mN*mN*mN*NNNNNNNNNNNNNNNNNGUUUUAGAmGmCmU(L1)mAmGmCAAGUUAAAA |
| | UAAGGC(L2)GUCCGUUAUCAC(L1)GGGCACCGAGUCGG*mU*mG*mC |
| 681 | mN*mN*mN*NNNNNNNNNNNNNNNNNGUUUUAGAmGmCmU(L1)mAmGmCAAGUUAAAA |
| | UAAGGC(L2)GUCCGUUAUCA(L1)GGCACCGAGUCGG*mU*mG*mC |
| 682 | mN*mN*mN*NNNNNNNNNNNNNNNNNGUUUUAGA(dS)AAGUUAAAAUAAGGCUAGUCCG |
| | UUAUCAC(L1)GGGCACCGAGUCGGmU*mG*mC*mU |
| 683 | mN*mN*mN*NNNNNNNNNNNNNNNNNGUUUUAGA(L4)AAGUUAAAAUAAGGCUAGUCCG |
| | UUAUCAC(L1)GGGCACCGAGUCGGmU*mG*mC*mU |
| 684 | mN*mN*mN*NNNNNNNNNNNNNNNNNGUUUUAGA(L3)AAGUUAAAAUAAGGCUAGUCCG |
| | UUAUCAC(L1)GGGCACCGAGUCGGmU*mG*mC*mU |
| 685 | mN*mN*mN*NNNNNNNNNNNNNNNNNGUUUUAGA(L2)AAGUUAAAAUAAGGCUAGUCCG |
| | UUAUCAC(L1)GGGCACCGAGUCGGmU*mG*mC*mU |
| 686 | mN*mN*mN*NNNNNNNNNNNNNNNNNGUUUUAGA(L1)AAGUUAAAAUAAGGCUAGUCCG |
| | UUAUCAC(L1)GGGCACCGAGUCGGmU*mG*mC*mU |
| 687 | mN*mN*mN*NNNNNNNNNNNNNNNNNGUUUUAGAmGmC(L1)mGmCAAGUUAAAAUAAG |
| | GCUAGUCCGUUAUCAC(L1)GGGCACCGAGUCGGmU*mG*mC*mU |
| 688 | mN*mN*mN*NNNNNNNNNNNNNNNNNGUUUUAGAmGmCmUmA(L1)mUmAmGmCAAGUU |
| | AAAAUAAGGCUAGUCCGUUAUCAC(L1)GGGCACCGAGUCGGmU*mG*mC*mU |
| 689 | mN*mN*mN*NNNNNNNNNNNNNNNNNGUUUUAGAmGmCmUmA(L1)mUmAmGmCAAGUU |
| | AAAAUAAGGCUAGUCCGUUAUCAC(L1)GGGCACCGAGUmCmGmGmU*mG*mC*mU |

Nucleotide modifications in modified sequences are indicated in Table 9C as follows: wherein “m” indicates a 2′-O-Me modification, a “*” indicates a phosphorothioate linkage between nucleotides, and within the individually indicated nucleotides, no modification indicates an RNA (2′-OH) with a phosphodiesterase backbone.

TABLE 9D
Exemplary NmeCas9 guide RNAs comprising linkers

| SEQ | |
|---|---|
| ID | |
| NO: | gRNA sequence |
| 770 | (N)₂₀₋₂₅ |
| | GUUGUAGCUCCCUUC(L1)GACCGUUGCUACAAUAAGGCCGUC(L1)GAUGUGC |
| | CGCAACGCUCUGCC(L1)GGCAUCGUU |
| 771 | mN*mN*mN*mNmNNNmNmNNmNNmNNNNNmNNNNmNNNmGUUGmUmAmG |
| | mCUCCCmUmUmC(L1)mGmAmCmCGUUmGmCUAmCAAU*AAGmGmCCmGmU |
| | mC(L1)mGmAmUGUGCmCGmCAAmCGCUCUmGmCC(L1)GGCAUCG*mU*mU |
| 772 | mN*mN*mN*mNmNNNmNmNNmNNmNNNNNmNNNNmNNNmGUUGmUmAmG |
| | mCUCCCmU(L1)mCmCGUUmGmCUAmCAAU*AAGmGmCCmGmUmC(L1)mGmA |
| | mUGUGCmCGmCAAmCGCUCUmGmCC(L1)GGCAUCG*mU*mU |
| 773 | NNNNNNNNNNNNNNNNNNNNNNNNmGUUGmUmAmGmCUCCCmU(L1)mCmC |
| | GUUmGmCUAmCAAU*AAGmGmCCmGmUmC(L1)mGmAmUGUGCmCGmCAAm |
| | CGCUCUmGmCC(L1)GGCAUCG*mU*mU |
| 774 | (N)₂₀₋₂₅ |
| | GUUGmUmAmGmCUCCCmU(L1)mCmCGUUmGmCUAmCAAU*AAGmGmCCmG |
| | mUmC(L1)mGmAmUGUGCmCGmCAAmCGCUCUmGmCC(L1)GGCAUCG*mU*m |
| | U |
| 775 | (N)₂₀₋₂₅ |
| | GUUGmUmAmGmCUCCCmUmUmG(L1)mCmAmCmCGUUmGmCUAmCAAU*AA |
| | GmGmCCmGmUmC(L1)mGmAmUGUGCmCGmCAAmCGCUCUmGmCC(L1)GGCA |
| | UCGmU*mU |
| 776 | N)₂₀₋₂₅ |
| | GUUGmUmAmGmCUCCCmUmUmG(L1)mCmAmCmCGUUmGmCUAmCAAU*AA |
| | GmGmCCmGmUmC(L1)mGmAmUGUGCmCGmCAAmCGCUCUmGmCC(L1)GGCA |
| | UCGmU*mU |
| 777 | N)₂₀₋₂₅ |
| | GUUGmUmAmGmCUCCCmUmG(L1)mCmCmCGUUmGmCUAmCAAU*AAGmGm |
| | CCmGmUmC(L1)mGmAmUGUGCmCGmCAAmCGCUCUmGmCC(L1)GGCAUCGm |
| | U*mU |
| 778 | N)₂₀₋₂₅ |
| | GUUGmUmAmGmCUCCCmG(L1)mCmCGUUmGmCUAmCAAU*AAGmGmCCmG |
| | mUmC(L1)mGmAmUGUGCmCGmCAAmCGCUCUmGmCC(L1)GGCAUCGmU*mU |
| 779 | N)₂₀₋₂₅ |
| | GUUGmUmAmGmCUCCmG(L1)mCGUUmGmCUAmCAAU*AAGmGmCCmGmUm |
| | C(L1)mGmAmUGUGCmCGmCAAmCGCUCUmGmCC(L1)GGCAUCGmU*mU |
| 780 | N)₂₀₋₂₅ |
| | GUUGmUmAmGmCUCC(L1)GUUmGmCUAmCAAU*AAGmGmCCmGmUmC(L1)m |
| | GmAmUGUGCmCGmCAAmCGCUCUmGmCC(L1)GGCAUCGmU*mU |
| 781 | N)₂₀₋₂₅ |
| | GUUGmUmAmGmCUCCCmUmUmG(L1)mCmAmCmCGUUmGmCUAmCAAU*AA |
| | GmGmCCmGmUmCmU(L1)mAmGmAmUGUGCmCGmCAAmCGCUCUmGmCC(L1) |
| | GGCAUCGmU*mU |
| 782 | N)₂₀₋₂₅ |
| | GUUGmUmAmGmCUCCCmUmUmG(L1)mCmAmCmCGUUmGmCUAmCAAU*AA |
| | GmGmCCmGmUmC(L1)mGmAmUGUGCmCGmCAAmCGCUCUmGmCCmU(L1)m |
| | AGGCAUCGmU*mU |

Nucleotide modifications in modified sequences are indicated in Table 9D as follows: wherein “m” indicates a 2′-O-Me modification, a “*” indicates a phosphorothioate linkage between nucleotides, and within the individually indicated nucleotides, no modification indicates an RNA (2′-OH) with a phosphodiesterase backbone. Even in the context of a modified sequence, each nucleotide of (N)20-25 is optionally independently modified. In certain examples, at least the first three nucleotides are modified, e.g., (mN*)3(N)17-22.

In some embodiments, a composition comprising one or more guide RNAs comprising a guide sequence of any one listed in Tables 3A-3B is provided. In some embodiments, a composition comprising one or more guide RNAs comprising a guide sequence of any one in Tables 3A-3B is provided, wherein the nucleotides of SEQ ID: 617 follow the guide sequence at its 3′ end. In some embodiments, the one or more guide RNAs comprising a guide sequence of any one in Tables 3A-3B, wherein the nucleotides of SEQ ID NO: 617 follow the guide sequence at its 3′ end, is modified according to the modification pattern of any one of the sequences shown in Table 5A (e.g., SEQ ID NO: 641). In some embodiments, the one or more guide RNAs comprising a guide sequence of any one in Tables 3A-3B, wherein the nucleotides of SEQ ID NO: 600 follow the guide sequence at its 3′ end, is modified according to the modification pattern of any one of the sequences shown in Table 5B (e.g., SEQ ID NO: 658).

In some embodiments, an sgRNA comprising the guide sequence of any one listed in Tables 3A-3B and any conserved portion of an sgRNA shown in Tables 5A-5B, optionally having a modification pattern of any of an sgRNA shown in Tables 5B, optionally wherein the sgRNA comprises a 5′ and 3′ end modification (if not already shown in the construct of Table 5B) is provided.

In some embodiments, the sgRNA comprises any of the modification patterns shown above in Table 5B, where N is any natural or non-natural nucleotide, and wherein the totality of the N's comprise a guide sequence as described herein in Table 3A. Table 5B does not depict the guide sequence portion of the sgRNA. The modifications remain as shown in Table 5B despite the substitution of N's for the nucleotides of a guide sequence. That is, although the nucleotides of the guide replace the “N's”, the nucleotides are modified as shown in Table 5B.

In some embodiments, a composition comprising one or more guide RNAs comprising a guide sequence of any one in Tables 2A-2B is provided. In some embodiments, a composition comprising one or more guide RNAs comprising a guide sequence of any one in Tables 2A-2B is provided, wherein the nucleotides of SEQ ID: 706 follow the guide sequence at its 3′ end. In some embodiments, the one or more guide RNAs comprising a guide sequence of any one in Tables 2A-2B, wherein the nucleotides of SEQ ID NO: 706 follow the guide sequence at its 3′ end, is modified according to the modification pattern of any one of SEQ ID NOs: 710-719. In some embodiments, the one or more guide RNAs comprising a guide sequence of any one in Tables 2A-2B, wherein the nucleotides of SEQ ID NO: 706 follow the guide sequence at its 3′ end, is modified according to the modification pattern of any one of the sequences shown in Table 7A (e.g., SEQ ID NOs: 712 or 713). In some embodiments, the one or more guide RNAs comprising a guide sequence of any one in Tables 2A-2B, wherein the nucleotides of SEQ ID NO: 706 follow the guide sequence at its 3′ end, is modified according to the modification pattern of SEQ ID NO: 713.

In some embodiments, an sgRNA comprising the guide sequence of any one listed in Tables 2A-2B and any conserved portion of an sgRNA shown in Tables 7A-7B, optionally having a modification pattern of any of an sgRNA shown in Tables 7B, optionally wherein the sgRNA comprises a 5′ and 3′ end modification (if not already shown in the construct of Table 7B) is provided.

In some embodiments, the sgRNA comprises any of the modification patterns shown below in Table 7B, where Nis any natural or non-natural nucleotide, and wherein the totality of the N's comprise a guide sequence as described herein in Table 2A. Table 7B does not depict the guide sequence portion of the sgRNA. The modifications remain as shown in Table 7B despite the substitution of N's for the nucleotides of a guide sequence. That is, although the nucleotides of the guide replace the “N's”, the nucleotides are modified as shown in Table 7B.

In some embodiments, a composition comprising one or more guide RNAs comprising a guide sequence of any one in Tables 2A-2B is provided. In one aspect, a composition comprising one or more gRNAs is provided, comprising a guide sequence that is at least 99%, 98%, 97%, 96%, 95%, 94%, 93%, 92%, 91%, or 90% identical to any of the nucleic acids of any one in Tables 2A-2B.

In some embodiments, a composition comprising one or more guide RNAs comprising a guide sequence of any one in Tables 3A-3B is provided. In one aspect, a composition comprising one or more gRNAs is provided, comprising a guide sequence that is at least 99%, 98%, 97%, 96%, 95%, 94%, 93%, 92%, 91%, or 90% identical to any of the nucleic acids of any one in Tables 3A-3B.

In other embodiments, a composition is provided that comprises at least one, e.g., at least two gRNA's comprising guide sequences selected from any two or more of the guide sequences shown in any one in Tables 2A-2B. In some embodiments, the composition comprises at least two gRNA's that each comprise a guide sequence at least 99%, 98%, 97%, 96%, 95%, 94%, 93%, 92%, 91%, or 90% identical to any of the guide sequences shown in any one in Tables 2A-2B.

In other embodiments, a composition is provided that comprises at least one, e.g., at least two gRNA's comprising guide sequences selected from any two or more of the guide sequences shown in any one in Tables 3A-3B. In some embodiments, the composition comprises at least two gRNA's that each comprise a guide sequence at least 99%, 98%, 97%, 96%, 95%, 94%, 93%, 92%, 91%, or 90% identical to any of the guide sequences shown in any one in Tables 3A-3B.

In some embodiments, the guide RNA compositions of the present disclosure are designed to recognize (e.g., hybridize to) a target sequence. For example, the target sequence may be recognized and cleaved by a provided Cas cleavase comprising a guide RNA. In some embodiments, an RNA-guided DNA binding agent, such as a Cas cleavase, may be directed by a guide RNA to a target sequence, where the guide sequence of the guide RNA hybridizes with the target sequence and the RNA-guided DNA binding agent, such as a Cas cleavase, cleaves the target sequence.

In some embodiments, the selection of the one or more guide RNAs is determined based on target sequences within the target gene. In some embodiments, the compositions comprising one or more guide sequences comprise a guide sequence that is complementary to the corresponding genomic region shown in Tables 2A-2B or 3A-3B, according to coordinates from human reference genome hg38. Guide sequences of further embodiments may be complementary to sequences in the close vicinity of the genomic coordinate listed in any of the Tables 2A-2B or 3A-3B within the target gene. For example, guide sequences of further embodiments may be complementary to sequences that comprise 10 contiguous nucleotides±10 nucleotides of a genomic coordinate listed in Tables 2A-2B or 3A-3B. Without being bound by any particular theory, modifications (e.g., frameshift mutations resulting from indels occurring as a result of a nuclease-mediated DSB) in certain regions of the target gene may be less tolerable than mutations in other regions, thus the location of a DSB is an important factor in the amount or type of protein knockdown that may result. In some embodiments, a gRNA complementary or having complementarity to a target sequence within the target gene used to direct an RNA-guided DNA binding agent to a particular location in the target gene.

In some embodiments, the guide sequence is at least 99%, 98%, 97%, 96%, 95%, 94%, 93%, 92%, 91%, 90%, 85%, or 80% identical to a target sequence present in the target gene. In some embodiments, the guide sequence is at least 99%, 98%, 97%, 96%, 95%, 94%, 93%, 92%, 91%, 90%, 85%, or 80% identical to a target sequence present in the human target gene.

In some embodiments, the target sequence may be complementary to the guide sequence of the guide RNA. In some embodiments, the degree of complementarity or identity between a guide sequence of a guide RNA and its corresponding target sequence may be at least 80%, 85%, 90%, 95%, 96%, 97%, 98%, 99%, or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, or 4 mismatches, where the total length of the guide sequence is 20. In some embodiments, the target sequence and the guide sequence of the gRNA may contain 1-4 mismatches where the guide sequence is 20 nucleotides.

### VI. RNA-Guided DNA Binding Agent

In some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered.

In some embodiments, the composition further comprises an RNA-guided DNA binding agent or a nucleic acid encoding an RNA-guided DNA binding agent.

In some embodiments, the nucleic acid encoding the RNA-guided DNA binding agent comprises an mRNA comprising an open reading frame (ORF) encoding the RNA-guided DNA binding agent.

In some embodiments, the RNA-guided DNA binding agent is a nuclease.

In some embodiments, the RNA-guided DNA binding agent is a Cas9 nuclease.

In some embodiments, the Cas9 is S. pyogenes Cas9.

In some embodiments, the S. pyogenes Cas9 comprises an amino acid sequence having at least 90% identity to a sequence selected from SEQ ID NOs: 853-857 or an ORF encoding a S. pyogenes Cas9 having at least 90% identity to a sequence selected from SEQ ID NOs: 853-857. In some embodiments, the S. pyogenes Cas9 comprises an amino acid sequence having at least 90% identity to SEQ ID NO: 853 or an ORF encoding a S. pyogenes Cas9 having at least 90% identity to SEQ ID NO: 853.

In some embodiments, the ORF encoding the amino acid sequence has at least 85% identity to SEQ ID NOs: 813, 814, 816-819. In some embodiments, the ORF encoding the amino acid sequence has at least 85% identity to SEQ ID NO: 813.

In some embodiments, the Cas9 is Nme Cas9.

In some embodiments, Nme Cas9 comprises an amino acid sequence having at least 90% identity to a sequence selected from SEQ ID NOs: 832-834 or an ORF encoding an Nme Cas9 having at least 90% identity to a sequence selected from SEQ ID NOs: 832-834. In some embodiments, Nme Cas9 comprises an amino acid sequence having at least 90% identity to SEQ ID NO: 832 or an ORF encoding an Nme Cas9 having at least 90% identity to SEQ ID NO: 832.

In some embodiments, the ORF encoding the amino acid sequence has at least 85% identity to a sequence selected from SEQ ID NOs: 802-810. In some embodiments, the ORF encoding the amino acid sequence has at least 85% identity to SEQ ID NO: 802.

In some embodiments, the nuclease has double stranded endonuclease activity.

In some embodiments, the nuclease has nickase activity.

In some embodiments, the nuclease is catalytically inactive.

In some embodiments, the nuclease further comprises a heterologous functional domain.

In some embodiments, the nuclease is a nickase and the heterologous functional domain is a deaminase.

In some embodiments, the deaminase is a cytidine deaminase or an adenine deaminase.

In some embodiments, the deaminase is a cytidine deaminase.

In some embodiments, the deaminase is an apolipoprotein B mRNA editing enzyme (APOBEC) deaminase.

In some embodiments, the nuclease and the deaminase comprise an amino acid sequence having at least 90% identity to a sequence to SEQ ID NO: 831, 835-838, 851, 852, or 858 or an ORF encoding an amino acid sequence having at least 90% identity to SEQ ID NO: 831, 835-838, 851, 852, or 858.

In some embodiments, the ORF encoding the amino acid sequence has at least 85% identity to SEQ ID NOs: 801, 804, 811, 812, or 815.

In some embodiments, the composition described herein further comprises a uracil glycosylase inhibitor (UGI) or nucleic acid encoding a UGI, wherein the nuclease polypeptide does not comprise a UGI or the nucleic acid encoding the polypeptide does not encode a UGI.

In some embodiments, the UGI comprises an amino acid sequence having at least 90% identity to SEQ ID NO: 859 or 860 or an ORF encoding an amino acid sequence having at least 90% identity to SEQ ID NO: 859 or 860.

In some embodiments, the ORF encoding the amino acid sequence has at least 85% identity to a sequence selected from SEQ ID NOs: 823-826, optionally SEQ ID NO: 823.

In some embodiments, the ORF is a modified ORF.

RNA-guided DNA binding agents described herein encompass SpyCas9 and modified and variants thereof.

RNA-guided DNA binding agents described herein encompass Neisseria meningitidis Cas9 (NmeCas9) and modified and variants thereof. In some embodiments, the NmeCas9 is Nme2 Cas9. In some embodiments, the NmeCas9 is Nmel Cas9. In some embodiments, the NmeCas9 is Nme3 Cas9.

Modified versions having one catalytic domain, either RuvC or HNH, that is inactive are termed “nickases.” Nickases cut only one strand on the target DNA, thus creating a single-strand break. A single-strand break may also be known as a “nick.” In some embodiments, the compositions and methods comprise nickases. In some embodiments, the compositions and methods comprise a nickase RNA-guided DNA binding agent, such as a nickase Cas, e.g., a nickase Cas9, that induces a nick rather than a double strand break in the target DNA.

In some embodiments, the NmeCas9 nuclease may be modified to contain only one functional nuclease domain. For example, the RNA-guided DNA binding agent may be modified such that one of the nuclease domains is mutated or fully or partially deleted to reduce its nucleic acid cleavage activity.

In some embodiments, a NmeCas9 nickase having a RuvC domain with reduced activity is used. In some embodiments, a NmeCas9 nickase having an inactive RuvC domain is used. In some embodiments, a NmeCas9 nickase having an HNH domain with reduced activity is used. In some embodiments, a NmeCas9 nickase having an inactive HNH domain is used.

In some embodiments, the nuclease is modified to induce a point mutation or base change, e.g., through deamination.

In some embodiments, the Cas protein comprises a fusion protein comprising a Cas nuclease (e.g., NmeCas9), which is a nickase or is catalytically inactive, linked to a heterologous functional domain. In some embodiments, the Cas protein comprises a fusion protein comprising a catalytically inactive Cas nuclease (e.g., NmeCas9) linked to a heterologous functional domain (see, e.g., WO2014152432). In some embodiments, the catalytically inactive Cas9 is from the N. meningitidis Cas9. In some embodiments, the catalytically inactive Cas comprises mutations that inactivate the Cas.

In some embodiments, the heterologous functional domain is a domain that modifies gene expression, histones, or DNA. In some embodiments, the heterologous functional domain is a transcriptional activation domain or a transcriptional repressor domain. In some embodiments, the nuclease is a catalytically inactive Cas nuclease, such as dCas9.

In some embodiments, the heterologous functional domain is a deaminase, such as a cytidine deaminase or an adenine deaminase. In certain embodiments, the heterologous functional domain is a C to T base converter (cytidine deaminase), such as an apolipoprotein B mRNA editing enzyme (APOBEC) deaminase. A heterologous functional domain such as a deaminase may be part of a fusion protein with a Cas nuclease having nickase activity or a Cas nuclease that is catalytically inactive discussed further below.

The RNA-guided DNA binding agent disclosed herein may further comprise a base-editing domain, such as a deaminase domain, that introduces a specific modification into a target nucleic acid.

In some embodiments, a nucleic acid is provided that comprises an open reading frame encoding a polypeptide comprising a cytidine deaminase (e.g., A3A), a C-terminal NmeCas9 nickase, and a first nuclear localization signal (NLS), wherein the polypeptide does not comprise a uracil glycosylase inhibitor (UGI).

In some embodiments, a second NLS is N-terminal to the NmeCas9 nickase. In some embodiments, the deaminase is N-terminal to an NLS (i.e., the first NLS or the second NLS). In some embodiments, the deaminase is N-terminal to all NLS in the polypeptide. In some embodiments, the deaminase is N-terminal to all NLS in the polypeptide, wherein the polypeptide does not comprise a uracil glycosylase inhibitor (UGI).

In some embodiments, the polynucleotide is DNA or RNA. In some embodiments, the polynucleotide is mRNA. In some embodiments, a polypeptide encoded by the mRNA is provided.

In some embodiments, the polypeptide comprising A3A and an RNA-guided nickase does not comprise a uracil glycosylase inhibitor (UGI).

In some embodiments, a composition is provided comprising a first polypeptide, or an mRNA encoding a first polypeptide, comprising a cytidine deaminase, which is optionally an APOBEC3A deaminase (A3A); a C-terminal NmeCas9 nickase; a first nuclear localization signal (NLS); and, optionally, a second NLS; wherein the first NLS and, when present, the second NLS are located to N-terminal to the sequence encoding the NmeCas9 nickase, wherein the first polypeptide does not comprise a uracil glycosylase inhibitor (UGI); and a second polypeptide, or an mRNA encoding a second polypeptide, comprising a uracil glycosylase inhibitor (UGI), wherein the second polypeptide is different from the first polypeptide.

In some embodiments, methods of modifying a target gene are provided comprising administering the compositions described herein. In some embodiments, the method comprises delivering to a cell a first nucleic acid comprising a first open reading frame encoding a first polypeptide comprising a cytidine deaminase, which is optionally an APOBEC3A deaminase (A3A); a C-terminal NmeCas9 nickase; a first nuclear localization signal (NLS); and, optionally, a second NLS; wherein the first NLS and, when present, the second NLS are located to N-terminal to the sequence encoding the NmeCas9 nickase, wherein the first polypeptide does not comprise a uracil glycosylase inhibitor (UGI), and a second nucleic acid comprising a second open reading frame encoding a uracil glycosylase inhibitor (UGI), wherein the second nucleic acid is different from the first nucleic acid.

In some embodiments, the methods comprise delivering to a cell a polypeptide comprising a deaminase, which is optionally an APOBEC3A deaminase (A3A); a C-terminal NmeCas9 nickase; a first nuclear localization signal (NLS); and a second NLS; wherein the first NLS and the second NLS are located to N-terminal to the sequence encoding the NmeCas9 nickase, wherein the first polypeptide does not comprise a uracil glycosylase inhibitor (UGI), or a nucleic acid encoding the polypeptide, and delivering to the cell a uracil glycosylase inhibitor (UGI), or a nucleic acid encoding the UGI.

In some embodiments, a molar ratio of the mRNA encoding UGI to the mRNA encoding the APOBEC3A deaminase (A3A) and an RNA-guided nickase is from about 1:35 to from about 30:1. In some embodiments, the molar ratio of the mRNA encoding UGI to the mRNA encoding the APOBEC3A deaminase (A3A) and an RNA-guided nickase is not about 1:1.

Similarly, in some embodiments, the molar ratio discussed above for the mRNA encoding the UGI protein to the mRNA encoding the APOBEC3A deaminase (A3A) and an RNA-guided nickase are similar if delivering protein.

In some embodiments, the composition described herein further comprises at least one gRNA. In some embodiments, the composition described herein further comprises two gRNAs. In some embodiments, a composition is provided that comprises an mRNA described herein and at least one gRNA, e.g., two gRNAs. In some embodiments, the gRNA is a single guide RNA (sgRNA). In some embodiments, the gRNA is a dual guide RNA (dgRNA).

In some embodiments, the composition is capable of effecting genome editing upon administration to the subject.

### Cytidine Deaminase; APOBEC3A Deaminase

Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, 2005; Conticello, Genome Biol. 9:229, 2008; Muramatsu et al., J. Biol. Chem. 274:18470-6, 1999); and Carrington et al., Cells 9:1690 (2020)).

In some embodiments, the cytidine deaminase disclosed herein is an enzyme of APOBEC family. In some embodiments, the cytidine deaminase disclosed herein is an enzyme of APOBEC1, APOBEC2, APOBEC4, and APOBEC3 subgroups. In some embodiments, the cytidine deaminase disclosed herein is an enzyme of APOBEC3 subgroup. In some embodiments, the cytidine deaminase disclosed herein is an APOBEC3A deaminase (A3A). In some embodiments, the deaminase comprises an APOBEC3A deaminase.

In some embodiments, an APOBEC3A deaminase (A3A) disclosed herein is a human A3A. In some embodiments, an APOBEC3A deaminase (A3A) disclosed herein is a human A3A. In some embodiments, the A3A is a wild-type A3A.

In some embodiment, the A3A is an A3A variant. A3A variants share homology to wild-type A3A, or a fragment thereof. In some embodiments, a A3A variant has at least about 80% identity, at least about 85% identity, at least about 90% identity, at least about 95% identity, at least about 96% identity, at least about 97% identity, at least about 98% identity, or at least about 99% identity to a wild type A3A. In some embodiments, the A3A variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a wild type A3A. In some embodiments, the A3A variant comprises a fragment of an A3A, such that the fragment has at least about 80% identity, at least about 90% identity, at least about 95% identity, at least about 96% identity, at least about 97% identity, at least about 98% identity, at least about 99% identity, at least about 99.5% identity, or at least about 99.9% identity to the corresponding fragment of a wild-type A3A.

In some embodiments, an A3A variant is a protein having a sequence that differs from a wild-type A3A protein by one or several mutations, such as substitutions, deletions, insertions, one or several single point substitutions. In some embodiments, a shortened A3A sequence could be used, e.g. by deleting N-terminal, C-terminal, or internal amino acids. In some embodiments, a shortened A3A sequence is used where one to four amino acids at the C-terminus of the sequence is deleted. In some embodiments, an APOBEC3A (such as a human APOBEC3A) has a wild-type amino acid position 57 (as numbered in the wild-type sequence). In some embodiments, an APOBEC3A (such as a human APOBEC3A) has an asparagine at amino acid position 57 (as numbered in the wild-type sequence).

In some embodiments, the wild-type A3A is a human A3A (UniPROT accession ID: p31941, SEQ ID NO: 850).

In some embodiments, the A3A disclosed herein comprises an amino acid sequence having at least 80% identity to SEQ ID NO: 850. In some embodiments, the level of identity is at least 85%, at least 87%, at least 90%, at least 95%, at least 98%, at least 99%, or 100%. In some embodiments, the A3A comprises an amino acid sequence having at least 87% identity to SEQ ID NO: 850. In some embodiments, the A3A comprises an amino acid sequence with at least 90% identity to SEQ ID NO: 850. In some embodiments, the A3A comprises an amino acid sequence with at least 95% identity to SEQ ID NO: 850. In some embodiments, the A3A comprises an amino acid sequence with at least 98% identity to SEQ ID NO: 850. In some embodiments, the A3A comprises an amino acid sequence with at least 99% identity to A3A ID NO: 850. In some embodiments, the A3A comprises the amino acid sequence of SEQ ID NO: 850.

In some embodiments, the cytidine deaminase disclosed herein comprises an amino acid sequence having at least 80% identity to SEQ ID NO: 850.

In some embodiments, any of the foregoing levels of identity is at least 90%, at least 95%, at least 98%, at least 99%, or 100%. In some embodiments, the UGI comprises an amino acid sequence with at least 90% identity to SEQ ID NO: 859 or 860. In some embodiments, the UGI comprises an amino acid sequence with at least 95% identity to SEQ ID NO: 859 or 860. In some embodiments, the UGI comprises an amino acid sequence with at least 98% identity to SEQ ID NO: 859 or 860. In some embodiments, the UGI comprises an amino acid sequence with at least 99% identity to SEQ ID NO: 859 or 860. In some embodiments, the UGI comprises the amino acid sequence of SEQ ID NO: 859 or 860.

### Linkers

In some embodiments, the polypeptide comprising the deaminase and the RNA-guided nickase described herein further comprises a linker that connects the deaminase and the RNA-guided nickase. In some embodiments, the linker is a peptide linker. In some embodiments, the nucleic acid encoding the polypeptide comprising the deaminase and the RNA-guided nickase further comprises a sequence encoding the peptide linker. In some embodiments, mRNAs encoding the deaminase-linker-RNA-guided nickase fusion protein are provided.

In some embodiments, the peptide linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids.

In some embodiments, the peptide linker is the 16 residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 901), SGSETPGTSESA (SEQ ID NO: 902), or SGSETPGTSESATPEGGSGGS (SEQ ID NO: 903).

In some embodiments, the peptide linker comprises a (GGGGS) n (SEQ ID NO: 931), a (G) n, an (EAAAK) n (SEQ ID NO: 932), a (GGS) n, an SGSETPGTSESATPES (SEQ ID NO: 901) motif (see, e.g., Guilinger J P, Thompson D B, Liu D R. Fusion of catalytically inactive Cas9 to FokI nuclease improves the specificity of genome modification. Nat. Biotechnol. 2014; 32 (6): 577-82; the entire contents are incorporated herein by reference), or an (XP) n motif, or a combination of any of these, wherein n is independently an integer between 1 and 30. See, WO2015089406, e.g., paragraph [0012], the entire content of which is incorporated herein by reference.

In some embodiments, the peptide linker comprises one or more sequences selected from SEQ ID NOs: 901-991.

VII. Modified gRNAs and mRNAs

In some embodiments, the gRNA is chemically modified. A gRNA comprising one or more modified nucleosides or nucleotides is called a “modified” gRNA or “chemically modified” gRNA, to describe the presence of one or more non-naturally or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) modification or replacement of a naturally occurring nucleobase, including with a non-canonical nucleobase (an exemplary base modification); and (iv) modification of the 3′ end or 5′ end of the oligonucleotide to provide exonuclease stability, e.g., with 2′ O-me, 2′ halide, or 2′ deoxy substituted ribose; or inverted abasic terminal nucleotide, or replacement of phosphodiester with phosphorothioate.

Chemical modifications such as those listed above can be combined to provide modified gRNAs or mRNAs comprising nucleosides and nucleotides (collectively “residues”) that can have two, three, four, or more modifications. For example, a modified residue can have a modified sugar and a modified nucleobase. In certain embodiments, up to 15% of the phosphate groups of a gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, 10%, 15%, preferably at least 20%, 25%, 30%, 35%, 40%, 45%, or 50%) of the positions in a modified gRNA are modified nucleosides or nucleotides. In some embodiments, at least 5% of the positions in the modified guide RNA are modified nucleotides or nucleosides. In some embodiments, at least 10% of the positions in the modified guide RNA are modified nucleotides or nucleosides. In some embodiments at least 15% of the positions in the modified gRNA are modified nucleotides or nucleosides. In some embodiments preferably at least 20% of the positions in the modified gRNA are modified nucleotides or nucleosides. In some embodiments, no more than 65% of the positions in the modified gRNA are modified nucleotides. In some embodiments, no more than 55% of the positions in the modified gRNA are modified nucleotides. In some embodiments, no more than 50% of the positions in the modified gRNA are modified nucleotides. In some embodiments, 10-70% of the positions in the modified gRNA are modified nucleotides. In some embodiments, 20-70% of the positions in the modified gRNA are modified nucleotides. In some embodiments, 20-50% of the positions in the modified gRNA are modified nucleotides and the nuclease is a Spy Cas9 nuclease. In some embodiments, 30-70% of the positions in the modified gRNA are modified nucleotides and the nuclease is an Nme Cas9 nuclease.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, borano phosphate esters, methyl phosphonates, phosphoroamidates, phosphodithioate, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications, e.g., an amide linkage. In some embodiments, the charged phosphate group can be replaced by a neutral moiety. Examples of moieties which can replace the phosphate group can include, without limitation, e.g., methyl phosphonate, carboxymethyl, carbamate, amide, thioether. Further examples of moieties which can replace the phosphate group can include, without limitation, e.g., ethylene oxide linker, sulfonate, sulfonamide, thioformacetal, formacetal, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂) n-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C₃′ bond. In some embodiments, the 2′ hydroxyl group modification can include the methoxyethyl group (MOE), (OCH₂CH₂OCH₃, e.g., a PEG derivative). 2′ modifications can include hydrogen (i.e. deoxyribose sugars); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides. As used herein, a single abasic sugar is not understood to result in a discontinuity of a duplex.

In certain embodiments, 2′ modifications, include, for example, modifications include 2′-OMe, 2′-F, 2′-H, optionally 2′-O-Me.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, and pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA or tracr RNA. In embodiments comprising an sgRNA, one or more residues at one or both ends of the sgRNA may be chemically modified, or internal nucleosides may be modified, or the sgRNA may be chemically modified throughout. Certain embodiments comprise a 5′ end modification. Certain embodiments comprise a 3′ end modification. Certain embodiments comprise a 5′ end modification and a 3′ end modification.

In some embodiments, the guide RNAs disclosed herein comprise one of the structures/modification patterns disclosed in US20170114334, the contents of which are hereby incorporated by reference in their entirety. In some embodiments, the guide RNAs disclosed herein comprise one of the structures/modification patterns disclosed in WO2017/136794, the contents of which are hereby incorporated by reference in their entirety. In some embodiments, the guide RNAs disclosed herein comprise one of the modification patterns disclosed in WO2018/107028, the contents of which are hereby incorporated by reference in their entirety. In some embodiments, the guide RNAs disclosed herein comprise one of the structures/modification patterns disclosed in WO2019/237069, the contents of which are hereby incorporated by reference in their entirety. In some embodiments, the guide RNAs disclosed herein comprise one of the structures/modification patterns disclosed in WO2021/119275, the contents of which are hereby incorporated by reference in their entirety. In some embodiments, the guide RNAs disclosed herein comprise one of the structures/modification patterns disclosed in WO2023081687A1, the contents of which are hereby incorporated by reference in their entirety. In some embodiments, the guide RNAs disclosed herein comprise one of the structures/modification patterns disclosed in WO2022261292, the contents of which are hereby incorporated by reference in their entirety.

The terms “mA,” “mC,” “mU,” or “mG” may be used to denote a nucleotide that has been modified with 2′-O-Me. The terms “fA,” “fC,” “fU,” or “fG” may be used to denote a nucleotide that has been substituted with 2′-F. A “*” may be used to depict a PS modification.

The terms A*, C*, U*, or G* may be used to denote a nucleotide that is linked to the next (e.g., 3′) nucleotide with a PS bond.

The terms “mA*,” “mC*,” “mU*,” or “mG*” may be used to denote a nucleotide that has been substituted with 2′-O-Me and that is linked to the next (e.g., 3′) nucleotide with a PS bond.

Any of the modifications described below may be present in the gRNAs and mRNAs described herein.

In the context of chemically modified sequences, “A,” “C,” “G,” “N,” and “U” denote an RNA nucleotide, i.e., 2′-OH with a phosphodiesterase linkage to the 3′ nucleotide.

The terms “mA,” “mC,” “mU,” or “mG” are used to denote an adenine, cytosine, uridine, or guanidine nucleotide, respectively, that has been modified with 2′-O-Me.

Modification with 2′-O-methyl can be depicted as follows:

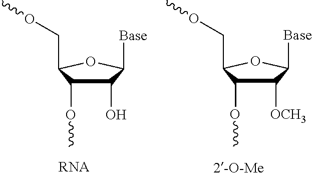

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fU,” or “fG” are used to denote a nucleotide that has been substituted with 2′-F.

Substitution of 2′-F can be depicted as follows:

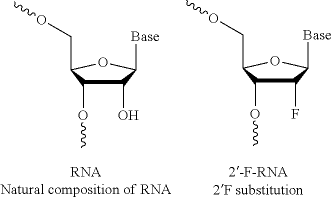

Phosphorothioate (PS) linkage or bond refers to a bond where a sulfur is substituted for one non-bridging phosphate oxygen in a phosphodiester linkage, for example in the bonds between nucleotides bases. When phosphorothioates are used to generate oligonucleotides, the modified oligonucleotides may also be referred to as S-oligos.

A “*” is used to denote a PS modification. In this application, the terms A*, C*, U*, or G* may be used to denote a nucleotide that is linked to the next (e.g., 3′) nucleotide with a PS bond.

In this application, the terms “mA*,” “mC*,” “mU*,” or “mG*” are used to denote a nucleotide that has been substituted with 2′-O-Me and that is linked to the next (e.g., 3′) nucleotide with a PS bond.

The diagram below shows the substitution of S-into a non-bridging phosphate oxygen, generating a PS bond in lieu of a phosphodiester bond:

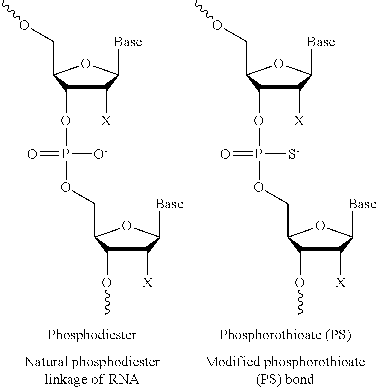

Abasic nucleotides refer to those which lack nitrogenous bases. The diagram below depicts an oligonucleotide with an abasic (also known as apurinic) site that lacks a base. As used herein, the presence of a single abasic site is not considered to disrupt a duplex, e.g., a duplex formed between the guide sequence of a guide RNA and a target site in the genome:

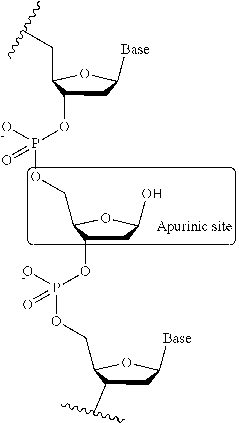

Inverted bases refer to those with linkages that are inverted from the normal 5′ to 3′ linkage (i.e., either a 5′ to 5′ linkage or a 3′ to 3′ linkage). Such inverted bases can only be present as a terminal nucleotide. In chemical synthesis methods performed 3′ to 5′, inverted bases do not have 5′ hydroxy available to grow the chain. For example:

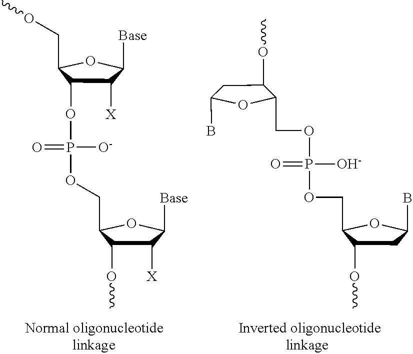

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl(2′-O-Me) modified nucleotide. In some embodiments, the first three nucleotides at the 5′ terminus, and the last three nucleotides at the 3′ terminus comprise a 2′-fluoro (2′-F) modified nucleotide. In some embodiments, the first three nucleotides at the 5′ terminus, and the last three nucleotides at the 3′ terminus comprise an inverted abasic nucleotide.

In some embodiments, the Spy guide RNA comprises a modified sgRNA. In some embodiments, the sgRNA comprises the modification pattern shown in Tables 5A-5B, for example
mN*mN*mN*NNNNNNNNNNNNNNNNNGUUUUAGAmGmCmUmAmGmAmAmAmU mAmGmCAAGUUAAAAUAAGGCUAGUCCGUUAUCACGAAAGGGCACCGAGUCG GmU*mG*mC*mU (SEQ ID NO: 669); or
mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 658) where each A, C, G, U, and N is an RNA nucleotide, 2′-OH and phosphodiester linkage to the 3′ nucleotide, m indicates a 2′-O-methyl(2′-O-Me) modified nucleotide, and * indicates a phosphorothioate linkage between nucleotides, and where the totality of the N's comprise a guide sequence that directs a nuclease to a target sequence, e.g., the target sequence that is complementary to a guide sequence. In certain embodiments, the guide sequence comprises a guide sequence described in Tables 3A-3B. Also encompassed herein are guide RNAs combining any of the guide sequences of Tables 3A-3B (combined with a conserved portion of an sgRNA, e.g., a sequence of Tables 4A and 5A.

In some embodiments, the Nme guide RNA comprises a modified sgRNA. In some embodiments, the sgRNA comprises the modification pattern shown in Table 7A-7B, for example,
mN*mN*mN*mNmNNNmNmNNmNNmNNNNNmNNNNmNNNmGUUGmUmAmGmC UCCCmUmGmAmAmAmCmCGUUmGmCUAmCAAU*AAGmGmCCmGmUmCmGmA mAmAmGmAmUGUGCmCGmCAAmCGCUCUmGmCCmUmUmCmUGGCAUCG*mU* mU (SEQ ID NO: 731); or
mN*mN*mN*mNmNNNmNmNNmNNmNNNNNmNNNNmNNNmGUUGmUmAmGmC UCCCmUmGmAmAmAmCmCGUUmGmCUAmCAAUAAGmGmCCmGmUmCmGmAm AmAmGmAmUGUGCmCGmCAAmCGCUCUmGmCCmUmUmCmUGGCAUCG*mU*m U (SEQ ID NO: 732), where each A, C, G, U, and Nis an RNA nucleotide, 2′-OH and phosphodiester linkage to the 3′ nucleotide, m indicates a 2′-O-methyl(2′-O-Me) modified nucleotide, and * indicates a phosphorothioate linkage between nucleotides, and where the totality of the N's comprise a guide sequence that directs a nuclease to a target sequence in a target gene. In certain embodiments, the guide sequence comprises a guide sequence shown in Tables 2A-2B. Also encompassed herein are guide RNAs combining any of the guide sequences of Tables 2A-2B (combined with a conserved portion of an sgRNA, e.g., a sequence of Tables 6A and 7A.

As noted above, in some embodiments, a composition or formulation disclosed herein comprises an mRNA comprising an open reading frame (ORF) encoding an RNA-guided DNA binding agent, such as a Cas nuclease, e.g. Cas9 nuclease, as described herein. In some embodiments, an mRNA comprising an ORF encoding an RNA-guided DNA binding agent, such as a Cas nuclease, e.g. Cas9 nuclease, is provided, used, or administered. In some embodiments, the ORF encoding an RNA-guided DNA nuclease is a “modified RNA-guided DNA binding agent ORF” or simply a “modified ORF,” which is used as shorthand to indicate that the ORF is modified.

In some embodiments, the mRNA or modified ORF may comprise a modified uridine at least at one, a plurality of, or all uridine positions. In some embodiments, the modified uridine is a uridine modified at the 5 position, e.g., with a halogen, methyl, or ethyl. In some embodiments, the modified uridine is a pseudouridine modified at the 1 position, e.g., with a halogen, methyl, or ethyl. The modified uridine can be, for example, pseudouridine, N1-methyl-pseudouridine, 5-methoxyuridine, 5-iodouridine, or a combination thereof. In some embodiments, the modified uridine is 5-methoxyuridine. In some embodiments, the modified uridine is 5-iodouridine. In some embodiments, the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, an mRNA disclosed herein comprises a 5′ cap, such as a Cap0, Cap1, or Cap2. A 5′ cap is generally a 7-methylguanine ribonucleotide (which may be further modified, as discussed below e.g. with respect to ARCA) linked through a 5′-triphosphate to the 5′ position of the first nucleotide of the 5′-to-3′ chain of the mRNA, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111 (33): 12025-30; Abbas et al. (2017) Proc Natl Acad Sci USA 114 (11): E2106-E2115. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl (3′-O-methyl) GpppG and 7-methyl (3′deoxy) GpppG,” RNA 7:1486-1495. The ARCA structure is shown below.

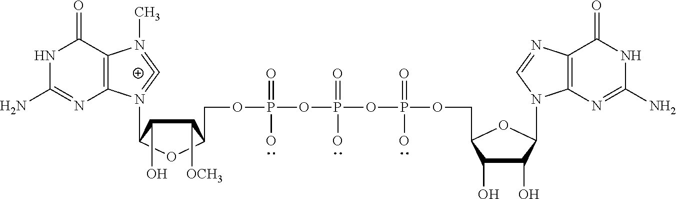

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively, or CleanCap AU: TriLink Biotechnologies as Cat. Nos. N-7114. The CleanCap™ AG structure is shown below.

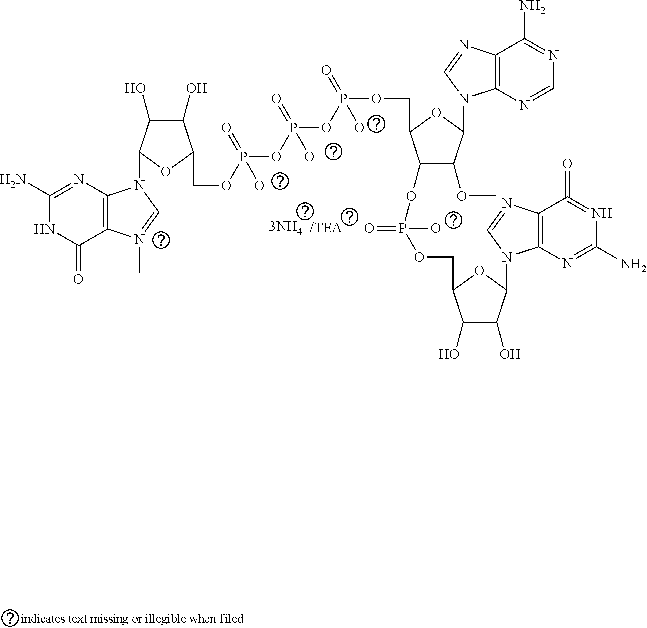

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479.

In some embodiments, the mRNA further comprises a poly-adenylated (poly-A) tail. In some embodiments, the poly-A tail comprises at least 20, 30, 40, 50, 60, 70, 80, 90, or 100 adenines, optionally up to 300 adenines. In some embodiments, the poly-A tail comprises 95, 96, 97, 98, 99, or 100 adenine nucleotides. In some embodiments, the poly-A tail includes non-adenine nucleotides, i.e., is an interrupted poly-A tail. In certain embodiments, the poly-A tail is interrupted by a non-adenine nucleotide about every 40, 50, 60, 70, 80, or 90 nucleotides. In certain embodiments, the poly-A tail is interrupted by a non-adenine nucleotide about every 50 nucleotides.

### VIII. Ribonucleoprotein Complex

In some embodiments, a composition is encompassed comprising one or more sgRNAs comprising one or more guide sequences from Table 2A or 3A or one or more sgRNAs from Table 2B or 3B and an RNA-guided DNA binding agent, e.g., a nuclease, such as a Cas nuclease, such as Cas9. In some embodiments, the RNA-guided DNA-binding agent has cleavase activity, which can also be referred to as double-strand endonuclease activity. In some embodiments, the RNA-guided DNA-binding agent comprises a Cas nuclease. Examples of Cas9 nucleases include those of the type II CRISPR systems of S. pyogenes, Neisseria meningitidis, and other prokaryotes as known in the art, and modified (e.g., engineered or mutant) versions thereof.

In some embodiments, the Cas nuclease is the Cas9 nuclease from Streptococcus pyogenes. In some embodiments, the Cas nuclease is the Cas9 nuclease from Neisseria meningitidis.

In some embodiments, the gRNA together with an RNA-guided DNA binding agent is called a ribonucleoprotein complex (RNP). In some embodiments, the RNA-guided DNA binding agent is a Cas nuclease. In some embodiments, the gRNA together with a Cas nuclease is called a Cas RNP. In some embodiments, the RNP comprises Type-I, Type-II, or Type-III components. In some embodiments, the Cas nuclease is the Cas9 protein from the Type-II CRISPR/Cas system. In some embodiment, the gRNA together with Cas9 is called a Cas9 RNP.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain or more than one HNH domain. In some embodiments, the Cas9 protein is a wild type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22: 163 (3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015).

In some embodiments, the polypeptide comprises, from N to C terminus, an optional NLS, a cytidine deaminase (e.g., APOBEC3A), an optional linker, and an Nme Cas9 nickase with an amino acid substitution in the HNH or HNH-like nuclease domain, e.g., a D16A NmeCas9 nickase. In some embodiments, the polypeptide comprises, from N to C terminus, an optional NLS, a cytidine deaminase (e.g., APOBEC3A), an optional linker, and an Nme Cas9 nickase with an amino acid substitution in the HNH or HNH-like nuclease domain, e.g., a D16A Nme2Cas9 nickase. In some embodiments, the polypeptide comprises, from N to C terminus, first and second NLSs, a cytidine deaminase (e.g., APOBEC3A), an optional linker, and an Nme Cas9 nickase with an amino acid substitution in the HNH or HNH-like nuclease domain, e.g., a D16A NmeCas9 nickase. In some embodiments, the polypeptide comprises, from N to C terminus, first and second NLSs, a cytidine deaminase (e.g., APOBEC3A), an optional linker, and an Nme Cas9 nickase with an amino acid substitution in the HNH or HNH-like nuclease domain, e.g., a D16A Nme2Cas9 nickase. In some embodiments, the polypeptide comprises, from N to C terminus, A first NLS, a cytidine deaminase (e.g., APOBEC3A), a second NLS, an optional linker, and an Nme Cas9 nickase with an amino acid substitution in the HNH or HNH-like nuclease domain, e.g., a D16A NmeCas9 nickase. In some embodiments, the polypeptide comprises, from N to C terminus, A first NLS, a cytidine deaminase (e.g., APOBEC3A), a second NLS, an optional linker, and an Nme Cas9 nickase with an amino acid substitution in the HNH or HNH-like nuclease domain, e.g., a D16A Nme2Cas9 nickase.

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US20140186958; US20150166980; and US20190338308.

In some embodiments, the RNA-guided DNA-binding agent comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-5 NLS(s). In some embodiments, the RNA-guided DNA-binding agent may be fused with 2, 3, or 4 NLS(s). In some embodiments, the RNA-guided DNA-binding agent may be fused with two NLS(s). In some embodiments, the RNA-guided DNA-binding agent may be fused with one NLS. Where one NLS is used, the NLS may be linked at the N-terminus or the C-terminus of the RNA-guided DNA-binding agent sequence. In some embodiments, the NLS is not linked to the C-terminus. It may also be inserted within the RNA-guided DNA binding agent sequence. In certain circumstances, at least the two NLSs are the same (e.g., two SV40 NLSs). In certain embodiments, at least two different NLSs are present the RNA-guided DNA binding agent. In some embodiments, the RNA-guided DNA-binding agent is fused to two SV40 NLS sequences linked at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS.

In some embodiments, the NLS may be SV40 NLS. Exemplary SV40 NLS sequence may be SV40 NLS, PKKKRKV (SEQ ID NO: 916) or PKKKRRV (SEQ ID NO: 928). In some embodiments, the NLS may be a bipartite sequence, such as the NLS of nucleoplasmin, KRPAATKKAGQAKKKK (SEQ ID NO: 929). In some embodiments, the NLS sequence may comprise LAAKRSRTT (SEQ ID NO: 917), QAAKRSRTT (SEQ ID NO: 918), PAPAKRERTT (SEQ ID NO: 919), QAAKRPRTT (SEQ ID NO: 920), RAAKRPRTT (SEQ ID NO: 921), AAAKRSWSMAA (SEQ ID NO: 922), AAAKRVWSMAF (SEQ ID NO: 923), AAAKRSWSMAF (SEQ ID NO: 924), AAAKRKYFAA (SEQ ID NO: 925), RAAKRKAFAA (SEQ ID NO: 926), or RAAKRKYFAV (SEQ ID NO: 927). The NLS may be a snurportin-1 importin-β (IBB domain, e.g. an SPN1-impβ sequence. See Huber et al., 2002, J. Cell Bio., 156, 467-479. In a specific embodiment, a single PKKKRKV (SEQ ID NO: 916). In some embodiments, the first and second NLS are independently selected from an SV40 NLS, a nucleoplasmin NLS, a bipartite NLS, a c-myc like NLS, and an NLS comprising the sequence KTRAD (SEQ ID NO: 1005). In certain embodiments, the first and second NLSs may be the same (e.g., two SV40 NLSs). In certain embodiments, the first and second NLSs may be different.

In some embodiments, the first NLS is a SV40NLS and the second NLS is a nucleoplasmin NLS.

In some embodiments, the SV40 NLS comprises a sequence of PKKKRKVE (SEQ ID NO: 996) or KKKRKVE (SEQ ID NO: 997). In some embodiments, the nucleoplasmin NLS comprises a sequence of KRPAATKKAGQAKKKK (SEQ ID NO: 929). In some embodiments, the bipartite NLS comprises a sequence of KRTADGSEFESPKKKRKVE (SEQ ID NO: 998). In some embodiments, the c-myc like NLS comprises a sequence of PAAKKKKLD (SEQ ID NO: 999).

One or more linkers are optionally included at the fusion site of the NLS to the nuclease, or between NLS when more than one is present.

In some embodiments, one or more NLS(s) according to any of the foregoing embodiments are present in the RNA-guided DNA-binding agent in combination with one or more additional heterologous functional domains. One or more linkers are optionally included at the fusion site.

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub1 in S. cerevisiae), human leukocyte antigen F-associated (FAT10), autophagy-8 (ATG8) and-12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, Zs Yellow1), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag or an epitope tag. Non-limiting exemplary tags include glutathione-S-transferase (GST), chitin binding protein (CBP), maltose binding protein (MBP), thioredoxin (TRX), poly (NANP), tandem affinity purification (TAP) tag, myc, AcV5, AU1, AU5, E, ECS, E², FLAG, HA, nus, Softag 1, Softag 3, Strep, SBP, Glu-Glu, HSV, KT3, S, S1, T7, V5, VSV-G, 6xHis, 8xHis, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In further embodiments, the heterologous functional domain may be an effector domain. When the RNA-guided DNA-binding agent is directed to its target sequence, e.g., when a Cas nuclease is directed to a target sequence by a gRNA, the effector domain may modify or affect the target sequence. In some embodiments, the effector domain may be chosen from a nucleic acid binding domain, a nuclease domain (e.g., a non-Cas nuclease domain), an epigenetic modification domain, a transcriptional activation domain, and a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA.

In some embodiments, the heterologous functional domain is a deaminase, such as a cytidine deaminase or an adenine deaminase. In certain embodiments, the heterologous functional domain is a C to T base converter (cytidine deaminase), such as an apolipoprotein B mRNA editing enzyme (APOBEC) deaminase.

In some embodiments, the heterologous functional domain comprises a APOBEC3 deaminase. In some embodiments, the APOBEC3 deaminase is APOBEC3A (A3A). In some embodiments, the A3A is a human A3A. In some embodiments, the A3A is a wild-type A3A.

### IX. Determination of Efficacy of Guide RNAs

In some embodiments, the efficacy of a guide RNA is determined when delivered or expressed together with other components (e.g., an RNA-guided DNA binding agent) forming an RNP. In some embodiments, the guide RNA is expressed together with an RNA-guided DNA binding agent, such as a Cas protein, e.g., Cas9. In some embodiments, the guide RNA is delivered to or expressed in a cell line that already stably expresses an RNA-guided DNA nuclease, such as a Cas nuclease or nickase, e.g., Cas9 nuclease or nickase. In some embodiments the guide RNA is delivered to a cell as part of a RNP. In some embodiments, the guide RNA is delivered to a cell along with a mRNA encoding an RNA-guided DNA nuclease, such as a Cas nuclease or nickase, e.g., Cas9 nuclease or nickase.

As described herein, use of an RNA-guided DNA nuclease and a guide RNA disclosed herein can lead to DSBs, SSBs, or site-specific binding that results in nucleic acid modification in the DNA or pre-mRNA which can produce errors in the form of insertion/deletion (indel) mutations upon repair by cellular machinery. Many mutations due to indels alter the reading frame, introduce premature stop codons, or induce exon skipping and, therefore, produce a non-functional protein.

In some embodiments, the efficacy of particular guide RNAs is determined based on in vitro models. In some embodiments, the in vitro model is T cell line. In some embodiments, the in vitro model is HEK293 T cells. In some embodiments, the in vitro model is HEK293 cells stably expressing Cas9 (HEK293_Cas9). In some embodiments, the in vitro model is a lymphoblastoid cell line. In some embodiments, the in vitro model is primary human T cells. In some embodiments, the in vitro model is primary human B cells. In some embodiments, the in vitro model is primary human peripheral blood lymphocytes. In some embodiments, the in vitro model is primary human peripheral blood mononuclear cells.

In some embodiments, the number of off-target sites at which a deletion or insertion occurs in an in vitro model is determined, e.g., by analyzing genomic DNA from the cells transfected in vitro with Cas9 mRNA and the guide RNA. In some embodiments, such a determination comprises analyzing genomic DNA from cells transfected in vitro with Cas9 mRNA, the guide RNA, and a donor oligonucleotide. Exemplary procedures for such determinations are provided in the working examples below.

In some embodiments, the efficacy of particular gRNAs is determined across multiple in vitro cell models for a guide RNA selection process. In some embodiments, a cell line comparison of data with selected guide RNAs is performed. In some embodiments, cross screening in multiple cell models is performed.

In some embodiments, the efficacy of a guide RNA is evaluated by on target cleavage efficiency. In some embodiments, the efficacy of a guide RNA is measured by percent editing at the target location. In some embodiments, deep sequencing may be utilized to identify the presence of modifications (e.g., insertions, deletions) introduced by gene editing. Indel percentage can be calculated from next generation sequencing “NGS.”

In some embodiments, the efficacy of a guide RNA is measured by the number or frequency of indels at off-target sequences within the genome of the target cell type. In some embodiments, efficacious guide RNAs are provided which produce indels at off target sites at very low frequencies (e.g., <5%) in a cell population or relative to the frequency of indel creation at the target site. Thus, the disclosure provides for guide RNAs which do not exhibit off-target indel formation in the target cell type (e.g., T cells or B cells), or which produce a frequency of off-target indel formation of <5% in a cell population or relative to the frequency of indel creation at the target site. In some embodiments, the disclosure provides guide RNAs which do not exhibit any off target indel formation in the target cell type (e.g., T cells or B cells). In some embodiments, guide RNAs are provided which produce indels at less than 5 off-target sites, e.g., as evaluated by one or more methods described herein. In some embodiments, guide RNAs are provided which produce indels at less than or equal to 4, 3, 2, or 1 off-target site(s) e.g., as evaluated by one or more methods described herein. In some embodiments, the off-target site(s) does not occur in a protein coding region in the target cell (e.g., T cells or B cells) genome.

In some embodiments, linear amplification is used to detect gene editing events, such as the formation of insertion/deletion (“indel”) mutations, translocations, and homology directed repair (HDR) events in target DNA. For example, linear amplification with a unique sequence-tagged primer and isolating the tagged amplification products (herein after referred to as “UnIT,” or “Unique Identifier Tagmentation” method) may be used.

In some embodiments, the efficacy of a guide RNA is measured by the number of chromosomal rearrangements within the target cell type. Kromatid dGH assay may used to detect chromosomal rearrangements, including e.g., translocations, reciprocal translocations, translocations to off-target chromosomes, deletions (i.e., chromosomal rearrangements where fragments were lost during the cell replication cycle due to the editing event). In some embodiments, the target cell type has less than 10, less than 8, less than 5, less than 4, less than 3, less than 2, or less than 1 chromosomal rearrangement. In some embodiments, the target cell type has no chromosomal rearrangements.

### X. Delivery of Compositions

Lipid nanoparticles (LNPs) are a well-known means for delivery of nucleotide and protein cargo and may be used for delivery of the guide RNAs, compositions, or pharmaceutical formulations disclosed herein. In some embodiments, the LNP compositions deliver nucleic acid, protein, or nucleic acid together with protein.

In some embodiments, the present disclosure provides a method for delivering any one of the gRNAs disclosed herein to a cell, wherein the gRNA is formulated as an LNP. In some embodiments, the LNP comprises the gRNA and a Cas9 or an mRNA encoding Cas9.

In some embodiments, the present disclosure provides a composition comprising any one of the gRNAs disclosed and an LNP. In some embodiments, the composition further comprises a Cas9 or an mRNA encoding Cas9.

In some embodiments, the LNP compositions comprise cationic lipids. In some embodiments, the LNP compositions comprise (9Z,12Z)-3-((4,4-bis(octyloxy) butanoyl)oxy)-2-((((3-(diethylamino) propoxy) carbonyl)oxy) methyl) propyl octadeca-9,12-dienoate, also called 3-((4,4-bis(octyloxy) butanoyl)oxy)-2-((((3-(diethylamino) propoxy) carbonyl)oxy) methyl) propyl (9Z,12Z)-octadeca-9,12-dienoate) or another ionizable lipid. See, e.g., lipids of WO/2017/173054 and references described therein. In some embodiments, the LNP compositions comprise molar ratios of a cationic lipid amine to RNA phosphate (N: P) of about 4.5, 5.0, 5.5, 6.0, or 6.5. In some embodiments, the term cationic and ionizable in the context of LNP lipids is interchangeable, e.g., wherein ionizable lipids are cationic depending on the pH.

In some embodiments, the LNP comprises a lipid component, and the lipid component comprises: about 35 mol % Lipid A; about 15 mol % neutral lipid (e.g., distearoylphosphatidylcholine (DSPC)); about 47.5 mol % helper lipid (e.g., cholesterol); and about 2.5 mol % stealth lipid (e.g., 1,2-dimyristoyl-rac-glycero-3-methylpolyoxyethylene glycol 2000 (PEG2k-DMG)), and wherein the N/P ratio of the LNP composition is about 3-7.

In some embodiments, the LNP comprises a lipid component, and the lipid component comprises ionizable lipid ((9Z,12Z)-3-((4,4-bis(octyloxy) butanoyl)oxy)-2-((((3-(diethylamino) propoxy) carbonyl)oxy) methyl) propyl octadeca-9,12-dienoate, also called 3-((4,4-bis(octyloxy) butanoyl)oxy)-2-((((3-(diethylamino) propoxy) carbonyl)oxy) methyl) propyl (9Z,12Z)-octadeca-9,12-dienoate), cholesterol, DSPC, and PEG2k-DMG in a molar ratio of 50% ionizable lipid, 38% cholesterol, 9% DSPC, and 3% PEG2k-DMG.

In some embodiments, the gRNAs disclosed herein are formulated as LNP compositions for use in preparing a medicament for treating a disease or disorder.

Electroporation is a well-known means for delivery of cargo, and any electroporation methodology may be used for delivery of any one of the gRNAs disclosed herein. In some embodiments, electroporation may be used to deliver any one of the gRNAs disclosed herein and Cas9 or an mRNA encoding Cas9.

In some embodiments, the present disclosure comprises a method for delivering any one of the gRNAs disclosed herein to an ex vivo cell, wherein the gRNA is formulated as an LNP or not formulated as an LNP. In some embodiments, the LNP comprises the gRNA and a Cas9 or an mRNA encoding Cas9.

In some embodiments, the guide RNA compositions described herein, alone or encoded on one or more vectors, are formulated in or administered via a lipid nanoparticle; see e.g., WO/2017/173054 and WO 2019/067992, the contents of which are hereby incorporated by reference in their entirety.

In certain embodiments, the present disclosure comprises DNA or RNA vectors encoding any of the guide RNAs comprising any one or more of the guide sequences described herein. In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and nucleic acids encoding an RNA-guided DNA nuclease, which can be a nuclease such as Cas9. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA nuclease, which can be a Cas nuclease, such as Cas9. In some embodiments, the vector comprises one or more nucleotide sequence(s) encoding a crRNA, a trRNA, and an mRNA encoding an RNA-guided DNA nuclease, which can be a Cas protein, such as, Cas9. In one embodiment, the Cas9 is from S. pyogenes (i.e., Spy Cas9).

In one embodiment, the Cas9 nuclease is from N. meningitidis (i.e., Nme Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the components can be introduced into a cell as naked nucleic acid, as nucleic acid complexed with an agent such as a liposome or poloxamer, or they can be delivered by viral vectors (e.g., adenovirus, AAV, herpesvirus, retrovirus, lentivirus). Methods and compositions for non-viral delivery of nucleic acids include electroporation, lipofection, microinjection, biolistics, virosomes, liposomes, immunoliposomes, LNPs, polycation or lipid: nucleic acid conjugates, naked nucleic acid (e.g., naked DNA/RNA), artificial virions, and agent-enhanced uptake of DNA. Sonoporation using, e.g., the Sonitron 2000 system (Rich-Mar) can also be used for delivery of nucleic acids.

### XI. Exemplary Cell Types

In some embodiments, methods and compositions disclosed herein genetically modify a cell. In some embodiments, the cell is an allogeneic cell. In some embodiments the cell is a human cell. In some embodiments the genetically modified cell is referred to as an engineered cell. An engineered cell refers to a cell (or progeny of a cell) comprising an engineered genetic modification, e.g. that has been contacted with a genomic editing system and genetically modified by the genomic editing system. The terms “engineered cell” and “genetically modified cell” are used interchangeably throughout. The engineered cell may be any of the exemplary cell types disclosed herein.

In some embodiments, the engineered cell is an immune cell. As used herein, “immune cell” refers to a cell of the immune system, including e.g., a lymphocyte (e.g., T-cell, B cell, natural killer cell (“NK cell”, and NKT cell, or iNKT cell)), monocyte, macrophage, mast cell, dendritic cell, or granulocyte (e.g., neutrophil, eosinophil, and basophil). In some embodiments, the cell is a primary immune cell. In some embodiments, the immune system cell may be selected from CD3⁺, CD4⁺ and CD8⁺ T-cells, regulatory T-cells (Tregs), B cells, NK cells, and dendritic cells (DC). In some embodiments, the immune cell is allogeneic.

In some embodiments, the cell is a lymphocyte. In some embodiments, the cell is an adaptive immune cell. In some embodiments, the cell is a T-cell. In some embodiments, the cell is a B cell. In some embodiments, the cell is a NK cell. In some embodiments, the lymphocyte is allogeneic.

As used herein, a T-cell can be defined as a cell that expresses a T-cell receptor (“TCR” or “αβ TCR” or “γδ TCR”), however in some embodiments, the TCR of a T-cell may be genetically modified to reduce its expression (e.g., by genetic modification to the TRAC gene), therefore expression of the protein CD3 may be used as a marker to identify a T-cell by standard flow cytometry methods. CD3 is a multi-subunit signaling complex that associates with the TCR. Thus, a T-cell may be referred to as CD3+. In some embodiments, a T-cell is a cell that expresses a CD3+ marker and either a CD4+ or CD8+ marker. In some embodiments, the T-cell is allogeneic.

In some embodiments, the T-cell expresses the glycoprotein CD8 and therefore is CD8+ by standard flow cytometry methods and may be referred to as a “cytotoxic” T-cell. In some embodiments, the T-cell expresses the glycoprotein CD4 and therefore is CD4+ by standard flow cytometry methods and may be referred to as a “helper” T-cell. CD4+ T-cells can differentiate into subsets and may be referred to as a Th1 cell, Th2 cell, Th9 cell, Th17 cell, Th22 cell, T regulatory (“Treg”) cell, or T follicular helper cells (“Tfh”). Each CD4+ subset releases specific cytokines that can have either proinflammatory or anti-inflammatory functions, survival or protective functions. A T-cell may be isolated from a subject by CD4+ or CD8+ selection methods.

In some embodiments, the T-cell is a memory T-cell. In the body, a memory T-cell has encountered antigen. A memory T-cell can be located in the secondary lymphoid organs (central memory T-cells) or in recently infected tissue (effector memory T-cells). A memory T-cell may be a CD8+ T-cell. A memory T-cell may be a CD4+ T-cell.

As used herein, a “central memory T-cell” can be defined as an antigen-experienced T-cell, and for example, may expresses CD62L and CD45RO. A central memory T-cell may be detected as CD62L+ and CD45RO+. Central memory T-cells may also express CCR7, therefore may be detected as CCR7+ by standard flow cytometry methods.

As used herein, an “early stem-cell memory T-cell” (or “stem cell-like memory T cells”, or “Tscm”) can be defined as a T-cell that expresses CD27 and CD45RA, and therefore is CD27+ and CD45RA+ by standard flow cytometry methods. A Tscm does not express the CD45 isoform CD45RO, therefore a Tscm will further be CD45RO- if stained for this isoform by standard flow cytometry methods. A CD45RO-CD27+ cell is therefore also an early stem-cell memory T-cell. Tscm cells further express CD62L and CCR7, therefore may be detected as CD62L+ and CCR7+ by standard flow cytometry methods. Early stem-cell memory T-cells have been shown to correlate with increased persistence and therapeutic efficacy of cell therapy products.

In some embodiments, the cell is a B cell. As used herein, a “B cell” can be defined as a cell that expresses CD19 or CD20, or B cell mature antigen (“BCMA”), and therefore a B cell is CD19+, or CD20+, or BCMA+ by standard flow cytometry methods. A B cell is further negative for CD3 and CD56 by standard flow cytometry methods. The B cell may be a plasma cell. The B cell may be a memory B cell. The B cell may be a naïve B cell. The B cell may be IgM+, or has a class-switched B cell receptor (e.g., IgG+, or IgA+). In some embodiments, the B cell is allogeneic.

In some embodiments, the cell is a mononuclear cell, such as from bone marrow or peripheral blood. In some embodiments, the cell is a peripheral blood mononuclear cell (“PBMC”). In some embodiments, the cell is a PBMC, e.g. a lymphocyte or monocyte. In some embodiments, the cell is a peripheral blood lymphocyte (“PBL”). In some embodiments, the mononuclear cell is allogeneic.

Cells used in ACT or tissue regenerative therapy are included, such as stem cells, progenitor cells, and primary cells. Stem cells, for example, include pluripotent stem cells (PSCs); induced pluripotent stem cells (iPSCs); embryonic stem cells (ESCs); mesenchymal stem cells (MSCs, e.g., isolated from bone marrow (BM), peripheral blood (PB), placenta, umbilical cord (UC) or adipose); hematopoietic stem cells (HSCs; e.g. isolated from BM or UC); neural stem cells (NSCs); tissue specific progenitor stem cells (TSPSCs); and limbal stem cells (LSCs). Progenitor and primary cells include mononuclear cells (MNCs, e.g., isolated from BM or PB); endothelial progenitor cells (EPCs, e.g. isolated from BM, PB, and UC); neural progenitor cells (NPCs); and tissue-specific primary cells or cells derived therefrom (TSCs) including chondrocytes, myocytes, and keratinocytes. Cells for organ or tissue transplantations such as islet cells, cardiomyocytes, thyroid cells, thymocytes, neuronal cells, skin cells, and retinal cells are also included.

In some embodiments, the engineered cell is a stem cell.

In some embodiments, the engineered cell is a primary cell.

In some embodiments, the cell is a human cell, such as a cell isolated from a human subject. In some embodiments, the cell is isolated from human donor PBMCs or leukopaks. In some embodiments, the cell is from a subject with a condition, disorder, or disease. In some embodiments, the cell is from a human donor with Epstein Barr Virus (“EBV”).

In some embodiments, the methods are carried out ex vivo. As used herein, “ex vivo” refers to an in vitro method wherein the cell is capable of being transferred into a subject, e.g. as an ACT therapy. In some embodiments, an ex vivo method is an in vitro method involving an ACT therapy cell or cell population.

In some embodiments, the cell is from a cell line. In some embodiments, the cell line is derived from a human subject. In some embodiments, the cell line is a lymphoblastoid cell line (“LCL”). The cell may be cryopreserved and thawed. The cell may not have been previously cryopreserved.

In some embodiments, the cell is from a cell bank. In some embodiments, the cell is genetically modified and then transferred into a cell bank. In some embodiments the cell is removed from a subject, genetically modified ex vivo, and transferred into a cell bank. In some embodiments, a genetically modified population of cells is transferred into a cell bank. In some embodiments, a genetically modified population of immune cells is transferred into a cell bank. In some embodiments, a genetically modified population of immune cells comprising a first and second subpopulations, wherein the first and second sub-populations have at least one common genetic modification and at least one different genetic modification are transferred into a cell bank.

In some embodiments, the present disclosure provides engineered human cells with reduced or eliminated surface expression of HLA-A relative to an unmodified cell, wherein the cell is homozygous for HLA-B and homozygous for HLA-C. The engineered human cells disclosed herein therefore provide a “partial matching” approach to the problem of allogeneic cell transfer and MHC class I compatibility. The use of cells that are homozygous for HLA-B and HLA-C, in addition to reducing or eliminating expression of HLA-A in the cells, limits the number of donors that are necessary to provide a therapy that covers a majority of recipients in population because the disclosed partial matching approach requires only one matching HLA-B allele (as opposed to two) and only one HLA-C allele (as opposed to two). Surprisingly, the engineered human cells that have reduced or eliminated surface expression of HLA-A relative to an unmodified cell, disclosed herein, demonstrate persistence and are protective against NK-mediated rejection, especially as compared to engineered cells with reduced or eliminated B2M expression. The disclosure provides methods and compositions for generating such engineered human cells with reduced or eliminated surface expression of HLA-A relative to an unmodified cell, wherein the cell is homozygous for HLA-B and homozygous for HLA-C. In some embodiments, the disclosure provides engineered human cells, and methods and compositions for generating engineered human cells, wherein the cell further has reduced expression of MHC class II protein on the surface of the cell, e.g., wherein the cell has a genetic modification in the CIITA gene. In some embodiments, the disclosure provides for further engineering of the cell, including to reduce or eliminate the expression of endogenous T-cell receptor proteins (e.g., TRAC), to reduce or eliminate the expression of a protein such as TGFBR2 or CD70, and to introduce an exogenous nucleic acid, e.g., encoding a polypeptide expressed on the cell surface or a polypeptide that is secreted by the cell. Thus, the disclosure thus provides a flexible platform for genetically engineering human cells for a variety of desired adoptive cell therapy purposes.

The present disclosure provides engineered human cells with reduced or eliminated surface expression of HLA-A and HLA-B relative to an unmodified cell, wherein the cell is homozygous for HLA-C. The engineered human cells disclosed herein therefore provide a “partial matching” approach to the problem of allogeneic cell transfer and MHC class I compatibility. The use of cells that are homozygous for HLA-C, in addition to reducing or eliminating expression of HLA-A and HLA-B in the cells, limits the number of donors that are necessary to provide a therapy that covers a majority of recipients in population because the disclosed partial matching approach requires only one matching HLA-C allele (as opposed to two). Surprisingly, the engineered human cells that have reduced or eliminated surface expression of HLA-A and HLA-B relative to an unmodified cell, disclosed herein, demonstrate persistence and are protective against NK-mediated rejection, especially as compared to engineered cells with reduced or eliminated B2M expression. The disclosure provides methods and compositions for generating such engineered human cells with reduced or eliminated surface expression of HLA-A and HLA-B relative to an unmodified cell, wherein the cell is homozygous for HLA-C. In some embodiments, the disclosure provides engineered human cells, and methods and compositions for generating engineered human cells, wherein the cell further has reduced expression of MHC class II protein on the surface of the cell, e.g., wherein the cell has a genetic modification in the CIITA gene. In some embodiments, the disclosure provides for further engineering of the cell, including to reduce or eliminate the expression of endogenous T-cell receptor proteins (e.g., TRAC), to reduce or eliminate the expression of a protein such as TGFBR2 or CD70, and to introduce an exogenous nucleic acid, e.g., encoding a polypeptide expressed on the cell surface or a polypeptide that is secreted by the cell. Thus, the disclosure thus provides a flexible platform for genetically engineering human cells for a variety of desired adoptive cell therapy purposes.

### XII. Therapeutic Methods and Uses

Any of the engineered cells and compositions described herein can be used in a method of treating a variety of diseases and disorders, as described herein. In some embodiments, the genetically modified cell (engineered cell) or population of genetically modified cells (engineered cells) and compositions may be used in methods of treating a variety of diseases and disorders. In some embodiments, a method of treating any one of the diseases or disorders described herein is encompassed, comprising administering any one or more composition described herein.

In some embodiments, the methods and compositions described herein may be used to treat diseases or disorders in need of delivery of a therapeutic agent. In some embodiments, the disclosure provides a method of providing an immunotherapy in a subject, the method including administering to the subject an effective amount of an engineered cell (or population of engineered cells) as described herein, for example, a cell of any of the aforementioned cell aspects and embodiments. In some embodiments, the present disclosure provides for a method of administering the engineered cell, population of cells, or pharmaceutical composition described herein to a subject in need thereof.

In some embodiments, the present disclosure provides for an engineered cell, population of cells, or pharmaceutical composition described herein for use in administration to a subject in need of an adoptive cell transfer (ACT) therapy. In some embodiments, the methods comprise administering to a subject a composition comprising an engineered cell described herein as an adoptive cell transfer therapy. In some embodiments, the engineered cell is an allogeneic cell.

In some embodiments, the present disclosure provides for an engineered cell, population of cells, or pharmaceutical composition described herein for use in treating a subject having a cancer. In some embodiments, the present disclosure provides for an engineered cell, population of cells, or pharmaceutical composition described herein for use in treating a subject having an infectious disease. In some embodiments, the present disclosure provides for an engineered cell, population of cells, or pharmaceutical composition described herein for use in treating a subject having an autoimmune disease. In some embodiments, the present disclosure provides for an engineered cell, population of cells, or pharmaceutical composition described herein for the manufacture of a medicament for the treatment of a subject having a cancer, an infectious disease, or an autoimmune disease. In some embodiments, the cancer is a hematological (heme) malignancy such as, but not limited to, acute myeloid leukemia (AML), Hodgkin's lymphoma, non-Hodgkin's lymphoma, or multiple myeloma. In some embodiments, the hematological malignancy is acute myeloid leukemia. In some embodiments, the hematological malignancy is multiple myeloma. In some embodiments, the cancer is a solid tumor such as, but not limited to, renal cell carcinoma (RCC), thymic carcinoma, pancreatic carcinoma, nasopharyngeal carcinoma, ovarian carcinoma, melanoma, brain carcinoma, colon carcinoma, lung carcinoma, or breast carcinoma. In some embodiments, the solid tumor is renal cell carcinoma.

In some embodiments of the methods, the method includes administering a lymphodepleting agent or immunosuppressant prior to administering to the subject an effective amount of the engineered cell (or engineered cells) as described herein, for example, a cell of any of the aforementioned cell aspects and embodiments. In another aspect, the disclosure provides a method of preparing engineered cells (e.g., a population of engineered cells).

Immunotherapy is the treatment of disease by activating or suppressing the immune system. Immunotherapies designed to elicit or amplify an immune response are classified as activation immunotherapies. Cell-based immunotherapies have been demonstrated to be effective in the treatment of some cancers. Immune effector cells such as lymphocytes, macrophages, dendritic cells, natural killer cells, cytotoxic T lymphocytes (CTLs), T helper cells, B cells, or their progenitors such as hematopoietic stem cells (HSC) or induced pluripotent stem cells (iPSC) can be programmed to act in response to abnormal antigens expressed on the surface of tumor cells. Thus, cancer immunotherapy allows components of the immune system to destroy tumors or other cancerous cells. Cell-based immunotherapies have also been demonstrated to be effective in the treatment of autoimmune diseases or transplant rejection. Immune effector cells such as regulatory T-cells (Tregs) or mesenchymal stem cells can be programmed to act in response to autoantigens or transplant antigens expressed on the surface of normal tissues.

In some embodiments, the disclosure provides a method of preparing engineered cells (e.g., a population of engineered cells). The population of engineered cells may be used for immunotherapy.

In some embodiments, the disclosure provides a method of treating a subject in need thereof that includes administering engineered cells prepared by a method of preparing cells described herein, for example, a method of any of the aforementioned aspects and embodiments of methods of preparing cells.

In some embodiments, the engineered cells can be used to treat cancer, infectious diseases, inflammatory diseases, autoimmune diseases, cardiovascular diseases, neurological diseases, ophthalmologic diseases, renal diseases, liver diseases, musculoskeletal diseases, red blood cell diseases, or transplant rejections. In some embodiments, the engineered cells can be used in cell transplant, e.g., to the heart, liver, lung, kidney, pancreas, skin, or brain. (See e.g., Deuse et al., Nature Biotechnology 37:252-258 (2019).)

In some embodiments, the engineered cells can be used as a cell therapy comprising an allogeneic stem cell therapy. In some embodiments, the cell therapy comprises induced pluripotent stem cells (iPSCs). iPSCs may be induced to differentiate into other cell types including e.g., beta islet cells, neurons, and blood cells. In some embodiments, the cell therapy comprises hematopoietic stem cells. In some embodiments, the stem cells comprise mesenchymal stem cells that can develop into bone, cartilage, muscle, and fat cells. In some embodiments, the stem cells comprise ocular stem cells. In some embodiments, the allogeneic stem cell transplant comprises allogeneic bone marrow transplant. In some embodiments, the stem cells comprise pluripotent stem cells (PSCs). In some embodiments, the stem cells comprise induced embryonic stem cells (ESCs).

In some embodiments, the methods provide for administering the engineered cells to a subject, wherein the administration is an injection. In some embodiments, the methods provide for administering the engineered cells to a subject, wherein the administration is an intravascular injection or infusion. In some embodiments, the methods provide for administering the engineered cells to a subject, wherein the administration is a single dose.

In some embodiments, the methods provide for reducing a sign or symptom associated of a subject's disease treated with a composition disclosed herein. In some embodiments, the subject has a response to treatment with a composition disclosed herein that lasts more than one week. In some embodiments, the subject has a response to treatment with a composition disclosed herein that lasts more than two weeks. In some embodiments, the subject has a response to treatment with a composition disclosed herein that lasts more than three weeks. In some embodiments, the subject has a response to treatment with a composition disclosed herein that lasts more than one month.

In some embodiments, the methods provide for administering the engineered cells to a subject, and wherein the subject has a response to the administered cell that comprises a reduction in a sign or symptom associated with the disease treated by the cell therapy. In some embodiments, the subject has a response that lasts more than one week. In some embodiments, the subject has a response that lasts more than one month. In some embodiments, the subject has a response that lasts for at least 1-6 weeks.

TABLE 10
Additional Sequences

| SEQ ID NO | Description | SEQUENCE |
|---|---|---|
| 801 | Exemplary ORF for Nme2Cas9 base editor | ATGGACGGCTCCGGCGGCGGCTCCCCCAAGAAGAAGCGGAAGGTGGAGGACAAGCGGCCCGCCGCCACCAAGAAGGCCGGCCAGGCCA |
| | | AGAAGAAGAAGGGCGGCTCCGGCGGCGGCGAGGCCTCCCCCGCCTCCGGCCCCCGGCACCTGATGGACCCCCACATCTTCACCTCCAA |
| | | CTTCAACAACGGCATCGGCCGGCACAAGACCTACCTGTGCTACGAGGTGGAGCGGCTGGACAACGGCACCTCCGTGAAGATGGACCAG |
| | | CACCGGGGCTTCCTGCACAACCAGGCCAAGAACCTGCTGTGCGGCTTCTACGGCCGGCACGCCGAGCTGCGGTTCCTGGACCTGGTGC |
| | | CCTCCCTGCAGCTGGACCCCGCCCAGATCTACCGGGTGACCTGGTTCATCTCCTGGTCCCCCTGCTTCTCCTGGGGCTGCGCCGGCGA |
| | | GGTGCGGGCCTTCCTGCAGGAGAACACCCACGTGCGGCTGCGGATCTTCGCCGCCCGGATCTACGACTACGACCCCCTGTACAAGGAG |
| | | GCCCTGCAGATGCTGCGGGACGCCGGCGCCCAGGTGTCCATCATGACCTACGACGAGTTCAAGCACTGCTGGGACACCTTCGTGGACC |
| | | ACCAGGGCTGCCCCTTCCAGCCCTGGGACGGCCTGGACGAGCACTCCCAGGCCCTGTCCGGCCGGCTGCGGGCCATCCTGCAGAACCA |
| | | GGGCAACTCCGGCTCCGAGACCCCCGGCACCTCCGAGTCCGCCACCCCCGAGTCCGCAGCGTTCAAACCAAATCCCATCAACTACATC |
| | | CTGGGCCTGGCCATCGGCATCGCCTCCGTGGGCTGGGCCATGGTGGAGATCGACGAGGAGGAGAACCCCATCCGGCTGATCGACCTGG |
| | | GCGTGCGGGTGTTCGAGCGGGCCGAGGTGCCCAAGACCGGCGACTCCCTGGCCATGGCCCGGCGGCTGGCCCGGTCCGTGCGGCGGCT |
| | | GACCCGGCGGCGGGCCCACCGGCTGCTGCGGGCCCGGCGGCTGCTGAAGCGGGAGGGCGTGCTGCAGGCCGCCGACTTCGACGAGAAC |
| | | GGCCTGATCAAGTCCCTGCCCAACACCCCCTGGCAGCTGCGGGCCGCCGCCCTGGACCGGAAGCTGACCCCCCTGGAGTGGTCCGCCG |
| | | TGCTGCTGCACCTGATCAAGCACCGGGGCTACCTGTCCCAGCGGAAGAACGAGGGCGAGACCGCCGACAAGGAGCTGGGCGCCCTGCT |
| | | GAAGGGCGTGGCCAACAACGCCCACGCCCTGCAGACCGGCGACTTCCGGACCCCCGCCGAGCTGGCCCTGAACAAGTTCGAGAAGGAG |
| | | TCCGGCCACATCCGGAACCAGCGGGGCGACTACTCCCACACCTTCTCCCGGAAGGACCTGCAGGCCGAGCTGATCCTGCTGTTCGAGA |
| | | AGCAGAAGGAGTTCGGCAACCCCCACGTGTCCGGCGGCCTGAAGGAGGGCATCGAGACCCTGCTGATGACCCAGCGGCCCGCCCTGTC |
| | | CGGCGACGCCGTGCAGAAGATGCTGGGCCACTGCACCTTCGAGCCCGCCGAGCCCAAGGCCGCCAAGAACACCTACACCGCCGAGCGG |
| | | TTCATCTGGCTGACCAAGCTGAACAACCTGCGGATCCTGGAGCAGGGCTCCGAGCGGCCCCTGACCGACACCGAGCGGGCCACCCTGA |
| | | TGGACGAGCCCTACCGGAAGTCCAAGCTGACCTACGCCCAGGCCCGGAAGCTGCTGGGCCTGGAGGACACCGCCTTCTTCAAGGGCCT |
| | | GCGGTACGGCAAGGACAACGCCGAGGCCTCCACCCTGATGGAGATGAAGGCCTACCACGCCATCTCCCGGGCCCTGGAGAAGGAGGGC |
| | | CTGAAGGACAAGAAGTCCCCCCTGAACCTGTCCTCCGAGCTGCAGGACGAGATCGGCACCGCCTTCTCCCTGTTCAAGACCGACGAGG |
| | | ACATCACCGGCCGGCTGAAGGACCGGGTGCAGCCCGAGATCCTGGAGGCCCTGCTGAAGCACATCTCCTTCGACAAGTTCGTGCAGAT |
| | | CTCCCTGAAGGCCCTGCGGCGGATCGTGCCCCTGATGGAGCAGGGCAAGCGGTACGACGAGGCCTGCGCCGAGATCTACGGCGACCAC |
| | | TACGGCAAGAAGAACACCGAGGAGAAGATCTACCTGCCCCCCATCCCCGCCGACGAGATCCGGAACCCCGTGGTGCTGCGGGCCCTGT |
| | | CCCAGGCCCGGAAGGTGATCAACGGCGTGGTGCGGCGGTACGGCTCCCCCGCCCGGATCCACATCGAGACCGCCCGGGAGGTGGGCAA |
| | | GTCCTTCAAGGACCGGAAGGAGATCGAGAAGCGGCAGGAGGAGAACCGGAAGGACCGGGAGAAGGCCGCCGCCAAGTTCCGGGAGTAC |
| | | TTCCCCAACTTCGTGGGCGAGCCCAAGTCCAAGGACATCCTGAAGCTGCGGCTGTACGAGCAGCAGCACGGCAAGTGCCTGTACTCCG |
| | | GCAAGGAGATCAACCTGGTGCGGCTGAACGAGAAGGGCTACGTGGAGATCGACCACGCCCTGCCCTTCTCCCGGACCTGGGACGACTC |
| | | CTTCAACAACAAGGTGCTGGTGCTGGGCTCCGAGAACCAGAACAAGGGCAACCAGACCCCCTACGAGTACTTCAACGGCAAGGACAAC |
| | | TCCCGGGAGTGGCAGGAGTTCAAGGCCCGGGTGGAGACCTCCCGGTTCCCCCGGTCCAAGAAGCAGCGGATCCTGCTGCAGAAGTTCG |
| | | ACGAGGACGGCTTCAAGGAGTGCAACCTGAACGACACCCGGTACGTGAACCGCTTCCTGTGCCAGTTCGTGGCCGACCACATCCTGCT |
| | | GACCGGCAAGGGCAAGCGGCGGGTGTTCGCCTCCAACGGCCAGATCACCAACCTGCTGCGGGGCTTCTGGGGCCTGCGGAAGGTGCGG |
| | | GCCGAGAACGACCGGCACCACGCCCTGGACGCCGTGGTGGTGGCCTGCTCCACCGTGGCCATGCAGCAGAAGATCACCCGGTTCGTGC |
| | | GGTACAAGGAGATGAACGCCTTCGACGGCAAGACCATCGACAAGGAGACCGGCAAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCCCCGAG |
| | | AAGCTGCGGACCCTGCTGGCCGAGAAGCTGTCCTCCCGGCCCGAGGCCGTGCACGAGTACGTGACCCCCCTGTTCGTGTCCCGGGCCC |
| | | CCAACCGGAAGATGTCCGGCGCCCACAAGGACACCCTGCGGTCCGCCAAGCGGTTCGTGAAGCACAACGAGAAGATCTCCGTGAAGCG |
| | | GGTGTGGCTGACCGAGATCAAGCTGGCCGACCTGGAGAACATGGTGAACTACAAGAACGGCCGGGAGATCGAGCTGTACGAGGCCCTG |
| | | AAGGCCCGGCTGGAGGCCTACGGCGGCAACGCCAAGCAGGCCTTCGACCCCAAGGACAACCCCTTCTACAAGAAGGGCGGCCAGCTGG |
| | | TGAAGGCCGTGCGGGTGGAGAAGACCCAGGAGTCCGGCGTGCTGCTGAACAAGAAGAACGCCTACACCATCGCCGACAACGGCGACAT |
| | | GGTGCGGGTGGACGTGTTCTGCAAGGTGGACAAGAAGGGCAAGAACCAGTACTTCATCGTGCCCATCTACGCCTGGCAGGTGGCCGAG |
| | | AACATCCTGCCCGACATCGACTGCAAGGGCTACCGGATCGACGACTCCTACACCTTCTGCTTCTCCCTGCACAAGTACGACCTGATCG |
| | | CCTTCCAGAAGGACGAGAAGTCCAAGGTGGAGTTCGCCTACTACATCAACTGCGACTCCTCCAACGGCCGGTTCTACCTGGCCTGGCA |
| | | CGACAAGGGCTCCAAGGAGCAGCAGTTCCGGATCTCCACCCAGAACCTGGTGCTGATCCAGAAGTACCAGGTGAACGAGCTGGGCAAG |
| | | GAGATCCGGCCCTGCCGGCTGAAGAAGCGGCCCCCCGTGCGGTAG |
| 802 | ORF encoding Nme2 Cas9 | ATGGACGGCTCCGGCGGCGGCTCCCCCAAGAAGAAGCGGAAGGTGGAGGACAAGCGGCCCGCCGCCACCAAGAAGGCCGGCCAGGCCA |
| | | AGAAGAAGAAGGGCGGCTCCGGCGGCGGCGCCGCCTTCAAGCCCAACCCCATCAACTACATCCTGGGCCTGGACATCGGCATCGCCTC |
| | | CGTGGGCTGGGCCATGGTGGAGATCGACGAGGAGGAGAACCCCATCCGGCTGATCGACCTGGGCGTGCGGGTGTTCGAGCGGGCCGAG |
| | | GTGCCCAAGACCGGCGACTCCCTGGCCATGGCCCGGCGGCTGGCCCGGTCCGTGCGGCGGCTGACCCGGCGGCGGGCCCACCGGCTGC |
| | | TGCGGGCCCGGCGGCTGCTGAAGCGGGAGGGCGTGCTGCAGGCCGCCGACTTCGACGAGAACGGCCTGATCAAGTCCCTGCCCAACAC |
| | | CCCCTGGCAGCTGCGGGCCGCCGCCCTGGACCGGAAGCTGACCCCCCTGGAGTGGTCCGCCGTGCTGCTGCACCTGATCAAGCACCGG |
| | | GGCTACCTGTCCCAGCGGAAGAACGAGGGCGAGACCGCCGACAAGGAGCTGGGCGCCCTGCTGAAGGGCGTGGCCAACAACGCCCACG |
| | | CCCTGCAGACCGGCGACTTCCGGACCCCCGCCGAGCTGGCCCTGAACAAGTTCGAGAAGGAGTCCGGCCACATCCGGAACCAGCGGGG |
| | | CGACTACTCCCACACCTTCTCCCGGAAGGACCTGCAGGCCGAGCTGATCCTGCTGTTCGAGAAGCAGAAGGAGTTCGGCAACCCCCAC |
| | | GTGTCCGGCGGCCTGAAGGAGGGCATCGAGACCCTGCTGATGACCCAGCGGCCCGCCCTGTCCGGCGACGCCGTGCAGAAGATGCTGG |
| | | GCCACTGCACCTTCGAGCCCGCCGAGCCCAAGGCCGCCAAGAACACCTACACCGCCGAGCGGTTCATCTGGCTGACCAAGCTGAACAA |
| | | CCTGCGGATCCTGGAGCAGGGCTCCGAGCGGCCCCTGACCGACACCGAGCGGGCCACCCTGATGGACGAGCCCTACCGGAAGTCCAAG |
| | | CTGACCTACGCCCAGGCCCGGAAGCTGCTGGGCCTGGAGGACACCGCCTTCTTCAAGGGCCTGCGGTACGGCAAGGACAACGCCGAGG |
| | | CCTCCACCCTGATGGAGATGAAGGCCTACCACGCCATCTCCCGGGCCCTGGAGAAGGAGGGCCTGAAGGACAAGAAGTCCCCCCTGAA |
| | | CCTGTCCTCCGAGCTGCAGGACGAGATCGGCACCGCCTTCTCCCTGTTCAAGACCGACGAGGACATCACCGGCCGGCTGAAGGACCGG |
| | | GTGCAGCCCGAGATCCTGGAGGCCCTGCTGAAGCACATCTCCTTCGACAAGTTCGTGCAGATCTCCCTGAAGGCCCTGCG |
| | | GCGGATCGTGCCCCTGATGGAGCAGGGCAAGCGGTACGACGAGGCCTGCG |
| | | CCGAGATCTACGGCGACCACTACGGCAAGAAGAACACCGAGGAGAAGATC |
| | | TACCTGCCCCCCATCCCCGCCGACGAGATCCGGAACCCCGTGGTGCTGCG |
| | | GGCCCTGTCCCAGGCCCGGAAGGTGATCAACGGCGTGGTGCGGCGGTACG |
| | | GCTCCCCCGCCCGGATCCACATCGAGACCGCCCGGGAGGTGGGCAAGTCC |
| | | TTCAAGGACCGGAAGGAGATCGAGAAGCGGCAGGAGGAGAACCGGAAGGA |
| | | CCGGGAGAAGGCCGCCGCCAAGTTCCGGGAGTACTTCCCCAACTTCGTGG |
| | | GCGAGCCCAAGTCCAAGGACATCCTGAAGCTGCGGCTGTACGAGCAGCAG |
| | | CACGGCAAGTGCCTGTACTCCGGCAAGGAGATCAACCTGGTGCGGCTGAA |
| | | CGAGAAGGGCTACGTGGAGATCGACCACGCCCTGCCCTTCTCCCGGACCT |
| | | GGGACGACTCCTTCAACAACAAGGTGCTGGTGCTGGGCTCCGAGAACCAG |
| | | AACAAGGGCAACCAGACCCCCTACGAGTACTTCAACGGCAAGGACAACTC |
| | | CCGGGAGTGGCAGGAGTTCAAGGCCCGGGTGGAGACCTCCCGGTTCCCCC |
| | | GGTCCAAGAAGCAGCGGATCCTGCTGCAGAAGTTCGACGAGGACGGCTTC |
| | | AAGGAGTGCAACCTGAACGACACCCGGTACGTGAACCGGTTCCTGTGCCA |
| | | GTTCGTGGCCGACCACATCCTGCTGACCGGCAAGGGCAAGCGGCGGGTGT |
| | | TCGCCTCCAACGGCCAGATCACCAACCTGCTGCGGGGCTTCTGGGGCCTG |
| | | CGGAAGGTGCGGGCCGAGAACGACCGGCACCACGCCCTGGACGCCGTGGT |
| | | GGTGGCCTGCTCCACCGTGGCCATGCAGCAGAAGATCACCCGGTTCGTGC |
| | | GGTACAAGGAGATGAACGCCTTCGACGGCAAGACCATCGACAAGGAGACC |
| | | GGCAAGGTGCTGCACCAGAAGACCCACTTCCCCCAGCCCTGGGAGTTCTT |
| | | CGCCCAGGAGGTGATGATCCGGGTGTTCGGCAAGCCCGACGGCAAGCCCG |
| | | AGTTCGAGGAGGCCGACACCCCCGAGAAGCTGCGGACCCTGCTGGCCGAG |
| | | AAGCTGTCCTCCCGGCCCGAGGCCGTGCACGAGTACGTGACCCCCCTGTT |
| | | CGTGTCCCGGGCCCCCAACCGGAAGATGTCCGGCGCCCACAAGGACACCC |
| | | TGCGGTCCGCCAAGCGGTTCGTGAAGCACAACGAGAAGATCTCCGTGAAG |
| | | CGGGTGTGGCTGACCGAGATCAAGCTGGCCGACCTGGAGAACATGGTGAA |
| | | CTACAAGAACGGCCGGGAGATCGAGCTGTACGAGGCCCTGAAGGCCCGGC |
| | | TGGAGGCCTACGGCGGCAACGCCAAGCAGGCCTTCGACCCCAAGGACAAC |
| | | CCCTTCTACAAGAAGGGCGGCCAGCTGGTGAAGGCCGTGCGGGTGGAGAA |
| | | GACCCAGGAGTCCGGCGTGCTGCTGAACAAGAAGAACGCCTACACCATCG |
| | | CCGACAACGGCGACATGGTGCGGGTGGACGTGTTCTGCAAGGTGGACAAG |
| | | AAGGGCAAGAACCAGTACTTCATCGTGCCCATCTACGCCTGGCAGGTGGC |
| | | CGAGAACATCCTGCCCGACATCGACTGCAAGGGCTACCGGATCGACGACT |
| | | CCTACACCTTCTGCTTCTCCCTGCACAAGTACGACCTGATCGCCTTCCAG |
| | | AAGGACGAGAAGTCCAAGGTGGAGTTCGCCTACTACATCAACTGCGACTC |
| | | CTCCAACGGCCGGTTCTACCTGGCCTGGCACGACAAGGGCTCCAAGGAGC |
| | | AGCAGTTCCGGATCTCCACCCAGAACCTGGTGCTGATCCAGAAGTACCAG |
| | | GTGAACGAGCTGGGCAAGGAGATCCGGCCCTGCCGGCTGAAGAAGCGGCC |
| | | CCCCGTGCGGTAG |
| 803 | ORF encoding Nme2 Cas9 with Hibit Tab | ATGGACGGCTCCGGCGGCGGCTCCCCCAAGAAGAAGCGGAAGGTGGAGGA |
| | | CAAGCGGCCCGCCGCCACCAAGAAGGCCGGCCAGGCCAAGAAGAAGAAGG |
| | | GCGGCTCCGGCGGCGGCGCCGCCTTCAAGCCCAACCCCATCAACTACATC |
| | | CTGGGCCTGGACATCGGCATCGCCTCCGTGGGCTGGGCCATGGTGGAGAT |
| | | CGACGAGGAGGAGAACCCCATCCGGCTGATCGACCTGGGCGTGCGGGTGT |
| | | TCGAGCGGGCCGAGGTGCCCAAGACCGGCGACTCCCTGGCCATGGCCCGG |
| | | CGGCTGGCCCGGTCCGTGCGGCGGCTGACCCGGCGGCGGGCCCACCGGCT |
| | | GCTGCGGGCCCGGCGGCTGCTGAAGCGGGAGGGCGTGCTGCAGGCCGCCG |
| | | ACTTCGACGAGAACGGCCTGATCAAGTCCCTGCCCAACACCCCCTGGCAG |
| | | CTGCGGGCCGCCGCCCTGGACCGGAAGCTGACCCCCCTGGAGTGGTCCGC |
| | | CGTGCTGCTGCACCTGATCAAGCACCGGGGCTACCTGTCCCAGCGGAAGA |
| | | ACGAGGGCGAGACCGCCGACAAGGAGCTGGGCGCCCTGCTGAAGGGCGTG |
| | | GCCAACAACGCCCACGCCCTGCAGACCGGCGACTTCCGGACCCCCGCCGA |
| | | GCTGGCCCTGAACAAGTTCGAGAAGGAGTCCGGCCACATCCGGAACCAGC |
| | | GGGGCGACTACTCCCACACCTTCTCCCGGAAGGACCTGCAGGCCGAGCTG |
| | | ATCCTGCTGTTCGAGAAGCAGAAGGAGTTCGGCAACCCCCACGTGTCCGG |
| | | CGGCCTGAAGGAGGGCATCGAGACCCTGCTGATGACCCAGCGGCCCGCCC |
| | | TGTCCGGCGACGCCGTGCAGAAGATGCTGGGCCACTGCACCTTCGAGCCC |
| | | GCCGAGCCCAAGGCCGCCAAGAACACCTACACCGCCGAGCGGTTCATCTG |
| | | GCTGACCAAGCTGAACAACCTGCGGATCCTGGAGCAGGGCTCCGAGCGGC |
| | | CCCTGACCGACACCGAGCGGGCCACCCTGATGGACGAGCCCTACCGGAAG |
| | | TCCAAGCTGACCTACGCCCAGGCCCGGAAGCTGCTGGGCCTGGAGGACAC |
| | | CGCCTTCTTCAAGGGCCTGCGGTACGGCAAGGACAACGCCGAGGCCTCCA |
| | | CCCTGATGGAGATGAAGGCCTACCACGCCATCTCCCGGGCCCTGGAGAAG |
| | | GAGGGCCTGAAGGACAAGAAGTCCCCCCTGAACCTGTCCTCCGAGCTGCA |
| | | GGACGAGATCGGCACCGCCTTCTCCCTGTTCAAGACCGACGAGGACATCA |
| | | CCGGCCGGCTGAAGGACCGGGTGCAGCCCGAGATCCTGGAGGCCCTGCTG |
| | | AAGCACATCTCCTTCGACAAGTTCGTGCAGATCTCCCTGAAGGCCCTGCG |
| | | GCGGATCGTGCCCCTGATGGAGCAGGGCAAGCGGTACGACGAGGCCTGCG |
| | | CCGAGATCTACGGCGACCACTACGGCAAGAAGAACACCGAGGAGAAGATC |
| | | TACCTGCCCCCCATCCCCGCCGACGAGATCCGGAACCCCGTGGTGCTGCG |
| | | GGCCCTGTCCCAGGCCCGGAAGGTGATCAACGGCGTGGTGCGGCGGTACG |
| | | GCTCCCCCGCCCGGATCCACATCGAGACCGCCCGGGAGGTGGGCAAGTCC |
| | | TTCAAGGACCGGAAGGAGATCGAGAAGCGGCAGGAGGAGAACCGGAAGGA |
| | | CCGGGAGAAGGCCGCCGCCAAGTTCCGGGAGTACTTCCCCAACTTCGTGG |
| | | GCGAGCCCAAGTCCAAGGACATCCTGAAGCTGCGGCTGTACGAGCAGCAG |
| | | CACGGCAAGTGCCTGTACTCCGGCAAGGAGATCAACCTGGTGCGGCTGAA |
| | | CGAGAAGGGCTACGTGGAGATCGACCACGCCCTGCCCTTCTCCCGGACCT |
| | | GGGACGACTCCTTCAACAACAAGGTGCTGGTGCTGGGCTCCGAGAACCAG |
| | | AACAAGGGCAACCAGACCCCCTACGAGTACTTCAACGGCAAGGACAACTC |
| | | CCGGGAGTGGCAGGAGTTCAAGGCCCGGGTGGAGACCTCCCGGTTCCCCC |
| | | GGTCCAAGAAGCAGCGGATCCTGCTGCAGAAGTTCGACGAGGACGGCTTC |
| | | AAGGAGTGCAACCTGAACGACACCCGGTACGTGAACCGGTTCCTGTGCCA |
| | | GTTCGTGGCCGACCACATCCTGCTGACCGGCAAGGGCAAGCGGCGGGTGT |
| | | TCGCCTCCAACGGCCAGATCACCAACCTGCTGCGGGGCTTCTGGGGCCTG |
| | | CGGAAGGTGCGGGCCGAGAACGACCGGCACCACGCCCTGGACGCCGTGGT |
| | | GGTGGCCTGCTCCACCGTGGCCATGCAGCAGAAGATCACCCGGTTCGTGC |
| | | GGTACAAGGAGATGAACGCCTTCGACGGCAAGACCATCGACAAGGAGACC |
| | | GGCAAGGTGCTGCACCAGAAGACCCACTTCCCCCAGCCCTGGGAGTTCTT |
| | | CGCCCAGGAGGTGATGATCCGGGTGTTCGGCAAGCCCGACGGCAAGCCCG |
| | | AGTTCGAGGAGGCCGACACCCCCGAGAAGCTGCGGACCCTGCTGGCCGAG |
| | | AAGCTGTCCTCCCGGCCCGAGGCCGTGCACGAGTACGTGACCCCCCTGTT |
| | | CGTGTCCCGGGCCCCCAACCGGAAGATGTCCGGCGCCCACAAGGACACCC |
| | | TGCGGTCCGCCAAGCGGTTCGTGAAGCACAACGAGAAGATCTCCGTGAAG |
| | | CGGGTGTGGCTGACCGAGATCAAGCTGGCCGACCTGGAGAACATGGTGAA |
| | | CTACAAGAACGGCCGGGAGATCGAGCTGTACGAGGCCCTGAAGGCCCGGC |
| | | TGGAGGCCTACGGCGGCAACGCCAAGCAGGCCTTCGACCCCAAGGACAAC |
| | | CCCTTCTACAAGAAGGGCGGCCAGCTGGTGAAGGCCGTGCGGGTGGAGAA |
| | | GACCCAGGAGTCCGGCGTGCTGCTGAACAAGAAGAACGCCTACACCATCG |
| | | CCGACAACGGCGACATGGTGCGGGTGGACGTGTTCTGCAAGGTGGACAAG |
| | | AAGGGCAAGAACCAGTACTTCATCGTGCCCATCTACGCCTGGCAGGTGGC |
| | | CGAGAACATCCTGCCCGACATCGACTGCAAGGGCTACCGGATCGACGACT |
| | | CCTACACCTTCTGCTTCTCCCTGCACAAGTACGACCTGATCGCCTTCCAG |
| | | AAGGACGAGAAGTCCAAGGTGGAGTTCGCCTACTACATCAACTGCGACTC |
| | | CTCCAACGGCCGGTTCTACCTGGCCTGGCACGACAAGGGCTCCAAGGAGC |
| | | AGCAGTTCCGGATCTCCACCCAGAACCTGGTGCTGATCCAGAAGTACCAG |
| | | GTGAACGAGCTGGGCAAGGAGATCCGGCCCTGCCGGCTGAAGAAGCGGCC |
| | | CCCCGTGCGGTCCGAGTCCGCCACCCCCGAGTCCGTGTCCGGCTGGCGGC |
| | | TGTTCAAGAAGATCTCCTAG |
| 804 | Exemplary ORF encoding Nme2 base editor | ATGGACGGCTCCGGCGGCGGCTCCCCCAAGAAGAAGCGGAAGGTGGAGGACAAGCGGCCCGCCGCCACCAAGAAGGCCGGCCAGGCCA |
| | | AGAAGAAGAAGGGCGGCTCCGGCGGCGGCGAGGCCTCCCCCGCCTCCGGCCCCCGGCACCTGATGGACCCCCACATCTTCACCTCCAA |
| | | CTTCAACAACGGCATCGGCCGGCACAAGACCTACCTGTGCTACGAGGTGGAGCGGCTGGACAACGGCACCTCCGTGAAGATGGACCAG |
| | | CACCGGGGCTTCCTGCACAACCAGGCCAAGAACCTGCTGTGCGGCTTCTACGGCCGGCACGCCGAGCTGCGGTTCCTGGACCTGGTGC |
| | | CCTCCCTGCAGCTGGACCCCGCCCAGATCTACCGGGTGACCTGGTTCATCTCCTGGTCCCCCTGCTTCTCCTGGGGCTGCGCCGGCGA |
| | | GGTGCGGGCCTTCCTGCAGGAGAACACCCACGTGCGGCTGCGGATCTTCGCCGCCCGGATCTACGACTACGACCCCCTGTACAAGGAG |
| | | GCCCTGCAGATGCTGCGGGACGCCGGCGCCCAGGTGTCCATCATGACCTACGACGAGTTCAAGCACTGCTGGGACACCTTCGTGGACC |
| | | ACCAGGGCTGCCCCTTCCAGCCCTGGGACGGCCTGGACGAGCACTCCCAGGCCCTGTCCGGCCGGCTGCGGGCCATCCTGCAGAACCA |
| | | GGGCAACGGCACCAAGGACTCCACCAAGGACATCCCCGAGACCCCCTCCAAGGACGCAGCGTTCAAACCAAATCCCATCAACTACATC |
| | | CTGGGCCTGGCCATCGGCATCGCCTCCGTGGGCTGGGCCATGGTGGAGATCGACGAGGAGGAGAACCCCATCCGGCTGATCGACCTGG |
| | | GCGTGCGGGTGTTCGAGCGGGCCGAGGTGCCCAAGACCGGCGACTCCCTGGCCATGGCCCGGCGGCTGGCCCGGTCCGTGCGGCGGCT |
| | | GACCCGGCGGCGGGCCCACCGGCTGCTGCGGGCCCGGCGGCTGCTGAAGCGGGAGGGCGTGCTGCAGGCCGCCGACTTCGACGAGAAC |
| | | GGCCTGATCAAGTCCCTGCCCAACACCCCCTGGCAGCTGCGGGCCGCCGCCCTGGACCGGAAGCTGACCCCCCTGGAGTGGTCCGCCG |
| | | TGCTGCTGCACCTGATCAAGCACCGGGGCTACCTGTCCCAGCGGAAGAACGAGGGCGAGACCGCCGACAAGGAGCTGGGCGCCCTGCT |
| | | GAAGGGCGTGGCCAACAACGCCCACGCCCTGCAGACCGGCGACTTCCGGACCCCCGCCGAGCTGGCCCTGAACAAGTTCGAGAAGGAG |
| | | TCCGGCCACATCCGGAACCAGCGGGGCGACTACTCCCACACCTTCTCCCGGAAGGACCTGCAGGCCGAGCTGATCCTGCTGTTCGAGA |
| | | AGCAGAAGGAGTTCGGCAACCCCCACGTGTCCGGCGGCCTGAAGGAGGGCATCGAGACCCTGCTGATGACCCAGCGGCCCGCCCTGTC |
| | | CGGCGACGCCGTGCAGAAGATGCTGGGCCACTGCACCTTCGAGCCCGCCGAGCCCAAGGCCGCCAAGAACACCTACACCGCCGAGCGG |
| | | TTCATCTGGCTGACCAAGCTGAACAACCTGCGGATCCTGGAGCAGGGCTCCGAGCGGCCCCTGACCGACACCGAGCGGGCCACCCTGA |
| | | TGGACGAGCCCTACCGGAAGTCCAAGCTGACCTACGCCCAGGCCCGGAAGCTGCTGGGCCTGGAGGACACCGCCTTCTTCAAGGGCCT |
| | | GCGGTACGGCAAGGACAACGCCGAGGCCTCCACCCTGATGGAGATGAAGGCCTACCACGCCATCTCCCGGGCCCTGGAGAAGGAGGGC |
| | | CTGAAGGACAAGAAGTCCCCCCTGAACCTGTCCTCCGAGCTGCAGGACGAGATCGGCACCGCCTTCTCCCTGTTCAAGACCGACGAGG |
| | | ACATCACCGGCCGGCTGAAGGACCGGGTGCAGCCCGAGATCCTGGAGGCCCTGCTGAAGCACATCTCCTTCGACAAGTTCGTGCAGAT |
| | | CTCCCTGAAGGCCCTGCGGCGGATCGTGCCCCTGATGGAGCAGGGCAAGCGGTACGACGAGGCCTGCGCCGAGATCTACGGCGACCAC |
| | | TACGGCAAGAAGAACACCGAGGAGAAGATCTACCTGCCCCCCATCCCCGCCGACGAGATCCGGAACCCCGTGGTGCTGCGGGCCCTGT |
| | | CCCAGGCCCGGAAGGTGATCAACGGCGTGGTGCGGCGGTACGGCTCCCCCGCCCGGATCCACATCGAGACCGCCCGGGAGGTGGGCAA |
| | | GTCCTTCAAGGACCGGAAGGAGATCGAGAAGCGGCAGGAGGAGAACCGGAAGGACCGGGAGAAGGCCGCCGCCAAGTTCCGGGAGTAC |
| | | TTCCCCAACTTCGTGGGCGAGCCCAAGTCCAAGGACATCCTGAAGCTGCGGCTGTACGAGCAGCAGCACGGCAAGTGCCTGTACTCCG |
| | | GCAAGGAGATCAACCTGGTGCGGCTGAACGAGAAGGGCTACGTGGAGATCGACCACGCCCTGCCCTTCTCCCGGACCTGGGACGACTC |
| | | CTTCAACAACAAGGTGCTGGTGCTGGGCTCCGAGAACCAGAACAAGGGCAACCAGACCCCCTACGAGTACTTCAACGGCAAGGACAAC |
| | | TCCCGGGAGTGGCAGGAGTTCAAGGCCCGGGTGGAGACCTCCCGGTTCCCCCGGTCCAAGAAGCAGCGGATCCTGCTGCAGAAGTTCG |
| | | ACGAGGACGGCTTCAAGGAGTGCAACCTGAACGACACCCGGTACGTGAACCGCTTCCTGTGCCAGTTCGTGGCCGACCACATCCTGCT |
| | | GACCGGCAAGGGCAAGCGGCGGGTGTTCGCCTCCAACGGCCAGATCACCAACCTGCTGCGGGGCTTCTGGGGCCTGCGGAAGGTGCGG |
| | | GCCGAGAACGACCGGCACCACGCCCTGGACGCCGTGGTGGTGGCCTGCTCCACCGTGGCCATGCAGCAGAAGATCACCCGGTTCGTGC |
| | | GGTACAAGGAGATGAACGCCTTCGACGGCAAGACCATCGACAAGGAGACCGGCAAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCCCCGAG |
| | | AAGCTGCGGACCCTGCTGGCCGAGAAGCTGTCCTCCCGGCCCGAGGCCGTGCACGAGTACGTGACCCCCCTGTTCGTGTCCCGGGCCC |
| | | CCAACCGGAAGATGTCCGGCGCCCACAAGGACACCCTGCGGTCCGCCAAGCGGTTCGTGAAGCACAACGAGAAGATCTCCGTGAAGCG |
| | | GGTGTGGCTGACCGAGATCAAGCTGGCCGACCTGGAGAACATGGTGAACTACAAGAACGGCCGGGAGATCGAGCTGTACGAGGCCCTG |
| | | AAGGCCCGGCTGGAGGCCTACGGCGGCAACGCCAAGCAGGCCTTCGACCCCAAGGACAACCCCTTCTACAAGAAGGGCGGCCAGCTGG |
| | | TGAAGGCCGTGCGGGTGGAGAAGACCCAGGAGTCCGGCGTGCTGCTGAACAAGAAGAACGCCTACACCATCGCCGACAACGGCGACAT |
| | | GGTGCGGGTGGACGTGTTCTGCAAGGTGGACAAGAAGGGCAAGAACCAGTACTTCATCGTGCCCATCTACGCCTGGCAGGTGGCCGAG |
| | | AACATCCTGCCCGACATCGACTGCAAGGGCTACCGGATCGACGACTCCTACACCTTCTGCTTCTCCCTGCACAAGTACGACCTGATCG |
| | | CCTTCCAGAAGGACGAGAAGTCCAAGGTGGAGTTCGCCTACTACATCAACTGCGACTCCTCCAACGGCCGGTTCTACCTGGCCTGGCA |
| | | CGACAAGGGCTCCAAGGAGCAGCAGTTCCGGATCTCCACCCAGAACCTGGTGCTGATCCAGAAGTACCAGGTGAACGAGCTGGGCAAG |
| | | GAGATCCGGCCCTGCCGGCTGAAGAAGCGGCCCCCCGTGCGGTAG |
| 805 | Exemplary ORF encoding Nme2Cas9 cleavase | ATGGCTGCTTTTAAGCCTAATCCTATTAATTATATTCTTGGTCTTGATATTGGTATTGCTTCTGTTGGTTGGGCTATGGTTGAGATTG |
| | | ATGAGGAGGAGAATCCTATTCGTCTTATTGATCTTGGTGTTCGTGTTTTTGAGCGTGCTGAGGTTCCTAAGACTGGTGATTCTCTTGC |
| | | TATGGCTCGTCGTCTTGCTCGTTCTGTTCGTCGTCTTACTCGTCGTCGTGCTCATCGTCTTCTTCGTGCTCGTCGTCTTCTTAAGCGT |
| | | GAGGGTGTTCTTCAGGCTGCTGATTTTGATGAGAATGGTCTTATTAAGTCTCTTCCTAATACTCCTTGGCAGCTTCGTGCTGCTGCTC |
| | | TTGATCGTAAGCTTACTCCTCTTGAGTGGTCTGCTGTTCTTCTTCATCTTATTAAGCATCGTGGTTATCTTTCTCAGCGTAAGAATGA |
| | | GGGTGAGACTGCTGATAAGGAGCTTGGTGCTCTTCTTAAGGGTGTTGCTAATAATGCTCATGCTCTTCAGACTGGTGATTTTCGTACT |
| | | CCTGCTGAGCTTGCTCTTAATAAGTTTGAGAAGGAGTCTGGTCATATTCGTAATCAGCGTGGTGATTATTCTCATACTTTTTCTCGTA |
| | | AGGATCTTCAGGCTGAGCTTATTCTTCTTTTTGAGAAGCAGAAGGAGTTTGGTAATCCTCATGTTTCTGGTGGTCTTAAGGAGGGTAT |
| | | TGAGACTCTTCTTATGACTCAGCGTCCTGCTCTTTCTGGTGATGCTGTTCAGAAGATGCTTGGTCATTGTACTTTTGAGCCTGCTGAG |
| | | CCTAAGGCTGCTAAGAATACTTATACTGCTGAGCGTTTTATTTGGCTTACTAAGCTTAATAATCTTCGTATTCTTGAGCAGGGTTCTG |
| | | AGCGTCCTCTTACTGATACTGAGCGTGCTACTCTTATGGATGAGCCTTATCGTAAGTCTAAGCTTACTTATGCTCAGGCTCGTAAGCT |
| | | TCTTGGTCTTGAGGATACTGCTTTTTTTAAGGGTCTTCGTTATGGTAAGGATAATGCTGAGGCTTCTACTCTTATGGAGATGAAGGCT |
| | | TATCATGCTATTTCTCGTGCTCTTGAGAAGGAGGGTCTTAAGGATAAGAAGTCTCCTCTTAATCTTTCTTCTGAGCTTCAGGATGAGA |
| | | TTGGTACTGCTTTTTCTCTTTTTAAGACTGATGAGGATATTACTGGTCGTCTTAAGGATCGTGTTCAGCCTGAGATTCTTGAGGCTCT |
| | | TCTTAAGCATATTTCTTTTGATAAGTTTGTTCAGATTTCTCTTAAGGCTCTTCGTCGTATTGTTCCTCTTATGGAGCAGGGTAAGCGT |
| | | TATGATGAGGCTTGTGCTGAGATTTATGGTGATCATTATGGTAAGAAGAATACTGAGGAGAAGATTTATCTTCCTCCTATTCCTGCTG |
| | | ATGAGATTCGTAATCCTGTTGTTCTTCGTGCTCTTTCTCAGGCTCGTAAGGTTATTAATGGTGTTGTTCGTCGTTATGGTTCTCCTGC |
| | | TCGTATTCATATTGAGACTGCTCGTGAGGTTGGTAAGTCTTTTAAGGATCGTAAGGAGATTGAGAAGCGTCAGGAGGAGAATCGTAAG |
| | | GATCGTGAGAAGGCTGCTGCTAAGTTTCGTGAGTATTTTCCTAATTTTGTTGGTGAGCCTAAGTCTAAGGATATTCTTAAGCTTCGTC |
| | | TTTATGAGCAGCAGCATGGTAAGTGTCTTTATTCTGGTAAGGAGATTAATCTTGTTCGTCTTAATGAGAAGGGTTATGTTGAGATTGA |
| | | TCATGCTCTTCCTTTTTCTCGTACTTGGGATGATTCTTTTAATAATAAGGTTCTTGTTCTTGGTTCTGAGAATCAGAATAAGGGTAAT |
| | | CAGACTCCTTATGAGTATTTTAATGGTAAGGATAATTCTCGTGAGTGGCAGGAGTTTAAGGCTCGTGTTGAGACTTCTCGTTTTCCTC |
| | | GTTCTAAGAAGCAGCGTATTCTTCTTCAGAAGTTTGATGAGGATGGTTTTAAGGAGTGTAATCTTAATGATACTCGTTATGTTAATCG |
| | | TTTTCTTTGTCAGTTTGTTGCTGATCATATTCTTCTTACTGGTAAGGGTAAGCGTCGTGTTTTTGCTTCTAATGGTCAGATTACTAAT |
| | | CTTCTTCGTGGTTTTTGGGGTCTTCGTAAGGTTCGTGCTGAGAATGATCGTCATCATGCTCTTGATGCTGTTGTTGTTGCTTGTTCTA |
| | | CTGTTGCTATGCAGCAGAAGATTACTCGTTTTGTTCGTTATAAGGAGATGAATGCTTTTGATGGTAAGACTATTGATAAGGAGACTGG |
| | | TAAGGTTCTTCATCAGAAGACTCATTTTCCTCAGCCTTGGGAGTTTTTTGCTCAGGAGGTTATGATTCGTGTTTTTGGTAAGCCTGAT |
| | | GGTAAGCCTGAGTTTGAGGAGGCTGATACTCCTGAGAAGCTTCGTACTCTTCTTGCTGAGAAGCTTTCTTCTCGTCCTGAGGCTGTTC |
| | | ATGAGTATGTTACTCCTCTTTTTGTTTCTCGTGCTCCTAATCGTAAGATGTCTGGTGCTCATAAGGATACTCTTCGTTCTGCTAAGCG |
| | | TTTTGTTAAGCATAATGAGAAGATTTCTGTTAAGCGTGTTTGGCTTACTGAGATTAAGCTTGCTGATCTTGAGAATATGGTTAATTAT |
| | | AAGAATGGTCGTGAGATTGAGCTTTATGAGGCTCTTAAGGCTCGTCTTGAGGCTTATGGTGGTAATGCTAAGCAGGCTTTTGATCCTA |
| | | AGGATAATCCTTTTTATAAGAAGGGTGGTCAGCTTGTTAAGGCTGTTCGTGTTGAGAAGACTCAGGAGTCTGGTGTTCTTCTTAATAA |
| | | GAAGAATGCTTATACTATTGCTGATAATGGTGATATGGTTCGTGTTGATGTTTTTTGTAAGGTTGATAAGAAGGGTAAGAATCAGTAT |
| | | TTTATTGTTCCTATTTATGCTTGGCAGGTTGCTGAGAATATTCTTCCTGATATTGATTGTAAGGGTTATCGTATTGATGATTCTTATA |
| | | CTTTTTGTTTTTCTCTTCATAAGTATGATCTTATTGCTTTTCAGAAGGATGAGAAGTCTAAGGTTGAGTTTGCTTATTATATTAATTG |
| | | TGATTCTTCTAATGGTCGTTTTTATCTTGCTTGGCATGATAAGGGTTCTAAGGAGCAGCAGTTTCGTATTTCTACTCAGAATCTTGTT |
| | | CTTATTCAGAAGTATCAGGTTAATGAGCTTGGTAAGGAGATTCGTCCTTGTCGTCTTAAGAAGCGTCCTCCTGTTCGTUGA |
| 806 | Exemplary ORF encoding Nme2Cas9 cleavase | ATGGCCGCCTTCAAGCCCAACCCCATCAACTACATCCTGGGCCTGGACATCGGCATCGCCTCCGTGGGCTGGGCCATGGTGGAGATCG |
| | | ACGAGGAGGAGAACCCCATCCGGCTGATCGACCTGGGCGTGCGGGTGTTCGAGCGGGCCGAGGTGCCCAAGACCGGCGACTCCCTGGC |
| | | CATGGCCCGGCGGCTGGCCCGGTCCGTGCGGCGGCTGACCCGGCGGCGGGCCCACCGGCTGCTGCGGGCCCGGCGGCTGCTGAAGCGG |
| | | GAGGGCGTGCTGCAGGCCGCCGACTTCGACGAGAACGGCCTGATCAAGTCCCTGCCCAACACCCCCTGGCAGCTGCGGGCCGCCGCCC |
| | | TGGACCGGAAGCTGACCCCCCTGGAGTGGTCCGCCGTGCTGCTGCACCTGATCAAGCACCGGGGCTACCTGTCCCAGCGGAAGAACGA |
| | | GGGCGAGACCGCCGACAAGGAGCTGGGCGCCCTGCTGAAGGGCGTGGCCAACAACGCCCACGCCCTGCAGACCGGCGACTTCCGGACC |
| | | CCCGCCGAGCTGGCCCTGAACAAGTTCGAGAAGGAGTCCGGCCACATCCGGAACCAGCGGGGCGACTACTCCCACACCTTCTCCCGGA |
| | | AGGACCTGCAGGCCGAGCTGATCCTGCTGTTCGAGAAGCAGAAGGAGTTCGGCAACCCCCACGTGTCCGGCGGCCTGAAGGAGGGCAT |
| | | CGAGACCCTGCTGATGACCCAGCGGCCCGCCCTGTCCGGCGACGCCGTGCAGAAGATGCTGGGCCACTGCACCTTCGAGCCCGCCGAG |
| | | CCCAAGGCCGCCAAGAACACCTACACCGCCGAGCGGTTCATCTGGCTGACCAAGCTGAACAACCTGCGGATCCTGGAGCAGGGCTCCG |
| | | AGCGGCCCCTGACCGACACCGAGCGGGCCACCCTGATGGACGAGCCCTACCGGAAGTCCAAGCTGACCTACGCCCAGGCCCGGAAGCT |
| | | GCTGGGCCTGGAGGACACCGCCTTCTTCAAGGGCCTGCGGTACGGCAAGGACAACGCCGAGGCCTCCACCCTGATGGAGATGAAGGCC |
| | | TACCACGCCATCTCCCGGGCCCTGGAGAAGGAGGGCCTGAAGGACAAGAAGTCCCCCCTGAACCTGTCCTCCGAGCTGCAGGACGAGA |
| | | TCGGCACCGCCTTCTCCCTGTTCAAGACCGACGAGGACATCACCGGCCGGCTGAAGGACCGGGTGCAGCCCGAGATCCTGGAGGCCCT |
| | | GCTGAAGCACATCTCCTTCGACAAGTTCGTGCAGATCTCCCTGAAGGCCCTGCGGCGGATCGTGCCCCTGATGGAGCAGGGCAAGCGG |
| | | TACGACGAGGCCTGCGCCGAGATCTACGGCGACCACTACGGCAAGAAGAACACCGAGGAGAAGATCTACCTGCCCCCCATCCCCGCCG |
| | | ACGAGATCCGGAACCCCGTGGTGCTGCGGGCCCTGTCCCAGGCCCGGAAGGTGATCAACGGCGTGGTGCGGCGGTACGGCTCCCCCGC |
| | | CCGGATCCACATCGAGACCGCCCGGGAGGTGGGCAAGTCCTTCAAGGACCGGAAGGAGATCGAGAAGCGGCAGGAGGAGAACCGGAAG |
| | | GACCGGGAGAAGGCCGCCGCCAAGTTCCGGGAGTACTTCCCCAACTTCGTGGGCGAGCCCAAGTCCAAGGACATCCTGAAGCTGCGGC |
| | | TGTACGAGCAGCAGCACGGCAAGTGCCTGTACTCCGGCAAGGAGATCAACCTGGTGCGGCTGAACGAGAAGGGCTACGTGGAGATCGA |
| | | CCACGCCCTGCCCTTCTCCCGGACCTGGGACGACTCCTTCAACAACAAGGTGCTGGTGCTGGGCTCCGAGAACCAGAACAAGGGCAAC |
| | | CAGACCCCCTACGAGTACTTCAACGGCAAGGACAACTCCCGGGAGTGGCAGGAGTTCAAGGCCCGGGTGGAGACCTCCCGGTTCCCCC |
| | | GGTCCAAGAAGCAGCGGATCCTGCTGCAGAAGTTCGACGAGGACGGCTTCAAGGAGTGCAACCTGAACGACACCCGGTACGTGAACCG |
| | | GTTCCTGTGCCAGTTCGTGGCCGACCACATCCTGCTGACCGGCAAGGGCAAGCGGCGGGTGTTCGCCTCCAACGGCCAGATCACCAAC |
| | | CTGCTGCGGGGCTTCTGGGGCCTGCGGAAGGTGCGGGCCGAGAACGACCGGCACCACGCCCTGGACGCCGTGGTGGTGGCCTGCTCCA |
| | | CCGTGGCCATGCAGCAGAAGATCACCCGGTTCGTGCGGTACAAGGAGATGAACGCCTTCGACGGCAAGACCATCGACAAGGAGACCGG |
| | | CAAGGTGCTGCACCAGAAGACCCACTTCCCCCAGCCCTGGGAGTTCTTCGCCCAGGAGGTGATGATCCGGGTGTTCGGCAAGCCCGAC |
| | | GGCAAGCCCGAGTTCGAGGAGGCCGACACCCCCGAGAAGCTGCGGACCCTGCTGGCCGAGAAGCTGTCCTCCCGGCCCGAGGCCGTGC |
| | | ACGAGTACGTGACCCCCCTGTTCGTGTCCCGGGCCCCCAACCGGAAGATGTCCGGCGCCCACAAGGACACCCTGCGGTCCGCCAAGCG |
| | | GTTCGTGAAGCACAACGAGAAGATCTCCGTGAAGCGGGTGTGGCTGACCGAGATCAAGCTGGCCGACCTGGAGAACATGGTGAACTAC |
| | | AAGAACGGCCGGGAGATCGAGCTGTACGAGGCCCTGAAGGCCCGGCTGGAGGCCTACGGCGGCAACGCCAAGCAGGCCTTCGACCCCA |
| | | AGGACAACCCCTTCTACAAGAAGGGCGGCCAGCTGGTGAAGGCCGTGCGGGTGGAGAAGACCCAGGAGTCCGGCGTGCTGCTGAACAA |
| | | GAAGAACGCCTACACCATCGCCGACAACGGCGACATGGTGCGGGTGGACGTGTTCTGCAAGGTGGACAAGAAGGGCAAGAACCAGTAC |
| | | TTCATCGTGCCCATCTACGCCTGGCAGGTGGCCGAGAACATCCTGCCCGACATCGACTGCAAGGGCTACCGGATCGACGACTCCTACA |
| | | CCTTCTGCTTCTCCCTGCACAAGTACGACCTGATCGCCTTCCAGAAGGACGAGAAGTCCAAGGTGGAGTTCGCCTACTACATCAACTG |
| | | CGACTCCTCCAACGGCCGGTTCTACCTGGCCTGGCACGACAAGGGCTCCAAGGAGCAGCAGTTCCGGATCTCCACCCAGAACCTGGTG |
| | | CTGATCCAGAAGTACCAGGTGAACGAGCTGGGCAAGGAGATCCGGCCCTGCCGGCTGAAGAAGCGGCCCCCCGTGCGGUGA |

| | | |
|---|---|---|
| 807 | Exemplary ORF encoding Nme2Cas9 cleavase | ATGGCAGCATTCAAACCAAACCCAATCAACTACATCCTAGGACTAGACATCGGAATCGCATCAGTAGGATGAGCAATGGTAGAAATCG |
| | | ACGAAGAAGAAAACCCAATCCGACTAATCGACCTAGGAGTACGAGTATTCGAACGAGCAGAAGTACCAAAAACAGGAGACTCACTAGC |
| | | AATGGCACGACGACTAGCACGATCAGTACGACGACTAACACGACGACGAGCACACCGACTACTACGAGCACGACGACTACTAAAACGA |
| | | GAAGGAGTACTACAAGCAGCAGACTTCGACGAAAACGGACTAATCAAATCACTACCAAACACACCATGACAACTACGAGCAGCAGCAC |
| | | TAGACCGAAAACTAACACCACTAGAATGATCAGCAGTACTACTACACCTAATCAAACACCGAGGATACCTATCACAACGAAAAAACGA |
| | | AGGAGAAACAGCAGACAAAGAACTAGGAGCACTACTAAAAGGAGTAGCAAACAACGCACACGCACTACAAACAGGAGACTTCCGAACA |
| | | CCAGCAGAACTAGCACTAAACAAATTCGAAAAAGAATCAGGACACATCCGAAACCAACGAGGAGACTACTCACACACATTCTCACGAA |
| | | AAGACCTACAAGCAGAACTAATCCTACTATTCGAAAAACAAAAAGAATTCGGAAACCCACACGTATCAGGAGGACTAAAAGAAGGAAT |
| | | CGAAACACTACTAATGACACAACGACCAGCACTATCAGGAGACGCAGTACAAAAAATGCTAGGACACTGCACATTCGAACCAGCAGAA |
| | | CCAAAAGCAGCAAAAAACACATACACAGCAGAACGATTCATCTGACTAACAAAACTAAACAACCTACGAATCCTAGAACAAGGATCAG |
| | | AACGACCACTAACAGACACAGAACGAGCAACACTAATGGACGAACCATACCGAAAATCAAAACTAACATACGCACAAGCACGAAAACT |
| | | ACTAGGACTAGAAGACACAGCATTCTTCAAAGGACTACGATACGGAAAAGACAACGCAGAAGCATCAACACTAATGGAAATGAAAGCA |
| | | TACCACGCAATCTCACGAGCACTAGAAAAAGAAGGACTAAAAGACAAAAAATCACCACTAAACCTATCATCAGAACTACAAGACGAAA |
| | | TCGGAACAGCATTCTCACTATTCAAAACAGACGAAGACATCACAGGACGACTAAAAGACCGAGTACAACCAGAAATCCTAGAAGCACT |
| | | ACTAAAACACATCTCATTCGACAAATTCGTACAAATCTCACTAAAAGCACTACGACGAATCGTACCACTAATGGAACAAGGAAAACGA |
| | | TACGACGAAGCATGCGCAGAAATCTACGGAGACCACTACGGAAAAAAAAACACAGAAGAAAAAATCTACCTACCACCAATCCCAGCAG |
| | | ACGAAATCCGAAACCCAGTAGTACTACGAGCACTATCACAAGCACGAAAAGTAATCAACGGAGTAGTACGACGATACGGATCACCAGC |
| | | ACGAATCCACATCGAAACAGCACGAGAAGTAGGAAAATCATTCAAAGACCGAAAAGAAATCGAAAAACGACAAGAAGAAAACCGAAAA |
| | | GACCGAGAAAAAGCAGCAGCAAAATTCCGAGAATACTTCCCAAACTTCGTAGGAGAACCAAAATCAAAAGACATCCTAAAACTACGAC |
| | | TATACGAACAACAACACGGAAAATGCCTATACTCAGGAAAAGAAATCAACCTAGTACGACTAAACGAAAAAGGATACGTAGAAATCGA |
| | | CCACGCACTACCATTCTCACGAACATGAGACGACTCATTCAACAACAAAGTACTAGTACTAGGATCAGAAAACCAAAACAAAGGAAAC |
| | | CAAACACCATACGAATACTTCAACGGAAAAGACAACTCACGAGAATGACAAGAATTCAAAGCACGAGTAGAAACATCACGATTCCCAC |
| | | GATCAAAAAAACAACGAATCCTACTACAAAAATTCGACGAAGACGGATTCAAAGAATGCAACCTAAACGACACACGATACGTAAACCG |
| | | ATTCCTATGCCAATTCGTAGCAGACCACATCCTACTAACAGGAAAAGGAAAACGACGAGTATTCGCATCAAACGGACAAATCACAAAC |
| | | CTACTACGAGGATTCTGAGGACTACGAAAAGTACGAGCAGAAAACGACCGACACCACGCACTAGACGCAGTAGTAGTAGCATGCTCAA |
| | | CAGTAGCAATGCAACAAAAAATCACACGATTCGTACGATACAAAGAAATGAACGCATTCGACGGAAAAACAATCGACAAAGAAACAGG |
| | | AAAAGTACTACACCAAAAAACACACTTCCCACAACCATGAGAATTCTTCGCACAAGAAGTAATGATCCGAGTATTCGGAAAACCAGAC |
| | | GGAAAACCAGAATTCGAAGAAGCAGACACACCAGAAAAACTACGAACACTACTAGCAGAAAAACTATCATCACGACCAGAAGCAGTAC |
| | | ACGAATACGTAACACCACTATTCGTATCACGAGCACCAAACCGAAAAATGTCAGGAGCACACAAAGACACACTACGATCAGCAAAACG |
| | | ATTCGTAAAACACAACGAAAAAATCTCAGTAAAACGAGTATGACTAACAGAAATCAAACTAGCAGACCTAGAAAACATGGTAAACTAC |
| | | AAAAACGGACGAGAAATCGAACTATACGAAGCACTAAAAGCACGACTAGAAGCATACGGAGGAAACGCAAAACAAGCATTCGACCCAA |
| | | AAGACAACCCATTCTACAAAAAAGGAGGACAACTAGTAAAAGCAGTACGAGTAGAAAAAACACAAGAATCAGGAGTACTACTAAACAA |
| | | AAAAAACGCATACACAATCGCAGACAACGGAGACATGGTACGAGTAGACGTATTCTGCAAAGTAGACAAAAAAGGAAAAAACCAATAC |
| | | TTCATCGTACCAATCTACGCATGACAAGTAGCAGAAAACATCCTACCAGACATCGACTGCAAAGGATACCGAATCGACGACTCATACA |
| | | CATTCTGCTTCTCACTACACAAATACGACCTAATCGCATTCCAAAAAGACGAAAAATCAAAAGTAGAATTCGCATACTACATCAACTG |
| | | CGACTCATCAAACGGACGATTCTACCTAGCATGACACGACAAAGGATCAAAAGAACAACAATTCCGAATCTCAACACAAAACCTAGTA |
| | | CTAATCCAAAAATACCAAGTAAACGAACTAGGAAAAGAAATCCGACCATGCCGACTAAAAAAACGACCACCAGTACGAUAA |
| 808 | Exemplary ORF for D16A Nme2Cas9 nickase | ATGGCTGCTTTTAAGCCTAATCCTATTAATTATATTCTTGGTCTTGCTATTGGTATTGCTTCTGTTGGTTGGGCTATGGTTGAGATTG |
| | | ATGAGGAGGAGAATCCTATTCGTCTTATTGATCTTGGTGTTCGTGTTTTTGAGCGTGCTGAGGTTCCTAAGACTGGTGATTCTCTTGC |
| | | TATGGCTCGTCGTCTTGCTCGTTCTGTTCGTCGTCTTACTCGTCGTCGTGCTCATCGTCTTCTTCGTGCTCGTCGTCTTCTTAAGCGT |
| | | GAGGGTGTTCTTCAGGCTGCTGATTTTGATGAGAATGGTCTTATTAAGTCTCTTCCTAATACTCCTTGGCAGCTTCGTGCTGCTGCTC |
| | | TTGATCGTAAGCTTACTCCTCTTGAGTGGTCTGCTGTTCTTCTTCATCTTATTAAGCATCGTGGTTATCTTTCTCAGCGTAAGAATGA |
| | | GGGTGAGACTGCTGATAAGGAGCTTGGTGCTCTTCTTAAGGGTGTTGCTAATAATGCTCATGCTCTTCAGACTGGTGATTTTCGTACT |
| | | CCTGCTGAGCTTGCTCTTAATAAGTTTGAGAAGGAGTCTGGTCATATTCGTAATCAGCGTGGTGATTATTCTCATACTTTTTCTCGTA |
| | | AGGATCTTCAGGCTGAGCTTATTCTTCTTTTTGAGAAGCAGAAGGAGTTTGGTAATCCTCATGTTTCTGGTGGTCTTAAGGAGGGTAT |
| | | TGAGACTCTTCTTATGACTCAGCGTCCTGCTCTTTCTGGTGATGCTGTTCAGAAGATGCTTGGTCATTGTACTTTTGAGCCTGCTGAG |
| | | CCTAAGGCTGCTAAGAATACTTATACTGCTGAGCGTTTTATTTGGCTTACTAAGCTTAATAATCTTCGTATTCTTGAGCAGGGTTCTG |
| | | AGCGTCCTCTTACTGATACTGAGCGTGCTACTCTTATGGATGAGCCTTATCGTAAGTCTAAGCTTACTTATGCTCAGGCTCGTAAGCT |
| | | TCTTGGTCTTGAGGATACTGCTTTTTTTAAGGGTCTTCGTTATGGTAAGGATAATGCTGAGGCTTCTACTCTTATGGAGATGAAGGCT |
| | | TATCATGCTATTTCTCGTGCTCTTGAGAAGGAGGGTCTTAAGGATAAGAAGTCTCCTCTTAATCTTTCTTCTGAGCTTCAGGATGAGA |
| | | TTGGTACTGCTTTTTCTCTTTTTAAGACTGATGAGGATATTACTGGTCGTCTTAAGGATCGTGTTCAGCCTGAGATTCTTGAGGCTCT |
| | | TCTTAAGCATATTTCTTTTGATAAGTTTGTTCAGATTTCTCTTAAGGCTCTTCGTCGTATTGTTCCTCTTATGGAGCAGGGTAAGCGT |
| | | TATGATGAGGCTTGTGCTGAGATTTATGGTGATCATTATGGTAAGAAGAATACTGAGGAGAAGATTTATCTTCCTCCTATTCCTGCTG |
| | | ATGAGATTCGTAATCCTGTTGTTCTTCGTGCTCTTTCTCAGGCTCGTAAGGTTATTAATGGTGTTGTTCGTCGTTATGGTTCTCCTGC |
| | | TCGTATTCATATTGAGACTGCTCGTGAGGTTGGTAAGTCTTTTAAGGATCGTAAGGAGATTGAGAAGCGTCAGGAGGAGAATCGTAAG |
| | | GATCGTGAGAAGGCTGCTGCTAAGTTTCGTGAGTATTTTCCTAATTTTGTTGGTGAGCCTAAGTCTAAGGATATTCTTAAGCTTCGTC |
| | | TTTATGAGCAGCAGCATGGTAAGTGTCTTTATTCTGGTAAGGAGATTAATCTTGTTCGTCTTAATGAGAAGGGTTATGTTGAGATTGA |
| | | TCATGCTCTTCCTTTTTCTCGTACTTGGGATGATTCTTTTAATAATAAGGTTCTTGTTCTTGGTTCTGAGAATCAGAATAAGGGTAAT |
| | | CAGACTCCTTATGAGTATTTTAATGGTAAGGATAATTCTCGTGAGTGGCAGGAGTTTAAGGCTCGTGTTGAGACTTCTCGTTTTCCTC |
| | | GTTCTAAGAAGCAGCGTATTCTTCTTCAGAAGTTTGATGAGGATGGTTTTAAGGAGTGTAATCTTAATGATACTCGTTATGTTAATCG |
| | | TTTTCTTTGTCAGTTTGTTGCTGATCATATTCTTCTTACTGGTAAGGGTAAGCGTCGTGTTTTTGCTTCTAATGGTCAGATTACTAAT |
| | | CTTCTTCGTGGTTTTTGGGGTCTTCGTAAGGTTCGTGCTGAGAATGATCGTCATCATGCTCTTGATGCTGTTGTTGTTGCTTGTTCTA |
| | | CTGTTGCTATGCAGCAGAAGATTACTCGTTTTGTTCGTTATAAGGAGATGAATGCTTTTGATGGTAAGACTATTGATAAGGAGACTGG |
| | | TAAGGTTCTTCATCAGAAGACTCATTTTCCTCAGCCTTGGGAGTTTTTTGCTCAGGAGGTTATGATTCGTGTTTTTGGTAAGCCTGAT |
| | | GGTAAGCCTGAGTTTGAGGAGGCTGATACTCCTGAGAAGCTTCGTACTCTTCTTGCTGAGAAGCTTTCTTCTCGTCCTGAGGCTGTTC |
| | | ATGAGTATGTTACTCCTCTTTTTGTTTCTCGTGCTCCTAATCGTAAGATGTCTGGTGCTCATAAGGATACTCTTCGTTCTGCTAAGCG |
| | | TTTTGTTAAGCATAATGAGAAGATTTCTGTTAAGCGTGTTTGGCTTACTGAGATTAAGCTTGCTGATCTTGAGAATATGGTTAATTAT |
| | | AAGAATGGTCGTGAGATTGAGCTTTATGAGGCTCTTAAGGCTCGTCTTGAGGCTTATGGTGGTAATGCTAAGCAGGCTTTTGATCCTA |
| | | AGGATAATCCTTTTTATAAGAAGGGTGGTCAGCTTGTTAAGGCTGTTCGTGTTGAGAAGACTCAGGAGTCTGGTGTTCTTCTTAATAA |
| | | GAAGAATGCTTATACTATTGCTGATAATGGTGATATGGTTCGTGTTGATGTTTTTTGTAAGGTTGATAAGAAGGGTAAGAATCAGTAT |
| | | TTTATTGTTCCTATTTATGCTTGGCAGGTTGCTGAGAATATTCTTCCTGATATTGATTGTAAGGGTTATCGTATTGATGATTCTTATA |
| | | CTTTTTGTTTTTCTCTTCATAAGTATGATCTTATTGCTTTTCAGAAGGATGAGAAGTCTAAGGTTGAGTTTGCTTATTATATTAATTG |
| | | TGATTCTTCTAATGGTCGTTTTTATCTTGCTTGGCATGATAAGGGTTCTAAGGAGCAGCAGTTTCGTATTTCTACTCAGAATCTTGTT |
| | | CTTATTCAGAAGTATCAGGTTAATGAGCTTGGTAAGGAGATTCGTCCTTGTCGTCTTAAGAAGCGTCCTCCTGTTCGTUGA |
| 809 | Exemplary ORF for D16A Nme2Cas9 nickase | ATGGCCGCCTTCAAGCCCAACCCCATCAACTACATCCTGGGCCTGGCCATCGGCATCGCCTCCGTGGGCTGGGCCATGGTGGAGATCG |
| | | ACGAGGAGGAGAACCCCATCCGGCTGATCGACCTGGGCGTGCGGGTGTTCGAGCGGGCCGAGGTGCCCAAGACCGGCGACTCCCTGGC |
| | | CATGGCCCGGCGGCTGGCCCGGTCCGTGCGGCGGCTGACCCGGCGGCGGGCCCACCGGCTGCTGCGGGCCCGGCGGCTGCTGAAGCGG |
| | | GAGGGCGTGCTGCAGGCCGCCGACTTCGACGAGAACGGCCTGATCAAGTCCCTGCCCAACACCCCCTGGCAGCTGCGGGCCGCCGCCC |
| | | TGGACCGGAAGCTGACCCCCCTGGAGTGGTCCGCCGTGCTGCTGCACCTGATCAAGCACCGGGGCTACCTGTCCCAGCGGAAGAACGA |
| | | GGGCGAGACCGCCGACAAGGAGCTGGGCGCCCTGCTGAAGGGCGTGGCCAACAACGCCCACGCCCTGCAGACCGGCGACTTCCGGACC |
| | | CCCGCCGAGCTGGCCCTGAACAAGTTCGAGAAGGAGTCCGGCCACATCCGGAACCAGCGGGGCGACTACTCCCACACCTTCTCCCGGA |
| | | AGGACCTGCAGGCCGAGCTGATCCTGCTGTTCGAGAAGCAGAAGGAGTTCGGCAACCCCCACGTGTCCGGCGGCCTGAAGGAGGGCAT |
| | | CGAGACCCTGCTGATGACCCAGCGGCCCGCCCTGTCCGGCGACGCCGTGCAGAAGATGCTGGGCCACTGCACCTTCGAGCCCGCCGAG |
| | | CCCAAGGCCGCCAAGAACACCTACACCGCCGAGCGGTTCATCTGGCTGACCAAGCTGAACAACCTGCGGATCCTGGAGCAGGGCTCCG |
| | | AGCGGCCCCTGACCGACACCGAGCGGGCCACCCTGATGGACGAGCCCTACCGGAAGTCCAAGCTGACCTACGCCCAGGCCCGGAAGCT |
| | | GCTGGGCCTGGAGGACACCGCCTTCTTCAAGGGCCTGCGGTACGGCAAGGACAACGCCGAGGCCTCCACCCTGATGGAGATGAAGGCC |
| | | TACCACGCCATCTCCCGGGCCCTGGAGAAGGAGGGCCTGAAGGACAAGAAGTCCCCCCTGAACCTGTCCTCCGAGCTGCAGGACGAGA |
| | | TCGGCACCGCCTTCTCCCTGTTCAAGACCGACGAGGACATCACCGGCCGGCTGAAGGACCGGGTGCAGCCCGAGATCCTGGAGGCCCT |
| | | GCTGAAGCACATCTCCTTCGACAAGTTCGTGCAGATCTCCCTGAAGGCCCTGCGGCGGATCGTGCCCCTGATGGAGCAGGGCAAGCGG |
| | | TACGACGAGGCCTGCGCCGAGATCTACGGCGACCACTACGGCAAGAAGAACACCGAGGAGAAGATCTACCTGCCCCCCATCCCCGCCG |
| | | ACGAGATCCGGAACCCCGTGGTGCTGCGGGCCCTGTCCCAGGCCCGGAAGGTGATCAACGGCGTGGTGCGGCGGTACGGCTCCCCCGC |
| | | CCGGATCCACATCGAGACCGCCCGGGAGGTGGGCAAGTCCTTCAAGGACCGGAAGGAGATCGAGAAGCGGCAGGAGGAGAACCGGAAG |
| | | GACCGGGAGAAGGCCGCCGCCAAGTTCCGGGAGTACTTCCCCAACTTCGTGGGCGAGCCCAAGTCCAAGGACATCCTGAAGCTGCGGC |
| | | TGTACGAGCAGCAGCACGGCAAGTGCCTGTACTCCGGCAAGGAGATCAACCTGGTGCGGCTGAACGAGAAGGGCTACGTGGAGATCGA |
| | | CCACGCCCTGCCCTTCTCCCGGACCTGGGACGACTCCTTCAACAACAAGGTGCTGGTGCTGGGCTCCGAGAACCAGAACAAGGGCAAC |
| | | CAGACCCCCTACGAGTACTTCAACGGCAAGGACAACTCCCGGGAGTGGCAGGAGTTCAAGGCCCGGGTGGAGACCTCCCGGTTCCCCC |
| | | GGTCCAAGAAGCAGCGGATCCTGCTGCAGAAGTTCGACGAGGACGGCTTCAAGGAGTGCAACCTGAACGACACCCGGTACGTGAACCG |
| | | GTTCCTGTGCCAGTTCGTGGCCGACCACATCCTGCTGACCGGCAAGGGCAAGCGGCGGGTGTTCGCCTCCAACGGCCAGATCACCAAC |
| | | CTGCTGCGGGGCTTCTGGGGCCTGCGGAAGGTGCGGGCCGAGAACGACCGGCACCACGCCCTGGACGCCGTGGTGGTGGCCTGCTCCA |
| | | CCGTGGCCATGCAGCAGAAGATCACCCGGTTCGTGCGGTACAAGGAGATGAACGCCTTCGACGGCAAGACCATCGACAAGGAGACCGG |
| | | CAAGGTGCTGCACCAGAAGACCCACTTCCCCCAGCCCTGGGAGTTCTTCGCCCAGGAGGTGATGATCCGGGTGTTCGGCAAGCCCGAC |
| | | GGCAAGCCCGAGTTCGAGGAGGCCGACACCCCCGAGAAGCTGCGGACCCTGCTGGCCGAGAAGCTGTCCTCCCGGCCCGAGGCCGTGC |
| | | ACGAGTACGTGACCCCCCTGTTCGTGTCCCGGGCCCCCAACCGGAAGATGTCCGGCGCCCACAAGGACACCCTGCGGTCCGCCAAGCG |
| | | GTTCGTGAAGCACAACGAGAAGATCTCCGTGAAGCGGGTGTGGCTGACCGAGATCAAGCTGGCCGACCTGGAGAACATGGTGAACTAC |
| | | AAGAACGGCCGGGAGATCGAGCTGTACGAGGCCCTGAAGGCCCGGCTGGAGGCCTACGGCGGCAACGCCAAGCAGGCCTTCGACCCCA |
| | | AGGACAACCCCTTCTACAAGAAGGGCGGCCAGCTGGTGAAGGCCGTGCGGGTGGAGAAGACCCAGGAGTCCGGCGTGCTGCTGAACAA |
| | | GAAGAACGCCTACACCATCGCCGACAACGGCGACATGGTGCGGGTGGACGTGTTCTGCAAGGTGGACAAGAAGGGCAAGAACCAGTAC |
| | | TTCATCGTGCCCATCTACGCCTGGCAGGTGGCCGAGAACATCCTGCCCGACATCGACTGCAAGGGCTACCGGATCGACGACTCCTACA |
| | | CCTTCTGCTTCTCCCTGCACAAGTACGACCTGATCGCCTTCCAGAAGGACGAGAAGTCCAAGGTGGAGTTCGCCTACTACATCAACTG |
| | | CGACTCCTCCAACGGCCGGTTCTACCTGGCCTGGCACGACAAGGGCTCCAAGGAGCAGCAGTTCCGGATCTCCACCCAGAACCTGGTG |
| | | CTGATCCAGAAGTACCAGGTGAACGAGCTGGGCAAGGAGATCCGGCCCTGCCGGCTGAAGAAGCGGCCCCCCGTGCGGUGA |
| 810 | Exemplary ORF for D16A Nme2Cas9 nickase | ATGGCCGCCTTCAAGCCCAACCCCATCAACTACATCCTGGGCCTGGCCATCGGCATCGCCTCCGTGGGCTGGGCCATGGTGGAGATCG |
| | | ACGAGGAGGAGAACCCCATCCGGCTGATCGACCTGGGCGTGCGGGTGTTCGAGCGGGCCGAGGTGCCCAAGACCGGCGACTCCCTGGC |
| | | CATGGCCCGGCGGCTGGCCCGGTCCGTGCGGCGGCTGACCCGGCGGCGGGCCCACCGGCTGCTGCGGGCCCGGCGGCTGCTGAAGCGG |
| | | GAGGGCGTGCTGCAGGCCGCCGACTTCGACGAGAACGGCCTGATCAAGTCCCTGCCCAACACCCCCTGGCAGCTGCGGGCCGCCGCCC |
| | | TGGACCGGAAGCTGACCCCCCTGGAGTGGTCCGCCGTGCTGCTGCACCTGATCAAGCACCGGGGCTACCTGTCCCAGCGGAAGAACGA |
| | | GGGCGAGACCGCCGACAAGGAGCTGGGCGCCCTGCTGAAGGGCGTGGCCAACAACGCCCACGCCCTGCAGACCGGCGACTTCCGGACC |
| | | CCCGCCGAGCTGGCCCTGAACAAGTTCGAGAAGGAGTCCGGCCACATCCGGAACCAGCGGGGCGACTACTCCCACACCTTCTCCCGGA |
| | | AGGACCTGCAGGCCGAGCTGATCCTGCTGTTCGAGAAGCAGAAGGAGTTCGGCAACCCCCACGTGTCCGGCGGCCTGAAGGAGGGCAT |
| | | CGAGACCCTGCTGATGACCCAGCGGCCCGCCCTGTCCGGCGACGCCGTGCAGAAGATGCTGGGCCACTGCACCTTCGAGCCCGCCGAG |
| | | CCCAAGGCCGCCAAGAACACCTACACCGCCGAGCGGTTCATCTGGCTGACCAAGCTGAACAACCTGCGGATCCTGGAGCAGGGCTCCG |
| | | AGCGGCCCCTGACCGACACCGAGCGGGCCACCCTGATGGACGAGCCCTACCGGAAGTCCAAGCTGACCTACGCCCAGGCCCGGAAGCT |
| | | GCTGGGCCTGGAGGACACCGCCTTCTTCAAGGGCCTGCGGTACGGCAAGGACAACGCCGAGGCCTCCACCCTGATGGAGATGAAGGCC |
| | | TACCACGCCATCTCCCGGGCCCTGGAGAAGGAGGGCCTGAAGGACAAGAAGTCCCCCCTGAACCTGTCCTCCGAGCTGCAGGACGAGA |
| | | TCGGCACCGCCTTCTCCCTGTTCAAGACCGACGAGGACATCACCGGCCGGCTGAAGGACCGGGTGCAGCCCGAGATCCTGGAGGCCCT |
| | | GCTGAAGCACATCTCCTTCGACAAGTTCGTGCAGATCTCCCTGAAGGCCCTGCGGCGGATCGTGCCCCTGATGGAGCAGGGCAAGCGG |
| | | TACGACGAGGCCTGCGCCGAGATCTACGGCGACCACTACGGCAAGAAGAACACCGAGGAGAAGATCTACCTGCCCCCCATCCCCGCCG |
| | | ACGAGATCCGGAACCCCGTGGTGCTGCGGGCCCTGTCCCAGGCCCGGAAGGTGATCAACGGCGTGGTGCGGCGGTACGGCTCCCCCGC |
| | | CCGGATCCACATCGAGACCGCCCGGGAGGTGGGCAAGTCCTTCAAGGACCGGAAGGAGATCGAGAAGCGGCAGGAGGAGAACCGGAAG |
| | | GACCGGGAGAAGGCCGCCGCCAAGTTCCGGGAGTACTTCCCCAACTTCGTGGGCGAGCCCAAGTCCAAGGACATCCTGAAGCTGCGGC |
| | | TGTACGAGCAGCAGCACGGCAAGTGCCTGTACTCCGGCAAGGAGATCAACCTGGTGCGGCTGAACGAGAAGGGCTACGTGGAGATCGA |
| | | CCACGCCCTGCCCTTCTCCCGGACCTGGGACGACTCCTTCAACAACAAGGTGCTGGTGCTGGGCTCCGAGAACCAGAACAAGGGCAAC |
| | | CAGACCCCCTACGAGTACTTCAACGGCAAGGACAACTCCCGGGAGTGGCAGGAGTTCAAGGCCCGGGTGGAGACCTCCCGGTTCCCCC |
| | | GGTCCAAGAAGCAGCGGATCCTGCTGCAGAAGTTCGACGAGGACGGCTTCAAGGAGTGCAACCTGAACGACACCCGGTACGTGAACCG |
| | | GTTCCTGTGCCAGTTCGTGGCCGACCACATCCTGCTGACCGGCAAGGGCAAGCGGCGGGTGTTCGCCTCCAACGGCCAGATCACCAAC |
| | | CTGCTGCGGGGCTTCTGGGGCCTGCGGAAGGTGCGGGCCGAGAACGACCGGCACCACGCCCTGGACGCCGTGGTGGTGGCCTGCTCCA |
| | | CCGTGGCCATGCAGCAGAAGATCACCCGGTTCGTGCGGTACAAGGAGATGAACGCCTTCGACGGCAAGACCATCGACAAGGAGACCGG |
| | | CAAGGTGCTGCACCAGAAGACCCACTTCCCCCAGCCCTGGGAGTTCTTCGCCCAGGAGGTGATGATCCGGGTGTTCGGCAAGCCCGAC |
| | | GGCAAGCCCGAGTTCGAGGAGGCCGACACCCCCGAGAAGCTGCGGACCCTGCTGGCCGAGAAGCTGTCCTCCCGGCCCGAGGCCGTGC |
| | | ACGAGTACGTGACCCCCCTGTTCGTGTCCCGGGCCCCCAACCGGAAGATGTCCGGCGCCCACAAGGACACCCTGCGGTCCGCCAAGCG |
| | | GTTCGTGAAGCACAACGAGAAGATCTCCGTGAAGCGGGTGTGGCTGACCGAGATCAAGCTGGCCGACCTGGAGAACATGGTGAACTAC |
| | | AAGAACGGCCGGGAGATCGAGCTGTACGAGGCCCTGAAGGCCCGGCTGGAGGCCTACGGCGGCAACGCCAAGCAGGCCTTCGACCCCA |
| | | AGGACAACCCCTTCTACAAGAAGGGCGGCCAGCTGGTGAAGGCCGTGCGGGTGGAGAAGACCCAGGAGTCCGGCGTGCTGCTGAACAA |
| | | GAAGAACGCCTACACCATCGCCGACAACGGCGACATGGTGCGGGTGGACGTGTTCTGCAAGGTGGACAAGAAGGGCAAGAACCAGTAC |
| | | TTCATCGTGCCCATCTACGCCTGGCAGGTGGCCGAGAACATCCTGCCCGACATCGACTGCAAGGGCTACCGGATCGACGACTCCTACA |
| | | CCTTCTGCTTCTCCCTGCACAAGTACGACCTGATCGCCTTCCAGAAGGACGAGAAGTCCAAGGTGGAGTTCGCCTACTACATCAACTG |
| | | CGACTCCTCCAACGGCCGGTTCTACCTGGCCTGGCACGACAAGGGCTCCAAGGAGCAGCAGTTCCGGATCTCCACCCAGAACCTGGTG |
| | | CTGATCCAGAAGTACCAGGTGAACGAGCTGGGCAAGGAGATCCGGCCCTGCCGGCTGAAGAAGCGGCCCCCCGTGCGGUAA |
| 811 | ORF encoding SpyCas9 base editor | ATGGAGGCCTCCCCCGCCTCCGGCCCCCGGCACCTGATGGACCCCCACATCTTCACCTCCAACTTCAACAACGGCATCGGCCGGCACA |
| | | AGACCTACCTGTGCTACGAGGTGGAGCGGCTGGACAACGGCACCTCCGTGAAGATGGACCAGCACCGGGGCTTCCTGCACAACCAGGC |
| | | CAAGAACCTGCTGTGCGGCTTCTACGGCCGGCACGCCGAGCTGCGGTTCCTGGACCTGGTGCCCTCCCTGCAGCTGGACCCCGCCCAG |
| | | ATCTACCGGGTGACCTGGTTCATCTCCTGGTCCCCCTGCTTCTCCTGGGGCTGCGCCGGCGAGGTGCGGGCCTTCCTGCAGGAGAACA |
| | | CCCACGTGCGGCTGCGGATCTTCGCCGCCCGGATCTACGACTACGACCCCCTGTACAAGGAGGCCCTGCAGATGCTGCGGGACGCCGG |
| | | CGCCCAGGTGTCCATCATGACCTACGACGAGTTCAAGCACTGCTGGGACACCTTCGTGGACCACCAGGGCTGCCCCTTCCAGCCCTGG |
| | | GACGGCCTGGACGAGCACTCCCAGGCCCTGTCCGGCCGGCTGCGGGCCATCCTGCAGAACCAGGGCAACTCCGGCTCCGAGACCCCCG |
| | | GCACCTCCGAGTCCGCCACCCCCGAGTCCGACAAGAAGTACTCCATCGGCCTGGCCATCGGCACCAACTCCGTGGGCTGGGCCGTGAT |
| | | CACCGACGAGTACAAGGTGCCCTCCAAGAAGTTCAAGGTGCTGGGCAACACCGACCGGCACTCCATCAAGAAGAACCTGATCGGCGCC |
| | | CTGCTGTTCGACTCCGGCGAGACCGCCGAGGCCACCCGGCTGAAGCGGACCGCCCGGCGGCGGTACACCCGGCGGAAGAACCGGATCT |
| | | GCTACCTGCAGGAGATCTTCTCCAACGAGATGGCCAAGGTGGACGACTCCTTCTTCCACCGGCTGGAGGAGTCCTTCCTGGTGGAGGA |
| | | GGACAAGAAGCACGAGCGGCACCCCATCTTCGGCAACATCGTGGACGAGGTGGCCTACCACGAGAAGTACCCCACCATCTACCACCTG |
| | | CGGAAGAAGCTGGTGGACTCCACCGACAAGGCCGACCTGCGGCTGATCTACCTGGCCCTGGCCCACATGATCAAGTTCCGGGGCCACT |
| | | TCCTGATCGAGGGCGACCTGAACCCCGACAACTCCGACGTGGACAAGCTGTTCATCCAGCTGGTGCAGACCTACAACCAGCTGTTCGA |
| | | GGAGAACCCCATCAACGCCTCCGGCGTGGACGCCAAGGCCATCCTGTCCGCCCGGCTGTCCAAGTCCCGGCGGCTGGAGAACCTGATC |
| | | GCCCAGCTGCCCGGCGAGAAGAAGAACGGCCTGTTCGGCAACCTGATCGCCCTGTCCCTGGGCCTGACCCCCAACTTCAAGTCCAACT |
| | | TCGACCTGGCCGAGGACGCCAAGCTGCAGCTGTCCAAGGACACCTACGACGACGACCTGGACAACCTGCTGGCCCAGATCGGCGACCA |
| | | GTACGCCGACCTGTTCCTGGCCGCCAAGAACCTGTCCGACGCCATCCTGCTGTCCGACATCCTGCGGGTGAACACCGAGATCACCAAG |
| | | GCCCCCCTGTCCGCCTCCATGATCAAGCGGTACGACGAGCACCACCAGGACCTGACCCTGCTGAAGGCCCTGGTGCGGCAGCAGCTGC |
| | | CCGAGAAGTACAAGGAGATCTTCTTCGACCAGTCCAAGAACGGCTACGCCGGCTACATCGACGGCGGCGCCTCCCAGGAGGAGTTCTA |
| | | CAAGTTCATCAAGCCCATCCTGGAGAAGATGGACGGCACCGAGGAGCTGCTGGTGAAGCTGAACCGGGAGGACCTGCTGCGGAAGCAG |
| | | CGGACCTTCGACAACGGCTCCATCCCCCACCAGATCCACCTGGGCGAGCTGCACGCCATCCTGCGGCGGCAGGAGGACTTCTACCCCT |
| | | TCCTGAAGGACAACCGGGAGAAGATCGAGAAGATCCTGACCTTCCGGATCCCCTACTACGTGGGCCCCCTGGCCCGGGGCAACTCCCG |
| | | GTTCGCCTGGATGACCCGGAAGTCCGAGGAGACCATCACCCCCTGGAACTTCGAGGAGGTGGTGGACAAGGGCGCCTCCGCCCAGTCC |
| | | TTCATCGAGCGGATGACCAACTTCGACAAGAACCTGCCCAACGAGAAGGTGCTGCCCAAGCACTCCCTGCTGTACGAGTACTTCACCG |
| | | TGTACAACGAGCTGACCAAGGTGAAGTACGTGACCGAGGGCATGCGGAAGCCCGCCTTCCTGTCCGGCGAGCAGAAGAAGGCCATCGT |
| | | GGACCTGCTGTTCAAGACCAACCGGAAGGTGACCGTGAAGCAGCTGAAGGAGGACTACTTCAAGAAGATCGAGTGCTTCGACTCCGTG |
| | | GAGATCTCCGGCGTGGAGGACCGGTTCAACGCCTCCCTGGGCACCTACCACGACCTGCTGAAGATCATCAAGGACAAGGACTTCCTGG |
| | | ACAACGAGGAGAACGAGGACATCCTGGAGGACATCGTGCTGACCCTGACCCTGTTCGAGGACCGGGAGATGATCGAGGAGCGGCTGAA |
| | | GACCTACGCCCACCTGTTCGACGACAAGGTGATGAAGCAGCTGAAGCGGCGGCGGTACACCGGCTGGGGCCGGCTGTCCCGGAAGCTG |
| | | ATCAACGGCATCCGGGACAAGCAGTCCGGCAAGACCATCCTGGACTTCCTGAAGTCCGACGGCTTCGCCAACCGGAACTTCATGCAGC |
| | | TGATCCACGACGACTCCCTGACCTTCAAGGAGGACATCCAGAAGGCCCAGGTGTCCGGCCAGGGCGACTCCCTGCACGAGCACATCGC |
| | | CAACCTGGCCGGCTCCCCCGCCATCAAGAAGGGCATCCTGCAGACCGTGAAGGTGGTGGACGAGCTGGTGAAGGTGATGGGCCGGCAC |
| | | AAGCCCGAGAACATCGTGATCGAGATGGCCCGGGAGAACCAGACCACCCAGAAGGGCCAGAAGAACTCCCGGGAGCGGATGAAGCGGA |
| | | TCGAGGAGGGCATCAAGGAGCTGGGCTCCCAGATCCTGAAGGAGCACCCCGTGGAGAACACCCAGCTGCAGAACGAGAAGCTGTACCT |
| | | GTACTACCTGCAGAACGGCCGGGACATGTACGTGGACCAGGAGCTGGACATCAACCGGCTGTCCGACTACGACGTGGACCACATCGTG |
| | | CCCCAGTCCTTCCTGAAGGACGACTCCATCGACAACAAGGTGCTGACCCGGTCCGACAAGAACCGGGGCAAGTCCGACAACGTGCCCT |
| | | CCGAGGAGGTGGTGAAGAAGATGAAGAACTACTGGCGGCAGCTGCTGAACGCCAAGCTGATCACCCAGCGGAAGTTCGACAACCTGAC |
| | | CAAGGCCGAGCGGGGCGGCCTGTCCGAGCTGGACAAGGCCGGCTTCATCAAGCGGCAGCTGGTGGAGACCCGGCAGATCACCAAGCAC |
| | | GTGGCCCAGATCCTGGACTCCCGGATGAACACCAAGTACGACGAGAACGACAAGCTGATCCGGGAGGTGAAGGTGATCACCCTGAAGT |
| | | CCAAGCTGGTGTCCGACTTCCGGAAGGACTTCCAGTTCTACAAGGTGCGGGAGATCAACAACTACCACCACGCCCACGACGCCTACCT |
| | | GAACGCCGTGGTGGGCACCGCCCTGATCAAGAAGTACCCCAAGCTGGAGTCCGAGTTCGTGTACGGCGACTACAAGGTGTACGACGTG |
| | | CGGAAGATGATCGCCAAGTCCGAGCAGGAGATCGGCAAGGCCACCGCCAAGTACTTCTTCTACTCCAACATCATGAACTTCTTCAAGA |
| | | CCGAGATCACCCTGGCCAACGGCGAGATCCGGAAGCGGCCCCTGATCGAGACCAACGGCGAGACCGGCGAGATCGTGTGGGACAAGGG |
| | | CCGGGACTTCGCCACCGTGCGGAAGGTGCTGTCCATGCCCCAGGTGAACATCGTGAAGAAGACCGAGGTGCAGACCGGCGGCTTCTCC |
| | | AAGGAGTCCATCCTGCCCAAGCGGAACTCCGACAAGCTGATCGCCCGGAAGAAGGACTGGGACCCCAAGAAGTACGGCGGCTTCGACT |
| | | CCCCCACCGTGGCCTACTCCGTGCTGGTGGTGGCCAAGGTGGAGAAGGGCAAGTCCAAGAAGCTGAAGTCCGTGAAGGAGCTGCTGGG |
| | | CATCACCATCATGGAGCGGTCCTCCTTCGAGAAGAACCCCATCGACTTCCTGGAGGCCAAGGGCTACAAGGAGGTGAAGAAGGACCTG |
| | | ATCATCAAGCTGCCCAAGTACTCCCTGTTCGAGCTGGAGAACGGCCGGAAGCGGATGCTGGCCTCCGCCGGCGAGCTGCAGAAGGGCA |
| | | ACGAGCTGGCCCTGCCCTCCAAGTACGTGAACTTCCTGTACCTGGCCTCCCACTACGAGAAGCTGAAGGGCTCCCCCGAGGACAACGA |
| | | GCAGAAGCAGCTGTTCGTGGAGCAGCACAAGCACTACCTGGACGAGATCATCGAGCAGATCTCCGAGTTCTCCAAGCGGGTGATCCTG |
| | | GCCGACGCCAACCTGGACAAGGTGCTGTCCGCCTACAACAAGCACCGGGACAAGCCCATCCGGGAGCAGGCCGAGAACATCATCCACC |
| | | TGTTCACCCTGACCAACCTGGGCGCCCCCGCCGCCTTCAAGTACTTCGACACCACCATCGACCGGAAGCGGTACACCTCCACCAAGGA |
| | | GGTGCTGGACGCCACCCTGATCCACCAGTCCATCACCGGCCTGTACGAGACCCGGATCGACCTGTCCCAGCTGGGCGGCGACGGCGGC |
| | | GGCTCCCCCAAGAAGAAGCGGAAGGTGTGA |
| 812 | ORF encoding SpyCas9 base editor with Hibit tag | ATGGAGGCCTCCCCCGCCTCCGGCCCCCGGCACCTGATGGACCCCCACATCTTCACCTCCAACTTCAACAACGGCATCGGCCGGCACA |
| | | AGACCTACCTGTGCTACGAGGTGGAGCGGCTGGACAACGGCACCTCCGTGAAGATGGACCAGCACCGGGGCTTCCTGCACAACCAGGC |
| | | CAAGAACCTGCTGTGCGGCTTCTACGGCCGGCACGCCGAGCTGCGGTTCCTGGACCTGGTGCCCTCCCTGCAGCTGGACCCCGCCCAG |
| | | ATCTACCGGGTGACCTGGTTCATCTCCTGGTCCCCCTGCTTCTCCTGGGGCTGCGCCGGCGAGGTGCGGGCCTTCCTGCAGGAGAACA |
| | | CCCACGTGCGGCTGCGGATCTTCGCCGCCCGGATCTACGACTACGACCCCCTGTACAAGGAGGCCCTGCAGATGCTGCGGGACGCCGG |
| | | CGCCCAGGTGTCCATCATGACCTACGACGAGTTCAAGCACTGCTGGGACACCTTCGTGGACCACCAGGGCTGCCCCTTCCAGCCCTGG |
| | | GACGGCCTGGACGAGCACTCCCAGGCCCTGTCCGGCCGGCTGCGGGCCATCCTGCAGAACCAGGGCAACTCCGGCTCCGAGACCCCCG |
| | | GCACCTCCGAGTCCGCCACCCCCGAGTCCGACAAGAAGTACTCCATCGGCCTGGCCATCGGCACCAACTCCGTGGGCTGGGCCGTGAT |
| | | CACCGACGAGTACAAGGTGCCCTCCAAGAAGTTCAAGGTGCTGGGCAACACCGACCGGCACTCCATCAAGAAGAACCTGATCGGCGCC |
| | | CTGCTGTTCGACTCCGGCGAGACCGCCGAGGCCACCCGGCTGAAGCGGACCGCCCGGCGGCGGTACACCCGGCGGAAGAACCGGATCT |
| | | GCTACCTGCAGGAGATCTTCTCCAACGAGATGGCCAAGGTGGACGACTCCTTCTTCCACCGGCTGGAGGAGTCCTTCCTGGTGGAGGA |
| | | GGACAAGAAGCACGAGCGGCACCCCATCTTCGGCAACATCGTGGACGAGGTGGCCTACCACGAGAAGTACCCCACCATCTACCACCTG |
| | | CGGAAGAAGCTGGTGGACTCCACCGACAAGGCCGACCTGCGGCTGATCTACCTGGCCCTGGCCCACATGATCAAGTTCCGGGGCCACT |
| | | TCCTGATCGAGGGCGACCTGAACCCCGACAACTCCGACGTGGACAAGCTGTTCATCCAGCTGGTGCAGACCTACAACCAGCTGTTCGA |
| | | GGAGAACCCCATCAACGCCTCCGGCGTGGACGCCAAGGCCATCCTGTCCGCCCGGCTGTCCAAGTCCCGGCGGCTGGAGAACCTGATC |
| | | GCCCAGCTGCCCGGCGAGAAGAAGAACGGCCTGTTCGGCAACCTGATCGCCCTGTCCCTGGGCCTGACCCCCAACTTCAAGTCCAACT |
| | | TCGACCTGGCCGAGGACGCCAAGCTGCAGCTGTCCAAGGACACCTACGACGACGACCTGGACAACCTGCTGGCCCAGATCGGCGACCA |
| | | GTACGCCGACCTGTTCCTGGCCGCCAAGAACCTGTCCGACGCCATCCTGCTGTCCGACATCCTGCGGGTGAACACCGAGATCACCAAG |
| | | GCCCCCCTGTCCGCCTCCATGATCAAGCGGTACGACGAGCACCACCAGGACCTGACCCTGCTGAAGGCCCTGGTGCGGCAGCAGCTGC |
| | | CCGAGAAGTACAAGGAGATCTTCTTCGACCAGTCCAAGAACGGCTACGCCGGCTACATCGACGGCGGCGCCTCCCAGGAGGAGTTCTA |
| | | CAAGTTCATCAAGCCCATCCTGGAGAAGATGGACGGCACCGAGGAGCTGCTGGTGAAGCTGAACCGGGAGGACCTGCTGCGGAAGCAG |
| | | CGGACCTTCGACAACGGCTCCATCCCCCACCAGATCCACCTGGGCGAGCTGCACGCCATCCTGCGGCGGCAGGAGGACTTCTACCCCT |
| | | TCCTGAAGGACAACCGGGAGAAGATCGAGAAGATCCTGACCTTCCGGATCCCCTACTACGTGGGCCCCCTGGCCCGGGGCAACTCCCG |
| | | GTTCGCCTGGATGACCCGGAAGTCCGAGGAGACCATCACCCCCTGGAACTTCGAGGAGGTGGTGGACAAGGGCGCCTCCGCCCAGTCC |
| | | TTCATCGAGCGGATGACCAACTTCGACAAGAACCTGCCCAACGAGAAGGTGCTGCCCAAGCACTCCCTGCTGTACGAGTACTTCACCG |
| | | TGTACAACGAGCTGACCAAGGTGAAGTACGTGACCGAGGGCATGCGGAAGCCCGCCTTCCTGTCCGGCGAGCAGAAGAAGGCCATCGT |
| | | GGACCTGCTGTTCAAGACCAACCGGAAGGTGACCGTGAAGCAGCTGAAGGAGGACTACTTCAAGAAGATCGAGTGCTTCGACTCCGTG |
| | | GAGATCTCCGGCGTGGAGGACCGGTTCAACGCCTCCCTGGGCACCTACCACGACCTGCTGAAGATCATCAAGGACAAGGACTTCCTGG |
| | | ACAACGAGGAGAACGAGGACATCCTGGAGGACATCGTGCTGACCCTGACCCTGTTCGAGGACCGGGAGATGATCGAGGAGCGGCTGAA |
| | | GACCTACGCCCACCTGTTCGACGACAAGGTGATGAAGCAGCTGAAGCGGCGGCGGTACACCGGCTGGGGCCGGCTGTCCCGGAAGCTG |
| | | ATCAACGGCATCCGGGACAAGCAGTCCGGCAAGACCATCCTGGACTTCCTGAAGTCCGACGGCTTCGCCAACCGGAACTTCATGCAGC |
| | | TGATCCACGACGACTCCCTGACCTTCAAGGAGGACATCCAGAAGGCCCAGGTGTCCGGCCAGGGCGACTCCCTGCACGAGCACATCGC |
| | | CAACCTGGCCGGCTCCCCCGCCATCAAGAAGGGCATCCTGCAGACCGTGAAGGTGGTGGACGAGCTGGTGAAGGTGATGGGCCGGCAC |
| | | AAGCCCGAGAACATCGTGATCGAGATGGCCCGGGAGAACCAGACCACCCAGAAGGGCCAGAAGAACTCCCGGGAGCGGATGAAGCGGA |
| | | TCGAGGAGGGCATCAAGGAGCTGGGCTCCCAGATCCTGAAGGAGCACCCCGTGGAGAACACCCAGCTGCAGAACGAGAAGCTGTACCT |
| | | GTACTACCTGCAGAACGGCCGGGACATGTACGTGGACCAGGAGCTGGACATCAACCGGCTGTCCGACTACGACGTGGACCACATCGTG |
| | | CCCCAGTCCTTCCTGAAGGACGACTCCATCGACAACAAGGTGCTGACCCGGTCCGACAAGAACCGGGGCAAGTCCGACAACGTGCCCT |
| | | CCGAGGAGGTGGTGAAGAAGATGAAGAACTACTGGCGGCAGCTGCTGAACGCCAAGCTGATCACCCAGCGGAAGTTCGACAACCTGAC |
| | | CAAGGCCGAGCGGGGCGGCCTGTCCGAGCTGGACAAGGCCGGCTTCATCAAGCGGCAGCTGGTGGAGACCCGGCAGATCACCAAGCAC |
| | | GTGGCCCAGATCCTGGACTCCCGGATGAACACCAAGTACGACGAGAACGACAAGCTGATCCGGGAGGTGAAGGTGATCACCCTGAAGT |
| | | CCAAGCTGGTGTCCGACTTCCGGAAGGACTTCCAGTTCTACAAGGTGCGGGAGATCAACAACTACCACCACGCCCACGACGCCTACCT |
| | | GAACGCCGTGGTGGGCACCGCCCTGATCAAGAAGTACCCCAAGCTGGAGTCCGAGTTCGTGTACGGCGACTACAAGGTGTACGACGTG |
| | | CGGAAGATGATCGCCAAGTCCGAGCAGGAGATCGGCAAGGCCACCGCCAAGTACTTCTTCTACTCCAACATCATGAACTTCTTCAAGA |
| | | CCGAGATCACCCTGGCCAACGGCGAGATCCGGAAGCGGCCCCTGATCGAGACCAACGGCGAGACCGGCGAGATCGTGTGGGACAAGGG |
| | | CCGGGACTTCGCCACCGTGCGGAAGGTGCTGTCCATGCCCCAGGTGAACATCGTGAAGAAGACCGAGGTGCAGACCGGCGGCTTCTCC |
| | | AAGGAGTCCATCCTGCCCAAGCGGAACTCCGACAAGCTGATCGCCCGGAAGAAGGACTGGGACCCCAAGAAGTACGGCGGCTTCGACT |
| | | CCCCCACCGTGGCCTACTCCGTGCTGGTGGTGGCCAAGGTGGAGAAGGGCAAGTCCAAGAAGCTGAAGTCCGTGAAGGAGCTGCTGGG |
| | | CATCACCATCATGGAGCGGTCCTCCTTCGAGAAGAACCCCATCGACTTCCTGGAGGCCAAGGGCTACAAGGAGGTGAAGAAGGACCTG |
| | | ATCATCAAGCTGCCCAAGTACTCCCTGTTCGAGCTGGAGAACGGCCGGAAGCGGATGCTGGCCTCCGCCGGCGAGCTGCAGAAGGGCA |
| | | ACGAGCTGGCCCTGCCCTCCAAGTACGTGAACTTCCTGTACCTGGCCTCCCACTACGAGAAGCTGAAGGGCTCCCCCGAGGACAACGA |
| | | GCAGAAGCAGCTGTTCGTGGAGCAGCACAAGCACTACCTGGACGAGATCATCGAGCAGATCTCCGAGTTCTCCAAGCGGGTGATCCTG |
| | | GCCGACGCCAACCTGGACAAGGTGCTGTCCGCCTACAACAAGCACCGGGACAAGCCCATCCGGGAGCAGGCCGAGAACATCATCCACC |
| | | TGTTCACCCTGACCAACCTGGGCGCCCCCGCCGCCTTCAAGTACTTCGACACCACCATCGACCGGAAGCGGTACACCTCCACCAAGGA |
| | | GGTGCTGGACGCCACCCTGATCCACCAGTCCATCACCGGCCTGTACGAGACCCGGATCGACCTGTCCCAGCTGGGCGGCGACGGCGGC |
| | | GGCTCCCCCAAGAAGAAGCGGAAGGTGTCCGAGTCCGCCACCCCCGAGTCCGTGTCCGGCTGGCGGCTGTTCAAGAAGATCTCCTGA |
| 813 | ORF encoding SpyCas9 | ATGGACAAGAAGTACTCCATCGGCCTGGACATCGGCACCAACTCCGTGGGCTGGGCCGTGATCACCGACGAGTACAAGGTGCCCTCCA |
| | | AGAAGTTCAAGGTGCTGGGCAACACCGACCGGCACTCCATCAAGAAGAACCTGATCGGCGCCCTGCTGTTCGACTCCGGCGAGACCGC |
| | | CGAGGCCACCCGGCTGAAGCGGACCGCCCGGCGGCGGTACACCCGGCGGAAGAACCGGATCTGCTACCTGCAGGAGATCTTCTCCAAC |
| | | GAGATGGCCAAGGTGGACGACTCCTTCTTCCACCGGCTGGAGGAGTCCTTCCTGGTGGAGGAGGACAAGAAGCACGAGCGGCACCCCA |
| | | TCTTCGGCAACATCGTGGACGAGGTGGCCTACCACGAGAAGTACCCCACCATCTACCACCTGCGGAAGAAGCTGGTGGACTCCACCGA |
| | | CAAGGCCGACCTGCGGCTGATCTACCTGGCCCTGGCCCACATGATCAAGTTCCGGGGCCACTTCCTGATCGAGGGCGACCTGAACCCC |
| | | GACAACTCCGACGTGGACAAGCTGTTCATCCAGCTGGTGCAGACCTACAACCAGCTGTTCGAGGAGAACCCCATCAACGCCTCCGGCG |
| | | TGGACGCCAAGGCCATCCTGTCCGCCCGGCTGTCCAAGTCCCGGCGGCTGGAGAACCTGATCGCCCAGCTGCCCGGCGAGAAGAAGAA |
| | | CGGCCTGTTCGGCAACCTGATCGCCCTGTCCCTGGGCCTGACCCCCAACTTCAAGTCCAACTTCGACCTGGCCGAGGACGCCAAGCTG |
| | | CAGCTGTCCAAGGACACCTACGACGACGACCTGGACAACCTGCTGGCCCAGATCGGCGACCAGTACGCCGACCTGTTCCTGGCCGCCA |
| | | AGAACCTGTCCGACGCCATCCTGCTGTCCGACATCCTGCGGGTGAACACCGAGATCACCAAGGCCCCCCTGTCCGCCTCCATGATCAA |
| | | GCGGTACGACGAGCACCACCAGGACCTGACCCTGCTGAAGGCCCTGGTGCGGCAGCAGCTGCCCGAGAAGTACAAGGAGATCTTCTTC |
| | | GACCAGTCCAAGAACGGCTACGCCGGCTACATCGACGGCGGCGCCTCCCAGGAGGAGTTCTACAAGTTCATCAAGCCCATCCTGGAGA |
| | | AGATGGACGGCACCGAGGAGCTGCTGGTGAAGCTGAACCGGGAGGACCTGCTGCGGAAGCAGCGGACCTTCGACAACGGCTCCATCCC |
| | | CCACCAGATCCACCTGGGCGAGCTGCACGCCATCCTGCGGCGGCAGGAGGACTTCTACCCCTTCCTGAAGGACAACCGGGAGAAGATC |
| | | GAGAAGATCCTGACCTTCCGGATCCCCTACTACGTGGGCCCCCTGGCCCGGGGCAACTCCCGGTTCGCCTGGATGACCCGGAAGTCCG |
| | | AGGAGACCATCACCCCCTGGAACTTCGAGGAGGTGGTGGACAAGGGCGCCTCCGCCCAGTCCTTCATCGAGCGGATGACCAACTTCGA |
| | | CAAGAACCTGCCCAACGAGAAGGTGCTGCCCAAGCACTCCCTGCTGTACGAGTACTTCACCGTGTACAACGAGCTGACCAAGGTGAAG |
| | | TACGTGACCGAGGGCATGCGGAAGCCCGCCTTCCTGTCCGGCGAGCAGAAGAAGGCCATCGTGGACCTGCTGTTCAAGACCAACCGGA |
| | | AGGTGACCGTGAAGCAGCTGAAGGAGGACTACTTCAAGAAGATCGAGTGCTTCGACTCCGTGGAGATCTCCGGCGTGGAGGACCGGTT |
| | | CAACGCCTCCCTGGGCACCTACCACGACCTGCTGAAGATCATCAAGGACAAGGACTTCCTGGACAACGAGGAGAACGAGGACATCCTG |
| | | GAGGACATCGTGCTGACCCTGACCCTGTTCGAGGACCGGGAGATGATCGAGGAGCGGCTGAAGACCTACGCCCACCTGTTCGACGACA |
| | | AGGTGATGAAGCAGCTGAAGCGGCGGCGGTACACCGGCTGGGGCCGGCTGTCCCGGAAGCTGATCAACGGCATCCGGGACAAGCAGTC |
| | | CGGCAAGACCATCCTGGACTTCCTGAAGTCCGACGGCTTCGCCAACCGGAACTTCATGCAGCTGATCCACGACGACTCCCTGACCTTC |
| | | AAGGAGGACATCCAGAAGGCCCAGGTGTCCGGCCAGGGCGACTCCCTGCACGAGCACATCGCCAACCTGGCCGGCTCCCCCGCCATCA |
| | | AGAAGGGCATCCTGCAGACCGTGAAGGTGGTGGACGAGCTGGTGAAGGTGATGGGCCGGCACAAGCCCGAGAACATCGTGATCGAGAT |
| | | GGCCCGGGAGAACCAGACCACCCAGAAGGGCCAGAAGAACTCCCGGGAGCGGATGAAGCGGATCGAGGAGGGCATCAAGGAGCTGGGC |
| | | TCCCAGATCCTGAAGGAGCACCCCGTGGAGAACACCCAGCTGCAGAACGAGAAGCTGTACCTGTACTACCTGCAGAACGGCCGGGACA |
| | | TGTACGTGGACCAGGAGCTGGACATCAACCGGCTGTCCGACTACGACGTGGACCACATCGTGCCCCAGTCCTTCCTGAAGGACGACTC |
| | | CATCGACAACAAGGTGCTGACCCGGTCCGACAAGAACCGGGGCAAGTCCGACAACGTGCCCTCCGAGGAGGTGGTGAAGAAGATGAAG |
| | | AACTACTGGCGGCAGCTGCTGAACGCCAAGCTGATCACCCAGCGGAAGTTCGACAACCTGACCAAGGCCGAGCGGGGCGGCCTGTCCG |
| | | AGCTGGACAAGGCCGGCTTCATCAAGCGGCAGCTGGTGGAGACCCGGCAGATCACCAAGCACGTGGCCCAGATCCTGGACTCCCGGAT |
| | | GAACACCAAGTACGACGAGAACGACAAGCTGATCCGGGAGGTGAAGGTGATCACCCTGAAGTCCAAGCTGGTGTCCGACTTCCGGAAG |
| | | GACTTCCAGTTCTACAAGGTGCGGGAGATCAACAACTACCACCACGCCCACGACGCCTACCTGAACGCCGTGGTGGGCACCGCCCTGA |
| | | TCAAGAAGTACCCCAAGCTGGAGTCCGAGTTCGTGTACGGCGACTACAAGGTGTACGACGTGCGGAAGATGATCGCCAAGTCCGAGCA |
| | | GGAGATCGGCAAGGCCACCGCCAAGTACTTCTTCTACTCCAACATCATGAACTTCTTCAAGACCGAGATCACCCTGGCCAACGGCGAG |
| | | ATCCGGAAGCGGCCCCTGATCGAGACCAACGGCGAGACCGGCGAGATCGTGTGGGACAAGGGCCGGGACTTCGCCACCGTGCGGAAGG |
| | | TGCTGTCCATGCCCCAGGTGAACATCGTGAAGAAGACCGAGGTGCAGACCGGCGGCTTCTCCAAGGAGTCCATCCTGCCCAAGCGGAA |
| | | CTCCGACAAGCTGATCGCCCGGAAGAAGGACTGGGACCCCAAGAAGTACGGCGGCTTCGACTCCCCCACCGTGGCCTACTCCGTGCTG |
| | | GTGGTGGCCAAGGTGGAGAAGGGCAAGTCCAAGAAGCTGAAGTCCGTGAAGGAGCTGCTGGGCATCACCATCATGGAGCGGTCCTCCT |
| | | TCGAGAAGAACCCCATCGACTTCCTGGAGGCCAAGGGCTACAAGGAGGTGAAGAAGGACCTGATCATCAAGCTGCCCAAGTACTCCCT |
| | | GTTCGAGCTGGAGAACGGCCGGAAGCGGATGCTGGCCTCCGCCGGCGAGCTGCAGAAGGGCAACGAGCTGGCCCTGCCCTCCAAGTAC |
| | | GTGAACTTCCTGTACCTGGCCTCCCACTACGAGAAGCTGAAGGGCTCCCCCGAGGACAACGAGCAGAAGCAGCTGTTCGTGGAGCAGC |
| | | ACAAGCACTACCTGGACGAGATCATCGAGCAGATCTCCGAGTTCTCCAAGCGGGTGATCCTGGCCGACGCCAACCTGGACAAGGTGCT |
| | | GTCCGCCTACAACAAGCACCGGGACAAGCCCATCCGGGAGCAGGCCGAGAACATCATCCACCTGTTCACCCTGACCAACCTGGGCGCC |
| | | CCCGCCGCCTTCAAGTACTTCGACACCACCATCGACCGGAAGCGGTACACCTCCACCAAGGAGGTGCTGGACGCCACCCTGATCCACC |
| | | AGTCCATCACCGGCCTGTACGAGACCCGGATCGACCTGTCCCAGCTGGGCGGCGACGGCGGCGGCTCCCCCAAGAAGAAGCGGAAGGT |
| | | GTGA |
| 814 | ORF encoding SpyCas9 with Hibit tag | aTggacaagaagTacTccaTcggccTggacaTcggcaccaacTccgTgggcTgggccgTgaTcaccgacgagTacaaggTgcccTcca |
| | | agaagTTcaaggTgcTgggcaacaccgaccggcacTccaTcaagaagaaccTgaTcggcgcccTgcTgTTcgacTccggcgagaccgc |
| | | cgaggccacccggcTgaagcggaccgcccggcggcggTacacccggcggaagaaccggaTcTgcTaccTgcaggagaTcTTcTccaac |
| | | gagaTggccaaggTggacgacTccTTcTTccaccggcTggaggagTccTTccTggTggaggaggacaagaagcacgagcggcacccca |
| | | TcTTcggcaacaTcgTggacgaggTggccTaccacgagaagTaccccaccaTcTaccaccTgcggaagaagcTggTggacTccaccga |
| | | caaggccgaccTgcggcTgaTcTaccTggcccTggcccacaTgaTcaagTTccggggccacTTccTgaTcgagggcgaccTgaacccc |
| | | gacaacTccgacgTggacaagcTgTTcaTccagcTggTgcagaccTacaaccagcTgTTcgaggagaaccccaTcaacgccTccggcg |
| | | TggacgccaaggccaTccTgTccgcccggcTgTccaagTcccggcggcTggagaaccTgaTcgcccagcTgcccggcgagaagaagaa |
| | | cggccTgTTcggcaaccTgaTcgcccTgTcccTgggccTgacccccaacTTcaagTccaacTTcgaccTggccgaggacgccaagcTg |
| | | cagcTgTccaaggacaccTacgacgacgaccTggacaaccTgcTggcccagaTcggcgaccagTacgccgaccTgTTccTggccgcca |
| | | agaaccTgTccgacgccaTccTgcTgTccgacaTccTgcgggTgaacaccgagaTcaccaaggccccccTgTccgccTccaTgaTcaa |
| | | gcggTacgacgagcaccaccaggaccTgacccTgcTgaaggcccTggTgcggcagcagcTgcccgagaagTacaaggagaTcTTcTTc |
| | | gaccagTccaagaacggcTacgccggcTacaTcgacggcggcgccTcccaggaggagTTcTacaagTTcaTcaagcccaTccTggaga |
| | | agaTggacggcaccgaggagcTgcTggTgaagcTgaaccgggaggaccTgcTgcggaagcagcggaccTTcgacaacggcTccaTccc |
| | | ccaccagaTccaccTgggcgagcTgcacgccaTccTgcggcggcaggaggacTTcTaccccTTccTgaaggacaaccgggagaagaTc |
| | | gagaagaTccTgaccTTccggaTccccTacTacgTgggcccccTggcccggggcaacTcccggTTcgccTggaTgacccggaagTccg |
| | | aggagaccaTcacccccTggaacTTcgaggaggTggTggacaagggcgccTccgcccagTccTTcaTcgagcggaTgaccaacTTcga |
| | | caagaaccTgcccaacgagaaggTgcTgcccaagcacTcccTgcTgTacgagTacTTcaccgTgTacaacgagcTgaccaaggTgaag |
| | | TacgTgaccgagggcaTgcggaagcccgccTTccTgTccggcgagcagaagaaggccaTcgTggaccTgcTgTTcaagaccaaccgga |
| | | aggTgaccgTgaagcagcTgaaggaggacTacTTcaagaagaTcgagTgcTTcgacTccgTggagaTcTccggcgTggaggaccggTT |
| | | caacgccTcccTgggcaccTaccacgaccTgcTgaagaTcaTcaaggacaaggacTTccTggacaacgaggagaacgaggacaTccTg |
| | | gaggacaTcgTgcTgacccTgacccTgTTcgaggaccgggagaTgaTcgaggagcggcTgaagaccTacgcccaccTgTTcgacgaca |
| | | aggTgaTgaagcagcTgaagcggcggcggTacaccggcTggggccggcTgTcccggaagcTgaTcaacggcaTccgggacaagcagTc |
| | | cggcaagaccaTccTggacTTccTgaagTccgacggcTTcgccaaccggaacTTcaTgcagcTgaTccacgacgacTcccTgaccTTc |
| | | aaggaggacaTccagaaggcccaggTgTccggccagggcgacTcccTgcacgagcacaTcgccaaccTggccggcTcccccgccaTca |
| | | agaagggcaTccTgcagaccgTgaaggTggTggacgagcTggTgaaggTgaTgggccggcacaagcccgagaacaTcgTgaTcgagaT |
| | | ggcccgggagaaccagaccacccagaagggccagaagaacTcccgggagcggaTgaagcggaTcgaggagggcaTcaaggagcTgggc |
| | | TcccagaTccTgaaggagcaccccgTggagaacacccagcTgcagaacgagaagcTgTaccTgTacTaccTgcagaacggccgggaca |
| | | TgTacgTggaccaggagcTggacaTcaaccggcTgTccgacTacgacgTggaccacaTcgTgccccagTccTTccTgaaggacgacTc |
| | | caTcgacaacaaggTgcTgacccggTccgacaagaaccggggcaagTccgacaacgTgcccTccgaggaggTggTgaagaagaTgaag |
| | | aacTacTggcggcagcTgcTgaacgccaagcTgaTcacccagcggaagTTcgacaaccTgaccaaggccgagcggggcggccTgTccg |
| | | agcTggacaaggccggcTTcaTcaagcggcagcTggTggagacccggcagaTcaccaagcacgTggcccagaTccTggacTcccggaT |
| | | gaacaccaagTacgacgagaacgacaagcTgaTccgggaggTgaaggTgaTcacccTgaagTccaagcTggTgTccgacTTccggaag |
| | | gacTTccagTTcTacaaggTgcgggagaTcaacaacTaccaccacgcccacgacgccTaccTgaacgccgTggTgggcaccgcccTga |
| | | TcaagaagTaccccaagcTggagTccgagTTcgTgTacggcgacTacaaggTgTacgacgTgcggaagaTgaTcgccaagTccgagca |
| | | ggagaTcggcaaggccaccgccaagTacTTcTTcTacTccaacaTcaTgaacTTcTTcaagaccgagaTcacccTggccaacggcgag |
| | | aTccggaagcggccccTgaTcgagaccaacggcgagaccggcgagaTcgTgTgggacaagggccgggacTTcgccaccgTgcggaagg |
| | | TgcTgTccaTgccccaggTgaacaTcgTgaagaagaccgaggTgcagaccggcggcTTcTccaaggagTccaTccTgcccaagcggaa |
| | | cTccgacaagcTgaTcgcccggaagaaggacTgggaccccaagaagTacggcggcTTcgacTcccccaccgTggccTacTccgTgcTg |
| | | gTggTggccaaggTggagaagggcaagTccaagaagcTgaagTccgTgaaggagcTgcTgggcaTcaccaTcaTggagcggTccTccT |
| | | TcgagaagaaccccaTcgacTTccTggaggccaagggcTacaaggaggTgaagaaggaccTgaTcaTcaagcTgcccaagTacTcccT |
| | | gTTcgagcTggagaacggccggaagcggaTgcTggccTccgccggcgagcTgcagaagggcaacgagcTggcccTgcccTccaagTac |
| | | gTgaacTTccTgTaccTggccTcccacTacgagaagcTgaagggcTcccccgaggacaacgagcagaagcagcTgTTcgTggagcagc |
| | | acaagcacTaccTggacgagaTcaTcgagcagaTcTccgagTTcTccaagcgggTgaTccTggccgacgccaaccTggacaaggTgcT |
| | | gTccgccTacaacaagcaccgggacaagcccaTccgggagcaggccgagaacaTcaTccaccTgTTcacccTgaccaaccTgggcgcc |
| | | cccgccgccTTcaagTacTTcgacaccaccaTcgaccggaagcggTacaccTccaccaaggaggTgcTggacgccacccTgaTccacc |
| | | agTccaTcaccggccTgTacgagacccggaTcgaccTgTcccagcTgggcggcgacggcggcggcTcccccaagaagaagcggaaggT |
| | | gTccgagTccgccacccccgagTccgTgTccggcTggcggcTgTTcaagaagaTcTccTga |
| 815 | ORF encoding SpyCas9 base editor with | ATGGAGGCCTCCCCCGCCTCCGGCCCCCGGCACCTGATGGACCCCCACAT |
| | Hibit tag | CTTCACCTCCAACTTCAACAACGGCATCGGCCGGCACAAGACCTACCTGT |
| | | GCTACGAGGTGGAGCGGCTGGACAACGGCACCTCCGTGAAGATGGACCAG |
| | | CACCGGGGCTTCCTGCACAACCAGGCCAAGAACCTGCTGTGCGGCTTCTA |
| | | CGGCCGGCACGCCGAGCTGCGGTTCCTGGACCTGGTGCCCTCCCTGCAGC |
| | | TGGACCCCGCCCAGATCTACCGGGTGACCTGGTTCATCTCCTGGTCCCCC |
| | | TGCTTCTCCTGGGGCTGCGCCGGCGAGGTGCGGGCCTTCCTGCAGGAGAA |
| | | CACCCACGTGCGGCTGCGGATCTTCGCCGCCCGGATCTACGACTACGACC |
| | | CCCTGTACAAGGAGGCCCTGCAGATGCTGCGGGACGCCGGCGCCCAGGTG |
| | | TCCATCATGACCTACGACGAGTTCAAGCACTGCTGGGACACCTTCGTGGA |
| | | CCACCAGGGCTGCCCCTTCCAGCCCTGGGACGGCCTGGACGAGCACTCCC |
| | | AGGCCCTGTCCGGCCGGCTGCGGGCCATCCTGCAGAACCAGGGCAACTCC |
| | | GGCTCCGAGACCCCCGGCACCTCCGAGTCCGCCACCCCCGAGTCCGACAA |
| | | GAAGTACTCCATCGGCCTGGCCATCGGCACCAACTCCGTGGGCTGGGCCG |
| | | TGATCACCGACGAGTACAAGGTGCCCTCCAAGAAGTTCAAGGTGCTGGGC |
| | | AACACCGACCGGCACTCCATCAAGAAGAACCTGATCGGCGCCCTGCTGTT |
| | | CGACTCCGGCGAGACCGCCGAGGCCACCCGGCTGAAGCGGACCGCCCGGC |
| | | GGCGGTACACCCGGCGGAAGAACCGGATCTGCTACCTGCAGGAGATCTTC |
| | | TCCAACGAGATGGCCAAGGTGGACGACTCCTTCTTCCACCGGCTGGAGGA |
| | | GTCCTTCCTGGTGGAGGAGGACAAGAAGCACGAGCGGCACCCCATCTTCG |
| | | GCAACATCGTGGACGAGGTGGCCTACCACGAGAAGTACCCCACCATCTAC |
| | | CACCTGCGGAAGAAGCTGGTGGACTCCACCGACAAGGCCGACCTGCGGCT |
| | | GATCTACCTGGCCCTGGCCCACATGATCAAGTTCCGGGGCCACTTCCTGA |
| | | TCGAGGGCGACCTGAACCCCGACAACTCCGACGTGGACAAGCTGTTCATC |
| | | CAGCTGGTGCAGACCTACAACCAGCTGTTCGAGGAGAACCCCATCAACGC |
| | | CTCCGGCGTGGACGCCAAGGCCATCCTGTCCGCCCGGCTGTCCAAGTCCC |
| | | GGCGGCTGGAGAACCTGATCGCCCAGCTGCCCGGCGAGAAGAAGAACGGC |
| | | CTGTTCGGCAACCTGATCGCCCTGTCCCTGGGCCTGACCCCCAACTTCAA |
| | | GTCCAACTTCGACCTGGCCGAGGACGCCAAGCTGCAGCTGTCCAAGGACA |
| | | CCTACGACGACGACCTGGACAACCTGCTGGCCCAGATCGGCGACCAGTAC |
| | | GCCGACCTGTTCCTGGCCGCCAAGAACCTGTCCGACGCCATCCTGCTGTC |
| | | CGACATCCTGCGGGTGAACACCGAGATCACCAAGGCCCCCCTGTCCGCCT |
| | | CCATGATCAAGCGGTACGACGAGCACCACCAGGACCTGACCCTGCTGAAG |
| | | GCCCTGGTGCGGCAGCAGCTGCCCGAGAAGTACAAGGAGATCTTCTTCGA |
| | | CCAGTCCAAGAACGGCTACGCCGGCTACATCGACGGCGGCGCCTCCCAGG |
| | | AGGAGTTCTACAAGTTCATCAAGCCCATCCTGGAGAAGATGGACGGCACC |
| | | GAGGAGCTGCTGGTGAAGCTGAACCGGGAGGACCTGCTGCGGAAGCAGCG |
| | | GACCTTCGACAACGGCTCCATCCCCCACCAGATCCACCTGGGCGAGCTGC |
| | | ACGCCATCCTGCGGCGGCAGGAGGACTTCTACCCCTTCCTGAAGGACAAC |
| | | CGGGAGAAGATCGAGAAGATCCTGACCTTCCGGATCCCCTACTACGTGGG |
| | | CCCCCTGGCCCGGGGCAACTCCCGGTTCGCCTGGATGACCCGGAAGTCCG |
| | | AGGAGACCATCACCCCCTGGAACTTCGAGGAGGTGGTGGACAAGGGCGCC |
| | | TCCGCCCAGTCCTTCATCGAGCGGATGACCAACTTCGACAAGAACCTGCC |
| | | CAACGAGAAGGTGCTGCCCAAGCACTCCCTGCTGTACGAGTACTTCACCG |
| | | TGTACAACGAGCTGACCAAGGTGAAGTACGTGACCGAGGGCATGCGGAAG |
| | | CCCGCCTTCCTGTCCGGCGAGCAGAAGAAGGCCATCGTGGACCTGCTGTT |
| | | CAAGACCAACCGGAAGGTGACCGTGAAGCAGCTGAAGGAGGACTACTTCA |
| | | AGAAGATCGAGTGCTTCGACTCCGTGGAGATCTCCGGCGTGGAGGACCGG |
| | | TTCAACGCCTCCCTGGGCACCTACCACGACCTGCTGAAGATCATCAAGGA |
| | | CAAGGACTTCCTGGACAACGAGGAGAACGAGGACATCCTGGAGGACATCG |
| | | TGCTGACCCTGACCCTGTTCGAGGACCGGGAGATGATCGAGGAGCGGCTG |
| | | AAGACCTACGCCCACCTGTTCGACGACAAGGTGATGAAGCAGCTGAAGCG |
| | | GCGGCGGTACACCGGCTGGGGCCGGCTGTCCCGGAAGCTGATCAACGGCA |
| | | TCCGGGACAAGCAGTCCGGCAAGACCATCCTGGACTTCCTGAAGTCCGAC |
| | | GGCTTCGCCAACCGGAACTTCATGCAGCTGATCCACGACGACTCCCTGAC |
| | | CTTCAAGGAGGACATCCAGAAGGCCCAGGTGTCCGGCCAGGGCGACTCCC |
| | | TGCACGAGCACATCGCCAACCTGGCCGGCTCCCCCGCCATCAAGAAGGGC |
| | | ATCCTGCAGACCGTGAAGGTGGTGGACGAGCTGGTGAAGGTGATGGGCCG |
| | | GCACAAGCCCGAGAACATCGTGATCGAGATGGCCCGGGAGAACCAGACCA |
| | | CCCAGAAGGGCCAGAAGAACTCCCGGGAGCGGATGAAGCGGATCGAGGAG |
| | | GGCATCAAGGAGCTGGGCTCCCAGATCCTGAAGGAGCACCCCGTGGAGAA |
| | | CACCCAGCTGCAGAACGAGAAGCTGTACCTGTACTACCTGCAGAACGGCC |
| | | GGGACATGTACGTGGACCAGGAGCTGGACATCAACCGGCTGTCCGACTAC |
| | | GACGTGGACCACATCGTGCCCCAGTCCTTCCTGAAGGACGACTCCATCGA |
| | | CAACAAGGTGCTGACCCGGTCCGACAAGAACCGGGGCAAGTCCGACAACG |
| | | TGCCCTCCGAGGAGGTGGTGAAGAAGATGAAGAACTACTGGCGGCAGCTG |
| | | CTGAACGCCAAGCTGATCACCCAGCGGAAGTTCGACAACCTGACCAAGGC |
| | | CGAGCGGGGCGGCCTGTCCGAGCTGGACAAGGCCGGCTTCATCAAGCGGC |
| | | AGCTGGTGGAGACCCGGCAGATCACCAAGCACGTGGCCCAGATCCTGGAC |
| | | TCCCGGATGAACACCAAGTACGACGAGAACGACAAGCTGATCCGGGAGGT |
| | | GAAGGTGATCACCCTGAAGTCCAAGCTGGTGTCCGACTTCCGGAAGGACT |
| | | TCCAGTTCTACAAGGTGCGGGAGATCAACAACTACCACCACGCCCACGAC |
| | | GCCTACCTGAACGCCGTGGTGGGCACCGCCCTGATCAAGAAGTACCCCAA |
| | | GCTGGAGTCCGAGTTCGTGTACGGCGACTACAAGGTGTACGACGTGCGGA |
| | | AGATGATCGCCAAGTCCGAGCAGGAGATCGGCAAGGCCACCGCCAAGTAC |
| | | TTCTTCTACTCCAACATCATGAACTTCTTCAAGACCGAGATCACCCTGGC |
| | | CAACGGCGAGATCCGGAAGCGGCCCCTGATCGAGACCAACGGCGAGACCG |
| | | GCGAGATCGTGTGGGACAAGGGCCGGGACTTCGCCACCGTGCGGAAGGTG |
| | | CTGTCCATGCCCCAGGTGAACATCGTGAAGAAGACCGAGGTGCAGACCGG |
| | | CGGCTTCTCCAAGGAGTCCATCCTGCCCAAGCGGAACTCCGACAAGCTGA |
| | | TCGCCCGGAAGAAGGACTGGGACCCCAAGAAGTACGGCGGCTTCGACTCC |
| | | CCCACCGTGGCCTACTCCGTGCTGGTGGTGGCCAAGGTGGAGAAGGGCAA |
| | | GTCCAAGAAGCTGAAGTCCGTGAAGGAGCTGCTGGGCATCACCATCATGG |
| | | AGCGGTCCTCCTTCGAGAAGAACCCCATCGACTTCCTGGAGGCCAAGGGC |
| | | TACAAGGAGGTGAAGAAGGACCTGATCATCAAGCTGCCCAAGTACTCCCT |
| | | GTTCGAGCTGGAGAACGGCCGGAAGCGGATGCTGGCCTCCGCCGGCGAGC |
| | | TGCAGAAGGGCAACGAGCTGGCCCTGCCCTCCAAGTACGTGAACTTCCTG |
| | | TACCTGGCCTCCCACTACGAGAAGCTGAAGGGCTCCCCCGAGGACAACGA |
| | | GCAGAAGCAGCTGTTCGTGGAGCAGCACAAGCACTACCTGGACGAGATCA |
| | | TCGAGCAGATCTCCGAGTTCTCCAAGCGGGTGATCCTGGCCGACGCCAAC |
| | | CTGGACAAGGTGCTGTCCGCCTACAACAAGCACCGGGACAAGCCCATCCG |
| | | GGAGCAGGCCGAGAACATCATCCACCTGTTCACCCTGACCAACCTGGGCG |
| | | CCCCCGCCGCCTTCAAGTACTTCGACACCACCATCGACCGGAAGCGGTAC |
| | | ACCTCCACCAAGGAGGTGCTGGACGCCACCCTGATCCACCAGTCCATCAC |
| | | CGGCCTGTACGAGACCCGGATCGACCTGTCCCAGCTGGGCGGCGACTCCG |
| | | GCGGCTCCGGCGGCTCCGGCGGCTCCACCAACCTGTCCGACATCATCGAG |
| | | AAGGAGACCGGCAAGCAGCTGGTGATCCAGGAGTCCATCCTGATGCTGCC |
| | | CGAGGAGGTGGAGGAGGTGATCGGCAACAAGCCCGAGTCCGACATCCTGG |
| | | TGCACACCGCCTACGACGAGTCCACCGACGAGAACGTGATGCTGCTGACC |
| | | TCCGACGCCCCCGAGTACAAGCCCTGGGCCCTGGTGATCCAGGACTCCAA |
| | | CGGCGAGAACAAGATCAAGATGCTGTCCGGCGGCTCCGGCGGCTCCGGCG |
| | | GCTCCACCAACCTGTCCGACATCATCGAGAAGGAGACCGGCAAGCAGCTG |
| | | GTGATCCAGGAGTCCATCCTGATGCTGCCCGAGGAGGTGGAGGAGGTGAT |
| | | CGGCAACAAGCCCGAGTCCGACATCCTGGTGCACACCGCCTACGACGAGT |
| | | CCACCGACGAGAACGTGATGCTGCTGACCTCCGACGCCCCCGAGTACAAG |
| | | CCCTGGGCCCTGGTGATCCAGGACTCCAACGGCGAGAACAAGATCAAGAT |
| | | GCTGTCCGGCGGCTCCAAGCGGACCGCCGACGGCTCCGAGTTCGAGCCCA |
| | | AGAAGAAGCGGAAGGTGTCCGAGTCCGCCACCCCCGAGTCCGTGTCCGGC |
| | | TGGCGGCTGTTCAAGAAGATCTCCTGA |
| 816 | ORF encoding SpyCas9 | ATGGACAAGAAGTACAGCATCGGACTGGACATCGGAACAAACAGCGTCGGATGGGCAGTCATCACAGACGAATACAAGGTCCCGAGCA |
| | | AGAAGTTCAAGGTCCTGGGAAACACAGACAGACACAGCATCAAGAAGAACCTGATCGGAGCACTGCTGTTCGACAGCGGAGAAACAGC |
| | | AGAAGCAACAAGACTGAAGAGAACAGCAAGAAGAAGATACACAAGAAGAAAGAACAGAATCTGCTACCTGCAGGAAATCTTCAGCAAC |
| | | GAAATGGCAAAGGTCGACGACAGCTTCTTCCACAGACTGGAAGAAAGCTTCCTGGTCGAAGAAGACAAGAAGCACGAAAGACACCCGA |
| | | TCTTCGGAAACATCGTCGACGAAGTCGCATACCACGAAAAGTACCCGACAATCTACCACCTGAGAAAGAAGCTGGTCGACAGCACAGA |
| | | CAAGGCAGACCTGAGACTGATCTACCTGGCACTGGCACACATGATCAAGTTCAGAGGACACTTCCTGATCGAAGGAGACCTGAACCCG |
| | | GACAACAGCGACGTCGACAAGCTGTTCATCCAGCTGGTCCAGACATACAACCAGCTGTTCGAAGAAAACCCGATCAACGCAAGCGGAG |
| | | TCGACGCAAAGGCAATCCTGAGCGCAAGACTGAGCAAGAGCAGAAGACTGGAAAACCTGATCGCACAGCTGCCGGGAGAAAAGAAGAA |
| | | CGGACTGTTCGGAAACCTGATCGCACTGAGCCTGGGACTGACACCGAACTTCAAGAGCAACTTCGACCTGGCAGAAGACGCAAAGCTG |
| | | CAGCTGAGCAAGGACACATACGACGACGACCTGGACAACCTGCTGGCACAGATCGGAGACCAGTACGCAGACCTGTTCCTGGCAGCAA |
| | | AGAACCTGAGCGACGCAATCCTGCTGAGCGACATCCTGAGAGTCAACACAGAAATCACAAAGGCACCGCTGAGCGCAAGCATGATCAA |
| | | GAGATACGACGAACACCACCAGGACCTGACACTGCTGAAGGCACTGGTCAGACAGCAGCTGCCGGAAAAGTACAAGGAAATCTTCTTC |
| | | GACCAGAGCAAGAACGGATACGCAGGATACATCGACGGAGGAGCAAGCCAGGAAGAATTCTACAAGTTCATCAAGCCGATCCTGGAAA |
| | | AGATGGACGGAACAGAAGAACTGCTGGTCAAGCTGAACAGAGAAGACCTGCTGAGAAAGCAGAGAACATTCGACAACGGAAGCATCCC |
| | | GCACCAGATCCACCTGGGAGAACTGCACGCAATCCTGAGAAGACAGGAAGACTTCTACCCGTTCCTGAAGGACAACAGAGAAAAGATC |
| | | GAAAAGATCCTGACATTCAGAATCCCGTACTACGTCGGACCGCTGGCAAGAGGAAACAGCAGATTCGCATGGATGACAAGAAAGAGCG |
| | | AAGAAACAATCACACCGTGGAACTTCGAAGAAGTCGTCGACAAGGGAGCAAGCGCACAGAGCTTCATCGAAAGAATGACAAACTTCGA |
| | | CAAGAACCTGCCGAACGAAAAGGTCCTGCCGAAGCACAGCCTGCTGTACGAATACTTCACAGTCTACAACGAACTGACAAAGGTCAAG |
| | | TACGTCACAGAAGGAATGAGAAAGCCGGCATTCCTGAGCGGAGAACAGAAGAAGGCAATCGTCGACCTGCTGTTCAAGACAAACAGAA |
| | | AGGTCACAGTCAAGCAGCTGAAGGAAGACTACTTCAAGAAGATCGAATGCTTCGACAGCGTCGAAATCAGCGGAGTCGAAGACAGATT |
| | | CAACGCAAGCCTGGGAACATACCACGACCTGCTGAAGATCATCAAGGACAAGGACTTCCTGGACAACGAAGAAAACGAAGACATCCTG |
| | | GAAGACATCGTCCTGACACTGACACTGTTCGAAGACAGAGAAATGATCGAAGAAAGACTGAAGACATACGCACACCTGTTCGACGACA |
| | | AGGTCATGAAGCAGCTGAAGAGAAGAAGATACACAGGATGGGGAAGACTGAGCAGAAAGCTGATCAACGGAATCAGAGACAAGCAGAG |
| | | CGGAAAGACAATCCTGGACTTCCTGAAGAGCGACGGATTCGCAAACAGAAACTTCATGCAGCTGATCCACGACGACAGCCTGACATTC |
| | | AAGGAAGACATCCAGAAGGCACAGGTCAGCGGACAGGGAGACAGCCTGCACGAACACATCGCAAACCTGGCAGGAAGCCCGGCAATCA |
| | | AGAAGGGAATCCTGCAGACAGTCAAGGTCGTCGACGAACTGGTCAAGGTCATGGGAAGACACAAGCCGGAAAACATCGTCATCGAAAT |
| | | GGCAAGAGAAAACCAGACAACACAGAAGGGACAGAAGAACAGCAGAGAAAGAATGAAGAGAATCGAAGAAGGAATCAAGGAACTGGGA |
| | | AGCCAGATCCTGAAGGAACACCCGGTCGAAAACACACAGCTGCAGAACGAAAAGCTGTACCTGTACTACCTGCAGAACGGAAGAGACA |
| | | TGTACGTCGACCAGGAACTGGACATCAACAGACTGAGCGACTACGACGTCGACCACATCGTCCCGCAGAGCTTCCTGAAGGACGACAG |
| | | CATCGACAACAAGGTCCTGACAAGAAGCGACAAGAACAGAGGAAAGAGCGACAACGTCCCGAGCGAAGAAGTCGTCAAGAAGATGAAG |
| | | AACTACTGGAGACAGCTGCTGAACGCAAAGCTGATCACACAGAGAAAGTTCGACAACCTGACAAAGGCAGAGAGAGGAGGACTGAGCG |
| | | AACTGGACAAGGCAGGATTCATCAAGAGACAGCTGGTCGAAACAAGACAGATCACAAAGCACGTCGCACAGATCCTGGACAGCAGAAT |
| | | GAACACAAAGTACGACGAAAACGACAAGCTGATCAGAGAAGTCAAGGTCATCACACTGAAGAGCAAGCTGGTCAGCGACTTCAGAAAG |
| | | GACTTCCAGTTCTACAAGGTCAGAGAAATCAACAACTACCACCACGCACACGACGCATACCTGAACGCAGTCGTCGGAACAGCACTGA |
| | | TCAAGAAGTACCCGAAGCTGGAAAGCGAATTCGTCTACGGAGACTACAAGGTCTACGACGTCAGAAAGATGATCGCAAAGAGCGAACA |
| | | GGAAATCGGAAAGGCAACAGCAAAGTACTTCTTCTACAGCAACATCATGAACTTCTTCAAGACAGAAATCACACTGGCAAACGGAGAA |
| | | ATCAGAAAGAGACCGCTGATCGAAACAAACGGAGAAACAGGAGAAATCGTCTGGGACAAGGGAAGAGACTTCGCAACAGTCAGAAAGG |
| | | TCCTGAGCATGCCGCAGGTCAACATCGTCAAGAAGACAGAAGTCCAGACAGGAGGATTCAGCAAGGAAAGCATCCTGCCGAAGAGAAA |
| | | CAGCGACAAGCTGATCGCAAGAAAGAAGGACTGGGACCCGAAGAAGTACGGAGGATTCGACAGCCCGACAGTCGCATACAGCGTCCTG |
| | | GTCGTCGCAAAGGTCGAAAAGGGAAAGAGCAAGAAGCTGAAGAGCGTCAAGGAACTGCTGGGAATCACAATCATGGAAAGAAGCAGCT |
| | | TCGAAAAGAACCCGATCGACTTCCTGGAAGCAAAGGGATACAAGGAAGTCAAGAAGGACCTGATCATCAAGCTGCCGAAGTACAGCCT |
| | | GTTCGAACTGGAAAACGGAAGAAAGAGAATGCTGGCAAGCGCAGGAGAACTGCAGAAGGGAAACGAACTGGCACTGCCGAGCAAGTAC |
| | | GTCAACTTCCTGTACCTGGCAAGCCACTACGAAAAGCTGAAGGGAAGCCCGGAAGACAACGAACAGAAGCAGCTGTTCGTCGAACAGC |
| | | ACAAGCACTACCTGGACGAAATCATCGAACAGATCAGCGAATTCAGCAAGAGAGTCATCCTGGCAGACGCAAACCTGGACAAGGTCCT |
| | | GAGCGCATACAACAAGCACAGAGACAAGCCGATCAGAGAACAGGCAGAAAACATCATCCACCTGTTCACACTGACAAACCTGGGAGCA |
| | | CCGGCAGCATTCAAGTACTTCGACACAACAATCGACAGAAAGAGATACACAAGCACAAAGGAAGTCCTGGACGCAACACTGATCCACC |
| | | AGAGCATCACAGGACTGTACGAAACAAGAATCGACCTGAGCCAGCTGGGAGGAGACGGAGGAGGAAGCCCGAAGAAGAAGAGAAAGGT |
| | | CTAG |
| 817 | ORF encoding SpyCas9 | ATGGACAAGAAGTACAGCATCGGCCTCGACATCGGCACCAACAGCGTCGGCTGGGCCGTCATCACCGACGAGTACAAGGTCCCCAGCA |
| | | AGAAGTTCAAGGTCCTCGGCAACACCGACCGCCACAGCATCAAGAAGAACCTCATCGGCGCCCTCCTCTTCGACAGCGGCGAGACCGC |
| | | CGAGGCCACCCGCCTCAAGCGCACCGCCCGCCGCCGCTACACCCGCCGCAAGAACCGCATCTGCTACCTCCAGGAGATCTTCAGCAAC |
| | | GAGATGGCCAAGGTCGACGACAGCTTCTTCCACCGCCTCGAGGAGAGCTTCCTCGTCGAGGAGGACAAGAAGCACGAGCGCCACCCCA |
| | | TCTTCGGCAACATCGTCGACGAGGTCGCCTACCACGAGAAGTACCCCACCATCTACCACCTCCGCAAGAAGCTCGTCGACAGCACCGA |
| | | CAAGGCCGACCTCCGCCTCATCTACCTCGCCCTCGCCCACATGATCAAGTTCCGCGGCCACTTCCTCATCGAGGGCGACCTCAACCCC |
| | | GACAACAGCGACGTCGACAAGCTCTTCATCCAGCTCGTCCAGACCTACAACCAGCTCTTCGAGGAGAACCCCATCAACGCCAGCGGCG |
| | | TCGACGCCAAGGCCATCCTCAGCGCCCGCCTCAGCAAGAGCCGCCGCCTCGAGAACCTCATCGCCCAGCTCCCCGGCGAGAAGAAGAA |
| | | CGGCCTCTTCGGCAACCTCATCGCCCTCAGCCTCGGCCTCACCCCCAACTTCAAGAGCAACTTCGACCTCGCCGAGGACGCCAAGCTC |
| | | CAGCTCAGCAAGGACACCTACGACGACGACCTCGACAACCTCCTCGCCCAGATCGGCGACCAGTACGCCGACCTCTTCCTCGCCGCCA |
| | | AGAACCTCAGCGACGCCATCCTCCTCAGCGACATCCTCCGCGTCAACACCGAGATCACCAAGGCCCCCCTCAGCGCCAGCATGATCAA |
| | | GCGCTACGACGAGCACCACCAGGACCTCACCCTCCTCAAGGCCCTCGTCCGCCAGCAGCTCCCCGAGAAGTACAAGGAGATCTTCTTC |
| | | GACCAGAGCAAGAACGGCTACGCCGGCTACATCGACGGCGGCGCCAGCCAGGAGGAGTTCTACAAGTTCATCAAGCCCATCCTCGAGA |
| | | AGATGGACGGCACCGAGGAGCTCCTCGTCAAGCTCAACCGCGAGGACCTCCTCCGCAAGCAGCGCACCTTCGACAACGGCAGCATCCC |
| | | CCACCAGATCCACCTCGGCGAGCTCCACGCCATCCTCCGCCGCCAGGAGGACTTCTACCCCTTCCTCAAGGACAACCGCGAGAAGATC |
| | | GAGAAGATCCTCACCTTCCGCATCCCCTACTACGTCGGCCCCCTCGCCCGCGGCAACAGCCGCTTCGCCTGGATGACCCGCAAGAGCG |
| | | AGGAGACCATCACCCCCTGGAACTTCGAGGAGGTCGTCGACAAGGGCGCCAGCGCCCAGAGCTTCATCGAGCGCATGACCAACTTCGA |
| | | CAAGAACCTCCCCAACGAGAAGGTCCTCCCCAAGCACAGCCTCCTCTACGAGTACTTCACCGTCTACAACGAGCTCACCAAGGTCAAG |
| | | TACGTCACCGAGGGCATGCGCAAGCCCGCCTTCCTCAGCGGCGAGCAGAAGAAGGCCATCGTCGACCTCCTCTTCAAGACCAACCGCA |
| | | AGGTCACCGTCAAGCAGCTCAAGGAGGACTACTTCAAGAAGATCGAGTGCTTCGACAGCGTCGAGATCAGCGGCGTCGAGGACCGCTT |
| | | CAACGCCAGCCTCGGCACCTACCACGACCTCCTCAAGATCATCAAGGACAAGGACTTCCTCGACAACGAGGAGAACGAGGACATCCTC |
| | | GAGGACATCGTCCTCACCCTCACCCTCTTCGAGGACCGCGAGATGATCGAGGAGCGCCTCAAGACCTACGCCCACCTCTTCGACGACA |
| | | AGGTCATGAAGCAGCTCAAGCGCCGCCGCTACACCGGCTGGGGCCGCCTCAGCCGCAAGCTCATCAACGGCATCCGCGACAAGCAGAG |
| | | CGGCAAGACCATCCTCGACTTCCTCAAGAGCGACGGCTTCGCCAACCGCAACTTCATGCAGCTCATCCACGACGACAGCCTCACCTTC |
| | | AAGGAGGACATCCAGAAGGCCCAGGTCAGCGGCCAGGGCGACAGCCTCCACGAGCACATCGCCAACCTCGCCGGCAGCCCCGCCATCA |
| | | AGAAGGGCATCCTCCAGACCGTCAAGGTCGTCGACGAGCTCGTCAAGGTCATGGGCCGCCACAAGCCCGAGAACATCGTCATCGAGAT |
| | | GGCCCGCGAGAACCAGACCACCCAGAAGGGCCAGAAGAACAGCCGCGAGCGCATGAAGCGCATCGAGGAGGGCATCAAGGAGCTCGGC |
| | | AGCCAGATCCTCAAGGAGCACCCCGTCGAGAACACCCAGCTCCAGAACGAGAAGCTCTACCTCTACTACCTCCAGAACGGCCGCGACA |
| | | TGTACGTCGACCAGGAGCTCGACATCAACCGCCTCAGCGACTACGACGTCGACCACATCGTCCCCCAGAGCTTCCTCAAGGACGACAG |
| | | CATCGACAACAAGGTCCTCACCCGCAGCGACAAGAACCGCGGCAAGAGCGACAACGTCCCCAGCGAGGAGGTCGTCAAGAAGATGAAG |
| | | AACTACTGGCGCCAGCTCCTCAACGCCAAGCTCATCACCCAGCGCAAGTTCGACAACCTCACCAAGGCCGAGCGCGGCGGCCTCAGCG |
| | | AGCTCGACAAGGCCGGCTTCATCAAGCGCCAGCTCGTCGAGACCCGCCAGATCACCAAGCACGTCGCCCAGATCCTCGACAGCCGCAT |
| | | GAACACCAAGTACGACGAGAACGACAAGCTCATCCGCGAGGTCAAGGTCATCACCCTCAAGAGCAAGCTCGTCAGCGACTTCCGCAAG |
| | | GACTTCCAGTTCTACAAGGTCCGCGAGATCAACAACTACCACCACGCCCACGACGCCTACCTCAACGCCGTCGTCGGCACCGCCCTCA |
| | | TCAAGAAGTACCCCAAGCTCGAGAGCGAGTTCGTCTACGGCGACTACAAGGTCTACGACGTCCGCAAGATGATCGCCAAGAGCGAGCA |
| | | GGAGATCGGCAAGGCCACCGCCAAGTACTTCTTCTACAGCAACATCATGAACTTCTTCAAGACCGAGATCACCCTCGCCAACGGCGAG |
| | | ATCCGCAAGCGCCCCCTCATCGAGACCAACGGCGAGACCGGCGAGATCGTCTGGGACAAGGGCCGCGACTTCGCCACCGTCCGCAAGG |
| | | TCCTCAGCATGCCCCAGGTCAACATCGTCAAGAAGACCGAGGTCCAGACCGGCGGCTTCAGCAAGGAGAGCATCCTCCCCAAGCGCAA |
| | | CAGCGACAAGCTCATCGCCCGCAAGAAGGACTGGGACCCCAAGAAGTACGGCGGCTTCGACAGCCCCACCGTCGCCTACAGCGTCCTC |
| | | GTCGTCGCCAAGGTCGAGAAGGGCAAGAGCAAGAAGCTCAAGAGCGTCAAGGAGCTCCTCGGCATCACCATCATGGAGCGCAGCAGCT |
| | | TCGAGAAGAACCCCATCGACTTCCTCGAGGCCAAGGGCTACAAGGAGGTCAAGAAGGACCTCATCATCAAGCTCCCCAAGTACAGCCT |
| | | CTTCGAGCTCGAGAACGGCCGCAAGCGCATGCTCGCCAGCGCCGGCGAGCTCCAGAAGGGCAACGAGCTCGCCCTCCCCAGCAAGTAC |
| | | GTCAACTTCCTCTACCTCGCCAGCCACTACGAGAAGCTCAAGGGCAGCCCCGAGGACAACGAGCAGAAGCAGCTCTTCGTCGAGCAGC |
| | | ACAAGCACTACCTCGACGAGATCATCGAGCAGATCAGCGAGTTCAGCAAGCGCGTCATCCTCGCCGACGCCAACCTCGACAAGGTCCT |
| | | CAGCGCCTACAACAAGCACCGCGACAAGCCCATCCGCGAGCAGGCCGAGAACATCATCCACCTCTTCACCCTCACCAACCTCGGCGCC |
| | | CCCGCCGCCTTCAAGTACTTCGACACCACCATCGACCGCAAGCGCTACACCAGCACCAAGGAGGTCCTCGACGCCACCCTCATCCACC |
| | | AGAGCATCACCGGCCTCTACGAGACCCGCATCGACCTCAGCCAGCTCGGCGGCGACGGCGGCGGCAGCCCCAAGAAGAAGCGCAAGGT |
| | | CTAG |
| 818 | ORF encoding SpyCas9 | ATGGACAAGAAGTACAGCATCGGCCTGGACATCGGCACCAACAGCGTGGGCTGGGCCGTGATCACCGACGAGTACAAGGTGCCCAGCA |
| | | AGAAGTTCAAGGTGCTGGGCAACACCGACCGGCACAGCATCAAGAAGAACCTGATCGGCGCCCTGCTGTTCGACAGCGGCGAGACCGC |
| | | CGAGGCCACCCGGCTGAAGCGGACCGCCCGGCGGCGGTACACCCGGCGGAAGAACCGGATCTGCTACCTGCAGGAGATCTTCAGCAAC |
| | | GAGATGGCCAAGGTGGACGACAGCTTCTTCCACCGGCTGGAGGAGAGCTTCCTGGTGGAGGAGGACAAGAAGCACGAGCGGCACCCCA |
| | | TCTTCGGCAACATCGTGGACGAGGTGGCCTACCACGAGAAGTACCCCACCATCTACCACCTGCGGAAGAAGCTGGTGGACAGCACCGA |
| | | CAAGGCCGACCTGCGGCTGATCTACCTGGCCCTGGCCCACATGATCAAGTTCCGGGGCCACTTCCTGATCGAGGGCGACCTGAACCCC |
| | | GACAACAGCGACGTGGACAAGCTGTTCATCCAGCTGGTGCAGACCTACAACCAGCTGTTCGAGGAGAACCCCATCAACGCCAGCGGCG |
| | | TGGACGCCAAGGCCATCCTGAGCGCCCGGCTGAGCAAGAGCCGGCGGCTGGAGAACCTGATCGCCCAGCTGCCCGGCGAGAAGAAGAA |
| | | CGGCCTGTTCGGCAACCTGATCGCCCTGAGCCTGGGCCTGACCCCCAACTTCAAGAGCAACTTCGACCTGGCCGAGGACGCCAAGCTG |
| | | CAGCTGAGCAAGGACACCTACGACGACGACCTGGACAACCTGCTGGCCCAGATCGGCGACCAGTACGCCGACCTGTTCCTGGCCGCCA |
| | | AGAACCTGAGCGACGCCATCCTGCTGAGCGACATCCTGCGGGTGAACACCGAGATCACCAAGGCCCCCCTGAGCGCCAGCATGATCAA |
| | | GCGGTACGACGAGCACCACCAGGACCTGACCCTGCTGAAGGCCCTGGTGCGGCAGCAGCTGCCCGAGAAGTACAAGGAGATCTTCTTC |
| | | GACCAGAGCAAGAACGGCTACGCCGGCTACATCGACGGCGGCGCCAGCCAGGAGGAGTTCTACAAGTTCATCAAGCCCATCCTGGAGA |
| | | AGATGGACGGCACCGAGGAGCTGCTGGTGAAGCTGAACCGGGAGGACCTGCTGCGGAAGCAGCGGACCTTCGACAACGGCAGCATCCC |
| | | CCACCAGATCCACCTGGGCGAGCTGCACGCCATCCTGCGGCGGCAGGAGGACTTCTACCCCTTCCTGAAGGACAACCGGGAGAAGATC |
| | | GAGAAGATCCTGACCTTCCGGATCCCCTACTACGTGGGCCCCCTGGCCCGGGGCAACAGCCGGTTCGCCTGGATGACCCGGAAGAGCG |
| | | AGGAGACCATCACCCCCTGGAACTTCGAGGAGGTGGTGGACAAGGGCGCCAGCGCCCAGAGCTTCATCGAGCGGATGACCAACTTCGA |
| | | CAAGAACCTGCCCAACGAGAAGGTGCTGCCCAAGCACAGCCTGCTGTACGAGTACTTCACCGTGTACAACGAGCTGACCAAGGTGAAG |
| | | TACGTGACCGAGGGCATGCGGAAGCCCGCCTTCCTGAGCGGCGAGCAGAAGAAGGCCATCGTGGACCTGCTGTTCAAGACCAACCGGA |
| | | AGGTGACCGTGAAGCAGCTGAAGGAGGACTACTTCAAGAAGATCGAGTGCTTCGACAGCGTGGAGATCAGCGGCGTGGAGGACCGGTT |
| | | CAACGCCAGCCTGGGCACCTACCACGACCTGCTGAAGATCATCAAGGACAAGGACTTCCTGGACAACGAGGAGAACGAGGACATCCTG |
| | | GAGGACATCGTGCTGACCCTGACCCTGTTCGAGGACCGGGAGATGATCGAGGAGCGGCTGAAGACCTACGCCCACCTGTTCGACGACA |
| | | AGGTGATGAAGCAGCTGAAGCGGCGGCGGTACACCGGCTGGGGCCGGCTGAGCCGGAAGCTGATCAACGGCATCCGGGACAAGCAGAG |
| | | CGGCAAGACCATCCTGGACTTCCTGAAGAGCGACGGCTTCGCCAACCGGAACTTCATGCAGCTGATCCACGACGACAGCCTGACCTTC |
| | | AAGGAGGACATCCAGAAGGCCCAGGTGAGCGGCCAGGGCGACAGCCTGCACGAGCACATCGCCAACCTGGCCGGCAGCCCCGCCATCA |
| | | AGAAGGGCATCCTGCAGACCGTGAAGGTGGTGGACGAGCTGGTGAAGGTGATGGGCCGGCACAAGCCCGAGAACATCGTGATCGAGAT |
| | | GGCCCGGGAGAACCAGACCACCCAGAAGGGCCAGAAGAACAGCCGGGAGCGGATGAAGCGGATCGAGGAGGGCATCAAGGAGCTGGGC |
| | | AGCCAGATCCTGAAGGAGCACCCCGTGGAGAACACCCAGCTGCAGAACGAGAAGCTGTACCTGTACTACCTGCAGAACGGCCGGGACA |
| | | TGTACGTGGACCAGGAGCTGGACATCAACCGGCTGAGCGACTACGACGTGGACCACATCGTGCCCCAGAGCTTCCTGAAGGACGACAG |
| | | CATCGACAACAAGGTGCTGACCCGGAGCGACAAGAACCGGGGCAAGAGCGACAACGTGCCCAGCGAGGAGGTGGTGAAGAAGATGAAG |
| | | AACTACTGGCGGCAGCTGCTGAACGCCAAGCTGATCACCCAGCGGAAGTTCGACAACCTGACCAAGGCCGAGCGGGGCGGCCTGAGCG |
| | | AGCTGGACAAGGCCGGCTTCATCAAGCGGCAGCTGGTGGAGACCCGGCAGATCACCAAGCACGTGGCCCAGATCCTGGACAGCCGGAT |
| | | GAACACCAAGTACGACGAGAACGACAAGCTGATCCGGGAGGTGAAGGTGATCACCCTGAAGAGCAAGCTGGTGAGCGACTTCCGGAAG |
| | | GACTTCCAGTTCTACAAGGTGCGGGAGATCAACAACTACCACCACGCCCACGACGCCTACCTGAACGCCGTGGTGGGCACCGCCCTGA |
| | | TCAAGAAGTACCCCAAGCTGGAGAGCGAGTTCGTGTACGGCGACTACAAGGTGTACGACGTGCGGAAGATGATCGCCAAGAGCGAGCA |
| | | GGAGATCGGCAAGGCCACCGCCAAGTACTTCTTCTACAGCAACATCATGAACTTCTTCAAGACCGAGATCACCCTGGCCAACGGCGAG |
| | | ATCCGGAAGCGGCCCCTGATCGAGACCAACGGCGAGACCGGCGAGATCGTGTGGGACAAGGGCCGGGACTTCGCCACCGTGCGGAAGG |
| | | TGCTGAGCATGCCCCAGGTGAACATCGTGAAGAAGACCGAGGTGCAGACCGGCGGCTTCAGCAAGGAGAGCATCCTGCCCAAGCGGAA |
| | | CAGCGACAAGCTGATCGCCCGGAAGAAGGACTGGGACCCCAAGAAGTACGGCGGCTTCGACAGCCCCACCGTGGCCTACAGCGTGCTG |
| | | GTGGTGGCCAAGGTGGAGAAGGGCAAGAGCAAGAAGCTGAAGAGCGTGAAGGAGCTGCTGGGCATCACCATCATGGAGCGGAGCAGCT |
| | | TCGAGAAGAACCCCATCGACTTCCTGGAGGCCAAGGGCTACAAGGAGGTGAAGAAGGACCTGATCATCAAGCTGCCCAAGTACAGCCT |
| | | GTTCGAGCTGGAGAACGGCCGGAAGCGGATGCTGGCCAGCGCCGGCGAGCTGCAGAAGGGCAACGAGCTGGCCCTGCCCAGCAAGTAC |
| | | GTGAACTTCCTGTACCTGGCCAGCCACTACGAGAAGCTGAAGGGCAGCCCCGAGGACAACGAGCAGAAGCAGCTGTTCGTGGAGCAGC |
| | | ACAAGCACTACCTGGACGAGATCATCGAGCAGATCAGCGAGTTCAGCAAGCGGGTGATCCTGGCCGACGCCAACCTGGACAAGGTGCT |
| | | GAGCGCCTACAACAAGCACCGGGACAAGCCCATCCGGGAGCAGGCCGAGAACATCATCCACCTGTTCACCCTGACCAACCTGGGCGCC |
| | | CCCGCCGCCTTCAAGTACTTCGACACCACCATCGACCGGAAGCGGTACACCAGCACCAAGGAGGTGCTGGACGCCACCCTGATCCACC |
| | | AGAGCATCACCGGCCTGTACGAGACCCGGATCGACCTGAGCCAGCTGGGCGGCGACGGCGGCGGCAGCCCCAAGAAGAAGCGGAAGGT |
| | | GTGA |
| 819 | ORF encoding SpyCas9 | ATGGACAAGAAGTACAGCATCGGCCTGGACATCGGCACGAACAGCGTTGGCTGGGCTGTGATCACGGACGAGTACAAGGTTCCCTCAA |
| | | AGAAGTTCAAGGTGCTGGGCAACACGGACCGGCACAGCATCAAGAAGAATCTCATCGGTGCACTGCTGTTCGACAGCGGTGAGACGGC |
| | | CGAAGCCACGCGGCTGAAGCGGACGGCCCGCCGGCGGTACACGCGGCGGAAGAACCGGATCTGCTACCTGCAGGAGATCTTCAGCAAC |
| | | GAGATGGCCAAGGTGGACGACAGCTTCTTCCACCGGCTGGAGGAGAGCTTCCTGGTGGAGGAGGACAAGAAGCACGAGCGGCACCCCA |
| | | TCTTCGGCAACATCGTGGACGAAGTCGCCTACCACGAGAAGTACCCCACCATCTACCACCTGCGGAAGAAGCTGGTGGACTCGACTGA |
| | | CAAGGCCGACCTGCGGCTGATCTACCTGGCACTGGCCCACATGATAAAGTTCCGGGGCCACTTCCTGATCGAGGGCGACCTGAACCCT |
| | | GACAACAGCGACGTGGACAAGCTGTTCATCCAGCTGGTGCAGACCTACAACCAGCTGTTCGAGGAGAACCCCATCAACGCCAGCGGCG |
| | | TGGACGCCAAGGCCATCCTCAGCGCCCGCCTCAGCAAGAGCCGGCGGCTGGAGAATCTCATCGCCCAGCTTCCAGGTGAGAAGAAGAA |
| | | TGGGCTGTTCGGCAATCTCATCGCACTCAGCCTGGGCCTGACTCCCAACTTCAAGAGCAACTTCGACCTGGCCGAGGACGCCAAGCTG |
| | | CAGCTCAGCAAGGACACCTACGACGACGACCTGGACAATCTCCTGGCCCAGATCGGCGACCAGTACGCCGACCTGTTCCTGGCTGCCA |
| | | AGAATCTCAGCGACGCCATCCTGCTCAGCGACATCCTGCGGGTGAACACAGAGATCACGAAGGCCCCCCTCAGCGCCAGCATGATAAA |
| | | GCGGTACGACGAGCACCACCAGGACCTGACGCTGCTGAAGGCACTGGTGCGGCAGCAGCTTCCAGAGAAGTACAAGGAGATCTTCTTC |
| | | GACCAGAGCAAGAATGGGTACGCCGGGTACATCGACGGTGGTGCCAGCCAGGAGGAGTTCTACAAGTTCATCAAGCCCATCCTGGAGA |
| | | AGATGGACGGCACAGAGGAGCTGCTGGTGAAGCTGAACAGGGAGGACCTGCTGCGGAAGCAGCGGACGTTCGACAATGGGAGCATCCC |
| | | CCACCAGATCCACCTGGGTGAGCTGCACGCCATCCTGCGGCGGCAGGAGGACTTCTACCCCTTCCTGAAGGACAACAGGGAGAAGATC |
| | | GAGAAGATCCTGACGTTCCGGATCCCCTACTACGTTGGCCCCCTGGCCCGCGGCAACAGCCGGTTCGCCTGGATGACGCGGAAGAGCG |
| | | AGGAGACGATCACTCCCTGGAACTTCGAGGAAGTCGTGGACAAGGGTGCCAGCGCCCAGAGCTTCATCGAGCGGATGACGAACTTCGA |
| | | CAAGAATCTTCCAAACGAGAAGGTGCTTCCAAAGCACAGCCTGCTGTACGAGTACTTCACGGTGTACAACGAGCTGACGAAGGTGAAG |
| | | TACGTGACAGAGGGCATGCGGAAGCCCGCCTTCCTCAGCGGTGAGCAGAAGAAGGCCATCGTGGACCTGCTGTTCAAGACGAACCGGA |
| | | AGGTGACGGTGAAGCAGCTGAAGGAGGACTACTTCAAGAAGATCGAGTGCTTCGACAGCGTGGAGATCAGCGGCGTGGAGGACCGGTT |
| | | CAACGCCAGCCTGGGCACCTACCACGACCTGCTGAAGATCATCAAGGACAAGGACTTCCTGGACAACGAGGAGAACGAGGACATCCTG |
| | | GAGGACATCGTGCTGACGCTGACGCTGTTCGAGGACAGGGAGATGATAGAGGAGCGGCTGAAGACCTACGCCCACCTGTTCGACGACA |
| | | AGGTGATGAAGCAGCTGAAGCGGCGGCGGTACACGGGCTGGGGCCGGCTCAGCCGGAAGCTGATCAATGGGATCCGAGACAAGCAGAG |
| | | CGGCAAGACGATCCTGGACTTCCTGAAGAGCGACGGCTTCGCCAACCGGAACTTCATGCAGCTGATCCACGACGACAGCCTGACGTTC |
| | | AAGGAGGACATCCAGAAGGCCCAGGTCAGCGGCCAGGGCGACAGCCTGCACGAGCACATCGCCAATCTCGCCGGGAGCCCCGCCATCA |
| | | AGAAGGGGATCCTGCAGACGGTGAAGGTGGTGGACGAGCTGGTGAAGGTGATGGGCCGGCACAAGCCAGAGAACATCGTGATCGAGAT |
| | | GGCCAGGGAGAACCAGACGACTCAAAAGGGGCAGAAGAACAGCAGGGAGCGGATGAAGCGGATCGAGGAGGGCATCAAGGAGCTGGGC |
| | | AGCCAGATCCTGAAGGAGCACCCCGTGGAGAACACTCAACTGCAGAACGAGAAGCTGTACCTGTACTACCTGCAGAATGGGCGAGACA |
| | | TGTACGTGGACCAGGAGCTGGACATCAACCGGCTCAGCGACTACGACGTGGACCACATCGTTCCCCAGAGCTTCCTGAAGGACGACAG |
| | | CATCGACAACAAGGTGCTGACGCGGAGCGACAAGAACCGGGGCAAGAGCGACAACGTTCCCTCAGAGGAAGTCGTGAAGAAGATGAAG |
| | | AACTACTGGCGGCAGCTGCTGAACGCCAAGCTGATCACTCAACGGAAGTTCGACAATCTCACGAAGGCCGAGCGGGGTGGCCTCAGCG |
| | | AGCTGGACAAGGCCGGGTTCATCAAGCGGCAGCTGGTGGAGACGCGGCAGATCACGAAGCACGTGGCCCAGATCCTGGACAGCCGGAT |
| | | GAACACGAAGTACGACGAGAACGACAAGCTGATCAGGGAAGTCAAGGTGATCACGCTGAAGAGCAAGCTGGTCAGCGACTTCCGGAAG |
| | | GACTTCCAGTTCTACAAGGTGAGGGAGATCAACAACTACCACCACGCCCACGACGCCTACCTGAACGCTGTGGTTGGCACGGCACTGA |
| | | TCAAGAAGTACCCCAAGCTGGAGAGCGAGTTCGTGTACGGCGACTACAAGGTGTACGACGTGCGGAAGATGATAGCCAAGAGCGAGCA |
| | | GGAGATCGGCAAGGCCACGGCCAAGTACTTCTTCTACAGCAACATCATGAACTTCTTCAAGACAGAGATCACGCTGGCCAATGGTGAG |
| | | ATCCGGAAGCGGCCCCTGATCGAGACGAATGGTGAGACGGGTGAGATCGTGTGGGACAAGGGGCGAGACTTCGCCACGGTGCGGAAGG |
| | | TGCTCAGCATGCCCCAGGTGAACATCGTGAAGAAGACAGAAGTCCAGACGGGTGGCTTCAGCAAGGAGAGCATCCTTCCAAAGCGGAA |
| | | CAGCGACAAGCTGATCGCCCGCAAGAAGGACTGGGACCCCAAGAAGTACGGTGGCTTCGACAGCCCCACCGTGGCCTACAGCGTGCTG |
| | | GTGGTGGCCAAGGTGGAGAAGGGGAAGAGCAAGAAGCTGAAGAGCGTGAAGGAGCTGCTGGGCATCACGATCATGGAGCGGAGCAGCT |
| | | TCGAGAAGAACCCCATCGACTTCCTGGAAGCCAAGGGGTACAAGGAAGTCAAGAAGGACCTGATCATCAAGCTTCCAAAGTACAGCCT |
| | | GTTCGAGCTGGAGAATGGGCGGAAGCGGATGCTGGCCAGCGCCGGTGAGCTGCAGAAGGGGAACGAGCTGGCACTTCCCTCAAAGTAC |
| | | GTGAACTTCCTGTACCTGGCCAGCCACTACGAGAAGCTGAAGGGGAGCCCAGAGGACAACGAGCAGAAGCAGCTGTTCGTGGAGCAGC |
| | | ACAAGCACTACCTGGACGAGATCATCGAGCAGATCAGCGAGTTCAGCAAGCGGGTGATCCTGGCCGACGCCAATCTCGACAAGGTGCT |
| | | CAGCGCCTACAACAAGCACCGAGACAAGCCCATCAGGGAGCAGGCCGAGAACATCATCCACCTGTTCACGCTGACGAATCTCGGTGCC |
| | | CCCGCTGCCTTCAAGTACTTCGACACGACGATCGACCGGAAGCGGTACACGTCGACTAAGGAAGTCCTGGACGCCACGCTGATCCACC |
| | | AGAGCATCACGGGCCTGTACGAGACGCGGATCGACCTCAGCCAGCTGGGTGGCGACGGTGGTGGCAGCCCCAAGAAGAAGCGGAAGGT |
| | | GTAG |
| 820 | mRNA encoding SpyCas9 | ATGGACAAGAAGTACAGCATCGGACTGGACATCGGAACAAACAGCGTCGGATGGGCAGTCATCACAGACGAATACAAGGTCCCGAGCA |
| | | AGAAGTTCAAGGTCCTGGGAAACACAGACAGACACAGCATCAAGAAGAACCTGATCGGAGCACTGCTGTTCGACAGCGGAGAAACAGC |
| | | AGAAGCAACAAGACTGAAGAGAACAGCAAGAAGAAGATACACAAGAAGAAAGAACAGAATCTGCTACCTGCAGGAAATCTTCAGCAAC |
| | | GAAATGGCAAAGGTCGACGACAGCTTCTTCCACAGACTGGAAGAAAGCTTCCTGGTCGAAGAAGACAAGAAGCACGAAAGACACCCGA |
| | | TCTTCGGAAACATCGTCGACGAAGTCGCATACCACGAAAAGTACCCGACAATCTACCACCTGAGAAAGAAGCTGGTCGACAGCACAGA |
| | | CAAGGCAGACCTGAGACTGATCTACCTGGCACTGGCACACATGATCAAGTTCAGAGGACACTTCCTGATCGAAGGAGACCTGAACCCG |
| | | GACAACAGCGACGTCGACAAGCTGTTCATCCAGCTGGTCCAGACATACAACCAGCTGTTCGAAGAAAACCCGATCAACGCAAGCGGAG |
| | | TCGACGCAAAGGCAATCCTGAGCGCAAGACTGAGCAAGAGCAGAAGACTGGAAAACCTGATCGCACAGCTGCCGGGAGAAAAGAAGAA |
| | | CGGACTGTTCGGAAACCTGATCGCACTGAGCCTGGGACTGACACCGAACTTCAAGAGCAACTTCGACCTGGCAGAAGACGCAAAGCTG |
| | | CAGCTGAGCAAGGACACATACGACGACGACCTGGACAACCTGCTGGCACAGATCGGAGACCAGTACGCAGACCTGTTCCTGGCAGCAA |
| | | AGAACCTGAGCGACGCAATCCTGCTGAGCGACATCCTGAGAGTCAACACAGAAATCACAAAGGCACCGCTGAGCGCAAGCATGATCAA |
| | | GAGATACGACGAACACCACCAGGACCTGACACTGCTGAAGGCACTGGTCAGACAGCAGCTGCCGGAAAAGTACAAGGAAATCTTCTTC |
| | | GACCAGAGCAAGAACGGATACGCAGGATACATCGACGGAGGAGCAAGCCAGGAAGAATTCTACAAGTTCATCAAGCCGATCCTGGAAA |
| | | AGATGGACGGAACAGAAGAACTGCTGGTCAAGCTGAACAGAGAAGACCTGCTGAGAAAGCAGAGAACATTCGACAACGGAAGCATCCC |
| | | GCACCAGATCCACCTGGGAGAACTGCACGCAATCCTGAGAAGACAGGAAGACTTCTACCCGTTCCTGAAGGACAACAGAGAAAAGATC |
| | | GAAAAGATCCTGACATTCAGAATCCCGTACTACGTCGGACCGCTGGCAAGAGGAAACAGCAGATTCGCATGGATGACAAGAAAGAGCG |
| | | AAGAAACAATCACACCGTGGAACTTCGAAGAAGTCGTCGACAAGGGAGCAAGCGCACAGAGCTTCATCGAAAGAATGACAAACTTCGA |
| | | CAAGAACCTGCCGAACGAAAAGGTCCTGCCGAAGCACAGCCTGCTGTACGAATACTTCACAGTCTACAACGAACTGACAAAGGTCAAG |
| | | TACGTCACAGAAGGAATGAGAAAGCCGGCATTCCTGAGCGGAGAACAGAAGAAGGCAATCGTCGACCTGCTGTTCAAGACAAACAGAA |
| | | AGGTCACAGTCAAGCAGCTGAAGGAAGACTACTTCAAGAAGATCGAATGCTTCGACAGCGTCGAAATCAGCGGAGTCGAAGACAGATT |
| | | CAACGCAAGCCTGGGAACATACCACGACCTGCTGAAGATCATCAAGGACAAGGACTTCCTGGACAACGAAGAAAACGAAGACATCCTG |
| | | GAAGACATCGTCCTGACACTGACACTGTTCGAAGACAGAGAAATGATCGAAGAAAGACTGAAGACATACGCACACCTGTTCGACGACA |
| | | AGGTCATGAAGCAGCTGAAGAGAAGAAGATACACAGGATGGGGAAGACTGAGCAGAAAGCTGATCAACGGAATCAGAGACAAGCAGAG |
| | | CGGAAAGACAATCCTGGACTTCCTGAAGAGCGACGGATTCGCAAACAGAAACTTCATGCAGCTGATCCACGACGACAGCCTGACATTC |
| | | AAGGAAGACATCCAGAAGGCACAGGTCAGCGGACAGGGAGACAGCCTGCACGAACACATCGCAAACCTGGCAGGAAGCCCGGCAATCA |
| | | AGAAGGGAATCCTGCAGACAGTCAAGGTCGTCGACGAACTGGTCAAGGTCATGGGAAGACACAAGCCGGAAAACATCGTCATCGAAAT |
| | | GGCAAGAGAAAACCAGACAACACAGAAGGGACAGAAGAACAGCAGAGAAAGAATGAAGAGAATCGAAGAAGGAATCAAGGAACTGGGA |
| | | AGCCAGATCCTGAAGGAACACCCGGTCGAAAACACACAGCTGCAGAACGAAAAGCTGTACCTGTACTACCTGCAGAACGGAAGAGACA |
| | | TGTACGTCGACCAGGAACTGGACATCAACAGACTGAGCGACTACGACGTCGACCACATCGTCCCGCAGAGCTTCCTGAAGGACGACAG |
| | | CATCGACAACAAGGTCCTGACAAGAAGCGACAAGAACAGAGGAAAGAGCGACAACGTCCCGAGCGAAGAAGTCGTCAAGAAGATGAAG |
| | | AACTACTGGAGACAGCTGCTGAACGCAAAGCTGATCACACAGAGAAAGTTCGACAACCTGACAAAGGCAGAGAGAGGAGGACTGAGCG |
| | | AACTGGACAAGGCAGGATTCATCAAGAGACAGCTGGTCGAAACAAGACAGATCACAAAGCACGTCGCACAGATCCTGGACAGCAGAAT |
| | | GAACACAAAGTACGACGAAAACGACAAGCTGATCAGAGAAGTCAAGGTCATCACACTGAAGAGCAAGCTGGTCAGCGACTTCAGAAAG |
| | | GACTTCCAGTTCTACAAGGTCAGAGAAATCAACAACTACCACCACGCACACGACGCATACCTGAACGCAGTCGTCGGAACAGCACTGA |
| | | TCAAGAAGTACCCGAAGCTGGAAAGCGAATTCGTCTACGGAGACTACAAGGTCTACGACGTCAGAAAGATGATCGCAAAGAGCGAACA |
| | | GGAAATCGGAAAGGCAACAGCAAAGTACTTCTTCTACAGCAACATCATGAACTTCTTCAAGACAGAAATCACACTGGCAAACGGAGAA |
| | | ATCAGAAAGAGACCGCTGATCGAAACAAACGGAGAAACAGGAGAAATCGTCTGGGACAAGGGAAGAGACTTCGCAACAGTCAGAAAGG |
| | | TCCTGAGCATGCCGCAGGTCAACATCGTCAAGAAGACAGAAGTCCAGACAGGAGGATTCAGCAAGGAAAGCATCCTGCCGAAGAGAAA |
| | | CAGCGACAAGCTGATCGCAAGAAAGAAGGACTGGGACCCGAAGAAGTACGGAGGATTCGACAGCCCGACAGTCGCATACAGCGTCCTG |
| | | GTCGTCGCAAAGGTCGAAAAGGGAAAGAGCAAGAAGCTGAAGAGCGTCAAGGAACTGCTGGGAATCACAATCATGGAAAGAAGCAGCT |
| | | TCGAAAAGAACCCGATCGACTTCCTGGAAGCAAAGGGATACAAGGAAGTCAAGAAGGACCTGATCATCAAGCTGCCGAAGTACAGCCT |
| | | GTTCGAACTGGAAAACGGAAGAAAGAGAATGCTGGCAAGCGCAGGAGAACTGCAGAAGGGAAACGAACTGGCACTGCCGAGCAAGTAC |
| | | GTCAACTTCCTGTACCTGGCAAGCCACTACGAAAAGCTGAAGGGAAGCCCGGAAGACAACGAACAGAAGCAGCTGTTCGTCGAACAGC |
| | | ACAAGCACTACCTGGACGAAATCATCGAACAGATCAGCGAATTCAGCAAGAGAGTCATCCTGGCAGACGCAAACCTGGACAAGGTCCT |
| | | GAGCGCATACAACAAGCACAGAGACAAGCCGATCAGAGAACAGGCAGAAAACATCATCCACCTGTTCACACTGACAAACCTGGGAGCA |
| | | CCGGCAGCATTCAAGTACTTCGACACAACAATCGACAGAAAGAGATACACAAGCACAAAGGAAGTCCTGGACGCAACACTGATCCACC |
| | | AGAGCATCACAGGACTGTACGAAACAAGAATCGACCTGAGCCAGCTGGGAGGAGACGGAGGAGGAAGCCCGAAGAAGAAGAGAAAGGT |
| | | CTAGCTAGCCATCACATTTAAAAGCATCTCAGCCTACCATGAGAATAAGAGAAAGAAAATGAAGATCAATAGCTTATTCATCTCTTTT |
| | | TCTTTTTCGTTGGTGTAAAGCCAACACCCTGTCTAAAAAACATAAATTTCTTTAATCATTTTGCCTCTTTTCTCTGTGCTTCAATTAA |
| | | TAAAAAATGGAAAGAACCTCGAGAAAAAAAAAAAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAATCTAG |
| 821 | mRNA encoding SpyCas9 base editor | GGGAAGCTCAGAATAAACGCTCAACTTTGGCCGGATCTGCCACCATGGAGGCCTCCCCCGCCTCCGGCCCCCGGCACCTGATGGACCC |
| | | CCACATCTTCACCTCCAACTTCAACAACGGCATCGGCCGGCACAAGACCTACCTGTGCTACGAGGTGGAGCGGCTGGACAACGGCACC |
| | | TCCGTGAAGATGGACCAGCACCGGGGCTTCCTGCACAACCAGGCCAAGAACCTGCTGTGCGGCTTCTACGGCCGGCACGCCGAGCTGC |
| | | GGTTCCTGGACCTGGTGCCCTCCCTGCAGCTGGACCCCGCCCAGATCTACCGGGTGACCTGGTTCATCTCCTGGTCCCCCTGCTTCTC |
| | | CTGGGGCTGCGCCGGCGAGGTGCGGGCCTTCCTGCAGGAGAACACCCACGTGCGGCTGCGGATCTTCGCCGCCCGGATCTACGACTAC |
| | | GACCCCCTGTACAAGGAGGCCCTGCAGATGCTGCGGGACGCCGGCGCCCAGGTGTCCATCATGACCTACGACGAGTTCAAGCACTGCT |
| | | GGGACACCTTCGTGGACCACCAGGGCTGCCCCTTCCAGCCCTGGGACGGCCTGGACGAGCACTCCCAGGCCCTGTCCGGCCGGCTGCG |
| | | GGCCATCCTGCAGAACCAGGGCAACTCCGGCTCCGAGACCCCCGGCACCTCCGAGTCCGCCACCCCCGAGTCCGACAAGAAGTACTCC |
| | | ATCGGCCTGGCCATCGGCACCAACTCCGTGGGCTGGGCCGTGATCACCGACGAGTACAAGGTGCCCTCCAAGAAGTTCAAGGTGCTGG |
| | | GCAACACCGACCGGCACTCCATCAAGAAGAACCTGATCGGCGCCCTGCTGTTCGACTCCGGCGAGACCGCCGAGGCCACCCGGCTGAA |
| | | GCGGACCGCCCGGCGGCGGTACACCCGGCGGAAGAACCGGATCTGCTACCTGCAGGAGATCTTCTCCAACGAGATGGCCAAGGTGGAC |
| | | GACTCCTTCTTCCACCGGCTGGAGGAGTCCTTCCTGGTGGAGGAGGACAAGAAGCACGAGCGGCACCCCATCTTCGGCAACATCGTGG |
| | | ACGAGGTGGCCTACCACGAGAAGTACCCCACCATCTACCACCTGCGGAAGAAGCTGGTGGACTCCACCGACAAGGCCGACCTGCGGCT |
| | | GATCTACCTGGCCCTGGCCCACATGATCAAGTTCCGGGGCCACTTCCTGATCGAGGGCGACCTGAACCCCGACAACTCCGACGTGGAC |
| | | AAGCTGTTCATCCAGCTGGTGCAGACCTACAACCAGCTGTTCGAGGAGAACCCCATCAACGCCTCCGGCGTGGACGCCAAGGCCATCC |
| | | TGTCCGCCCGGCTGTCCAAGTCCCGGCGGCTGGAGAACCTGATCGCCCAGCTGCCCGGCGAGAAGAAGAACGGCCTGTTCGGCAACCT |
| | | GATCGCCCTGTCCCTGGGCCTGACCCCCAACTTCAAGTCCAACTTCGACCTGGCCGAGGACGCCAAGCTGCAGCTGTCCAAGGACACC |
| | | TACGACGACGACCTGGACAACCTGCTGGCCCAGATCGGCGACCAGTACGCCGACCTGTTCCTGGCCGCCAAGAACCTGTCCGACGCCA |
| | | TCCTGCTGTCCGACATCCTGCGGGTGAACACCGAGATCACCAAGGCCCCCCTGTCCGCCTCCATGATCAAGCGGTACGACGAGCACCA |
| | | CCAGGACCTGACCCTGCTGAAGGCCCTGGTGCGGCAGCAGCTGCCCGAGAAGTACAAGGAGATCTTCTTCGACCAGTCCAAGAACGGC |
| | | TACGCCGGCTACATCGACGGCGGCGCCTCCCAGGAGGAGTTCTACAAGTTCATCAAGCCCATCCTGGAGAAGATGGACGGCACCGAGG |
| | | AGCTGCTGGTGAAGCTGAACCGGGAGGACCTGCTGCGGAAGCAGCGGACCTTCGACAACGGCTCCATCCCCCACCAGATCCACCTGGG |
| | | CGAGCTGCACGCCATCCTGCGGCGGCAGGAGGACTTCTACCCCTTCCTGAAGGACAACCGGGAGAAGATCGAGAAGATCCTGACCTTC |
| | | CGGATCCCCTACTACGTGGGCCCCCTGGCCCGGGGCAACTCCCGGTTCGCCTGGATGACCCGGAAGTCCGAGGAGACCATCACCCCCT |
| | | GGAACTTCGAGGAGGTGGTGGACAAGGGCGCCTCCGCCCAGTCCTTCATCGAGCGGATGACCAACTTCGACAAGAACCTGCCCAACGA |
| | | GAAGGTGCTGCCCAAGCACTCCCTGCTGTACGAGTACTTCACCGTGTACAACGAGCTGACCAAGGTGAAGTACGTGACCGAGGGCATG |
| | | CGGAAGCCCGCCTTCCTGTCCGGCGAGCAGAAGAAGGCCATCGTGGACCTGCTGTTCAAGACCAACCGGAAGGTGACCGTGAAGCAGC |
| | | TGAAGGAGGACTACTTCAAGAAGATCGAGTGCTTCGACTCCGTGGAGATCTCCGGCGTGGAGGACCGGTTCAACGCCTCCCTGGGCAC |
| | | CTACCACGACCTGCTGAAGATCATCAAGGACAAGGACTTCCTGGACAACGAGGAGAACGAGGACATCCTGGAGGACATCGTGCTGACC |
| | | CTGACCCTGTTCGAGGACCGGGAGATGATCGAGGAGCGGCTGAAGACCTACGCCCACCTGTTCGACGACAAGGTGATGAAGCAGCTGA |
| | | AGCGGCGGCGGTACACCGGCTGGGGCCGGCTGTCCCGGAAGCTGATCAACGGCATCCGGGACAAGCAGTCCGGCAAGACCATCCTGGA |
| | | CTTCCTGAAGTCCGACGGCTTCGCCAACCGGAACTTCATGCAGCTGATCCACGACGACTCCCTGACCTTCAAGGAGGACATCCAGAAG |
| | | GCCCAGGTGTCCGGCCAGGGCGACTCCCTGCACGAGCACATCGCCAACCTGGCCGGCTCCCCCGCCATCAAGAAGGGCATCCTGCAGA |
| | | CCGTGAAGGTGGTGGACGAGCTGGTGAAGGTGATGGGCCGGCACAAGCCCGAGAACATCGTGATCGAGATGGCCCGGGAGAACCAGAC |
| | | CACCCAGAAGGGCCAGAAGAACTCCCGGGAGCGGATGAAGCGGATCGAGGAGGGCATCAAGGAGCTGGGCTCCCAGATCCTGAAGGAG |
| | | CACCCCGTGGAGAACACCCAGCTGCAGAACGAGAAGCTGTACCTGTACTACCTGCAGAACGGCCGGGACATGTACGTGGACCAGGAGC |
| | | TGGACATCAACCGGCTGTCCGACTACGACGTGGACCACATCGTGCCCCAGTCCTTCCTGAAGGACGACTCCATCGACAACAAGGTGCT |
| | | GACCCGGTCCGACAAGAACCGGGGCAAGTCCGACAACGTGCCCTCCGAGGAGGTGGTGAAGAAGATGAAGAACTACTGGCGGCAGCTG |
| | | CTGAACGCCAAGCTGATCACCCAGCGGAAGTTCGACAACCTGACCAAGGCCGAGCGGGGCGGCCTGTCCGAGCTGGACAAGGCCGGCT |
| | | TCATCAAGCGGCAGCTGGTGGAGACCCGGCAGATCACCAAGCACGTGGCCCAGATCCTGGACTCCCGGATGAACACCAAGTACGACGA |
| | | GAACGACAAGCTGATCCGGGAGGTGAAGGTGATCACCCTGAAGTCCAAGCTGGTGTCCGACTTCCGGAAGGACTTCCAGTTCTACAAG |
| | | GTGCGGGAGATCAACAACTACCACCACGCCCACGACGCCTACCTGAACGCCGTGGTGGGCACCGCCCTGATCAAGAAGTACCCCAAGC |
| | | TGGAGTCCGAGTTCGTGTACGGCGACTACAAGGTGTACGACGTGCGGAAGATGATCGCCAAGTCCGAGCAGGAGATCGGCAAGGCCAC |
| | | CGCCAAGTACTTCTTCTACTCCAACATCATGAACTTCTTCAAGACCGAGATCACCCTGGCCAACGGCGAGATCCGGAAGCGGCCCCTG |
| | | ATCGAGACCAACGGCGAGACCGGCGAGATCGTGTGGGACAAGGGCCGGGACTTCGCCACCGTGCGGAAGGTGCTGTCCATGCCCCAGG |
| | | TGAACATCGTGAAGAAGACCGAGGTGCAGACCGGCGGCTTCTCCAAGGAGTCCATCCTGCCCAAGCGGAACTCCGACAAGCTGATCGC |
| | | CCGGAAGAAGGACTGGGACCCCAAGAAGTACGGCGGCTTCGACTCCCCCACCGTGGCCTACTCCGTGCTGGTGGTGGCCAAGGTGGAG |
| | | AAGGGCAAGTCCAAGAAGCTGAAGTCCGTGAAGGAGCTGCTGGGCATCACCATCATGGAGCGGTCCTCCTTCGAGAAGAACCCCATCG |
| | | ACTTCCTGGAGGCCAAGGGCTACAAGGAGGTGAAGAAGGACCTGATCATCAAGCTGCCCAAGTACTCCCTGTTCGAGCTGGAGAACGG |
| | | CCGGAAGCGGATGCTGGCCTCCGCCGGCGAGCTGCAGAAGGGCAACGAGCTGGCCCTGCCCTCCAAGTACGTGAACTTCCTGTACCTG |
| | | GCCTCCCACTACGAGAAGCTGAAGGGCTCCCCCGAGGACAACGAGCAGAAGCAGCTGTTCGTGGAGCAGCACAAGCACTACCTGGACG |
| | | AGATCATCGAGCAGATCTCCGAGTTCTCCAAGCGGGTGATCCTGGCCGACGCCAACCTGGACAAGGTGCTGTCCGCCTACAACAAGCA |
| | | CCGGGACAAGCCCATCCGGGAGCAGGCCGAGAACATCATCCACCTGTTCACCCTGACCAACCTGGGCGCCCCCGCCGCCTTCAAGTAC |
| | | TTCGACACCACCATCGACCGGAAGCGGTACACCTCCACCAAGGAGGTGCTGGACGCCACCCTGATCCACCAGTCCATCACCGGCCTGT |
| | | ACGAGACCCGGATCGACCTGTCCCAGCTGGGCGGCGACGGCGGCGGCTCCCCCAAGAAGAAGCGGAAGGTGTGACTAGCACCAGCCTC |
| | | AAGAACACCCGAATGGAGTCTCTAAGCTACATAATACCAACTTACACTTTACAAAATGTTGTCCCCCAAAATGTAGCCATTCGTATCT |
| | | GCTCCTAATAAAAAGAAAGTTTCTTCACATTCTCTCGAGAAAAAAAAAAAATGGAAAAAAAAAAAACGGAAAAAAAAAAAAGGTAAAA |
| | | AAAAAAAATATAAAAAAAAAAAACATAAAAAAAAAAAACGAAAAAAAAAAAACGTAAAAAAAAAAAACTCAAAAAAAAAAAAGATAAA |
| | | AAAAAAAAACCTAAAAAAAAAAAATGTAAAAAAAAAAAAGGGAAAAAAAAAAAACGCAAAAAAAAAAAACACAAAAAAAAAAAATGCA |
| | | AAAAAAAAAAATCGAAAAAAAAAAAATCTAAAAAAAAAAAACGAAAAAAAAAAAACCCAAAAAAAAAAAAGACAAAAAAAAAAAATAG |
| | | AAAAAAAAAAAAGTTAAAAAAAAAAAACTGAAAAAAAAAAAATTTAAAAAAAAAAAATCTAG |
| 822 | mRNA encoding SpyCas9 base editor with | GGGAAGCTCAGAATAAACGCTCAACTTTGGCCGGATCTGCCACCATGGAGGCCTCCCCCGCCTCCGGCCCCCGGCACCTGATGGACCC |
| | Hibit tag | CCACATCTTCACCTCCAACTTCAACAACGGCATCGGCCGGCACAAGACCTACCTGTGCTACGAGGTGGAGCGGCTGGACAACGGCACC |
| | | TCCGTGAAGATGGACCAGCACCGGGGCTTCCTGCACAACCAGGCCAAGAACCTGCTGTGCGGCTTCTACGGCCGGCACGCCGAGCTGC |
| | | GGTTCCTGGACCTGGTGCCCTCCCTGCAGCTGGACCCCGCCCAGATCTACCGGGTGACCTGGTTCATCTCCTGGTCCCCCTGCTTCTC |
| | | CTGGGGCTGCGCCGGCGAGGTGCGGGCCTTCCTGCAGGAGAACACCCACGTGCGGCTGCGGATCTTCGCCGCCCGGATCTACGACTAC |
| | | GACCCCCTGTACAAGGAGGCCCTGCAGATGCTGCGGGACGCCGGCGCCCAGGTGTCCATCATGACCTACGACGAGTTCAAGCACTGCT |
| | | GGGACACCTTCGTGGACCACCAGGGCTGCCCCTTCCAGCCCTGGGACGGCCTGGACGAGCACTCCCAGGCCCTGTCCGGCCGGCTGCG |
| | | GGCCATCCTGCAGAACCAGGGCAACTCCGGCTCCGAGACCCCCGGCACCTCCGAGTCCGCCACCCCCGAGTCCGACAAGAAGTACTCC |
| | | ATCGGCCTGGCCATCGGCACCAACTCCGTGGGCTGGGCCGTGATCACCGACGAGTACAAGGTGCCCTCCAAGAAGTTCAAGGTGCTGG |
| | | GCAACACCGACCGGCACTCCATCAAGAAGAACCTGATCGGCGCCCTGCTGTTCGACTCCGGCGAGACCGCCGAGGCCACCCGGCTGAA |
| | | GCGGACCGCCCGGCGGCGGTACACCCGGCGGAAGAACCGGATCTGCTACCTGCAGGAGATCTTCTCCAACGAGATGGCCAAGGTGGAC |
| | | GACTCCTTCTTCCACCGGCTGGAGGAGTCCTTCCTGGTGGAGGAGGACAAGAAGCACGAGCGGCACCCCATCTTCGGCAACATCGTGG |
| | | ACGAGGTGGCCTACCACGAGAAGTACCCCACCATCTACCACCTGCGGAAGAAGCTGGTGGACTCCACCGACAAGGCCGACCTGCGGCT |
| | | GATCTACCTGGCCCTGGCCCACATGATCAAGTTCCGGGGCCACTTCCTGATCGAGGGCGACCTGAACCCCGACAACTCCGACGTGGAC |
| | | AAGCTGTTCATCCAGCTGGTGCAGACCTACAACCAGCTGTTCGAGGAGAACCCCATCAACGCCTCCGGCGTGGACGCCAAGGCCATCC |
| | | TGTCCGCCCGGCTGTCCAAGTCCCGGCGGCTGGAGAACCTGATCGCCCAGCTGCCCGGCGAGAAGAAGAACGGCCTGTTCGGCAACCT |
| | | GATCGCCCTGTCCCTGGGCCTGACCCCCAACTTCAAGTCCAACTTCGACCTGGCCGAGGACGCCAAGCTGCAGCTGTCCAAGGACACC |
| | | TACGACGACGACCTGGACAACCTGCTGGCCCAGATCGGCGACCAGTACGCCGACCTGTTCCTGGCCGCCAAGAACCTGTCCGACGCCA |
| | | TCCTGCTGTCCGACATCCTGCGGGTGAACACCGAGATCACCAAGGCCCCCCTGTCCGCCTCCATGATCAAGCGGTACGACGAGCACCA |
| | | CCAGGACCTGACCCTGCTGAAGGCCCTGGTGCGGCAGCAGCTGCCCGAGAAGTACAAGGAGATCTTCTTCGACCAGTCCAAGAACGGC |
| | | TACGCCGGCTACATCGACGGCGGCGCCTCCCAGGAGGAGTTCTACAAGTTCATCAAGCCCATCCTGGAGAAGATGGACGGCACCGAGG |
| | | AGCTGCTGGTGAAGCTGAACCGGGAGGACCTGCTGCGGAAGCAGCGGACCTTCGACAACGGCTCCATCCCCCACCAGATCCACCTGGG |
| | | CGAGCTGCACGCCATCCTGCGGCGGCAGGAGGACTTCTACCCCTTCCTGAAGGACAACCGGGAGAAGATCGAGAAGATCCTGACCTTC |
| | | CGGATCCCCTACTACGTGGGCCCCCTGGCCCGGGGCAACTCCCGGTTCGCCTGGATGACCCGGAAGTCCGAGGAGACCATCACCCCCT |
| | | GGAACTTCGAGGAGGTGGTGGACAAGGGCGCCTCCGCCCAGTCCTTCATCGAGCGGATGACCAACTTCGACAAGAACCTGCCCAACGA |
| | | GAAGGTGCTGCCCAAGCACTCCCTGCTGTACGAGTACTTCACCGTGTACAACGAGCTGACCAAGGTGAAGTACGTGACCGAGGGCATG |
| | | CGGAAGCCCGCCTTCCTGTCCGGCGAGCAGAAGAAGGCCATCGTGGACCTGCTGTTCAAGACCAACCGGAAGGTGACCGTGAAGCAGC |
| | | TGAAGGAGGACTACTTCAAGAAGATCGAGTGCTTCGACTCCGTGGAGATCTCCGGCGTGGAGGACCGGTTCAACGCCTCCCTGGGCAC |
| | | CTACCACGACCTGCTGAAGATCATCAAGGACAAGGACTTCCTGGACAACGAGGAGAACGAGGACATCCTGGAGGACATCGTGCTGACC |
| | | CTGACCCTGTTCGAGGACCGGGAGATGATCGAGGAGCGGCTGAAGACCTACGCCCACCTGTTCGACGACAAGGTGATGAAGCAGCTGA |
| | | AGCGGCGGCGGTACACCGGCTGGGGCCGGCTGTCCCGGAAGCTGATCAACGGCATCCGGGACAAGCAGTCCGGCAAGACCATCCTGGA |
| | | CTTCCTGAAGTCCGACGGCTTCGCCAACCGGAACTTCATGCAGCTGATCCACGACGACTCCCTGACCTTCAAGGAGGACATCCAGAAG |
| | | GCCCAGGTGTCCGGCCAGGGCGACTCCCTGCACGAGCACATCGCCAACCTGGCCGGCTCCCCCGCCATCAAGAAGGGCATCCTGCAGA |
| | | CCGTGAAGGTGGTGGACGAGCTGGTGAAGGTGATGGGCCGGCACAAGCCCGAGAACATCGTGATCGAGATGGCCCGGGAGAACCAGAC |
| | | CACCCAGAAGGGCCAGAAGAACTCCCGGGAGCGGATGAAGCGGATCGAGGAGGGCATCAAGGAGCTGGGCTCCCAGATCCTGAAGGAG |
| | | CACCCCGTGGAGAACACCCAGCTGCAGAACGAGAAGCTGTACCTGTACTACCTGCAGAACGGCCGGGACATGTACGTGGACCAGGAGC |
| | | TGGACATCAACCGGCTGTCCGACTACGACGTGGACCACATCGTGCCCCAGTCCTTCCTGAAGGACGACTCCATCGACAACAAGGTGCT |
| | | GACCCGGTCCGACAAGAACCGGGGCAAGTCCGACAACGTGCCCTCCGAGGAGGTGGTGAAGAAGATGAAGAACTACTGGCGGCAGCTG |
| | | CTGAACGCCAAGCTGATCACCCAGCGGAAGTTCGACAACCTGACCAAGGCCGAGCGGGGCGGCCTGTCCGAGCTGGACAAGGCCGGCT |
| | | TCATCAAGCGGCAGCTGGTGGAGACCCGGCAGATCACCAAGCACGTGGCCCAGATCCTGGACTCCCGGATGAACACCAAGTACGACGA |
| | | GAACGACAAGCTGATCCGGGAGGTGAAGGTGATCACCCTGAAGTCCAAGCTGGTGTCCGACTTCCGGAAGGACTTCCAGTTCTACAAG |
| | | GTGCGGGAGATCAACAACTACCACCACGCCCACGACGCCTACCTGAACGCCGTGGTGGGCACCGCCCTGATCAAGAAGTACCCCAAGC |
| | | TGGAGTCCGAGTTCGTGTACGGCGACTACAAGGTGTACGACGTGCGGAAGATGATCGCCAAGTCCGAGCAGGAGATCGGCAAGGCCAC |
| | | CGCCAAGTACTTCTTCTACTCCAACATCATGAACTTCTTCAAGACCGAGATCACCCTGGCCAACGGCGAGATCCGGAAGCGGCCCCTG |
| | | ATCGAGACCAACGGCGAGACCGGCGAGATCGTGTGGGACAAGGGCCGGGACTTCGCCACCGTGCGGAAGGTGCTGTCCATGCCCCAGG |
| | | TGAACATCGTGAAGAAGACCGAGGTGCAGACCGGCGGCTTCTCCAAGGAGTCCATCCTGCCCAAGCGGAACTCCGACAAGCTGATCGC |
| | | CCGGAAGAAGGACTGGGACCCCAAGAAGTACGGCGGCTTCGACTCCCCCACCGTGGCCTACTCCGTGCTGGTGGTGGCCAAGGTGGAG |
| | | AAGGGCAAGTCCAAGAAGCTGAAGTCCGTGAAGGAGCTGCTGGGCATCACCATCATGGAGCGGTCCTCCTTCGAGAAGAACCCCATCG |
| | | ACTTCCTGGAGGCCAAGGGCTACAAGGAGGTGAAGAAGGACCTGATCATCAAGCTGCCCAAGTACTCCCTGTTCGAGCTGGAGAACGG |
| | | CCGGAAGCGGATGCTGGCCTCCGCCGGCGAGCTGCAGAAGGGCAACGAGCTGGCCCTGCCCTCCAAGTACGTGAACTTCCTGTACCTG |
| | | GCCTCCCACTACGAGAAGCTGAAGGGCTCCCCCGAGGACAACGAGCAGAAGCAGCTGTTCGTGGAGCAGCACAAGCACTACCTGGACG |
| | | AGATCATCGAGCAGATCTCCGAGTTCTCCAAGCGGGTGATCCTGGCCGACGCCAACCTGGACAAGGTGCTGTCCGCCTACAACAAGCA |
| | | CCGGGACAAGCCCATCCGGGAGCAGGCCGAGAACATCATCCACCTGTTCACCCTGACCAACCTGGGCGCCCCCGCCGCCTTCAAGTAC |
| | | TTCGACACCACCATCGACCGGAAGCGGTACACCTCCACCAAGGAGGTGCTGGACGCCACCCTGATCCACCAGTCCATCACCGGCCTGT |
| | | ACGAGACCCGGATCGACCTGTCCCAGCTGGGCGGCGACGGCGGCGGCTCCCCCAAGAAGAAGCGGAAGGTGTCCGAGTCCGCCACCCC |
| | | CGAGTCCGTGTCCGGCTGGCGGCTGTTCAAGAAGATCTCCTGACTAGCACCAGCCTCAAGAACACCCGAATGGAGTCTCTAAGCTACA |
| | | TAATACCAACTTACACTTTACAAAATGTTGTCCCCCAAAATGTAGCCATTCGTATCTGCTCCTAATAAAAAGAAAGTTTCTTCACATT |
| | | CTCTCGAGAAAAAAAAAAAATGGAAAAAAAAAAAACGGAAAAAAAAAAAAGGTAAAAAAAAAAAATATAAAAAAAAAAAACATAAAAA |
| | | AAAAAAACGAAAAAAAAAAAACGTAAAAAAAAAAAACTCAAAAAAAAAAAAGATAAAAAAAAAAAACCTAAAAAAAAAAAATGTAAAA |
| | | AAAAAAAAGGGAAAAAAAAAAAACGCAAAAAAAAAAAACACAAAAAAAAAAAATGCAAAAAAAAAAAATCGAAAAAAAAAAAATCTAA |
| | | AAAAAAAAAACGAAAAAAAAAAAACCCAAAAAAAAAAAAGACAAAAAAAAAAAATAGAAAAAAAAAAAAGTTAAAAAAAAAAAACTGA |
| | | AAAAAAAAAAATTTAAAAAAAAAAAATCTAG |
| 823 | ORF encoding UGI | ATGACCAACCTGTCCGACATCATCGAGAAGGAGACCGGCAAGCAGCTGGTGATCCAGGAGTCCATCCTGATGCTGCCCGAGGAGGTGG |
| | | AGGAGGTGATCGGCAACAAGCCCGAGTCCGACATCCTGGTGCACACCGCCTACGACGAGTCCACCGACGAGAACGTGATGCTGCTGAC |
| | | CTCCGACGCCCCCGAGTACAAGCCCTGGGCCCTGGTGATCCAGGACTCCAACGGCGAGAACAAGATCAAGATGCTGTCCGGCGGCTCC |
| | | AAGCGGACCGCCGACGGCTCCGAGTTCGAGTCCCCCAAGAAGAAGCGGAAGGTGGAGTGA |
| 824 | ORF encoding UGI | ATGGGACCGAAGAAGAAGAGAAAGGTCGGAGGAGGAAGCACAAACCTGTCGGACATCATCGAAAAGGAAACAGGAAAGCAGCTGGTCA |
| | | TCCAGGAATCGATCCTGATGCTGCCGGAAGAAGTCGAAGAAGTCATCGGAAACAAGCCGGAATCGGACATCCTGGTCCACACAGCATA |
| | | CGACGAATCGACAGACGAAAACGTCATGCTGCTGACATCGGACGCACCGGAATACAAGCCGTGGGCACTGGTCATCCAGGACTCGAAC |
| | | GGAGAAAACAAGATCAAGATGCTGTGA |
| 825 | ORF encoding UGI | ATGACCAACCTGTCCGACATCATCGAGAAGGAGACCGGCAAGCAGCTGGTGATCCAGGAGTCCATCCTGATGCTGCCCGAGGAGGTGG |
| | with Hibit tag | AGGAGGTGATCGGCAACAAGCCCGAGTCCGACATCCTGGTGCACACCGCCTACGACGAGTCCACCGACGAGAACGTGATGCTGCTGAC |
| | | CTCCGACGCCCCCGAGTACAAGCCCTGGGCCCTGGTGATCCAGGACTCCAACGGCGAGAACAAGATCAAGATGCTGTCCGGCGGCTCC |
| | | AAGCGGACCGCCGACGGCTCCGAGTTCGAGTCCCCCAAGAAGAAGCGGAAGGTGGAGTCCGAGTCCGCCACCCCCGAGTCCGTGTCCG |
| | | GCTGGCGGCTGTTCAAGAAGATCTCCTGA |
| 826 | ORF encoding UGI | ATGACCAACCTGTCCGACATCATCGAGAAGGAGACCGGCAAGCAGCTGGTGATCCAGGAGTCCATCCTGATGCTGCCCGAGGAGGTGG |
| | | AGGAGGTGATCGGCAACAAGCCCGAGTCCGACATCCTGGTGCACACCGCCTACGACGAGTCCACCGACGAGAACGTGATGCTGCTGAC |
| | | CTCCGACGCCCCCGAGTACAAGCCCTGGGCCCTGGTGATCCAGGACTCCAACGGCGAGAACAAGATCAAGATGCTGTCCGGCGGCTCC |
| | | AAGCGGACCGCCGACGGCTCCGAGTTCGAGTCCCCCAAGAAGAAGCGGAAGGTGGAGTGATAG |
| 827 | Exemplary ORF encoding D10A SpyCas9 | ATGGACAAGAAGTACTCCATCGGCCTGGCCATCGGCACCAACTCCGTGGGCTGGGCCGTGATCACCGACGAGTACAAGGTGCCCTCCA |
| | nickase with 1x NLS as the C-terminal | AGAAGTTCAAGGTGCTGGGCAACACCGACCGGCACTCCATCAAGAAGAACCTGATCGGCGCCCTGCTGTTCGACTCCGGCGAGACCGC |
| | amino acids | CGAGGCCACCCGGCTGAAGCGGACCGCCCGGCGGCGGTACACCCGGCGGAAGAACCGGATCTGCTACCTGCAGGAGATCTTCTCCAAC |
| | | GAGATGGCCAAGGTGGACGACTCCTTCTTCCACCGGCTGGAGGAGTCCTTCCTGGTGGAGGAGGACAAGAAGCACGAGCGGCACCCCA |
| | | TCTTCGGCAACATCGTGGACGAGGTGGCCTACCACGAGAAGTACCCCACCATCTACCACCTGCGGAAGAAGCTGGTGGACTCCACCGA |
| | | CAAGGCCGACCTGCGGCTGATCTACCTGGCCCTGGCCCACATGATCAAGTTCCGGGGCCACTTCCTGATCGAGGGCGACCTGAACCCC |
| | | GACAACTCCGACGTGGACAAGCTGTTCATCCAGCTGGTGCAGACCTACAACCAGCTGTTCGAGGAGAACCCCATCAACGCCTCCGGCG |
| | | TGGACGCCAAGGCCATCCTGTCCGCCCGGCTGTCCAAGTCCCGGCGGCTGGAGAACCTGATCGCCCAGCTGCCCGGCGAGAAGAAGAA |
| | | CGGCCTGTTCGGCAACCTGATCGCCCTGTCCCTGGGCCTGACCCCCAACTTCAAGTCCAACTTCGACCTGGCCGAGGACGCCAAGCTG |
| | | CAGCTGTCCAAGGACACCTACGACGACGACCTGGACAACCTGCTGGCCCAGATCGGCGACCAGTACGCCGACCTGTTCCTGGCCGCCA |
| | | AGAACCTGTCCGACGCCATCCTGCTGTCCGACATCCTGCGGGTGAACACCGAGATCACCAAGGCCCCCCTGTCCGCCTCCATGATCAA |
| | | GCGGTACGACGAGCACCACCAGGACCTGACCCTGCTGAAGGCCCTGGTGCGGCAGCAGCTGCCCGAGAAGTACAAGGAGATCTTCTTC |
| | | GACCAGTCCAAGAACGGCTACGCCGGCTACATCGACGGCGGCGCCTCCCAGGAGGAGTTCTACAAGTTCATCAAGCCCATCCTGGAGA |
| | | AGATGGACGGCACCGAGGAGCTGCTGGTGAAGCTGAACCGGGAGGACCTGCTGCGGAAGCAGCGGACCTTCGACAACGGCTCCATCCC |
| | | CCACCAGATCCACCTGGGCGAGCTGCACGCCATCCTGCGGCGGCAGGAGGACTTCTACCCCTTCCTGAAGGACAACCGGGAGAAGATC |
| | | GAGAAGATCCTGACCTTCCGGATCCCCTACTACGTGGGCCCCCTGGCCCGGGGCAACTCCCGGTTCGCCTGGATGACCCGGAAGTCCG |
| | | AGGAGACCATCACCCCCTGGAACTTCGAGGAGGTGGTGGACAAGGGCGCCTCCGCCCAGTCCTTCATCGAGCGGATGACCAACTTCGA |
| | | CAAGAACCTGCCCAACGAGAAGGTGCTGCCCAAGCACTCCCTGCTGTACGAGTACTTCACCGTGTACAACGAGCTGACCAAGGTGAAG |
| | | TACGTGACCGAGGGCATGCGGAAGCCCGCCTTCCTGTCCGGCGAGCAGAAGAAGGCCATCGTGGACCTGCTGTTCAAGACCAACCGGA |
| | | AGGTGACCGTGAAGCAGCTGAAGGAGGACTACTTCAAGAAGATCGAGTGCTTCGACTCCGTGGAGATCTCCGGCGTGGAGGACCGGTT |
| | | CAACGCCTCCCTGGGCACCTACCACGACCTGCTGAAGATCATCAAGGACAAGGACTTCCTGGACAACGAGGAGAACGAGGACATCCTG |
| | | GAGGACATCGTGCTGACCCTGACCCTGTTCGAGGACCGGGAGATGATCGAGGAGCGGCTGAAGACCTACGCCCACCTGTTCGACGACA |
| | | AGGTGATGAAGCAGCTGAAGCGGCGGCGGTACACCGGCTGGGGCCGGCTGTCCCGGAAGCTGATCAACGGCATCCGGGACAAGCAGTC |
| | | CGGCAAGACCATCCTGGACTTCCTGAAGTCCGACGGCTTCGCCAACCGGAACTTCATGCAGCTGATCCACGACGACTCCCTGACCTTC |
| | | AAGGAGGACATCCAGAAGGCCCAGGTGTCCGGCCAGGGCGACTCCCTGCACGAGCACATCGCCAACCTGGCCGGCTCCCCCGCCATCA |
| | | AGAAGGGCATCCTGCAGACCGTGAAGGTGGTGGACGAGCTGGTGAAGGTGATGGGCCGGCACAAGCCCGAGAACATCGTGATCGAGAT |
| | | GGCCCGGGAGAACCAGACCACCCAGAAGGGCCAGAAGAACTCCCGGGAGCGGATGAAGCGGATCGAGGAGGGCATCAAGGAGCTGGGC |
| | | TCCCAGATCCTGAAGGAGCACCCCGTGGAGAACACCCAGCTGCAGAACGAGAAGCTGTACCTGTACTACCTGCAGAACGGCCGGGACA |
| | | TGTACGTGGACCAGGAGCTGGACATCAACCGGCTGTCCGACTACGACGTGGACCACATCGTGCCCCAGTCCTTCCTGAAGGACGACTC |
| | | CATCGACAACAAGGTGCTGACCCGGTCCGACAAGAACCGGGGCAAGTCCGACAACGTGCCCTCCGAGGAGGTGGTGAAGAAGATGAAG |
| | | AACTACTGGCGGCAGCTGCTGAACGCCAAGCTGATCACCCAGCGGAAGTTCGACAACCTGACCAAGGCCGAGCGGGGCGGCCTGTCCG |
| | | AGCTGGACAAGGCCGGCTTCATCAAGCGGCAGCTGGTGGAGACCCGGCAGATCACCAAGCACGTGGCCCAGATCCTGGACTCCCGGAT |
| | | GAACACCAAGTACGACGAGAACGACAAGCTGATCCGGGAGGTGAAGGTGATCACCCTGAAGTCCAAGCTGGTGTCCGACTTCCGGAAG |
| | | GACTTCCAGTTCTACAAGGTGCGGGAGATCAACAACTACCACCACGCCCACGACGCCTACCTGAACGCCGTGGTGGGCACCGCCCTGA |
| | | TCAAGAAGTACCCCAAGCTGGAGTCCGAGTTCGTGTACGGCGACTACAAGGTGTACGACGTGCGGAAGATGATCGCCAAGTCCGAGCA |
| | | GGAGATCGGCAAGGCCACCGCCAAGTACTTCTTCTACTCCAACATCATGAACTTCTTCAAGACCGAGATCACCCTGGCCAACGGCGAG |
| | | ATCCGGAAGCGGCCCCTGATCGAGACCAACGGCGAGACCGGCGAGATCGTGTGGGACAAGGGCCGGGACTTCGCCACCGTGCGGAAGG |
| | | TGCTGTCCATGCCCCAGGTGAACATCGTGAAGAAGACCGAGGTGCAGACCGGCGGCTTCTCCAAGGAGTCCATCCTGCCCAAGCGGAA |
| | | CTCCGACAAGCTGATCGCCCGGAAGAAGGACTGGGACCCCAAGAAGTACGGCGGCTTCGACTCCCCCACCGTGGCCTACTCCGTGCTG |
| | | GTGGTGGCCAAGGTGGAGAAGGGCAAGTCCAAGAAGCTGAAGTCCGTGAAGGAGCTGCTGGGCATCACCATCATGGAGCGGTCCTCCT |
| | | TCGAGAAGAACCCCATCGACTTCCTGGAGGCCAAGGGCTACAAGGAGGTGAAGAAGGACCTGATCATCAAGCTGCCCAAGTACTCCCT |
| | | GTTCGAGCTGGAGAACGGCCGGAAGCGGATGCTGGCCTCCGCCGGCGAGCTGCAGAAGGGCAACGAGCTGGCCCTGCCCTCCAAGTAC |
| | | GTGAACTTCCTGTACCTGGCCTCCCACTACGAGAAGCTGAAGGGCTCCCCCGAGGACAACGAGCAGAAGCAGCTGTTCGTGGAGCAGC |
| | | ACAAGCACTACCTGGACGAGATCATCGAGCAGATCTCCGAGTTCTCCAAGCGGGTGATCCTGGCCGACGCCAACCTGGACAAGGTGCT |
| | | GTCCGCCTACAACAAGCACCGGGACAAGCCCATCCGGGAGCAGGCCGAGAACATCATCCACCTGTTCACCCTGACCAACCTGGGCGCC |
| | | CCCGCCGCCTTCAAGTACTTCGACACCACCATCGACCGGAAGCGGTACACCTCCACCAAGGAGGTGCTGGACGCCACCCTGATCCACC |
| | | AGTCCATCACCGGCCTGTACGAGACCCGGATCGACCTGTCCCAGCTGGGCGGCGACGGCGGCGGCTCCCCCAAGAAGAAGCGGAAGGT |
| | | GTGA |
| 828 | Exemplary ORF encoding D10A SpyCas9 | ATGGACAAGAAGTACTCCATCGGCCTGGCCATCGGCACCAACTCCGTGGGCTGGGCCGTGATCACCGACGAGTACAAGGTGCCCTCCA |
| | nickase with no NLS | AGAAGTTCAAGGTGCTGGGCAACACCGACCGGCACTCCATCAAGAAGAACCTGATCGGCGCCCTGCTGTTCGACTCCGGCGAGACCGC |
| | | CGAGGCCACCCGGCTGAAGCGGACCGCCCGGCGGCGGTACACCCGGCGGAAGAACCGGATCTGCTACCTGCAGGAGATCTTCTCCAAC |
| | | GAGATGGCCAAGGTGGACGACTCCTTCTTCCACCGGCTGGAGGAGTCCTTCCTGGTGGAGGAGGACAAGAAGCACGAGCGGCACCCCA |
| | | TCTTCGGCAACATCGTGGACGAGGTGGCCTACCACGAGAAGTACCCCACCATCTACCACCTGCGGAAGAAGCTGGTGGACTCCACCGA |
| | | CAAGGCCGACCTGCGGCTGATCTACCTGGCCCTGGCCCACATGATCAAGTTCCGGGGCCACTTCCTGATCGAGGGCGACCTGAACCCC |
| | | GACAACTCCGACGTGGACAAGCTGTTCATCCAGCTGGTGCAGACCTACAACCAGCTGTTCGAGGAGAACCCCATCAACGCCTCCGGCG |
| | | TGGACGCCAAGGCCATCCTGTCCGCCCGGCTGTCCAAGTCCCGGCGGCTGGAGAACCTGATCGCCCAGCTGCCCGGCGAGAAGAAGAA |
| | | CGGCCTGTTCGGCAACCTGATCGCCCTGTCCCTGGGCCTGACCCCCAACTTCAAGTCCAACTTCGACCTGGCCGAGGACGCCAAGCTG |
| | | CAGCTGTCCAAGGACACCTACGACGACGACCTGGACAACCTGCTGGCCCAGATCGGCGACCAGTACGCCGACCTGTTCCTGGCCGCCA |
| | | AGAACCTGTCCGACGCCATCCTGCTGTCCGACATCCTGCGGGTGAACACCGAGATCACCAAGGCCCCCCTGTCCGCCTCCATGATCAA |
| | | GCGGTACGACGAGCACCACCAGGACCTGACCCTGCTGAAGGCCCTGGTGCGGCAGCAGCTGCCCGAGAAGTACAAGGAGATCTTCTTC |
| | | GACCAGTCCAAGAACGGCTACGCCGGCTACATCGACGGCGGCGCCTCCCAGGAGGAGTTCTACAAGTTCATCAAGCCCATCCTGGAGA |
| | | AGATGGACGGCACCGAGGAGCTGCTGGTGAAGCTGAACCGGGAGGACCTGCTGCGGAAGCAGCGGACCTTCGACAACGGCTCCATCCC |
| | | CCACCAGATCCACCTGGGCGAGCTGCACGCCATCCTGCGGCGGCAGGAGGACTTCTACCCCTTCCTGAAGGACAACCGGGAGAAGATC |
| | | GAGAAGATCCTGACCTTCCGGATCCCCTACTACGTGGGCCCCCTGGCCCGGGGCAACTCCCGGTTCGCCTGGATGACCCGGAAGTCCG |
| | | AGGAGACCATCACCCCCTGGAACTTCGAGGAGGTGGTGGACAAGGGCGCCTCCGCCCAGTCCTTCATCGAGCGGATGACCAACTTCGA |
| | | CAAGAACCTGCCCAACGAGAAGGTGCTGCCCAAGCACTCCCTGCTGTACGAGTACTTCACCGTGTACAACGAGCTGACCAAGGTGAAG |
| | | TACGTGACCGAGGGCATGCGGAAGCCCGCCTTCCTGTCCGGCGAGCAGAAGAAGGCCATCGTGGACCTGCTGTTCAAGACCAACCGGA |
| | | AGGTGACCGTGAAGCAGCTGAAGGAGGACTACTTCAAGAAGATCGAGTGCTTCGACTCCGTGGAGATCTCCGGCGTGGAGGACCGGTT |
| | | CAACGCCTCCCTGGGCACCTACCACGACCTGCTGAAGATCATCAAGGACAAGGACTTCCTGGACAACGAGGAGAACGAGGACATCCTG |
| | | GAGGACATCGTGCTGACCCTGACCCTGTTCGAGGACCGGGAGATGATCGAGGAGCGGCTGAAGACCTACGCCCACCTGTTCGACGACA |
| | | AGGTGATGAAGCAGCTGAAGCGGCGGCGGTACACCGGCTGGGGCCGGCTGTCCCGGAAGCTGATCAACGGCATCCGGGACAAGCAGTC |
| | | CGGCAAGACCATCCTGGACTTCCTGAAGTCCGACGGCTTCGCCAACCGGAACTTCATGCAGCTGATCCACGACGACTCCCTGACCTTC |
| | | AAGGAGGACATCCAGAAGGCCCAGGTGTCCGGCCAGGGCGACTCCCTGCACGAGCACATCGCCAACCTGGCCGGCTCCCCCGCCATCA |
| | | AGAAGGGCATCCTGCAGACCGTGAAGGTGGTGGACGAGCTGGTGAAGGTGATGGGCCGGCACAAGCCCGAGAACATCGTGATCGAGAT |
| | | GGCCCGGGAGAACCAGACCACCCAGAAGGGCCAGAAGAACTCCCGGGAGCGGATGAAGCGGATCGAGGAGGGCATCAAGGAGCTGGGC |
| | | TCCCAGATCCTGAAGGAGCACCCCGTGGAGAACACCCAGCTGCAGAACGAGAAGCTGTACCTGTACTACCTGCAGAACGGCCGGGACA |
| | | TGTACGTGGACCAGGAGCTGGACATCAACCGGCTGTCCGACTACGACGTGGACCACATCGTGCCCCAGTCCTTCCTGAAGGACGACTC |
| | | CATCGACAACAAGGTGCTGACCCGGTCCGACAAGAACCGGGGCAAGTCCGACAACGTGCCCTCCGAGGAGGTGGTGAAGAAGATGAAG |
| | | AACTACTGGCGGCAGCTGCTGAACGCCAAGCTGATCACCCAGCGGAAGTTCGACAACCTGACCAAGGCCGAGCGGGGCGGCCTGTCCG |
| | | AGCTGGACAAGGCCGGCTTCATCAAGCGGCAGCTGGTGGAGACCCGGCAGATCACCAAGCACGTGGCCCAGATCCTGGACTCCCGGAT |
| | | GAACACCAAGTACGACGAGAACGACAAGCTGATCCGGGAGGTGAAGGTGATCACCCTGAAGTCCAAGCTGGTGTCCGACTTCCGGAAG |
| | | GACTTCCAGTTCTACAAGGTGCGGGAGATCAACAACTACCACCACGCCCACGACGCCTACCTGAACGCCGTGGTGGGCACCGCCCTGA |
| | | TCAAGAAGTACCCCAAGCTGGAGTCCGAGTTCGTGTACGGCGACTACAAGGTGTACGACGTGCGGAAGATGATCGCCAAGTCCGAGCA |
| | | GGAGATCGGCAAGGCCACCGCCAAGTACTTCTTCTACTCCAACATCATGAACTTCTTCAAGACCGAGATCACCCTGGCCAACGGCGAG |
| | | ATCCGGAAGCGGCCCCTGATCGAGACCAACGGCGAGACCGGCGAGATCGTGTGGGACAAGGGCCGGGACTTCGCCACCGTGCGGAAGG |
| | | TGCTGTCCATGCCCCAGGTGAACATCGTGAAGAAGACCGAGGTGCAGACCGGCGGCTTCTCCAAGGAGTCCATCCTGCCCAAGCGGAA |
| | | CTCCGACAAGCTGATCGCCCGGAAGAAGGACTGGGACCCCAAGAAGTACGGCGGCTTCGACTCCCCCACCGTGGCCTACTCCGTGCTG |
| | | GTGGTGGCCAAGGTGGAGAAGGGCAAGTCCAAGAAGCTGAAGTCCGTGAAGGAGCTGCTGGGCATCACCATCATGGAGCGGTCCTCCT |
| | | TCGAGAAGAACCCCATCGACTTCCTGGAGGCCAAGGGCTACAAGGAGGTGAAGAAGGACCTGATCATCAAGCTGCCCAAGTACTCCCT |
| | | GTTCGAGCTGGAGAACGGCCGGAAGCGGATGCTGGCCTCCGCCGGCGAGCTGCAGAAGGGCAACGAGCTGGCCCTGCCCTCCAAGTAC |
| | | GTGAACTTCCTGTACCTGGCCTCCCACTACGAGAAGCTGAAGGGCTCCCCCGAGGACAACGAGCAGAAGCAGCTGTTCGTGGAGCAGC |
| | | ACAAGCACTACCTGGACGAGATCATCGAGCAGATCTCCGAGTTCTCCAAGCGGGTGATCCTGGCCGACGCCAACCTGGACAAGGTGCT |
| | | GTCCGCCTACAACAAGCACCGGGACAAGCCCATCCGGGAGCAGGCCGAGAACATCATCCACCTGTTCACCCTGACCAACCTGGGCGCC |
| | | CCCGCCGCCTTCAAGTACTTCGACACCACCATCGACCGGAAGCGGTACACCTCCACCAAGGAGGTGCTGGACGCCACCCTGATCCACC |
| | | AGTCCATCACCGGCCTGTACGAGACCCGGATCGACCTGTCCCAGCTGGGCGGCGACTGA |
| 831 | Exemplary AA sequence for Nme2Cas9 base | MDGSGGGSPKKKRKVEDKRPAATKKAGQAKKKKGGSGGGEASPASGPRHLMDPHIFTSNFNNGIGRHKTYLCYEVERLDNGTSVKMDQ |
| | editor | HRGFLHNQAKNLLCGFYGRHAELRFLDLVPSLQLDPAQIYRVTWFISWSPCFSWGCAGEVRAFLQENTHVRLRIFAARIYDYDPLYKE |
| | | ALQMLRDAGAQVSIMTYDEFKHCWDTFVDHQGCPFQPWDGLDEHSQALSGRLRAILQNQGNSGSETPGTSESATPESAAFKPNPINYI |
| | | LGLAIGIASVGWAMVEIDEEENPIRLIDLGVRVFERAEVPKTGDSLAMARRLARSVRRLTRRRAHRLLRARRLLKREGVLQAADFDEN |
| | | GLIKSLPNTPWQLRAAALDRKLTPLEWSAVLLHLIKHRGYLSQRKNEGETADKELGALLKGVANNAHALQTGDFRTPAELALNKFEKE |
| | | SGHIRNQRGDYSHTFSRKDLQAELILLFEKQKEFGNPHVSGGLKEGIETLLMTQRPALSGDAVQKMLGHCTFEPAEPKAAKNTYTAER |
| | | FIWLTKLNNLRILEQGSERPLTDTERATLMDEPYRKSKLTYAQARKLLGLEDTAFFKGLRYGKDNAEASTLMEMKAYHAISRALEKEG |
| | | LKDKKSPLNLSSELQDEIGTAFSLFKTDEDITGRLKDRVQPEILEALLKHISFDKFVQISLKALRRIVPLMEQGKRYDEACAEIYGDH |
| | | YGKKNTEEKIYLPPIPADEIRNPVVLRALSQARKVINGVVRRYGSPARIHIETAREVGKSFKDRKEIEKRQEENRKDREKAAAKFREY |
| | | FPNFVGEPKSKDILKLRLYEQQHGKCLYSGKEINLVRLNEKGYVEIDHALPFSRTWDDSFNNKVLVLGSENQNKGNQTPYEYFNGKDN |
| | | SREWQEFKARVETSRFPRSKKQRILLQKFDEDGFKECNLNDTRYVNRFLCQFVADHILLTGKGKRRVFASNGQITNLLRGFWGLRKVR |
| | | AENDRHHALDAVVVACSTVAMQQKITRFVRYKEMNAFDGKTIDKETGKVLHQKTHFPQPWEFFAQEVMIRVFGKPDGKPEFEEADTPE |
| | | KLRTLLAEKLSSRPEAVHEYVTPLFVSRAPNRKMSGAHKDTLRSAKRFVKHNEKISVKRVWLTEIKLADLENMVNYKNGREIELYEAL |
| | | KARLEAYGGNAKQAFDPKDNPFYKKGGQLVKAVRVEKTQESGVLLNKKNAYTIADNGDMVRVDVFCKVDKKGKNQYFIVPIYAWQVAE |
| | | NILPDIDCKGYRIDDSYTFCFSLHKYDLIAFQKDEKSKVEFAYYINCDSSNGRFYLAWHDKGSKEQQFRISTQNLVLIQKYQVNELGK |
| | | EIRPCRLKKRPPVR |
| 832 | Exemplary AA sequence of Nme2Cas9 cleavase | MAAFKPNPINYILGLDIGIASVGWAMVEIDEEENPIRLIDLGVRVFERAEVPKTGDSLAMARRLARSVRRLTRRRAHRLLRARRLLKR |
| | | EGVLQAADFDENGLIKSLPNTPWQLRAAALDRKLTPLEWSAVLLHLIKHRGYLSQRKNEGETADKELGALLKGVANNAHALQTGDFRT |
| | | PAELALNKFEKESGHIRNQRGDYSHTFSRKDLQAELILLFEKQKEFGNPHVSGGLKEGIETLLMTQRPALSGDAVQKMLGHCTFEPAE |
| | | PKAAKNTYTAERFIWLTKLNNLRILEQGSERPLTDTERATLMDEPYRKSKLTYAQARKLLGLEDTAFFKGLRYGKDNAEASTLMEMKA |
| | | YHAISRALEKEGLKDKKSPLNLSSELQDEIGTAFSLFKTDEDITGRLKDRVQPEILEALLKHISFDKFVQISLKALRRIVPLMEQGKR |
| | | YDEACAEIYGDHYGKKNTEEKIYLPPIPADEIRNPVVLRALSQARKVINGVVRRYGSPARIHIETAREVGKSFKDRKEIEKRQEENRK |
| | | DREKAAAKFREYFPNFVGEPKSKDILKLRLYEQQHGKCLYSGKEINLVRLNEKGYVEIDHALPFSRTWDDSFNNKVLVLGSENQNKGN |
| | | QTPYEYFNGKDNSREWQEFKARVETSRFPRSKKQRILLQKFDEDGFKECNLNDTRYVNRFLCQFVADHILLTGKGKRRVFASNGQITN |
| | | LLRGFWGLRKVRAENDRHHALDAVVVACSTVAMQQKITRFVRYKEMNAFDGKTIDKETGKVLHQKTHFPQPWEFFAQEVMIRVFGKPD |
| | | GKPEFEEADTPEKLRTLLAEKLSSRPEAVHEYVTPLFVSRAPNRKMSGAHKDTLRSAKRFVKHNEKISVKRVWLTEIKLADLENMVNY |
| | | KNGREIELYEALKARLEAYGGNAKQAFDPKDNPFYKKGGQLVKAVRVEKTQESGVLLNKKNAYTIADNGDMVRVDVFCKVDKKGKNQY |
| | | FIVPIYAWQVAENILPDIDCKGYRIDDSYTFCFSLHKYDLIAFQKDEKSKVEFAYYINCDSSNGRFYLAWHDKGSKEQQFRISTQNLV |
| | | LIQKYQVNELGKEIRPCRLKKRPPVR |
| 833 | Exemplary AA sequence of Nme2Cas9 cleavase | MDGSGGGSPKKKRKVEDKRPAATKKAGQAKKKKGGSGGGAAFKPNPINYILGLDIGIASVGWAMVEIDEEENPIRLIDLGVRVFERAE |
| | HiBit | VPKTGDSLAMARRLARSVRRLTRRRAHRLLRARRLLKREGVLQAADFDENGLIKSLPNTPWQLRAAALDRKLIPLEWSAVLLHLIKHR |
| | | GYLSQRKNEGETADKELGALLKGVANNAHALQTGDFRTPAELALNKFEKESGHIRNQRGDYSHTFSRKDLQAELILLFEKQKEFGNPH |
| | | VSGGLKEGIETLLMTQRPALSGDAVQKMLGHCTFEPAEPKAAKNTYTAERFIWLTKLNNLRILEQGSERPLTDTERATLMDEPYRKSK |
| | | LTYAQARKLLGLEDTAFFKGLRYGKDNAEASTLMEMKAYHAISRALEKEGLKDKKSPLNLSSELQDEIGTAFSLFKTDEDITGRLKDR |
| | | VQPEILEALLKHISFDKFVQISLKALRRIVPLMEQGKRYDEACAEIYGDHYGKKNTEEKIYLPPIPADEIRNPVVLRALSQARKVING |
| | | VVRRYGSPARIHIETAREVGKSFKDRKEIEKRQEENRKDREKAAAKFREYFPNFVGEPKSKDILKLRLYEQQHGKCLYSGKEINLVRL |
| | | NEKGYVEIDHALPFSRTWDDSFNNKVLVLGSENQNKGNQTPYEYFNGKDNSREWQEFKARVETSRFPRSKKQRILLQKFDEDGEKECN |
| | | LNDTRYVNRFLCQFVADHILLTGKGKRRVFASNGQITNLLRGFWGLRKVRAENDRHHALDAVVVACSTVAMQQKITRFVRYKEMNAFD |
| | | GKTIDKETGKVLHQKTHFPQPWEFFAQEVMIRVFGKPDGKPEFEEADTPEKLRTLLAEKLSSRPEAVHEYVTPLFVSRAPNRKMSGAH |
| | | KDTLRSAKRFVKHNEKISVKRVWLTEIKLADLENMVNYKNGREIELYEALKARLEAYGGNAKQAFDPKDNPFYKKGGQLVKAVRVEKT |
| | | QESGVLLNKKNAYTIADNGDMVRVDVFCKVDKKGKNQYFIVPIYAWQVAENILPDIDCKGYRIDDSYTFCFSLHKYDLIAFQKDEKSK |
| | | VEFAYYINCDSSNGRFYLAWHDKGSKEQQFRISTQNLVLIQKYQVNELGKEIRPCRLKKRPPVRSESATPESVSGWRLFKKIS |
| 834 | Exemplary AA sequence for D16A Nme2Cas9 | MAAFKPNPINYILGLAIGIASVGWAMVEIDEEENPIRLIDLGVRVFERAEVPKTGDSLAMARRLARSVRRLTRRRAHRLLRARRLLKR |
| | nickase | EGVLQAADFDENGLIKSLPNTPWQLRAAALDRKLTPLEWSAVLLHLIKHRGYLSQRKNEGETADKELGALLKGVANNAHALQTGDFRT |
| | | PAELALNKFEKESGHIRNQRGDYSHTFSRKDLQAELILLFEKQKEFGNPHVSGGLKEGIETLLMTQRPALSGDAVQKMLGHCTFEPAE |
| | | PKAAKNTYTAERFIWLTKLNNLRILEQGSERPLTDTERATLMDEPYRKSKLTYAQARKLLGLEDTAFFKGLRYGKDNAEASTLMEMKA |
| | | YHAISRALEKEGLKDKKSPLNLSSELQDEIGTAFSLFKTDEDITGRLKDRVQPEILEALLKHISFDKFVQISLKALRRIVPLMEQGKR |
| | | YDEACAEIYGDHYGKKNTEEKIYLPPIPADEIRNPVVLRALSQARKVINGVVRRYGSPARIHIETAREVGKSFKDRKEIEKRQEENRK |
| | | DREKAAAKFREYFPNFVGEPKSKDILKLRLYEQQHGKCLYSGKEINLVRLNEKGYVEIDHALPFSRTWDDSFNNKVLVLGSENQNKGN |
| | | QTPYEYFNGKDNSREWQEFKARVETSRFPRSKKQRILLQKFDEDGFKECNLNDTRYVNRFLCQFVADHILLTGKGKRRVFASNGQITN |
| | | LLRGFWGLRKVRAENDRHHALDAVVVACSTVAMQQKITRFVRYKEMNAFDGKTIDKETGKVLHQKTHFPQPWEFFAQEVMIRVFGKPD |
| | | GKPEFEEADTPEKLRTLLAEKLSSRPEAVHEYVTPLFVSRAPNRKMSGAHKDTLRSAKRFVKHNEKISVKRVWLTEIKLADLENMVNY |
| | | KNGREIELYEALKARLEAYGGNAKQAFDPKDNPFYKKGGQLVKAVRVEKTQESGVLLNKKNAYTIADNGDMVRVDVFCKVDKKGKNQY |
| | | FIVPIYAWQVAENILPDIDCKGYRIDDSYTFCFSLHKYDLIAFQKDEKSKVEFAYYINCDSSNGRFYLAWHDKGSKEQQFRISTQNLV |
| | | LIQKYQVNELGKEIRPCRLKKRPPVR |
| 835 | Exemplary AA sequence of Nme2 base editor | MDGSGGGSPKKKRKVEDKRPAATKKAGQAKKKKGGSGGGEASPASGPRHLMDPHIFTSNFNNGIGRHKTYLCYEVERLDNGTSVKMDQ |
| | (NLS-NLS-APOBEC3A-L070-Nme2D16A) | HRGFLHNQAKNLLCGFYGRHAELRFLDLVPSLQLDPAQIYRVTWFISWSPCFSWGCAGEVRAFLQENTHVRLRIFAARIYDYDPLYKE |
| | | ALQMLRDAGAQVSIMTYDEFKHCWDTFVDHQGCPFQPWDGLDEHSQALSGRLRAILQNQGNGTKDSTKDIPETPSKDAAFKPNPINYI |
| | | LGLAIGIASVGWAMVEIDEEENPIRLIDLGVRVFERAEVPKTGDSLAMARRLARSVRRLTRRRAHRLLRARRLLKREGVLQAADFDEN |
| | | GLIKSLPNTPWQLRAAALDRKLTPLEWSAVLLHLIKHRGYLSQRKNEGETADKELGALLKGVANNAHALQTGDFRTPAELALNKFEKE |
| | | SGHIRNQRGDYSHTFSRKDLQAELILLFEKQKEFGNPHVSGGLKEGIETLLMTQRPALSGDAVQKMLGHCTFEPAEPKAAKNTYTAER |
| | | FIWLTKLNNLRILEQGSERPLTDTERATLMDEPYRKSKLTYAQARKLLGLEDTAFFKGLRYGKDNAEASTLMEMKAYHAISRALEKEG |
| | | LKDKKSPLNLSSELQDEIGTAFSLFKTDEDITGRLKDRVQPEILEALLKHISFDKFVQISLKALRRIVPLMEQGKRYDEACAEIYGDH |
| | | YGKKNTEEKIYLPPIPADEIRNPVVLRALSQARKVINGVVRRYGSPARIHIETAREVGKSFKDRKEIEKRQEENRKDREKAAAKFREY |
| | | FPNFVGEPKSKDILKLRLYEQQHGKCLYSGKEINLVRLNEKGYVEIDHALPFSRTWDDSFNNKVLVLGSENQNKGNQTPYEYFNGKDN |
| | | SREWQEFKARVETSRFPRSKKQRILLQKFDEDGFKECNLNDTRYVNRFLCQFVADHILLTGKGKRRVFASNGQITNLLRGFWGLRKVR |
| | | AENDRHHALDAVVVACSTVAMQQKITRFVRYKEMNAFDGKTIDKETGKVLHQKTHFPQPWEFFAQEVMIRVFGKPDGKPEFEEADTPE |
| | | KLRTLLAEKLSSRPEAVHEYVTPLFVSRAPNRKMSGAHKDTLRSAKRFVKHNEKISVKRVWLTEIKLADLENMVNYKNGREIELYEAL |
| | | KARLEAYGGNAKQAFDPKDNPFYKKGGQLVKAVRVEKTQESGVLLNKKNAYTIADNGDMVRVDVFCKVDKKGKNQYFIVPIYAWQVAE |
| | | NILPDIDCKGYRIDDSYTFCFSLHKYDLIAFQKDEKSKVEFAYYINCDSSNGRFYLAWHDKGSKEQQFRISTQNLVLIQKYQVNELGK |
| | | EIRPCRLKKRPPVR |
| 836 | Exemplary AA sequence of APOBEC3A-Nme2D16A | MEASPASGPRHLMDPHIFTSNFNNGIGRHKTYLCYEVERLDNGTSVKMDQHRGFLHNQAKNLLCGFYGRHAELRFLDLVPSLQLDPAQ |
| | | IYRVTWFISWSPCFSWGCAGEVRAFLQENTHVRLRIFAARIYDYDPLYKEALQMLRDAGAQVSIMTYDEFKHCWDTFVDHQGCPFQPW |
| | | DGLDEHSQALSGRLRAILQNQGNSGSETPGTSESATPESAAFKPNPINYILGLAIGIASVGWAMVEIDEEENPIRLIDLGVRVFERAE |
| | | VPKTGDSLAMARRLARSVRRLTRRRAHRLLRARRLLKREGVLQAADFDENGLIKSLPNTPWQLRAAALDRKLTPLEWSAVLLHLIKHR |
| | | GYLSQRKNEGETADKELGALLKGVANNAHALQTGDFRTPAELALNKFEKESGHIRNQRGDYSHTFSRKDLQAELILLFEKQKEFGNPH |
| | | VSGGLKEGIETLLMTQRPALSGDAVQKMLGHCTFEPAEPKAAKNTYTAERFIWLTKLNNLRILEQGSERPLTDTERATLMDEPYRKSK |
| | | LTYAQARKLLGLEDTAFFKGLRYGKDNAEASTLMEMKAYHAISRALEKEGLKDKKSPLNLSSELQDEIGTAFSLFKTDEDITGRLKDR |
| | | VQPEILEALLKHISFDKFVQISLKALRRIVPLMEQGKRYDEACAEIYGDHYGKKNTEEKIYLPPIPADEIRNPVVLRALSQARKVING |
| | | VVRRYGSPARIHIETAREVGKSFKDRKEIEKRQEENRKDREKAAAKFREYFPNFVGEPKSKDILKLRLYEQQHGKCLYSGKEINLVRL |
| | | NEKGYVEIDHALPFSRTWDDSFNNKVLVLGSENQNKGNQTPYEYFNGKDNSREWQEFKARVETSRFPRSKKQRILLQKFDEDGFKECN |
| | | LNDTRYVNRFLCQFVADHILLTGKGKRRVFASNGQITNLLRGFWGLRKVRAENDRHHALDAVVVACSTVAMQQKITRFVRYKEMNAFD |
| | | GKTIDKETGKVLHQKTHFPQPWEFFAQEVMIRVFGKPDGKPEFEEADTPEKLRTLLAEKLSSRPEAVHEYVTPLFVSRAPNRKMSGAH |
| | | KDTLRSAKRFVKHNEKISVKRVWLTEIKLADLENMVNYKNGREIELYEALKARLEAYGGNAKQAFDPKDNPFYKKGGQLVKAVRVEKT |
| | | QESGVLLNKKNAYTIADNGDMVRVDVFCKVDKKGKNQYFIVPIYAWQVAENILPDIDCKGYRIDDSYTFCFSLHKYDLIAFQKDEKSK |
| | | VEFAYYINCDSSNGRFYLAWHDKGSKEQQFRISTQNLVLIQKYQVNELGKEIRPCRLKKRPPVRSGKRTADGSEFESPKKKRKVE |
| 837 | Exemplary AA sequence of APOBEC3A-Nme2D16A | MDGSGGGSPKKKRKVEDKRPAATKKAGQAKKKKGGSGGGEASPASGPRHLMDPHIFTSNFNNGIGRHKTYLCYEVERLDNGTSVKMDQ |
| | | HRGFLHNQAKNLLCGFYGRHAELRFLDLVPSLQLDPAQIYRVTWFISWSPCFSWGCAGEVRAFLQENTHVRLRIFAARIYDYDPLYKE |
| | | ALQMLRDAGAQVSIMTYDEFKHCWDTFVDHQGCPFQPWDGLDEHSQALSGRLRAILQNQGNSGSETPGTSESATPESAAFKPNPINYI |
| | | LGLAIGIASVGWAMVEIDEEENPIRLIDLGVRVFERAEVPKTGDSLAMARRLARSVRRLTRRRAHRLLRARRLLKREGVLQAADFDEN |
| | | GLIKSLPNTPWQLRAAALDRKLTPLEWSAVLLHLIKHRGYLSQRKNEGETADKELGALLKGVANNAHALQTGDFRTPAELALNKFEKE |
| | | SGHIRNQRGDYSHTFSRKDLQAELILLFEKQKEFGNPHVSGGLKEGIETLLMTQRPALSGDAVQKMLGHCTFEPAEPKAAKNTYTAER |
| | | FIWLTKLNNLRILEQGSERPLTDTERATLMDEPYRKSKLTYAQARKLLGLEDTAFFKGLRYGKDNAEASTLMEMKAYHAISRALEKEG |
| | | LKDKKSPLNLSSELQDEIGTAFSLFKTDEDITGRLKDRVQPEILEALLKHISFDKFVQISLKALRRIVPLMEQGKRYDEACAEIYGDH |
| | | YGKKNTEEKIYLPPIPADEIRNPVVLRALSQARKVINGVVRRYGSPARIHIETAREVGKSFKDRKEIEKRQEENRKDREKAAAKFREY |
| | | FPNFVGEPKSKDILKLRLYEQQHGKCLYSGKEINLVRLNEKGYVEIDHALPFSRTWDDSFNNKVLVLGSENQNKGNQTPYEYENGKDN |
| | | SREWQEFKARVETSRFPRSKKQRILLQKFDEDGFKECNLNDTRYVNRFLCQFVADHILLTGKGKRRVFASNGQITNLLRGFWGLRKVR |
| | | AENDRHHALDAVVVACSTVAMQQKITRFVRYKEMNAFDGKTIDKETGKVLHQKTHFPQPWEFFAQEVMIRVFGKPDGKPEFEEADTPE |
| | | KLRTLLAEKLSSRPEAVHEYVTPLFVSRAPNRKMSGAHKDTLRSAKRFVKHNEKISVKRVWLTEIKLADLENMVNYKNGREIELYEAL |
| | | KARLEAYGGNAKQAFDPKDNPFYKKGGQLVKAVRVEKTQESGVLLNKKNAYTIADNGDMVRVDVFCKVDKKGKNQYFIVPIYAWQVAE |
| | | NILPDIDCKGYRIDDSYTFCFSLHKYDLIAFQKDEKSKVEFAYYINCDSSNGRFYLAWHDKGSKEQQFRISTQNLVLIQKYQVNELGK |
| | | EIRPCRLKKRPPVRSGKRTADGSEFESPKKKRKVE |
| 838 | Exemplary AA sequence of APOBEC3A-Nme2D16A | MDGSGGGSPKKKRKVEDKRPAATKKAGQAKKKKGGSGGGEASPASGPRHLMDPHIFTSNFNNGIGRHKTYLCYEVERLDNGTSVKMDQ |
| | | HRGFLHNQAKNLLCGFYGRHAELRFLDLVPSLQLDPAQIYRVTWFISWSPCFSWGCAGEVRAFLQENTHVRLRIFAARIYDYDPLYKE |
| | | ALQMLRDAGAQVSIMTYDEFKHCWDTFVDHQGCPFQPWDGLDEHSQALSGRLRAILQNQGNSGSETPGTSESATPESAAFKPNPINYI |
| | | LGLAIGIASVGWAMVEIDEEENPIRLIDLGVRVFERAEVPKTGDSLAMARRLARSVRRLTRRRAHRLLRARRLLKREGVLQAADFDEN |
| | | GLIKSLPNTPWQLRAAALDRKLTPLEWSAVLLHLIKHRGYLSQRKNEGETADKELGALLKGVANNAHALQTGDFRTPAELALNKFEKE |
| | | SGHIRNQRGDYSHTFSRKDLQAELILLFEKQKEFGNPHVSGGLKEGIETLLMTQRPALSGDAVQKMLGHCTFEPAEPKAAKNTYTAER |
| | | FIWLTKLNNLRILEQGSERPLTDTERATLMDEPYRKSKLTYAQARKLLGLEDTAFFKGLRYGKDNAEASTLMEMKAYHAISRALEKEG |
| | | LKDKKSPLNLSSELQDEIGTAFSLFKTDEDITGRLKDRVQPEILEALLKHISFDKFVQISLKALRRIVPLMEQGKRYDEACAEIYGDH |
| | | YGKKNTEEKIYLPPIPADEIRNPVVLRALSQARKVINGVVRRYGSPARIHIETAREVGKSFKDRKEIEKRQEENRKDREKAAAKFREY |
| | | FPNFVGEPKSKDILKLRLYEQQHGKCLYSGKEINLVRLNEKGYVEIDHALPFSRTWDDSFNNKVLVLGSENQNKGNQTPYEYENGKDN |
| | | SREWQEFKARVETSRFPRSKKQRILLQKFDEDGFKECNLNDTRYVNRFLCQFVADHILLTGKGKRRVFASNGQITNLLRGFWGLRKVR |
| | | AENDRHHALDAVVVACSTVAMQQKITRFVRYKEMNAFDGKTIDKETGKVLHQKTHFPQPWEFFAQEVMIRVFGKPDGKPEFEEADTPE |
| | | KLRTLLAEKLSSRPEAVHEYVTPLFVSRAPNRKMSGAHKDTLRSAKRFVKHNEKISVKRVWLTEIKLADLENMVNYKNGREIELYEAL |
| | | KARLEAYGGNAKQAFDPKDNPFYKKGGQLVKAVRVEKTQESGVLLNKKNAYTIADNGDMVRVDVFCKVDKKGKNQYFIVPIYAWQVAE |
| | | NILPDIDCKGYRIDDSYTFCFSLHKYDLIAFQKDEKSKVEFAYYINCDSSNGRFYLAWHDKGSKEQQFRISTQNLVLIQKYQVNELGK |
| | | EIRPCRLKKRPPVREDKRPAATKKAGQAKKKKYPYDVPDYAGYPYDVPDYAGSYPYDVPDYAAAPAAKKKKLD |
| | 839-849 | Not used |
| 850 | Exemplary AA sequence of H. sapiens | MEASPASGPRHLMDPHIFTSNFNNGIGRHKTYLCYEVERLDNGTSVKMDQHRGFLHNQAKNLLCGFYGRHAELRFLDLVPSLQLDPAQ |
| | APOBEC3A deaminase (A3A), see BC22 | IYRVTWFISWSPCFSWGCAGEVRAFLQENTHVRLRIFAARIYDYDPLYKEALQMLRDAGAQVSIMTYDEFKHCWDTFVDHQGCPFQPW |
| | | DGLDEHSQALSGRLRAILQNQGN |
| 851 | Exemplary AA sequence for SpyCas9 base | MEASPASGPRHLMDPHIFTSNFNNGIGRHKTYLCYEVERLDNGTSVKMDQHRGFLHNQAKNLLCGFYGRHAELRFLDLVPSLQLDPAQ |
| | editor | IYRVTWFISWSPCFSWGCAGEVRAFLQENTHVRLRIFAARIYDYDPLYKEALQMLRDAGAQVSIMTYDEFKHCWDTFVDHQGCPFQPW |
| | | DGLDEHSQALSGRLRAILQNQGNSGSETPGTSESATPESDKKYSIGLAIGTNSVGWAVITDEYKVPSKKFKVLGNTDRHSIKKNLIGA |
| | | LLFDSGETAEATRLKRTARRRYTRRKNRICYLQEIFSNEMAKVDDSFFHRLEESFLVEEDKKHERHPIFGNIVDEVAYHEKYPTIYHL |
| | | RKKLVDSTDKADLRLIYLALAHMIKFRGHFLIEGDLNPDNSDVDKLFIQLVQTYNQLFEENPINASGVDAKAILSARLSKSRRLENLI |
| | | AQLPGEKKNGLFGNLIALSLGLTPNFKSNFDLAEDAKLQLSKDTYDDDLDNLLAQIGDQYADLFLAAKNLSDAILLSDILRVNTEITK |
| | | APLSASMIKRYDEHHQDLTLLKALVRQQLPEKYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSRFAWMTRKSEETITPWNFEEVVDKGASAQS |
| | | FIERMTNFDKNLPNEKVLPKHSLLYEYFTVYNELTKVKYVTEGMRKPAFLSGEQKKAIVDLLFKTNRKVTVKQLKEDYFKKIECFDSV |
| | | EISGVEDRFNASLGTYHDLLKIIKDKDFLDNEENEDILEDIVLTLTLFEDREMIEERLKTYAHLFDDKVMKQLKRRRYTGWGRLSRKL |
| | | INGIRDKQSGKTILDFLKSDGFANRNFMQLIHDDSLTFKEDIQKAQVSGQGDSLHEHIANLAGSPAIKKGILQTVKVVDELVKVMGRH |
| | | KPENIVIEMARENQTTQKGQKNSRERMKRIEEGIKELGSQILKEHPVENTQLQNEKLYLYYLQNGRDMYVDQELDINRLSDYDVDHIV |
| | | PQSFLKDDSIDNKVLTRSDKNRGKSDNVPSEEVVKKMKNYWRQLLNAKLITQRKFDNLTKAERGGLSELDKAGFIKRQLVETRQITKH |
| | | VAQILDSRMNTKYDENDKLIREVKVITLKSKLVSDFRKDFQFYKVREINNYHHAHDAYLNAVVGTALIKKYPKLESEFVYGDYKVYDV |
| | | RKMIAKSEQEIGKATAKYFFYSNIMNFFKTEITLANGEIRKRPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKGKSKKLKSVKELLGITIMERSSFEKNPIDFLEAKGYKEVKKDL |
| | | IIKLPKYSLFELENGRKRMLASAGELQKGNELALPSKYVNFLYLASHYEKLKGSPEDNEQKQLFVEQHKHYLDEIIEQISEFSKRVIL |
| | | ADANLDKVLSAYNKHRDKPIREQAENIIHLFTLTNLGAPAAFKYFDTTIDRKRYTSTKEVLDATLIHQSITGLYETRIDLSQLGGDGG |
| | | GSPKKKRKV |
| 852 | Exemplary AA sequence for SpyCas9 base | MEASPASGPRHLMDPHIFTSNFNNGIGRHKTYLCYEVERLDNGTSVKMDQHRGFLHNQAKNLLCGFYGRHAELRFLDLVPSLQLDPAQ |
| | editor with Hibit tag | IYRVTWFISWSPCFSWGCAGEVRAFLQENTHVRLRIFAARIYDYDPLYKEALQMLRDAGAQVSIMTYDEFKHCWDTFVDHQGCPFQPW |
| | | DGLDEHSQALSGRLRAILQNQGNSGSETPGTSESATPESDKKYSIGLAIGTNSVGWAVITDEYKVPSKKFKVLGNTDRHSIKKNLIGA |
| | | LLFDSGETAEATRLKRTARRRYTRRKNRICYLQEIFSNEMAKVDDSFFHRLEESFLVEEDKKHERHPIFGNIVDEVAYHEKYPTIYHL |
| | | RKKLVDSTDKADLRLIYLALAHMIKFRGHFLIEGDLNPDNSDVDKLFIQLVQTYNQLFEENPINASGVDAKAILSARLSKSRRLENLI |
| | | AQLPGEKKNGLFGNLIALSLGLTPNFKSNFDLAEDAKLQLSKDTYDDDLDNLLAQIGDQYADLFLAAKNLSDAILLSDILRVNTEITK |
| | | APLSASMIKRYDEHHQDLTLLKALVRQQLPEKYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSRFAWMTRKSEETITPWNFEEVVDKGASAQS |
| | | FIERMTNFDKNLPNEKVLPKHSLLYEYFTVYNELTKVKYVTEGMRKPAFLSGEQKKAIVDLLFKTNRKVTVKQLKEDYFKKIECFDSV |
| | | EISGVEDRFNASLGTYHDLLKIIKDKDFLDNEENEDILEDIVLTLTLFEDREMIEERLKTYAHLFDDKVMKQLKRRRYTGWGRLSRKL |
| | | INGIRDKQSGKTILDFLKSDGFANRNFMQLIHDDSLTFKEDIQKAQVSGQGDSLHEHIANLAGSPAIKKGILQTVKVVDELVKVMGRH |
| | | KPENIVIEMARENQTTQKGQKNSRERMKRIEEGIKELGSQILKEHPVENTQLQNEKLYLYYLQNGRDMYVDQELDINRLSDYDVDHIV |
| | | PQSFLKDDSIDNKVLTRSDKNRGKSDNVPSEEVVKKMKNYWRQLLNAKLITQRKFDNLTKAERGGLSELDKAGFIKRQLVETRQITKH |
| | | VAQILDSRMNTKYDENDKLIREVKVITLKSKLVSDFRKDFQFYKVREINNYHHAHDAYLNAVVGTALIKKYPKLESEFVYGDYKVYDV |
| | | RKMIAKSEQEIGKATAKYFFYSNIMNFFKTEITLANGEIRKRPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKGKSKKLKSVKELLGITIMERSSFEKNPIDFLEAKGYKEVKKDL |
| | | IIKLPKYSLFELENGRKRMLASAGELQKGNELALPSKYVNFLYLASHYEKLKGSPEDNEQKQLFVEQHKHYLDEIIEQISEFSKRVIL |
| | | ADANLDKVLSAYNKHRDKPIREQAENIIHLFTLTNLGAPAAFKYFDTTIDRKRYTSTKEVLDATLIHQSITGLYETRIDLSQLGGDGG |
| | | GSPKKKRKVSESATPESVSGWRLFKKIS |
| 853 | Exemplary AA sequence for SpyCas9 | MDKKYSIGLDIGTNSVGWAVITDEYKVPSKKFKVLGNTDRHSIKKNLIGALLFDSGETAEATRLKRTARRRYTRRKNRICYLQEIFSN |
| | | EMAKVDDSFFHRLEESFLVEEDKKHERHPIFGNIVDEVAYHEKYPTIYHLRKKLVDSTDKADLRLIYLALAHMIKFRGHFLIEGDLNP |
| | | DNSDVDKLFIQLVQTYNQLFEENPINASGVDAKAILSARLSKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTEITKAPLSASMIKRYDEHHQDLTLLKALVRQQLPEKYKEIFF |
| | | DQSKNGYAGYIDGGASQEEFYKFIKPILEKMDGTEELLVKLNREDLLRKQRTFDNGSIPHQIHLGELHAILRRQEDFYPFLKDNREKI |
| | | EKILTFRIPYYVGPLARGNSRFAWMTRKSEETITPWNFEEVVDKGASAQSFIERMTNFDKNLPNEKVLPKHSLLYEYFTVYNELTKVK |
| | | YVTEGMRKPAFLSGEQKKAIVDLLFKTNRKVTVKQLKEDYFKKIECFDSVEISGVEDRFNASLGTYHDLLKIIKDKDELDNEENEDIL |
| | | EDIVLTLTLFEDREMIEERLKTYAHLFDDKVMKQLKRRRYTGWGRLSRKLINGIRDKQSGKTILDFLKSDGFANRNFMQLIHDDSLTF |
| | | KEDIQKAQVSGQGDSLHEHIANLAGSPAIKKGILQTVKVVDELVKVMGRHKPENIVIEMARENQTTQKGQKNSRERMKRIEEGIKELG |
| | | SQILKEHPVENTQLQNEKLYLYYLQNGRDMYVDQELDINRLSDYDVDHIVPQSFLKDDSIDNKVLTRSDKNRGKSDNVPSEEVVKKMK |
| | | NYWRQLLNAKLITQRKFDNLTKAERGGLSELDKAGFIKRQLVETRQITKHVAQILDSRMNTKYDENDKLIREVKVITLKSKLVSDERK |
| | | DFQFYKVREINNYHHAHDAYLNAVVGTALIKKYPKLESEFVYGDYKVYDVRKMIAKSEQEIGKATAKYFFYSNIMNFFKTEITLANGE |
| | | IRKRPLIETNGETGEIVWDKGRDFATVRKVLSMPQVNIVKKTEVQTGGFSKESILPKRNSDKLIARKKDWDPKKYGGFDSPTVAYSVL |
| | | VVAKVEKGKSKKLKSVKELLGITIMERSSFEKNPIDFLEAKGYKEVKKDLIIKLPKYSLFELENGRKRMLASAGELQKGNELALPSKY |
| | | VNFLYLASHYEKLKGSPEDNEQKQLFVEQHKHYLDEIIEQISEFSKRVILADANLDKVLSAYNKHRDKPIREQAENIIHLFTLTNLGA |
| | | PAAFKYFDTTIDRKRYTSTKEVLDATLIHQSITGLYETRIDLSQLGGDGGGSPKKKRKV |
| 854 | Exemplary AA sequence for SpyCas9 with | MDKKYSIGLDIGTNSVGWAVITDEYKVPSKKFKVLGNTDRHSIKKNLIGALLFDSGETAEATRLKRTARRRYTRRKNRICYLQEIFSN |
| | Hibit tab | EMAKVDDSFFHRLEESFLVEEDKKHERHPIFGNIVDEVAYHEKYPTIYHLRKKLVDSTDKADLRLIYLALAHMIKFRGHFLIEGDLNP |
| | | DNSDVDKLFIQLVQTYNQLFEENPINASGVDAKAILSARLSKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTEITKAPLSASMIKRYDEHHQDLTLLKALVRQQLPEKYKEIFF |
| | | DQSKNGYAGYIDGGASQEEFYKFIKPILEKMDGTEELLVKLNREDLLRKQRTFDNGSIPHQIHLGELHAILRRQEDFYPFLKDNREKI |
| | | EKILTFRIPYYVGPLARGNSRFAWMTRKSEETITPWNFEEVVDKGASAQSFIERMTNFDKNLPNEKVLPKHSLLYEYFTVYNELTKVK |
| | | YVTEGMRKPAFLSGEQKKAIVDLLFKTNRKVTVKQLKEDYFKKIECFDSVEISGVEDRFNASLGTYHDLLKIIKDKDFLDNEENEDIL |
| | | EDIVLTLTLFEDREMIEERLKTYAHLFDDKVMKQLKRRRYTGWGRLSRKLINGIRDKQSGKTILDFLKSDGFANRNFMQLIHDDSLTF |
| | | KEDIQKAQVSGQGDSLHEHIANLAGSPAIKKGILQTVKVVDELVKVMGRHKPENIVIEMARENQTTQKGQKNSRERMKRIEEGIKELG |
| | | SQILKEHPVENTQLQNEKLYLYYLQNGRDMYVDQELDINRLSDYDVDHIVPQSFLKDDSIDNKVLTRSDKNRGKSDNVPSEEVVKKMK |
| | | NYWRQLLNAKLITQRKFDNLTKAERGGLSELDKAGFIKRQLVETRQITKHVAQILDSRMNTKYDENDKLIREVKVITLKSKLVSDERK |
| | | DFQFYKVREINNYHHAHDAYLNAVVGTALIKKYPKLESEFVYGDYKVYDVRKMIAKSEQEIGKATAKYFFYSNIMNFFKTEITLANGE |
| | | IRKRPLIETNGETGEIVWDKGRDFATVRKVLSMPQVNIVKKTEVQTGGFSKESILPKRNSDKLIARKKDWDPKKYGGFDSPTVAYSVL |
| | | VVAKVEKGKSKKLKSVKELLGITIMERSSFEKNPIDFLEAKGYKEVKKDLIIKLPKYSLFELENGRKRMLASAGELQKGNELALPSKY |
| | | VNFLYLASHYEKLKGSPEDNEQKQLFVEQHKHYLDEIIEQISEFSKRVILADANLDKVLSAYNKHRDKPIREQAENIIHLFTLINLGA |
| | | PAAFKYFDTTIDRKRYTSTKEVLDATLIHQSITGLYETRIDLSQLGGDGGGSPKKKRKVSESATPESVSGWRLFKKIS |
| 855 | Exemplary AA sequence of D10A SpyCas9 | MDKKYSIGLAIGTNSVGWAVITDEYKVPSKKFKVLGNTDRHSIKKNLIGALLFDSGETAEATRLKRTARRRYTRRKNRICYLQEIFSN |
| | nickase with 1x NLS as the C-terminal 7 | EMAKVDDSFFHRLEESFLVEEDKKHERHPIFGNIVDEVAYHEKYPTIYHLRKKLVDSTDKADLRLIYLALAHMIKFRGHFLIEGDLNP |
| | Exemplary AAs | DNSDVDKLFIQLVQTYNQLFEENPINASGVDAKAILSARLSKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTEITKAPLSASMIKRYDEHHQDLTLLKALVRQQLPEKYKEIFF |
| | | DQSKNGYAGYIDGGASQEEFYKFIKPILEKMDGTEELLVKLNREDLLRKQRTFDNGSIPHQIHLGELHAILRRQEDFYPFLKDNREKI |
| | | EKILTFRIPYYVGPLARGNSRFAWMTRKSEETITPWNFEEVVDKGASAQSFIERMTNFDKNLPNEKVLPKHSLLYEYFTVYNELTKVK |
| | | YVTEGMRKPAFLSGEQKKAIVDLLFKTNRKVTVKQLKEDYFKKIECFDSVEISGVEDRFNASLGTYHDLLKIIKDKDFLDNEENEDIL |
| | | EDIVLTLTLFEDREMIEERLKTYAHLFDDKVMKQLKRRRYTGWGRLSRKLINGIRDKQSGKTILDFLKSDGFANRNFMQLIHDDSLTF |
| | | KEDIQKAQVSGQGDSLHEHIANLAGSPAIKKGILQTVKVVDELVKVMGRHKPENIVIEMARENQTTQKGQKNSRERMKRIEEGIKELG |
| | | SQILKEHPVENTQLQNEKLYLYYLQNGRDMYVDQELDINRLSDYDVDHIVPQSFLKDDSIDNKVLTRSDKNRGKSDNVPSEEVVKKMK |
| | | NYWRQLLNAKLITQRKFDNLTKAERGGLSELDKAGFIKRQLVETRQITKHVAQILDSRMNTKYDENDKLIREVKVITLKSKLVSDFRK |
| | | DFQFYKVREINNYHHAHDAYLNAVVGTALIKKYPKLESEFVYGDYKVYDVRKMIAKSEQEIGKATAKYFFYSNIMNFFKTEITLANGE |
| | | IRKRPLIETNGETGEIVWDKGRDFATVRKVLSMPQVNIVKKTEVQTGGFSKESILPKRNSDKLIARKKDWDPKKYGGFDSPTVAYSVL |
| | | VVAKVEKGKSKKLKSVKELLGITIMERSSFEKNPIDFLEAKGYKEVKKDLIIKLPKYSLFELENGRKRMLASAGELQKGNELALPSKY |
| | | VNFLYLASHYEKLKGSPEDNEQKQLFVEQHKHYLDEIIEQISEFSKRVILADANLDKVLSAYNKHRDKPIREQAENIIHLFTLTNLGA |
| | | PAAFKYFDTTIDRKRYTSTKEVLDATLIHQSITGLYETRIDLSQLGGDGGGSPKKKRKV |
| 856 | Exemplary AA sequence of Cas9 nickase | MDKKYSIGLAIGTNSVGWAVITDEYKVPSKKFKVLGNTDRHSIKKNLIGALLFDSGETAEATRLKRTARRRYTRRKNRICYLQEIFSN |
| | (without NLS) | EMAKVDDSFFHRLEESFLVEEDKKHERHPIFGNIVDEVAYHEKYPTIYHLRKKLVDSTDKADLRLIYLALAHMIKFRGHFLIEGDLNP |
| | | DNSDVDKLFIQLVQTYNQLFEENPINASGVDAKAILSARLSKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTEITKAPLSASMIKRYDEHHQDLTLLKALVRQQLPEKYKEIFF |
| | | DQSKNGYAGYIDGGASQEEFYKFIKPILEKMDGTEELLVKLNREDLLRKQRTFDNGSIPHQIHLGELHAILRRQEDFYPFLKDNREKI |
| | | EKILTFRIPYYVGPLARGNSRFAWMTRKSEETITPWNFEEVVDKGASAQSFIERMTNFDKNLPNEKVLPKHSLLYEYFTVYNELTKVK |
| | | YVTEGMRKPAFLSGEQKKAIVDLLFKTNRKVTVKQLKEDYFKKIECFDSVEISGVEDRFNASLGTYHDLLKIIKDKDELDNEENEDIL |
| | | EDIVLTLTLFEDREMIEERLKTYAHLFDDKVMKQLKRRRYTGWGRLSRKLINGIRDKQSGKTILDFLKSDGFANRNFMQLIHDDSLTF |
| | | KEDIQKAQVSGQGDSLHEHIANLAGSPAIKKGILQTVKVVDELVKVMGRHKPENIVIEMARENQTTQKGQKNSRERMKRIEEGIKELG |
| | | SQILKEHPVENTQLQNEKLYLYYLQNGRDMYVDQELDINRLSDYDVDHIVPQSFLKDDSIDNKVLTRSDKNRGKSDNVPSEEVVKKMK |
| | | NYWRQLLNAKLITQRKFDNLTKAERGGLSELDKAGFIKRQLVETRQITKHVAQILDSRMNTKYDENDKLIREVKVITLKSKLVSDFRK |
| | | DFQFYKVREINNYHHAHDAYLNAVVGTALIKKYPKLESEFVYGDYKVYDVRKMIAKSEQEIGKATAKYFFYSNIMNFFKTEITLANGE |
| | | IRKRPLIETNGETGEIVWDKGRDFATVRKVLSMPQVNIVKKTEVQTGGFSKESILPKRNSDKLIARKKDWDPKKYGGFDSPTVAYSVL |
| | | VVAKVEKGKSKKLKSVKELLGITIMERSSFEKNPIDFLEAKGYKEVKKDLIIKLPKYSLFELENGRKRMLASAGELQKGNELALPSKY |
| | | VNFLYLASHYEKLKGSPEDNEQKQLFVEQHKHYLDEIIEQISEFSKRVILADANLDKVLSAYNKHRDKPIREQAENIIHLFTLINLGA |
| | | PAAFKYFDTTIDRKRYTSTKEVLDATLIHQSITGLYETRIDLSQLGGD |
| 857 | Exemplary AA sequence of D10A SpyCas9 | DKKYSIGLAIGTNSVGWAVITDEYKVPSKKFKVLGNTDRHSIKKNLIGALLFDSGETAEATRLKRTARRRYTRRKNRICYLQEIFSNE |
| | nickase with two NLS as the C-terminal | MAKVDDSFFHRLEESFLVEEDKKHERHPIFGNIVDEVAYHEKYPTIYHLRKKLVDSTDKADLRLIYLALAHMIKERGHFLIEGDLNPD |
| | Exemplary AAs | NSDVDKLFIQLVQTYNQLFEENPINASGVDAKAILSARLSKSRRLENLIAQLPGEKKNGLFGNLIALSLGLTPNFKSNFDLAEDAKLQ |
| | | LSKDTYDDDLDNLLAQIGDQYADLFLAAKNLSDAILLSDILRVNTEITKAPLSASMIKRYDEHHQDLTLLKALVRQQLPEKYKEIFFD |
| | | QSKNGYAGYIDGGASQEEFYKFIKPILEKMDGTEELLVKLNREDLLRKQRTFDNGSIPHQIHLGELHAILRRQEDFYPFLKDNREKIE |
| | | KILTFRIPYYVGPLARGNSRFAWMTRKSEETITPWNFEEVVDKGASAQSFIERMTNFDKNLPNEKVLPKHSLLYEYFTVYNELTKVKY |
| | | VTEGMRKPAFLSGEQKKAIVDLLFKTNRKVTVKQLKEDYFKKIECFDSVEISGVEDRFNASLGTYHDLLKIIKDKDFLDNEENEDILE |
| | | DIVLTLTLFEDREMIEERLKTYAHLFDDKVMKQLKRRRYTGWGRLSRKLINGIRDKQSGKTILDFLKSDGFANRNFMQLIHDDSLTFK |
| | | EDIQKAQVSGQGDSLHEHIANLAGSPAIKKGILQTVKVVDELVKVMGRHKPENIVIEMARENQTTQKGQKNSRERMKRIEEGIKELGS |
| | | QILKEHPVENTQLQNEKLYLYYLQNGRDMYVDQELDINRLSDYDVDHIVPQSFLKDDSIDNKVLTRSDKNRGKSDNVPSEEVVKKMKN |
| | | YWRQLLNAKLITQRKFDNLTKAERGGLSELDKAGFIKRQLVETRQITKHVAQILDSRMNTKYDENDKLIREVKVITLKSKLVSDFRKD |
| | | FQFYKVREINNYHHAHDAYLNAVVGTALIKKYPKLESEFVYGDYKVYDVRKMIAKSEQEIGKATAKYFFYSNIMNFFKTEITLANGEI |
| | | RKRPLIETNGETGEIVWDKGRDFATVRKVLSMPQVNIVKKTEVQTGGFSKESILPKRNSDKLIARKKDWDPKKYGGFDSPTVAYSVLV |
| | | VAKVEKGKSKKLKSVKELLGITIMERSSFEKNPIDFLEAKGYKEVKKDLIIKLPKYSLFELENGRKRMLASAGELQKGNELALPSKYV |
| | | NFLYLASHYEKLKGSPEDNEQKQLFVEQHKHYLDEIIEQISEFSKRVILADANLDKVLSAYNKHRDKPIREQAENIIHLFTLTNLGAP |
| | | AAFKYFDTTIDRKRYTSTKEVLDATLIHQSITGLYETRIDLSQLGGDGSGSPKKKRKVDGSPKKKRKVDSG |
| 858 | Exemplary AA sequence for base editor with | MEASPASGPRHLMDPHIFTSNFNNGIGRHKTYLCYEVERLDNGTSVKMDQHRGFLHNQAKNLLCGFYGRHAELRFLDLVPSLQLDPAQ |
| | linker L070 | IYRVTWFISWSPCFSWGCAGEVRAFLQENTHVRLRIFAARIYDYDPLYKEALQMLRDAGAQVSIMTYDEFKHCWDTFVDHQGCPFQPW |
| | | DGLDEHSQALSGRLRAILQNQGNGTKDSTKDIPETPSKDDKKYSIGLAIGTNSVGWAVITDEYKVPSKKFKVLGNTDRHSIKKNLIGA |
| | | LLFDSGETAEATRLKRTARRRYTRRKNRICYLQEIFSNEMAKVDDSFFHRLEESFLVEEDKKHERHPIFGNIVDEVAYHEKYPTIYHL |
| | | RKKLVDSTDKADLRLIYLALAHMIKFRGHFLIEGDLNPDNSDVDKLFIQLVQTYNQLFEENPINASGVDAKAILSARLSKSRRLENLI |
| | | AQLPGEKKNGLFGNLIALSLGLTPNFKSNFDLAEDAKLQLSKDTYDDDLDNLLAQIGDQYADLFLAAKNLSDAILLSDILRVNTEITK |
| | | APLSASMIKRYDEHHQDLTLLKALVRQQLPEKYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSRFAWMTRKSEETITPWNFEEVVDKGASAQS |
| | | FIERMTNFDKNLPNEKVLPKHSLLYEYFTVYNELTKVKYVTEGMRKPAFLSGEQKKAIVDLLFKTNRKVTVKQLKEDYFKKIECFDSV |
| | | EISGVEDRFNASLGTYHDLLKIIKDKDFLDNEENEDILEDIVLTLTLFEDREMIEERLKTYAHLFDDKVMKQLKRRRYTGWGRLSRKL |
| | | INGIRDKQSGKTILDFLKSDGFANRNFMQLIHDDSLTFKEDIQKAQVSGQGDSLHEHIANLAGSPAIKKGILQTVKVVDELVKVMGRH |
| | | KPENIVIEMARENQTTQKGQKNSRERMKRIEEGIKELGSQILKEHPVENTQLQNEKLYLYYLQNGRDMYVDQELDINRLSDYDVDHIV |
| | | PQSFLKDDSIDNKVLTRSDKNRGKSDNVPSEEVVKKMKNYWRQLLNAKLITQRKFDNLTKAERGGLSELDKAGFIKRQLVETRQITKH |
| | | VAQILDSRMNTKYDENDKLIREVKVITLKSKLVSDFRKDFQFYKVREINNYHHAHDAYLNAVVGTALIKKYPKLESEFVYGDYKVYDV |
| | | RKMIAKSEQEIGKATAKYFFYSNIMNFFKTEITLANGEIRKRPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKGKSKKLKSVKELLGITIMERSSFEKNPIDFLEAKGYKEVKKDL |
| | | IIKLPKYSLFELENGRKRMLASAGELQKGNELALPSKYVNFLYLASHYEKLKGSPEDNEQKQLFVEQHKHYLDEIIEQISEFSKRVIL |
| | | ADANLDKVLSAYNKHRDKPIREQAENIIHLFTLTNLGAPAAFKYFDTTIDRKRYTSTKEVLDATLIHQSITGLYETRIDLSQLGGDGG |
| | | GSPKKKRKV |
| 859 | Exemplary AA sequence for UGI | MTNLSDIIEKETGKQLVIQESILMLPEEVEEVIGNKPESDILVHTAYDESTDENVMLLTSDAPEYKPWALVIQDSNGENKIKMLSGGS |
| | | KRTADGSEFESPKKKRKVE |
| 860 | Exemplary AA sequence for UGI with Hibit | MTNLSDIIEKETGKQLVIQESILMLPEEVEEVIGNKPESDILVHTAYDESTDENVMLLTSDAPEYKPWALVIQDSNGENKIKMLSGGS |
| | tab | KRTADGSEFESPKKKRKVESESATPESVSGWRLFKKIS |
| | 861-900 | Not used |
| 901 | exemplary XTEN | SGSETPGTSESATPES |
| 902 | exemplary XTEN | SGSETPGTSESA |
| 903 | exemplary XTEN | SGSETPGTSESATPEGGSGGS |
| 904 | Exemplary AA sequence for exemplary linker | GGGGSEAAAKEAAAK |
| 905 | Exemplary AA sequence for exemplary linker | EAAAKGGGGSGGGGS |
| 906 | Exemplary AA sequence for exemplary linker | EAAAKEAAAKEAAAK |
| 907 | Exemplary AA sequence for exemplary linker | GGGGSGGGGSGGGGSGGGGS |
| 908 | Exemplary AA sequence for exemplary linker | GGGGSGGGGSEAAAKEAAAK |
| 909 | Exemplary AA sequence for exemplary linker | GGGGSEAAAKGGGGSGGGGS |
| 910 | Exemplary AA sequence for exemplary linker | EAAAKEAAAKEAAAKGGGGSGGGGS |
| 911 | Exemplary AA sequence for exemplary linker | EAAAKEAAAKEAAAKEAAAK |
| 912 | Exemplary AA sequence for exemplary linker | GGGGSEAAAKEAAAKGGGGSEAAAK |
| 913 | Exemplary AA sequence for exemplary linker | EAAAKEAAAKGGGGSGGGGSGGGGS |
| 914 | Exemplary AA sequence for exemplary linker | EAAAKEAAAKGGGGSGGGGSEAAAK |
| 915 | Exemplary AA sequence for exemplary linker | SGGS |
| | SGGS | |
| 916 | Exemplary AA acid sequence for SV40 NLS | PKKKRKV |
| 917 | Exemplary NLS1 | LAAKRSRTT |
| 918 | Exemplary NLS2 | QAAKRSRTT |
| 919 | Exemplary NLS3 | PAPAKRERTT |
| 920 | Exemplary NLS4 | QAAKRPRTT |
| 921 | Exemplary NLS5 | RAAKRPRTT |
| 922 | Exemplary NLS6 | AAAKRSWSMAA |
| 923 | Exemplary NLS7 | AAAKRVWSMAF |
| 924 | Exemplary NLS8 | AAAKRSWSMAF |
| 925 | Exemplary NLS9 | AAAKRKYFAA |
| 926 | Exemplary NLS10 | RAAKRKAFAA |
| 927 | Exemplary NLS11 | RAAKRKYFAV |
| 928 | Alternate SV40 NLS | PKKKRRV |
| 929 | Nucleoplasmin NLS | KRPAATKKAGQAKKKK |
| 930 | Exemplary AA sequence for exemplary linker | GGS |
| 931 | Exemplary AA sequence for exemplary linker | GGGGS |
| 932 | Exemplary AA sequence for exemplary linker | EAAAK |
| 933 | Exemplary AA sequence for exemplary linker | SEGSA |
| 934 | Exemplary AA sequence for exemplary linker | SEGSAGTST |
| 935 | Exemplary AA sequence for exemplary linker | GGGGSGGGGS |
| 936 | Exemplary AA sequence for exemplary linker | GGGGSEAAAK |
| | sequence for | |
| | exemplary linker | |
| 937 | Exemplary AA sequence for exemplary linker | EAAAKGGGGS |
| 938 | Exemplary AA sequence for exemplary linker | EAAAKEAAAK |
| 939 | Exemplary AA sequence for exemplary linker | SEGSAGTSTESEGSA |
| 940 | Exemplary AA sequence for exemplary linker | GGGGSGGGGSGGGGS |
| 941 | Exemplary AA sequence for exemplary linker | GGGGSGGGGSEAAAK |
| 942 | Exemplary AA sequence for exemplary linker | GGGGSEAAAKGGGGS |
| 943 | Exemplary AA sequence for exemplary linker | EAAAKGGGGSEAAAK |
| 944 | Exemplary AA sequence for exemplary linker | EAAAKEAAAKGGGGS |
| 945 | Exemplary AA sequence for exemplary linker | SEGSAGTSTESEGSAGTSTE |
| 946 | Exemplary AA sequence for exemplary linker | GGGGSGGGGSGGGGSEAAAK |
| 947 | Exemplary AA sequence for exemplary linker | GGGGSGGGGSEAAAKGGGGS |
| 948 | Exemplary AA sequence for exemplary linker | GGGGSEAAAKGGGGSEAAAK |
| 949 | Exemplary AA sequence for exemplary linker | GGGGSEAAAKEAAAKGGGGS |
| 950 | Exemplary AA sequence for exemplary linker | GGGGSEAAAKEAAAKEAAAK |
| 951 | Exemplary AA sequence for exemplary linker | EAAAKGGGGSGGGGSGGGGS |
| 952 | Exemplary AA sequence for exemplary linker | EAAAKGGGGSGGGGSEAAAK |
| 953 | Exemplary AA sequence for exemplary linker | EAAAKGGGGSEAAAKGGGGS |
| 954 | Exemplary AA sequence for exemplary linker | EAAAKGGGGSEAAAKEAAAK |
| 955 | Exemplary AA sequence for exemplary linker | EAAAKEAAAKGGGGSGGGGS |
| 956 | Exemplary AA sequence for exemplary linker | EAAAKEAAAKGGGGSEAAAK |
| 957 | Exemplary AA sequence for exemplary linker | EAAAKEAAAKEAAAKGGGGS |
| 958 | Exemplary AA sequence for exemplary linker | SEGSAGTSTESEGSAGTSTESEGSA |
| 959 | Exemplary AA sequence for exemplary linker | GGGGGGGGSGGGGSGGGGSGGGGS |
| 960 | Exemplary AA sequence for exemplary linker | GGGGSGGGGSGGGGSGGGGSEAAAK |
| 961 | Exemplary AA sequence for exemplary linker | GGGGSGGGGSGGGGSEAAAKGGGGS |
| 962 | Exemplary AA sequence for exemplary linker | GGGGSGGGGSGGGGSEAAAKEAAAK |
| 963 | Exemplary AA sequence for exemplary linker | GGGGSGGGGSEAAAKGGGGSGGGGS |
| 964 | Exemplary AA sequence for exemplary linker | GGGGSGGGGSEAAAKGGGGSEAAAK |
| 965 | Exemplary AA sequence for exemplary linker | GGGGSGGGGSEAAAKEAAAKGGGGS |
| 966 | Exemplary AA sequence for exemplary linker | GGGGSGGGGSEAAAKEAAAKEAAAK |
| 967 | Exemplary AA sequence for exemplary linker | GGGGSEAAAKGGGGSGGGGSGGGGS |
| 968 | Exemplary AA sequence for exemplary linker | GGGGSEAAAKGGGGSGGGGSEAAAK |
| 969 | Exemplary AA sequence for exemplary linker | GGGGSEAAAKGGGGSEAAAKGGGGS |
| 970 | Exemplary AA sequence for exemplary linker | GGGGSEAAAKGGGGSEAAAKEAAAK |
| 971 | Exemplary AA sequence for exemplary linker | GGGGSEAAAKEAAAKGGGGSGGGGS |
| 972 | Exemplary AA sequence for exemplary linker | GGGGSEAAAKEAAAKEAAAKGGGGS |
| 973 | Exemplary AA sequence for exemplary linker | GGGGSEAAAKEAAAKEAAAKEAAAK |
| 974 | Exemplary AA sequence for exemplary linker | EAAAKGGGGSGGGGSGGGGSGGGGS |
| 975 | Exemplary AA sequence for exemplary linker | EAAAKGGGGSGGGGSGGGGSEAAAK |
| 976 | Exemplary AA sequence for exemplary linker | EAAAKGGGGSGGGGSEAAAKGGGGS |
| 977 | Exemplary AA sequence for exemplary linker | EAAAKGGGGSGGGGSEAAAKEAAAK |
| 978 | Exemplary AA sequence for exemplary linker | EAAAKGGGGSEAAAKGGGGGGGGS |
| 979 | Exemplary AA sequence for exemplary linker | EAAAKGGGGSEAAAKGGGGSEAAAK |
| 980 | Exemplary AA sequence for exemplary linker | EAAAKGGGGSEAAAKEAAAKGGGGS |
| 981 | Exemplary AA sequence for exemplary linker | EAAAKGGGGSEAAAKEAAAKEAAAK |
| 982 | Exemplary AA sequence for exemplary linker | EAAAKEAAAKGGGGSEAAAKGGGGS |
| 983 | Exemplary AA sequence for exemplary linker | EAAAKEAAAKGGGGSEAAAKEAAAK |
| 984 | Exemplary AA sequence for exemplary linker | EAAAKEAAAKEAAAKGGGGSEAAAK |
| 985 | Exemplary AA sequence for exemplary linker | EAAAKEAAAKEAAAKEAAAKGGGGS |
| 986 | Exemplary AA sequence for exemplary linker | EAAAKEAAAKEAAAKEAAAKEAAAK |
| 987 | Exemplary AA sequence for exemplary linker | GTKDSTKDIPETPSKD |
| 988 | Exemplary AA sequence for exemplary linker | GRDVRQPEVKEEKPES |
| 989 | Exemplary AA sequence for exemplary linker | EGKSSGSGSESKSTAG |
| 990 | Exemplary AA sequence for exemplary linker | TPGSPAGSPTSTEEGT |
| 991 | Exemplary AA sequence for exemplary linker | GSEPATSGSETPGTST |
| 1002 | G000529 (B2M) guide sequence | GGCCACGGAGCGAGACAUCU |
| 1003 | G000529 (B2M) full guide RNA sequence | GGCCACGGAGCGAGACAUCUGUUUUAGAGCUAGAAAUAGCAAGUUAAAAUAAGGCUAGUCCGUUAUCAACUUGAAAAAGUGGCACCGA |
| | | GUCGGUGCUUUU |
| 1004 | G000529 (B2M) modified guide RNA sequence | mG*mG*mC*CACGGAGCGAGACAUCUGUUUUAGAmGmCmUmAmGmAmAmAmUmAmGmCAAGUUAAAAUAAGGCUAGUCCGUUAUCAmA |
| | | mCmUmUmGmAmAmAmAmAmGmUmGmGmCmAmCmCmGmAmGmUmCmGmGmUmGmCmU*mU*mU*mU |

*The guide sequence described in this Table may be unmodified, modified with the exemplary modification pattern shown in the Table, or modified with a different
modification pattern disclosed herein or available in the art.

### EXAMPLES

The following examples are provided to illustrate certain disclosed embodiments and are not to be construed as limiting the scope of this disclosure in any way.

### Example 1: General Methods

### 1.1 Preparation of Lipid Nanoparticles

The lipid components were dissolved in 100% ethanol at various molar ratios. The RNA cargos (e.g., Cas9 mRNA and sgRNA) were dissolved in 25 mM citrate buffer, 100 mM NaCl, pH 5.0, resulting in a concentration of RNA cargo of approximately 0.45 mg/mL.

The lipid nucleic acid assemblies contained ionizable Lipid A ((9Z,12Z)-3-((4,4-bis(octyloxy) butanoyl)oxy)-2-((((3-(diethylamino) propoxy) carbonyl)oxy) methyl) propyl octadeca-9,12-dienoate, also called 3-((4,4-bis(octyloxy) butanoyl)oxy)-2-((((3-(diethylamino) propoxy) carbonyl)oxy) methyl) propyl (9Z,12Z)-octadeca-9,12-dienoate), lipid nanoparticles used 35 Lipid A: 47.5 cholesterol: 15 DSPC: 2.5 PEG2k-DMG by molarity. The lipid nucleic acid assemblies were formulated with a lipid amine to RNA phosphate (N: P) molar ratio of about 6, and a ratio of gRNA to mRNA of 1:1 by weight.

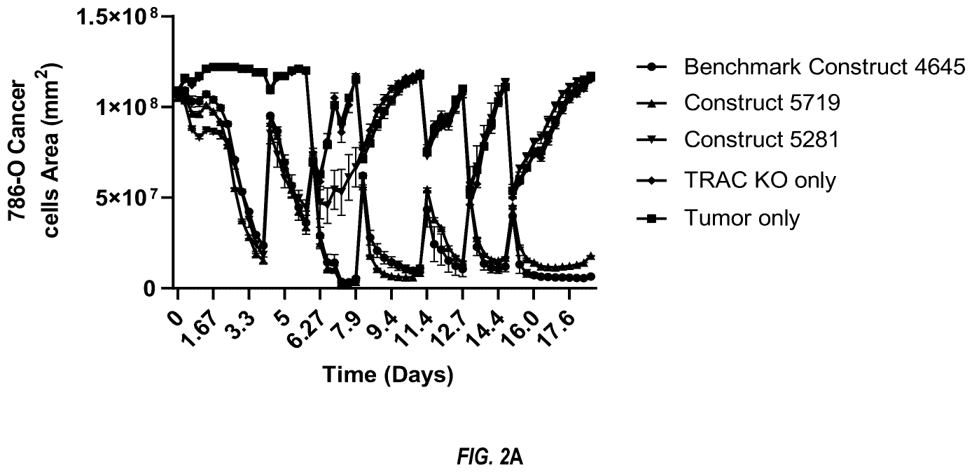

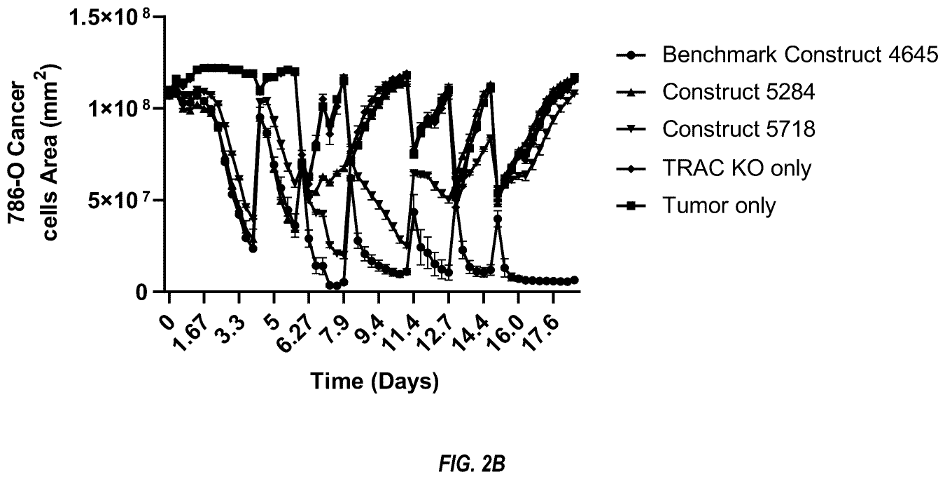

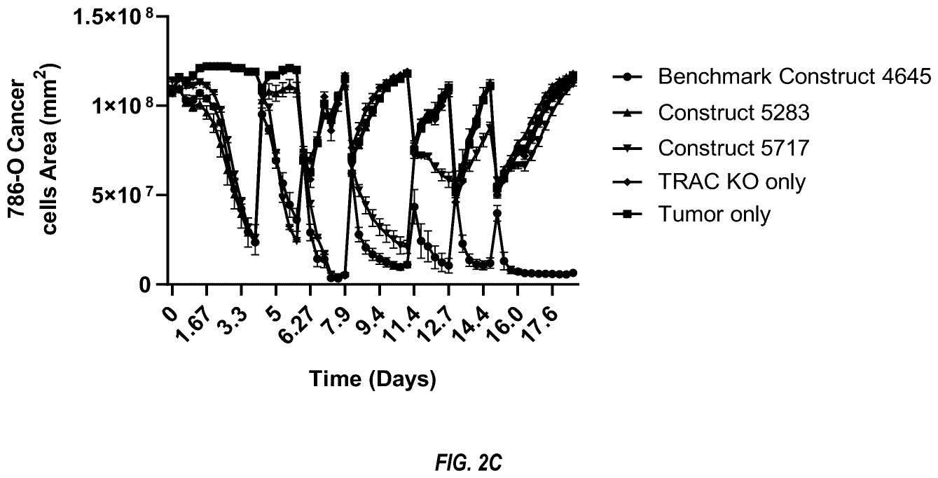

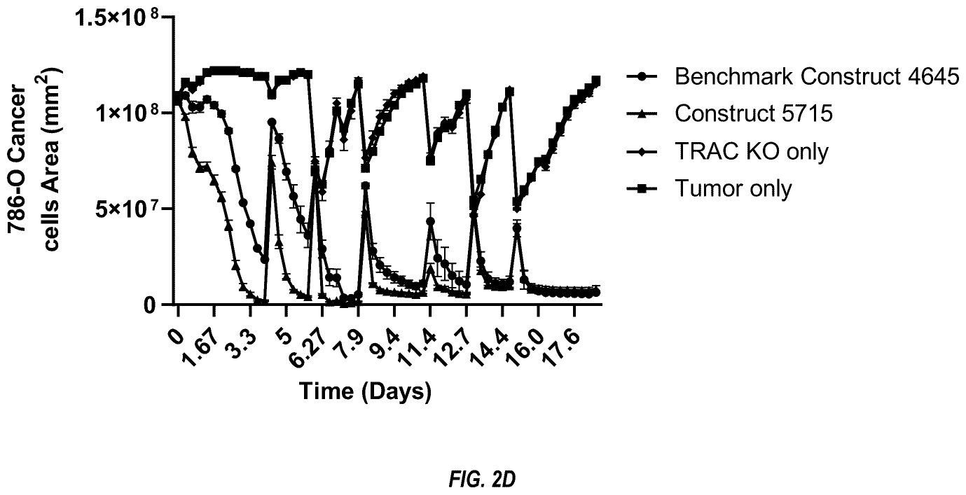

Lipid nanoparticles (LNP compositions) were prepared using a cross-flow technique utilizing impinging jet mixing of the lipid in ethanol with two volumes of RNA solutions and one volume of water. The lipids in ethanol were mixed through a mixing cross with the two volumes of RNA solution. A fourth stream of water was mixed with the outlet stream of the cross through an inline tee (See WO2016010840 FIG. 2.). The LNP compositions were held for 1 hour at room temperature (RT), and further diluted with water (approximately 1:1 v/v). LNP compositions were concentrated using tangential flow filtration on a flat sheet cartridge (Sartorius, 100 kD MWCO) and buffer exchanged using PD-10 desalting columns (GE) into 50 mM Tris, 45 mM NaCl, 5% (w/v) sucrose, pH 7.5 (TSS). Alternatively, the LNP's were optionally concentrated using 100 kDa Amicon spin filter and buffer exchanged using PD-10 desalting columns (GE) into TSS. The resulting mixture was then filtered using a 0.2 μm sterile filter. The final LNP was stored at 4° C. or −80° C. until further use.

1.2 In Vitro Transcription (IVT) of mRNA

Capped and polyadenylated mRNA containing N1-methyl pseudo-U was generated by in vitro transcription (IVT) using a linearized plasmid DNA template and T7 RNA polymerase. Plasmid DNA containing a T7 promoter, a sequence for transcription, and a polyadenylation sequence was linearized by incubating at 37° C. for 2 hours with Xbal with the following conditions: 200 ng/μL plasmid, 2 U/μL Xbal (NEB), and 1× reaction buffer. The Xbal was inactivated by heating the reaction at 65° C. for 20 min. The linearized plasmid was purified from enzyme and buffer salts. The IVT reaction to generate modified mRNA was performed by incubating 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer at 37° C. for 1.5-4 hours. TURBO DNase (Thermo Fisher) was added to a final concentration of 0.01 U/μL, and the reaction was incubated for an additional 30 minutes to remove the DNA template. The mRNA was purified using a MegaClear Transcription Clean-up kit (Thermo Fisher) or a RNeasy Maxi kit (Qiagen) per the manufacturers' protocols.

Alternatively, the mRNA was purified through a precipitation protocol, which in some cases was followed by HPLC-based purification. Briefly, after the DNase digestion, mRNA was purified using LiCl precipitation, ammonium acetate precipitation and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In an alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

The mRNAs encoding a SpyCas9, an NmeCas9, or a base editor described herein were generated from plasmid DNA encoding an open reading frame having the sequence of one of SEQ ID NOs: 801-828 (see sequences in Table 10). When SEQ ID NOs: 801-828 are referred to below with respect to RNAs, it is understood that Ts should be replaced with Us (e.g., N1-methyl pseudouridine as described above). Messenger RNAs used in the Examples include a 5′ cap and a 3′ polyadenylation region, e.g., up to 100 nucleotides, and have a nucleic acid sequence of one of SEQ ID NOs: 801-828 in Table 10. Guide RNAs were chemically synthesized by methods known in the art.

### 1.3 Next-Generation Sequencing (“NGS”) and Analysis for Editing Efficiency

DNA was extracted using a commercial kit according to the manufacturer's protocol, for example QuickExtract™ DNA Extraction Solution (Lucigen, Cat. QE09050). To quantitatively determine the efficiency of editing at the target location in the genome, deep sequencing was utilized to identify the presence of insertions and deletions introduced by gene editing. PCR primers were designed around the target site within the gene of interest (e.g., TRAC) and the genomic area of interest was amplified. Primer sequence design was done as is standard in the field. Additional PCR was performed according to the manufacturer's protocols (e.g., Illumina, PacBio) to add chemistry for sequencing. The amplicons were sequenced on a NGS platform (e.g., Illumina, PacBio). The reads were aligned to the reference genome (e.g., hg38) after eliminating those having low quality scores (PHRED score <20). Reads that overlapped the target region of interest were re-aligned to the local genome sequence to improve the alignment.

### 1. 1.3.1 Indel Analysis

The number of wild-type reads versus the number of reads which contained indels was calculated. Insertions and deletions were scored in a 20 bp region centered on the predicted Cas9 cleavage site. Indel percentage is defined as the total number of sequencing reads with one or more bases inserted or deleted within the 20 bp scoring region divided by the total number of sequencing reads, including wild type.

### 2. 1.3.2 Base Editing Analysis

The number of wild type reads versus the number of reads which contain C-to-T mutations, or C-to-A/G mutations was calculated. C-to-T mutations or C-to-A/G mutations were scored in a 40 bp region including 10 bp upstream and 10 bp downstream of the 20 bp sgRNA target sequence. The C-to-T editing percentage is defined as the total number of sequencing reads with either one or more C-to-T mutations within the 40 bp region divided by the total number of sequencing reads, including wild type. The percentage of C-to-A/G mutations are calculated similarly.

### Example 2-Re-Challenging Anti-CD70 CAR-T Cells with 786-O Tumor Cells

### 2.1 Thawing and Resting Anti-CD70 CAR-T and Control T-Cells

Anti-CD70 CAR-T cells engineered using different CD70 antigen-binding proteins and costimulatory domains were utilized in the serial tumor re-challenge assay to assess the efficacy of each anti-CD70 CAR construct. T-cells subjected to the re-challenge assay were engineered to express the following anti-CD70 CAR constructs: Benchmark Construct 4645, Construct 5715, Construct 5717, Construct 5718, Construct 5719 (SEQ ID NO: 106), Construct 5281, Construct 5283, and Construct 5284. A tumor-only sample and a T-cell engineered with TRAC KO only were compared as controls.

On Day 0, the anti-CD70 CAR-T cell cryopreserved vials were thawed in the 37° C. water bath and transferred to a 15 mL conical tube containing 9 mL of pre-warmed T-cell growth media (TCGM). The T-cell growth media consists of: CTS OpTmizer T-cell expansion SFM (Gibco, Cat. #A3705001), OpTmizer CTS T-cell expansion supplement (Gibco, Cat. #A2596101), 5% GemCell human Ab serum (GeminiBio, Cat. #100-512), 1% HEPES (Gibco, Cat. #15630-080), 1% GlutaMAX supplement (Gibco, Cat. #35050-061), 1% Penicillin-Streptomycin (10,000 U/mL; Gibco, Cat. #15140122) and further supplemented with 100 U/mL of recombinant human interleukin-2 (Peprotech, Cat. 200-02), 5 ng/ml IL-7 (Peprotech, Cat. 200-07), and 5 ng/ml IL-15 cytokines (Peprotech, Cat. 200-15). 1 mL of TCGM media was added to the empty vials and transferred to the 15 mL conical tubes to obtain residual cell suspension. The 15 mL conical tubes were centrifuged at 300 RCF for 3 minutes at room temperature. After centrifugation, the supernatant was aspirated, and the cell pellet was resuspended in TCGM media with cytokines. An aliquot was obtained for cell counting using the Cellaca instrument. The anti-CD70 CAR T cells were adjusted at a concentration of 1.0×10⁶ cells/mL. The T-cells were transferred to T-75 flasks and incubated overnight in a 37° C. incubator.

2.2 Assay Setup with Effector (Anti-CD70 CAR-T) and Target (Tumor) Cells

On Day 0, 786-O GFP-Luciferase tumor cells were harvested from T-75 flasks and counted using the Cellaca counting instrument. (786-O tumor cell media composed of-RPMI 1640 Medium GlutaMAX™ Supplement, (Gibco, Cat. #61870127), 10% Fetal Bovine Serum (FBS; Gibco, Cat #A3160501), 1% Penicillin-Streptomycin (Gibco, Cat. #15070-063). After cell counting, 250,000 786-O tumor cells were plated per well in a sterile 24-well clear TC-treated flat-bottom plate (Corning, Cat. 353047). After plating the 24-well plates were incubated overnight in a 37° C. incubator to allow tumor cells to adhere to the plate.

After 24 hours rest, all CAR-T cells were harvested from T-75 flasks into 50 mL conical tubes. The T-cells were centrifuged at 300 RCF for 3 minutes at room temperature and resuspended in 5 mL of TCGM media without cytokines. The T-cells were aliquoted for cell counting using the Cellaca instrument. After cell counting, the CAR-T cells were normalized to the lowest anti-CD70 CAR expression. After normalization, CAR T-cells were added to the 786-O tumor cells in 24-well flat-bottom plates maintaining the starting effector to target ratio of 1:3 for 786-O. After plating the plate was transferred to a live cell analysis instrument (Incuyte S3, Sartorius) at 37° C.; the images were captured every four hours via Incucyte software using Phase and Green channels with 10× objective.

2.3 Re-Challenge with 786-O Tumor Cells

After every two to three days, the anti-CD70 CAR-T cells were re-challenged with tumor cells. Six hours prior to re-challenge, 150,000 786-O tumor cells were freshly plated per well in a sterile 24-well flat-bottom plate and were incubated in the 37° C. incubator to allow tumor cells to adhere to the plate. After six hours, the 24-well plates were removed from the Incucyte instrument and centrifuged at 300 RCF for 5 minutes. After centrifugation, 1 mL of the supernatant was removed from the 24 well plates and the remaining 1 mL of media containing expanded anti-CD70 CAR-T cells was transferred to the freshly plated 786-O tumor cells. After plating the plate was transferred to a live cell analysis instrument (Incuyte S3, Sartorius) at 37° C.; the images were captured every four hours via Incucyte software using Phase and Green channels with 10× objective. FIGS. 2A-D show the 786-0 tumor cells area (mm2) for the different engineered anti-CD70 CAR-T cells.

### Example 3—In Vivo Efficacy Study of Engineered Anti-CD70 CAR-T Cells

Using the antigenhigh 786-O Renal Cell Carcinoma (RCC) tumor model, female NOG mice were engrafted with 10x106 786-O tumor cells. When the solid tumors reached an average volume of 400-450 mm3, anti-CD70 CAR-T cells engineered with constructs 4645 (Benchmark), 5715, 5719 (SEQ ID NO: 106), 5718, 5717 or 5281 were injected into the mice. The ability of each engineered anti-CD70 CAR-T cells to control solid tumors was assessed.

### 3.1 Preparation of Anti-CD70 CAR-T Cells for the In Vivo Study

T-cells were isolated from peripheral blood of healthy human donor W3410 with the following MHC I phenotype: HLA-A*02:01:01G, 03:01:01G, HLA-B*07:02:01G, 40:01:02, HLA-C*03:04:01, 07:02:01G, HLA-DRB1*15:01:01, 15:01:01, HLA-DRB5*01:01:01, HLA-DQA1*01:02:01, 02:01:01, HLA-DQB1*06:02:01, 06:02:01, HLA-DPA1-01:03:01, HLA-DPB1*04:01:01. Briefly, a leukapheresis pack (HemaCare Technologies) was treated in ammonium chloride RBC lysis buffer (Stemcell Technologies, Cat. 07800) to lyse red blood cells. Peripheral blood mononuclear cell (PBMC) count was determined post lysis and T-cell isolation was performed using EasySep Human T cell isolation kit (Stemcell Technologies, Cat. 17951) according to manufacturer's protocol. Isolated CD3+ T-cells were re-suspended in Cryostor CS10 media (Stemcell Technologies, Cat. 07930) and frozen down in liquid nitrogen until further use. Frozen T-cells were thawed at a cell concentration of 1×10⁶ cells/mL into T-cell growth media (TCGM). Cells were rested at 37° C. for 24 hours.

24 post thawing T-cells were counted and resuspended at 1×10⁶ cells/mL in T-cell activation media (TCAM) composed of CTS Optimizer (Gibco A3705001), GlutaMAX (Gibco Cat. 35050061), 2.5% Human AB Serum (Gemini Cat. 100-512), HEPES (Gibco Cat. 15630080), Penicillin-Streptomycin (Cat. 15140122), 200 U/mL of recombinant human interleukin-2 (Peprotech, Cat. 200-02), 5 ng/ml IL-7 (Peprotech, Cat. 200-07), and 5 ng/ml IL-15 (Peprotech, Cat. 200-15) as well as TransACT (Miltenyi Cat. 130-111-160) added to a final concentration of 1/100 of the total volume. 48 hours post activation all groups were transduced with EF1α-CAR adeno-associated virus (AAV). AAV was removed from −80° C. and thawed on ice. Transduction media was generated from TCAM by adding ApoE3 (Peprotech, Cat. 350-02) to a final concentration of 2.5 μg/mL, TRAC-targeted LNP (Guide ID: G013006) to a final concentration of 2.5 μg/mL, and DNApki Compound 1 to a final concentration of 0.25 μM. Cells were collected, centrifuged at 500×g for 5 minutes and resuspended at 0.5e6cells/mL in transduction media. Appropriate AAVs for anti-CD70 CAR constructs were added to cells at an MOI of 3e5 GC/cell. Cells were mixed and were incubated at 37° C. for 24 hours.

24 hours post TRAC-LNP and AAV treatment, cells were resuspended to a concentration of 0.5e6 cells/mL in fresh T-cell media with cytokines, recombinant human interleukin-2 (Peprotech, Cat. 200-02), IL-7 (Peprotech, Cat. 200-07), and IL-15 (Peprotech, Cat. 200-15) and cells were incubated at 37° C. for 24 hours.

24 hours later, cells were transferred to GREX plates (Wilson Wolf Cat. 80660M) and expanded for ten days with regular changes in media and cytokines. After expansion, CAR insertion rates were quantified using flow cytometry, and cells were cryopreserved in Cryostor CS10 freezing media (StemCell Cat. 07930).

### 3.2 Anti-CD70 CAR Constructs 5715 and 5719 Induced Complete Tumor Control in 786-O Model Comparable to the Benchmark Anti-CD70 CAR

For the in vivo study, 786-O-GFP cells were harvested from culture, washed twice with Hanks' Balanced Salt Solution (HBSS; Gibco, Cat. No. 14025-092) and resuspended at 10×10⁶ cells in 400 μl of HBSS. 50 female NOG mice (Taconic) were dosed subcutaneously on the right flank of the animals. The animals were monitored two times a week for tumor growth by caliper measurements and their tumor volumes were recorded. Once the tumors reached an average of 400-450 mm³ in volume, tumor bearing animals were randomized followed by T-cell injection on day 23 post tumor engraftment. Engineered anti-CD70 CAR T cells were thawed, washed with HBSS (Gibco, Cat. No. 14025-092) and resuspended at 3×10⁶ cells and 1×10⁶ cells in 150 μl of HBSS for injections. Five mice per T-cell group were dosed by tail vein injections in the tumor engrafted animals.

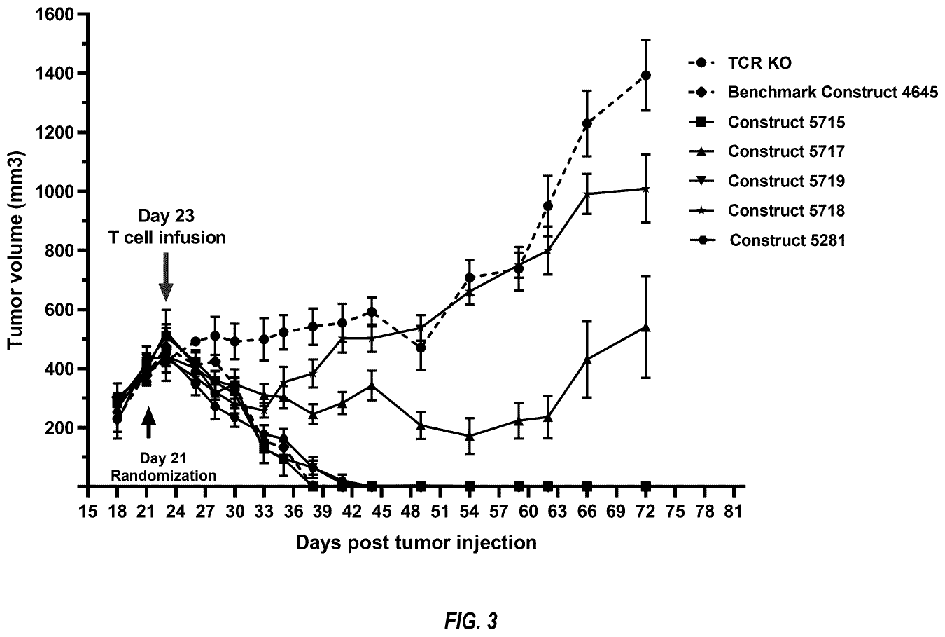

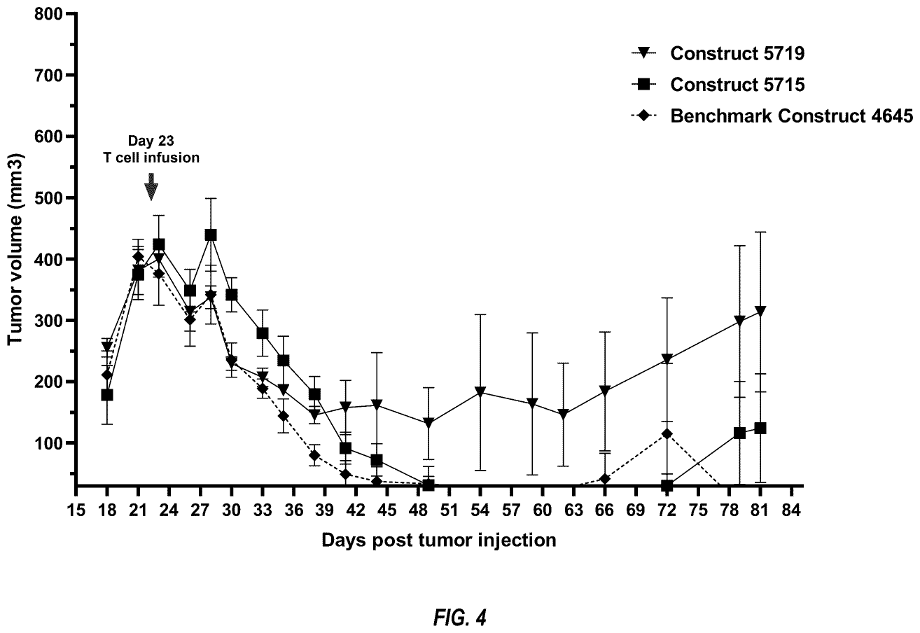

Caliper measurements of the tumors and body weight measurements were taken two or three times a week post T cell infusion on days 0, 3, 5, 7, 10, 12, 15, 18, 21, 26, 31, 36, 39, 43, 49, 56, and 58 for the 1×106 dosed group. For the 3×10⁶ group, measurements were taken at the same time intervals, but only reached day 49 post T-cell infusion. The mice were prepared to be measured by restraining the animal securely and shaving the excess fur off the right-side flank of the animal. The shaved area was then wiped down with an alcohol swab to clearly visualize the tumor and the length and the width of the tumor was measured using calipers. Tumor volumes were calculated as (((Length+Width)/2/2)\hat{ }3)*3.14*1.33. Tables 11 and 12 and FIGS. 3 and 4 show the mean tumor volume data for each group dosed with different constructs from day of randomization (Day 21 post tumor engraftment) until study termination. All of the 3×10⁶ dosed groups were euthanized on day 72 post tumor engraftment. The 1×10⁶ dosed groups were euthanized on day 82 post tumor engraftment.

At 3×10⁶ dose, animals that received T-cells with construct 5715 and benchmark construct 4645 reached complete tumor regression 15 days post T-cell dosing and remained tumor-free until the end of the study. T-cells with construct 5719 and construct 5281 induced complete tumor clearance 18 days post T-cell infusion and the animals remained tumor-free until the end of the study. T-cells with construct 5717 and construct 5718 exhibited partial tumor control.

At 1×10⁶ dose, the benchmark construct 4645 induced complete tumor clearance 21 days post T-cell dosing. Construct 5715 performed comparably by inducing total regression 26 days post T-cell infusion. Two out of five animals in the group treated with construct 5719 reached tumor clearance on day 21 and day 26, respectively.

TABLE 11
Average tumor volumes from all groups post 3e6 T-cell dosing.

| | | | | | |
|---|---|---|---|---|---|
| | Days post | Days post T | Average | SD of | |
| T cell dose | engraftment | cell dosing | TVols | TVols | N |

TCR KO

| | | | | | |
|---|---|---|---|---|---|
| 3e6/animal | 21 | −2 | 386.204 | 73.8 | 5 |
| 3e6/animal | 23 | 0 | 424.944 | 79.7 | 5 |
| 3e6/animal | 26 | 3 | 491.79 | 30.2 | 5 |
| 3e6/animal | 28 | 5 | 510.184 | 129.3 | 5 |
| 3e6/animal | 30 | 7 | 491.804 | 121.7 | 5 |
| 3e6/animal | 33 | 10 | 498.958 | 143.3 | 5 |
| 3e6/animal | 35 | 12 | 522.548 | 116.4 | 5 |
| 3e6/animal | 38 | 15 | 541.416 | 123.4 | 5 |
| 3e6/animal | 41 | 18 | 554.474 | 130.1 | 5 |
| 3e6/animal | 44 | 21 | 591.874 | 98.8 | 5 |
| 3e6/animal | 49 | 26 | 469.864 | 148.2 | 5 |
| 3e6/animal | 54 | 31 | 707.794 | 119.0 | 5 |
| 3e6/animal | 59 | 36 | 737.794 | 148.0 | 5 |
| 3e6/animal | 62 | 39 | 949.884 | 205.5 | 5 |
| 3e6/animal | 66 | 43 | 12229.69 | 221.9 | 5 |
| 3e6/animal | 72 | 49 | 1392.922 | 239.2 | 5 |

Benchmark Construct 4645

| | | | | | |
|---|---|---|---|---|---|
| 3e6/animal | 21 | −2 | 376.54 | 59.1 | 4 |
| 3e6/animal | 23 | 0 | 472.89 | 87.3 | 4 |
| 3e6/animal | 26 | 3 | 410.68 | 25.9 | 4 |
| 3e6/animal | 28 | 5 | 423.54 | 27.6 | 4 |
| 3e6/animal | 30 | 7 | 342.41 | 45.8 | 4 |
| 3e6/animal | 33 | 10 | 152.90 | 18.0 | 4 |
| 3e6/animal | 35 | 12 | 133.4 | 18.4 | 4 |
| 3e6/animal | 38 | 15 | 2.177 | 3.8 | 4 |
| 3e6/animal | 41 | 18 | 0 | 0 | 4 |
| 3e6/animal | 44 | 21 | 0 | 0 | 4 |
| 3e6/animal | 49 | 26 | 0 | 0 | 4 |
| 3e6/animal | 54 | 31 | 0 | 0 | 4 |
| 3e6/animal | 59 | 36 | 0 | 0 | 4 |
| 3e6/animal | 62 | 39 | 0 | 0 | 4 |
| 3e6/animal | 66 | 43 | 0 | 0 | 4 |
| 3e6/animal | 72 | 49 | 0 | 0 | 4 |

Construct 5715

| | | | | | |
|---|---|---|---|---|---|
| 3e6/animal | 21 | −2 | 413.37 | 73.8 | 5 |
| 3e6/animal | 23 | 0 | 507.614 | 83.6 | 5 |
| 3e6/animal | 26 | 3 | 422.326 | 79.9 | 5 |
| 3e6/animal | 28 | 5 | 358.32 | 68.1 | 5 |
| 3e6/animal | 30 | 7 | 340.42 | 54.4 | 5 |
| 3e6/animal | 33 | 10 | 127.902 | 30.7 | 5 |
| 3e6/animal | 35 | 12 | 93.576 | 25.9 | 5 |
| 3e6/animal | 38 | 15 | 0 | 0 | 5 |
| 3e6/animal | 41 | 18 | 0 | 0 | 5 |
| 3e6/animal | 44 | 21 | 0 | 0 | 5 |
| 3e6/animal | 49 | 26 | 0 | 0 | 5 |
| 3e6/animal | 54 | 31 | 0 | 0 | 5 |
| 3e6/animal | 59 | 36 | 0 | 0 | 5 |
| 3e6/animal | 62 | 39 | 0 | 0 | 5 |
| 3e6/animal | 66 | 43 | 0 | 0 | 5 |
| 3e6/animal | 72 | 49 | 0 | | 5 |

Construct 5717

| | | | | | |
|---|---|---|---|---|---|
| 3e6/animal | 21 | −2 | 396.628 | 76.5 | 5 |
| 3e6/animal | 23 | 0 | 525.138 | 146.3 | 5 |
| 3e6/animal | 26 | 3 | 408.148 | 95.2 | 5 |
| 3e6/animal | 28 | 5 | 317.392 | 96.3 | 5 |
| 3e6/animal | 30 | 7 | 347.206 | 100.1 | 5 |
| 3e6/animal | 33 | 10 | 310.46 | 75.2 | 5 |
| 3e6/animal | 35 | 12 | 302.748 | 75.2 | 5 |
| 3e6/animal | 38 | 15 | 245.238 | 67.4 | 5 |
| 3e6/animal | 41 | 18 | 283.366 | 74.5 | 5 |
| 3e6/animal | 44 | 21 | 342.772 | 99.8 | 5 |
| 3e6/animal | 49 | 26 | 207.276 | 91.0 | 5 |
| 3e6/animal | 54 | 31 | 171.512 | 120.4 | 5 |
| 3e6/animal | 59 | 36 | 223.72 | 122.2 | 5 |
| 3e6/animal | 62 | 39 | 235.582 | 144.5 | 5 |
| 3e6/animal | 66 | 43 | 430.278 | 258.0 | 5 |
| 3e6/animal | 72 | 49 | 540.764 | 344.7 | 5 |

Construct 5718

| | | | | | |
|---|---|---|---|---|---|
| 3e6/animal | 21 | −2 | 389.8 | 72.7 | 5 |
| 3e6/animal | 23 | 0 | 434.04 | 51.2 | 5 |
| 3e6/animal | 26 | 3 | 369.374 | 35.2 | 5 |
| 3e6/animal | 28 | 5 | 321.302 | 53.2 | 5 |
| 3e6/animal | 30 | 7 | 279.828 | 63.4 | 5 |
| 3e6/animal | 33 | 10 | 257.872 | 49.8 | 5 |
| 3e6/animal | 35 | 12 | 353.718 | 104.7 | 5 |
| 3e6/animal | 38 | 15 | 383.016 | 95.2 | 5 |
| 3e6/animal | 41 | 18 | 502.35 | 95.6 | 5 |
| 3e6/animal | 44 | 21 | 502.41 | 92.2 | 5 |
| 3e6/animal | 49 | 26 | 537.734 | 86.7 | 5 |
| 3e6/animal | 54 | 31 | 660.356 | 89.9 | 5 |
| 3e6/animal | 59 | 36 | 750.176 | 85.8 | 5 |
| 3e6/animal | 62 | 39 | 799.51 | 162.5 | 5 |
| 3e6/animal | 66 | 43 | 991.082 | 135.3 | 5 |
| 3e6/animal | 72 | 49 | 1008.9 | 230.9 | 5 |

Construct 5719

| | | | | | |
|---|---|---|---|---|---|
| 3e6/animal | 21 | −2 | 430.082 | 87.8 | 5 |
| 3e6/animal | 23 | 0 | 441.628 | 66.1 | 5 |
| 3e6/animal | 26 | 3 | 400.612 | 109.1 | 5 |
| 3e6/animal | 28 | 5 | 354.236 | 73.5 | 5 |
| 3e6/animal | 30 | 7 | 315.394 | 82.4 | 5 |
| 3e6/animal | 33 | 10 | 129.648 | 98.7 | 5 |
| 3e6/animal | 35 | 12 | 95.042 | 115.7 | 5 |
| 3e6/animal | 38 | 15 | 65.022 | 73.9 | 5 |
| 3e6/animal | 41 | 18 | 10.538 | 13.7 | 5 |
| 3e6/animal | 44 | 21 | 2.962 | 5.9 | 5 |
| 3e6/animal | 49 | 26 | 3.47 | 6.9 | 5 |
| 3e6/animal | 54 | 31 | 1.47 | 2.9 | 5 |
| 3e6/animal | 59 | 36 | 0 | 0.0 | 5 |
| 3e6/animal | 62 | 39 | 0 | 0.0 | 5 |
| 3e6/animal | 66 | 43 | 0 | 0.0 | 5 |
| 3e6/animal | 72 | 49 | 0 | 0.0 | 5 |

Construct 5281

| | | | | | |
|---|---|---|---|---|---|
| 3e6/animal | 21 | −2 | 375.7133 | 30.9 | 4 |
| 3e6/animal | 23 | 0 | 447.8325 | 155.4 | 4 |
| 3e6/animal | 26 | 3 | 345.1625 | 62.8 | 4 |
| 3e6/animal | 28 | 5 | 271.2925 | 76.3 | 4 |
| 3e6/animal | 30 | 7 | 234.0575 | 54.2 | 4 |
| 3e6/animal | 33 | 10 | 177.325 | 53.5 | 4 |
| 3e6/animal | 35 | 12 | 162.31 | 58.1 | 4 |
| 3e6/animal | 38 | 15 | 64.3725 | 42.0 | 4 |
| 3e6/animal | 41 | 18 | 20.55 | 35.6 | 4 |
| 3e6/animal | 44 | 21 | 0 | 0.0 | 4 |
| 3e6/animal | 49 | 26 | 0 | 0.0 | 4 |
| 3e6/animal | 54 | 31 | 0 | 0.0 | 4 |
| 3e6/animal | 59 | 36 | 0 | 0.0 | 4 |
| 3e6/animal | 62 | 39 | 0 | 0.0 | 4 |
| 3e6/animal | 66 | 43 | 0 | 0.0 | 4 |
| 3e6/animal | 72 | 49 | 0 | 0.0 | 4 |

TABLE 12
Average tumor volumes from all groups post 1e6 T cell dosing.

| | | | | | |
|---|---|---|---|---|---|
| | Days post | Days post T | Average | SD of | |
| T cell dose | engraftment | cell dosing | TVols | TVols | N |

Benchmark Construct 4645

| | | | | | |
|---|---|---|---|---|---|
| 1e6/animal | 21 | −2 | 403.88 | 49.2 | 5 |
| 1e6/animal | 23 | 0 | 376.06 | 102.7 | 5 |
| 1e6/animal | 26 | 3 | 301.084 | 86.4 | 5 |
| 1e6/animal | 28 | 5 | 341.934 | 95.8 | 5 |
| 1e6/animal | 30 | 7 | 235.304 | 55.8 | 5 |
| 1e6/animal | 33 | 10 | 189.012 | 31.4 | 5 |
| 1e6/animal | 35 | 12 | 144.362 | 55.3 | 5 |
| 1e6/animal | 38 | 15 | 80.066 | 34.3 | 5 |
| 1e6/animal | 41 | 18 | 49.254 | 44.2 | 5 |
| 1e6/animal | 44 | 21 | 37.38 | 47.9 | 5 |
| 1e6/animal | 49 | 26 | 33.48 | 56.4 | 5 |
| 1e6/animal | 54 | 31 | 25.674 | 51.3 | 5 |
| 1e6/animal | 59 | 36 | 23.698 | 47.4 | 5 |
| 1e6/animal | 62 | 39 | 25.49 | 51.0 | 5 |
| 1e6/animal | 66 | 43 | 41.626 | 83.3 | 5 |
| 1e6/animal | 72 | 49 | 115.04 | 230.1 | 5 |
| 1e6/animal | 79 | 56 | 0 | 0.0 | 4 |
| 1e6/animal | 81 | 58 | 0 | 0.0 | 4 |

Construct 5715

| | | | | | |
|---|---|---|---|---|---|
| 1e6/animal | 21 | −2 | 374.518 | 81.8 | 5 |
| 1e6/animal | 23 | 0 | 424.052 | 94.2 | 5 |
| 1e6/animal | 26 | 3 | 348.538 | 69.8 | 5 |
| 1e6/animal | 28 | 5 | 439.606 | 118.6 | 5 |
| 1e6/animal | 30 | 7 | 341.648 | 56.0 | 5 |
| 1e6/animal | 33 | 10 | 279.054 | 75.8 | 5 |
| 1e6/animal | 35 | 12 | 234.682 | 78.8 | 5 |
| 1e6/animal | 38 | 15 | 179.92 | 57.8 | 5 |
| 1e6/animal | 41 | 18 | 91.784 | 52.2 | 5 |
| 1e6/animal | 44 | 21 | 72.41 | 52.8 | 5 |
| 1e6/animal | 49 | 26 | 31.124 | 29.0 | 5 |
| 1e6/animal | 54 | 31 | 24.306 | 30.2 | 5 |
| 1e6/animal | 59 | 36 | 13.316 | 20.3 | 5 |
| 1e6/animal | 62 | 39 | 2.416 | 4.8 | 5 |
| 1e6/animal | 66 | 43 | 3.444 | 6.9 | 5 |
| 1e6/animal | 72 | 49 | 30.538 | 38.0 | 5 |
| 1e6/animal | 79 | 56 | 116.39 | 168.5 | 5 |
| 1e6/animal | 81 | 58 | 124.212 | 176.7 | 5 |

Construct 5719

| | | | | | |
|---|---|---|---|---|---|
| 1e6/animal | 21 | −2 | 381.426 | 78.3 | 5 |
| 1e6/animal | 23 | 0 | 399.928 | 59.0 | 5 |
| 1e6/animal | 26 | 3 | 314.068 | 63.7 | 5 |
| 1e6/animal | 28 | 5 | 337.57 | 36.9 | 5 |
| 1e6/animal | 30 | 7 | 229.668 | 21.8 | 5 |
| 1e6/animal | 33 | 10 | 206.976 | 30.0 | 5 |
| 1e6/animal | 35 | 12 | 185.784 | 17.7 | 5 |
| 1e6/animal | 38 | 15 | 145.43 | 28.6 | 5 |
| 1e6/animal | 41 | 18 | 157.758 | 88.8 | 5 |
| 1e6/animal | 44 | 21 | 161.622 | 171.9 | 5 |
| 1e6/animal | 49 | 26 | 131.688 | 117.6 | 5 |
| 1e6/animal | 54 | 31 | 182.218 | 254.1 | 5 |
| 1e6/animal | 59 | 36 | 163.676 | 231.6 | 5 |
| 1e6/animal | 62 | 39 | 146.058 | 168.5 | 5 |
| 1e6/animal | 66 | 43 | 184.27 | 193.8 | 5 |
| 1e6/animal | 72 | 49 | 235.97 | 201.3 | 5 |
| 1e6/animal | 79 | 56 | 298.058 | 247.3 | 5 |
| 1e6/animal | 81 | 58 | 313.63 | 260.2 | 5 |

### Example 4-In Vitro Assessment of Anti-CD70 CAR Constructs with and without Immune Enhancing Edits (IEEs) in 786-O Model

This study compared the efficacy of T-cells engineered with construct 5718 or construct 5719 (SEQ ID NO: 106) with and without IEE(s) to benchmark construct 4645, in the presence of absence of TGFβ against a 786-O cell line. T-cells were engineered with construct 5718 alone, 5718+CD70 KO, construct 5718+ TGFBR2 KO, construct 5718+DKO, construct 5719 alone, construct 5719+CD70 KO, construct 5719+ TGFBR2 KO, construct 5719+DKO, benchmark construct 4645 alone, or were untreated.

4.1 Engineering T Cells with Construct 5718 or 5719, and Immune Enhancing Edits (IEEs)

T-cells were isolated from peripheral blood of healthy human donor 535 with the following MHC I phenotype: HLA-A*02:01:01G, 03:01:01G, HLA-B*07:02:01G, 15:01:01, HLA-C*03:04:01, 07:02:01G, HLA-DRB1*07:01:01, 15:01:01, HLA-DRB4*01:03:01: 02N, HLA-DRB5*01:01:01, HLA-DQA1*01:02:01, 02:01:01, HLA-DQB1*03:03:02, 06:02:01, HLA-DPA1-01:03:01, 01:03:01 HLA-DPB1*04:01:01, 04:04:01. Briefly, a leukapheresis pack (HemaCare Technologies) was treated in ammonium chloride RBC lysis buffer (Stemcell Technologies; Cat. 07800) for 15 minutes to lyse red blood cells. Peripheral blood mononuclear cell (PBMC) count was determined post lysis and T-cell isolation was performed using EasySep Human T cell isolation kit (Stemcell Technologies, Cat. 17951) according to manufacturer's protocol. Isolated CD3+-T-cells were resuspended in Cryostore CS10 media (Stemcell Technologies, Cat. 07930) and frozen down in liquid nitrogen until further use. T-cells were engineered with IEE edits as in Example 6.1 and cryopreserved for future use.

Frozen T-cells were thawed at a cell concentration of 1×10\hat{ }6 cells/mL into T cell growth media (TCGM). T-cells of each condition were incubated overnight at 37° C.

On the same day that T-cells were thawed, 786-O cells were plated. 25-30 mL of tumor cell media containing GFP-Luciferase 786-O tumor cells, RPMI (Corning, Cat. 10-040-CV), 10% FBS, and 1% P/S were added to a T-75 flask. Cells were mixed and centrifuged at 500 RCF for 5 minutes. Cells were then resuspended in 5-10 mL of tumor cell media and mixed. Cells were counted and then plated at a density of 10,000 cells per 100 μL well in a 96-well plate. The plates were rested overnight in the incubator at 37° C.

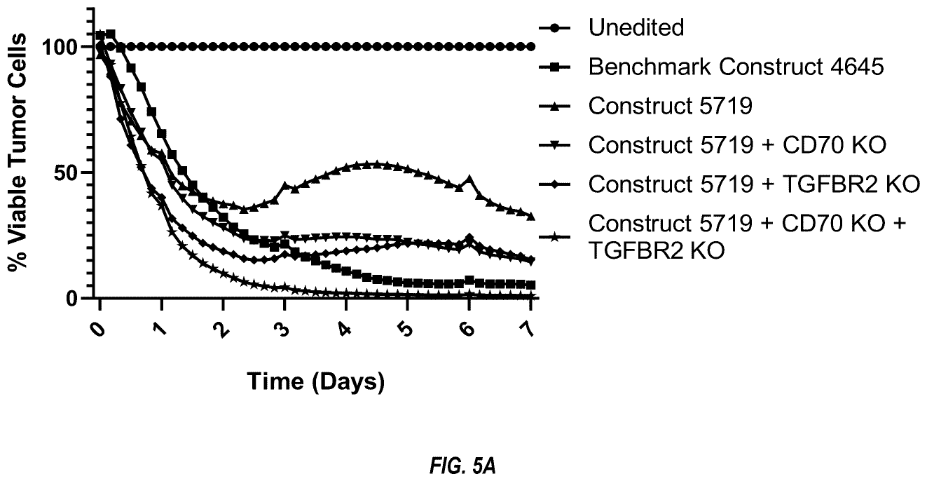

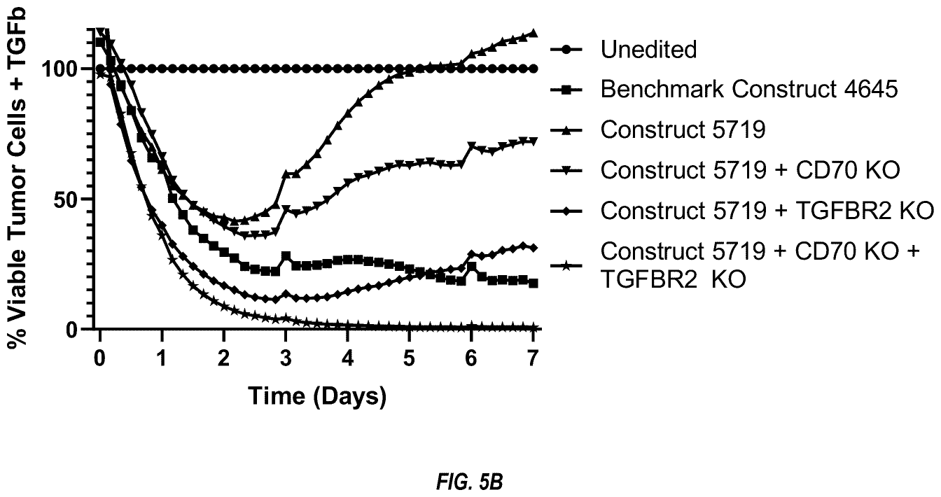

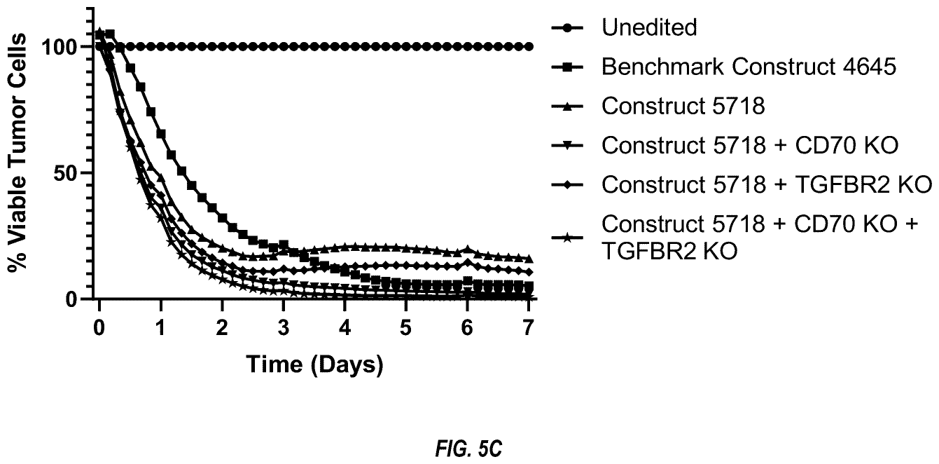

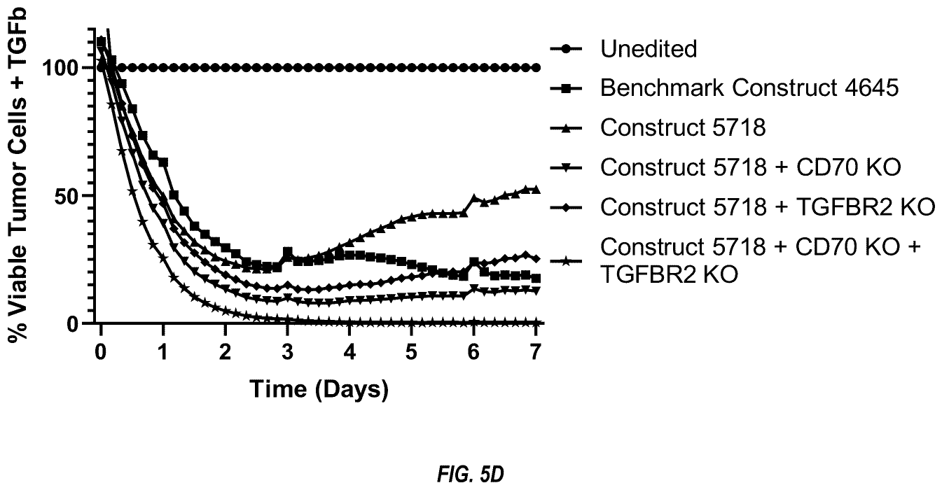

24 hours after thawing, T-cells were removed from the incubator and centrifuged at 300 RCF for 5 minutes. T-cells were normalized to the lowest anti-CD70 CAR expression and the live cell/mL count and were resuspended in tumor cell media in the 96-well plate. 5 μl of soluble TGFβ master mix was added to the wells designated to receive TGFβ based on the experimental plan. Cytotoxicity was measured using Incucyte cell imaging. The results for the 786-O tumor cell line are shown in FIGS. 5A-D.

### Example 5: Re-Challenging Anti-CD70 CAR-T Cells with or without Immune Enhancing Edits (IEEs) with 786-0 or ACHN Tumor Cells

### 5.1 Thawing and Resting CAR-T and Control T-Cells

T-cells were engineered with IEEs as in Example 6.1.

Anti-CD70 CAR-T cells engineered with constructs 5719 (SEQ ID NO: 106), 5281, 5715, or 6115 and further comprising IEEs were assessed for efficacy in a serial re-challenge assay. Additionally, T-cells having TRAC KO only or expressing the benchmark construct 4645 were also engineered for comparison.

On Day 0, cryopreserved anti-CD70 CAR-T cells were thawed in the 37° C. water bath and transferred to a 15 mL conical tube containing 9 mL of pre-warmed T-cell activation media (TCAM). 1 mL of TCGM media was added to vials and transferred to 15 mL conical tubes to obtain residual cell suspension. The 15 mL conical tubes were centrifuged at 300 RCF for 3 minutes at room temperature. After centrifugation, the supernatant was aspirated, and the cell pellet was resuspended in TCGM media with cytokines. An aliquot was obtained for cell counting using the Cellaca instrument. The anti-CD70 CAR-T cells were adjusted at a concentration of 1.0×10\hat{ }6 cells/mL. The T-cells were transferred to T-75 flasks and incubated overnight in a 37° C. incubator.

5.2 Assay Setup with Adherent Target Cells

On Day 0, the 786-O GFP-Luciferase tumor cells and ACHN GFP-Luciferase tumor cells were harvested from T-75 flasks and counted using the Cellaca counting instrument. After cell counting, 250,000 tumor cells were plated per well in a sterile 24-well clear TC-treated flat-bottom plate (Corning, Cat. 354408). The 24 well plates were incubated overnight in a 37° C. incubator to allow tumor cells to adhere to the plate.

### 5.3 Re-Challenge

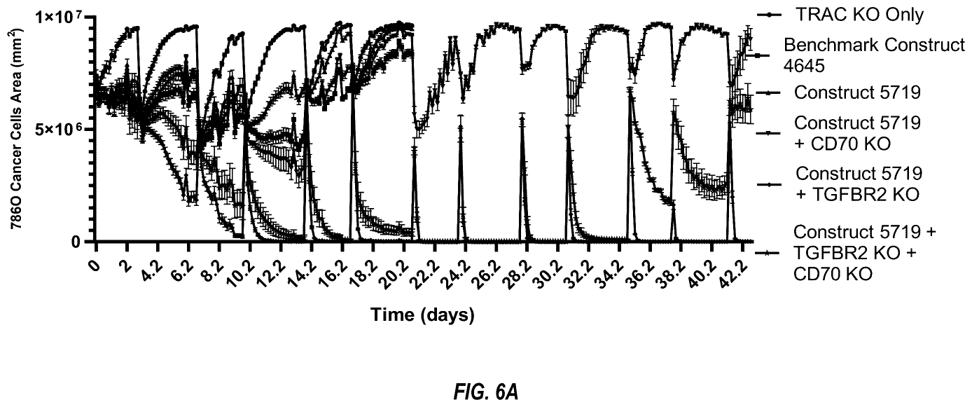

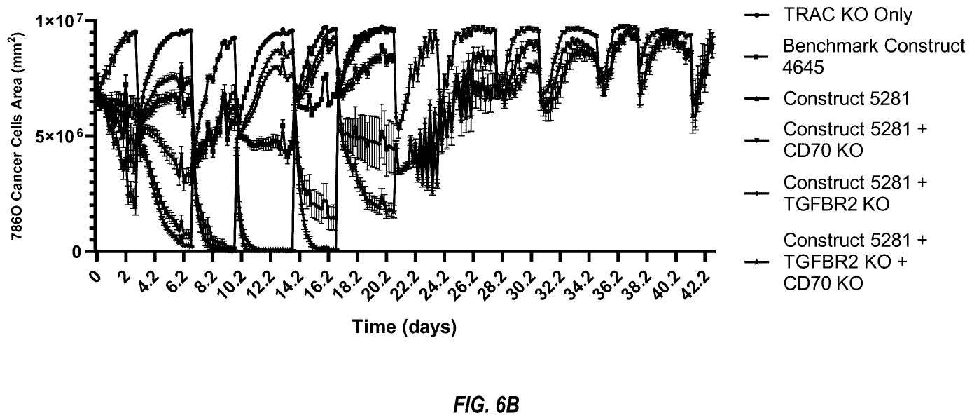

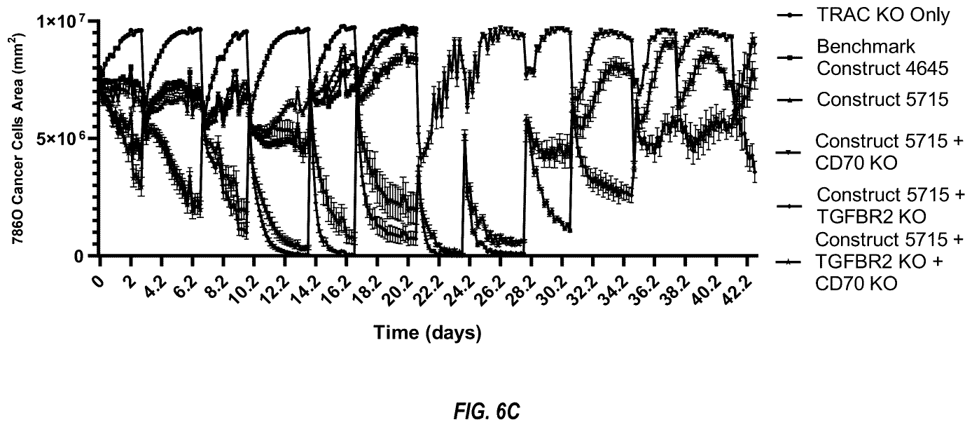

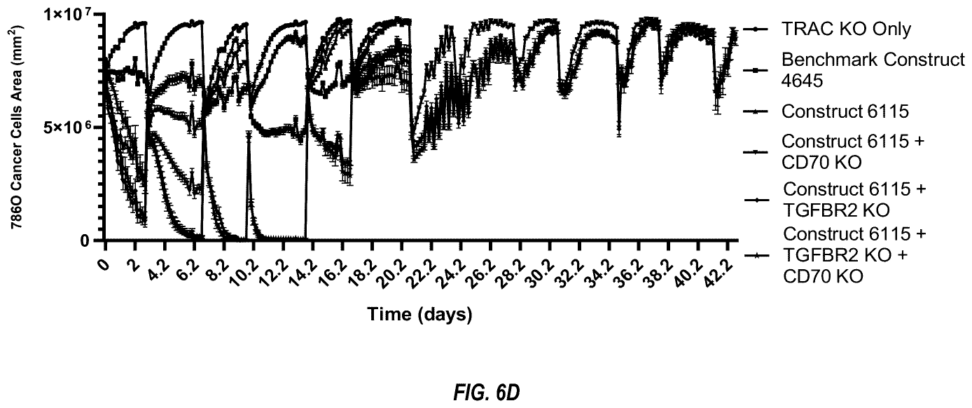

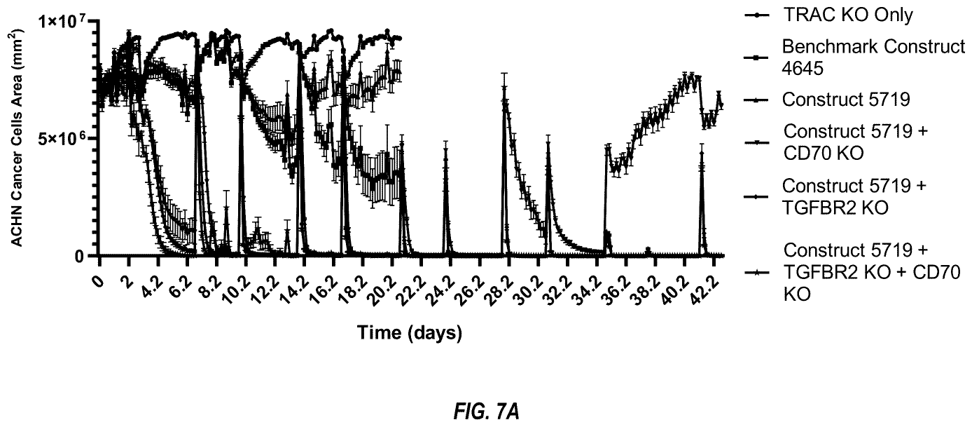

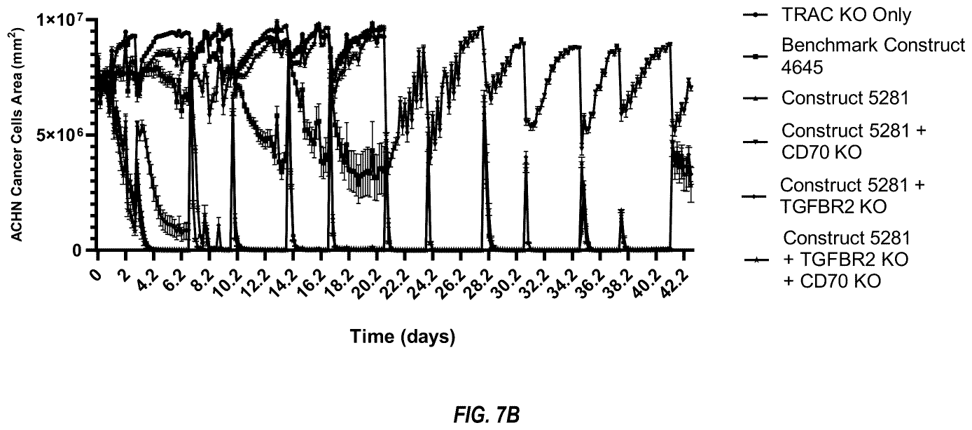

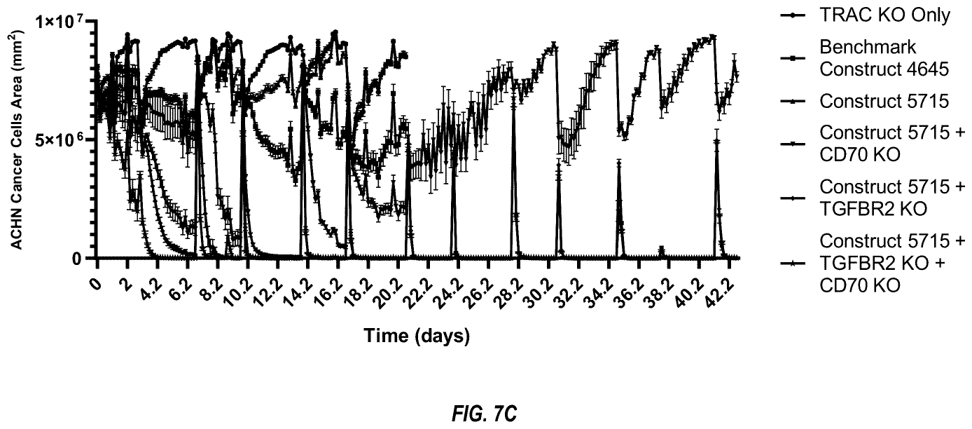

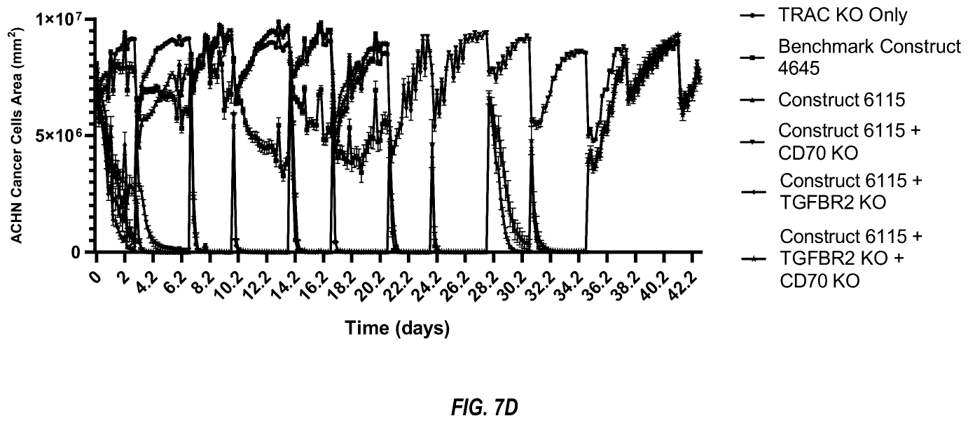

CAR-T cells were cultured with the adhered 786-0 or ACHN GFP-Luciferase tumor cells and rechallenged every 2-4 days. On days of re-challenge, the 24-well plates were removed from the Incucyte instrument and centrifuged at 300 RCF for five minutes. After centrifugation, 1 mL of the supernatant was removed from the 786-O and ACHN plates and the remaining 1 mL was transferred to the 24-well flat-bottom tumor cell plates. Recombinant Human TGFβ (R&D Systems, Cat. 7754-BH-100—CF) was added to each well at a concentration of 50 ng/mL. The 24-well flat-bottom plates were transferred to the Incucyte instrument to monitor cytotoxicity. Results for the re-challenge are shown in FIGS. 6A-6D for the 786-O cell line and FIGS. 7A-7D for the ACHN cell line.

### Example 6—In Vivo Study of Anti-CD70 CAR Constructs with and without Immune Enhancing Edits (IEEs) in a 786-O Model

Female NOG mice were engrafted with 10×10⁶ 786-O-GFP tumor cells followed by the injection of anti-CD70 CAR-T cells engineered with benchmark construct 4645, construct 5715, construct 5719 (SEQ ID NO: 106) or construct 5281 with single or double immune enhancing edits (IEE). IEEs were either CD70 single knockout, TGFBR² single knockout, or CD70 and TGFBR2 double knockouts. Engineered T-cells were injected at a 0.2×10⁶ dose when the solid tumors reached an approximate average volume of 450 mm³. This study compared the efficacy of construct 5715, construct 5719, and construct 5281 with and without IEE(s) in a CD70 antigen^{high} 786-O Renal Cell Carcinoma (RCC) tumor model.

### 6.1 Engineering Anti-CD70 CAR-T Cells

T-cells were isolated from peripheral blood of healthy human donor 535 with the following MHC I phenotype: HLA-A*02:01:01G, 03:01:01G, HLA-B*07:02:01G, 15:01:01, HLA-C*03:04:01, 07:02:01G, HLA-DRB1*07:01:01, 15:01:01, HLA-DRB4*01:01:01, HLA-DQA1*01:02:01, 02:01:01, HLA-DQB1*03:03:02, 06:02:01, HLA-DPA1-01:02:01, HLA-DPB1*04:01:01. Briefly, a leukapheresis pack (HemaCare Technologies) was treated in ammonium chloride RBC lysis buffer (Stemcell Technologies; Cat. 07800) for 15 minutes to lyse red blood cells. Peripheral blood mononuclear cell (PBMC) count was determined post lysis and T cell isolation was performed using EasySep Human T-cell isolation kit (Stemcell Technologies, Cat. 17951) according to manufacturer's protocol. Isolated CD3+ T cells were re-suspended in Cryostore CS10 media (Stemcell Technologies, Cat. 07930) and frozen down in liquid nitrogen until further use.

Frozen T-cells were thawed at a cell concentration of 1×10\hat{ }6 cells/mL into T-cell growth media (TCGM). Cells were rested at 37° C. for 24 hours.

Twenty-four hours post thawing T-cells were counted and resuspended at 1×10\hat{ }6 cells/mL in T-cell activation media (TCAM) and TransACT (Miltenyi) added to a final concentration of 1/100 of the total volume. Cells were mixed and divided into two groups, Wild-type (WT) TGFBR2 competent cells and Knock-out (KO) TGFBR2 cells. For the WT TGFBR2 group, T-cell suspension was incubated at 37° C. for 24 hours. For the KO TGFBR2 group, the T-cell suspension was treated with 2.5 μg/mL ApoE3 (Peprotech, Cat. 350-02) and 0.625 μg/mL of TGFBR2-targeted LNP Guide G029528 and incubated at 37° C. for 48 hours.

48 hours post activation, WT & TGFBR2 KO cells were resuspended at 0.5e6 cells/mL in transduction media. At this point, a fraction of WT cells were aliquoted to become the “Untransduced” sample, resulting in three total T cell conditions (Untransduced, WT & KO TGFBR2). The remainder of the WT cells as well as all of the TGFBR2 KO cells were then transduced with two respective CD70-CAR AAV. Each AAV was removed from-80° C. and thawed on ice. Transduction media was generated from TCAM by adding ApoE3 (Peprotech, Cat. 350-02) to a final concentration of 2.5 μg/mL. Respective AAVs for anti-CD70 CAR constructs were added to WT and TGFBR2 KO cells at an MOI of 3e5 GC/cell. After AAV addition, there were three new groups (Untransduced, WT+anti-CD70 CAR, TGFBR2 KO+anti-CD70 CAR) for each respective CAR construct. All groups were then treated with TRAC-targeted LNP G013006 to a final concentration of 2.5 μg/mL. DNApki Compound 1 was added at a final concentration of 0.25 μM to all of the CAR AAV conditions. The three cell groups (Untransduced, WT+anti-CD70 CAR, TGFBR2 KO+anti-CD70 CAR) for each CAR construct, were then mixed and were incubated at 37° C. for 24 hours.

24 hours post TRAC-LNP and AAV treatment, all conditions (Untransduced, WT+anti-CD70 CAR, TGFBR2 KO+anti-CD70 CAR) for each CAR construct were resuspended to a concentration of 0.5e6 cells/mL in TCAM media. Next, for each CAR construct, the WT+CD70 CAR group was divided into two groups: CAR Alone and CAR+CD70 KO. Then, for each CAR construct, the TGFBR2 KO+anti-CD70 CAR was divided into two groups: CAR+ TGFBR2 KO and CAR+ TGFBR2 KO+CD70 KO (Double KO/DKO). Then, the CAR+CD70 KO and CAR+DKO groups, for each CAR construct, were treated with CD70-targeted LNP (Guide G026733) to a final concentration of 0.625 μg/mL as well as ApoE3 (Peprotech, Cat. 350-02) to a final concentration of 2.5 μg/mL. Cells were incubated at 37° C. for 24 hours.

24 hours post CD70-LNP treatment, all cell conditions for each CAR construct (Untransduced, CAR Alone, CAR+CD70 KO, CAR+ TGFBR2 KO, CAR+DKO) were transferred to GREX plates (Wilson Wolf Cat. 80660M) and expanded for 10 days with regular changes in media and cytokines. After expansion, CAR insertion rates were quantified using flow cytometry, and cells were cryopreserved in Cryostore CS10 freezing media (StemCell Cat. 07930).

6.2 Anti-CD70 CAR Constructs with CD70 and TGFBR2 Double Knockout Induced Complete Tumor Regression in 786-O-GFP Model

For the in vivo efficacy study, 786-O-GFP cells were harvested from culture, washed twice with HBSS (Gibco, Cat. No. 14025-092) and resuspended at 10 e6 cells in 400 μl of HBSS for injections. 50 female NOG mice (Taconic) were dosed subcutaneously on the right flank of the animals. The animals were monitored two times a week for tumor growth by caliper measurements and their tumor volumes were recorded. Once the tumors reached average ~450 mm³ in volume, animals were randomized on day 39 post engraftment followed by T cell infusion. Different groups of anti-CD70 CAR T cells engineered as described above were thawed, washed with HBSS (Gibco, Cat. No. 14025-092) and resuspended at 0.2 e6 in 150 μl of HBSS for injections. Five mice per T-cell group were dosed by tail vein injections in the tumor engrafted animals.

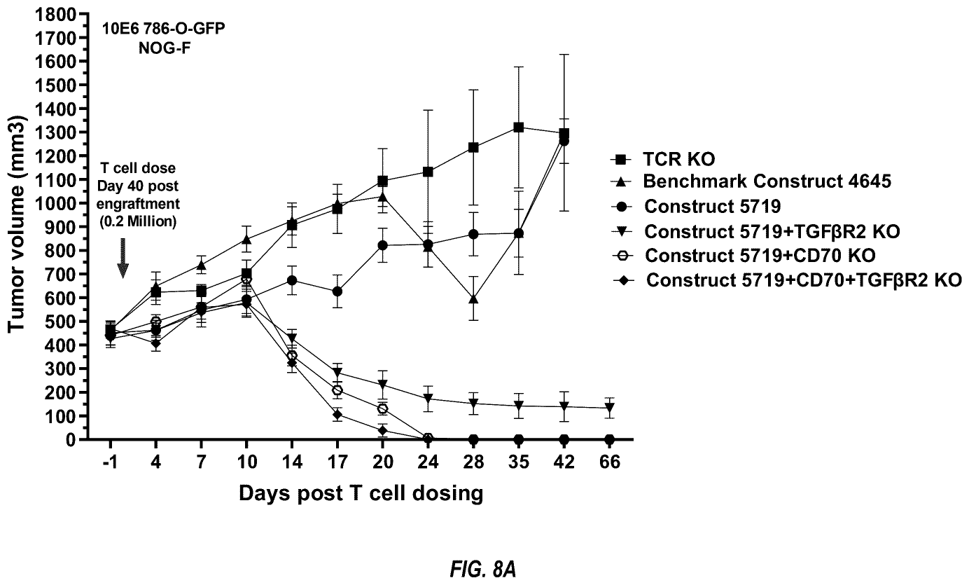

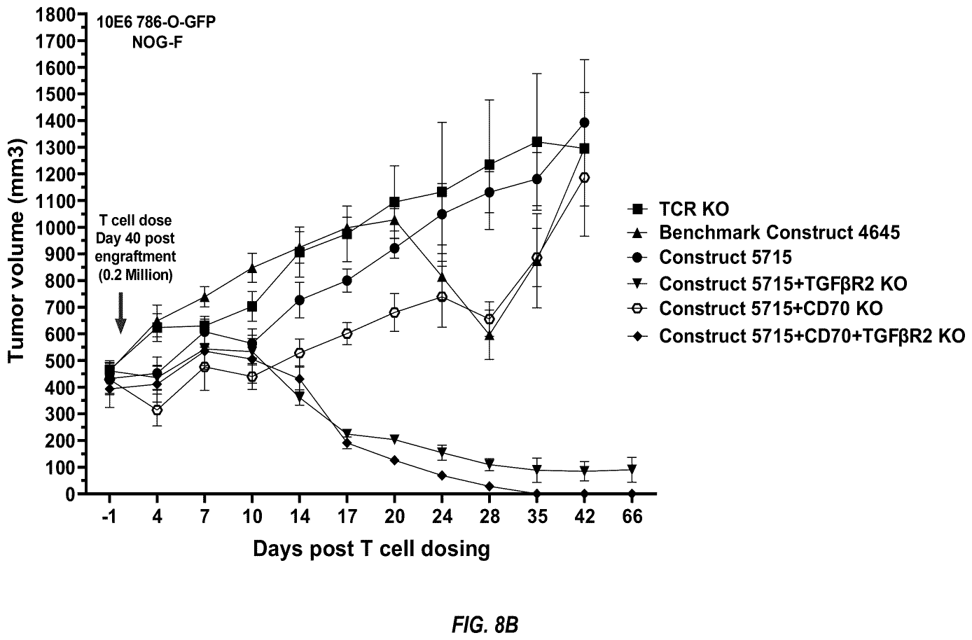

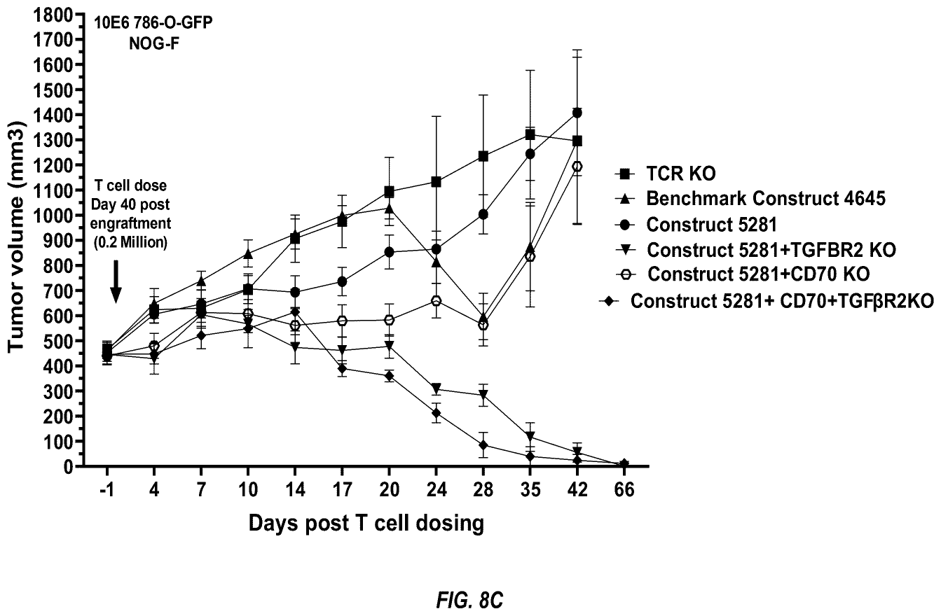

Tumor caliper measurements were done twice or thrice a week post T cell dosing along with recording body weights post T-cell dosing on days-1, 4, 7, 10, 14, 17, 20, 24, 28, 35 and 42. The mice were prepared to be measured by restraining the animal securely and shaving the excess fur off the right-side flank of the animal. The shaved area was then wiped down with an alcohol swab to clearly visualize the tumor and the length and the width of the tumor was measured using calipers. Tumor volumes were calculated as (((Length+Width)/2/2)\hat{ }3)*3.14*1.33. Table 13 and FIGS. 8A-8C show the average tumor volume data for each group dosed with different constructs from day of randomization (Day 39 post engraftment) until study termination. Engineered anti-CD70 CAR-T cells were dosed on day 40 post engraftment.

Similar results were achieved in an ACHN tumor cell model in which double knockout and single knockout constructs improved the efficacy of constructs.

TABLE 13
Average tumor volumes from all groups post T-cell dosing.

| | | | | | |
|---|---|---|---|---|---|
| | Days post | Days post T | Average | SD of | |
| T cell dose | engraftment | cell dosing | TVols | TVols | N |

TCR KO

| | | | | | |
|---|---|---|---|---|---|
| 0.2e6/animal | 39 | −1 | 464.944 | 67.4 | 5 |
| 0.2e6/animal | 44 | 4 | 623.532 | 104.6 | 5 |
| 0.2e6/animal | 47 | 7 | 629.88 | 51.9 | 5 |
| 0.2e6/animal | 50 | 10 | 703.19 | 112.4 | 5 |
| 0.2e6/animal | 54 | 14 | 906.5125 | 163.1 | 4 |
| 0.2e6/animal | 57 | 17 | 973.78 | 181.1 | 4 |
| 0.2e6/animal | 60 | 20 | 1045.315 | 235.2 | 4 |
| 0.2e6/animal | 64 | 24 | 1132.45 | 260.5 | 2 |
| 0.2e6/animal | 68 | 28 | 1234.81 | 243.4 | 2 |
| 0.2e6/animal | 75 | 35 | 1320.475 | 256.2 | 2 |
| 0.2e6/animal | 82 | 42 | 1295.26 | 0.0 | 2 |

Construct 4645

| | | | | | |
|---|---|---|---|---|---|
| 0.2e6/animal | 39 | −1 | 460.918 | 64.2 | 5 |
| 0.2e6/animal | 44 | 4 | 648.75 | 119.3 | 5 |
| 0.2e6/animal | 47 | 7 | 739.174 | 75.6 | 5 |
| 0.2e6/animal | 50 | 10 | 847.646 | 109.0 | 5 |
| 0.2e6/animal | 54 | 14 | 924.66 | 119.0 | 5 |
| 0.2e6/animal | 57 | 17 | 998.082 | 78.5 | 5 |
| 0.2e6/animal | 60 | 20 | 1027.666 | 84.4 | 5 |
| 0.2e6/animal | 64 | 24 | 814.788 | 172.4 | 5 |
| 0.2e6/animal | 68 | 28 | 596.674 | 185.7 | 5 |
| 0.2e6/animal | 75 | 35 | 874.312 | 352.3 | 5 |
| 0.2e6/animal | 82 | 42 | 1297.37 | 661.7 | 5 |

Construct 5281

| | | | | | |
|---|---|---|---|---|---|
| 0.2e6/animal | 39 | −1 | 452.644 | 68.5 | 5 |
| 0.2e6/animal | 44 | 4 | 605.566 | 71.0 | 5 |
| 0.2e6/animal | 47 | 7 | 646.618 | 112.3 | 5 |
| 0.2e6/animal | 50 | 10 | 707.32 | 119.0 | 5 |
| 0.2e6/animal | 54 | 14 | 693.046 | 130.3 | 5 |
| 0.2e6/animal | 57 | 17 | 735.66 | 112.6 | 5 |
| 0.2e6/animal | 60 | 20 | 853.026 | 134.5 | 5 |
| 0.2e6/animal | 64 | 24 | 864.908 | 141.5 | 5 |
| 0.2e6/animal | 68 | 28 | 1003.11 | 155.9 | 5 |
| 0.2e6/animal | 75 | 35 | 1244.218 | 212.4 | 5 |
| 0.2e6/animal | 82 | 42 | 1407.243 | 354.8 | 3 |

Construct 5281 + CD70 KO

| | | | | | |
|---|---|---|---|---|---|
| 0.2e6/animal | 39 | −1 | 439.766 | 69.8 | 5 |
| 0.2e6/animal | 44 | 4 | 479.658 | 100.2 | 5 |
| 0.2e6/animal | 47 | 7 | 612.344 | 111.5 | 5 |
| 0.2e6/animal | 50 | 10 | 607.568 | 112.9 | 5 |
| 0.2e6/animal | 54 | 14 | 561.236 | 144.3 | 5 |
| 0.2e6/animal | 57 | 17 | 579.014 | 127.5 | 5 |
| 0.2e6/animal | 60 | 20 | 582.23 | 128.2 | 5 |
| 0.2e6/animal | 64 | 24 | 659.084 | 138.0 | 5 |
| 0.2e6/animal | 68 | 28 | 563.034 | 168.3 | 5 |
| 0.2e6/animal | 75 | 35 | 835.382 | 402.1 | 5 |
| 0.2e6/animal | 82 | 42 | 1194.048 | 400.1 | 4 |
| 0.2e6/animal | 89 | 66 | 0 | 0.0 | 4 |

Construct 5281 + TGFβR2 KO

| | | | | | |
|---|---|---|---|---|---|
| 0.2e6/animal | 39 | −1 | 445.474 | 82.6 | 5 |
| 0.2e6/animal | 44 | 4 | 428.682 | 124.7 | 5 |
| 0.2e6/animal | 47 | 7 | 603.332 | 106.9 | 5 |
| 0.2e6/animal | 50 | 10 | 566.38 | 68.6 | 5 |
| 0.2e6/animal | 54 | 14 | 472.9875 | 113.8 | 5 |
| 0.2e6/animal | 57 | 17 | 461.2625 | 92.3 | 5 |
| 0.2e6/animal | 60 | 20 | 477.455 | 80.5 | 5 |
| 0.2e6/animal | 64 | 24 | 306.0575 | 40.5 | 5 |
| 0.2e6/animal | 68 | 28 | 282.545 | 75.9 | 5 |
| 0.2e6/animal | 75 | 35 | 116.8425 | 98.9 | 5 |
| 0.2e6/animal | 82 | 42 | 55.2875 | 66.7 | 5 |
| 0.2e6/animal | 89 | 66 | 0 | 0.0 | 5 |

Construct 5281 + CD70 + TGFβR2 KO

| | | | | | |
|---|---|---|---|---|---|
| 0.2e6/animal | 39 | −1 | 446.406 | 78.3 | 5 |
| 0.2e6/animal | 44 | 4 | 447.494 | 81.0 | 5 |
| 0.2e6/animal | 47 | 7 | 520.6 | 105.2 | 5 |
| 0.2e6/animal | 50 | 10 | 548.58 | 152.3 | 5 |
| 0.2e6/animal | 54 | 14 | 614.538 | 181.3 | 5 |
| 0.2e6/animal | 57 | 17 | 389.108 | 64.2 | 5 |
| 0.2e6/animal | 60 | 20 | 359.82 | 47.3 | 5 |
| 0.2e6/animal | 64 | 24 | 212.6 | 68.1 | 5 |
| 0.2e6/animal | 68 | 28 | 84.67 | 87.2 | 5 |
| 0.2e6/animal | 75 | 35 | 39.605 | 68.6 | 5 |
| 0.2e6/animal | 82 | 42 | 23.8525 | 41.3 | 5 |
| 0.2e6/animal | 89 | 66 | 11.695 | 20.3 | 5 |

Construct 5719

| | | | | | |
|---|---|---|---|---|---|
| 0.2e6/animal | 39 | −1 | 451.5 | 101.1 | 5 |
| 0.2e6/animal | 44 | 4 | 462.21 | 60.5 | 5 |
| 0.2e6/animal | 47 | 7 | 547.222 | 141.8 | 5 |
| 0.2e6/animal | 50 | 10 | 592.448 | 120.2 | 5 |
| 0.2e6/animal | 54 | 14 | 672.778 | 120.5 | 5 |
| 0.2e6/animal | 57 | 17 | 626.798 | 138.5 | 5 |
| 0.2e6/animal | 60 | 20 | 821.624 | 144.8 | 5 |
| 0.2e6/animal | 64 | 24 | 825.55 | 191.3 | 5 |
| 0.2e6/animal | 68 | 28 | 868.818 | 182.9 | 5 |
| 0.2e6/animal | 75 | 35 | 872.92 | 203.1 | 5 |
| 0.2e6/animal | 82 | 42 | 1262.328 | 164.2 | 4 |

Construct 5719 + CD70 KO

| | | | | | |
|---|---|---|---|---|---|
| 0.2e6/animal | 39 | −1 | 441.224 | 83.0 | 5 |
| 0.2e6/animal | 44 | 4 | 499.094 | 58.4 | 5 |
| 0.2e6/animal | 47 | 7 | 560.73 | 34.5 | 5 |
| 0.2e6/animal | 50 | 10 | 678.674 | 64.7 | 5 |
| 0.2e6/animal | 54 | 14 | 355.862 | 86.9 | 5 |
| 0.2e6/animal | 57 | 17 | 208.56 | 73.1 | 5 |
| 0.2e6/animal | 60 | 20 | 130.536 | 54.1 | 5 |
| 0.2e6/animal | 64 | 24 | 7.436 | 14.9 | 5 |
| 0.2e6/animal | 68 | 28 | 0 | 0.0 | 5 |
| 0.2e6/animal | 75 | 35 | 0 | 0.0 | 5 |
| 0.2e6/animal | 82 | 42 | 0 | 0.0 | 5 |
| 0.2e6/animal | 89 | 49 | 0 | 0.0 | 5 |

Construct 5719 + TGFβR2 KO

| | | | | | |
|---|---|---|---|---|---|
| 0.2e6/animal | 39 | −1 | 427.312 | 76.2 | 5 |
| 0.2e6/animal | 44 | 4 | 461.89 | 84.8 | 5 |
| 0.2e6/animal | 47 | 7 | 536.398 | 84.0 | 5 |
| 0.2e6/animal | 50 | 10 | 579.326 | 114.6 | 5 |
| 0.2e6/animal | 54 | 14 | 426.404 | 78.9 | 5 |
| 0.2e6/animal | 57 | 17 | 281.996 | 79.6 | 5 |
| 0.2e6/animal | 60 | 20 | 231.386 | 120.2 | 5 |
| 0.2e6/animal | 64 | 24 | 171.99 | 109.1 | 5 |
| 0.2e6/animal | 68 | 28 | 152.208 | 93.0 | 5 |
| 0.2e6/animal | 75 | 35 | 142.5 | 105.0 | 5 |
| 0.2e6/animal | 82 | 42 | 138.688 | 127.2 | 5 |
| 0.2e6/animal | 89 | 49 | 133.1 | 86.7 | 5 |

Construct 5719 + CD70 + TGFβR2 KO

| | | | | | |
|---|---|---|---|---|---|
| 0.2e6/animal | 39 | −1 | 469.666 | 64.3 | 5 |
| 0.2e6/animal | 44 | 4 | 406.988 | 66.5 | 5 |
| 0.2e6/animal | 47 | 7 | 559.118 | 98.9 | 5 |
| 0.2e6/animal | 50 | 10 | 571.902 | 110.2 | 5 |
| 0.2e6/animal | 54 | 14 | 324.53 | 81.7 | 5 |
| 0.2e6/animal | 57 | 17 | 106.164 | 57.6 | 5 |
| 0.2e6/animal | 60 | 20 | 38.442 | 55.8 | 5 |
| 0.2e6/animal | 64 | 24 | 0 | 0.0 | 5 |
| 0.2e6/animal | 68 | 28 | 0 | 0.0 | 5 |
| 0.2e6/animal | 75 | 35 | 0 | 0.0 | 5 |
| 0.2e6/animal | 82 | 42 | 0 | 0.0 | 5 |
| 0.2e6/animal | 89 | 49 | 0 | 0.0 | 5 |

Construct 5715

| | | | | | |
|---|---|---|---|---|---|
| 0.2e6/animal | 39 | −1 | 432.342 | 120.9 | 5 |
| 0.2e6/animal | 44 | 4 | 451.832 | 122.4 | 5 |
| 0.2e6/animal | 47 | 7 | 607.96 | 115.5 | 5 |
| 0.2e6/animal | 50 | 10 | 563.858 | 110.3 | 5 |
| 0.2e6/animal | 54 | 14 | 726.436 | 134.0 | 5 |
| 0.2e6/animal | 57 | 17 | 799.844 | 86.8 | 5 |
| 0.2e6/animal | 60 | 20 | 920.996 | 73.6 | 5 |
| 0.2e6/animal | 64 | 24 | 1048.904 | 230.6 | 5 |
| 0.2e6/animal | 68 | 28 | 1130.996 | 153.3 | 5 |
| 0.2e6/animal | 75 | 35 | 1180.606 | 199.7 | 5 |
| 0.2e6/animal | 82 | 42 | 1317.903 | 189.3 | 5 |

Construct 5715 + CD70 KO

| | | | | | |
|---|---|---|---|---|---|
| 0.2e6/animal | 39 | −1 | 429.164 | 104.0 | 5 |
| 0.2e6/animal | 44 | 4 | 314.368 | 118.9 | 5 |
| 0.2e6/animal | 47 | 7 | 476.24 | 176.2 | 5 |
| 0.2e6/animal | 50 | 10 | 439.908 | 98.4 | 5 |
| 0.2e6/animal | 54 | 14 | 527.856 | 105.9 | 5 |
| 0.2e6/animal | 57 | 17 | 600.744 | 84.2 | 5 |
| 0.2e6/animal | 60 | 20 | 680.19 | 142.0 | 5 |
| 0.2e6/animal | 64 | 24 | 739.242 | 228.7 | 5 |
| 0.2e6/animal | 68 | 28 | 655.466 | 129.5 | 5 |
| 0.2e6/animal | 75 | 35 | 886.608 | 218.7 | 5 |
| 0.2e6/animal | 82 | 42 | 1186.106 | 213.3 | 5 |
| 0.2e6/animal | 89 | 49 | | | 5 |

Construct 5715 + TGFβR2 KO

| | | | | | |
|---|---|---|---|---|---|
| 0.2e6/animal | 39 | −1 | 460.578 | 58.1 | 5 |
| 0.2e6/animal | 44 | 4 | 435.178 | 92.8 | 5 |
| 0.2e6/animal | 47 | 7 | 542.946 | 125.5 | 5 |
| 0.2e6/animal | 50 | 10 | 532.844 | 99.8 | 5 |
| 0.2e6/animal | 54 | 14 | 360.896 | 57.8 | 5 |
| 0.2e6/animal | 57 | 17 | 224.066 | 26.2 | 5 |
| 0.2e6/animal | 60 | 20 | 202.964 | 31.2 | 5 |
| 0.2e6/animal | 64 | 24 | 154.35 | 57.9 | 5 |
| 0.2e6/animal | 68 | 28 | 109.38 | 45.2 | 5 |
| 0.2e6/animal | 75 | 35 | 88.184 | 91.7 | 5 |
| 0.2e6/animal | 82 | 42 | 84.43 | 63.5 | 4 |
| 0.2e6/animal | 89 | 49 | 90.6875 | 80.3 | 4 |

Construct 5715 + CD70 + TGFβR2 KO

| | | | | | |
|---|---|---|---|---|---|
| 0.2e6/animal | 39 | −1 | 393.58 | 139.6 | 5 |
| 0.2e6/animal | 44 | 4 | 411.014 | 135.1 | 5 |
| 0.2e6/animal | 47 | 7 | 534.782 | 134.9 | 5 |
| 0.2e6/animal | 50 | 10 | 505.372 | 179.9 | 5 |
| 0.2e6/animal | 54 | 14 | 430.768 | 98.2 | 5 |
| 0.2e6/animal | 57 | 17 | 191.506 | 45.5 | 5 |
| 0.2e6/animal | 60 | 20 | 125.598 | 16.3 | 5 |
| 0.2e6/animal | 64 | 24 | 68.838 | 36.9 | 5 |
| 0.2e6/animal | 68 | 28 | 27.924 | 25.0 | 5 |
| 0.2e6/animal | 75 | 35 | 0 | 0.0 | 5 |
| 0.2e6/animal | 82 | 42 | 0 | 0.0 | 5 |
| 0.2e6/animal | 89 | 49 | 0 | 0.0 | 5 |

6.3 Anti-CD70 CAR Constructs 5719 and 5715 with CD70 and TGFBR2 Double Knockout Induced Complete Tumor Clearance Upon Tumor Re-Challenge In Vivo in 786-O-GFP Model

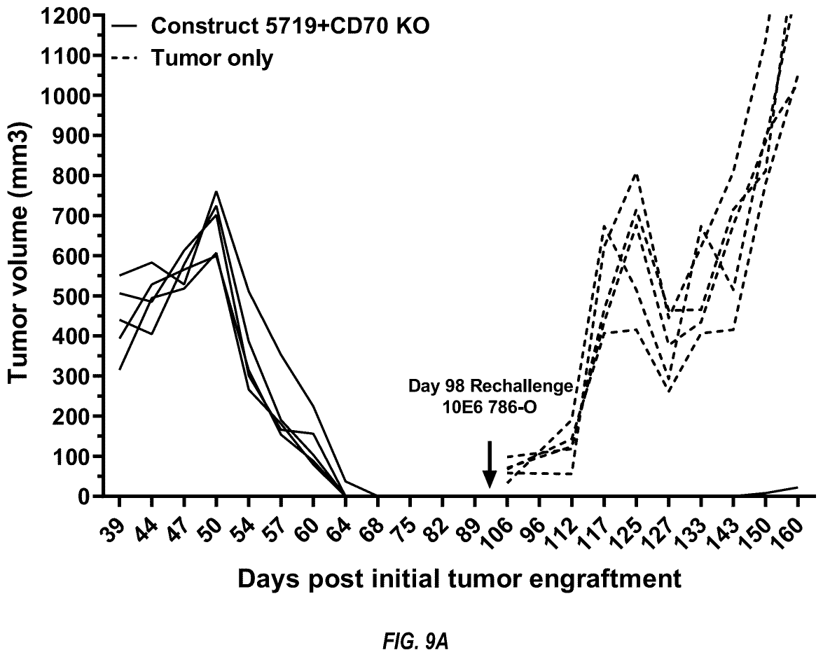

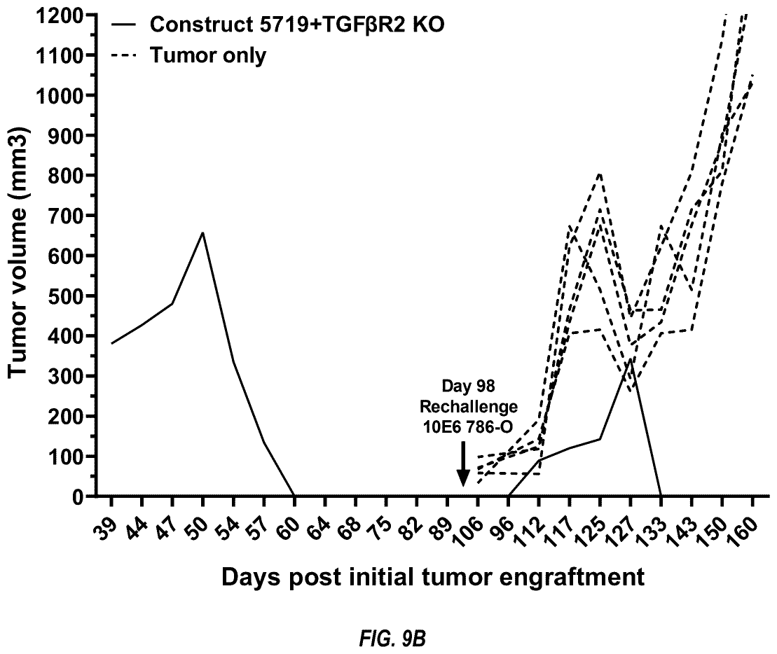

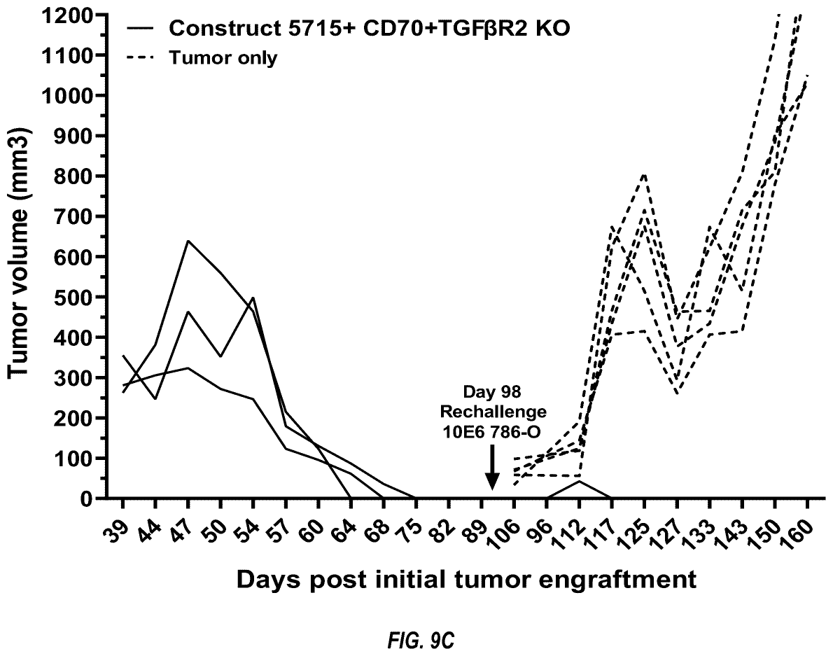

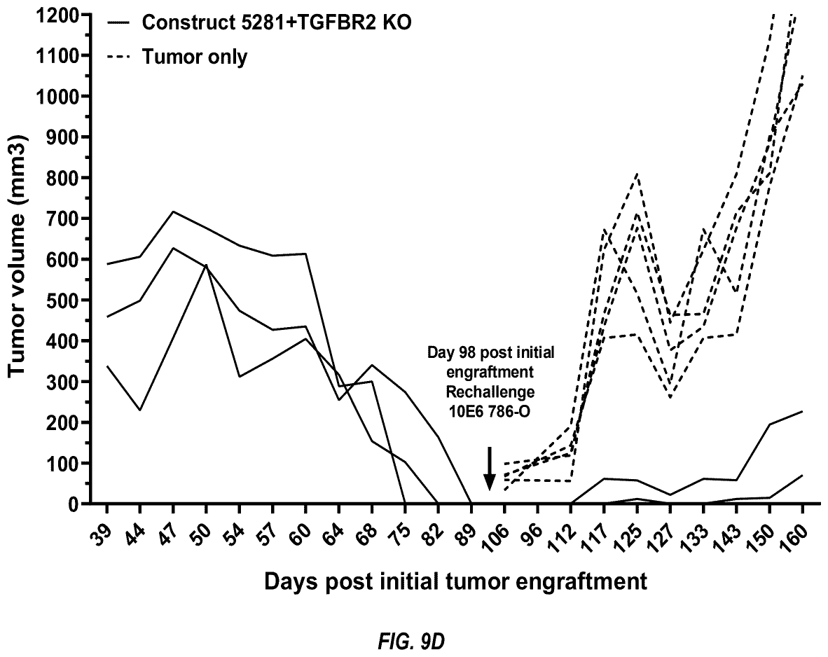

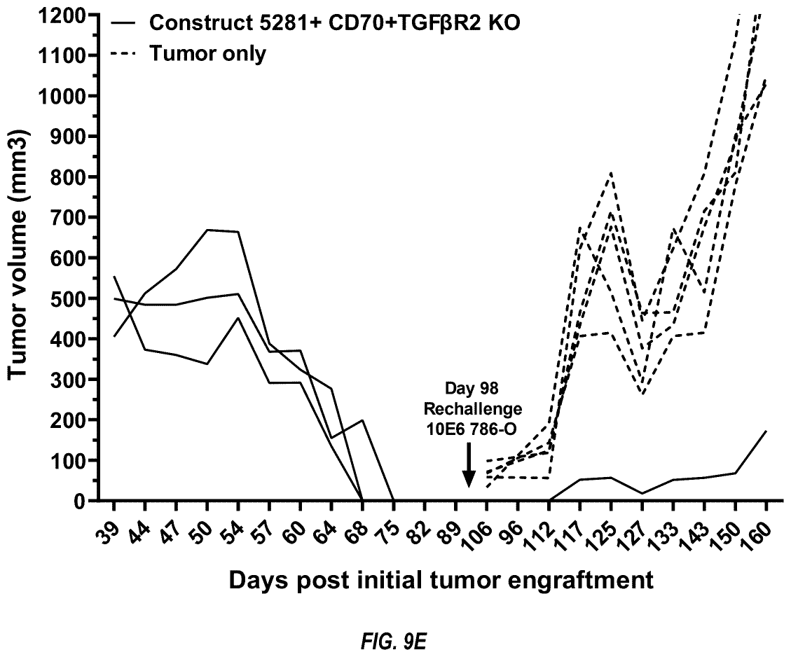

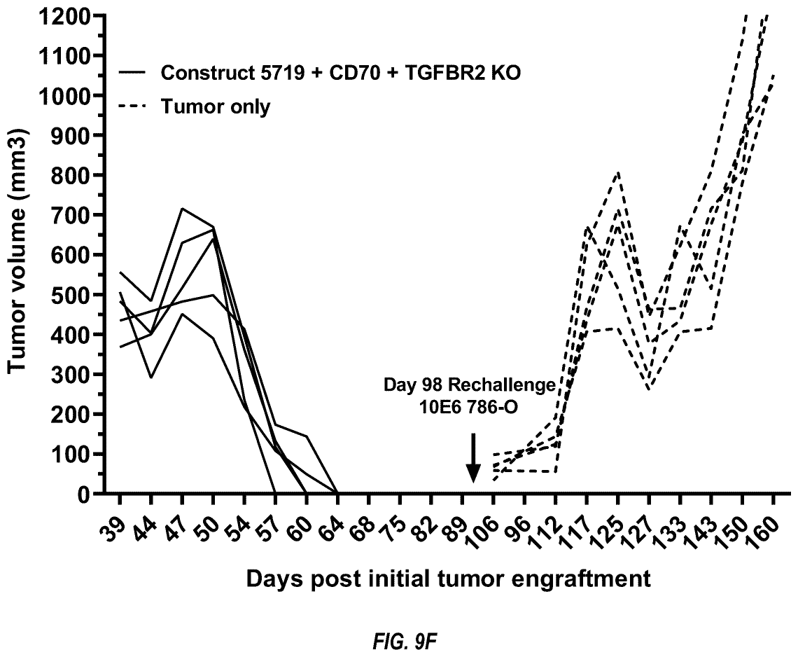

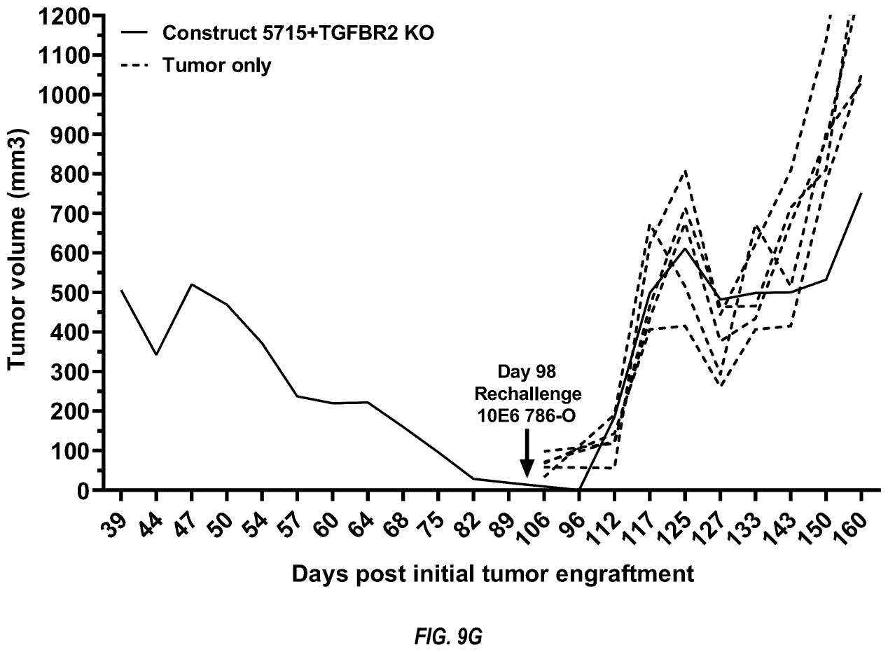

For the in vivo re-challenge study, 786-O-GFP cells were harvested from culture, washed twice with HBSS (Gibco, Cat. No. 14025-092) and resuspended at 10×10⁶ cells in 400 μl of HBSS for injections. All the animals that had complete tumor clearance were engrafted subcutaneously on their right flank with 786-O-GFP cells on day 98 post initial tumor engraftment. Additional five female NOG mice were engrafted with 10×10⁶ 786-O-GFP cells as a ‘tumor only control’ for the re-challenge experiment. The animals were monitored 2-3 times a week on days 14, 19, 27, 29, 35, 45, 52, 62 post re-challenge until study termination (day 72 post re-challenge) for tumor growth by caliper measurements and their tumor volumes were recorded. The average tumor volumes and animals re-challenged are shown in Table 14 and FIGS. 9A-9G.

The rationale for performing an in vivo tumor re-challenge experiment was to assess the persistence of anti-CD70 CAR-T cells with IEE(s), and to further evaluate CD70 and TGFBR2 as a single or double IEEs. All five animals in construct 5719+CD70+ TGFBR2 DKO and construct 5715+CD70+ TGFBR2 DKO treated groups did not exhibit tumor formation post tumor re-challenge. In comparison, redosing tumor cells induced tumor formation in tumor control group, as well as in certain anti-CD70 CAR-T cell+IEE(s) treated groups.

TABLE 14
Average tumor volumes from all groups post tumor re-challenge.

| | | | | | | |
|---|---|---|---|---|---|---|
| Tumor re- | Days post | | Days post | | | |
| challenge cell | initial tumor | Days post | T cell | Average | SD of | |
| dose | engraftment | re-challenge | dosing | TVols | TVols | N |

Tumor only

| | | | | | | |
|---|---|---|---|---|---|---|
| 10e6/animal | N/A | 14 | 72 | 126.604 | 43.5 | 5 |
| 10e6/animal | N/A | 19 | 77 | 520.422 | 107.0 | 5 |
| 10e6/animal | N/A | 27 | 85 | 299.152 | 50.4 | 5 |
| 10e6/animal | N/A | 29 | 87 | 367.85 | 80.0 | 5 |
| 10e6/animal | N/A | 35 | 93 | 520.422 | 107.0 | 5 |
| 10e6/animal | N/A | 45 | 103 | 625.928 | 141.8 | 5 |
| 10e6/animal | N/A | 52 | 110 | 902.798 | 125.9 | 5 |
| 10e6/animal | N/A | 62 | 120 | 1282.576 | 229.2 | 5 |

Construct 5719 + CD70 KO

| | | | | | | |
|---|---|---|---|---|---|---|
| 10e6/animal | 96 | −2 | 56 | 0 | 0.0 | 5 |
| 10e6/animal | 112 | 14 | 72 | 0 | 0.0 | 5 |
| 10e6/animal | 117 | 19 | 77 | 0 | 0.0 | 5 |
| 10e6/animal | 125 | 27 | 85 | 0 | 0.0 | 5 |
| 10e6/animal | 127 | 29 | 87 | 0 | 0.0 | 5 |
| 10e6/animal | 133 | 35 | 93 | 0 | 0.0 | 5 |
| 10e6/animal | 143 | 45 | 103 | 0 | 0.0 | 5 |
| 10e6/animal | 150 | 52 | 110 | 1.632 | 3.3 | 5 |
| 10e6/animal | 160 | 62 | 120 | 4.476 | 9.0 | 5 |

Construct 5719 + TGFβR2 KO

| | | | | | | |
|---|---|---|---|---|---|---|
| 10e6/animal | 96 | −2 | 56 | 50 | 50.0 | 2 |
| 10e6/animal | 112 | 14 | 72 | 82.27 | 6.7 | 2 |
| 10e6/animal | 117 | 19 | 77 | 57.55 | 0.0 | 1 |
| 10e6/animal | 125 | 27 | 85 | 69.25 | 0.0 | 1 |
| 10e6/animal | 127 | 29 | 87 | 27.97 | 0.0 | 1 |
| 10e6/animal | 133 | 35 | 93 | 57.55 | 0.0 | 1 |
| 10e6/animal | 143 | 45 | 103 | 142.37 | 0.0 | 1 |
| 10e6/animal | 150 | 52 | 110 | 152.09 | 0.0 | 1 |
| 10e6/animal | 160 | 62 | 120 | 175 | 0.0 | 1 |

Construct 5719 + CD70 + TGFβR2 KO

| 10e6/animal | 96 | −2 | 56 | 0 | 0.0 | 5 |
|---|---|---|---|---|---|---|
| 10e6/animal | 112 | 14 | 72 | 0 | 0.0 | 5 |
| 10e6/animal | 117 | 19 | 77 | 0 | 0.0 | 5 |
| 10e6/animal | 125 | 27 | 85 | 0 | 0.0 | 5 |
| 10e6/animal | 127 | 29 | 87 | 0 | 0.0 | 5 |
| 10e6/animal | 133 | 35 | 93 | 0 | 0.0 | 5 |
| 10e6/animal | 143 | 45 | 103 | 0 | 0.0 | 5 |
| 10e6/animal | 150 | 52 | 110 | 0 | 0.0 | 5 |
| 10e6/animal | 160 | 62 | 120 | 0 | 0.0 | 5 |
| Construct 5715 + TGFβR2 KO | | | | | | |
| 10e6/animal | 96 | −2 | 56 | 0 | 0.0 | 1 |
| 10e6/animal | 112 | 14 | 72 | 185.26 | 0.0 | 1 |
| 10e6/animal | 117 | 19 | 77 | 498.88 | 0.0 | 1 |
| 10e6/animal | 125 | 27 | 85 | 194.44 | 0.0 | 1 |
| 10e6/animal | 127 | 29 | 87 | 482.36 | 0.0 | 1 |
| 10e6/animal | 133 | 35 | 93 | 498.88 | 0.0 | 1 |
| 10e6/animal | 143 | 45 | 103 | 500.12 | 0.0 | 1 |
| 10e6/animal | 150 | 52 | 110 | 532 | 0.0 | 1 |
| 10e6/animal | 160 | 62 | 120 | 752 | 0.0 | 1 |

Construct 5715 + CD70 + TGFβ2 KO

| | | | | | | |
|---|---|---|---|---|---|---|
| 10e6/animal | 96 | −2 | 56 | 0 | 0.0 | 3 |
| 10e6/animal | 112 | 14 | 72 | 0 | 0.0 | 3 |
| 10e6/animal | 117 | 19 | 77 | 0 | 0.0 | 3 |
| 10e6/animal | 125 | 27 | 85 | 0 | 0.0 | 3 |
| 10e6/animal | 127 | 29 | 87 | 0 | 0.0 | 3 |
| 10e6/animal | 133 | 35 | 93 | 0 | 0.0 | 3 |
| 10e6/animal | 143 | 45 | 103 | 0 | 0.0 | 3 |
| 10e6/animal | 150 | 52 | 110 | 0 | 0.0 | 3 |
| 10e6/animal | 160 | 62 | 120 | 0 | 0.0 | 3 |

Construct 5281 + TCFβR2 KO

| | | | | | | |
|---|---|---|---|---|---|---|
| 10e6/animal | 96 | −2 | 56 | 0 | 0.0 | 3 |
| 10e6/animal | 112 | 14 | 72 | 0 | 0.0 | 3 |
| 10e6/animal | 117 | 19 | 77 | 20.41 | 28.9 | 3 |
| 10e6/animal | 125 | 27 | 85 | 10.125 | 0.0 | 2 |
| 10e6/animal | 127 | 29 | 87 | 10.86 | 0.0 | 2 |
| 10e6/animal | 133 | 35 | 93 | 30.615 | 0.0 | 2 |
| 10e6/animal | 143 | 45 | 103 | 34.81 | 0.0 | 2 |
| 10e6/animal | 150 | 52 | 110 | 104.48 | 0.0 | 2 |
| 10e6/animal | 160 | 62 | 120 | 148.585 | 0.0 | 2 |

Construct 5281 + CD70 + TGFβR2 KO

| | | | | | | |
|---|---|---|---|---|---|---|
| 10e6/animal | 96 | −2 | 56 | 0 | 0.0 | 3 |
| 10e6/animal | 112 | 14 | 72 | 0 | 0.0 | 3 |
| 10e6/animal | 117 | 19 | 77 | 17.28 | 24.4 | 3 |
| 10e6/animal | 125 | 27 | 85 | 0 | 0.0 | 3 |
| 10e6/animal | 127 | 29 | 87 | 5.973333 | 8.4 | 3 |
| 10e6/animal | 133 | 35 | 93 | 17.21333 | 24.3 | 3 |
| 10e6/animal | 143 | 45 | 103 | 18.94333 | 26.8 | 3 |
| 10e6/animal | 150 | 52 | 110 | 22.66667 | 32.1 | 3 |
| 10e6/animal | 160 | 62 | 120 | 57.66333 | 81.5 | 3 |

Construct 5719 + CD70 + TGFβR2 KO

| | | | | | | |
|---|---|---|---|---|---|---|
| 10e6/animal | 96 | −2 | 56 | 0 | 0 | 5 |
| 10e6/animal | 112 | 14 | 72 | 0 | 0 | 5 |
| 10e6/animal | 117 | 19 | 77 | 0 | 0 | 5 |
| 10e6/animal | 125 | 27 | 85 | 0 | 0 | 5 |
| 10e6/animal | 127 | 29 | 87 | 0 | 0 | 5 |
| 10e6/animal | 133 | 35 | 93 | 0 | 0 | 5 |
| 10e6/animal | 143 | 45 | 103 | 0 | 0 | 5 |
| 10e6/animal | 150 | 52 | 110 | 0 | 0 | 5 |
| 10e6/animal | 160 | 62 | 120 | 0 | 0 | 5 |

Construct 5715 + TGFBβ2 KO

| | | | | | | |
|---|---|---|---|---|---|---|
| 10e6/animal | 96 | −2 | 56 | 0 | 0 | 1 |
| 10e6/animal | 112 | 14 | 72 | 185.26 | 0 | 1 |
| 10e6/animal | 117 | 19 | 77 | 498.88 | 0 | 1 |
| 10e6/animal | 125 | 27 | 85 | 194.44 | 0 | 1 |
| 10e6/animal | 127 | 29 | 87 | 482.36 | 0 | 1 |
| 10e6/animal | 133 | 35 | 93 | 498.88 | 0 | 1 |
| 10e6/animal | 143 | 45 | 103 | 500.12 | 0 | 1 |
| 10e6/animal | 150 | 52 | 110 | 532 | 0 | 1 |
| 10e6/animal | 160 | 62 | 120 | 752 | 0 | 1 |

### Example 7: Multi-Editing Allo-CD70 CAR-T Cells with LNP Delivery

Allogeneic anti-CD70 CAR-T cells were engineered to achieve efficient multi-edit knockout of HLA-A, HLA-B, CIITA, CD70, TGFBR2, and TRAC using LNP delivery of editing components. Additionally, insertion of an anti-CD70 CAR into the TRAC locus is achieved by transduction of a homology directed repair template delivered with an AAV.

### 7.1. T Cell Preparation

T cells were isolated and cryopreserved by methods known in the art. The day before initiating T cell editing (Day-0), CD4 and CD8 T cells were thawed, combined in a 1:1 ratio, and rested overnight in T cell activation media (TCAM): CTS OpTmizer (Thermofisher, Cat. A3705001) supplemented with 2.5% human AB serum (Valley Biomedical HP1022HI), 1× GlutaMAX (ThermoFisher 35050061), 10 mM HEPES (ThermoFisher 15630080), 100 U/mL Penstrep (Gibco 15140-122), 200 U/mL IL-2 (Peprotech 200-02), IL-7 (Peprotech 200-07), IL-15 (Peprotech, 200-15). Biological replicates were performed using isolated T cells from 3 donors.

### 7.2. LNP Treatment and Expansion of T Cells

On day 1, T cells were harvested and resuspended at a density of 1×10\hat{ }6 cells/mL in TCAM with a 1:100 dilution of TransAct (Miltenyi, 130-111-160). Cells were treated with LNP and AAV as described in Table 15. Treatments included 10 μg/ml ApoE3 (Peprotech, Cat. 350-02) on Day 1 and Day 3 and DNA protein kinase inhibitor (DNApki “Compound 4” as described in published application WO2022221696, referenced herein as “Compound 1”) on Day 3. LNPs were generally prepared as described in Example 1. LNPs were made with a lipid amine to RNA phosphate (N: P) molar ratio of about 6. LNPs with coformulation of gRNA and mRNA used a ratio of gRNA to mRNA of 1:1 by weight. Lipid nanoparticles in this example were prepared with molar ratios of 35 Lipid A: 47.5 cholesterol: 15 DSPC: 2.5 PEG2k-DMG. LNP doses are reported as the mass of total RNA cargo per volume. Cells were incubated at 37C through to Day 4.

TABLE 15
Editing Schedule

| Day | Type | Contents | Dose | |
|---|---|---|---|---|
| Day 1 | LNP | UGI mRNA | 0.15 | ug/ml |
| | LNP | G034983 TGFBR2 (SEQ ID NO: 342), | 1.5 | ug/ml |
| | | base editor mRNA (comprising SEQ ID | | |
| | | NO: 804) | | |
| | LNP | G034208 HLA-B (SEQ ID NO: 452), | 3 | ug/ml |
| | | base editor mRNA (comprising SEQ ID | | |
| | | NO: 804) | | |
| Day 3 | LNP | UGI mRNA | 0.20 | ug/ml |
| | LNP | G034202 HLA-A (SEQ ID NO: 446), | 2.0 | ug/ml |
| | | base editor mRNA (comprising SEQ ID | | |
| | | NO: 804) | | |
| | LNP | G034619 CIITA (SEQ ID NO: 444), | 2.5 | ug/ml |
| | | base editor mRNA (comprising SEQ ID | | |
| | | NO: 804) | | |
| | LNP | G034199 CD70 (SEQ ID NO: 354), | 0.5 | ug/ml |
| | | base editor mRNA (comprising SEQ ID | | |
| | | NO: 804) | | |
| | LNP | G025420 TRAC (SEQ ID NO: 464), Sp | 1 | ug/ml |

| | | |
|---|---|---|
| | Cas9 mRNA | |
| AAV | Homology directed repair construct | 3e5 GC/cell |
| | (SEQ ID NO: 106) encoding CD70 | |
| | CAR (SEQ ID NO: 4) | |

On day 4, cells were mixed, counted, and TCAM was added to adjust cell density to 0.5×10⁶ cells/mL prior to incubation for an additional 24 hrs.

On day 5, T cells were transferred and incubated in T cell expansion media (TCEM). From days 5-11 cells were expanded at 37° C. Cell counts were collected daily. After expansion, cells were harvested and counted with an NC200 Nucleocounter device (Chemometec) to determine cell viability and fold expansion.

For flow cytometric analysis, cells were washed the incubated in a cocktail of antibodies targeting CD4 (BioLegend 317434), CD8a (BioLegend 301046), CD3 (BioLegend 300430), HLA-A2 (for certain HLA-A2+donors) (BioLegend 343320), HLA-A3 (for certain HLA-A3+donors) (BD Biosciences 747776), HLA-B7 (Miltenyi Biotec 130-120-234), HLA-A9 (for certain HLA-A9+donors) (Miltenyi Biotec 130-099-539), HLA-DR/DP/DQ (BioLegend 361712) as a surrogate readout of CIITA function, HLA-Bw4 (for certain HLA-B alleles) (Miltenyi Biotec 130-103-851), CD45RA (BioLegend 304126), CD62L (BioLegend 304820), CD70-Fc (Sino Biological 10780-H01H) as a primary antibody then detected using labeled Anti-Fc (BioLegend 410708) to detect CAR insertion, and VioKrome Live/Dead dye (Beckman Coulter C36628) to assay viability. Flow cytometry data was acquired on a Cytoflex LX instrument (Beckman Coulter) and analyzed using the FlowJo software.

Table 16 and FIG. 10 show surface protein expression and cell viability as detected by flow cytometry. CD3 is a surrogate marker for TRAC editing. HLA-DR/DQ/DP-indicates successful disruption of CIITA. HLA-A- and HLA-B-status is reported using the antibody appropriate to the donor's genotype. Tscm (CD45RA+, CD62L+) and Tcm (CD45RA−, CD62L+) are indicative of memory cell populations. Table 17 and FIG. 10 show the C to T editing at TGFBR2 or CD70, reported as “TGFBR2—” or “CD70-” in FIG. 10. The percentage fully edited allo-CD70 CAR T cells was estimated as a mean of about 54% by multiplying the percentage of HLA-A-, HLA-B-, CD3—, CAR+ cells and % C to T editing at TGFBR2 and at CD70.

TABLE 16
Percent of engineered cells with the specified cell
surface phenotype as determined by flow cytometry.

| Assessment | Phenotype | Donor 1 | Donor 2 | Donor 3 | Mean | SD |
|---|---|---|---|---|---|---|
| TRAC KO | CD3− | 88.7 | 93.4 | 92.3 | 91.5 | 2.5 |
| or insertion | | | | | | |
| at TRAC | | | | | | |
| HLA-A KO | HLA-A− | 82.0 | 86.2 | 80.8 | 83.0 | 2.8 |
| HLA-B KO | HLA-B− | 96.9 | 97.4 | 89.7 | 94.7 | 4.3 |
| CIITA KO | HLA-DR/DP/DQ− | 91.6 | 92.8 | 86.5 | 90.3 | 3.3 |
| CD70 CAR | CAR+ | 88.7 | 91.5 | 84.0 | 88.1 | 3.8 |
| insertion | | | | | | |
| | Fully edited | 52.0 | 60.0 | 49.0 | 53.7 | 5.7 |
| | Viability (post-thaw) | 89.2 | 89.0 | 83.6 | 87.3 | 3.2 |
| Tscm + | CD62L+CD45RA+ | 93.6 | 90.5 | 86.2 | 90.1 | 3.7 |
| Tcm | & | | | | | |
| Memory | CD62L+CD45RA− | | | | | |
| cells | | | | | | |

TABLE 17
Percent C to T editing in engineered cells by NGS

| Locus | Donor 1 | Donor 2 | Donor 3 | Mean | SD |
|---|---|---|---|---|---|
| CD70 | 93.1 | 95.4 | 93.4 | 94.0 | 1.3 |
| TGFBR2 | 94.0 | 92 | 88.8 | 91.6 | 2.6 |

### Example 8: Expression of Activation Markers in Allo CD70 CAR T Cells Following Incubation with Renal Cancer Cells

CD70 CAR T cells were assessed for activation markers including CD69, CD107a, and CD25 upon co-culturing with 786-O renal cancer cells (RCC).

### 8.1. Co-Culture Assay

T cells were engineered and prepared as described in Example 7. The three engineered batches of cells from three donor sources were tested in triplicate as Lot A, Lot B, and Lot C.

On day 1, T cells were thawed and incubated in TCAM overnight at 37° C. 786-O RCC cells were counted and then centrifuged at 500×g for 5 minutes at room temperature. 786-O cells were resuspended in TCAM without cytokines, added to 96-well plates, and incubated to attach overnight at 37° C.

On day 2, rested CAR-T cells were harvested, counted, and resuspended in fresh TCAM. To set up co-cultures, CAR-T cells were added to wells of 786-O cells at 3:1, 1:1, or 1:3 E:T ratios. Recombinant Human TGFβ (R&D Systems, Cat. 7754-BH-100—CF) was added to each well at a concentration of 50 ng/mL.

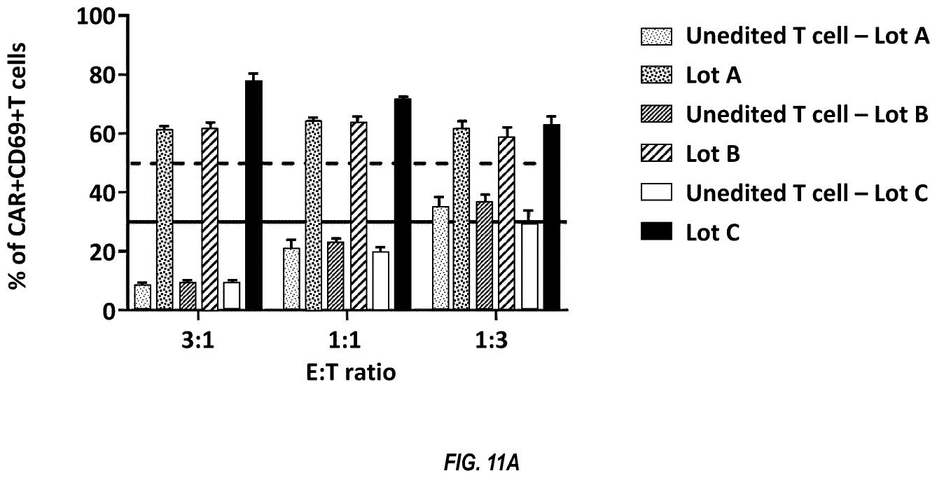

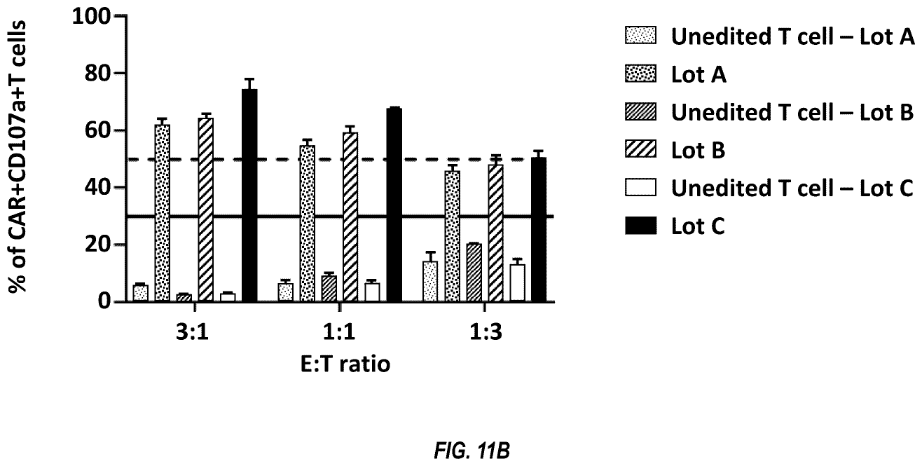

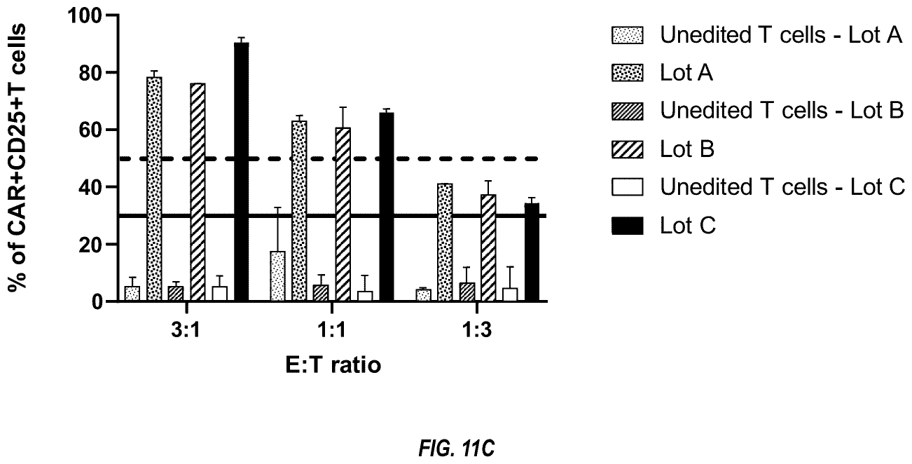

On day 3, following 24 hours of co-culture of CAR-T cells and tumor cells, the cells were removed from the incubator and washed to prepare for staining. For flow cytometric analysis, cells were washed and then incubated in a cocktail of antibodies targeting CD69 (BioLegend 310936), CD25 (BioLegend 356142), CD107a (BioLegend 328618), CD27 (BioLegend 356410), LAG3 (BioLegend 11C3C65), PD-1 (BioLegend 329952), anti-GS Linker (Genscript CA3152272), and Fixable Near IR Live/Dead dye (Invitrogen L34976) to assay viability. Flow cytometry data were acquired on a Cytoflex LX instrument (Beckman Coulter) and analyzed using the FlowJo software. Results in Tables 18, 19, and 20 and FIGS. 11A, 11B, and 11C show the increased cell surface expression of CD69, CD107a, and CD25, respectively, for CD70-CAR-T engineered cells with allogeneic (HLA-A KO, HLA-B KO, CIITA KO) and immune enhancing (CD70 KO, TGFBR2 KO) knockouts upon co-culture with 786-O tumor cells, relative to unedited control T cells.

TABLE 18
Percent of CD70 CAR T cells that are CD69+

| | 3:1 E:T Ratio | | | 1:1 E:T Ratio | | | 1:3 E:T Ratio | | |
|---|---|---|---|---|---|---|---|---|---|
| | Mean | SD | | Mean | SD | | Mean | SD | |
| Cell | % CD69+ | % CD69+ | | % CD69+ | % CD69+ | | % CD69+ | % CD69+ | |
| Conditions | Cells | Cells | N | Cells | Cells | N | Cells | Cells | N |
| Unedited Lot A | 8.9 | 0.8 | 3 | 21.3 | 2.9 | 3 | 35.0 | 3.5 | 3 |
| Lot A | 61.6 | 0.9 | 3 | 64.6 | 0.9 | 3 | 61.8 | 2.9 | 3 |
| Unedited Lot B | 9.6 | 0.2 | 3 | 23.3 | 1.4 | 3 | 37.0 | 2.4 | 3 |
| Lot B | 62.0 | 1.5 | 3 | 63.9 | 1.7 | 3 | 58.8 | 3.2 | 3 |
| Unedited Lot C | 9.6 | 0.5 | 3 | 19.9 | 1.8 | 3 | 30.4 | 3.3 | 3 |
| Lot C | 78.2 | 2.3 | 3 | 71.9 | 0.9 | 3 | 63.1 | 2.8 | 3 |

TABLE 19
Percent of CD70 CAR T cells that are CD107a+

| | 3:1 E:T Ratio | | | 1:1 E:T Ratio | | | 1:3 E:T Ratio | | |
|---|---|---|---|---|---|---|---|---|---|
| | Mean | SD | | Mean | SD | | Mean | SD | |
| Cell | % CD107a+ | % CD107a+ | | % CD107a+ | % CD107a+ | | % CD107a+ | % CD107a+ | |
| Conditions | Cells | Cells | N | Cells | Cells | N | Cells | Cells | N |
| Unedited Lot A | 5.8 | 0.3 | 3 | 6.3 | 1.4 | 3 | 14.3 | 3.2 | 3 |
| Lot A | 61.8 | 2.1 | 3 | 54.7 | 2.1 | 3 | 45.6 | 2.2 | 3 |
| Unedited Lot B | 2.7 | 0.1 | 3 | 9.0 | 0.9 | 3 | 20.0 | 0.4 | 3 |
| Lot B | 64.3 | 1.4 | 3 | 59.4 | 2.1 | 3 | 47.9 | 3.3 | 3 |
| Unedited Lot C | 2.8 | 0.4 | 3 | 6.4 | 1.3 | 3 | 12.9 | 2.3 | 3 |
| Lot C | 74.4 | 3.5 | 3 | 67.4 | 0.5 | 3 | 49.8 | 2.7 | 3 |

TABLE 20
Percent of CD70 CAR T cells that are CD25+

| | 3:1 E:T Ratio | | | 1:1 E:T Ratio | | | 1:3 E:T Ratio | | |
|---|---|---|---|---|---|---|---|---|---|
| | Mean | SD | | Mean | SD | | Mean | SD | |
| Cell | % CD25+ | % CD25+ | | % CD25+ | % CD25+ | | % CD25+ | % CD25+ | |
| Conditions | Cells | Cells | N | Cells | Cells | N | Cells | Cells | N |
| Unedited Lot A | 5.2 | 3.2 | 3 | 17.2 | 15.7 | 3 | 4.2 | 0.6 | 3 |
| Lot A | 78.1 | 2.5 | 3 | 62.9 | 2.0 | 3 | 41.0 | 0.1 | 3 |
| Unedited Lot B | 5.1 | 1.7 | 3 | 5.9 | 3.4 | 3 | 6.3 | 5.6 | 3 |
| Lot B | 76.0 | 0.1 | 3 | 60.8 | 7.1 | 3 | 37.4 | 4.8 | 3 |
| Unedited Lot C | 5.0 | 3.9 | 3 | 3.3 | 5.8 | 3 | 4.4 | 7.7 | 3 |
| Lot C | 90.4 | 1.8 | 3 | 66.1 | 1.2 | 3 | 34.4 | 1.9 | 3 |

### Example 9: Re-Challenging Allo-CD70 CAR-T Cells with 786-O or ACHN Tumor Cells

### 9.1 Thawing and Resting CAR-T and Control T-Cells

T-cells were engineered as described in Example 7.

Three different lots (A, B, or C) of CD70 allogenic anti-CD70 CAR-T cells and paired unedited negative control cells were assessed for potency in a serial re-challenge assay against either 786-O cells or ACHN renal tumor cells in the presence of TGFB1.

On Day 0, cryopreserved anti-CD70 CAR-T cells of the three lots and unedited control T cells were thawed, washed, and suspended in TCAM at a concentration of 1.0×10⁶ cells/mL and rested overnight in a 37° C. incubator.

9.2 Assay Setup with Adherent Target Cells

On Day 0, the 786-O GFP-Luciferase tumor cells and ACHN GFP-Luciferase tumor cells were harvested and counted. 200,000 786-O and 300,000 ACHN tumor cells were plated per well in a sterile 24-well clear TC-treated flat-bottom plate (Corning, Cat. 354408) and allowed to adhere by overnight incubation at 37° C.

### 9.3 Re-Challenge

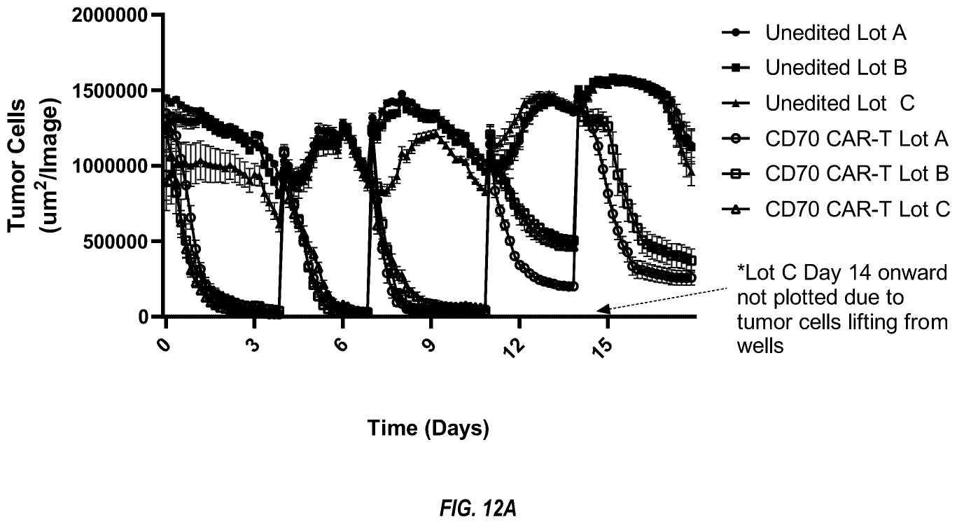

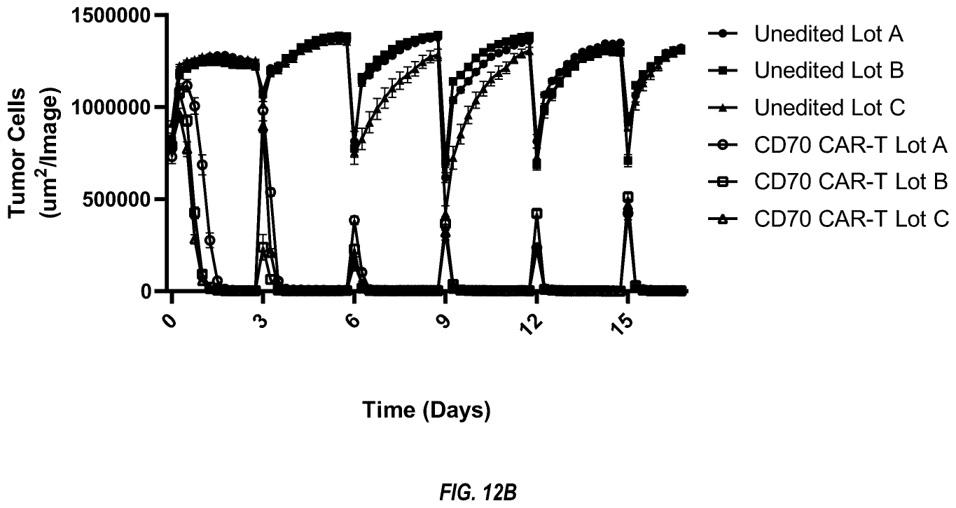

Engineered anti-CD70 CAR-T cells and unedited control T cells were challenged with 786-O or ACHN cells on Day 1, Day 4, and every three days thereafter. On day 1, 1 mL of media was removed from the tumor cell cultures and replaced with T cells with normalized CAR % for a final E: T ratio of 1:3. For subsequent re-challenges, one day before the re-challenge, cancer cells were pre-seeded as described in Example 9.2. On days of re-challenge, the co-culture 24-well plates from the prior cycle were removed from the Incucyte instrument and centrifuged at 500 RCF for five minutes. After centrifugation, 1 mL of the supernatant was aspirated, and the remaining cells were mixed. 500 μL of cell suspension from the prior cycle was added to the newly pre-seeded 786-O or ACHN plates for the re-challenge. The 24-well flat-bottom plates were transferred to the Incucyte instrument to continue monitoring the cytotoxicity. Throughout the assay recombinant Human TGFβ (R&D Systems, Cat. 7754-BH-100—CF) was added to each well at a concentration of 50 ng/ml, for each cycle of re-challenge. Results for the re-challenge are shown in FIG. 12A for the 786-O cell line and FIG. 12B for the ACHN cell line.

### Example 10—In Vivo Assessment of Allogeneic Anti-CD70 CAR Against Large RCC Tumors

The potency of engineered CD70 CAR-T cells was assessed in duplicate in a CD70 antigenhigh 786-O Renal Cell Carcinoma (RCC) tumor model. Thirty-nine female NOG mice were engrafted with 10e6 (1×10⁷) 786-O-GFP tumor cells followed by the injection of either Lot A or Lot C anti-CD70 CAR-T cells. Engineered T-cells were injected at three different doses (10e6, 3e6, and 1e6 (1×10⁷, 3×10⁶, and 1×10⁶, respectively)) when the solid tumors reached an approximate average volume of 450 mm3.

### 10.1 Engineering Anti-CD70 CAR-T Cells

T cells were engineered as described in Example 7.

### 10.2 Assessing Tumor Regression in 786-O-GFP Model

For the in vivo efficacy study, 786-O-GFP-Luc2 cells were harvested from culture, washed twice with HBSS (Gibco, Cat. No. 14025-092) and resuspended at 10e6 cells in 400 μL of HBSS for injections. To assess the efficacy of CD70 CAR-T cell Lots A and C, 39 female NOG mice (Taconic) were dosed subcutaneously with 786-O tumor cells on the right flank. The animals were monitored twice a week for tumor growth by caliper measurements and their tumor volumes were recorded. Once the tumors reached an average of ~450 mm³ in volume, animals were randomized on day 34 post engraftment followed by T cell infusion. Different lots of anti-CD70 CAR T cells engineered as described above were thawed, washed with HBSS (Gibco, Cat. No. 14025-092) and resuspended at the respective doses of 10e6, 3e6 and 1e6 in 150 μL of HBSS for injections. 6 mice per T-cell group were dosed by tail vein injections in the tumor engrafted animals.

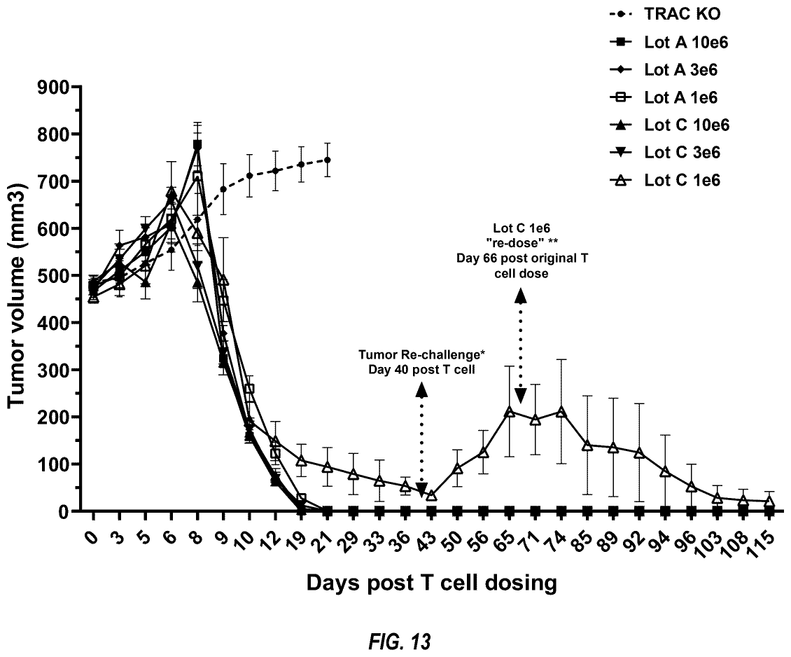

Tumor caliper measurements were done twice a week post T cell dosing along with recording body weights post T-cell dosing on days 3, 5, 6, 8, 10, 12, 15, 19, 21, 29, 33, 36, 43, 50, 56, 65, 71, 74, 85, 89, 92, 96, 99, 103, 108, and 115. The mice were prepared to be measured by restraining the animal securely and shaving the excess fur off the right-side flank of the animal. The shaved area was then wiped down with an alcohol swab to clearly visualize the tumor and the length and the width of the tumor was measured using calipers. Tumor volumes were calculated as (((Length+Width)/2/2)\hat{ }3)*3.14*1.33. FIG. 13 shows the average tumor volume data for each group dosed with different lots of engineered CAR-T cells from day of randomization (Day 34 post engraftment) until study termination. Engineered anti-CD70 CAR-T cells were dosed on day 34 post tumor engraftment. The animals were monitored for over two weeks post tumor clearance to ensure there are no remaining tumor cells at the engraftment site post complete regression. On day 40 post T cell dosing, the animals that were dosed with 10e6 and 3e6 of Lot A and Lot C anti-CD70 CAR T cells were re-challenged with 10e6 of 786-O-GFP-Luc2 cells again on their right flank to assess the potency and persistence of the engineered anti-CD70 CAR-T cells in vivo. The animals were monitored to record any tumor outgrowth until the end point of the study.

### Example 11—In Vivo Study of Anti-CD70 Allo CAR-T Cells in Renal Cell Carcinoma Patient Derived Xenograft (RCC PDX) Model

The efficacy of anti-CD70 allo CAR-T cells was assessed in a renal cell carcinoma PDX model against 11 patient-derived tumors that express CD70. Female PDX mice were engrafted with a 2x2 mm patient-derived tumor fragment, followed by the injection of anti-CD70 CAR-T cells. CAR-T cells were injected at a dose of 5e6 cells when the solid tumors reached an approximate average volume of 400-600 mm3.

### 11.1 Engineering Anti-CD70 CAR-T Cells

T cells were engineered as described in Example 7.

### 11.2 Anti-CD70 Allo CAR-T Cells Induced Tumor Regression in 9 Out of 11 RCC PDX Models

### Tumor Fragment Revival and Implantation

For revival of the solid tumor fragments, frozen tumor fragments were thawed in a 37° C. water bath. Ten female NSG mice were inoculated with the patient-derived tumor fragments on a single flank for passaging before being implanted in PDX mice. Each mouse received an injection of buprenorphine 30 minutes before tumor fragment implantation. Once the tumor volume reached approximately 700 to 1000 mm3, tumors were harvested and processed for tumor chunk inoculation in PDX mice.

After harvesting, the tumors were washed with PBS and were sliced into 2-3 mm² tumor fragments. PDX mice received an injection of buprenorphine 30 minutes before tumor fragment implantation and were inoculated with a 2×2 mm tumor fragment.

Tumor volume was calculated using the following equation: (longest diameter * shortest diameter²)/2. Once tumors were of appropriate size to begin the study, tumors and body weights were measured at least 2 times per week for the duration of the study. When the average tumor volume reached 400-600 mm³, three mice were chosen and dosed with T cells within 24 hours of randomization.

### Anti-CD70 Allo CAR T Cell Dosing

Anti-CD70 allo CAR-T cells were counted, and viability was recorded on the day of CAR-T cell injection. Cells were thawed at 37° C. The cells were combined with 35 mL 10% FBS/RPMI warmed to 37° C. The cells were resuspended in 10 mL of HBSS and centrifuged at 500×g for 5 minutes at 37° C.

Following centrifugation, the volume of the cells was adjusted to 5 million CAR T cells per 200 μL of HBSS. Each group of mice was injected within 1 hour of thawing the CAR-T cells. CAR-T cells were dosed by tail vein injection in the tumor engrafted animals. Different models were dosed at different days based on the growth kinetics of the model and the time it takes for three mice to reach the dosing volume range of 400 mm³ to 600 mm³. CAR-T cells were injected when the tumor volumes reached 400-600 mm³. The study for each model was terminated four weeks after dosing CAR-T cells, unless otherwise stated. Day 1 was defined as day of CAR-T cell dosing.

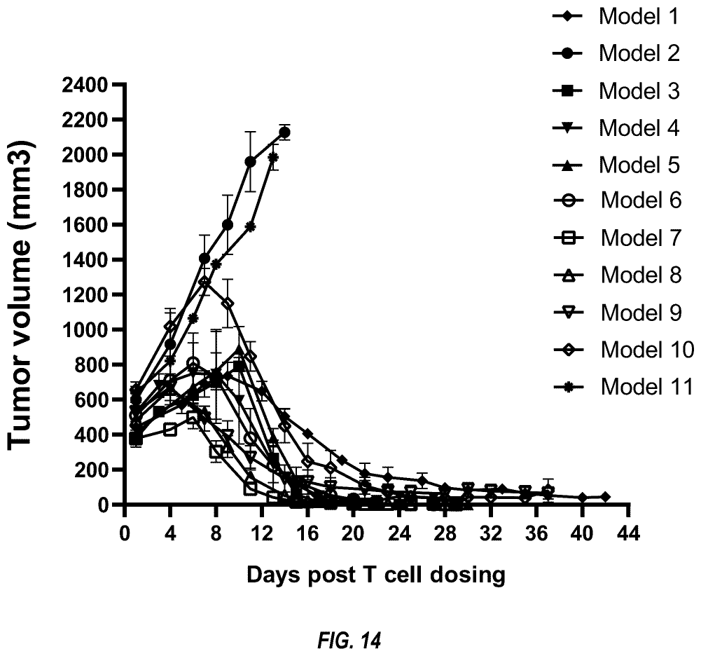

Table 21 and FIG. 14 show the tumor volume data for each group dosed with allo CD70 CAR-T cells from day of dosing until study termination.

TABLE 21
Tumor volumes from all groups post T-cell dosing.

| | Days post | | | |
|---|---|---|---|---|
| | CAR-T cell | Tumor volume (mm³) | | |
| Model | dosing | Mouse 1 | Mouse 2 | Mouse 3 |
| 1 | 1 | 498.29 | 425.96 | 407.62 |
| | 5 | 667.27 | 506.75 | 536.32 |
| | 7 | 838.82 | 631.03 | 599.27 |
| | 9 | 880.83 | 715.06 | 610.82 |
| | 12 | 760.3 | 601.66 | 584.26 |
| | 14 | 592.09 | 465.46 | 457.22 |
| | 16 | 412.55 | 397.71 | 408.74 |
| | 19 | 245.87 | 204.45 | 312.5 |
| | 21 | 105.73 | 134.19 | 293.75 |
| | 23 | 79.1 | 119.14 | 270.15 |
| | 26 | 81.83 | 100.44 | 225.82 |
| | 28 | 69.23 | 76.83 | 144.18 |
| | 30 | 64.52 | 73.17 | 113.26 |
| | 33 | 68.94 | 79.15 | 120.14 |
| | 35 | 62.25 | 54.68 | 79.59 |
| | 37 | 47.68 | 45.75 | 64.85 |
| | 40 | 32 | 32 | 59.88 |
| | 42 | 45.16 | 27.22 | 60.34 |
| 2 | 1 | 445.24 | 682.58 | 668.66 |
| | 4 | 659.09 | 826.71 | 1262.84 |
| | 7 | 1141.49 | 1503.18 | 1575.57 |
| | 9 | 1261.71 | 1790.04 | 1745.74 |
| | 11 | 1732.21 | 2293.7 | 1852.59 |
| | 14 | 2083.76 | Mouse | 2171.84 |
| | | | Euthanized | |
| 3 | 1 | 376.99 | 461.7 | 296.42 |
| | 3 | 548.54 | 546.51 | 501.19 |
| | 6 | 664.84 | 631.34 | 580.66 |
| | 8 | 715.1 | 736.48 | 647.5 |
| | 10 | 756.41 | 835.33 | 780.41 |
| | 13 | 277.61 | 293.13 | 219.82 |
| | 15 | 32 | 112.43 | 32 |
| | 17 | 4 | 32 | 4 |
| | 20 | 0 | 13.5 | 0 |
| | 22 | 0 | 13.5 | 0 |
| | 24 | 0 | 0 | 0 |
| | 27 | 0 | 0 | 0 |
| | 29 | 0 | 0 | 0 |
| 4 | 1 | 378.93 | 552.38 | 649.12 |
| | 3 | 554.35 | 705.58 | 781.14 |
| | 6 | 401.76 | 937.26 | 910.8 |
| | 8 | 240.05 | 1069.96 | 924.18 |
| | 10 | 125.28 | 965.23 | 691.48 |
| | 13 | 32 | 392.9 | 267.66 |
| | 15 | 13.5 | 147.75 | 178.99 |
| | 17 | 4 | 63.48 | 81.67 |
| | 20 | 0.5 | 32 | 55.63 |
| | 22 | 0.5 | 32 | 38.12 |
| | 24 | 4 | 55.32 | 81.98 |
| | 27 | 4 | 32 | 79.47 |
| | 29 | 4 | 13.5 | 32 |
| 5 | 1 | 382.94 | 346.73 | 496.5 |
| | 6 | 673.78 | 511.85 | 830.89 |
| | 8 | 727.3 | 598.35 | 960.65 |
| | 10 | 868.32 | 677.66 | 1122.69 |
| | 13 | 332.63 | 328.11 | 487.37 |
| | 15 | 147.41 | 132.81 | 139.9 |
| | 17 | 32 | 0 | 32 |
| | 20 | 32 | 0 | 32 |
| | 22 | 13.5 | 0 | 4 |
| | 24 | 13.5 | 0 | 4 |
| | 27 | 4 | 0.5 | 0.5 |
| | 30 | 0.5 | 0.5 | 0.5 |
| 6 | 1 | 322.04 | 445.75 | 762.55 |
| | 4 | 475.02 | 695.8 | 958.05 |
| | 6 | 510.76 | 808.56 | 1106.56 |
| | 8 | 418.34 | 512.47 | 1249.5 |
| | 11 | 213.13 | 201.84 | 721.39 |
| | 13 | 93.88 | 54.49 | 516.16 |
| | 15 | 80.2 | 13.5 | 294.77 |
| | 18 | 32 | 4 | 126.99 |
| | 20 | 13.5 | 4 | 72.31 |
| | 22 | 4 | 4 | 13.5 |
| | 25 | 4 | 0 | 0.5 |
| | 27 | 4 | 0 | 0.5 |
| | 29 | 4 | 0.5 | 0 |
| 7 | 1 | 423.55 | 376.98 | 333.39 |
| | 4 | 501.41 | 404.05 | 384.55 |
| | 6 | 622.48 | 473.94 | 401.76 |
| | 8 | 424.67 | 271.67 | 215.35 |
| | 11 | 154.15 | 32 | 84.12 |
| | 13 | 90.67 | 4 | 32 |
| | 15 | 32 | 4 | 13.5 |
| | 18 | 32 | 4 | 4 |
| | 20 | 13.5 | 0.5 | 4 |
| | 22 | 13.5 | 0 | 0.5 |
| | 25 | 13.5 | 0 | 0.5 |
| | 27 | 13.5 | 0 | 0.5 |
| | 29 | 4 | 0 | 0 |
| 8 | 1 | 459.54 | 497.58 | 484.85 |
| | 4 | 697.68 | 666.55 | 619.99 |
| | 7 | 595.32 | 515.26 | 454.33 |
| | 9 | 272.96 | 458.46 | 264.94 |
| | 11 | 115.4 | 213.09 | 143.36 |
| | 14 | 32 | 89.86 | 32 |
| | 16 | 13.5 | 32 | 13.5 |
| | 18 | 4 | 13.5 | 13.5 |
| | 21 | 4 | 4 | 4 |
| | 23 | 4 | 4 | 4 |
| | 25 | 0.5 | 4 | 4 |
| | 28 | 0 | 4 | 4 |
| | 29 | 0 | 4 | 4 |

| | Days post | | | | |
|---|---|---|---|---|---|
| | CAR-T cell | Tumor volume (mm³) | | | |
| Model | dosing | Mouse 1 | Mouse 2 | Mouse 3 | Mouse 4 |
| 9 | 1 | 387.47 | 607.09 | 625.6 | 499.31 |
| | 4 | 466.66 | 837.43 | 709.41 | 674.16 |
| | 7 | 315.44 | 663.68 | 510.45 | 482.23 |
| | 9 | 151.95 | 567.01 | 438.22 | 415.45 |
| | 11 | 82.55 | 416.85 | 304.07 | 271.35 |
| | 14 | 13.5 | | 281.74 | 154.75 |
| | 16 | 13.5 | | 254.05 | 120.47 |
| | 18 | 4 | | 190.01 | 108.63 |
| | 21 | 4 | | 179.89 | 78.51 |
| | 23 | 4 | | 151.5 | 69.32 |
| | 25 | 4 | | 146.32 | 60.54 |
| | 28 | 4 | | 135.63 | 50.05 |
| | 29 | 4 | | | |
| | 30 | | | 126.56 | 48.08 |
| | 32 | | | 116.52 | 38.84 |
| | 35 | | | 108.89 | 33.31 |
| | 37 | | | 99.24 | 32 |
| 10 | 1 | 680.75 | 775.55 | 593.69 | 575.55 |
| | 4 | 990.43 | 1250.36 | 1077.75 | 753.69 |
| | 7 | 1298.27 | 1434.88 | 1298.35 | 1060.67 |
| | 9 | 1066.99 | 1536.5 | 1121.55 | 876.52 |
| | 11 | 917.7 | 1046.1 | 752.15 | 673.89 |
| | 14 | | 631.07 | 303.27 | 414.85 |
| | 16 | | 444.41 | 88.21 | 208.93 |
| | 18 | | 407.03 | 85.78 | 142.83 |
| | 21 | | 196.74 | 32 | 97.91 |
| | 23 | | 124.27 | 32 | 62.95 |
| | 25 | | 92.28 | 13.5 | 32 |
| | 28 | | 79.96 | 0 | 32 |
| | 29 | | | 0 | |
| | 30 | | 74.49 | | 13.5 |
| | 32 | | 69.29 | | 13.5 |
| | 35 | | 65.07 | | 13.5 |
| | 37 | | 149.02 | | 13.5 |
| 11 | 1 | 626.94 | 609.49 | 691.77 | 621.19 |
| | 4 | 750.93 | 836.48 | 846.82 | 851.01 |
| | 6 | 1055.96 | 1078.45 | 1085.02 | 1038.77 |
| | 8 | 1364.76 | 1387.06 | 1390.72 | 1352.93 |
| | 11 | 1553.7 | 1576.94 | 1550.14 | 1672.82 |
| | 13 | 1842.6 | 2027.76 | | 2084.18 |

Example 12: G Banded Karyotyping of Engineered Allo-CD70-CAR-T Cells

Karyotyping of 200 metaphase spreads was performed in lots of engineered allo-CD70-CAR-T cells to evaluate for potential gross chromosomal abnormalities. In these studies, the allo-CD70 CAR-T cells are engineered with site-specific anti-CD70 CAR transgene insertion into the TRAC locus using a Streptococcus pyogenes CRISPR/Cas9 (SpyCas9) cleavase editor, in combination with gene knockout of HLA-A, HLA-B, CIITA, TGFBR², and CD70 using a Neisseria meningitidis Cas9 (Nme2Cas9) cytosine base editor (Group 1). The ex vivo delivery of these editing components is accomplished by transfection of LNPs encapsulating sgRNAs and/or mRNA. To test the potential genotoxicity impacts of the editing and delivery modalities, these lots were compared to donor-matched controls that were not edited (Group 2, control), or edited by electroporation of SpyCas9 cleavase RNPs targeting the same 6 genes (Group 3).

### 12.1. T Cell Engineering

Engineered allo-CD70 CAR-T cells from Example 7 were tested alongside donor-matched CAR-T cells that were engineered by editing the same targets but using electroporation of Cas9 cleavase RNPs. For this, T cells were thawed and rested overnight in T cell media (CTS Optimizer (Thermofisher) supplemented with 5% human serum, IL-2 (200 U/mL), IL-7 (5 ng/ml), IL-15 (5 ng/ml)) and activated using TransAct (Miltenyi). After 48 hours of activation, the cells were harvested, counted, and resuspended in nucleofection buffer (P3 Primary Cell Nucleofector solution+Supplement 1; Lonza-V4SP-3096) at 1e6 cells/20 μL. RNP complexes were prepared for each by mixing SpyCas9 protein with sgRNA at 2:1 guide: cas9 ratio and incubating for 10 minutes at room temperature. RNPs for all targets were mixed with the cell suspension at a 0.8 μM concentration. The cell-RNP mix was transferred to a Lonza 96-well Nucleocuvette Plate and electroporated using EH-115 program on the Lonza 4D-Nucleofector 96-well Unit. Post electroporation, the cells were transferred to a 24-well plate containing CD70 CAR encoding AAV. Edited cells were later moved to a GRex culture plate (Wilson Wolf Manufacturing) for expansion for 5-7 days. Editing and CAR insertion were confirmed via flow and/or amplicon-sequencing.

### 12.2. Metaphase Preparation and Analysis

Engineered CAR T cells were thawed or taken from culture and cultured in cytokine containing media for 72 hours to obtain optimal logarithmic growth phase. After 72 hours, cells were counted and adjusted to 1e6 cells/mL and incubated with Colcemid (0.1 μg/mL) for 2 hours. Post incubation, the cells were centrifuged and suspended in 75 mM KCl hypotonic solution for a 30 minutes incubation at 37° C. Post incubation, cells were washed and suspended in a 3:1 methanol to acetic acid fixative. Cells were fixed at room temperature for 20 minutes for G banded karyotyping. Fixed cells were prepared onto slides for each sample for staining and imaging. For each editing condition 200 metaphase spreads were analyzed and compared in a pairwise manner to the donor matched unedited control T cells prepared similarly (across N=3 paired donors). Statistical analysis was done by Fishers Exact test comparing the paired edited vs. non-edited cells for each indicated chromosomal abnormality.

### 12.3. Karyotyping

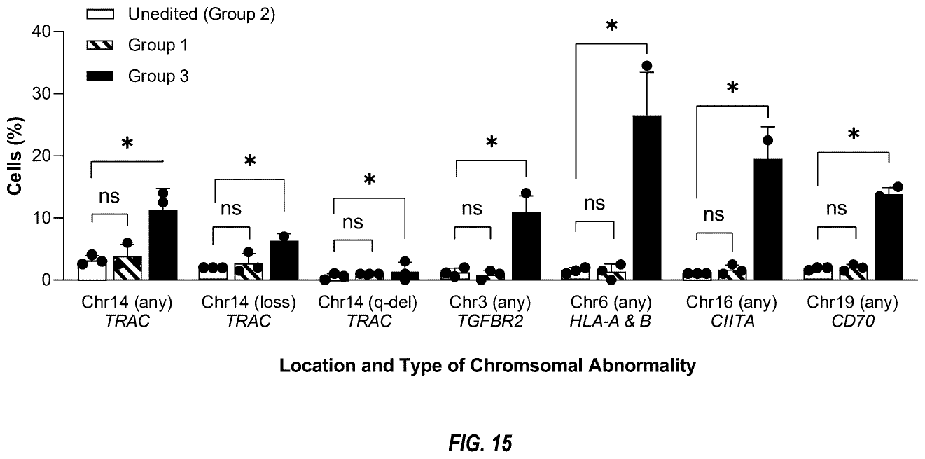

Karyotyping of allo-CD70 CAR-T lots engineered using cleavase based CD70-CAR insertion and cytosine base-editor knockout of HLA-A, HLA-B, CIITA, TGFBR2, and CD70 did not show statistically significant increases in gross chromosomal abnormalities relative to unedited controls in any targeted chromosome (FIG. 15, Table 22). In contrast, T cells edited with electroporation of Cas9 RNPs showed a striking loss of chromosomal integrity, with each edited chromosome having abnormalities in >10% of cells. The predominant aberration was complete chromosome loss, but other common events included p/q-arm deletions, truncations, and translocations, all near the targeted regions.

TABLE 22
G banded karyotyping

| | Unedited (Group 2) | | | Group 1 | | | Group 3 | | |
|---|---|---|---|---|---|---|---|---|---|
| | Mean | SD | N | Mean | SD | N | Mean | SD | N |
| Chr14 (any) | 6.3 | 1.5 | 3 | 7.7 | 3.8 | 3.0 | 22.7 | 6.8 | 3 |
| TRAC | | | | | | | | | |
| Chr14 (loss) | 4.0 | 0.0 | 3 | 5.3 | 3.2 | 3.0 | 12.7 | 2.3 | 3 |
| TRAC | | | | | | | | | |
| Chr14 (q-del) | 1.0 | 1.0 | 3 | 2.0 | 0.0 | 3.0 | 2.7 | 3.1 | 3 |
| TRAC | | | | | | | | | |
| Chr3 (any) | 2.3 | 1.5 | 3 | 1.7 | 1.5 | 3.0 | 22.0 | 5.2 | 3 |
| TGFBR2 | | | | | | | | | |
| Chr6 (any) | 3.0 | 1.0 | 3 | 2.7 | 2.5 | 3.0 | 53.0 | 13.9 | 3 |
| HLA-A & B | | | | | | | | | |
| Chr16 (any) | 2.0 | 0.0 | 3 | 3.3 | 1.5 | 3.0 | 39.0 | 10.4 | 3 |
| CIITA | | | | | | | | | |
| Chr19 (any) | 3.7 | 0.6 | 3 | 4.0 | 1.0 | 3.0 | 27.7 | 2.1 | 3 |
| CD70 | | | | | | | | | |

### Example 13: Allogeneic Functional Analysis of Engineered CD70 CAR T Cells

### 13.1. In Vitro NK Cell Killing Assay

### Thawing & Resting Host NKs and Engineered Donor T Cells

Cryopreserved host HLA-I mismatched or HLA-C matched NK cells along with engineered donor T cells as described in Tables 23 and 24 were thawed and spun down at 500 xg for 5 minutes. T cells were then resuspended at a cell concentration of 1-2e6 cells/mL in T cell growth media (TCGM) composed of OpTmizer TCGM (Gibco, A1048501), Human Serum AB, HEPES 1M, GlutaMAX Supplement, and Penicillin-Streptomycin and further supplemented with recombinant human interleukin-2, 5 ng/mL IL-7, and 5 ng/mL IL-15. NK cells were resuspended at a cell concentration of 1-2e6 cells/mL in TCGM media with IL-2 and IL-15. Donor T cells and host NK cells were transferred to a 37° C. incubator and rested overnight.

### NK Killing Assay Setup

The following day engineered donor T cells were spun down and resuspended at 0.2e6/mL in TCGM with IL-2. 100 μL of donor T cells were plated in a 96 well U-Bottom plate in duplicates to a total T cell density of 20,000 per well. Host NK cells were resuspended at 1e6/mL in Phosphate Buffer Saline (PBS) (Corning, Cat. No. 21-040-CV) for Cell Trace Violet (CTV) staining. NK cells were stained with 0.5 μM of CTV and incubated for 15 minutes in a 37° C. incubator. Post incubation the cells were washed with TCGM media and spun down at 500 xg for 5 minutes. CTV labelled host NK cells were resuspended in pre-warmed TCGM media composed of OpTimizer TCGM at 4e6/mL. 50 μL of NK cells were added to the respective wells on top of plated donor T cells in duplicates so that the total NK cells are 200,000 per well, making the NK: T ratio of 10:1. Plates were transferred to a 37° C. incubator for 18-20 hours.

The following day, NK cell killing activity was evaluated by performing flow cytometry. 150 μL of cells were plated in 15 μL of DRAQ7 (Biolegend, Cat.No.-424001) viability dye to a final concentration of 1:200 in Phosphate Buffer Saline. The plate was read on either a Cytoflex LX or MACS Quant flow cytometry machine and data were collected from at least 100 μL to measure viability of T cells.

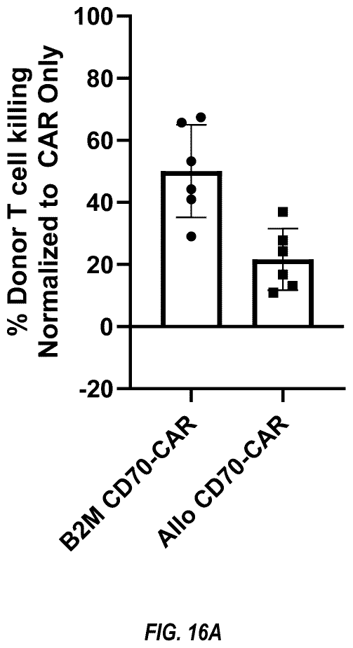

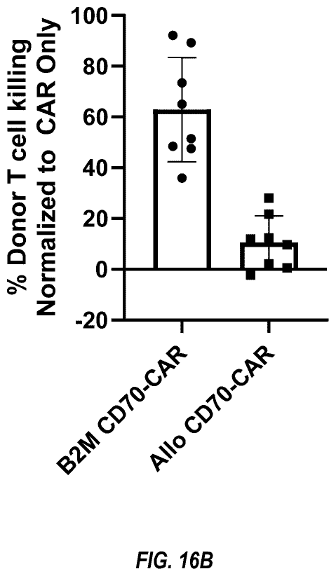

The percentage of T cell lysis was calculated by gating on CTV DRAQ7-negative cells and subtracting by 100. B2M CAR and allo CD70 CAR groups were normalized with CAR only groups. The average T cell killing for all engineered donor T cells was calculated and reported for mismatched host NK cells or HLA-C matched host NK cells in Tables 26 and 27, respectively, and in FIGS. 16A and 16B, respectively. HLA Class I genotype information for the host NK cells is shown in Table 23 and for donor T cells in Table 24. Edits for each respective donor and group are shown in Table 25.

TABLE 23
HLA Class I Genotype of Host NK Cells

| | | | | | |
|---|---|---|---|---|---|
| HLA-A | HLA-A | HLA-B | HLA-B | HLA-C | HLA-C |

Mismatched NKs

| | | | | | | |
|---|---|---|---|---|---|---|
| Host NK1MM | 23:17 | 30:02 | 57:03 | 58:02 | 06:02 | 07:18 |
| Host NK2MM | 02:01 | 23:17 | 45:01 | 81:01 | 16:01 | 18:01 |

HLA-C Matched NKs

| | | | | | | |
|---|---|---|---|---|---|---|
| HostNK1Matched | 24:02 | 74:01 | 35:12 | 53:01 | 04:01 | 06:02 |
| HostNK2Matched | 11:01 | 68:02 | 51:01 | 53:01 | 04:01 | 15:02 |
| HostNK3Matched | 02:01 | 29:02 | 07:02 | 14:02 | 07:02 | 08:02 |
| HostNK4Matched | 03:01 | 32:01 | 07:02 | 35:03 | 04:01 | 07:02 |
| HostNK5Matched | 02:01 | 24:02 | 39:02 | 51:01 | 07:01 | 07:02 |
| HostNK6Matched | 01:01 | 11:01 | 52:01 | 58:01 | 07:01 | 12:02 |
| HostNK7Matched | 01:01 | 01:01 | 07:02 | 08:01 | 07:01 | 07:02 |
| HostNK8Matched | 01:01 | 01:01 | 08:01 | 35:01 | 04:01 | 07:01 |

TABLE 24
HLA Class I Genotype of Engineered Donor T-Cells

| | HLA-A | HLA-A | HLA-B | HLA-B | HLA-C | HLA-C |
|---|---|---|---|---|---|---|
| DonorT1 | 02:01 | 23:01 | 15:01 | 44:03 | 04:01 | 04:01 |
| DonorT2 | 03:02 | 03:02 | 08:01 | 08:01 | 07:01 | 07:01 |
| DonorT3 | 02:01 | 03:01 | 07:02 | 07:02 | 07:02 | 07:02 |

TABLE 25
Edits Included in Each Donor Group

| Donor | Group Name | Edits |
|---|---|---|
| DonorT3 | CAR Only | CD70/TRAC KO + CD70 CAR |
| DonorT3 | B2M CAR | TGFBR2/TRAC/CIITA/CD70/B2M KO + CD70 |
| | | CAR |
| DonorT3 | Allo CD70 | TGFBR2/TRAC/CIITA/CD70/HLA-A/HLA-B |
| | CAR | KO + CD70 CAR |
| DonorT1 | CAR Only | CD70/TRAC KO + CD70 CAR |
| DonorT1 | B2M CAR | TGFBR2/TRAC/CIITA/CD70/B2M KO + CD70 |
| | | CAR |
| DonorT1 | Allo CD70 | TGFBR2/TRAC/CIITA/CD70/HLA-A/HLA-B |
| | CAR | KO + CD70 CAR |
| DonorT2 | CAR Only | CD70/TRAC KO + CD70 CAR |
| DonorT2 | B2M CAR | TGFBR2/TRAC/CIITA/CD70/B2M KO + CD70 |
| | | CAR |
| DonorT2 | Allo CD70 | TGFBR2/TRAC/CIITA/CD70/HLA-A/HLA-B |
| | CAR | KO + CD70 CAR |

TABLE 26
Average T cell killing (of all donors in group
indicated) by genotypically mismatched NK host

| | | | N (donor/ |
|---|---|---|---|
| Group | Average | SD | host combination) |
| B2M CAR | 50.12 | 14.93 | 6 (3 donors tested with |
| | | | 2 mismatched host NK) |
| Allo CD70 | 21.64 | 9.90 | 6 (3 donors tested with |
| CAR | | | 2 mismatched host NK) |

TABLE 27
Average T cell killing (of all donors in group indicated)
by genotypically HLA-C Matched NK host

| | | | N (donor/ |
|---|---|---|---|
| Group | Average | SD | host combination) |
| B2M CAR | 62.90 | 20.58 | 8 (DonorT1 vs HostNKMatched1/2; |
| | | | DonorT3 vs |
| | | | HostNKMatched3/4/5; DonorT2 vs |
| | | | HostNKMatched6/7/8 - |
| | | | of Tables 23 & 24) |
| Allo CD70 | 10.53 | 10.51 | 8 (same |
| CAR | | | combinations as above) |

### 13.2. MLR Assay/Host Vs Graft Assay

### Thawing & Resting Host PBMCs and Engineered Donor T Cells

Cryopreserved host PBMCs and unedited donor T cells as described in Tables 31A-31D were thawed at a cell concentration of 1-2e6 cells/mL into T cell growth media (TCGM) composed of OpTmizer TCGM (Gibco, A1048501), Human Serum AB (GeminiBio, 100-512), HEPES 1M (Gibco, 15630-080), GlutaMAX Supplement (Gibco, 35050-061), and Penicillin-Streptomycin (Gibco, 15070-063) and further supplemented with recombinant human interleukin-2 (Peprotech, Cat. 200-02), IL-7 (Peprotech, Cat. 200-07), IL-15 (Peprotech, Cat. 200-15) for T cells and only IL-2 for host PBMCs (Peprotech, Cat. 200-02). Cells were rested in 37° C. incubator overnight.

Assay Setup with Engineered Donor T Cells and Host PBMCs
Priming of Host PBMCs with Donor T Cells

The following day, unedited donor T cells were irradiated at 5000 rad (Program C) and spun down, and each group was resuspended at 1e6/mL in TCGM containing IL-2. Host PBMCs underwent CD56 depletion using CD56 microbeads from Miltenyi as described in manufacturer's protocol. The CD56 depleted PBMCs were then counted using Nexcelom Celleca cell counter and were collected and spun down followed by resuspending at 1e6 cells/mL in TCGM media with IL-2. In a 6-well G-Rex, the host PBMCs were added at 5-10e6 per well and an equal number of irradiated unedited donor T cells were added so that the E: T is 1:1 for priming. The well volume was brought to 30 mL with TCGM media containing recombinant human IL-2 and incubated at 37° C. for 7 days.

Killing Assay Setup with Primed Host PBMCs Vs Engineered Allo CD70 CAR T and Control T Cells

A vial of engineered allo CD70 CAR T cells and a vial of CAR Alone cells (as described in Table 32) were thawed six days after the priming step. Donor T cells were resuspended at a concentration of 1-2e6 cells/mL into T cell growth media (TCGM) and further supplemented with recombinant human interleukin-2 (Peprotech, Cat. 200-02), IL-7 (Peprotech, Cat. 200-07), and IL-15 (Peprotech, Cat. 200-15). The cells were rested at 37° C. overnight.

The following day the T cells were removed from the incubator and spun down. The T cells were counted using a Cellaca cell counter and resuspended at 1e6 cells/mL in PBS and then transferred to a 15 ml conical tube to stain with Cell Trace Far Red (CTFR) (Invitrogen, Cat No. C34564). The engineered Donor T cells were resuspended in PBS and stained with final concentration of 0.5 μM of CTFR. The cells were incubated for 15 minutes at 37° C. and then spun down. The cells were resuspended at 0.2e6 cells/mL in TCGM media without cytokines and plated with 100 μL of cells per well to a 96-well U-Bottom plate. One plate was prepared for each timepoint.

The primed host PBMCs were removed 7 days post incubation and collected. The cells were counted using Cellaca cell counter and resuspended at 1e6 cells/mL in PBS and transferred to a 15 mL conical tube. A total of 5-10 million host PBMCs were stained with Cell Trace Violet (Thermo Fisher, Cat. No. C34571) at 1 μM final concentration and incubated for 15 minutes at 37° C. The labelled host cells were then spun down at 500 xg for 5 minutes. CTV labelled host PBMCs were then resuspended at 1.2e6 cells/mL in pre-warmed TCGM media without cytokines. 50 μL of labelled host PBMCs were added to their designated wells on top of labeled engineered donor T cells to a final cell count of 60,000/well of PBMC to 20,000/well of Donor T cell (E: T=3:1). One plate was transferred on ice for the Day 0 timepoint. The remaining 3 plates were transferred to 37° C. for collection on Day 2, Day 3 or Day 4.

### Alloreactivity Readout by Flow Cytometry

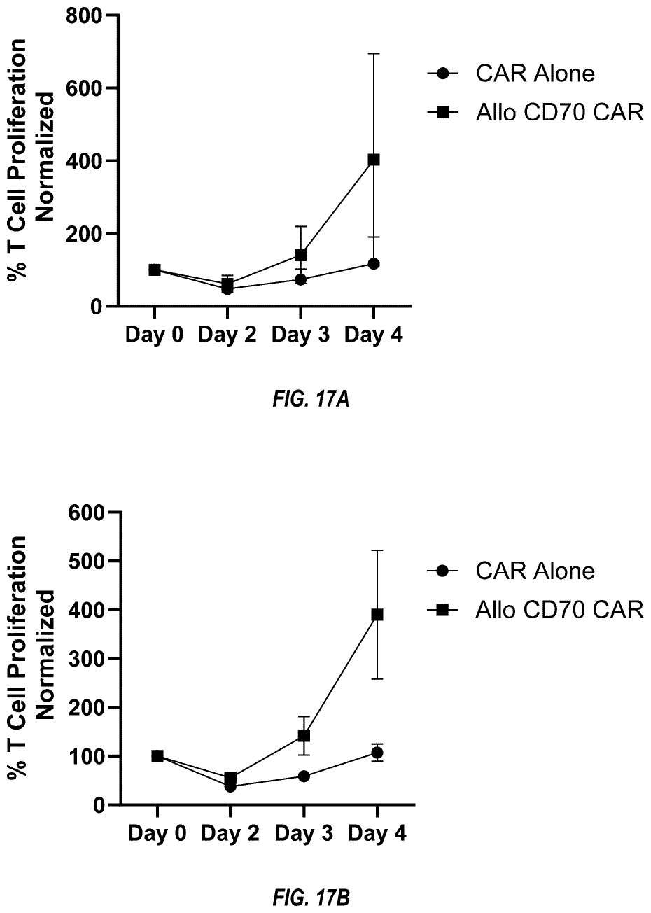

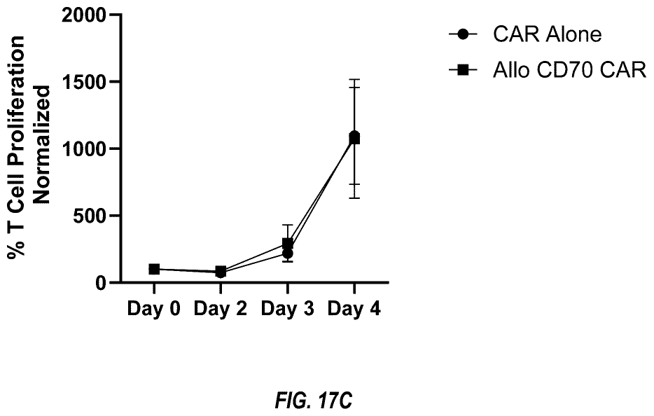

The flow cytometry readout was collected at Day 0, Day 2, Day 3, and Day 4. For the Day 0 timepoint, immediately following plate set up, 15 μL of viability dye, Sytox Green Nucleic Acid Stain (Invitrogen, Cat.No.-S7020) was added and brought to a final concentration of 1:2000 in PBS. The cells were mixed and incubated for 10 minutes at room temperature with protection from light. The cells were read on a MACS Quant flow cytometry machine to record the cell count data based on gating out dead cells (Sytox Green negative) and gating on CTFR positive cells for engineered donor T cells and CTV positive cells for host PBMCs. The same process was repeated to collect cell counts on Day 2, Day 3, and Day 4. Engineered donor T cell proliferation was recorded for all donor/host combinations examined and averaged, as shown in FIG. 17A and Table 28 (PBMC mismatched) and FIG. 17B and Table 29 (PBMC C-matched). FIG. 17C and Table 30 show engineered donor T cell proliferation in the presence of autologous PBMC. Cell counts were normalized for their respective group by calculating the Day 0 value to be 100%.

TABLE 28
Percent of T cell proliferation with
genotypically mismatched donors

| | | | | N (donor/ |
|---|---|---|---|---|
| Group | Day | Average | SD | host combination) |
| CAR Alone | 0 | 100.00 | 0.00 | 14 (each donor of 2 |
| | | | | groups with mismatch |
| | | | | combinations according |
| | | | | to Tables 31B-D) |
| | 2 | 47.74 | 15.16 | 14 |
| | 3 | 73.79 | 28.77 | 14 |
| | 4 | 117.11 | 73.04 | 14 |
| Allo CD70 | 0 | 100.00 | 0.00 | 14 |
| CAR | 2 | 61.63 | 23.08 | 14 |
| | 3 | 140.84 | 78.95 | 14 |
| | 4 | 402.94 | 291.67 | 14 |

TABLE 29
Percent of T cell proliferation with genotypically C matched donors

| | | | | N (donor/ |
|---|---|---|---|---|
| Group | Day | Average | SD | host combination) |
| CAR Alone | 0 | 100.00 | 0.00 | 12 (each donor of 2 |
| | | | | groups with match |
| | | | | combinations according |
| | | | | to Tables 31B-D) |
| | 2 | 37.50 | 8.39 | 12 |
| | 3 | 58.93 | 11.14 | 12 |
| | 4 | 107.07 | 17.74 | 12 |
| Allo CD70 | 0 | 100.00 | 0.00 | 12 |
| CAR | 2 | 55.78 | 9.77 | 12 |
| | 3 | 141.80 | 39.32 | 12 |
| | 4 | 390.11 | 132.37 | 12 |

TABLE 30
Percent of T cell proliferation in
the presence of autologous PBMCs

| | | | | N (donor/ |
|---|---|---|---|---|
| Group | Day | Average | SD | host combination) |
| CAR Alone | 0 | 100.00 | 0.00 | 6 (each donor of 2 |
| | | | | groups with autologous |
| | | | | combinations according |
| | | | | to Tables 31B-D) |
| | 2 | 74.02 | 24.80 | 6 |
| | 3 | 219.25 | 60.51 | 6 |
| | 4 | 1096.42 | 363.05 | 6 |
| Allo CD70 | 0 | 100.00 | 0.00 | 6 |
| CAR | 2 | 85.40 | 28.71 | 6 |
| | 3 | 292.69 | 139.08 | 6 |
| | 4 | 1074.38 | 443.94 | 6 |

Tables 31A-31D. Donor T cell and Host PBMCs Alleles

TABLE 31A
Donor T cells Alleles

| Donor | HLA-A | | HLA-B | | HLA-C | |
|---|---|---|---|---|---|---|
| DonorT1 | A*02:01 | A*23:01 | B*15:01 | B*44:03 | C*04:01 | C*04:01 |
| DonorT2 | A*03:02 | A*03:02 | B*08:01 | B*08:01 | C*07:01 | C*07:01 |
| DonorT3 | A*02:01 | A*03:01 | B*07:02 | B*07:02 | C*07:02 | C*07:02 |

TABLE 31B
Host PBMC's Alleles used with DonorT2

| | | HLA-A | | HLA-B | | HLA-C | |
|---|---|---|---|---|---|---|---|
| Mismatch | HostPBMC1MM | A*24:02 | A*68:01 | B*40:01 | B*51:01 | C*03:04 | C*15:02 |
| | HostPBMC2MM | A*33:01 | A*33:03 | B*15:16 | B*78:01 | C*14:02 | C*16:01 |
| HLA-C | HostPBMC1Matched | A*03:01 | A*11:01 | B*35:01 | B*49:01 | C*04:01 | C*07:01 |
| Match | HostPBMC2Matched | A*02:01 | A*24:02 | B*39:02:02 | B*51:01:01 | C*07:01:01 | C*07:02:01 |
| Autologous | Identical to DonorT2 | A*03:02 | A*03:02 | B*08:01 | B*08:01 | C*07:01 | C*07:01 |

TABLE 31C
Host PBMC's Alleles used with DonorT1

| | | HLA-A | | HLA-B | | HLA-C | |
|---|---|---|---|---|---|---|---|
| Mismatch | HostPBMC1MM | A*24:02 | A*68:01 | B*40:01 | B*51:01 | C*03:04 | C*15:02 |
| | HostPBMC2MM | A*33:01 | A*33:03 | B*15:16 | B*78:01 | C*14:02 | C*16:01 |
| HLA-C | HostPBMC3Matched | A*02:06 | A*68:01 | B*35:01 | B*40:02 | C*03:07 | C*04:01 |
| Match | HostPBMC4Matched | A*24:02 | A*68:35 | B*35:02 | B*35:03 | C*04:01 | C*04:01 |
| Autologous | Identical to DonorT1 | A*02:01 | A*23:01 | B*15:01 | B*44:03 | C*04:01 | C*04:01 |

TABLE 31D
Host PBMC's Alleles used with DonorT3

| | | HLA-A | | HLA-B | | HLA-C | |
|---|---|---|---|---|---|---|---|
| Mismatch | HostPBMC1MM | A*24:02 | A*68:01 | B*40:01 | B*51:01 | C*03:04 | C*15:02 |
| | HostPBMC2MM | A*33:01 | A*33:03 | B*15:16 | B*78:01 | C*14:02 | C*16:01 |
| | HostPBMC3MM | A*11:01 | A*33:01 | B*14:02 | B*40:01 | C*03:04 | C*08:02 |
| HLA-C | HostPBMC2Matched | A*02:01 | A*24:02 | B*39:02:02 | B*51:01:01 | C*07:01:01 | C*07:02:01 |
| Match | HostPBMC5Matched | A*31:01 | A*68:03 | B*39:05 | B*39:05 | C*07:02 | C*07:02 |
| Autologous | Identical to DonorT3 | A*02:01 | A*03:01 | B*07:02 | B*07:02 | C*07:02 | C*07:02 |

TABLE 32
Engineered T cell Donor Groups and Description of Edits

| Donor | Group Name | Edits |
|---|---|---|
| DonorT3 | CAR Alone | CD70/TRAC KO + CD70 CAR |
| DonorT3 | Allo CD70 CAR | TGFBR2/TRAC/CIITA/CD70/ |
| | | HLA-A/HLA-B KO + CD70 CAR |
| DonorT1 | CAR Alone | CD70/TRAC KO + CD70 CAR |
| DonorT1 | Allo CD70 CAR | TGFBR2/TRAC/CIITA/CD70/ |
| | | HLA-A/HLA-B KO + CD70 CAR |
| DonorT2 | CAR Alone | CD70/TRAC KO + CD70 CAR |
| DonorT2 | Allo CD70 CAR | TGFBR2/TRAC/CIITA/CD70/ |
| | | HLA-A/HLA-B KO + CD70 CAR |

1. An anti-CD70 chimeric antigen receptor (CAR) comprising:
a) an antigen-binding protein, or a fragment thereof, that specifically binds to CD70, wherein the antigen-binding protein comprises a heavy chain variable (VH) region comprising a complementarity determining region 1 (VH CDR1), VH CDR2, and VH CDR3, and a light chain variable (VL) region comprising a complementarity determining region 1 (VL CDR1), VL CDR2, and VL CDR3, and wherein
1)
i) the VH CDR1 comprises an amino acid sequence of any one of SEQ ID NOs: 73, 67, 70, 76, 79, and 82;
ii) the VH CDR2 comprises an amino acid sequence of any one of SEQ ID NOs: 74, 68, 71, 77, 80, and 83;
iii) the VH CDR3 comprises an amino acid sequence of any one of SEQ ID NOs: 75, 69, 72, 78, 81, and 84;
iv) the VL CDR1 comprises an amino acid sequence of any one of SEQ ID NOs: 55, 49, 52, 58, 61, and 64;
v) the VL CDR2 comprises an amino acid sequence of any one of SEQ ID NOs: 56, 50, 53, 59, 62, and 65; and
vi) the VL CDR3 comprises an amino acid sequence of any one of SEQ ID NOs: 57, 51, 54, 60, 63, and 66; and/or
2) wherein the VH CDR1, VH CDR2, VH CDR3, VL CDR1, VL CDR2, and VL CDR3 comprise the amino acid sequences of:
i) SEQ ID NOs: 73, 74, 75, 55, 56, and 57, respectively;
ii) SEQ ID NOs: 67, 68, 69, 49, 50, and 51, respectively;
iii) SEQ ID NOs: 70, 71, 72, 52, 53, and 54, respectively;
iv) SEQ ID NOs: 76, 77, 78, 58, 59, and 60, respectively;
y) SEQ ID NOs: 79, 80, 81, 61, 62, and 63, respectively; or
vi) SEQ ID NOs: 82, 83, 84, 64, 65, and 66, respectively;
b) a transmembrane domain; and
c) an intracellular domain comprising a costimulatory domain comprising an amino acid sequence of SEQ ID NO: 99 or 101.

2. (canceled)

3. (canceled)

4. The anti-CD70 CAR of claim 1, wherein the VH region comprises an amino acid sequence having at least 90%, 93%, 95%, 96%, 97%, 98%, or 99% identity to any one of SEQ ID NOs: 43-48 and/or wherein the VL region comprises an amino acid sequence having at least 90%, 93%, 95%, 96%, 97%, 98%, or 99% identity to any one of SEQ ID NOs: 37-42.

5. (canceled)

6. The anti-CD70 CAR of claim 1, wherein:
(a) the VH region comprises an amino acid sequence having at least 90%, 93%, 95%, 96%, 97%, 98%, or 99% identity to SEQ ID NO: 45 or comprises the amino acid sequence of SEQ ID NO: 45, and the VL region comprises an amino acid sequence having at least 90%, 93%, 95%, 96%, 97%, 98%, or 99% identity to SEQ ID NO: 39 or comprises the amino acid sequence of SEQ ID NO: 39;
(b) the VH region comprises an amino acid sequence having at least 90%, 93%, 95%, 96%, 97%, 98%, or 99% identity to SEQ ID NO: 43 or comprises the amino acid sequence of SEQ ID NO: 43, and the VL region comprises an amino acid sequence having at least 90%, 93%, 95%, 96%, 97%, 98%, or 99% identity to SEQ ID NO: 37 or comprises the amino acid sequence of SEQ ID NO: 37;
(c) the VH region comprises an amino acid sequence having at least 90%, 93%, 95%, 96%, 97%, 98%, or 99% identity to SEQ ID NO: 44 or comprises the amino acid sequence of SEQ ID NO: 44, and the VL region comprises an amino acid sequence having at least 90%, 93%, 95%, 96%, 97%, 98%, or 99% identity to SEQ ID NO: 38 or comprises the amino acid sequence of SEQ ID NO: 38;
(d) the VH region comprises an amino acid sequence having at least 90%, 93%, 95%, 96%, 97%, 98%, or 99% identity to SEQ ID NO: 46 or comprises the amino acid sequence of SEQ ID NO: 46, and the VL region comprises an amino acid sequence having at least 90%, 93%, 95%, 96%, 97%, 98%, or 99% identity to SEQ ID NO: 40 or comprises the amino acid sequence of SEQ ID NO: 40;
(e) the VH region comprises an amino acid sequence having at least 90%, 93%, 95%, 96%, 97%, 98%, or 99% identity to SEQ ID NO: 47 or comprises the amino acid sequence of SEQ ID NO: 47, and the VL region comprises an amino acid sequence having at least 90%, 93%, 95%, 96%, 97%, 98%, or 99% identity to SEQ ID NO: 41 or comprises the amino acid sequence of SEQ ID NO: 41; or
(f) the VH region comprises an amino acid sequence having at least 90%, 93%, 95%, 96%, 97%, 98%, or 99% identity to SEQ ID NO: 48 or comprises the amino acid sequence of SEQ ID NO: 48, and the VL region comprises an amino acid sequence having at least 90%, 93%, 95%, 96%, 97%, 98%, or 99% identity to SEQ ID NO: 42 or comprises the amino acid sequence of SEQ ID NO: 42.

7. (canceled)

8. (canceled)

9. (canceled)

10. The anti-CD70 CAR of claim 1, wherein the antigen-binding protein comprises an amino acid sequence of any one of SEQ ID NOs: 25, 27, 29, 32, 34, and 36, or a sequence having at least 90%, 93%, 95%, 96%, 97%, 98%, or 99% identity to any one of SEQ ID NOs: 25, 27, 29, 32, 34, and 36; or
wherein the antigen-binding protein comprises the amino acid sequence of SEQ ID NO: 25 or 27.

11. (canceled)

12. The anti-CD70 CAR of claim 1,
a) wherein the transmembrane domain comprises a CD8a transmembrane domain comprising an amino acid sequence of SEQ ID NO: 95, or wherein the transmembrane domain comprises a CD28 transmembrane domain comprising an amino acid sequence of SEQ ID NO: 93;
b) wherein the anti-CD70 CAR comprises a hinge domain between the antigen-binding protein and the transmembrane domain;
c) wherein the anti-CD70 CAR comprises a hinge domain between the antigen-binding protein and the transmembrane domain, wherein the hinge domain is a CD8a hinge domain, or fragment thereof, comprising an amino acid sequence of SEQ ID NO: 89;
d) wherein the intracellular domain comprises an activation domain;
e) wherein the intracellular domain comprises an activation domain, wherein the activation domain is a CD3z activation domain comprising an amino acid sequence of SEQ ID NO: 103;
f) wherein the antigen binding protein is an scFv;
g) wherein the antigen binding protein is an scFv, and the scFv comprises a linker between the VH and VL regions;
h) wherein the antigen binding protein is an scFv, the scFv comprises a linker between the VH and VL regions, and the scFv comprises a glycine-serine linker between the VH and VL regions; and/or
i) wherein the antigen binding protein is an scFv, the scFv comprises a glycine-serine linker between the VH and VL regions, and the glycine-serine linker comprises the sequence of SEQ ID NO: 940.

13. (canceled)

14. (canceled)

15. The anti-CD70 CAR of claim 1, wherein:
a) the antigen-binding protein comprises a sequence of SEQ ID NO: 32 and the costimulatory domain comprises a sequence of SEQ ID NO: 101;
b) the antigen-binding protein comprises a sequence of SEQ ID NO: 32 and the costimulatory domain comprises a sequence of SEQ ID NO: 99;
c) the antigen-binding protein comprises a sequence of SEQ ID NO: 25 and the costimulatory domain comprises a sequence of SEQ ID NO: 101;
d) the antigen-binding protein comprises a sequence of SEQ ID NO: 25 and the costimulatory domain comprises a sequence of SEQ ID NO: 99;
e) the antigen-binding protein comprises a sequence of SEQ ID NO: 27 and the costimulatory domain comprises a sequence of SEQ ID NO: 101;
f) the antigen-binding protein comprises a sequence of SEQ ID NO: 27 and the costimulatory domain comprises a sequence of SEQ ID NO: 99;
g) the antigen-binding protein comprises a sequence of SEQ ID NO: 36 and the costimulatory domain comprises a sequence of SEQ ID NO: 101; or
h) the antigen-binding protein comprises a sequence of SEQ ID NO: 36 and the costimulatory domain comprises a sequence of SEQ ID NO: 99.

16. The anti-CD70 CAR of claim 1, comprising an amino acid sequence that is at least 95% identical to the amino acid sequence of any one of SEQ ID NOs: 2, 4, 6, 8, 14, 16, 20, and 22.

17. (canceled)

18. (canceled)

19. (canceled)

20. A nucleic acid encoding the anti-CD70 CAR of claim 1.

21. The nucleic acid of claim 20, comprising:
a) a nucleic acid sequence of any one of SEQ ID NOs: 23, 24, 26, 28, 30, 31, 33, and 35, or a nucleic acid sequence that has at least 90%, 93%, 95%, 96%, 97%, 98%, or 99% identity to any one of SEQ ID NOs: 23, 24, 26, 28, 30, 31, 33, and 35;
b) a nucleic acid sequence of any one of SEQ ID NOs: 96-98 and 100, or a nucleic acid sequence that has at least 90%, 93%, 95%, 96%, 97%, 98%, or 99% identity to any one of SEQ ID NOs: 96-98 and 100;
c) a nucleic acid encoding a hinge domain comprised in the anti-CD70 CAR comprising the nucleic acid sequence of any one of SEQ ID NOs: 85-88 or a nucleic acid sequence that has at least 90%, 93%, 95%, 96%, 97%, 98%, or 99% identity to any one of SEQ ID NOs: 85-88;
d) a nucleic acid sequence encoding the transmembrane domain comprising a sequence of any one of SEQ ID NOs: 90-92 and 94 or a sequence having at least 90%, 93%, 95%, 96%, 97%, 98%, or 99% identity to any one of SEQ ID NOS: 90-92 and 94; and/or
e) a nucleic acid encoding an activation domain comprised in the anti-CD70 CAR comprising the nucleic acid sequence of SEQ ID NO: 102 or 104 or a nucleic acid sequence that has at least 90%, 93%, 95%, 96%, 97%, 98%, or 99% identity to SEQ ID NO: 102 or 104.

22. (canceled)

23. (canceled)

24. (canceled)

25. (canceled)

26. The nucleic acid of claim 1, comprising a nucleic acid sequence of any one of SEQ ID NOs: 1, 3, 5, 7, 9, 11, 13, 15, 17, 19, and 21, or a nucleic acid sequence that has at least 90%, 93%, 95%, 96%, 97%, 98%, or 99% identity to any one of SEQ ID NOs: 1, 3, 5, 7, 9, 11, 13, 15, 17, 19, and 21.

27. (canceled)

28. (canceled)

29. An expression vector operably linked to or comprising the nucleic acid of claim 20; and
a) wherein the expression vector comprises a retroviral or lentiviral expression vector;
b) wherein the expression vector comprises an AAV vector;
c) wherein the expression vector comprises a non-viral vector;
d) wherein the expression vector comprises SEQ ID NO: 106; or
e) wherein the expression vector comprises SEQ ID NO: 107.

30. (canceled)

31. (canceled)

32. (canceled)

33. (canceled)

34. (canceled)

35. An engineered cell comprising an anti-CD70 chimeric antigen receptor (CAR), wherein the anti-CD70 CAR comprises:
a) an antigen-binding protein, or a fragment thereof, that specifically binds to CD70, wherein the antigen-binding protein comprises a heavy chain variable (VH) region comprising a complementarity determining region 1 (VH CDR1), VH CDR2, and VH CDR3, and a light chain variable (VL) region comprising a complementarity determining region 1 (VL CDR1), VL CDR2, and VL CDR3, and wherein
1)
i) the VH CDR1 comprises an amino acid sequence of any one of SEQ ID NOs: 73, 67, 70, 76, 79, and 82;
ii) the VH CDR2 comprises an amino acid sequence of any one of SEQ ID NOs: 74, 68, 71, 77, 80, and 83;
iii) the VH CDR3 comprises an amino acid sequence of any one of SEQ ID NOs: 75, 69, 72, 78, 81, and 84;
iv) the VL CDR1 comprises an amino acid sequence of any one of SEQ ID NOs: 55, 49, 52, 58, 61, and 64;
v) the VL CDR2 comprises an amino acid sequence of any one of SEQ ID NOs: 56, 50, 53, 59, 62, and 65; and
vi) the VL CDR3 comprises an amino acid sequence of any one of SEQ ID NOs: 57, 51, 54, 60, 63, and 66; and/or
2) wherein the VH CDR1, VH CDR2, VH CDR3, VL CDR1, VL CDR2, and VL CDR3 comprise the amino acid sequences of:
i) SEQ ID NOs: 73, 74, 75, 55, 56, and 57, respectively;
ii) SEQ ID NOs: 67, 68, 69, 49, 50, and 51, respectively;
iii) SEQ ID NOs: 70, 71, 72, 52, 53, and 54, respectively;
iv) SEQ ID NOs: 76, 77, 78, 58, 59, and 60, respectively;
v) SEQ ID NOs: 79, 80, 81, 61, 62, and 63, respectively; or
vi) SEQ ID NOs: 82, 83, 84, 64, 65, and 66, respectively;
b) a transmembrane domain; and
c) an intracellular domain comprising a costimulatory domain comprising an amino acid sequence of SEQ ID NO: 99 or 101.

36. (canceled)

37. (canceled)

38. (canceled)

39. (canceled)

40. The engineered cell of claim 35, wherein:
(a) the VH region comprises an amino acid sequence having at least 90%, 93%, 95%, 96%, 97%, 98%, or 99% identity to SEQ ID NO: 45 or comprises the amino acid sequence of SEQ ID NO: 45, and the VL region comprises an amino acid sequence having at least 90%, 93%, 95%, 96%, 97%, 98%, or 99% identity to SEQ ID NO: 39 or comprises the amino acid sequence of SEQ ID NO: 39;
(b) the VH region comprises an amino acid sequence having at least 90%, 93%, 95%, 96%, 97%, 98%, or 99% identity to SEQ ID NO: 43 or comprises the amino acid sequence of SEQ ID NO: 43, and the VL region comprises an amino acid sequence having at least 90%, 93%, 95%, 96%, 97%, 98%, or 99% identity to SEQ ID NO: 37 or comprises the amino acid sequence of SEQ ID NO: 37;
(c) the VH region comprises an amino acid sequence having at least 90%, 93%, 95%, 96%, 97%, 98%, or 99% identity to SEQ ID NO: 44 or comprises the amino acid sequence of SEQ ID NO: 44, and the VL region comprises an amino acid sequence having at least 90%, 93%, 95%, 96%, 97%, 98%, or 99% identity to SEQ ID NO: 38;
(d) the VH region comprises an amino acid sequence having at least 90%, 93%, 95%, 96%, 97%, 98%, or 99% identity to SEQ ID NO: 46 or comprises the amino acid sequence of SEQ ID NO: 46, and the VL region comprises an amino acid sequence having at least 90%, 93%, 95%, 96%, 97%, 98%, or 99% identity to SEQ ID NO: 40 or comprises the amino acid sequence of SEQ ID NO: 40;
(e) the VH region comprises an amino acid sequence having at least 90%, 93%, 95%, 96%, 97%, 98%, or 99% identity to SEQ ID NO: 47 or comprises the amino acid sequence of SEQ ID NO: 47, and the VL region comprises an amino acid sequence having at least 90%, 93%, 95%, 96%, 97%, 98%, or 99% identity to SEQ ID NO: 41 or comprises the amino acid sequence of SEQ ID NO: 41; or
(f) the VH region comprises an amino acid sequence having at least 90%, 93%, 95%, 96%, 97%, 98%, or 99% identity to SEQ ID NO: 48 or comprises the amino acid sequence of SEQ ID NO: 48, and the VL region comprises an amino acid sequence having at least 90%, 93%, 95%, 96%, 97%, 98%, or 99% identity to SEQ ID NO: 42 or comprises the amino acid sequence of SEQ ID NO: 42.

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. A population of cells, wherein the population of cells comprises the engineered cell of claim 35.

55. The engineered cell or population of cells of claim 35,
a) wherein the engineered cell further comprises reduced or eliminated surface expression of TGFBR² relative to an unmodified cell;
b) wherein the engineered cell comprises a genetic modification in the TGFBR² gene, and the genetic modification comprises at least one nucleotide within the genomic coordinates chr3: 30606864-30691614;
c) wherein the engineered cell comprises a genetic modification in the TGFBR² gene, and the genetic modification is within the genomic coordinates chr3: 30674205-30674229;
d) wherein the engineered cell comprises a genetic modification in the TGFBR² gene, and the genetic modification comprises at least one nucleotide within the genomic coordinates targeted by a TGFBR² guide RNA comprising a guide sequence of SEQ ID NO: 301;
e) wherein the engineered cell comprises a genetic modification in the TGFBR² gene, and the genetic modification is within the genomic coordinates chr3: 30671941-30671961;
f) wherein the engineered cell comprises a genetic modification in the TGFBR² gene, and the genetic modification comprises at least one nucleotide within the genomic coordinates targeted by a TGFBR² guide RNA comprising a guide sequence of SEQ ID NO: 302;
g) wherein the engineered cell further comprises reduced or eliminated surface expression of CD70 relative to an unmodified cell;
h) wherein the engineered cell comprises a genetic modification in the CD70 gene, and the genetic modification comprises at least one nucleotide within the genomic coordinates chr19: 6586002-6591018;
i) wherein the engineered cell comprises a genetic modification in the CD70 gene, and the genetic modification is within the genomic coordinates: chr19: 6590121-6590145 or chr19: 6586268-6586292;
j) wherein the engineered cell comprises a genetic modification in the CD70 gene, and the genetic modification comprises at least one nucleotide within the genomic coordinates targeted by a CD70 guide RNA comprising a guide sequence of SEQ ID NO: 310 or 311;
k) wherein the engineered cell comprises a genetic modification in the CD70 gene, and the genetic modification comprises at least one nucleotide within the genomic coordinates: chr19: 6590998-6591018 or chr19: 6590991-6591011;
l, wherein the engineered cell comprises a genetic modification in the CD70 gene, and the genetic modification comprises at least one nucleotide within the genomic coordinates targeted by a CD70 guide RNA comprising a guide sequence of SEQ ID NO: 312 or 313;
m) wherein the engineered cell further comprises reduced or eliminated surface expression of HLA-A relative to an unmodified cell;
n) wherein the engineered cell comprises a genetic modification in the HLA-A gene, and the genetic modification comprises at least one nucleotide within the genomic coordinates: (i) chr6: 29942854-29942913 and chr6: 29943518-29943619; and (ii) chr6: 29942540-29945459;
o, wherein the engineered cell comprises a genetic modification in the HLA-A gene, and the genetic modification comprises at least one nucleotide within the genomic coordinates chosen from chr6: 29942891-29942915; and
chr6: 29942609-29942633;
p) wherein the engineered cell comprises a genetic modification in the HLA-A gene, and the genetic modification comprises at least one nucleotide within the genomic coordinates targeted by an HLA-A guide RNA comprising a guide sequence of SEQ ID NO: 403 or 404;
g) wherein the engineered cell further comprises reduced or eliminated surface expression of HLA-B relative to an unmodified cell;
r) wherein the engineered cell comprises a genetic modification in the HLA-B gene, and the genetic modification comprises at least one nucleotide within the genomic coordinates chosen from (i) chr6: 31354480-31357174 and (ii) chr6: 31357084-31354647;
s) wherein the engineered cell comprises a genetic modification in the HLA-B gene, and the genetic modification comprises at least one nucleotide within the genomic coordinates chosen from chr6: 31355222-31355246, chr6: 31355221-31355245, and chr6: 31355205-31355229;
t) wherein the engineered cell comprises a genetic modification in the HLA-B gene, and the genetic modification comprises at least one nucleotide within the genomic coordinates targeted by an HLA-B guide RNA comprising a guide sequence of SEQ ID NO: 406, 405, or 407;
u) wherein the engineered cell further comprises reduced or eliminated surface expression of TRAC relative to an unmodified cell;
v) wherein the engineered cell comprises a genetic modification in the TRAC gene, and the genetic modification comprises at least one nucleotide within the genomic coordinates chr14: 22547524-22547544;
w) wherein the engineered cell comprises a genetic modification in the TRAC gene, and the genetic modification comprises at least one nucleotide within the genomic coordinates targeted by a TRAC guide RNA comprising a guide sequence of SEQ ID NO: 413;
x) wherein the engineered cell further comprises reduced or eliminated surface expression of MHC class II relative to an unmodified cell;
y) wherein the engineered cell comprises a genetic modification in the CIITA gene, and the genetic modification is within the genetic coordinates chosen from: (i) chr16: 10877363-10907788 and (ii) chr16: 10906515-10908136;
z) wherein the engineered cell comprises a genetic modification in the CIITA gene, and the genetic modification is within the genomic coordinates chr16: 10906643-10906667 or chr16: 10907504-10907528;
aa) wherein the engineered cell comprises a genetic modification in the CIITA gene, and the genetic modification comprises at least one nucleotide within the genomic coordinates targeted by a CIITA guide RNA comprising a guide sequence of SEQ ID NO: 402 or 401;
bb) wherein the engineered cell is homozygous for HLA-C; and/or
cc) wherein the engineered cell comprises a genetic modification in the TRAC gene, and the genetic modification comprises an insertion of a heterologous coding sequence.

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. A pharmaceutical composition comprising the engineered cell of claim 35.

80. (canceled)

81. The engineered cell of claim 35, wherein the engineered cell is
(a) an immune cell;
(b) an NK cell;
(c) a T-cell;
(d) a CD4+ T-cell;
(e) a CD8+ T-cell; or
(f) a T-cell that has a T memory stem cell (Tscm) phenotype.

82. (canceled)

83. (canceled)

84. (canceled)

85. (canceled)

86. (canceled)

87. (canceled)

88. (canceled)

89. (canceled)

90. (canceled)

91. (canceled)

92. A method of making an engineered cell comprising
1)
a) contacting a cell with the expression vector of claim 29; and
b) contacting the cell with at least one genomic editing tool comprising a genomic editor and at least one guide RNA, wherein the at least one guide RNA targets a genomic locus chosen from the HLA-A, HLA-B, TRAC, CIITA, TGFBR2, and CD70 locus;
2)
a) providing an engineered cell which has reduced or eliminated surface expression of one or both of TGFBR² and CD70 relative to an unmodified cell; and
b) contacting the cell with the expression vector of claim 29, wherein the engineered cell comprising an anti-CD70 CAR; or
3)
a) providing an engineered cell which has reduced or eliminated surface expression of one or more of HLA-A, HLA-B, MHC Class II, TRAC, TGFBR2, and CD70, relative to an unmodified cell; and
b) contacting the cell with the expression vector of claim 29, wherein the engineered cell comprising an anti-CD70 CAR.

93. (canceled)

94. (canceled)

95. A method of making an engineered cell comprising an anti-CD70 CAR, comprising:
1)
(a) contacting the cell with a first group of lipid nanoparticles (LNPs) comprising a LNP comprising a uracil glycosylase inhibitor (UGI) and at least one LNP comprising a base editor and at least one guide RNA that is cognate to the base editor and targets a genomic locus chosen from the HLA-A, HLA-B, TRAC, CIITA, TGFBR2, and CD70 locus; and
(b) contacting a cell with
i) a second group of LNPs comprising a LNP comprising a UGI and at least one LNP comprising a base editor and at least one guide RNA that is cognate to the base editor and targets a genomic locus chosen from the HLA-A, HLA-B, TRAC, CIITA, TGFBR2, and CD70 locus that are different from the at least one guide RNA contained in the first group of LNPs of the step (a); and
ii) at least one LNP comprising a RNA-guided cleavase and at least one gRNAs that is cognate to the RNA-guided cleavase and targets the TRAC locus; and
iii. iii) a nucleic acid encoding an anti-CD70 CAR for insertion into an editing site (e.g., a double strand break) at the TRAC locus; or
2)
(a) contacting a cell with a first group of lipid nanoparticles (LNPs) comprising a first LNP comprising a base editor and a gRNA that targets the HLA-A locus; a second LNP comprising a base editor and comprising a gRNA that targets the HLA-B locus, a third LNP comprising a base editor and comprising a gRNA that targets the CIITA locus, and a fourth LNP comprising a UGI; and
(b) contacting a cell with (i) a second group of LNPs comprising a fifth LNP comprising a base editor and comprising a gRNA that targets the TGFBR² locus; a sixth LNP comprising a base editor and comprising a gRNA that targets the CD70 locus; (ii) a seventh LNP comprising an RNA-guided DNA cleavase and a gRNA that is cognate to the RNA-guide DNA cleavase and targets the TRAC locus, and an eight lipid LNP comprising a UGI; and (iii) a nucleic acid encoding an anti-CD70 CAR for insertion into an editing site (e.g., a double strand break) at the TRAC locus.

96. (canceled)

97. The method of claim 95, wherein the RNA-guided cleavase comprises an S. pyogenes (Spy) Cas9 cleavase, and the base editor comprises an N. meningitidis (Nme) Cas9 nickase.

98. A method of administering the engineered cell of claim 35 to a subject in need thereof, or to a subject as an adoptive cell transfer (ACT) therapy;
or a method of treating a disease or disorder comprising administering the engineered cell of claim 35 to a subject in need thereof, wherein the disease is a cancer, an infectious disease, or an autoimmune disease.

99. (canceled)

100. The method of claim 98, wherein the engineered cell is allogeneic to the subject.

101. (canceled)

102. (canceled)

103. An engineered cell comprising a genetic modification in the HLA-A gene, a genetic modification in the HLA-B gene, a genetic modification in the TRAC gene, a genetic modification in the CIITA gene, a genetic modification in the TGFBR² gene, and/or a genetic modification in the CD70 gene, wherein the genetic modification in the HLA-A gene is within the genomic coordinates chr6: 29942891-29942915; wherein the genetic modification in the HLA-B gene is within the genomic coordinates chr6: 31355222-31355246, chr6: 31355221-31355245, or chr6: 31355205-31355229; wherein the genetic modification in the TRAC gene is within the genomic coordinates chr14: 22547524-22547544; wherein the genetic modification in the CIITA gene is within the genomic coordinates chr16: 10907504-10907528 or chr16: 10906643-10906667; wherein the genetic modification in the TGFBR² gene is within the genomic coordinates chr3: 30674205-30674229 or chr3: 30671941-30671961; and wherein the genetic modification in the CD70 gene is within the genomic coordinates chr19: 6590121-6590145 or chr19: 6590998-6591018, wherein the engineered cell comprises an anti-CD70 chimeric antigen receptor (CAR) or comprises a nucleic acid encoding an anti-CD70 CAR.

104. (canceled)

105. The engineered cell of claim 103, wherein the engineered cell is an engineered human T cell, wherein
(a) the engineered human T cell comprises a genetic modification in the HLA-A gene and reduced or eliminated surface expression of HLA-A relative to an unmodified cell, a genetic modification in the HLA-B gene and reduced or eliminated surface expression of HLA-B relative to an unmodified cell, a genetic modification in the CIITA gene and reduced or eliminated surface expression of MHC class II relative to an unmodified cell, a genetic modification in the TGFBR² gene and reduced or eliminated surface expression of TGFBR² relative to an unmodified cell, and a genetic modification in the CD70 gene and reduced or eliminated surface expression of CD70 relative to an unmodified cell, and (b) the anti-CD70 CAR comprises an antigen-binding protein, or a fragment thereof, that binds to CD70, wherein the antigen-binding protein comprises a heavy chain variable (VH) region comprising a complementarity determining region 1 (VH CDR1) comprising the amino acid sequence of SEQ ID NO: 73, a VH CDR2 comprising the amino acid sequence of SEQ ID NO: 74, and a VH CDR3 comprising the amino acid sequence of SEQ ID NO: 75, and comprises a light chain variable (VL) region comprising a complementarity determining region 1 (VL CDR1) comprising the amino acid sequence of SEQ ID NO: 55, a VL CDR2 comprising the amino acid sequence of SEQ ID NO: 56, and a VL CDR3 comprising the amino acid sequence of SEQ ID NO: 57, wherein the anti-CD70 CAR is inserted into the TRAC locus within the genomic coordinates chr14: 22547524-22547544.

106. (canceled)

107. (canceled)

108. (canceled)

109. (canceled)

110. (canceled)

111. (canceled)

112. A pharmaceutical composition comprising a population of cells, wherein the population comprises the engineered cell of claim 105.

113. (canceled)

114. (canceled)

115. (canceled)

116. (canceled)

117. (canceled)

118. (canceled)

119. (canceled)

120. (canceled)

121. (canceled)

122. The method of claim 98, wherein the cancer is
(a) a solid tumor;
(b) a renal cell carcinoma;
(c) a hematological malignancy;
(d) an acute myeloid leukemia; or
(e) a multiple myeloma.

123. (canceled)
